(19) United States
(12) Patent Application Publication
(10) Pub. No.: US 2025/0171420 A1
(43) Pub. Date: May 29, 2025

(54) MOLECULAR GLUE DEGRADERS AND METHODS OF USING THE SAME

(71) Applicant: Monte Rosa Therapeutics, Inc., Boston, MA (US)

(72) Inventors: Laura Ann McAllister, Basel (CH); Lars Wiedmer, Basel (CH); Ètienne Donckele, Basel (CH); Bernhard Fasching, Basel (CH); Nina Ilic-Widlund, Basel (CH)

(21) Appl. No.: 18/982,527

(22) Filed: Dec. 16, 2024

Related U.S. Application Data
(63) Continuation of application No. PCT/US2023/025589, filed on Jun. 16, 2023.
(60) Provisional application No. 63/352,855, filed on Jun. 16, 2022.

(51) Int. Cl. C07D 401/14; A61K 31/454; A61K 31/4545; A61K 31/4709; A61K 31/4725; A61K 31/496; A61K 31/497; A61K 31/498; A61K 31/506; A61P 35/00; C07D 405/14; C07D 413/14; C07D 417/14; C07D 471/04; C07D 471/10; C07D 487/04; C07D 487/08; C07D 487/10; C07D 491/107

(52) U.S. Cl. CPC C07D 401/14; A61K 31/454; A61K 31/4545; A61K 31/4709; A61K 31/4725; A61K 31/496; A61K 31/497; A61K 31/498; A61K 31/506; A61P 35/00; C07D 405/14; C07D 413/14; C07D 417/14; C07D 471/04; C07D 471/10; C07D 487/04; C07D 487/08; C07D 487/10; C07D 491/107

(57) ABSTRACT

Described herein, in part, are compounds that mediate the degradation of casein kinase 1α (CK1α), and are therefore useful in the treatment of various disorders, such as cancer.

### RELATED APPLICATIONS

This application is a continuation of International Patent Application No. PCT/US2023/025589, filed Jun. 16, 2023, which claims priority to U.S. Provisional Patent Application No. 63/352,855, filed on Jun. 16, 2022, the contents of which are incorporated by reference in their entireties.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as modulators of cereblon to mediate the degradation of a protein, and are therefore are useful in the treatment of disorders, such as cancer. For example, it has been found that compounds of the present disclosure mediate the targeted degradation of the protein casein kinase 1α (CK1α).

In one aspect, described herein is a compound of Formula (I):

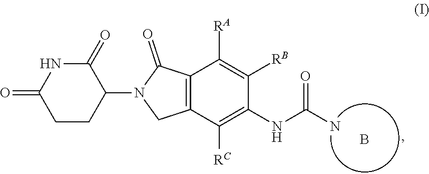

or a pharmaceutically acceptable salt thereof, wherein each of R^{A}, R^{B}, R^{C} is independently selected from the group consisting of H and halogen; and ring B is selected from the group consisting of 4-6 membered monocyclic heterocyclyl, 8-10 membered bicyclic heterocyclyl, 6-11 membered bridged bicyclic heterocyclyl, and 7-14 membered spirocyclic heterocyclyl; wherein 4-6 membered monocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, pyridinyl, pyrimidyl and thiazolyl, wherein the C₁₋₆alkyl, C₁₋₆alkoxy, phenyl, pyrazolyl, pyridinyl, and pyrimidyl may optionally be substituted by one, two or three substituents each independently selected from the group consisting of halogen, hydroxyl, cyano, C₁₋₆alkyl, and C₁₋₆haloalkyl; and each of 8-10 membered bicyclic heterocyclyl, 6-11 membered bridged bicyclic heterocyclyl, and 7-14 membered spirocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, and pyrazolyl, wherein the C₁₋₆alkyl, C₁₋₆haloalkyl, C₃₋₆cycloalkyl, C₁₋₆alkoxy, phenyl, and pyrazolyl is optionally substituted by one, two or three substituents each independently selected from the group consisting of halogen, hydroxyl, cyano, C₁₋₆alkyl, C₁₋₆alkoxy, and C₁₋₆haloalkyl; provided that the compound is not:

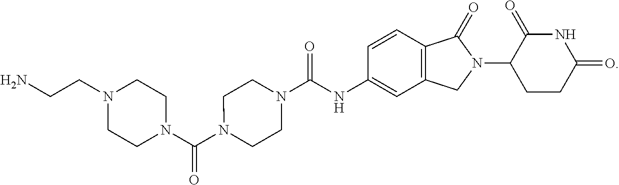 [SMILES: NCCN1CCN(C(=O)N2CCN(C(=O)Nc3ccc4c(c3)CN(C3CCC(=O)NC3=O)C4=O)CC2)CC1]

### BRIEF DESCRIPTION OF THE DRAWINGS

FIG. 1 depicts effects of Compound 102 on CK1a levels in an HEK293 cell line in comparison to effects of Compound 102 on CK1a levels in a cereblon (CRBN) knock-out HEK293 cell line. The data show that Compound 102 decreases CK1a levels in the HEK293 cell line while in the presence of cereblon, whereas Compound 102 demonstrates no effect on CK1a levels in the absence of cereblon in the cereblon knock-out HEK293 cell line.

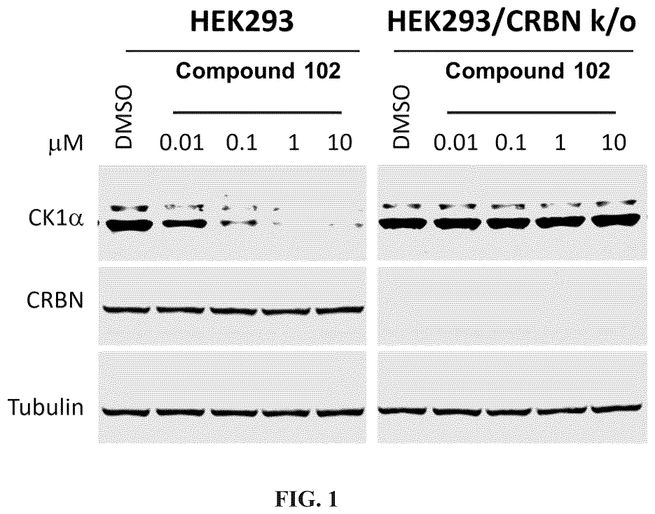

FIG. 2 depicts time-dependent plasma concentrations for Mouse oral PK at 10 mg/kg for compound 288, compound 335, compound 102, and compound 303.

FIG. 3 depicts time-dependent plasma concentrations for Rat oral PK for Compound 288 at 1, 10, 30 mg/kg.

FIG. 4 depicts time-dependent plasma concentrations for oral PK for Compound 288 at 1 mg/kg, for male and female cynomolgus monkeys.

FIG. 5 depicts pharmacodynamics evaluations on protein levels in CK1a and β-actin assays for cynomolgus monkeys at 1 mg/kg Compound 288.

FIG. 6 depicts multi-dose plasma concentrations with time for Compound 288.

FIG. 7 depicts multi-dose xenograft tumor concentrations with time for compound 288 at 1 mg/kg, 10 mg/kg, and 25 mg/kg.

FIG. 8 depicts multi-dose pharmacodynamic data for CK1a and β-actin levels in tumors for compound 288 at 1 mg/kg, 10 mg/kg, and 25 mg/kg.

FIG. 9 depicts multi-dose pharmacodynamic data for p53 and β-actin levels in tumors for compound 288 at 1 mg/kg, 10 mg/kg, and 25 mg/kg.

FIG. 10 depicts multi-dose pharmacodynamic data for p21 and β-actin levels in tumors for compound 288 at 1 mg/kg, 10 mg/kg, and 25 mg/kg.

FIG. 11 depicts multi-dose plasma concentrations with time for Compound 102.

FIG. 12 depicts multi-dose xenograft tumor concentrations with time for compound 102.

FIG. 13 depicts multi-dose pharmacodynamic data for CK1a and GAPDH levels in tumors for Compound 102.

FIG. 14 depicts multi-dose pharmacodynamic data for p53 and GAPDH levels in tumors for Compound 102.

FIG. 15 depicts multi-dose pharmacodynamic data for p21 and GAPDH levels in tumors for Compound 102.

FIG. 16 depicts tumor reduction efficacy at 10 mg/kg in the CW2 cell line for Compound 288.

FIG. 17 depicts tumor reduction efficacy at 10 mg/kg in the HT29 cell line for Compound 288.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

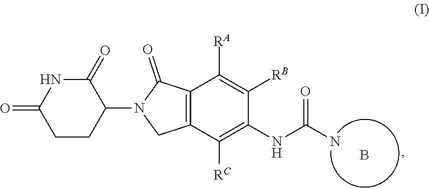

or a pharmaceutically acceptable salt thereof, wherein each of R^{A}, R^{B}, R^{C} is independently selected from the group consisting of H and halogen; and ring B is selected from the group consisting of 4-6 membered monocyclic heterocyclyl, 8-10 membered bicyclic heterocyclyl, 6-11 membered bridged bicyclic heterocyclyl, and 7-14 membered spirocyclic heterocyclyl; wherein 4-6 membered monocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, pyridinyl, pyrimidyl and thiazolyl, wherein the C₁₋₆alkyl, C₁₋₆alkoxy, phenyl, pyrazolyl, pyridinyl, and pyrimidyl may optionally be substituted by one, two or three substituents each independently selected from the group consisting of halogen, hydroxyl, cyano, C₁₋₆alkyl, and C₁₋₆haloalkyl; and each of 8-10 membered bicyclic heterocyclyl, 6-11 membered bridged bicyclic heterocyclyl, and 7-14 membered spirocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, and pyrazolyl, wherein the C₁₋₆alkyl, C₁₋₆haloalkyl, C₃₋₆cycloalkyl, C₁₋₆alkoxy, phenyl, and pyrazolyl is optionally substituted by one, two or three substituents each independently selected from the group consisting of halogen, hydroxyl, cyano, C₁₋₆alkyl, C₁₋₆alkoxy, and C₁₋₆haloalkyl; provided that the compound is not:

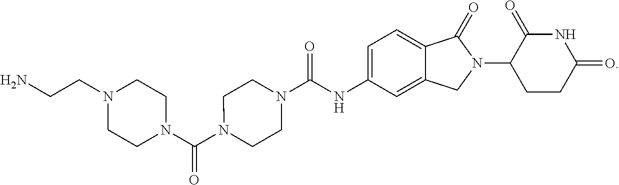 [SMILES: NCCN1CCN(C(=O)N2CCN(C(=O)Nc3ccc4c(c3)CN(C3CCC(=O)NC3=O)C4=O)CC2)CC1]

In one aspect, described herein is a compound of Formula (I):

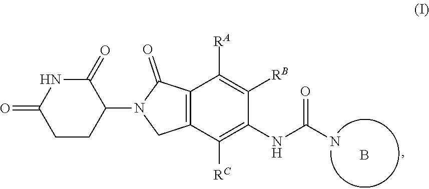

or a pharmaceutically acceptable salt thereof, wherein each of R^{A}, R^{B}, R^{C} is independently selected from the group consisting of H and halogen; and ring B is selected from the group consisting of 4-6 membered monocyclic heterocyclyl, 4-6 membered bridged monocyclic heterocyclyl, 8-10 membered bicyclic heterocyclyl, 6-11 membered bridged bicyclic heterocyclyl, and 7-14 membered spirocyclic heterocyclyl; wherein 4-6 membered monocyclic heterocyclyl or 4-6 membered bridged monocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, bicyclo[1.1.1]pentanyl, phenyl, pyrazolyl, pyridinyl, pyrimidyl and thiazolyl, wherein the C₁₋₆alkyl, C₁₋₆alkoxy, bicyclo[1.1.1]pentanyl, phenyl, pyrazolyl, pyridinyl, and pyrimidyl may optionally be substituted by one, two or three substituents each independently selected from the group consisting of halogen, hydroxyl, cyano, C₁₋₆alkyl, and C₁₋₆haloalkyl;
each of 8-10 membered bicyclic heterocyclyl, 6-11 membered bridged bicyclic heterocyclyl, and 7-14 membered spirocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, piperazinyl, —C(O)N(R^{a}R^{b}), oxetanyl, tetrahydrofuranyl, and tetrahydropyranyl, wherein the C₁₋₆alkyl, C₁₋₆haloalkyl, C₃₋₆cycloalkyl, C₁₋₆alkoxy, phenyl, pyrazolyl, piperazinyl, oxetanyl, tetrahydrofuranyl, and tetrahydropyranyl, is optionally substituted by one, two or three substituents each independently selected from the group consisting of halogen, hydroxyl, cyano, C₁₋₆alkyl, C₁₋₆alkoxy, C₁₋₆haloalkyl, C₁₋₆haloalkoxy, N(R^{a}R^{b}), and 5-6 membered heterocyclyl; and each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure; provided that the compound is not:

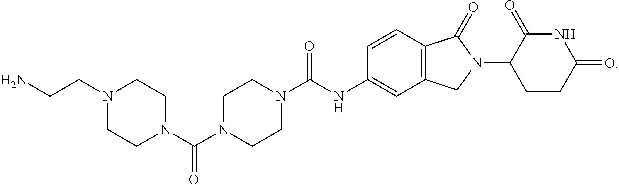 [SMILES: NCCN1CCN(C(=O)N2CCN(C(=O)Nc3ccc4c(c3)CN(C3CCC(=O)NC3=O)C4=O)CC2)CC1]

In some embodiments, each of R^{A}, R^{B}, R^{C} is H. In some embodiments, R^{A} is H. In some embodiments, R^{B} is H. In some embodiments, R^{C} is H.

In some embodiments, each R^{A} and R^{B} is H, and R^{C} is F. In some embodiments, R^{A} is fluoro. In some embodiments, R^{B} is fluoro. In some embodiments, R^{C} is fluoro.

In some embodiments, ring B is 4-6 membered monocyclic heterocyclyl or 4-6 membered bridged monocyclic heterocyclyl. In some embodiments, ring B is 4-6 membered bridged monocyclic heterocyclyl. In some embodiments, ring B is 4-6 membered monocyclic heterocyclyl.

In some embodiments, wherein ring B is 4-6 membered monocyclic heterocyclyl or 4-6 membered bridged monocyclic heterocyclyl optionally substituted by one, two or three substituents each independently selected from the group consisting of hydroxyl, C₁₋₆alkyl, C₁₋₆alkyl-OH, C₁₋₆haloalkyl, C₃₋₆cycloalkyl, bicyclo[1.1.1]pentanyl, phenyl, pyrazolyl, pyridinyl, pyrimidyl and thiazolyl; wherein each occurrence of phenyl, pyrazolyl, pyridinyl, pyrimidyl, and thiazolyl is optionally substituted by one, two or three substituents each independently selected from the group consisting of halogen, hydroxyl, cyano, C₁₋₆alkyl, and C₁₋₆haloalkyl.

In some embodiments, ring B is 4-6 membered monocyclic heterocyclyl optionally substituted by one, two or three substituents each independently selected from the group consisting of hydroxyl, C₁₋₆alkyl, C₁₋₆alkyl-OH, C₁₋₆haloalkyl, C₃₋₆cycloalkyl, bicyclo[1.1.1]pentanyl, phenyl, pyrazolyl, pyridinyl, pyrimidyl, and thiazolyl; wherein each occurrence of phenyl, pyrazolyl, pyridinyl, and pyrimidyl is optionally substituted by one, two or three substituents each independently selected from the group consisting of halogen, hydroxyl, cyano, C₁₋₆alkyl, and C₁₋₆haloalkyl.

In some embodiments, wherein ring B is 4-6 membered bridged monocyclic heterocyclyl optionally substituted by one, two or three substituents each independently selected from the group consisting of hydroxyl, C₁₋₆alkyl, C₁₋₆alkyl-OH, C₁₋₆haloalkyl, C₃₋₆cycloalkyl, bicyclo[1.1.1]pentanyl, phenyl, pyrazolyl, pyridinyl, pyrimidyl and thiazolyl; wherein each occurrence of phenyl, pyrazolyl, pyridinyl, pyrimidyl, and thiazolyl is optionally substituted by one, two or three substituents each independently selected from the group consisting of halogen, hydroxyl, cyano, C₁₋₆alkyl, and C₁₋₆haloalkyl.

In some embodiments, ring B is selected from the group consisting of azetidinyl, pyrrolidinyl, piperidinyl, piperazinyl, and azabicyclo[2.2.1]heptane, wherein ring B is optionally substituted by one, two or three substituents each independently selected from the group consisting of hydroxyl, C₁₋₆alkyl, C₁₋₆alkyl-OH, C₁₋₆haloalkyl, C₃₋₆cycloalkyl, phenyl, pyrazolyl, pyridinyl, pyrimidyl and thiazolyl; wherein each occurrence of C₁₋₆alkyl, C₁₋₆alkyl-OH, C₁₋₆haloalkyl, C₃₋₆cycloalkyl, phenyl, pyrazolyl, pyridinyl, and pyrimidyl is optionally substituted by one, two or three substituents each independently selected from the group consisting of halogen, hydroxyl, cyano, C₁₋₆alkyl, and C₁₋₆haloalkyl.

In some embodiments, ring B is selected from the group consisting of:

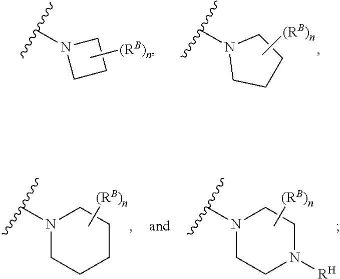

wherein: R^{B}, for each occurrence, is independently selected from the group consisting of hydroxyl, C₁₋₆alkyl, C₁₋₆alkyl-OH, C₁₋₆haloalkyl, C₃₋₆cycloalkyl, phenyl, pyrazolyl, pyridinyl, pyrimidyl and thiazolyl; R^{H} is selected from the group consisting of C₃₋₆cycloalkyl, phenyl, pyrazolyl, pyridinyl, pyrimidyl and thiazolyl, wherein each occurrence of phenyl, pyrazolyl, pyridinyl, and pyrimidyl is optionally substituted by one, two or three substituents each independently selected from the group consisting of halogen, hydroxyl, cyano, C₁₋₆alkyl, and C₁₋₆haloalkyl; and n is 1, 2 or 3.

In some embodiments, ring B is selected from the group consisting of:

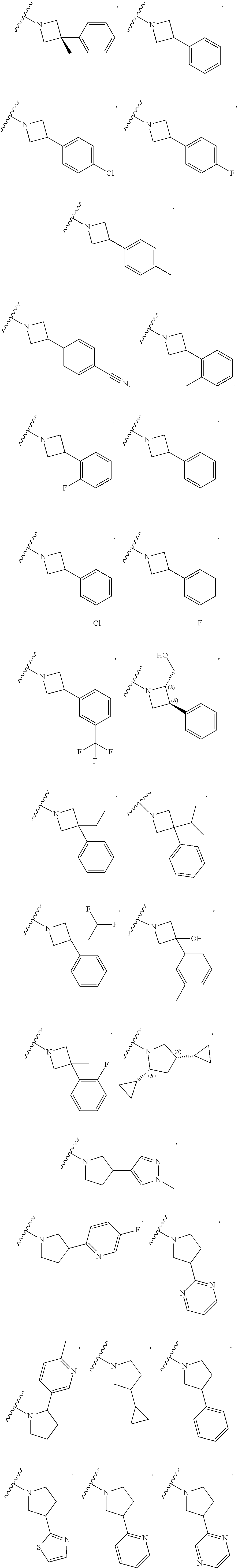

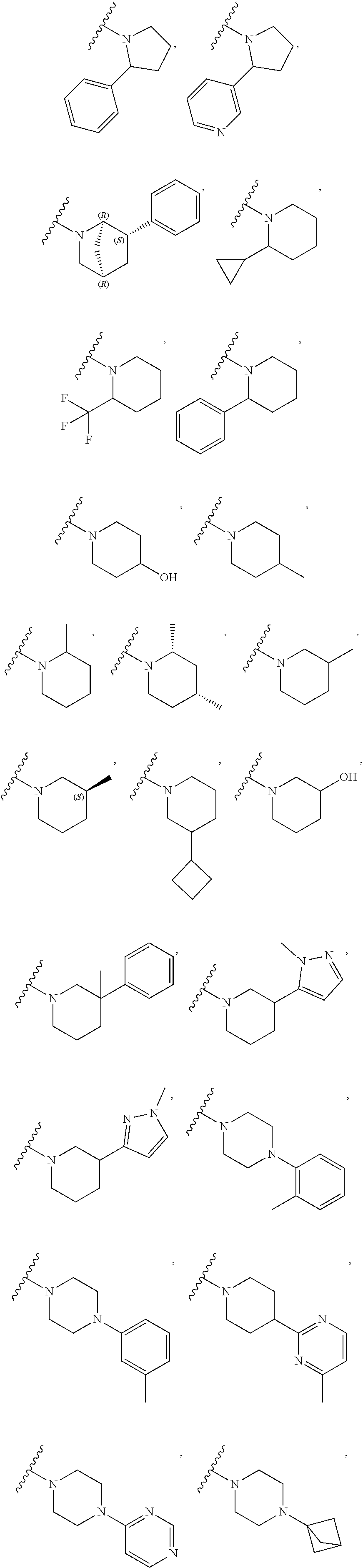

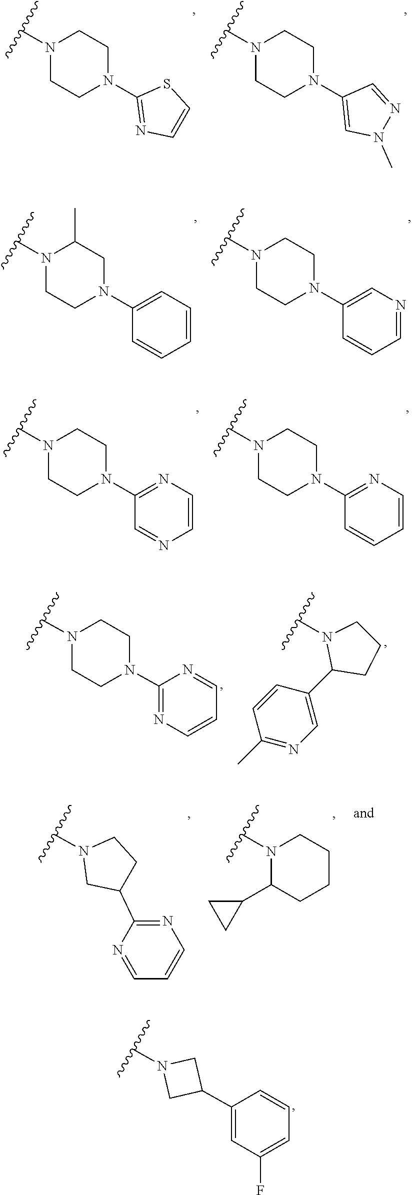

In some embodiments, ring B is 8-10 membered bicyclic heterocyclyl.

In some embodiments, ring B is 8-10 membered bicyclic heterocyclyl optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, piperazinyl, —C(O)N(R^{a}R^{b}), oxetanyl, tetrahydrofuranyl, and tetrahydropyranyl, wherein each occurrence of C₁₋₆alkyl, C₁₋₆alkoxy, phenyl, pyrazolyl, pyrazolyl, piperazinyl, —C(O)N(R^{a}R^{b}), oxetanyl, tetrahydrofuranyl, and tetrahydropyranyl is optionally substituted by one, two or three substituents each independently selected from the group consisting of halogen, hydroxyl, cyano, C₁₋₆alkyl, C₁₋₆haloalkyl, C₁₋₆haloalkoxy, N(R^{a}R^{b}), and 5-6 membered heterocyclyl; and each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure.

In some embodiments, ring B is 8-10 membered bicyclic heterocyclyl optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl and pyrazolyl, wherein each occurrence of C₁₋₆alkyl, C₁₋₆alkoxy, phenyl and pyrazolyl is optionally substituted by one, two or three substituents each independently selected from the group consisting of halogen, hydroxyl, cyano, C₁₋₆alkyl, and C₁₋₆haloalkyl.

In some embodiments, ring B is selected from the group consisting of indolinyl, pyrrolopyridinyl, tetrahydroquinolinyl, and tetrahydronaphthyridine, wherein each occurrence of indolinyl, pyrrolopyridinyl, tetrahydroquinolinyl, and tetrahydronaphthyridine is optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl and pyrazolyl, wherein each occurrence of C₁₋₆alkyl, C₁₋₆alkoxy, phenyl and pyrazolyl is optionally substituted by one, two or three substituents each independently selected from the group consisting of halogen, hydroxyl, cyano, C₁₋₆alkyl, C₁₋₆alkoxy, and C₁₋₆haloalkyl.

In some embodiments, ring B is selected from the group consisting of indolinyl, pyrrolopyridinyl, tetrahydroquinolinyl, and tetrahydronaphthyridine, wherein each occurrence of indolinyl, pyrrolopyridinyl, tetrahydroquinolinyl, and tetrahydronaphthyridine is optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl pyrazolyl, piperazinyl, —C(O)N(R^{a}R^{b}), oxetanyl, tetrahydrofuranyl, and tetrahydropyranyl, wherein each occurrence of C₁₋₆alkyl, C₁₋₆alkoxy, phenyl, pyrazolyl, pyrazolyl, piperazinyl, —C(O)N(R^{a}R^{b}), oxetanyl, tetrahydrofuranyl, and tetrahydropyranyl is optionally substituted by one, two or three substituents each independently selected from the group consisting of halogen, hydroxyl, cyano, C₁₋₆alkyl, C₁₋₆haloalkyl, C₁₋₆haloalkoxy, N(R^{a}R^{b}), and 5-6 membered heterocyclyl; and each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure.

In some embodiments, ring B is selected from the group consisting of:

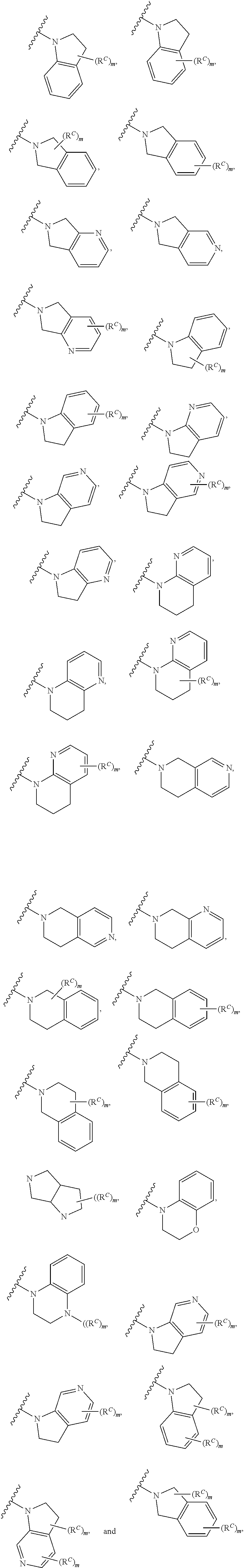

wherein R^{C}, for each occurrence, is independently selected from the group consisting of halogen, cyano, C₁₋₆alkyl, C₁₋₆alkyl-OH, C₁₋₆haloalkyl, C₁₋₆alkoxy, C₃₋₆cycloalkyl, phenyl, pyrazolyl, pyridinyl, pyrimidyl and thiazolyl; wherein each occurrence of C₁₋₆alkoxy, phenyl, pyrazolyl, pyridinyl, and pyrimidyl is optionally substituted by one, two or three substituents each independently selected from the group consisting of halogen, hydroxyl, cyano, C₁₋₆alkyl, C₁₋₆alkoxy, and C₁₋₆haloalkyl; and m is 0, 1, 2 or 3.

In some embodiments, ring B is selected from the group consisting of:

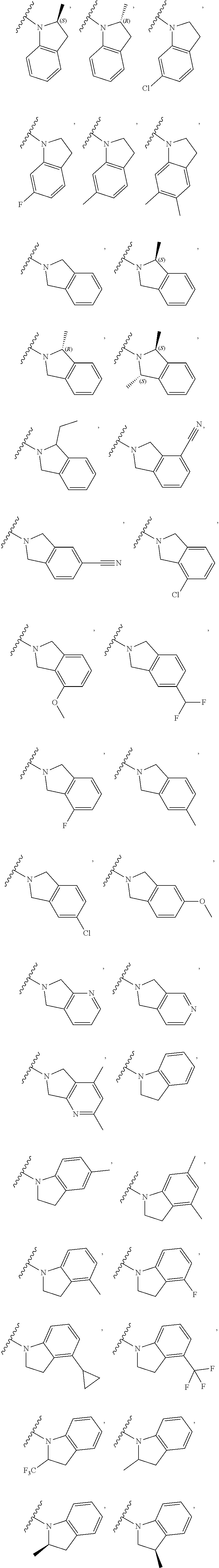

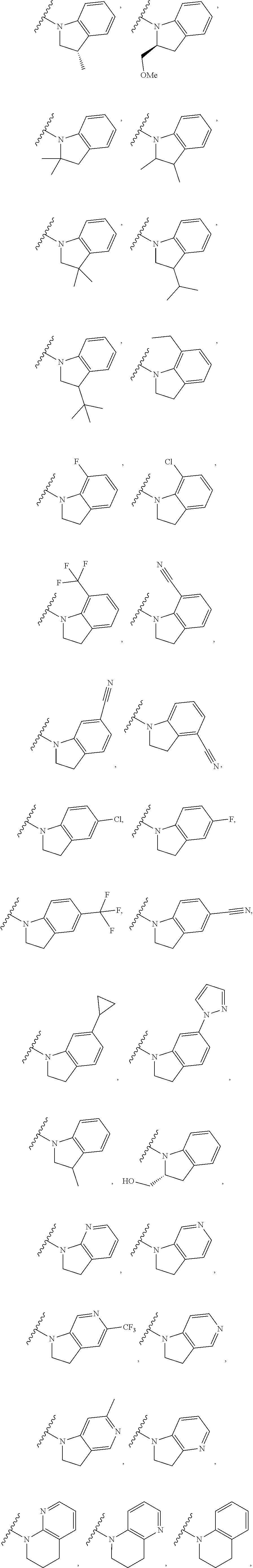

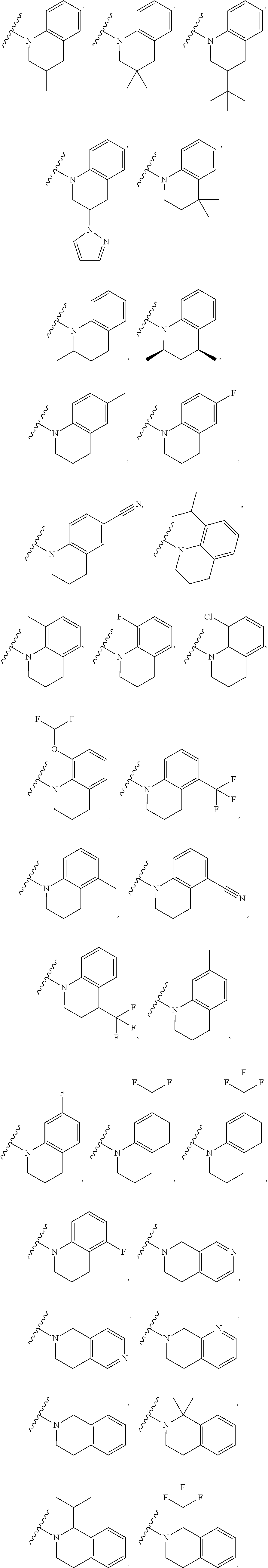

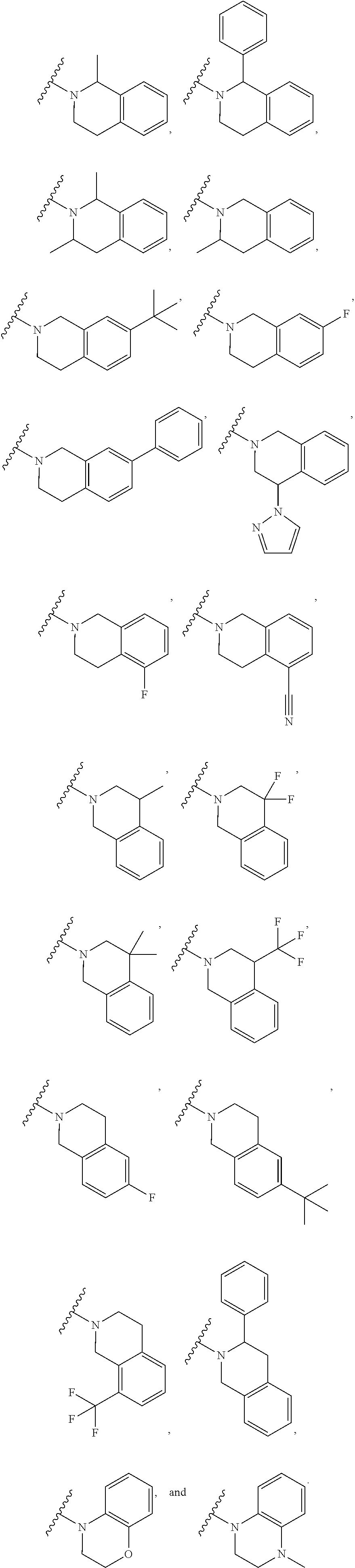

In some embodiments, ring B is 6-11 membered bridged bicyclic heterocyclyl.

In some embodiments, ring B is optionally substituted by one, two or three substituents each independently selected from the group consisting of halogen, C₁₋₆alkyl, C₁₋₆haloalkyl, and phenyl.

In some embodiments, ring B is selected from the group consisting of tetrahydro-epiminonaphthalene, dihydro-epiminonaphthalene, tetrahydro-cyclopropaquinolinyl, azabicycloheptanyl, octahydropyrrolopyrrolyl, azabicyclohexanyl, azabicyclohexanyl, and azabicycloheptanyl, wherein each occurrence of tetrahydro-epiminonaphthalene, dihydro-epiminonaphthalene, tetrahydro-cyclopropaquinolinyl, azabicycloheptanyl, octahydropyrrolopyrrolyl, azabicyclohexanyl, azabicyclohexanyl, and azabicycloheptanyl is optionally substituted by one, two or three substituents each independently selected from the group consisting of halogen, C₁₋₆alkyl, C₁₋₆haloalkyl, and phenyl.

In some embodiments, ring B is selected from the group consisting of:

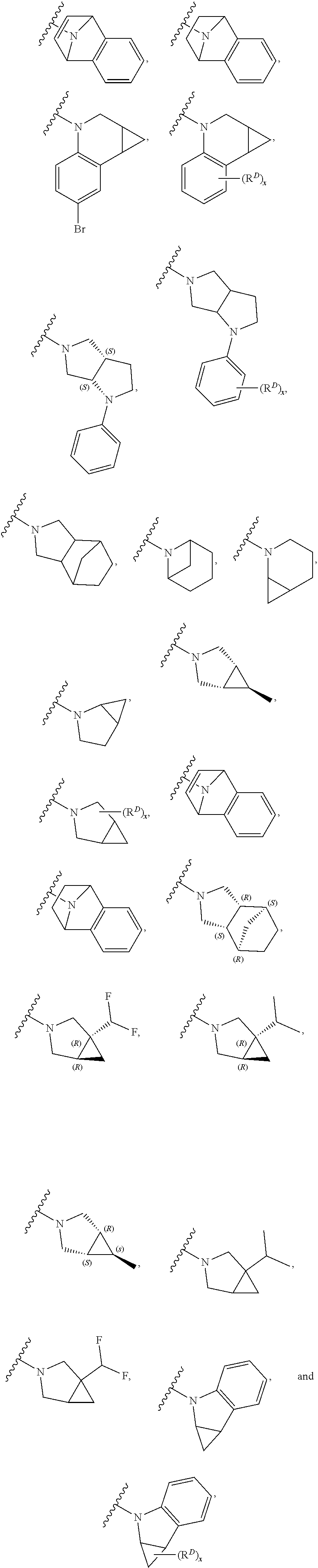

wherein R^{D}, for each occurrence, is independently selected from the group consisting of halogen, C₁₋₆alkyl, C₁₋₆haloalkyl, and phenyl; and
x is 0, 1, or 2.

In some embodiments, ring B is 7-14 membered spirocyclic heterocyclyl.

In some embodiments, ring B is substituted by one, two or three occurrences of C₁₋₆alkyl, C₁₋₆haloalkyl, or C₃₋₆cycloalkyl. In some embodiments, ring B is substituted by one, two or three occurrences of C₁₋₆alkyl.

In some embodiments, ring B is selected from the group consisting of spiro[cyclopropane-1,3′-indolinyl], spiro[cyclobutane-1,3′-indolinyl], spiro[cyclopentane-1,3′-indolinyl], 2′,3′,5′,6′-tetrahydrospiro[indoline-3,4′-pyranyl], 2′,3′-dihydro-1′H-spiro[cyclopropane-1,4′-isoquinolinyl], 6-azaspiro[3.5]nonanyl, 5-azaspiro[2.4]heptanyl, 6-azaspiro[3.4]octanyl, and 2-azabicyclo[4.1.0]heptanyl.

In some embodiments, ring B is selected from the group consisting of:

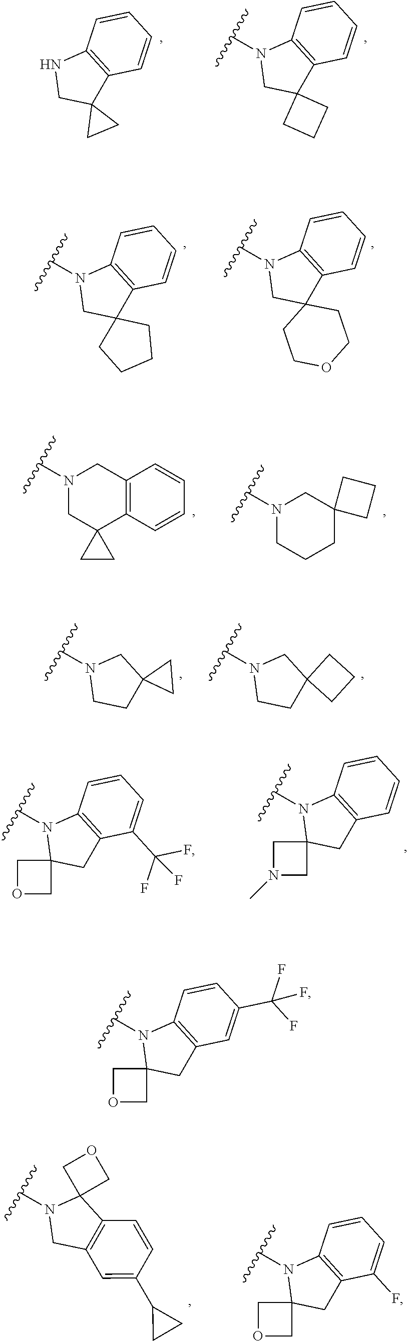

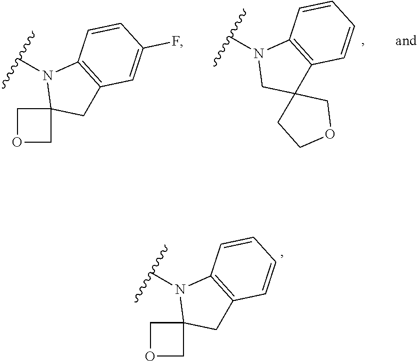

wherein R^{D}, for each occurrence, is independently selected from the group consisting of halogen, C₁₋₆alkyl, C₁₋₆haloalkyl, and phenyl; and x is 0, 1, or 2.

Provided herein, in an aspect, is a compound described in Table 1 below. Table 1 also includes the compound number of each compound in accordance with the contents of the present specification.

In some embodiments, the compound is a compound identified in Table 1 below or a pharmaceutically acceptable salt thereof.

TABLE 1
Exemplary compounds

| Cpmd No. | Structure |
|---|---|
| 101 | 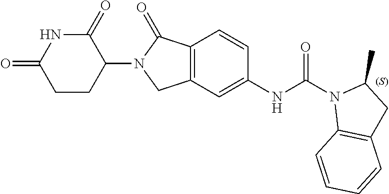 |
| 102 | 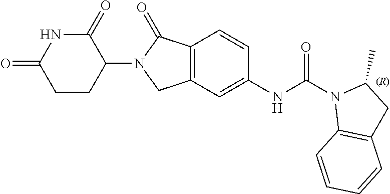 |
| 103 | 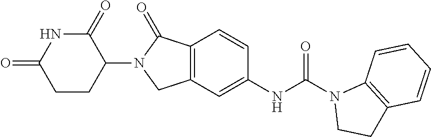 [SMILES: O=C1CCC(N2Cc3cc(NC(=O)N4CCc5ccccc54)ccc3C2=O)C(=O)N1] |
| 104 | 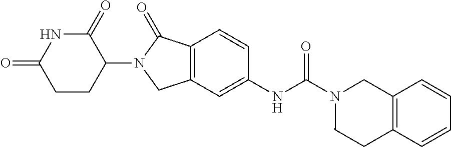 [SMILES: O=C1CCC(N2Cc3cc(NC(=O)N4CCc5ccccc5C4)ccc3C2=O)C(=O)N1] |
| 105 | 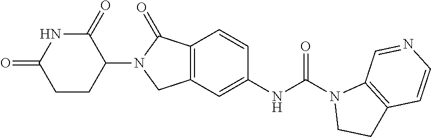 [SMILES: O=C1CCC(N2Cc3cc(NC(=O)N4CCc5ccncc54)ccc3C2=O)C(=O)N1] |
| 106 | 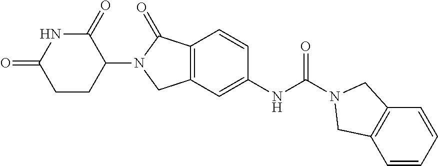 [SMILES: O=C1CCC(N2Cc3cc(NC(=O)N4Cc5ccccc5C4)ccc3C2=O)C(=O)N1] |
| 107 | 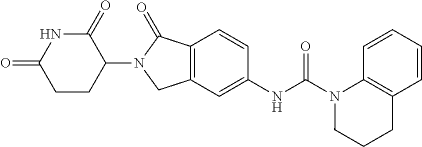 [SMILES: O=C1CCC(N2Cc3cc(NC(=O)N4CCCc5ccccc54)ccc3C2=O)C(=O)N1] |
| 108 | 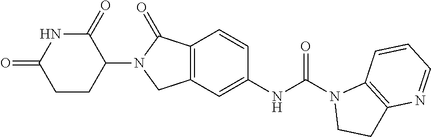 [SMILES: O=C1CCC(N2Cc3cc(NC(=O)N4CCc5ncccc54)ccc3C2=O)C(=O)N1] |

| | |
|---|---|
| 109 | 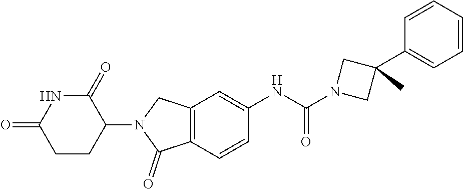 |
| 110 | 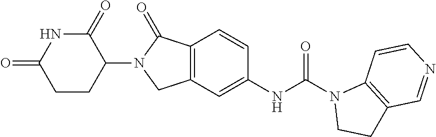 [SMILES: O=C1CCC(N2Cc3cc(NC(=O)N4CCc5cnccc54)ccc3C2=O)C(=O)N1] |
| 111 | 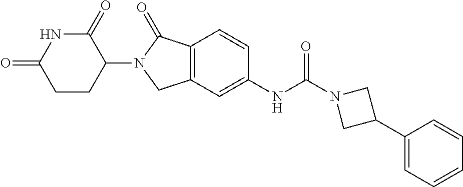 [SMILES: O=C1CCC(N2Cc3cc(NC(=O)N4CC(c5ccccc5)C4)ccc3C2=O)C(=O)N1] |
| 112 | 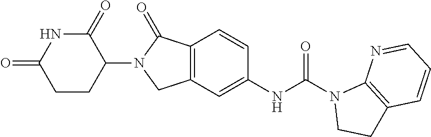 [SMILES: O=C1CCC(N2Cc3cc(NC(=O)N4CCc5cccnc54)ccc3C2=O)C(=O)N1] |
| 113 | 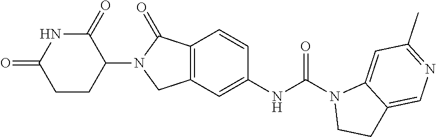 [SMILES: Cc1cc2c(cn1)CCN2C(=O)Nc1ccc2c(c1)CN(C1CCC(=O)NC1=O)C2=O] |
| 114 | 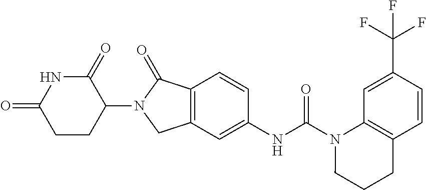 [SMILES: O=C1CCC(N2Cc3cc(NC(=O)N4CCCc5ccc(C(F)(F)F)cc54)ccc3C2=O)C(=O)N1] |
| 115 | 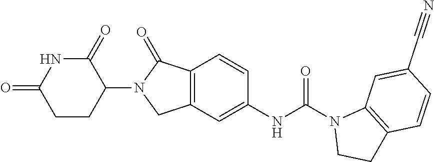 [SMILES: N#Cc1ccc2c(c1)N(C(=O)Nc1ccc3c(c1)CN(C1CCC(=O)NC1=O)C3=O)CC2] |
| 116 | 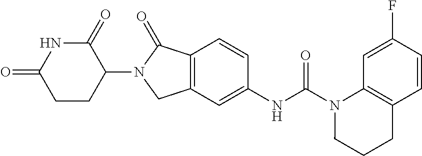 [SMILES: O=C1CCC(N2Cc3cc(NC(=O)N4CCCc5ccc(F)cc54)ccc3C2=O)C(=O)N1] |
| 117 | 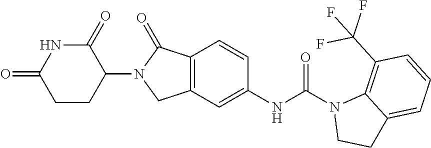 [SMILES: O=C1CCC(N2Cc3cc(NC(=O)N4CCc5cccc(C(F)(F)F)c54)ccc3C2=O)C(=O)N1] |
| 118 | 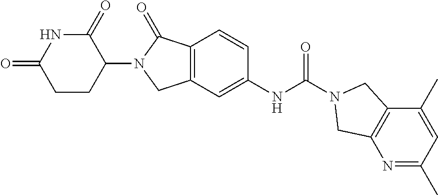 [SMILES: Cc1cc(C)c2c(n1)CN(C(=O)Nc1ccc3c(c1)CN(C1CCC(=O)NC1=O)C3=O)C2] |
| 119 | 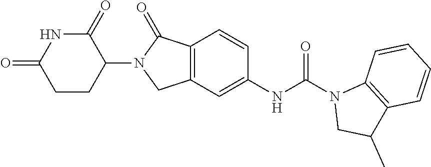 [SMILES: CC1CN(C(=O)Nc2ccc3c(c2)CN(C2CCC(=O)NC2=O)C3=O)c2ccccc21] |
| 120 | 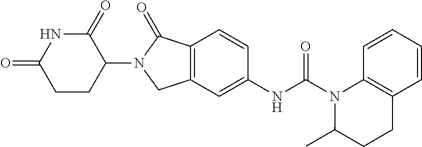 [SMILES: CC1CCc2ccccc2N1C(=O)Nc1ccc2c(c1)CN(C1CCC(=O)NC1=O)C2=O] |
| 121 | 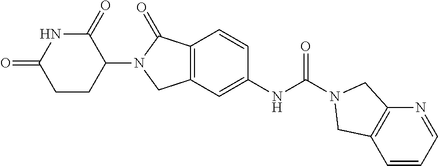 [SMILES: O=C1CCC(N2Cc3cc(NC(=O)N4Cc5cccnc5C4)ccc3C2=O)C(=O)N1] |
| 122 | 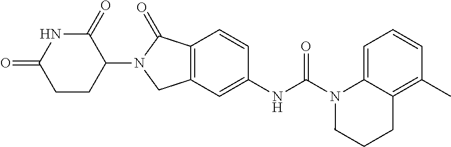 [SMILES: Cc1cccc2c1CCCN2C(=O)Nc1ccc2c(c1)CN(C1CCC(=O)NC1=O)C2=O] |
| 123 | 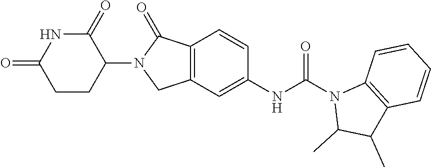 [SMILES: CC1c2ccccc2N(C(=O)Nc2ccc3c(c2)CN(C2CCC(=O)NC2=O)C3=O)C1C] |
| 124 | 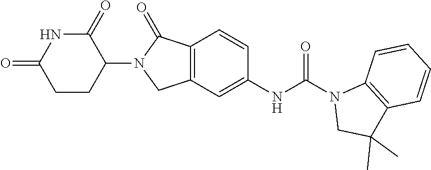 [SMILES: CC1(C)CN(C(=O)Nc2ccc3c(c2)CN(C2CCC(=O)NC2=O)C3=O)c2ccccc21] |
| 125 | 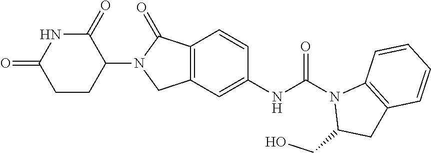 |
| 126 | 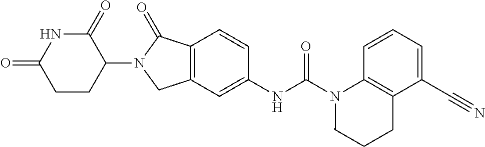 [SMILES: N#Cc1cccc2c1CCCN2C(=O)Nc1ccc2c(c1)CN(C1CCC(=O)NC1=O)C2=O] |
| 127 | 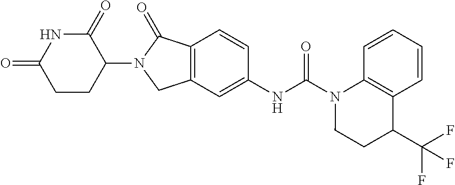 [SMILES: O=C1CCC(N2Cc3cc(NC(=O)N4CCC(C(F)(F)F)c5ccccc54)ccc3C2=O)C(=O)N1] |
| 128 | 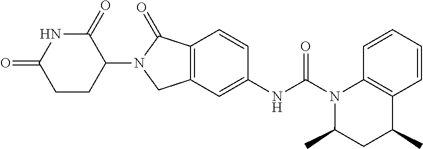 [SMILES: C[C@@H]1C[C@H](C)c2ccccc2N1C(=O)Nc1ccc2c(c1)CN(C1CCC(=O)NC1=O)C2=O] |
| 129 | 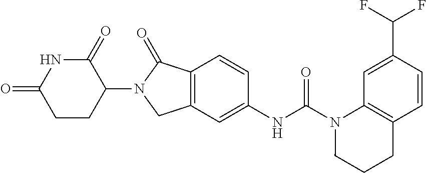 [SMILES: O=C1CCC(N2Cc3cc(NC(=O)N4CCCc5ccc(C(F)F)cc54)ccc3C2=O)C(=O)N1] |
| 130 | 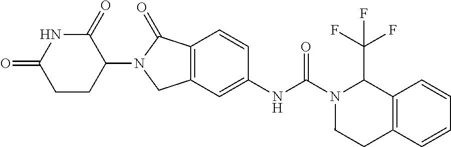 [SMILES: O=C1CCC(N2Cc3cc(NC(=O)N4CCc5ccccc5C4C(F)(F)F)ccc3C2=O)C(=O)N1] |
| 131 | 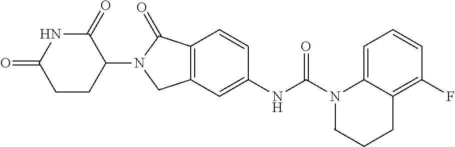 [SMILES: O=C1CCC(N2Cc3cc(NC(=O)N4CCCc5c(F)cccc54)ccc3C2=O)C(=O)N1] |
| 132 | 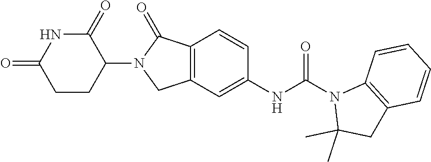 [SMILES: CC1(C)Cc2ccccc2N1C(=O)Nc1ccc2c(c1)CN(C1CCC(=O)NC1=O)C2=O] |
| 133 | 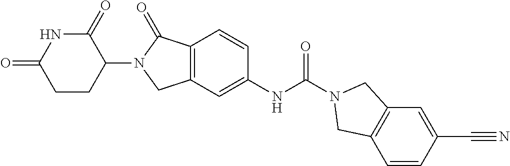 [SMILES: N#Cc1ccc2c(c1)CN(C(=O)Nc1ccc3c(c1)CN(C1CCC(=O)NC1=O)C3=O)C2] |
| 134 | 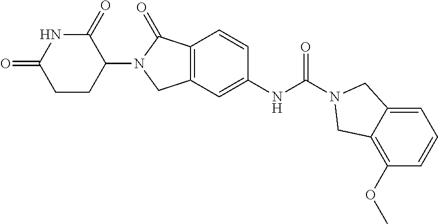 [SMILES: COc1cccc2c1CN(C(=O)Nc1ccc3c(c1)CN(C1CCC(=O)NC1=O)C3=O)C2] |
| 135 | 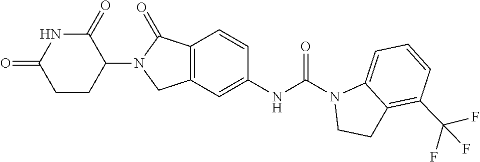 [SMILES: O=C1CCC(N2Cc3cc(NC(=O)N4CCc5c4cccc5C(F)(F)F)ccc3C2=O)C(=O)N1] |
| 136 | 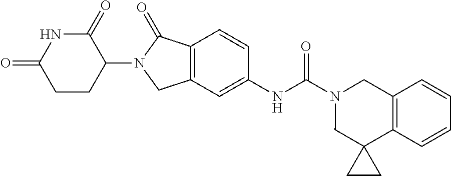 [SMILES: O=C1CCC(N2Cc3cc(NC(=O)N4Cc5ccccc5C5(CC5)C4)ccc3C2=O)C(=O)N1] |
| 137 | 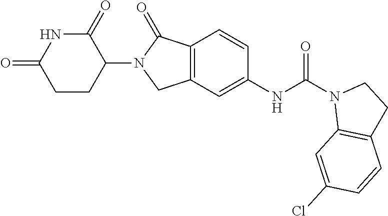 [SMILES: O=C1CCC(N2Cc3cc(NC(=O)N4CCc5ccc(Cl)cc54)ccc3C2=O)C(=O)N1] |
| 138 | 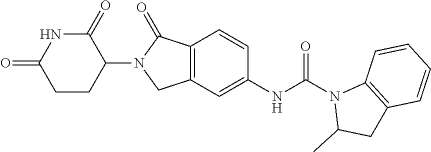 [SMILES: CC1Cc2ccccc2N1C(=O)Nc1ccc2c(c1)CN(C1CCC(=O)NC1=O)C2=O] |
| 139 | 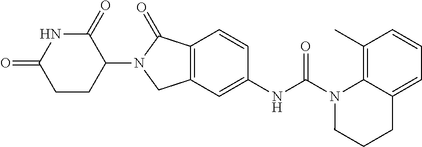 [SMILES: Cc1cccc2c1N(C(=O)Nc1ccc3c(c1)CN(C1CCC(=O)NC1=O)C3=O)CCC2] |
| 140 | 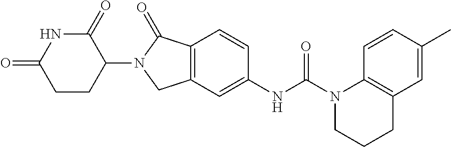 [SMILES: Cc1ccc2c(c1)CCCN2C(=O)Nc1ccc2c(c1)CN(C1CCC(=O)NC1=O)C2=O] |
| 141 | 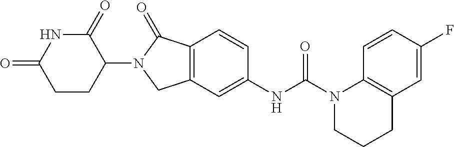 [SMILES: O=C1CCC(N2Cc3cc(NC(=O)N4CCCc5cc(F)ccc54)ccc3C2=O)C(=O)N1] |
| 142 | 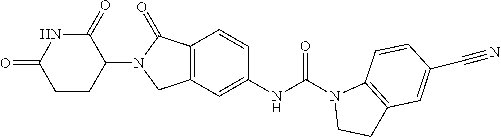 [SMILES: N#Cc1ccc2c(c1)CCN2C(=O)Nc1ccc2c(c1)CN(C1CCC(=O)NC1=O)C2=O] |
| 143 | 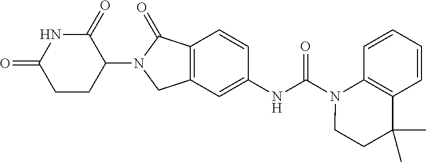 [SMILES: CC1(C)CCN(C(=O)Nc2ccc3c(c2)CN(C2CCC(=O)NC2=O)C3=O)c2ccccc21] |
| 144 | 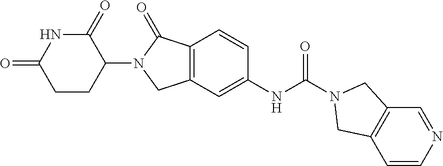 [SMILES: O=C1CCC(N2Cc3cc(NC(=O)N4Cc5ccncc5C4)ccc3C2=O)C(=O)N1] |
| 145 | 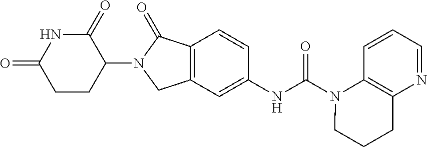 [SMILES: O=C1CCC(N2Cc3cc(NC(=O)N4CCCc5ncccc54)ccc3C2=O)C(=O)N1] |
| 146 | 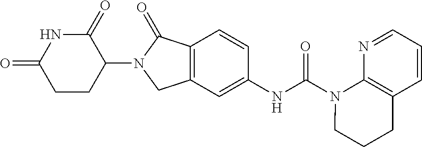 [SMILES: O=C1CCC(N2Cc3cc(NC(=O)N4CCCc5cccnc54)ccc3C2=O)C(=O)N1] |
| 147 | 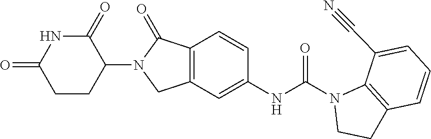 [SMILES: N#Cc1cccc2c1N(C(=O)Nc1ccc3c(c1)CN(C1CCC(=O)NC1=O)C3=O)CC2] |
| 148 | 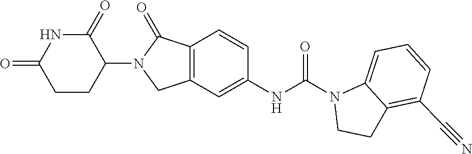 [SMILES: N#Cc1cccc2c1CCN2C(=O)Nc1ccc2c(c1)CN(C1CCC(=O)NC1=O)C2=O] |
| 149 | 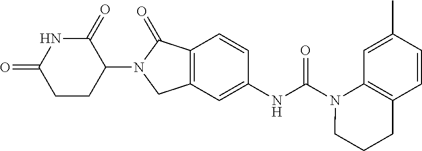 [SMILES: Cc1ccc2c(c1)N(C(=O)Nc1ccc3c(c1)CN(C1CCC(=O)NC1=O)C3=O)CCC2] |
| 150 | 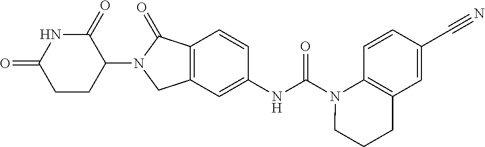 [SMILES: N#Cc1ccc2c(c1)CCCN2C(=O)Nc1ccc2c(c1)CN(C1CCC(=O)NC1=O)C2=O] |
| 151 | 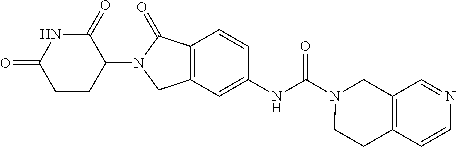 [SMILES: O=C1CCC(N2Cc3cc(NC(=O)N4CCc5ccncc5C4)ccc3C2=O)C(=O)N1] |
| 152 | 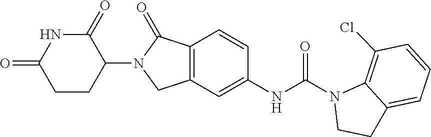 [SMILES: O=C1CCC(N2Cc3cc(NC(=O)N4CCc5cccc(Cl)c54)ccc3C2=O)C(=O)N1] |
| 153 | 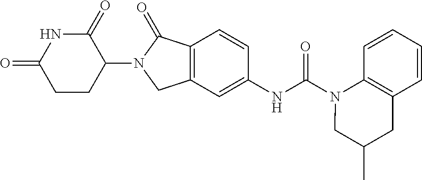 [SMILES: CC1Cc2ccccc2N(C(=O)Nc2ccc3c(c2)CN(C2CCC(=O)NC2=O)C3=O)C1] |
| 154 | 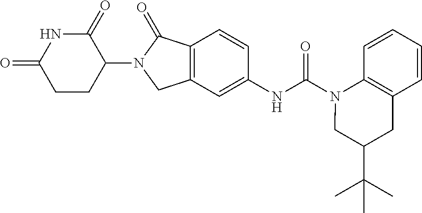 [SMILES: CC(C)(C)C1Cc2ccccc2N(C(=O)Nc2ccc3c(c2)CN(C2CCC(=O)NC2=O)C3=O)C1] |
| 155 | 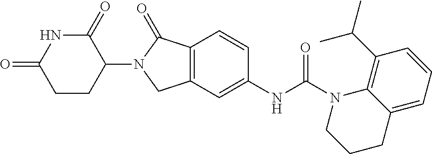 [SMILES: CC(C)c1cccc2c1N(C(=O)Nc1ccc3c(c1)CN(C1CCC(=O)NC1=O)C3=O)CCC2] |
| 156 | 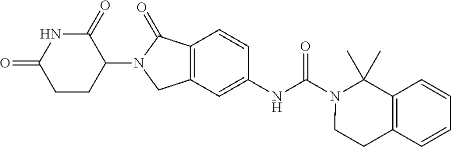 [SMILES: CC1(C)c2ccccc2CCN1C(=O)Nc1ccc2c(c1)CN(C1CCC(=O)NC1=O)C2=O] |
| 157 | 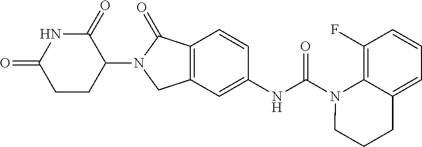 [SMILES: O=C1CCC(N2Cc3cc(NC(=O)N4CCCc5cccc(F)c54)ccc3C2=O)C(=O)N1] |
| 158 | 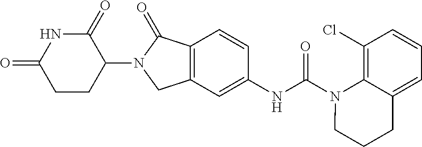 [SMILES: O=C1CCC(N2Cc3cc(NC(=O)N4CCCc5cccc(Cl)c54)ccc3C2=O)C(=O)N1] |
| 159 | 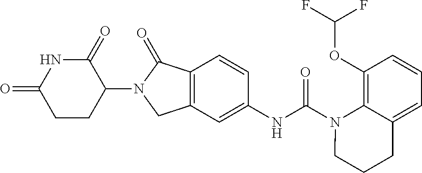 [SMILES: O=C1CCC(N2Cc3cc(NC(=O)N4CCCc5cccc(OC(F)F)c54)ccc3C2=O)C(=O)N1] |
| 160 | 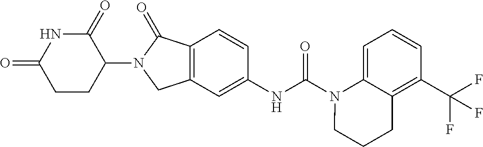 [SMILES: O=C1CCC(N2Cc3cc(NC(=O)N4CCCc5c4cccc5C(F)(F)F)ccc3C2=O)C(=O)N1] |
| 161 | 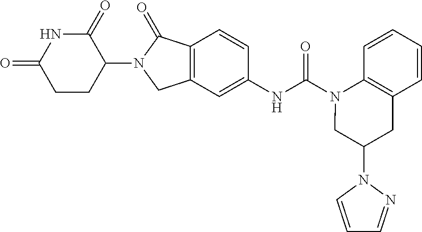 [SMILES: O=C1CCC(N2Cc3cc(NC(=O)N4CC(n5cccn5)Cc5ccccc54)ccc3C2=O)C(=O)N1] |
| 162 | 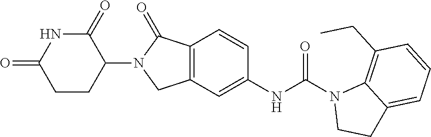 [SMILES: CCc1cccc2c1N(C(=O)Nc1ccc3c(c1)CN(C1CCC(=O)NC1=O)C3=O)CC2] |
| 163 | 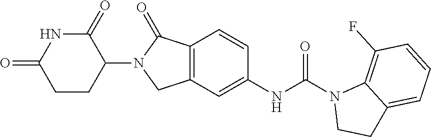 [SMILES: O=C1CCC(N2Cc3cc(NC(=O)N4CCc5cccc(F)c54)ccc3C2=O)C(=O)N1] |
| 164 | 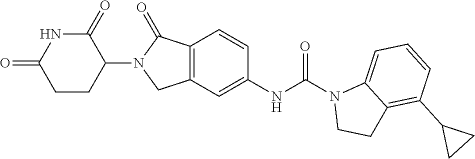 [SMILES: O=C1CCC(N2Cc3cc(NC(=O)N4CCc5c(C6CC6)cccc54)ccc3C2=O)C(=O)N1] |
| 165 | 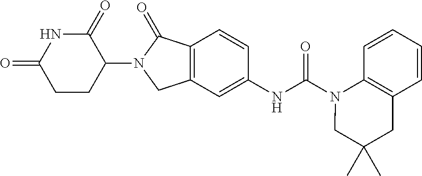 [SMILES: CC1(C)Cc2ccccc2N(C(=O)Nc2ccc3c(c2)CN(C2CCC(=O)NC2=O)C3=O)C1] |
| 166 | 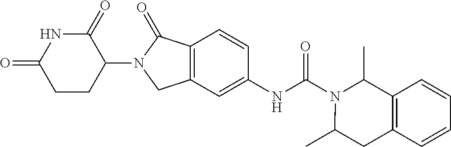 [SMILES: CC1Cc2ccccc2C(C)N1C(=O)Nc1ccc2c(c1)CN(C1CCC(=O)NC1=O)C2=O] |
| 167 | 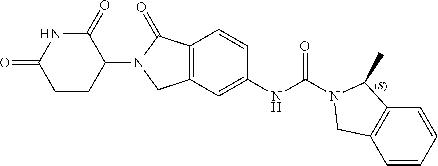 |
| 168 | 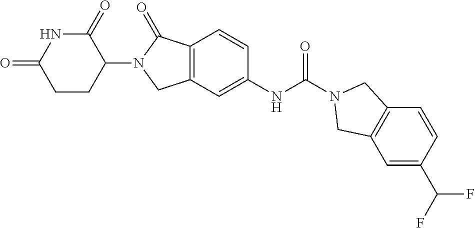 [SMILES: O=C1CCC(N2Cc3cc(NC(=O)N4Cc5ccc(C(F)F)cc5C4)ccc3C2=O)C(=O)N1] |
| 169 | 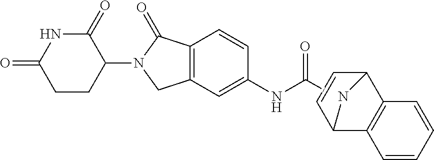 |
| 170 | 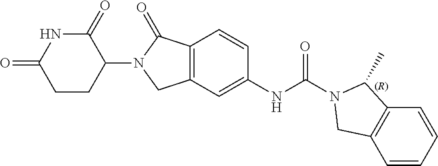 |
| 171 | 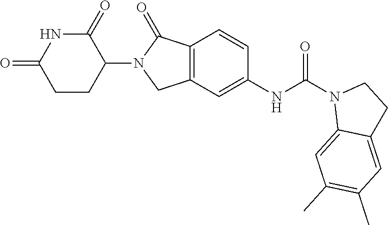 [SMILES: Cc1cc2c(cc1C)N(C(=O)Nc1ccc3c(c1)CN(C1CCC(=O)NC1=O)C3=O)CC2] |
| 172 | 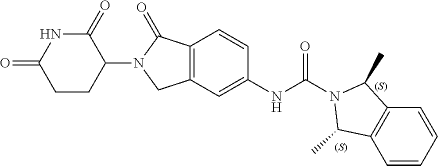 |
| 173 | 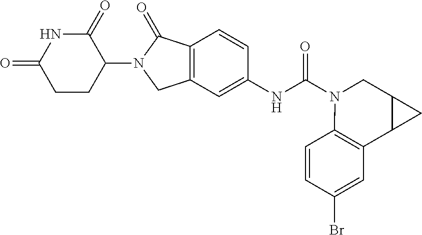 [SMILES: O=C1CCC(N2Cc3cc(NC(=O)N4CC5CC5c5cc(Br)ccc54)ccc3C2=O)C(=O)N1] |
| 174 | 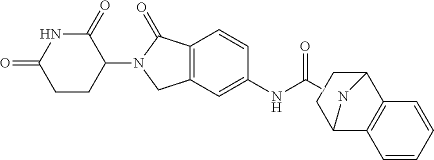 |
| 175 | 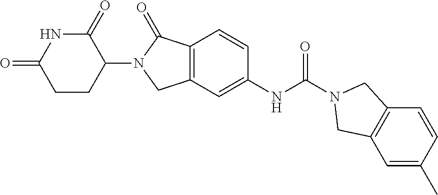 [SMILES: Cc1ccc2c(c1)CN(C(=O)Nc1ccc3c(c1)CN(C1CCC(=O)NC1=O)C3=O)C2] |
| 176 | 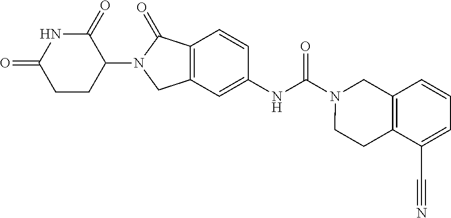 [SMILES: N#Cc1cccc2c1CCN(C(=O)Nc1ccc3c(c1)CN(C1CCC(=O)NC1=O)C3=O)C2] |
| 177 | 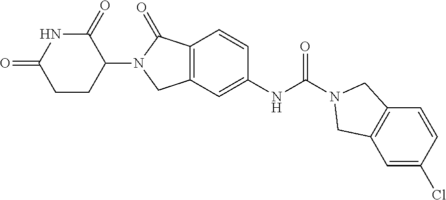 [SMILES: O=C1CCC(N2Cc3cc(NC(=O)N4Cc5ccc(Cl)cc5C4)ccc3C2=O)C(=O)N1] |
| 178 | 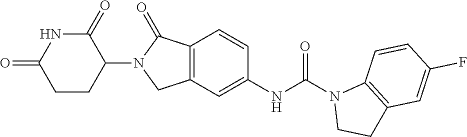 [SMILES: O=C1CCC(N2Cc3cc(NC(=O)N4CCc5cc(F)ccc54)ccc3C2=O)C(=O)N1] |
| 179 | 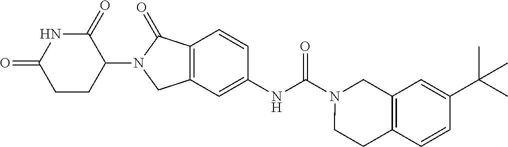 [SMILES: CC(C)(C)c1ccc2c(c1)CN(C(=O)Nc1ccc3c(c1)CN(C1CCC(=O)NC1=O)C3=O)CC2] |
| 180 | 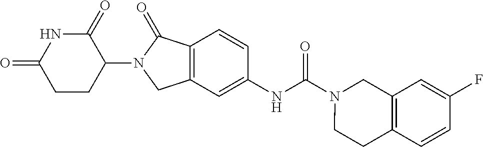 [SMILES: O=C1CCC(N2Cc3cc(NC(=O)N4CCc5ccc(F)cc5C4)ccc3C2=O)C(=O)N1] |
| 181 | 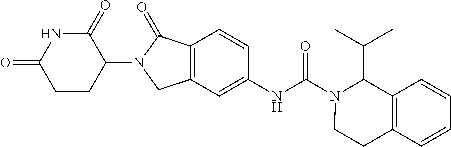 [SMILES: CC(C)C1c2ccccc2CCN1C(=O)Nc1ccc2c(c1)CN(C1CCC(=O)NC1=O)C2=O] |
| 182 | 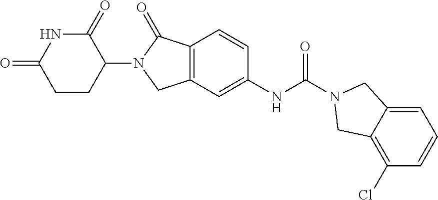 [SMILES: O=C1CCC(N2Cc3cc(NC(=O)N4Cc5cccc(Cl)c5C4)ccc3C2=O)C(=O)N1] |
| 183 | 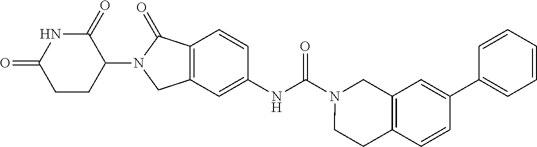 [SMILES: O=C1CCC(N2Cc3cc(NC(=O)N4CCc5ccc(-c6ccccc6)cc5C4)ccc3C2=O)C(=O)N1] |
| 184 | 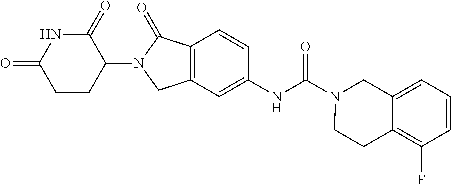 [SMILES: O=C1CCC(N2Cc3cc(NC(=O)N4CCc5c(F)cccc5C4)ccc3C2=O)C(=O)N1] |
| 185 | 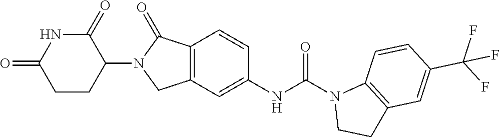 [SMILES: O=C1CCC(N2Cc3cc(NC(=O)N4CCc5cc(C(F)(F)F)ccc54)ccc3C2=O)C(=O)N1] |
| 186 | 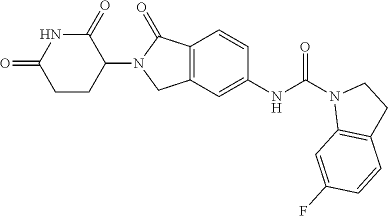 [SMILES: O=C1CCC(N2Cc3cc(NC(=O)N4CCc5ccc(F)cc54)ccc3C2=O)C(=O)N1] |
| 187 | 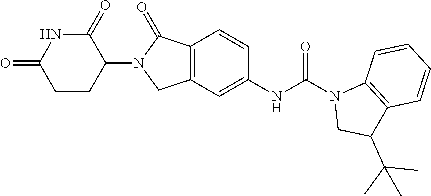 [SMILES: CC(C)(C)C1CN(C(=O)Nc2ccc3c(c2)CN(C2CCC(=O)NC2=O)C3=O)c2ccccc21] |
| 188 | 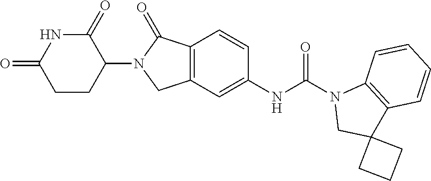 [SMILES: O=C1CCC(N2Cc3cc(NC(=O)N4CC5(CCC5)c5ccccc54)ccc3C2=O)C(=O)N1] |
| 189 | 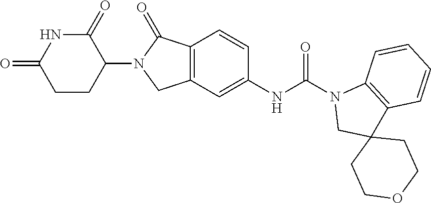 [SMILES: O=C1CCC(N2Cc3cc(NC(=O)N4CC5(CCOCC5)c5ccccc54)ccc3C2=O)C(=O)N1] |
| 190 | 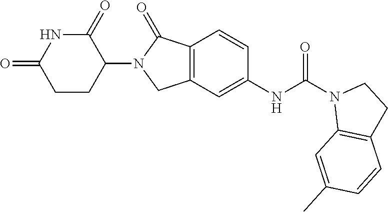 [SMILES: Cc1ccc2c(c1)N(C(=O)Nc1ccc3c(c1)CN(C1CCC(=O)NC1=O)C3=O)CC2] |
| 191 | 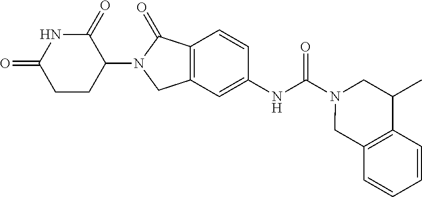 [SMILES: CC1CN(C(=O)Nc2ccc3c(c2)CN(C2CCC(=O)NC2=O)C3=O)Cc2ccccc21] |
| 192 | 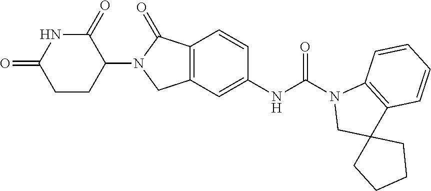 [SMILES: O=C1CCC(N2Cc3cc(NC(=O)N4CC5(CCCC5)c5ccccc54)ccc3C2=O)C(=O)N1] |
| 193 | 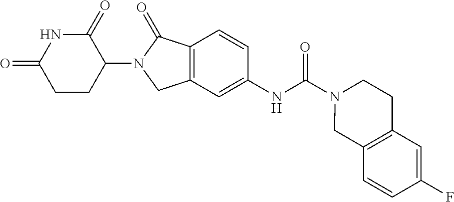 [SMILES: O=C1CCC(N2Cc3cc(NC(=O)N4CCc5cc(F)ccc5C4)ccc3C2=O)C(=O)N1] |
| 194 | 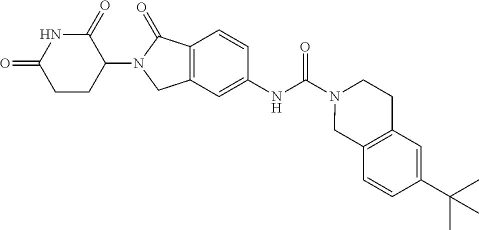 [SMILES: CC(C)(C)c1ccc2c(c1)CCN(C(=O)Nc1ccc3c(c1)CN(C1CCC(=O)NC1=O)C3=O)C2] |
| 195 | 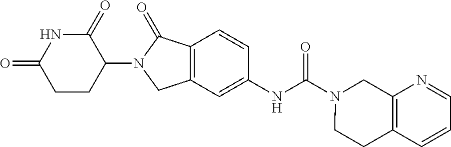 [SMILES: O=C1CCC(N2Cc3cc(NC(=O)N4CCc5cccnc5C4)ccc3C2=O)C(=O)N1] |
| 196 | 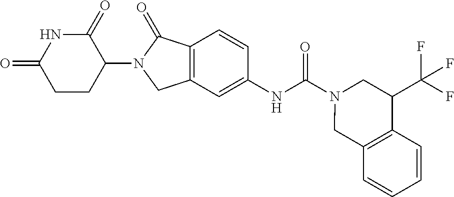 [SMILES: O=C1CCC(N2Cc3cc(NC(=O)N4Cc5ccccc5C(C(F)(F)F)C4)ccc3C2=O)C(=O)N1] |
| 197 | 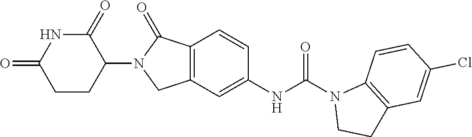 [SMILES: O=C1CCC(N2Cc3cc(NC(=O)N4CCc5cc(Cl)ccc54)ccc3C2=O)C(=O)N1] |
| 198 | 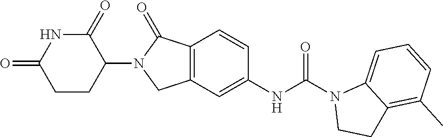 [SMILES: Cc1cccc2c1CCN2C(=O)Nc1ccc2c(c1)CN(C1CCC(=O)NC1=O)C2=O] |
| 199 | 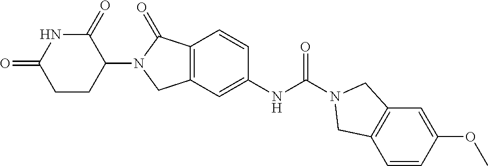 [SMILES: COc1ccc2c(c1)CN(C(=O)Nc1ccc3c(c1)CN(C1CCC(=O)NC1=O)C3=O)C2] |
| 200 | 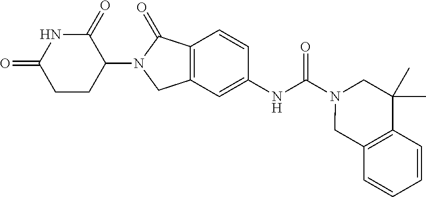 [SMILES: CC1(C)CN(C(=O)Nc2ccc3c(c2)CN(C2CCC(=O)NC2=O)C3=O)Cc2ccccc21] |
| 201 | 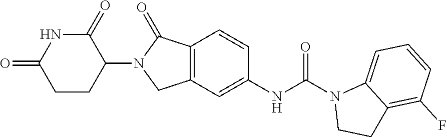 [SMILES: O=C1CCC(N2Cc3cc(NC(=O)N4CCc5c(F)cccc54)ccc3C2=O)C(=O)N1] |
| 202 | 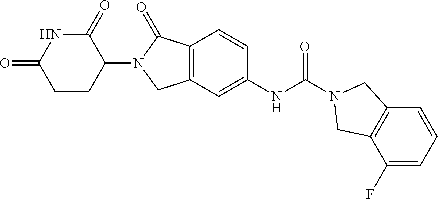 [SMILES: O=C1CCC(N2Cc3cc(NC(=O)N4Cc5cccc(F)c5C4)ccc3C2=O)C(=O)N1] |
| 203 | 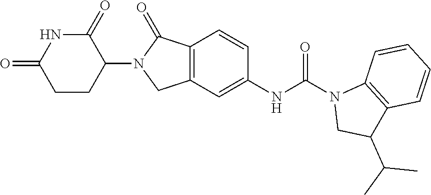 [SMILES: CC(C)C1CN(C(=O)Nc2ccc3c(c2)CN(C2CCC(=O)NC2=O)C3=O)c2ccccc21] |
| 204 | 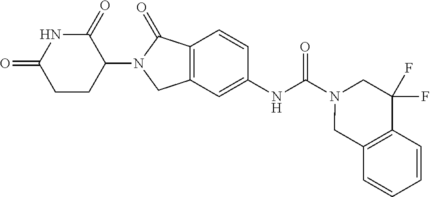 [SMILES: O=C1CCC(N2Cc3cc(NC(=O)N4Cc5ccccc5C(F)(F)C4)ccc3C2=O)C(=O)N1] |
| 205 | 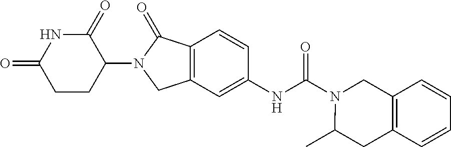 [SMILES: CC1Cc2ccccc2CN1C(=O)Nc1ccc2c(c1)CN(C1CCC(=O)NC1=O)C2=O] |
| 206 | 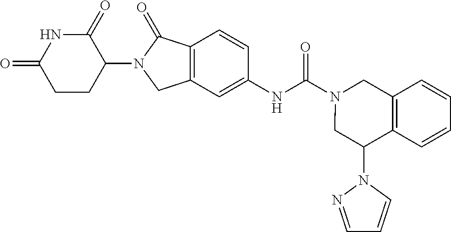 [SMILES: O=C1CCC(N2Cc3cc(NC(=O)N4Cc5ccccc5C(n5cccn5)C4)ccc3C2=O)C(=O)N1] |
| 207 | 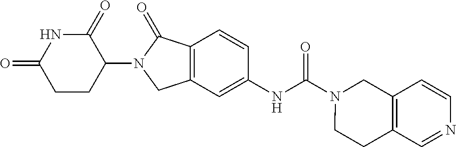 [SMILES: O=C1CCC(N2Cc3cc(NC(=O)N4CCc5cnccc5C4)ccc3C2=O)C(=O)N1] |
| 208 | 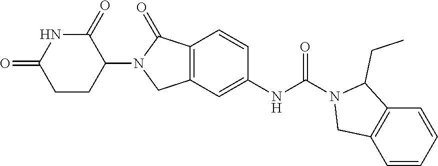 [SMILES: CCC1c2ccccc2CN1C(=O)Nc1ccc2c(c1)CN(C1CCC(=O)NC1=O)C2=O] |
| 209 | 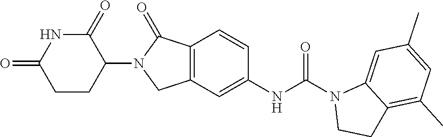 [SMILES: Cc1cc(C)c2c(c1)N(C(=O)Nc1ccc3c(c1)CN(C1CCC(=O)NC1=O)C3=O)CC2] |
| 210 | 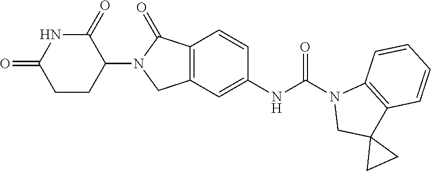 [SMILES: O=C1CCC(N2Cc3cc(NC(=O)N4CC5(CC5)c5ccccc54)ccc3C2=O)C(=O)N1] |
| 211 | 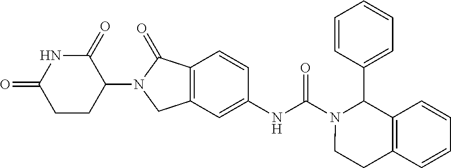 [SMILES: O=C1CCC(N2Cc3cc(NC(=O)N4CCc5ccccc5C4c4ccccc4)ccc3C2=O)C(=O)N1] |
| 212 | 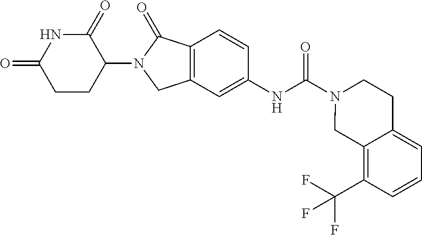 [SMILES: O=C1CCC(N2Cc3cc(NC(=O)N4CCc5cccc(C(F)(F)F)c5C4)ccc3C2=O)C(=O)N1] |
| 213 | 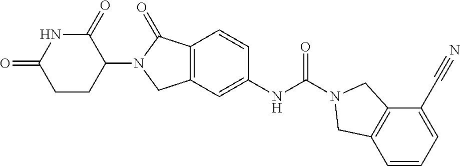 [SMILES: N#Cc1cccc2c1CN(C(=O)Nc1ccc3c(c1)CN(C1CCC(=O)NC1=O)C3=O)C2] |
| 214 | 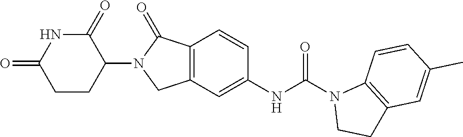 [SMILES: Cc1ccc2c(c1)CCN2C(=O)Nc1ccc2c(c1)CN(C1CCC(=O)NC1=O)C2=O] |
| 215 | 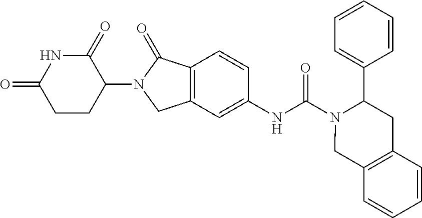 [SMILES: O=C1CCC(N2Cc3cc(NC(=O)N4Cc5ccccc5CC4c4ccccc4)ccc3C2=O)C(=O)N1] |
| 216 | 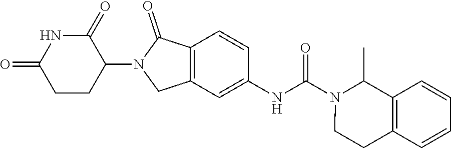 [SMILES: CC1c2ccccc2CCN1C(=O)Nc1ccc2c(c1)CN(C1CCC(=O)NC1=O)C2=O] |
| 217 | 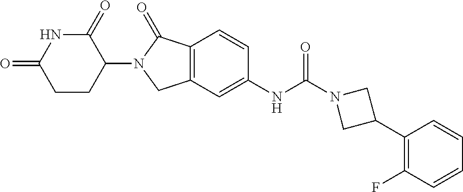 [SMILES: O=C1CCC(N2Cc3cc(NC(=O)N4CC(c5ccccc5F)C4)ccc3C2=O)C(=O)N1] |
| 218 | 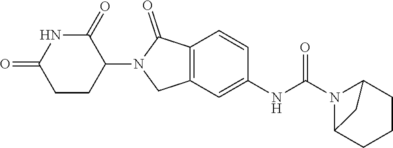 [SMILES: O=C1CCC(N2Cc3cc(NC(=O)N4C5CCCC4C5)ccc3C2=O)C(=O)N1] |
| 219 | 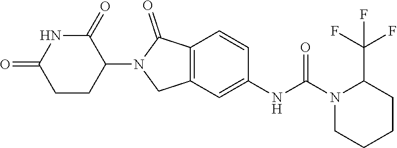 [SMILES: O=C1CCC(N2Cc3cc(NC(=O)N4CCCCC4C(F)(F)F)ccc3C2=O)C(=O)N1] |
| 220 | 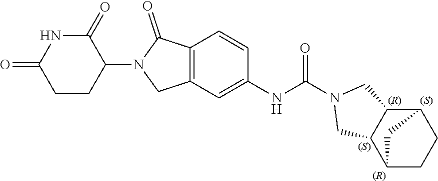 |
| 221 | 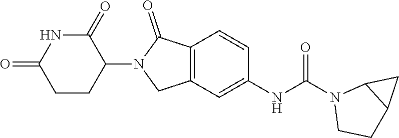 [SMILES: O=C1CCC(N2Cc3cc(NC(=O)N4CCC5CC54)ccc3C2=O)C(=O)N1] |
| 222 | 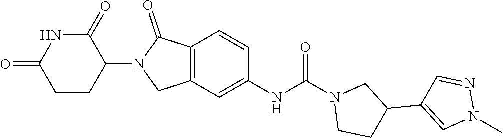 [SMILES: Cn1cc(C2CCN(C(=O)Nc3ccc4c(c3)CN(C3CCC(=O)NC3=O)C4=O)C2)cn1] |
| 223 | 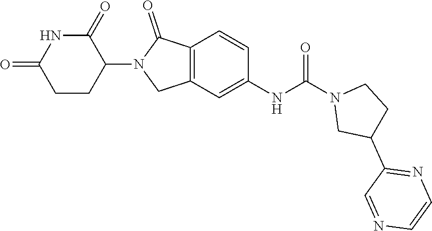 [SMILES: O=C1CCC(N2Cc3cc(NC(=O)N4CCC(c5cnccn5)C4)ccc3C2=O)C(=O)N1] |
| 224 | 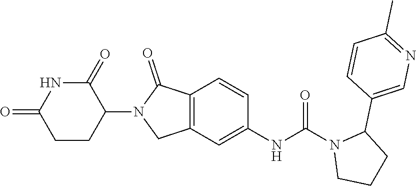 [SMILES: Cc1ccc(C2CCCN2C(=O)Nc2ccc3c(c2)CN(C2CCC(=O)NC2=O)C3=O)cn1] |
| 225 | 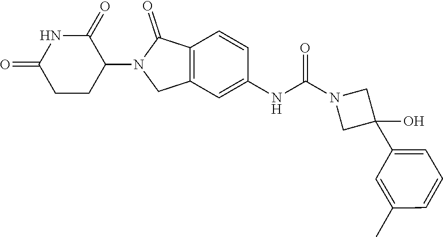 [SMILES: Cc1cccc(C2(O)CN(C(=O)Nc3ccc4c(c3)CN(C3CCC(=O)NC3=O)C4=O)C2)c1] |
| 226 | 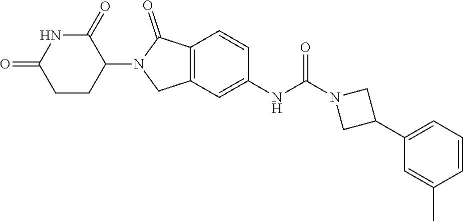 [SMILES: Cc1cccc(C2CN(C(=O)Nc3ccc4c(c3)CN(C3CCC(=O)NC3=O)C4=O)C2)c1] |
| 227 | 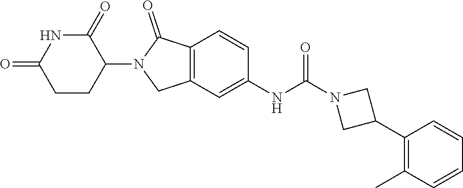 [SMILES: Cc1ccccc1C1CN(C(=O)Nc2ccc3c(c2)CN(C2CCC(=O)NC2=O)C3=O)C1] |
| 228 | 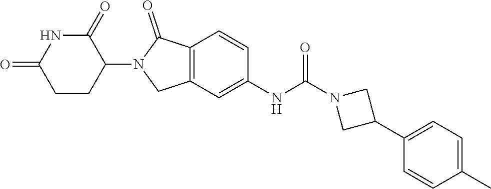 [SMILES: Cc1ccc(C2CN(C(=O)Nc3ccc4c(c3)CN(C3CCC(=O)NC3=O)C4=O)C2)cc1] |
| 229 | 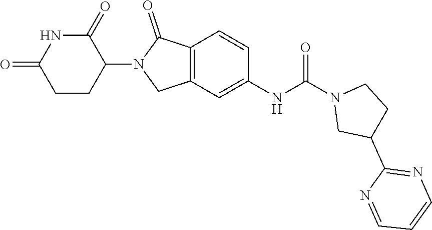 [SMILES: O=C1CCC(N2Cc3cc(NC(=O)N4CCC(c5ncccn5)C4)ccc3C2=O)C(=O)N1] |
| 230 | 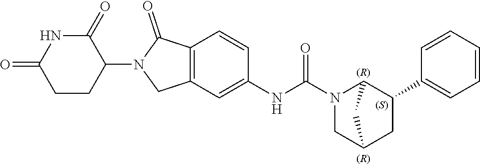 |
| 231 | 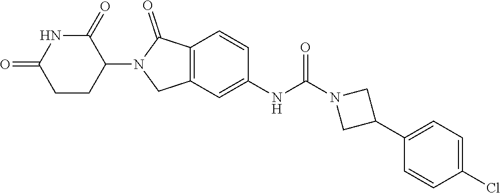 [SMILES: O=C1CCC(N2Cc3cc(NC(=O)N4CC(c5ccc(Cl)cc5)C4)ccc3C2=O)C(=O)N1] |
| 232 | 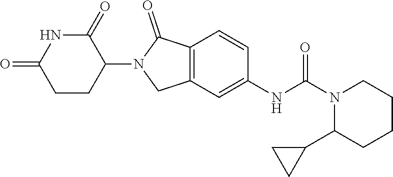 [SMILES: O=C1CCC(N2Cc3cc(NC(=O)N4CCCCC4C4CC4)ccc3C2=O)C(=O)N1] |
| 233 | 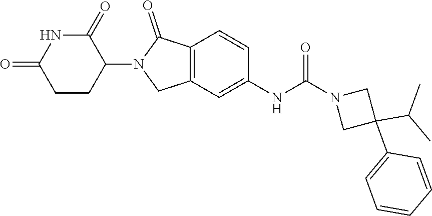 [SMILES: CC(C)C1(c2ccccc2)CN(C(=O)Nc2ccc3c(c2)CN(C2CCC(=O)NC2=O)C3=O)C1] |
| 234 | 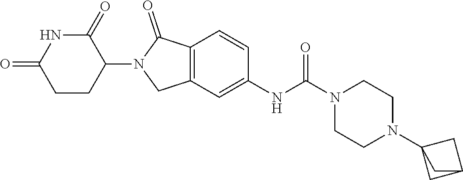 [SMILES: O=C1CCC(N2Cc3cc(NC(=O)N4CCN(C56CC(C5)C6)CC4)ccc3C2=O)C(=O)N1] |
| 235 | 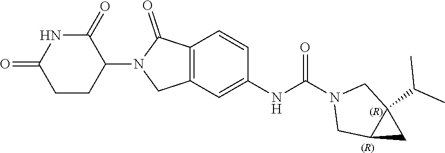 |
| 236 | 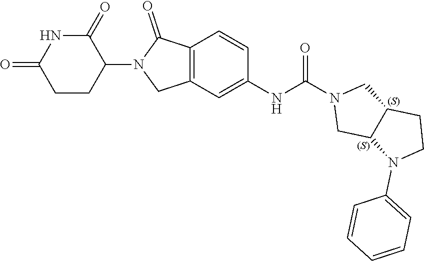 |
| 237 | 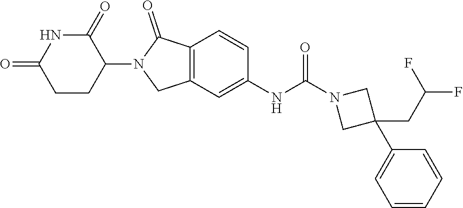 [SMILES: O=C1CCC(N2Cc3cc(NC(=O)N4CC(CC(F)F)(c5ccccc5)C4)ccc3C2=O)C(=O)N1] |
| 238 | 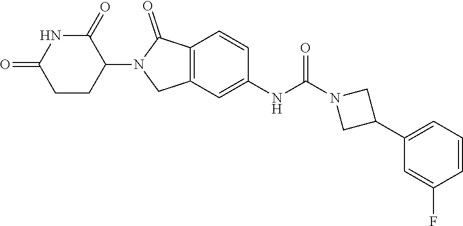 [SMILES: O=C1CCC(N2Cc3cc(NC(=O)N4CC(c5cccc(F)c5)C4)ccc3C2=O)C(=O)N1] |
| 239 | 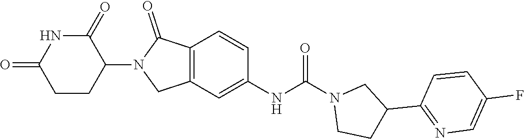 [SMILES: O=C1CCC(N2Cc3cc(NC(=O)N4CCC(c5ccc(F)cn5)C4)ccc3C2=O)C(=O)N1] |
| 240 | 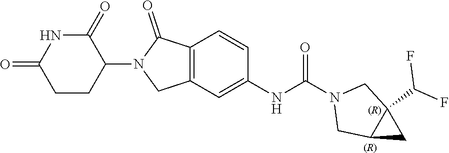 |
| 241 | 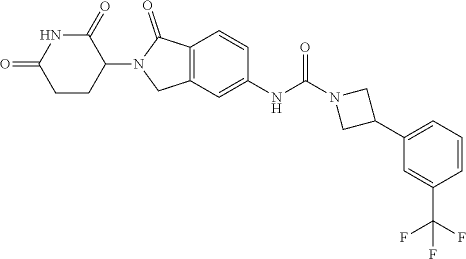 [SMILES: O=C1CCC(N2Cc3cc(NC(=O)N4CC(c5cccc(C(F)(F)F)c5)C4)ccc3C2=O)C(=O)N1] |
| 242 | 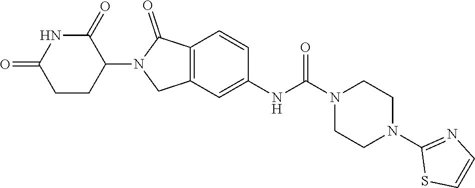 [SMILES: O=C1CCC(N2Cc3cc(NC(=O)N4CCN(c5nccs5)CC4)ccc3C2=O)C(=O)N1] |
| 243 | 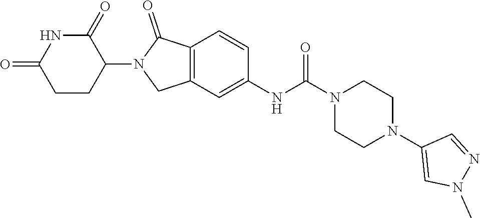 [SMILES: Cn1cc(N2CCN(C(=O)Nc3ccc4c(c3)CN(C3CCC(=O)NC3=O)C4=O)CC2)cn1] |
| 244 | 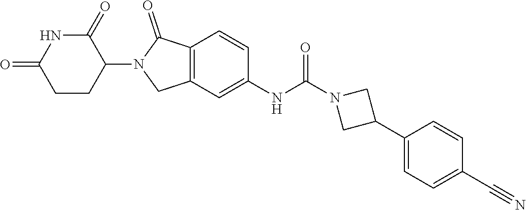 [SMILES: N#Cc1ccc(C2CN(C(=O)Nc3ccc4c(c3)CN(C3CCC(=O)NC3=O)C4=O)C2)cc1] |
| 245 | 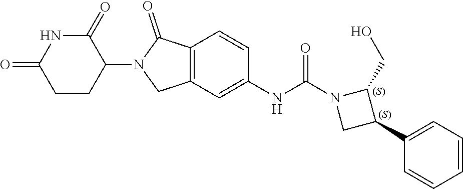 |
| 246 | 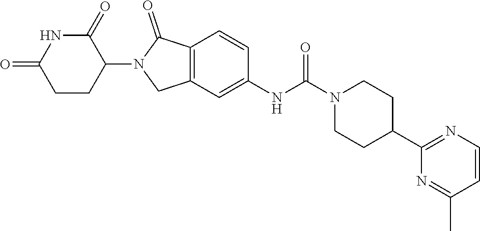 [SMILES: Cc1ccnc(C2CCN(C(=O)Nc3ccc4c(c3)CN(C3CCC(=O)NC3=O)C4=O)CC2)n1] |
| 247 | 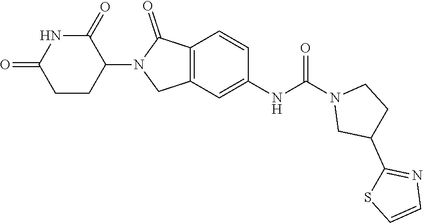 [SMILES: O=C1CCC(N2Cc3cc(NC(=O)N4CCC(c5nccs5)C4)ccc3C2=O)C(=O)N1] |
| 248 | 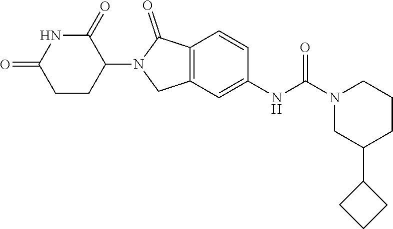 [SMILES: O=C1CCC(N2Cc3cc(NC(=O)N4CCCC(C5CCC5)C4)ccc3C2=O)C(=O)N1] |
| 249 | 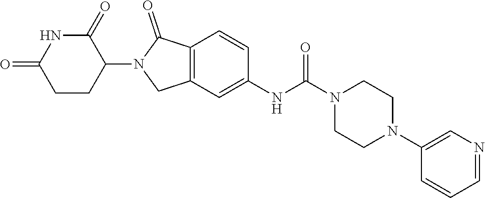 [SMILES: O=C1CCC(N2Cc3cc(NC(=O)N4CCN(c5cccnc5)CC4)ccc3C2=O)C(=O)N1] |
| 250 | 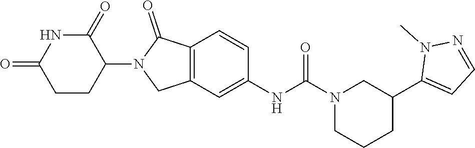 [SMILES: Cn1nccc1C1CCCN(C(=O)Nc2ccc3c(c2)CN(C2CCC(=O)NC2=O)C3=O)C1] |
| 251 | 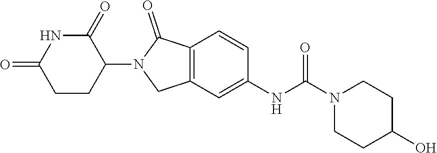 [SMILES: O=C1CCC(N2Cc3cc(NC(=O)N4CCC(O)CC4)ccc3C2=O)C(=O)N1] |
| 252 | 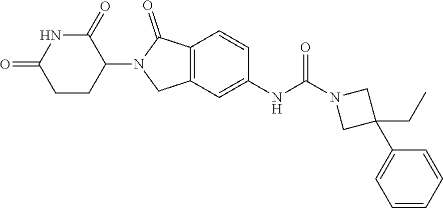 [SMILES: CCC1(c2ccccc2)CN(C(=O)Nc2ccc3c(c2)CN(C2CCC(=O)NC2=O)C3=O)C1] |
| 253 | 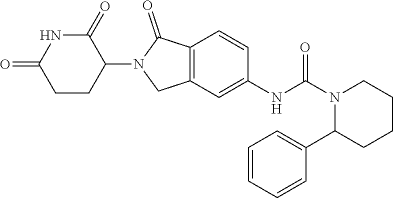 [SMILES: O=C1CCC(N2Cc3cc(NC(=O)N4CCCCC4c4ccccc4)ccc3C2=O)C(=O)N1] |
| 254 | 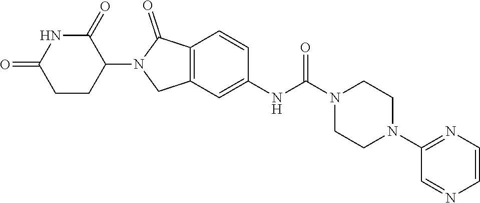 [SMILES: O=C1CCC(N2Cc3cc(NC(=O)N4CCN(c5cnccn5)CC4)ccc3C2=O)C(=O)N1] |
| 255 | 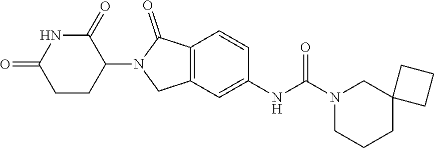 [SMILES: O=C1CCC(N2Cc3cc(NC(=O)N4CCCC5(CCC5)C4)ccc3C2=O)C(=O)N1] |
| 256 | 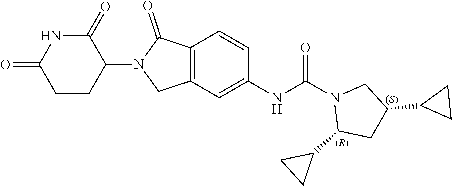 |
| 257 | 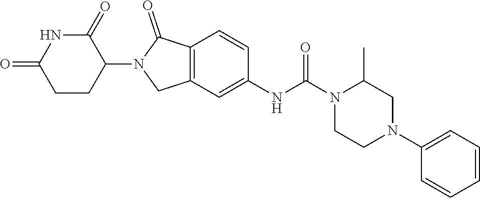 [SMILES: CC1CN(c2ccccc2)CCN1C(=O)Nc1ccc2c(c1)CN(C1CCC(=O)NC1=O)C2=O] |
| 258 | 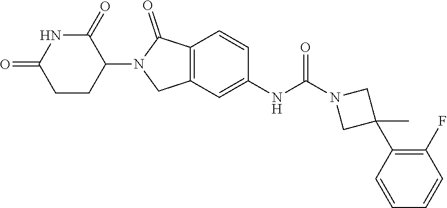 [SMILES: CC1(c2ccccc2F)CN(C(=O)Nc2ccc3c(c2)CN(C2CCC(=O)NC2=O)C3=O)C1] |
| 259 | 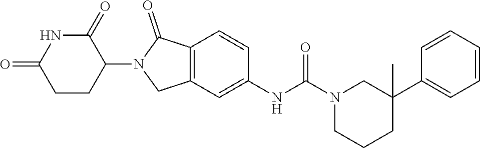 [SMILES: CC1(c2ccccc2)CCCN(C(=O)Nc2ccc3c(c2)CN(C2CCC(=O)NC2=O)C3=O)C1] |
| 260 | 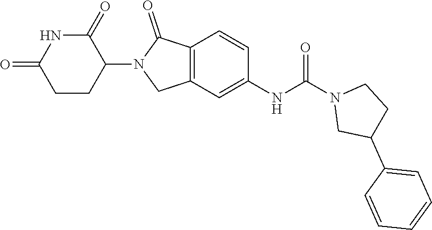 [SMILES: O=C1CCC(N2Cc3cc(NC(=O)N4CCC(c5ccccc5)C4)ccc3C2=O)C(=O)N1] |
| 261 | 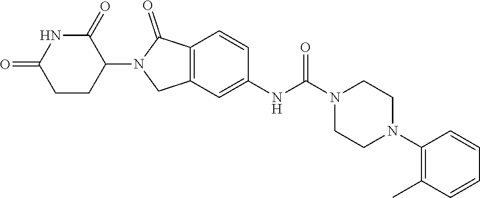 [SMILES: Cc1ccccc1N1CCN(C(=O)Nc2ccc3c(c2)CN(C2CCC(=O)NC2=O)C3=O)CC1] |
| 262 | 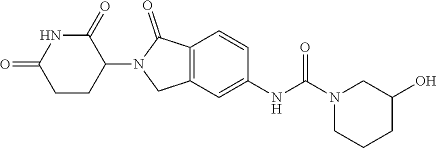 [SMILES: O=C1CCC(N2Cc3cc(NC(=O)N4CCCC(O)C4)ccc3C2=O)C(=O)N1] |
| 263 | 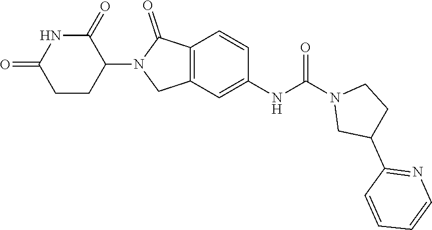 [SMILES: O=C1CCC(N2Cc3cc(NC(=O)N4CCC(c5ccccn5)C4)ccc3C2=O)C(=O)N1] |
| 264 | 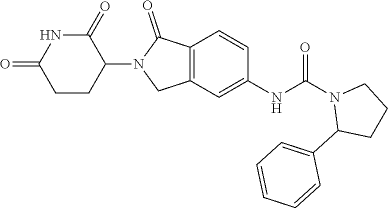 [SMILES: O=C1CCC(N2Cc3cc(NC(=O)N4CCCC4c4ccccc4)ccc3C2=O)C(=O)N1] |
| 265 | 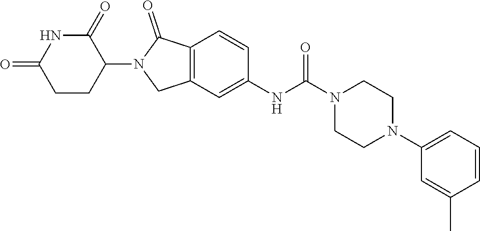 [SMILES: Cc1cccc(N2CCN(C(=O)Nc3ccc4c(c3)CN(C3CCC(=O)NC3=O)C4=O)CC2)c1] |
| 266 | 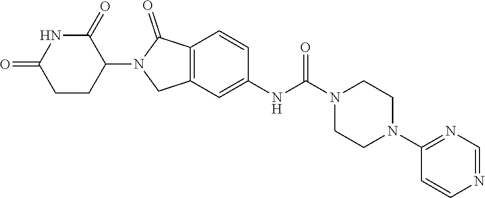 [SMILES: O=C1CCC(N2Cc3cc(NC(=O)N4CCN(c5ccncn5)CC4)ccc3C2=O)C(=O)N1] |
| 267 | 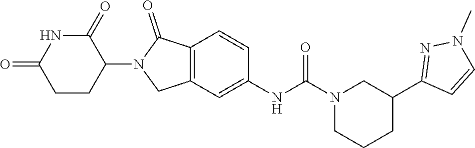 [SMILES: Cn1ccc(C2CCCN(C(=O)Nc3ccc4c(c3)CN(C3CCC(=O)NC3=O)C4=O)C2)n1] |
| 268 | 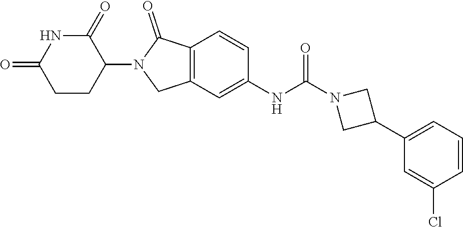 [SMILES: O=C1CCC(N2Cc3cc(NC(=O)N4CC(c5cccc(Cl)c5)C4)ccc3C2=O)C(=O)N1] |
| 269 | 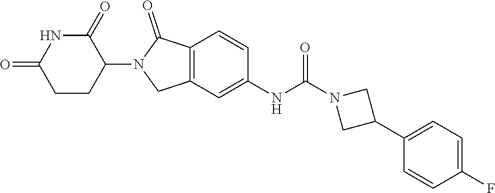 [SMILES: O=C1CCC(N2Cc3cc(NC(=O)N4CC(c5ccc(F)cc5)C4)ccc3C2=O)C(=O)N1] |
| 270 | 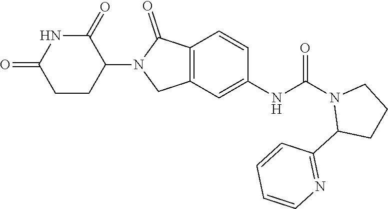 [SMILES: O=C1CCC(N2Cc3cc(NC(=O)N4CCCC4c4ccccn4)ccc3C2=O)C(=O)N1] |
| 271 | 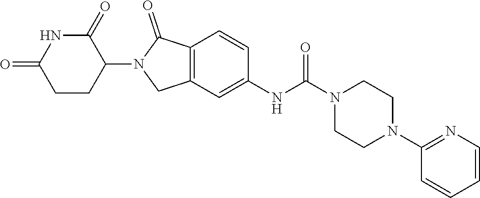 [SMILES: O=C1CCC(N2Cc3cc(NC(=O)N4CCN(c5ccccn5)CC4)ccc3C2=O)C(=O)N1] |
| 272 | 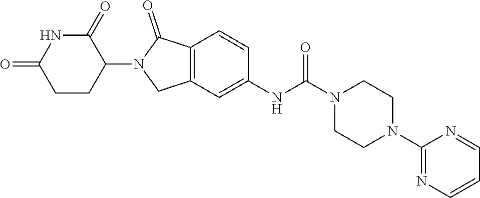 [SMILES: O=C1CCC(N2Cc3cc(NC(=O)N4CCN(c5ncccn5)CC4)ccc3C2=O)C(=O)N1] |
| 273 | 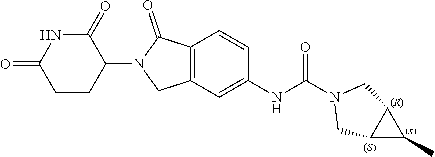 |
| 274 | 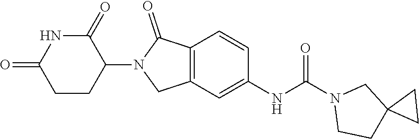 [SMILES: O=C1CCC(N2Cc3cc(NC(=O)N4CCC5(CC5)C4)ccc3C2=O)C(=O)N1] |
| 275 | 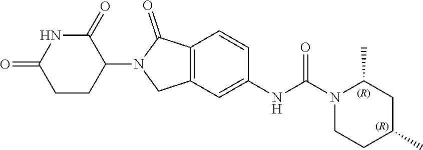 |
| 276 | 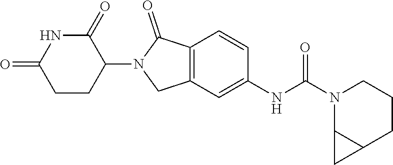 [SMILES: O=C1CCC(N2Cc3cc(NC(=O)N4CCCC5CC54)ccc3C2=O)C(=O)N1] |
| 277 | 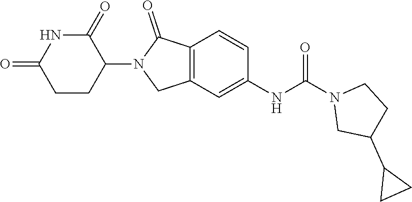 [SMILES: O=C1CCC(N2Cc3cc(NC(=O)N4CCC(C5CC5)C4)ccc3C2=O)C(=O)N1] |
| 278 | 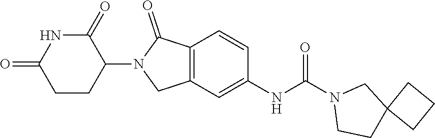 [SMILES: O=C1CCC(N2Cc3cc(NC(=O)N4CCC5(CCC5)C4)ccc3C2=O)C(=O)N1] |
| 279 | 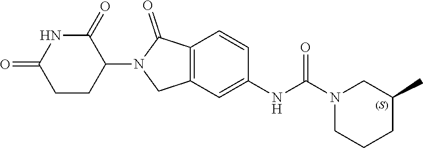 |
| 280 | 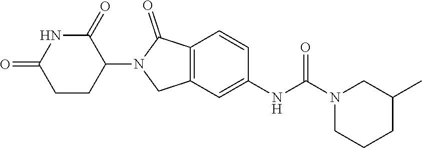 [SMILES: CC1CCCN(C(=O)Nc2ccc3c(c2)CN(C2CCC(=O)NC2=O)C3=O)C1] |
| 281 | 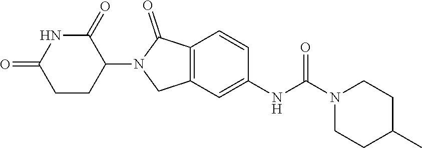 [SMILES: CC1CCN(C(=O)Nc2ccc3c(c2)CN(C2CCC(=O)NC2=O)C3=O)CC1] |
| 282 | 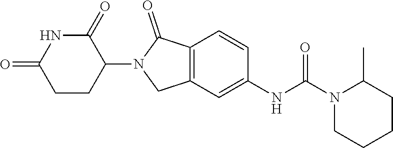 [SMILES: CC1CCCCN1C(=O)Nc1ccc2c(c1)CN(C1CCC(=O)NC1=O)C2=O] |
| 283 | 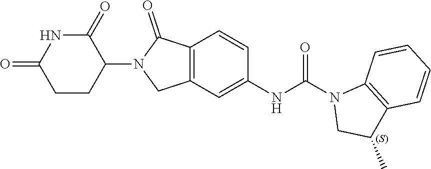 |
| 284 | 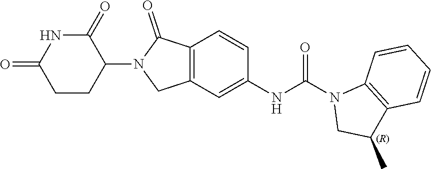 |
| 285 | 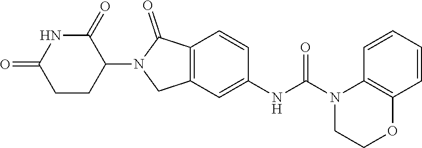 [SMILES: O=C1CCC(N2Cc3cc(NC(=O)N4CCOc5ccccc54)ccc3C2=O)C(=O)N1] |
| 286 | 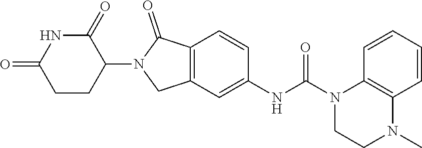 [SMILES: CN1CCN(C(=O)Nc2ccc3c(c2)CN(C2CCC(=O)NC2=O)C3=O)c2ccccc21] |
| 287 | 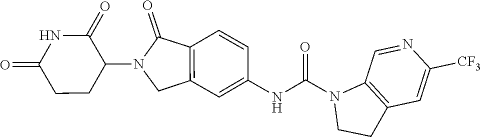 [SMILES: O=C1CCC(N2Cc3cc(NC(=O)N4CCc5cc(C(F)(F)F)ncc54)ccc3C2=O)C(=O)N1] |
| 288 | 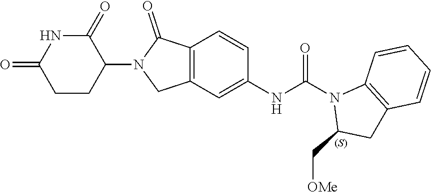 |
| 289 | 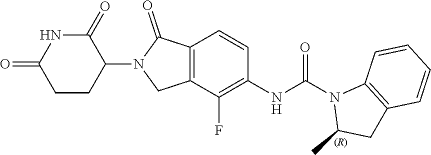 |
| 290 | 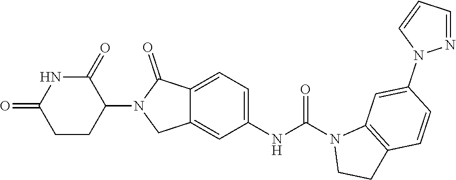 [SMILES: O=C1CCC(N2Cc3cc(NC(=O)N4CCc5ccc(-n6cccn6)cc54)ccc3C2=O)C(=O)N1] |
| 291 | 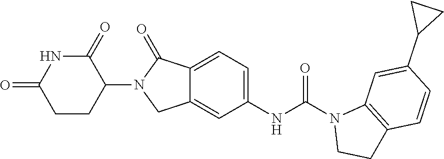 [SMILES: O=C1CCC(N2Cc3cc(NC(=O)N4CCc5ccc(C6CC6)cc54)ccc3C2=O)C(=O)N1] |
| 292 | 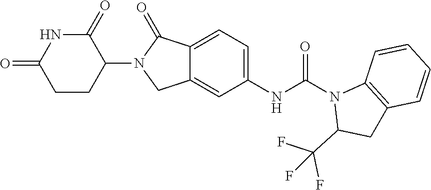 [SMILES: O=C1CCC(N2Cc3cc(NC(=O)N4c5ccccc5CC4C(F)(F)F)ccc3C2=O)C(=O)N1] |
| 293 | 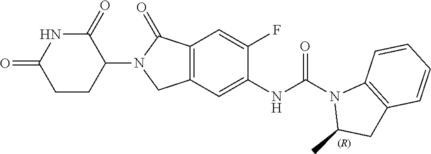 |
| 294 | 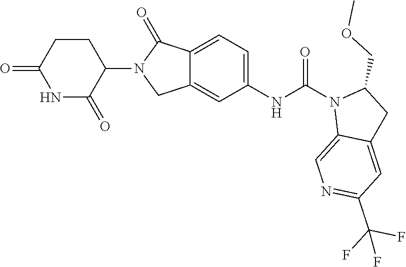 [SMILES: COC[C@@H]1Cc2cc(C(F)(F)F)ncc2N1C(=O)Nc1ccc2c(c1)CN(C1CCC(=O)NC1=O)C2=O] |
| 295 | 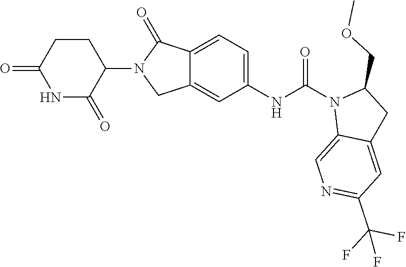 [SMILES: COC[C@H]1Cc2cc(C(F)(F)F)ncc2N1C(=O)Nc1ccc2c(c1)CN(C1CCC(=O)NC1=O)C2=O] |
| 296 | 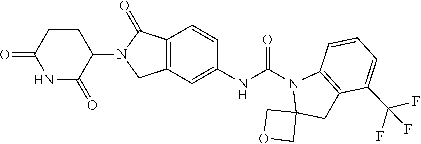 [SMILES: O=C1CCC(N2Cc3cc(NC(=O)N4c5cccc(C(F)(F)F)c5CC45COC5)ccc3C2=O)C(=O)N1] |
| 297 | 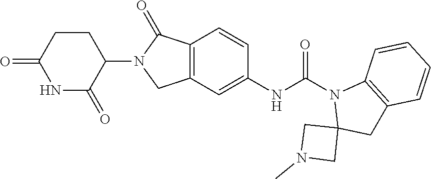 [SMILES: CN1CC2(Cc3ccccc3N2C(=O)Nc2ccc3c(c2)CN(C2CCC(=O)NC2=O)C3=O)C1] |
| 298 | 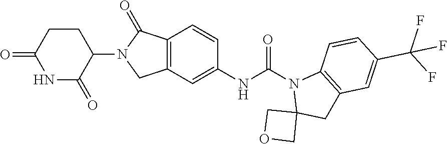 [SMILES: O=C1CCC(N2Cc3cc(NC(=O)N4c5ccc(C(F)(F)F)cc5CC45COC5)ccc3C2=O)C(=O)N1] |
| 299 | 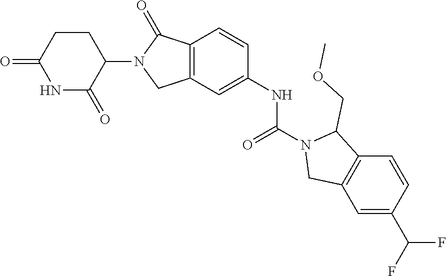 [SMILES: COCC1c2ccc(C(F)F)cc2CN1C(=O)Nc1ccc2c(c1)CN(C1CCC(=O)NC1=O)C2=O] |
| 300 | 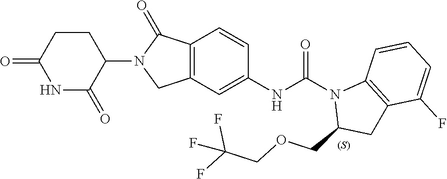 |
| 301 | 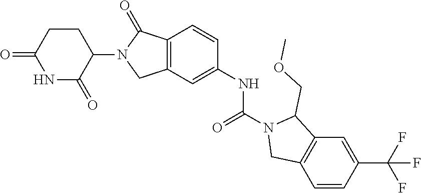 [SMILES: COCC1c2cc(C(F)(F)F)ccc2CN1C(=O)Nc1ccc2c(c1)CN(C1CCC(=O)NC1=O)C2=O] |
| 302 | 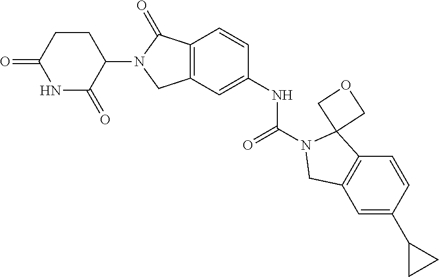 [SMILES: O=C1CCC(N2Cc3cc(NC(=O)N4Cc5cc(C6CC6)ccc5C45COC5)ccc3C2=O)C(=O)N1] |
| 303 | 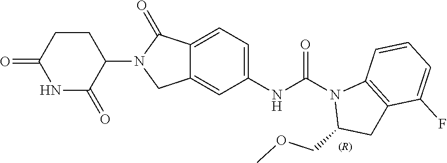 |
| 304 | 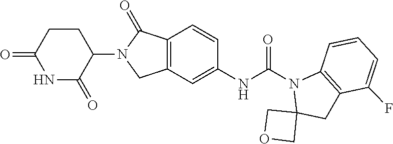 [SMILES: O=C1CCC(N2Cc3cc(NC(=O)N4c5cccc(F)c5CC45COC5)ccc3C2=O)C(=O)N1] |
| 305 | 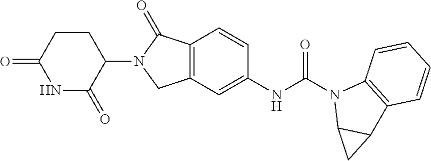 [SMILES: O=C1CCC(N2Cc3cc(NC(=O)N4c5ccccc5C5CC54)ccc3C2=O)C(=O)N1] |
| 306 | 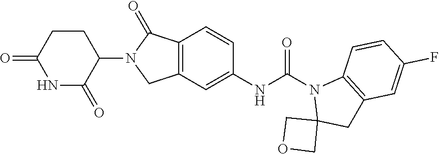 [SMILES: O=C1CCC(N2Cc3cc(NC(=O)N4c5ccc(F)cc5CC45COC5)ccc3C2=O)C(=O)N1] |
| 307 | 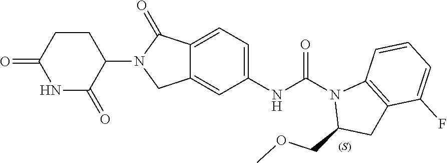 |
| 308 | 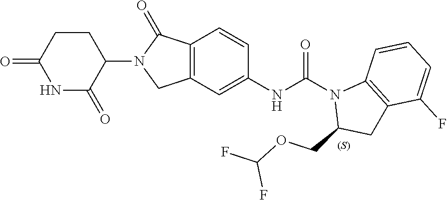 |
| 309 | 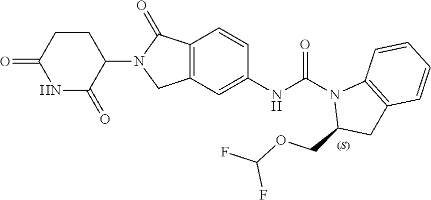 |
| 310 | 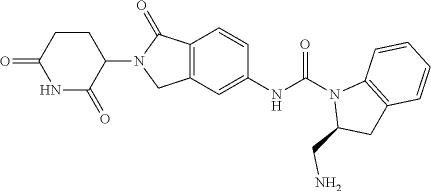 [SMILES: NC[C@@H]1Cc2ccccc2N1C(=O)Nc1ccc2c(c1)CN(C1CCC(=O)NC1=O)C2=O] |
| 311 | 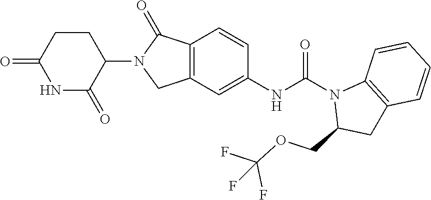 [SMILES: O=C1CCC(N2Cc3cc(NC(=O)N4c5ccccc5C[C@H]4COC(F)(F)F)ccc3C2=O)C(=O)N1] |
| 312 | 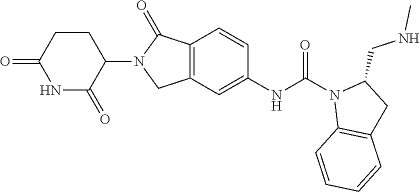 [SMILES: CNC[C@@H]1Cc2ccccc2N1C(=O)Nc1ccc2c(c1)CN(C1CCC(=O)NC1=O)C2=O] |
| 313 | 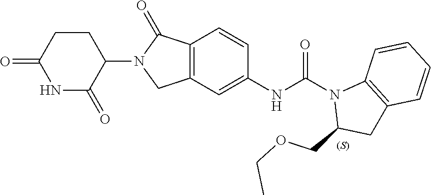 |
| 314 | 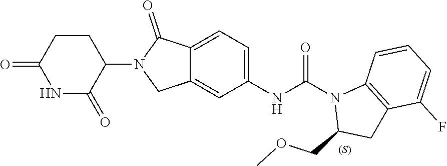 |
| 315 | 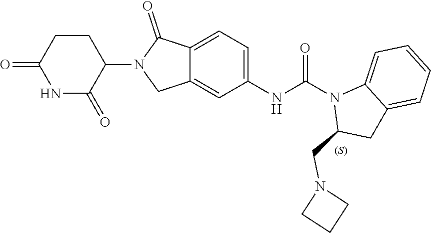 |
| 316 | 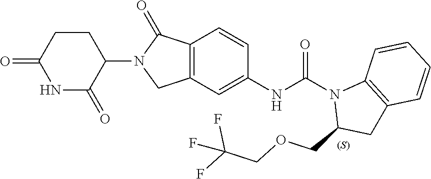 |
| 317 | 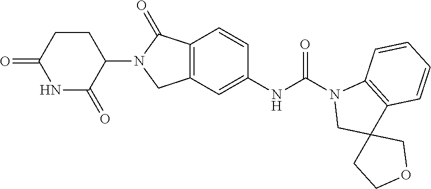 [SMILES: O=C1CCC(N2Cc3cc(NC(=O)N4CC5(CCOC5)c5ccccc54)ccc3C2=O)C(=O)N1] |
| 318 | 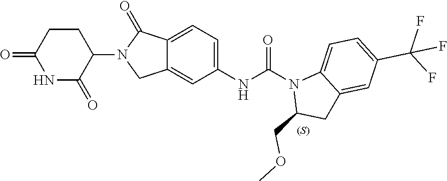 |
| 319 | 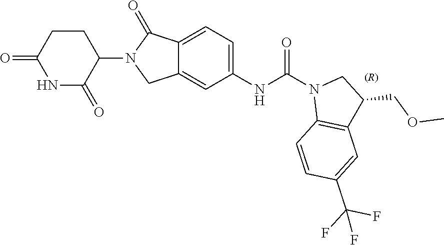 |
| 320 | 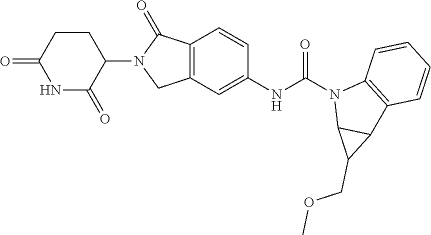 [SMILES: COCC1C2c3ccccc3N(C(=O)Nc3ccc4c(c3)CN(C3CCC(=O)NC3=O)C4=O)C12] |
| 321 | 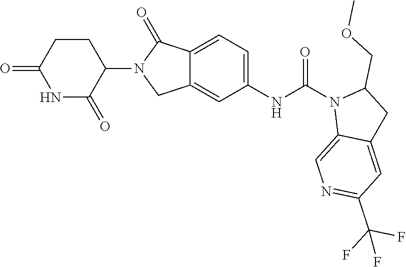 [SMILES: COCC1Cc2cc(C(F)(F)F)ncc2N1C(=O)Nc1ccc2c(c1)CN(C1CCC(=O)NC1=O)C2=O] |
| 322 | 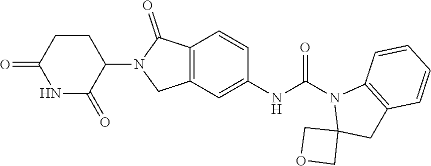 [SMILES: O=C1CCC(N2Cc3cc(NC(=O)N4c5ccccc5CC45COC5)ccc3C2=O)C(=O)N1] |
| 323 | 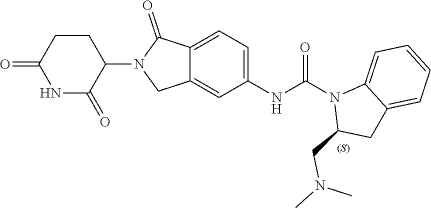 |
| 324 | 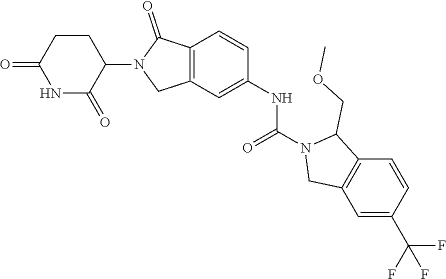 [SMILES: COCC1c2ccc(C(F)(F)F)cc2CN1C(=O)Nc1ccc2c(c1)CN(C1CCC(=O)NC1=O)C2=O] |
| 325 | 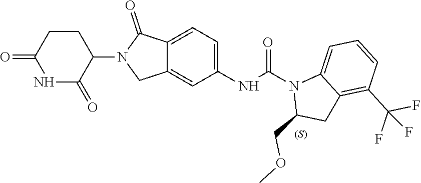 |
| 326 | 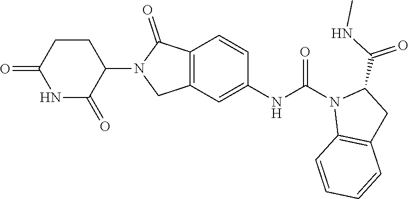 [SMILES: CNC(=O)[C@@H]1Cc2ccccc2N1C(=O)Nc1ccc2c(c1)CN(C1CCC(=O)NC1=O)C2=O] |
| 327 | 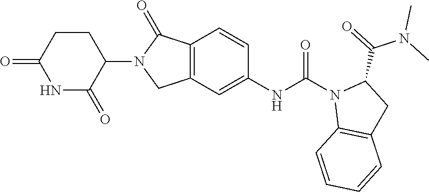 [SMILES: CN(C)C(=O)[C@@H]1Cc2ccccc2N1C(=O)Nc1ccc2c(c1)CN(C1CCC(=O)NC1=O)C2=O] |
| 328 | 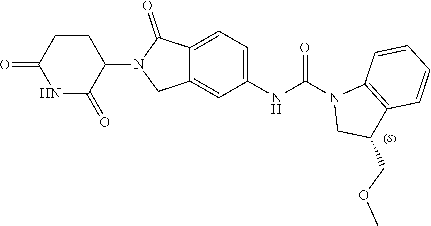 |
| 329 | 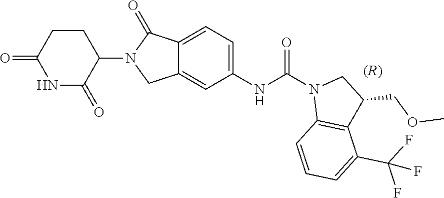 |
| 330 | 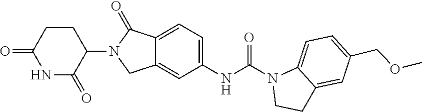 [SMILES: COCc1ccc2c(c1)CCN2C(=O)Nc1ccc2c(c1)CN(C1CCC(=O)NC1=O)C2=O] |
| 331 | 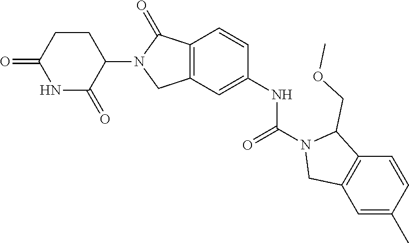 [SMILES: COCC1c2ccc(C)cc2CN1C(=O)Nc1ccc2c(c1)CN(C1CCC(=O)NC1=O)C2=O] |
| 332 | 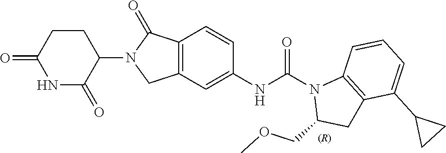 |
| 333 | 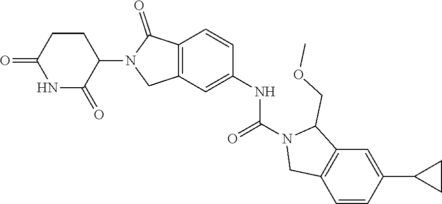 [SMILES: COCC1c2cc(C3CC3)ccc2CN1C(=O)Nc1ccc2c(c1)CN(C1CCC(=O)NC1=O)C2=O] |
| 334 | 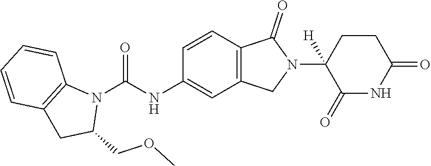 [SMILES: COC[C@@H]1Cc2ccccc2N1C(=O)Nc1ccc2c(c1)CN([C@H]1CCC(=O)NC1=O)C2=O] |
| 335 | 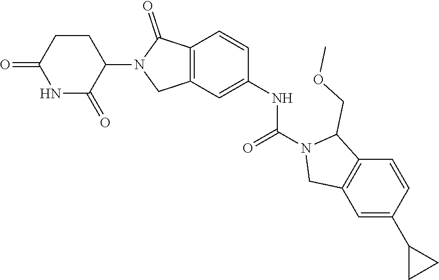 [SMILES: COCC1c2ccc(C3CC3)cc2CN1C(=O)Nc1ccc2c(c1)CN(C1CCC(=O)NC1=O)C2=O] |
| 336 | 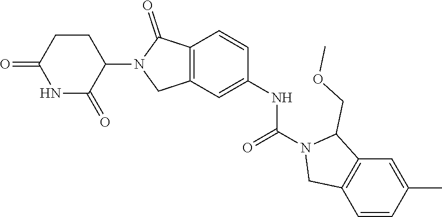 [SMILES: COCC1c2cc(C)ccc2CN1C(=O)Nc1ccc2c(c1)CN(C1CCC(=O)NC1=O)C2=O] |
| 337 | 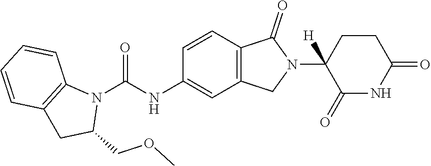 [SMILES: COC[C@@H]1Cc2ccccc2N1C(=O)Nc1ccc2c(c1)CN([C@@H]1CCC(=O)NC1=O)C2=O] |
| 338 | 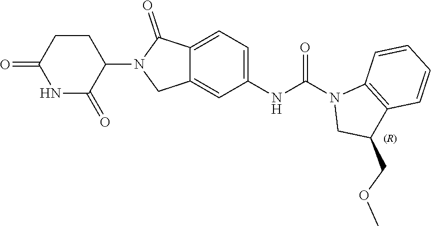 |
| 339 | 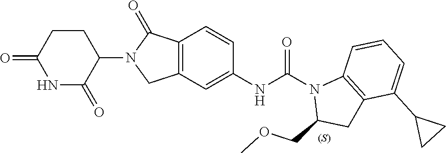 |
| 340 | 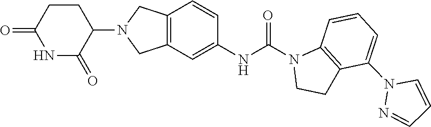 [SMILES: O=C1CCC(N2Cc3ccc(NC(=O)N4CCc5c4cccc5-n4cccn4)cc3C2)C(=O)N1] |
| 341 | 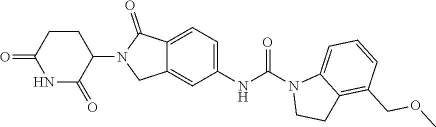 [SMILES: COCc1cccc2c1CCN2C(=O)Nc1ccc2c(c1)CN(C1CCC(=O)NC1=O)C2=O] |
| 342 | 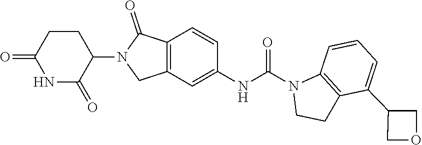 [SMILES: O=C1CCC(N2Cc3cc(NC(=O)N4CCc5c(C6COC6)cccc54)ccc3C2=O)C(=O)N1] |
| 343 | 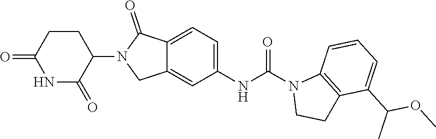 [SMILES: COC(C)c1cccc2c1CCN2C(=O)Nc1ccc2c(c1)CN(C1CCC(=O)NC1=O)C2=O] |
| 344 | 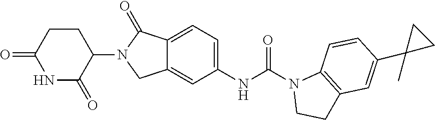 [SMILES: CC1(c2ccc3c(c2)CCN3C(=O)Nc2ccc3c(c2)CN(C2CCC(=O)NC2=O)C3=O)CC1] |
| 345 | 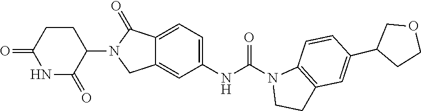 [SMILES: O=C1CCC(N2Cc3cc(NC(=O)N4CCc5cc(C6CCOC6)ccc54)ccc3C2=O)C(=O)N1] |
| 346 | 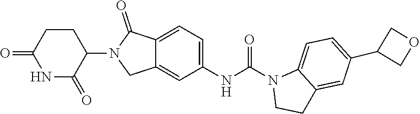 [SMILES: O=C1CCC(N2Cc3cc(NC(=O)N4CCc5cc(C6COC6)ccc54)ccc3C2=O)C(=O)N1] |
| 347 | 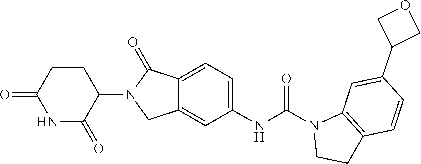 [SMILES: O=C1CCC(N2Cc3cc(NC(=O)N4CCc5ccc(C6COC6)cc54)ccc3C2=O)C(=O)N1] |
| 348 | 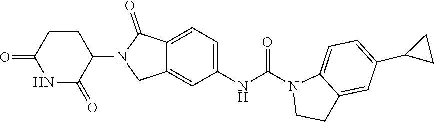 [SMILES: O=C1CCC(N2Cc3cc(NC(=O)N4CCc5cc(C6CC6)ccc54)ccc3C2=O)C(=O)N1] |
| 349 | 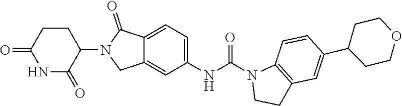 [SMILES: O=C1CCC(N2Cc3cc(NC(=O)N4CCc5cc(C6CCOCC6)ccc54)ccc3C2=O)C(=O)N1] |
| 350 | 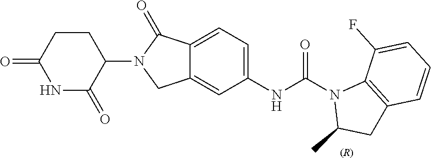 |
| 351 | 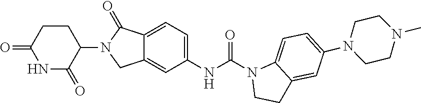 [SMILES: CN1CCN(c2ccc3c(c2)CCN3C(=O)Nc2ccc3c(c2)CN(C2CCC(=O)NC2=O)C3=O)CC1] |
| 352 | 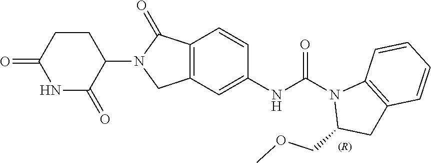 |
| 353 | 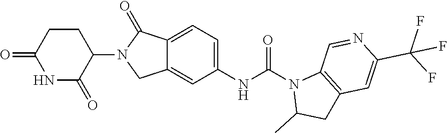 [SMILES: CC1Cc2cc(C(F)(F)F)ncc2N1C(=O)Nc1ccc2c(c1)CN(C1CCC(=O)NC1=O)C2=O] |
| 354 | 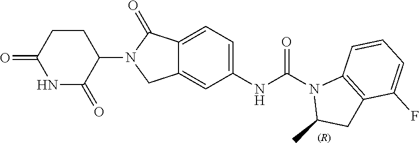 |
| 355 | 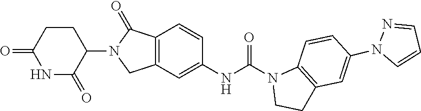 [SMILES: O=C1CCC(N2Cc3cc(NC(=O)N4CCc5cc(-n6cccn6)ccc54)ccc3C2=O)C(=O)N1] |
| 356 | 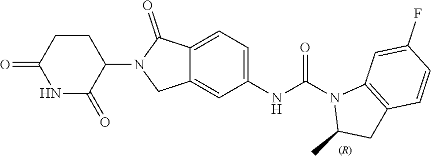 |
| 357 | 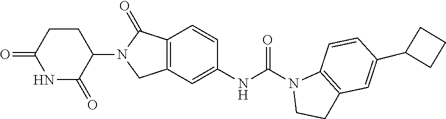 [SMILES: O=C1CCC(N2Cc3cc(NC(=O)N4CCc5cc(C6CCC6)ccc54)ccc3C2=O)C(=O)N1] |
| 358 | 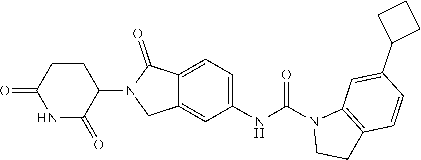 [SMILES: O=C1CCC(N2Cc3cc(NC(=O)N4CCc5ccc(C6CCC6)cc54)ccc3C2=O)C(=O)N1] |
| 359 | 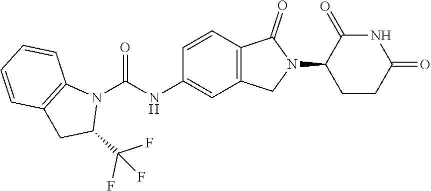 [SMILES: O=C1CC[C@@H](N2Cc3cc(NC(=O)N4c5ccccc5C[C@H]4C(F)(F)F)ccc3C2=O)C(=O)N1] |
| 360 | 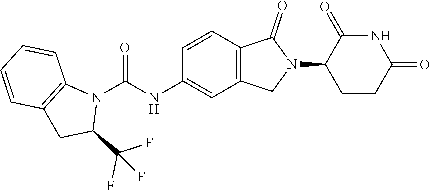 [SMILES: O=C1CC[C@@H](N2Cc3cc(NC(=O)N4c5ccccc5C[C@@H]4C(F)(F)F)ccc3C2=O)C(=O)N1] |
| 361 | 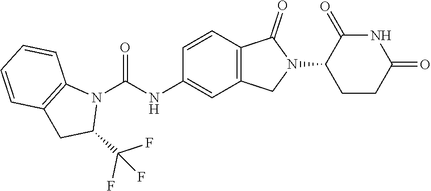 [SMILES: O=C1CC[C@H](N2Cc3cc(NC(=O)N4c5ccccc5C[C@H]4C(F)(F)F)ccc3C2=O)C(=O)N1] |
| 362 | 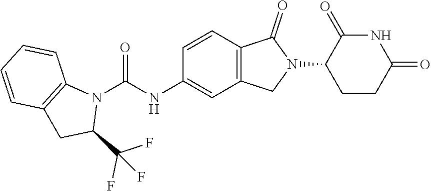 [SMILES: O=C1CC[C@H](N2Cc3cc(NC(=O)N4c5ccccc5C[C@@H]4C(F)(F)F)ccc3C2=O)C(=O)N1] |
| 363 | 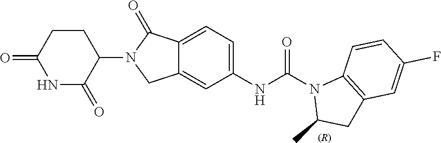 |
| 364 | 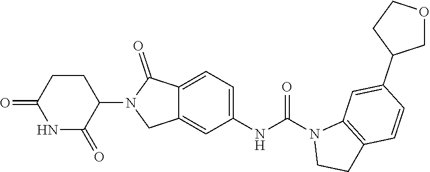 [SMILES: O=C1CCC(N2Cc3cc(NC(=O)N4CCc5ccc(C6CCOC6)cc54)ccc3C2=O)C(=O)N1] |
| 365 | 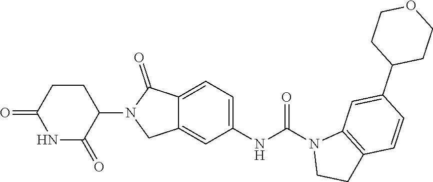 [SMILES: O=C1CCC(N2Cc3cc(NC(=O)N4CCc5ccc(C6CCOCC6)cc54)ccc3C2=O)C(=O)N1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to degrade casein kinase 1α (CK1 a) for the treatment of prevention of a disorder.

Casein kinase I (CK1) is a monomeric serine-threonine protein kinase with 7 isoforms: alpha, beta, gamma1, gamma2, gamma3, delta and epsilon. CK1 is involved in many cellular processes including DNA repair, cell division, nuclear localization and membrane transport. Isoforms are also integral to development. CK1α (casein kinase 1 alpha 1) is a protein coding gene that enables protein serine/threonine kinase activity involving in several processes, including negative regulation of canonical Wnt signaling pathway; peptidyl-serine phosphorylation; and positive regulation of proteasomal ubiquitin-dependent protein catabolic process. Through phosphorylation of different substrate proteins, CK1α is able to activate, stabilize, inactivate, or destabilize the functions of these substrate proteins, thus regulating their functions.

In one embodiment of the disclosure, a compound, or pharmaceutically acceptable salt thereof, or pharmaceutical composition described herein is administered to a subject to degrade CK1α in the subject.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading CK1α in a subject suffering from a disorder, comprising administering to the subject a therapeutically effective amount of a compound described herein, or pharmaceutically acceptable salt thereof, or pharmaceutical composition described herein. In some embodiments, the compound binds to cereblon and a CK1α protein to induce ubiquitination and subsequent proteasomal degradation of the CK1ca.

In certain embodiments, the compounds provided herein are degraders of a casein kinase 1. In certain embodiments, the compounds provided herein are degraders of casein kinase 1α (CK1α). In certain embodiments, the compounds provided herein are selective degraders of casein kinase 1α (CK1α). In certain embodiments, the compounds provided herein are degraders of human casein kinase 1α (CK1α). In certain embodiments, the compounds provided herein are selective degraders of human casein kinase 1α (CK1α).

Exemplary disorders that can be treated or prevented by the methods of the present disclosure include but are not limited to, cancer of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, non-Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading CK1α in a subject suffering from cancer (e.g., a cancer described herein), comprising administering to the subject a therapeutically effective amount of a compound described herein, or pharmaceutically acceptable salt thereof, or pharmaceutical composition described herein.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C). Additional examples of alkyl groups include n-heptyl (C₇), n-octyl (C₈) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like.

The term “cycloalkyl” as used herein refers to a radical of a saturated or partially unsaturated cyclic hydrocarbon group having from 3 to 12 ring carbon atoms (“C₃₋₁₂ cycloalkyl”) and zero heteroatoms in the ring system. In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 3 to 6 ring carbon atoms (“C₃₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C), cyclohexadienyl (C), and the like. Exemplary C₃_₈ cycloalkyl groups include, without limitation, the aforementioned C₃_₄ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “Spiro heterocyclyl,” “spiro heterocycle,” or “spirocyclic heterocycle,” refers to a polycyclic heterocyclyl with rings connected through one common atom (called a spiro atom), wherein the rings have one or more heteroatoms selected from the group consisting of N, O, and S(O)m (wherein m is an integer of 0 to 2) as ring atoms. Such spirocyclic heterocycles include bicyclic and tricyclic ring systems (e.g.,

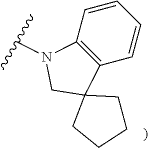 [SMILES: *N1CC2(CCCC2)c2ccccc21]

The term “bridged-heterocycle” as used herein refers to a 4, 5, 6, 7, 8, 9, 10, 11, or 12-membered heterocycle as defined herein connected at two non-adjacent atoms of the 4, 5, 6, 7 or 8-membered heterocycle with one or more (e.g., 1 or 2) 3, 4, 5 or 6-membered heterocycles or (C₃-C₇)carbocycles as defined herein. Such bridged-heterocycles include bicyclic and tricyclic ring systems (e.g., 6-azabicyclo[3.1.1]heptane, and

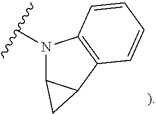 [SMILES: *N1c2ccccc2C2CC21]

As used herein, the term “haloalkyl” refers to alkyl group (as defined above) is substituted with one or more halogens. A monohaloalkyl radical, for example, may have a chlorine, bromine, iodine or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃) or trifluoroethoxy (—OCH₂CF₃).

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

The term “oxo” as used herein refers to ═O.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: ACN: acetonitrile; AIBN: azobisisobutyronitrile; BH₃·DMS: boron trifluoride methyl sulfide complex; BocNH₂: N-t-Butoxycarbonyl-amide; Boc₂O: di-tert-butyl dicarbonate; CDI: 1,1′-carbonyldiimidazole; CRBN: cereblon; DIEA: N,N-diisopropylethylamine; DMF: N,N-dimethylformamide; eq: equivalents; DMSO: dimethyl sulfoxide; EI: electron ionization; ESI: electrospray ionization; h: hours; HP-β-CD: hydroxypropyl-beta-cyclodextrin; HPLC: high-performance liquid chromatography; LCMS: liquid chromatography mass spectrometry; MeCN: acetonitrile; MS: mass spectrometry; MeI: methyl iodide; MTBE: tert-butyl methyl ether; NMR: nuclear magnetic resonance; Py: pyridine; TEA: triethylamine; and t-BuONa: sodium tert-butoxide.

### Example 1. Synthesis of Compound 111

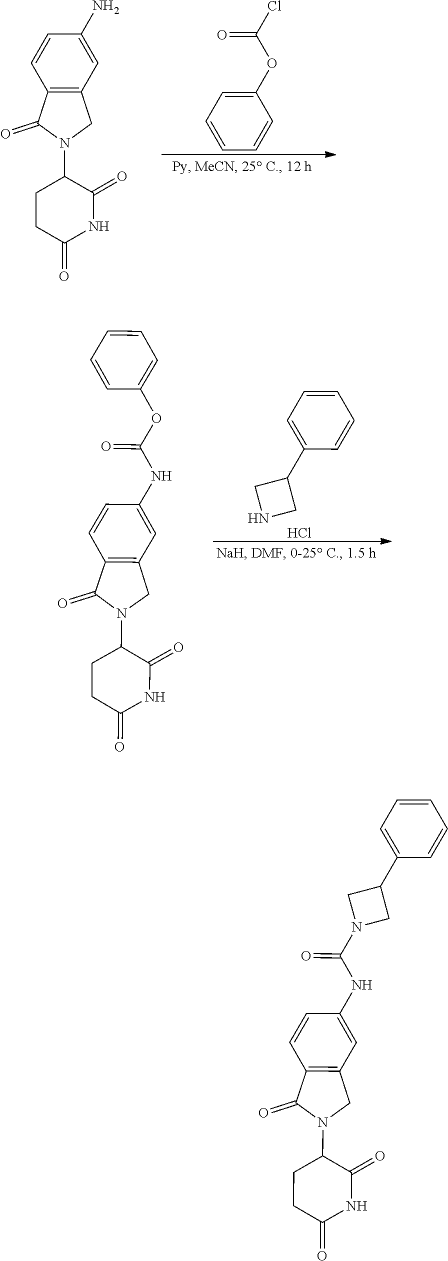 [SMILES: Nc1ccc2c(c1)CN(C1CCC(=O)NC1=O)C2=O ; O=C(Cl)Oc1ccccc1 ; O=C1CCC(N2Cc3cc(NC(=O)Oc4ccccc4)ccc3C2=O)C(=O)N1 ; c1ccc(C2CNC2)cc1 ; Cl ; O=C1CCC(N2Cc3cc(NC(=O)N4CC(c5ccccc5)C4)ccc3C2=O)C(=O)N1]

Step 1. To a solution of phenyl carbonochloridate (217 mg, 1.39 mmol, 1.20 eq) in dimethyl formamide (3.00 mL) was added pyridine (275 mg, 3.47 mmol, 3.00 eq) and 3-(5-amino-1-oxoisoindolin-2-yl)piperidine-2,6-dione (300 mg, 1.16 mmol, 1.00 eq) at 25° C. The resulting mixture was stirred at 25° C. for 12 h. The reaction mixture was quenched with water (20 mL). The resulting mixture was extracted with ethyl acetate (3×30 mL). The organic layers were collected. The organic layers were washed with brine (3×10 mL), dried over sodium sulfate, filtered and concentrated under reduced pressure to afford phenyl (2-(2,6-dioxopiperidin-3-yl)-1-oxoisoindolin-5-yl)carbamate (350 mg, crude) as light yellow oil.

¹H NMR (400 MHz, DMSO-d₆) δ 10.98 (s, 1H), 10.64 (s, 1H), 7.82 (s, 1H), 7.69 (d, J=8.4 Hz, 1H), 7.58 (dd, J=1.3, 8.3 Hz, 1H), 7.51-7.43 (m, 4H), 7.39 (s, 1H), 5.09 (dd, J=5.1, 13.3 Hz, 1H), 4.51-4.23 (m, 2H), 2.59 (br d, J=16.9 Hz, 1H), 2.54-2.52 (m, 1H), 2.42-2.34 (m, 1H), 2.05-1.92 (m, 1H).

Step 2. To a solution of 3-phenylazetidine hydrochloride (50.0 mg, 295 umol, 1.00 eq) in dimethyl formamide (5.00 mL) was added sodium hydride (35.4 mg, 884 umol, 60% purity, 3.00 eq) at 0° C. The mixture was stirred at 25° C. for 0.5 h, then phenyl (2-(2,6-dioxopiperidin-3-yl)-1-oxoisoindolin-5-yl)carbamate (224 mg, 589 umol, 2.00 eq) was added and the resulting reaction mixture was stirred at 25° C. for 1 h. The reaction mixture was quenched with formic acid to adjust pH=5. The crude product was purified by Prep-HPLC (column: Phenomenex luna C18 150*25 mm*10 um; mobile phase: [water (FA)-ACN]; B %: 23%-53%, 9 min). The desired fraction was collected and lyophilized to afford N-(2-(2,6-dioxopiperidin-3-yl)-1-oxoisoindolin-5-yl)-3-phenylazetidine-1-carboxamide (35.0 mg, 82.8 umol, 28% yield, 99% purity) as a white solid.

¹H NMR (400 MHz, DMSO-d₆) δ=10.98 (s, 1H), 8.87 (s, 1H), 7.86 (s, 1H), 7.59 (s, 2H), 7.42-7.34 (m, 4H), 7.30-7.23 (m, 1H), 5.07 (dd, J=5.1, 13.1 Hz, 1H), 4.46-4.36 (m, 3H), 4.29-4.22 (m, 1H), 3.98 (dd, J=6.2, 8.1 Hz, 2H), 3.90-3.78 (m, 1H), 2.98-2.84 (m, 1H), 2.61 (br s, 1H), 2.42-2.34 (m, 1H), 2.02-1.94 (m, 1H).

### Example 2. Synthesis of Compound 106

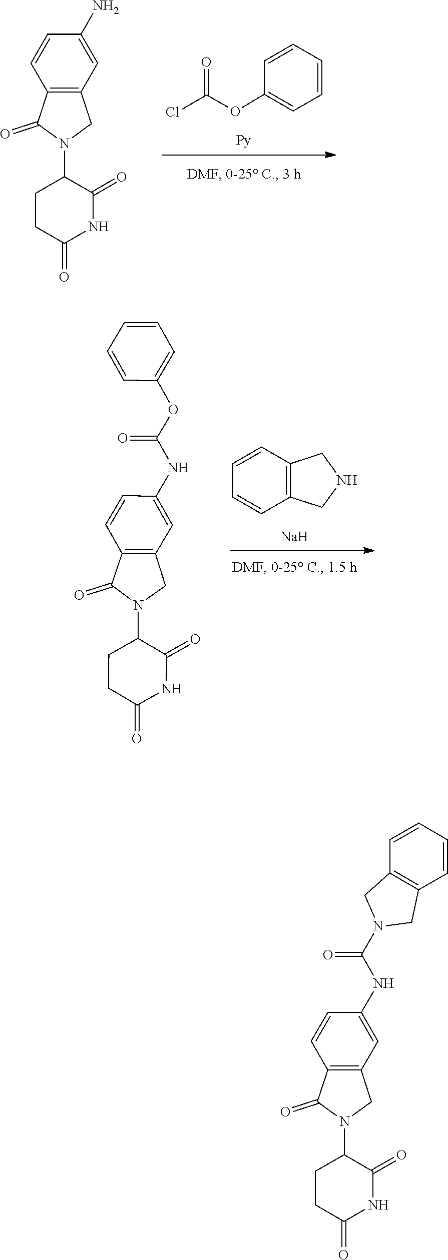

Step 1. To a solution of 3-(5-amino-1-oxoisoindolin-2-yl)piperidine-2,6-dione (1.00 g, 3.86 mmol, 1.00 eq) in N,N-dimethyl formamide (10.0 mL) was added pyridine (915 mg, 11.6 mmol, 934 μL, 3.00 eq) and phenyl carbonochloridate (625 mg, 3.99 mmol, 500 uL, 1.03 eq) at 0° C. The resulting mixture was stirred at 25° C. for 3 h. The mixture was diluted with water (100 mL) and extracted with ethyl acetate (3×100 mL). The organic layers were collected, dried over anhydrous sodium sulfate and concentrated in vacuum to give crude product, which was purified by column chromatography (SiO₂, petroleum ether/ethyl acetate=50/1 to 1/1) to afford phenyl (2-(2,6-dioxopiperidin-3-yl)-1-oxoisoindolin-5-yl)carbamate (400 mg, 1.05 mmol, 27% yield) as a gray solid.

¹H NMR (400 MHz, DMSO-d₆) δ=10.98 (s, 1H), 10.64 (s, 1H), 7.81 (s, 1H), 7.69 (d, J=8.4 Hz, 1H), 7.58 (dd, J=1.1, 8.4 Hz, 1H), 7.48-7.42 (m, 2H), 7.31-7.27 (m, 1H), 7.27 (s, 1H), 7.25 (s, 1H), 5.09 (dd, J=5.1, 13.3 Hz, 1H), 4.48-4.40 (m, 1H), 4.35-4.24 (m, 1H), 2.96-2.86 (m, 1H), 2.59 (br d, J=17.5 Hz, 1H), 2.37 (br dd, J=4.6, 13.2 Hz, 1H), 2.05-1.99 (m, 1H).

Step 2. To a solution of isoindoline (100 mg, 839 umol, 95.2 uL, 1.00 eq) in N,N-dimethyl formamide (10.0 mL) was added sodium hydride (101 mg, 2.52 mmol, 60% purity, 3.00 eq) at 0° C. The mixture was stirred at 25° C. for 0.5 h, then phenyl (2-(2,6-dioxopiperidin-3-yl)-1-oxoisoindolin-5-yl)carbamate (318 mg, 839 umol, 1.00 eq) was added and the resulting reaction mixture was stirred at 25° C. for 1 h. The pH of reaction mixture was adjusted to 6 with formic acid and filtered. The filter cake was washed with water (5 mL), dried in vacuum to afford N-(2-(2,6-dioxopiperidin-3-yl)-1-oxoisoindolin-5-yl)isoindoline-2-carboxamide (115.7 mg, 281 umol, 33% yield, 97% purity) as a grey solid.

¹H NMR (400 MHz, DMSO-d₆) δ=10.97 (s, 1H), 8.71 (s, 1H), 7.93 (s, 1H), 7.70-7.64 (m, 1H), 7.63-7.55 (m, 1H), 7.42-7.35 (m, 2H), 7.35-7.29 (m, 2H), 5.08 (dd, J=5.1, 13.2 Hz, 1H), 4.81 (s, 4H), 4.45-4.36 (m, 1H), 4.32-4.24 (m, 1H), 2.98-2.83 (m, 1H), 2.60 (br d, J=17.1 Hz, 1H), 2.44-2.35 (m, 1H), 2.04-1.95 (m, 1H).

### Example 3. Synthesis of Compound 103

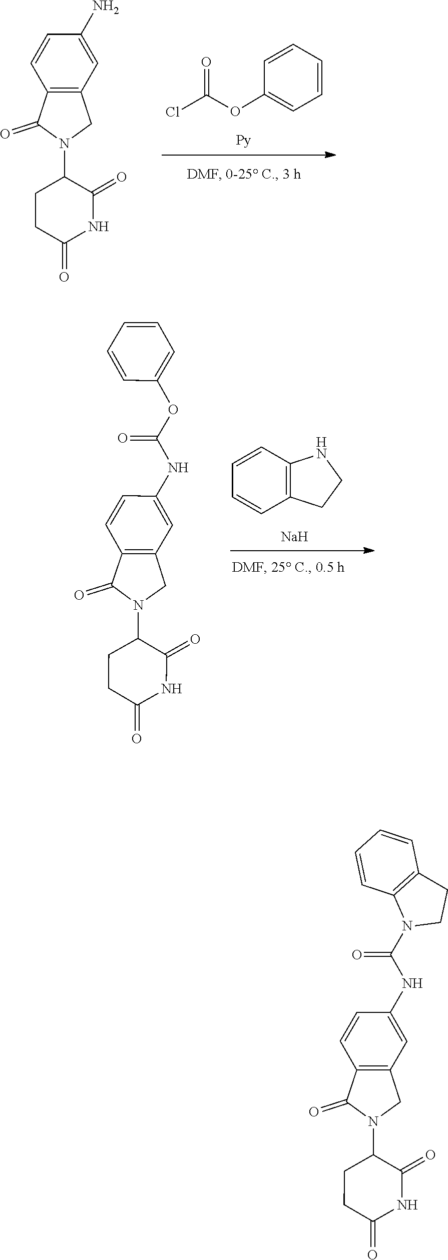

Step 1. To a solution of 3-(5-amino-1-oxo-isoindolin-2-yl)piperidine-2,6-dione (1.00 g, 3.86 mmol, 1.00 eq) in dimethyl formamide (5.00 mL) was added phenyl carbonochloridate (634 mg, 4.05 mmol, 507 uL, 1.05 eq) and pyridine (915 mg, 11.5 mmol, 934 uL, 3.00 eq). The mixture was stirred at 25° C. for 3 h. The reaction mixture was concentrated under reduced pressure to give a residue. The crude product was purified by reversed-phase HPLC (column: spherical C18, 20-45 um, 100 Å, SW 120, mobile phase: [water (0.1% Formic Acid)-ACN]). The desired fraction was collected and lyophilized to afford phenyl (2-(2,6-dioxopiperidin-3-yl)-1-oxoisoindolin-5-yl)carbamate (120 mg, 316 umol, 8% yield) as a white solid.

¹H NMR (400 MHz, DMSO-d₆) δ=10.98 (s, 1H), 10.63 (s, 1H), 7.81 (s, 1H), 7.69 (d, J=8.4 Hz, 1H), 7.58 (br d, J=7.6 Hz, 1H), 7.47-7.42 (m, 2H), 7.28 (br s, 1H), 7.27-7.24 (m, 2H), 5.09 (dd, J=5.1, 13.4 Hz, 1H), 4.48-4.41 (m, 1H), 4.34-4.26 (m, 1H), 2.96-2.85 (m, 1H), 2.59 (br d, J=19.4 Hz, 1H), 2.37 (br dd, J=4.3, 12.9 Hz, 1H), 2.05-1.97 (m, 1H).

Step 2. To a solution of phenyl (2-(2,6-dioxopiperidin-3-yl)-1-oxoisoindolin-5-yl)carbamate (100 mg, 263 umol, 1.00 eq) in dimethyl formamide (1.00 mL) was added indoline (37.6 mg, 316 umol, 35.5 uL, 1.20 eq) and sodium hydride (21.0 mg, 527 umol, 60% purity, 2.00 eq). The mixture was stirred at 25° C. for 0.5 h. The reaction mixture was quenched with formic acid (0.100 mL). The solution was purified by prep-HPLC (column: mobile phase: [column: mobile phase: [water (FA)-ACN]; B %: 22%-52%, 12 min) and lyophilized to give N-(2-(2,6-dioxopiperidin-3-yl)-1-oxoisoindolin-5-yl)indoline-1-carboxamide (21.65 mg, 53.5 umol, 20% yield) as a grey solid.

¹H NMR (400 MHz, DMSO-d₆) δ=10.99 (s, 1H), 8.85 (s, 1H), 7.93 (s, 1H), 7.89 (d, J=8.0 Hz, 1H), 7.66 (s, 2H), 7.22 (d, J=7.3 Hz, 1H), 7.14 (t, J=7.7 Hz, 1H), 6.95-6.91 (m, 1H), 5.10 (dd, J=5.1, 13.3 Hz, 1H), 4.47-4.42 (m, 1H), 4.33-4.27 (m, 1H), 4.18 (t, J=8.6 Hz, 2H), 3.20 (t, J=8.6 Hz, 2H), 2.97-2.88 (m, 1H), 2.62 (br d, J=2.5 Hz, 1H), 2.39 (br dd, J=4.4, 13.2 Hz, 1H), 2.03-1.97 (m, 1H).

### Example 4. Synthesis of Compound 104

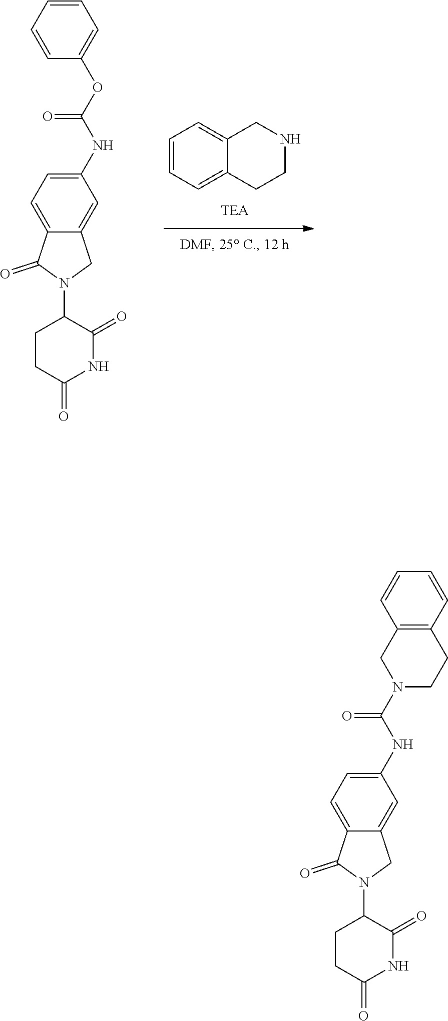 [SMILES: O=C1CCC(N2Cc3cc(NC(=O)Oc4ccccc4)ccc3C2=O)C(=O)N1 ; c1ccc2c(c1)CCNC2 ; O=C1CCC(N2Cc3cc(NC(=O)N4CCc5ccccc5C4)ccc3C2=O)C(=O)N1]

To a solution of phenyl (2-(2,6-dioxopiperidin-3-yl)-1-oxoisoindolin-5-yl)carbamate (100 mg, 263 umol, 1.00 eq) and 1,2,3,4-tetrahydroisoquinoline (35.1 mg, 263 umol, 33.1 uL, 1.00 eq) in N,N-dimethyl formamide (1.00 mL) was added triethylamine (80.0 mg, 790 umol, 110 μL, 3.00 eq). The mixture was stirred at 25° C. for 12 h. The mixture was filtered to give the filtrate, which was purified by Prep-HPLC (column: Phenomenex luna C18 150*25 mm*10 um; mobile phase: [water (FA)-ACN]; B %: 20%-50%, 15 min). The desired fraction was collected and lyophilized to afford N-(2-(2,6-dioxopiperidin-3-yl)-1-oxoisoindolin-5-yl)-3,4-dihydroisoquinoline-2(1H)-carboxamide (15.0 mg, 35.4 umol, 13% yield, 99% purity) as a white solid.

¹H NMR (400 MHz, DMSO-d₆) δ=10.96 (br s, 1H), 8.94 (s, 1H), 7.81 (s, 1H), 7.58 (br d, J=3.5 Hz, 2H), 7.19 (s, 4H), 5.07 (br dd, J=4.8, 13.3 Hz, 1H), 4.67 (s, 2H), 4.44-4.34 (m, 1H), 4.31-4.20 (m, 1H), 3.73 (br t, J=5.6 Hz, 2H), 2.97-2.88 (m, 1H), 2.87 (br t, J=5.3 Hz, 2H), 2.59 (br d, J=18.0 Hz, 1H), 2.44-2.34 (m, 1H), 2.04-1.91 (m, 1H).

### Example 5. Synthesis of Compound 107

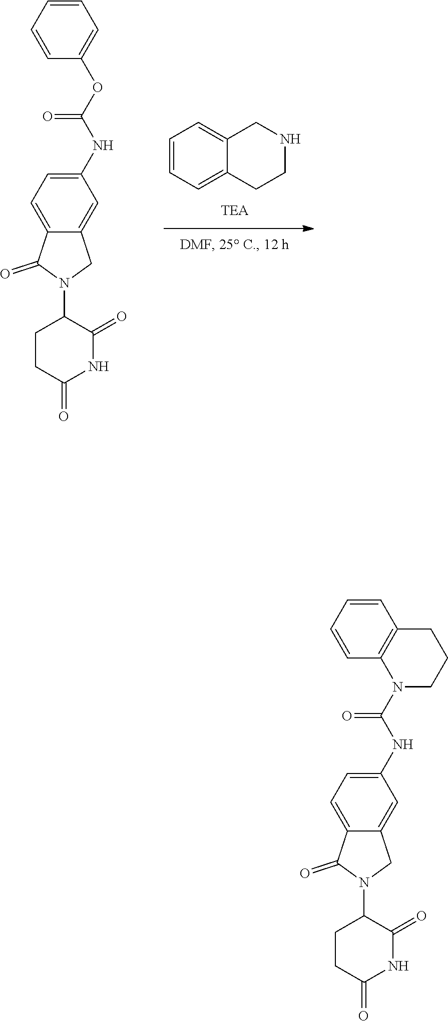 [SMILES: O=C1CCC(N2Cc3cc(NC(=O)Oc4ccccc4)ccc3C2=O)C(=O)N1 ; c1ccc2c(c1)CCNC2 ; O=C1CCC(N2Cc3cc(NC(=O)N4CCCc5ccccc54)ccc3C2=O)C(=O)N1]

To a solution of phenyl (2-(2,6-dioxopiperidin-3-yl)-1-oxoisoindolin-5-yl)carbamate (100 mg, 264 umol, 1.00 eq) and 1,2,3,4-tetrahydroquinoline (35.1 mg, 264 umol, 1.00 eq) in N,N-dimethyl formamide (2.00 mL) was added triethylamine (80.0 mg, 791 umol, 110 μL, 3.00 eq). The mixture was stirred at 25° C. for 12 h. The mixture was filtered and the filtrate was purified by Prep-HPLC (column: Phenomenex Luna C18 100*30 mm*5 um; mobile phase: [water (FA)-ACN]; B %: 20%-50%, 8 min). The desired fraction was collected and lyophilized to afford N-(2-(2,6-dioxopiperidin-3-yl)-1-oxoisoindolin-5-yl)-3,4-dihydroquinoline-1(2H)-carboxamide (34.0 mg, 81.3 umol, 30% yield, 98% purity) as a white solid.

¹H NMR (400 MHz, DMSO-d₆) δ=10.96 (s, 1H), 9.23 (s, 1H), 7.79 (d, J=1.1 Hz, 1H), 7.63-7.59 (m, 1H), 7.56-7.52 (m, 1H), 7.34 (d, J=7.4 Hz, 1H), 7.18-7.09 (m, 2H), 7.02-6.96 (m, 1H), 5.08 (dd, J=5.1, 13.2 Hz, 1H), 4.45-4.36 (m, 1H), 4.30-4.23 (m, 1H), 3.76-3.70 (m, 2H), 2.93-2.86 (m, 1H), 2.76 (t, J=6.6 Hz, 2H), 2.69-2.65 (m, 1H), 2.38 (br dd, J=4.4, 13.1 Hz, 1H), 2.03-2.02 (m, 1H), 1.91 (quin, J=6.4 Hz, 2H)

### Example 6. Synthesis of Compound 102

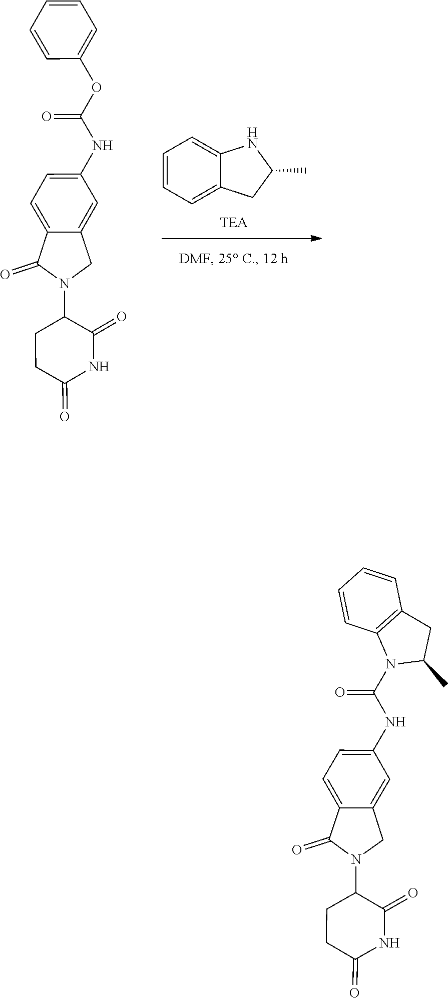

To a solution of phenyl (2-(2,6-dioxopiperidin-3-yl)-1-oxoisoindolin-5-yl)carbamate (56.9 mg, 150 umol, 1.00 eq) and (R)-2-methylindoline (20.0 mg, 150 umol, 1.00 eq) in N,N-dimethyl formamide (2.00 mL) was added triethylamine (45.6 mg, 450 umol, 62.7 uL, 3.00 eq). The mixture was stirred at 25° C. for 12 h. The mixture was filtered to give the filtrate, which was purified by prep-HPLC (column: Phenomenex luna C18 150*25 mm*10 um; mobile phase: [water (FA)-ACN]; B %: 50%-80%, 15 min). The desired fraction was collected and the aqueous solution was lyophilized to afford (2R)—N-(2-(2,6-dioxopiperidin-3-yl)-1-oxoisoindolin-5-yl)-2-methylindoline-1-carboxamide (9.11 mg, 21.1 umol, 14% yield, 97% purity) as a yellow solid.

¹H NMR (400 MHz, DMSO-d₆) δ=10.98 (s, 1H), 8.96 (s, 1H), 7.93-7.83 (m, 2H), 7.65 (s, 2H), 7.24 (d, J=7.5 Hz, 1H), 7.15 (t, J=7.7 Hz, 1H), 6.98-6.91 (m, 1H), 5.09 (dd, J=4.9, 13.3 Hz, 1H), 4.94-4.85 (m, 1H), 4.47-4.39 (m, 1H), 4.34-4.26 (m, 1H), 3.40 (dd, J=9.2, 15.9 Hz, 1H), 2.97-2.85 (m, 1H), 2.63-2.57 (m, 1H), 2.52 (br s, 1H), 2.44-2.36 (m, 1H), 2.05-1.94 (m, 1H), 1.24 (d, J=6.1 Hz, 3H).

### Example 7. Synthesis of Compound 101

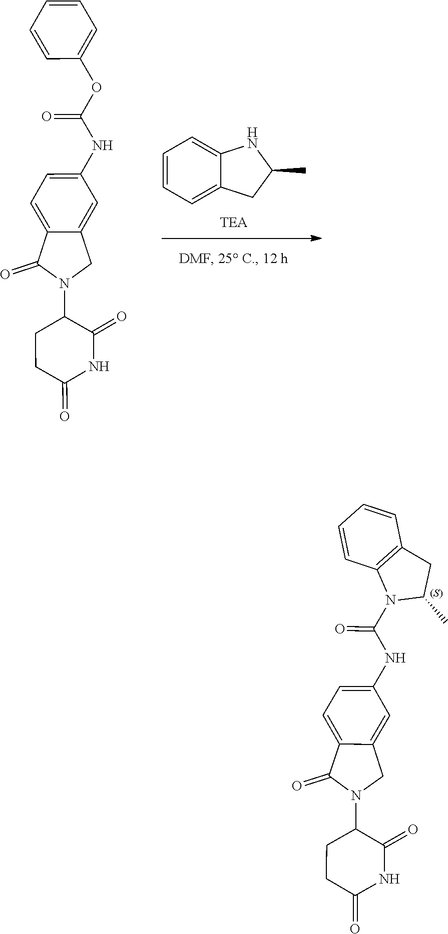

To a solution of phenyl (2-(2,6-dioxopiperidin-3-yl)-1-oxoisoindolin-5-yl)carbamate (143 mg, 377 umol, 1.00 eq) in N,N-dimethyl formamide (2.50 mL) was added (S)-2-methylindoline (50.0 mg, 375 umol, 1.00 eq). Then triethylamine (114 mg, 1.13 mmol, 157 μL, 3.00 eq) was added into the mixture and the mixture was stirred at 25° C. for 12 h. The mixture was filtered. The filtrate was purified by Prep-HPLC (column: Phenomenex luna C18 150*25 mm*10 um; mobile phase: [water (FA)-ACN]; B %: 29%-59%, 10 min). The desired fraction was collected and lyophilized to afford (2S)—N-(2-(2,6-dioxopiperidin-3-yl)-1-oxoisoindolin-5-yl)-2-methylindoline-1-carboxamide (6.23 mg, 14.6 umol, 3% yield, 98% purity) as a white solid.

¹H NMR (400 MHz, DMSO-d₆) δ=10.96 (s, 1H), 8.95 (s, 1H), 8.04-7.76 (m, 2H), 7.65 (s, 2H), 7.24 (d, J=7.3 Hz, 1H), 7.15 (t, J=7.8 Hz, 1H), 6.98-6.91 (m, 1H), 5.09 (dd, J=5.1, 13.3 Hz, 1H), 4.95-4.84 (m, 1H), 4.48-4.38 (m, 1H), 4.35-4.25 (m, 1H), 3.40 (dd, J=9.1, 16.1 Hz, 1H), 2.98-2.85 (m, 1H), 2.71 (br d, J=15.9 Hz, 1H), 2.63-2.57 (m, 1H), 2.39 (br dd, J=4.6, 13.3 Hz, 1H), 2.06-1.94 (m, 1H), 1.24 (d, J=6.3 Hz, 3H).

### Example 8. Synthesis of Compound 283 and Compound 284

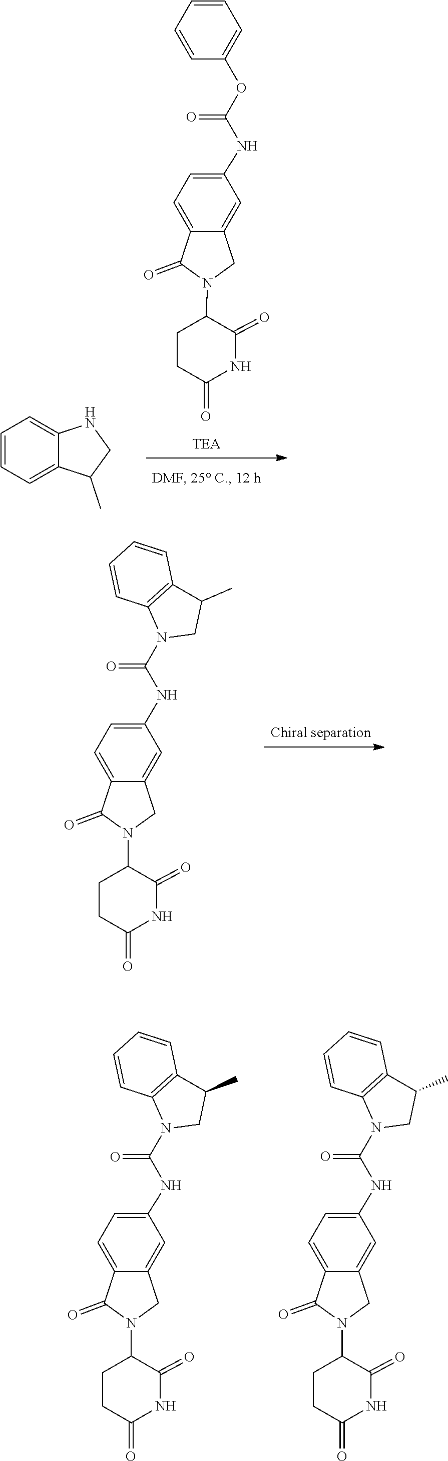

Step 1. To a solution of phenyl (2-(2,6-dioxopiperidin-3-yl)-1-oxoisoindolin-5-yl)carbamate (2.56 g, 6.76 mmol, 1.00 eq) in N,N-dimethyl formamide (10.0 mL) was added 3-methylindoline (900 mg, 6.76 mmol, 1.00 eq) and triethylamine (2.05 g, 20.2 mmol, 2.82 mL, 3.00 eq). The mixture was stirred at 25° C. for 12 h. Then phenyl (2-(2,6-dioxopiperidin-3-yl)-1-oxoisoindolin-5-yl)carbamate (1.30 g, 3.43 mmol, 0.500 eq) was added into the mixture. The mixture was filtered and the filter cake was triturated with N,N-dimethyl formamide (3 mL) at 25° C. for 30 min. The mixture was filtered to afford N-(2-(2,6-dioxopiperidin-3-yl)-1-oxoisoindolin-5-yl)-3-methylindoline-1-carboxamide (3.60 g, crude) as a yellow solid.

¹H NMR (400 MHz, DMSO-d₆) δ=10.98 (s, 1H), 9.06 (s, 1H), 7.95 (s, 1H), 7.86 (d, J=8.0 Hz, 1H), 7.73 (d, J=8.4 Hz, 1H), 7.62 (d, J=8.3 Hz, 1H), 7.22 (d, J=7.4 Hz, 1H), 7.14 (t, J=7.7 Hz, 1H), 6.98-6.91 (m, 1H), 5.08 (dd, J=5.0, 13.3 Hz, 1H), 4.46-4.38 (m, 2H), 4.32-4.25 (m, 1H), 3.76 (dd, J=7.0, 10.0 Hz, 1H), 3.57-3.45 (m, 1H), 2.97-2.89 (m, 1H), 2.59 (br d, J=17.3 Hz, 1H), 2.39 (br dd, J=4.3, 13.1 Hz, 1H), 2.04-1.95 (m, 1H), 1.31 (d, J=6.9 Hz, 3H).

Step 2. The racemic material of N-(2-(2,6-dioxopiperidin-3-yl)-1-oxoisoindolin-5-yl)-3-methylindoline-1-carboxamide (500 mg, crude) was purified by SFC (column: DAICEL CHIRALPAK AS (250 mm*50 mm, 10 um); mobile phase: [IPA-ACN]; B %: 50%-50%, A13; 598 min). The fractions were concentrated under reduced pressure to give two parts. Part 1 was separated by Chiral SFC column: DAICEL CHIRALCEL OD (250 mm*50 mm, 10 um); mobile phase: [IPA-ACN]; B %: 57%-57%, A13.4; 110 min. Part 2 was separated by Chiral SFC column: DAICEL CHIRALCEL OD (250 mm*50 mm, 10 um); mobile phase: [IPA-ACN]; B %: 57%-57%, A8; 200 min. Then the fractions were concentrated under reduced pressure to give four peaks.

Peak 1 was purified by Prep-HPLC (column: Phenomenex luna C18 150*25 mm*10 um; mobile phase: [water (FA)-ACN]; B %: 27%-57%, 10 min). The desired fraction was collected and lyophilized to afford (3R)—N-(2-(2,6-dioxopiperidin-3-yl)-1-oxoisoindolin-5-yl)-3-methylindoline-1-carboxamide. (12.33 mg, 29.47 umol, 12% yield) as a white solid.

¹H NMR (400 MHz, DMSO-d₆) δ=10.95 (br s, 1H), 8.84 (s, 1H), 7.92 (s, 1H), 7.87 (d, J=8.0 Hz, 1H), 7.68-7.62 (m, 2H), 7.23 (d, J=7.4 Hz, 1H), 7.16 (t, J=7.8 Hz, 1H), 6.96 (t, J=7.4 Hz, 1H), 5.09 (dd, J=5.1, 13.3 Hz, 1H), 4.46-4.40 (m, 1H), 4.39-4.27 (m, 2H), 3.72 (dd, J=6.9, 10.1 Hz, 1H), 3.57-3.49 (m, 1H), 2.96-2.87 (m, 1H), 2.60 (br dd, J=2.3, 15.1 Hz, 1H), 2.39 (br dd, J=4.4, 13.1 Hz, 1H), 2.04-1.96 (m, 1H), 1.32 (d, J=6.9 Hz, 3H) MS (ESI) m/z 419.0 [M+H]+ SFC, retention time: 1.202; 1.508 min

Peak 2 was purified by Prep-HPLC (column: Phenomenex luna C18 150*25 mm*10 um; mobile phase: [water (FA)-ACN]; B %: 30%-60%, 10 min). The desired fraction was collected and lyophilized to afford (3R)—N-(2-(2,6-dioxopiperidin-3-yl)-1-oxoisoindolin-5-yl)-3-methylindoline-1-carboxamide. (19.08 mg, 45.60 umol, 16% yield) as a white solid.

¹H NMR (400 MHz, DMSO-d₆) δ=11.13-10.75 (m, 1H), 8.85 (s, 1H), 7.92 (s, 1H), 7.87 (d, J=8.1 Hz, 1H), 7.69-7.60 (m, 2H), 7.24 (d, J=7.3 Hz, 1H), 7.15 (t, J=7.8 Hz, 1H), 6.99-6.92 (m, 1H), 5.09 (dd, J=5.1, 13.3 Hz, 1H), 4.46-4.40 (m, 1H), 4.39-4.26 (m, 2H), 3.71 (dd, J=7.0, 9.9 Hz, 1H), 3.57-3.48 (m, 1H), 2.98-2.85 (m, 1H), 2.60 (br dd, J=2.3, 15.4 Hz, 1H), 2.44-2.35 (m, 1H), 2.05-1.94 (m, 1H), 1.32 (d, J=6.9 Hz, 3H) MS (ESI) m/z 419.1 [M+H]⁺ SFC, retention time: 1.209; 1.515; 2.018; 2.453 min

Peak 3 was purified by Prep-HPLC (column: Phenomenex luna C18 150*25 mm*10 um; mobile phase: [water (FA)-ACN]; B %: 27%-57%, 10 min). The desired fraction was collected and lyophilized to afford (3S)—N-(2-(2,6-dioxopiperidin-3-yl)-1-oxoisoindolin-5-yl)-3-methylindoline-1-carboxamide. (12.87 mg, 30.8 umol, 12% yield) as a white solid.

¹H NMR (400 MHz, DMSO-d₆) δ=10.97 (br d, J=1.5 Hz, 1H), 8.85 (s, 1H), 7.92 (s, 1H), 7.87 (d, J=8.3 Hz, 1H), 7.70-7.60 (m, 2H), 7.24 (br d, J=7.4 Hz, 1H), 7.15 (br t, J=7.7 Hz, 1H), 7.01-6.91 (m, 1H), 5.09 (br dd, J=4.8, 13.1 Hz, 1H), 4.47-4.40 (m, 1H), 4.39-4.26 (m, 2H), 3.71 (dd, J=6.9, 9.9 Hz, 1H), 3.52 (br dd, J=7.1, 15.8 Hz, 1H), 2.98-2.85 (m, 1H), 2.60 (br d, J=17.5 Hz, 1H), 2.39 (br dd, J=4.2, 13.2 Hz, 1H), 2.01 (br dd, J=5.2, 10.4 Hz, 1H), 1.32 (d, J=6.8 Hz, 3H) MS (ESI) m/z 419.0 [M+H]⁺ SFC, retention time: 1.965; 2.429 min

Peak 4 was purified by Prep-HPLC (column: Phenomenex luna C18 150*25 mm*10 um; mobile phase: [water (FA)-ACN]; B %: 29%-59%, 10 min). The desired fraction was collected and lyophilized to afford (3S)—N-(2-(2,6-dioxopiperidin-3-yl)-1-oxoisoindolin-5-yl)-3-methylindoline-1-carboxamide. (8.17 mg, 19.52 umol, 6% yield) as a white solid.

¹H NMR (400 MHz, DMSO-d₆) δ=11.07-10.87 (m, 1H), 8.85 (s, 1H), 7.92 (s, 1H), 7.87 (d, J=8.0 Hz, 1H), 7.69-7.62 (m, 2H), 7.24 (d, J=7.3 Hz, 1H), 7.16 (t, J=7.7 Hz, 1H), 6.99-6.92 (m, 1H), 5.09 (dd, J=5.1, 13.3 Hz, 1H), 4.47-4.40 (m, 1H), 4.39-4.26 (m, 2H), 3.71 (dd, J=6.9, 10.0 Hz, 1H), 3.56-3.49 (m, 1H), 2.97-2.86 (m, 1H), 2.67-2.62 (m, 1H), 2.39 (br dd, J=4.5, 12.9 Hz, 1H), 2.04-1.95 (m, 1H), 1.32 (d, J=6.9 Hz, 3H) MS (ESI) m/z 418.9 [M+H]⁺ SFC, retention time: 2.023; 2.430 min

### Example 9. Synthesis of Compound 109

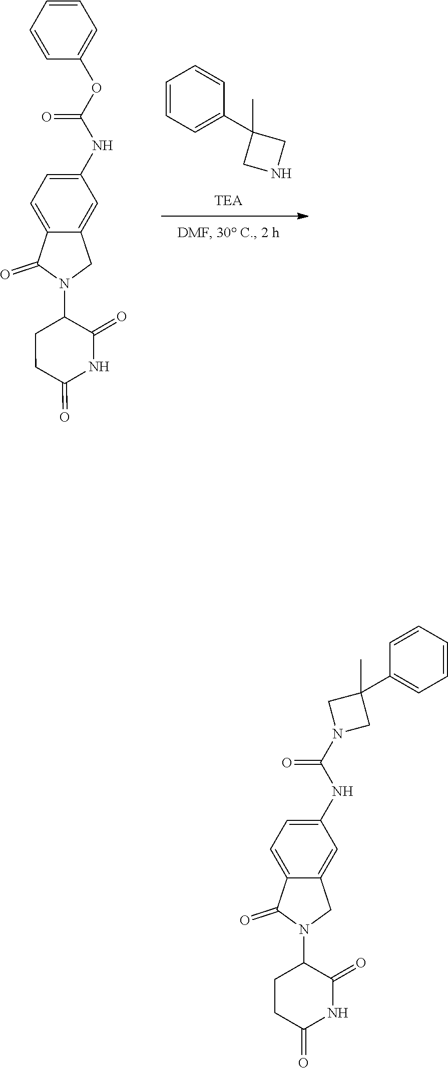 [SMILES: O=C1CCC(N2Cc3cc(NC(=O)Oc4ccccc4)ccc3C2=O)C(=O)N1 ; CC1(c2ccccc2)CNC1 ; CC1(c2ccccc2)CN(C(=O)Nc2ccc3c(c2)CN(C2CCC(=O)NC2=O)C3=O)C1]

To a mixture of phenyl (2-(2,6-dioxopiperidin-3-yl)-1-oxoisoindolin-5-yl)carbamate (150 mg, 395 umol, 1.10 eq) and 3-methyl-3-phenyl-azetidine (52.9 mg, 359 umol, 1.00 eq) in dimethyformamide (2.00 mL) was added triethylamine (109 mg, 1.08 mmol, 150 μL, 3.00 eq). The mixture was stirred at 30° C. for 2 h. The mixture was filtered. The filtrate was purified by reverse phase chromatography (column: spherical C18, 20-45 um, 100 Å, SW 80, mobile phase: [water (0.1% Formic Acid)-ACN) and lyophilized to give N-(2-(2,6-dioxopiperidin-3-yl)-1-oxoisoindolin-5-yl)-3-methyl-3-phenylazetidine-1-carboxamide (26.82 mg, 65.8 umol, 18% yield) as a white solid.

¹H NMR (400 MHz, DMSO-d₆) δ=10.94 (s, 1H), 8.84 (s, 1H), 7.84 (s, 1H), 7.58 (s, 2H), 7.40-7.35 (m, 2H), 7.33-7.30 (m, 2H), 7.27-7.23 (m, 1H), 5.06 (dd, J=5.2, 13.2 Hz, 1H), 4.42-4.36 (m, 1H), 4.27-4.20 (m, 3H), 4.04 (d, J=8.0 Hz, 2H), 2.94-2.85 (m, 1H), 2.61-2.56 (m, 1H), 2.38 (br d, J=4.6 Hz, 1H), 2.01-1.93 (m, 1H), 1.60 (s, 3H).

### Example 10. Synthesis of Compound 108

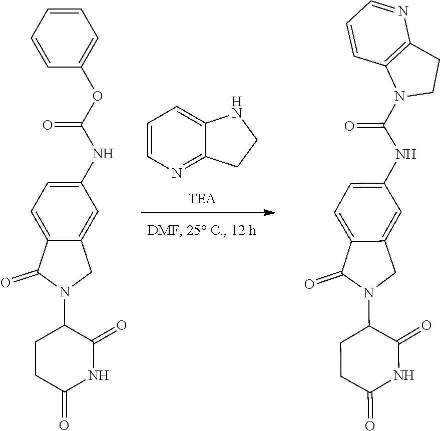 [SMILES: O=C1CCC(N2Cc3cc(NC(=O)Oc4ccccc4)ccc3C2=O)C(=O)N1 ; c1cnc2c(c1)NCC2 ; O=C1CCC(N2Cc3cc(NC(=O)N4CCc5ncccc54)ccc3C2=O)C(=O)N1]

To a solution of phenyl (2-(2,6-dioxopiperidin-3-yl)-1-oxoisoindolin-5-yl)carbamate (100 mg, 263 umol, 1.00 eq) in N,N-dimethyl formamide (1.00 mL) was added 2,3-dihydro-1H-pyrrolo[3,2-b]pyridine (34.8 mg, 289 umol, 1.10 eq) and triethylamine (80.0 mg, 790 umol, 110 μL, 3.00 eq). The solution was stirred at 25° C. for 12 h. The solution was adjust pH to 5-6 by formic acid (0.2 mL) and filtered. The filtrate was purified by prep-HPLC (column: Phenomenex luna C18 150*25 mm*10 um; mobile phase: [water (FA)-ACN]; B %: 0%-26%, 9 min) and lyophilized to afford N-(2-(2,6-dioxopiperidin-3-yl)-1-oxoisoindolin-5-yl)-2,3-dihydro-1H-pyrrolo[3,2-b]pyridine-1-carboxamide (9.15 mg, 22.3 umol, 8% yield, 99% purity, formate) as a white solid.

¹H NMR (400 MHz, DMSO-d₆) δ=10.99 (s, 1H), 8.98 (s, 1H), 8.26 (s, 0.2H), 8.09-8.02 (m, 2H), 7.93 (s, 1H), 7.67 (s, 2H), 7.17-7.11 (m, 1H), 5.10 (dd, J=5.1, 13.3 Hz, 1H), 4.47-4.42 (m, 1H), 4.33-4.28 (m, 1H), 4.22 (t, J=8.8 Hz, 2H), 3.26 (br s, 2H), 2.94-2.89 (m, 1H), 2.63 (br d, J=2.5 Hz, 1H), 2.39 (br dd, J=4.5, 13.3 Hz, 1H), 2.05-1.98 (m, 1H).

### Example 11. Synthesis of Compound 110

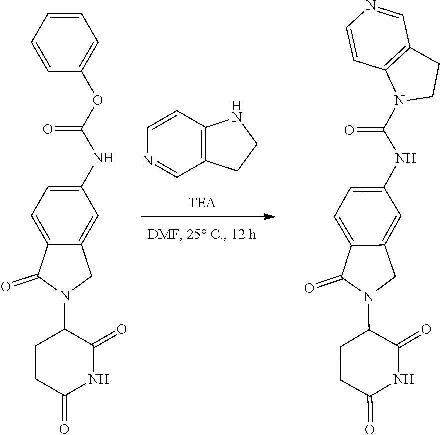 [SMILES: O=C1CCC(N2Cc3cc(NC(=O)Oc4ccccc4)ccc3C2=O)C(=O)N1 ; c1cc2c(cn1)CCN2 ; O=C1CCC(N2Cc3cc(NC(=O)N4CCc5cnccc54)ccc3C2=O)C(=O)N1]

To a solution of 2,3-dihydro-1H-pyrrolo[3,2-c]pyridine (50.0 mg, 416 umol, 1.00 eq) and phenyl (2-(2,6-dioxopiperidin-3-yl)-1-oxoisoindolin-5-yl)carbamate (158 mg, 416 umol, 1.00 eq) in N,N-dimethyl formamide (2.00 mL) was added triethylamine (126 mg, 1.25 mmol, 174 μL, 3.00 eq). The mixture was stirred at 25° C. for 12 h. The mixture was filtered. The filtrate was purified by prep-HPLC (column: Phenomenex luna C18 150*25 mm*10 um; mobile phase: [water (FA)-ACN]; B %: 0%-21%, 9 min) and further purified by prep-HPLC (column: Phenomenex luna C18 150*25 mm*10 um; mobile phase: [water (FA)-ACN]; B %: 0%-26%, 10 min). The desired fraction was collected and lyophilized to afford N-(2-(2,6-dioxopiperidin-3-yl)-1-oxoisoindolin-5-yl)-2,3-dihydro-1H-pyrrolo[3,2-c]pyridine-1-carboxamide (17.02 mg, 39.9 umol, 9% yield, 95% purity) as a white solid.

¹H NMR (400 MHz, DMSO-d₆) δ=10.97 (br s, 1H), 9.04 (s, 1H), 8.32 (s, 1H), 8.27 (d, J=5.4 Hz, 1H), 8.23 (s, 1H), 7.92 (s, 1H), 7.73 (d, J=5.4 Hz, 1H), 7.67 (d, J=1.0 Hz, 2H), 5.09 (dd, J=5.2, 13.4 Hz, 1H), 4.49-4.40 (m, 1H), 4.34-4.28 (m, 1H), 4.22 (t, J=8.8 Hz, 2H), 3.24 (br s, 2H), 2.93-2.87 (m, 1H), 2.64-2.61 (m, 1H), 2.39 (dd, J=4.3, 12.7 Hz, 1H), 2.06-1.95 (m, 1H).

### Example 12. Synthesis of Compound 105

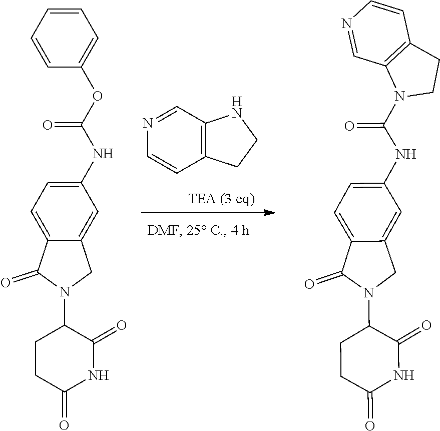 [SMILES: O=C1CCC(N2Cc3cc(NC(=O)Oc4ccccc4)ccc3C2=O)C(=O)N1 ; c1cc2c(cn1)NCC2 ; O=C1CCC(N2Cc3cc(NC(=O)N4CCc5ccncc54)ccc3C2=O)C(=O)N1]

To a solution of phenyl (2-(2,6-dioxopiperidin-3-yl)-1-oxoisoindolin-5-yl)carbamate (100 mg, 264 umol, 1.00 eq), 2,3-dihydro-1H-pyrrolo[2,3-c]pyridine (34.8 mg, 290 umol, 1.10 eq) in N,N-dimethyl formamide (2.00 mL) was added triethylamine (80.0 mg, 791 umol, 110 μL, 3.00 eq). The mixture was stirred at 25° C. for 4 h. The pH of the mixture was adjusted to pH=5-6 by formic acid and the mixture was filtered. The filtrate was purified by Prep-HPLC (column: Phenomenex luna C18 150*25 mm*10 um; mobile phase: [water (formic acid)-acetonitrile]; B %: 1%-20%, 10 min) and lyophilized to afford N-(2-(2,6-dioxopiperidin-3-yl)-1-oxoisoindolin-5-yl)-2,3-dihydro-1H-pyrrolo[2,3-c]pyridine-1-carboxamide (11.7 mg, 28.9 umol, 11% yield) yield, formate) as a yellow solid.

¹H NMR (400 MHz, DMSO-d₆) δ=10.97 (s, 1H), 9.03 (s, 1H), 8.96 (s, 1H), 8.20 (s, 0.1H), 8.15 (d, J=4.6 Hz, 1H), 7.94 (s, 1H), 7.67 (s, 2H), 7.29 (d, J=4.6 Hz, 1H), 5.16-5.02 (m, 1H), 4.50-4.40 (m, 1H), 4.34-4.26 (m, 1H), 4.19 (t, J=8.7 Hz, 2H), 3.28-3.24 (m, 2H), 2.98-2.86 (m, 1H), 2.62 (br s, 1H), 2.44 (br d, J=4.1 Hz, 1H), 2.04-1.96 (m, 1H).

### Example 13. Synthesis of Compound 112

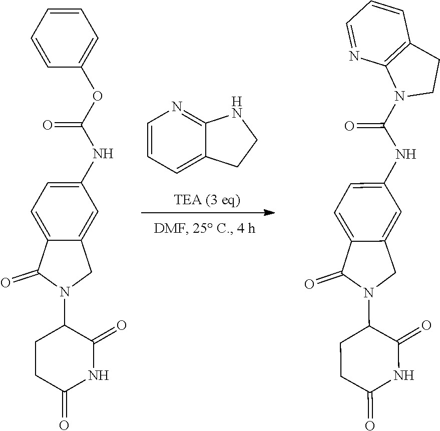 [SMILES: O=C1CCC(N2Cc3cc(NC(=O)Oc4ccccc4)ccc3C2=O)C(=O)N1 ; c1cnc2c(c1)CCN2 ; O=C1CCC(N2Cc3cc(NC(=O)N4CCc5cccnc54)ccc3C2=O)C(=O)N1]

To a solution of phenyl (2-(2,6-dioxopiperidin-3-yl)-1-oxoisoindolin-5-yl)carbamate (100 mg, 264 umol, 1.00 eq), 2,3-dihydro-1H-pyrrolo[2,3-b]pyridine (34.8 mg, 290 umol, 1.10 eq) in N,N-dimethyl formamide (2.00 mL) was added triethylamine (80.0 mg, 791 umol, 110 μL, 3.00 eq). The mixture was stirred at 25° C. for 4 h. The mixture was filtered. The filtrate was triturated with N,N-dimethyl formamide (2.00 mL) at 25° C. for 15 min and filtered. The filter cake was added water and lyophilized to afford N-(2-(2,6-dioxopiperidin-3-yl)-1-oxoisoindolin-5-yl)-2,3-dihydro-1H-pyrrolo[2,3-b]pyridine-1-carboxamide (59.9 mg, 148 umol, 56% yield) as a grey solid.

¹H NMR (400 MHz, DMSO-d₆) δ=11.87 (s, 1H), 11.00 (s, 1H), 8.16 (d, J=5.0 Hz, 1H), 7.94 (s, 1H), 7.78-7.62 (m, 3H), 7.01 (dd, J=5.4, 7.1 Hz, 1H), 5.10 (dd, J=5.1, 13.3 Hz, 1H), 4.50-4.40 (m, 1H), 4.36-4.27 (m, 1H), 4.07 (t, J=8.6 Hz, 2H), 3.13 (br t, J=8.5 Hz, 2H), 2.96-2.85 (m, 1H), 2.60 (br d, J=17.0 Hz, 1H), 2.40 (br dd, J=4.3, 13.3 Hz, 1H), 2.04-1.95 (m, 1H).

### Example 14. Synthesis of Compound 285

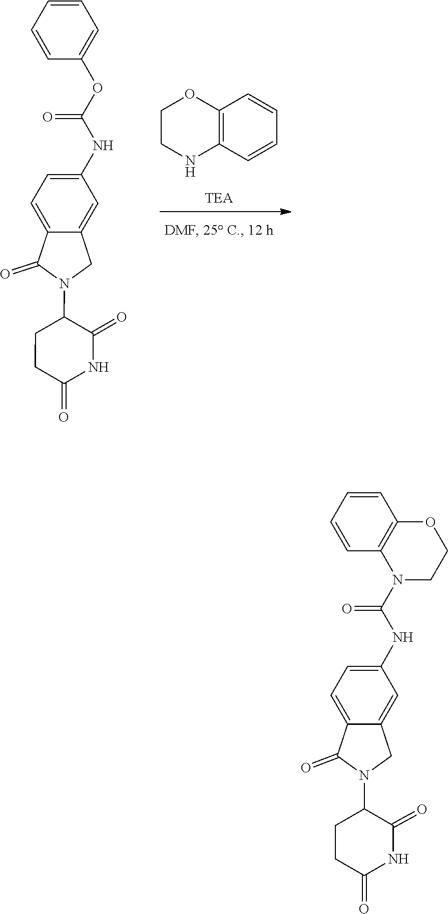 [SMILES: O=C1CCC(N2Cc3cc(NC(=O)Oc4ccccc4)ccc3C2=O)C(=O)N1 ; c1ccc2c(c1)NCCO2 ; O=C1CCC(N2Cc3cc(NC(=O)N4CCOc5ccccc54)ccc3C2=O)C(=O)N1]

To a solution of phenyl (2-(2,6-dioxopiperidin-3-yl)-1-oxoisoindolin-5-yl)carbamate (200 mg, 527 umol, 1.00 eq) in N,N-dimethyl formamide (2.00 mL) was added 3,4-dihydro-2H-1,4-benzoxazine (78.3 mg, 579 umol, 1.10 eq) and triethylamine (160 mg, 1.58 mmol, 220 μL, 3.00 eq). Then the mixture was stirred at 25° C. for 12 h. The mixture was adjusted pH to 5-6 by formic acid (0.1 mL) and filtered. The filtrate was purified by reversed phase column chromatography (C18, 40 g; condition: water/acetonitrile=1/0 to 0/1, 0.1% formic acid) and lyophilized to afford N-(2-(2,6-dioxopiperidin-3-yl)-1-oxoisoindolin-5-yl)-2H-benzo[b][1,4]oxazine-4(3H)-carboxamide (12.47 mg, 25.7 umol, 4% yield, 96% purity) as a gray solid.

¹H NMR (400 MHz, DMSO-d₆) δ=10.98 (br d, J=1.8 Hz, 1H), 9.49 (s, 1H), 8.25 (s, 0.6H), 7.80 (s, 1H), 7.70-7.61 (m, 1H), 7.59-7.48 (m, 2H), 7.01-6.95 (m, 1H), 6.92-6.84 (m, 2H), 5.09 (dd, J=5.1, 13.3 Hz, 1H), 4.47-4.39 (m, 1H), 4.29 (dd, J=5.1, 9.4 Hz, 3H), 3.90-3.85 (m, 2H), 2.96-2.87 (m, 1H), 2.65-2.59 (m, 1H), 2.41-2.33 (m, 1H), 2.05-1.96 (m, 1H).

### Example 15. Synthesis of Compound 286

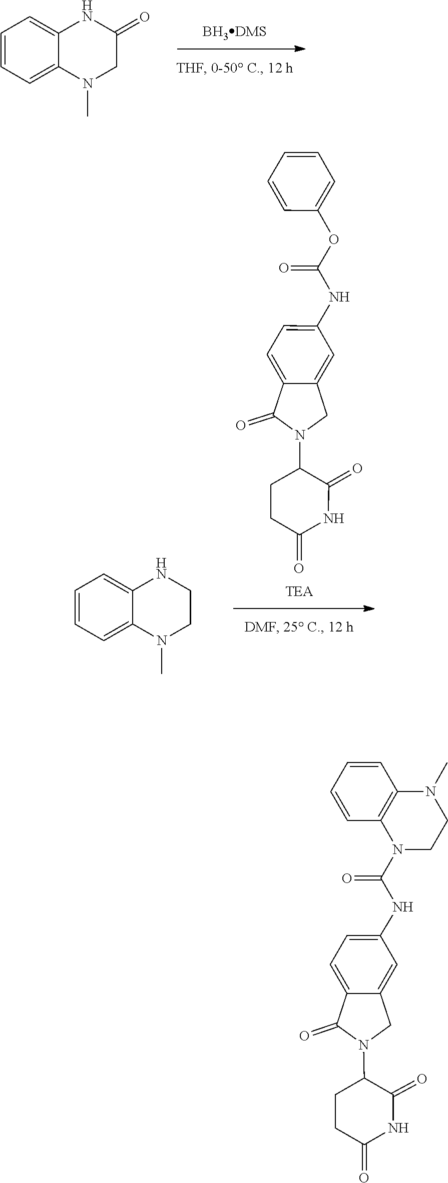 [SMILES: CN1CC(=O)Nc2ccccc21 ; O=C1CCC(N2Cc3cc(NC(=O)Oc4ccccc4)ccc3C2=O)C(=O)N1 ; CN1CCNc2ccccc21 ; CN1CCN(C(=O)Nc2ccc3c(c2)CN(C2CCC(=O)NC2=O)C3=O)c2ccccc21]

Step 1. To a mixture of 4-methyl-3,4-dihydroquinoxalin-2(11H)-one (900 mg, 5.55 mmol, 1.00 eq) in tetrahydrofuran (20.0 mL) was added borane dimethyl sulfide complex (10 M, 1.66 mL, 3.00 eq) dropwise at 0° C. The mixture was stirred at 50° C. for 12 h. The mixture was poured into methanol (200 mL), then the mixture was concentrated in vacuum. The residue was purified by silica gel chromatography (petroleum ether/ethyl acetate=5/1) to give 1-methyl-1,2,3,4-tetrahydroquinoxaline (300 mg, 2.02 mmol, 36% yield) as yellow oil.

¹H NMR (400 MHz, CDCl₃) δ=6.74-6.65 (m, 1H), 6.63-6.56 (m, 2H), 6.49 (dd, J=1.5, 7.5 Hz, 1H), 3.50 (br s, 2H), 3.31-3.26 (m, 2H), 2.89 (s, 3H).

Step 2. To a mixture of phenyl (2-(2,6-dioxopiperidin-3-yl)-1-oxoisoindolin-5-yl)carbamate (300 mg, 791 umol, 1.00 eq) and 1-methyl-1,2,3,4-tetrahydroquinoxaline (106 mg, 712 umol, 0.900 eq) in dimethyformamide (2.00 mL) was added triethylamine (240 mg, 2.37 mmol, 330 μL, 3.00 eq) dropwise. The mixture was stirred at 25° C. for 12 h. The mixture was adjusted PH<7 with formic acid (0.2 mL) and filtered. The filtrate was purified by reverse phase chromatography (column: spherical C18, 20-45 um, 100 Å, SW 80, mobile phase: [water (0.1% Formic Acid)-ACN) and lyophilized to give N-(2-(2,6-dioxopiperidin-3-yl)-1-oxoisoindolin-5-yl)-4-methyl-3,4-dihydroquinoxaline-1(2H)-carboxamide (29.91 mg, 68.3 umol, 9% yield, 99% purity) as a gray solid.

¹H NMR (400 MHz, DMSO-d₆) δ=10.96 (br s, 1H), 9.13 (s, 1H), 7.77 (s, 1H), 7.62-7.57 (m, 1H), 7.56-7.47 (m, 1H), 7.17 (d, J=7.9 Hz, 1H), 7.01-6.89 (m, 1H), 6.74 (d, J=8.1 Hz, 1H), 6.60 (t, J=7.5 Hz, 1H), 5.08 (br dd, J=5.0, 13.1 Hz, 1H), 4.45-4.33 (m, 1H), 4.31-4.22 (m, 1H), 3.78 (br t, J=4.6 Hz, 2H), 3.39-3.34 (m, 2H), 2.95-2.86 (m, 4H), 2.59 (br d, J=16.6 Hz, 1H), 2.37 (br dd, J=4.1, 13.3 Hz, 1H), 2.03-1.94 (m, 1H).

### Example 16. Synthesis of Compound 289

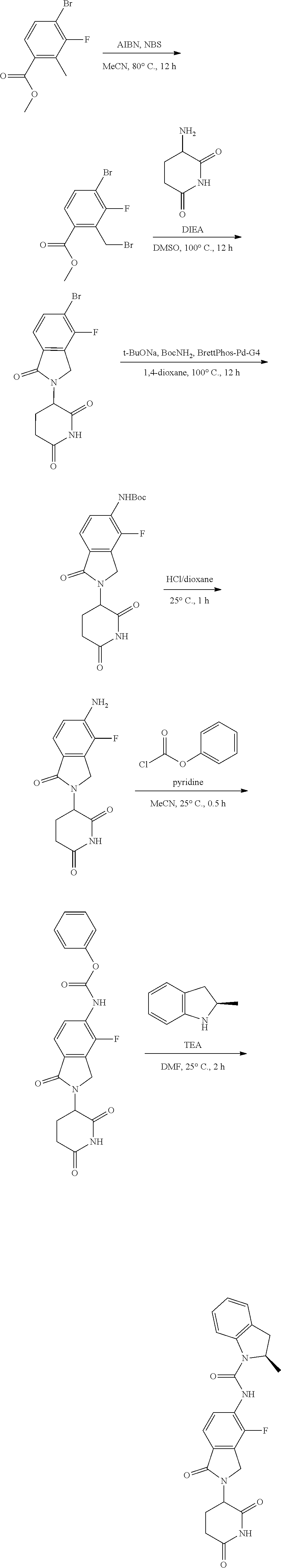

Step 1. A mixture of methyl 4-bromo-3-fluoro-2-methylbenzoate (300 mg, 1.21 mmol, 1.00 eq) in acetonitrile (10.0 mL) was added N-bromosuccinimide (324 mg, 1.82 mmol, 1.50 eq) and 2,2′-azobis(2-methylpropionitrile) (398 mg, 2.43 mmol, 2.00 eq). The mixture was stirred at 80° C. for 12 h. The mixture was concentrated in vacuum to give methyl 4-bromo-2-(bromomethyl)-3-fluoro-benzoate (700 mg, crude) as a yellow solid.

¹H NMR (400 MHz, DMSO-d₆) δ=7.90-7.83 (m, 1H), 7.68 (d, J=8.5 Hz, 1H), 4.98 (d, J=1.6 Hz, 2H), 3.89 (s, 3H).

Step 2. To a mixture of methyl 4-bromo-2-(bromomethyl)-3-fluorobenzoate (400 mg, 1.23 mmol, 1.00 eq) and 3-aminopiperidine-2,6-dione (202 mg, 1.23 mmol, 1.00 eq, hydrochloride) in dimethylsulfoxide (5.00 mL) was added N,N-diisopropylethylamine (476 mg, 3.68 mmol, 641 μL, 3.00 eq), then the mixture was stirred at 100° C. for 12 h. The reaction mixture was diluted with water (20 mL) and extracted with ethyl acetate (3×20 mL). The combined organic layers were washed with brine (20 mL), dried over sodium sulfate, filtered and concentrated under reduced pressure. The residue was purified by column chromatography (SiO₂, Petroleum ether/Ethyl acetate=0/1 to 1/0) and concentrated in vacuum to give 3-(5-bromo-4-fluoro-1-oxoisoindolin-2-yl)piperidine-2,6-dione (200 mg, 586 umol, 47% yield) as yellow oil.

¹H NMR (400 MHz, DMSO-d₆) δ=11.01 (s, 1H), 7.95-7.81 (m, 1H), 7.54 (d, J=8.0 Hz, 1H), 5.22-5.03 (m, 1H), 4.67-4.56 (m, 1H), 4.51-4.39 (m, 1H), 2.93-2.85 (m, 1H), 2.60 (br d, J=17.1 Hz, 1H), 2.45-2.38 (m, 1H), 2.03-1.97 (m, 1H).

Step 3. To a mixture of 3-(5-bromo-4-fluoro-1-oxo-isoindolin-2-yl)piperidine-2,6-dione (100 mg, 293 umol, 1.00 eq) and tert-butyl carbamate (103 mg, 879 umol, 3.00 eq) in dioxane (1.00 mL) was added sodium tert-butoxide (2.00 M, 440 μL, 3.00 eq). The mixture was added methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(ii) (27.0 mg, 29.3 umol, 0.100 eq) and stirred at 100° C. for 12 h under nitrogen. The mixture was concentrated in vacuum. The residue was dissolved in dimethyformamide (1 mL) and filtered. The filtrate was purified by reverse phase chromatography (column: spherical C18, 20-45 um, 100 Å, SW 40, mobile phase: [water (0.1% Formic Acid)-ACN) and lyophilized to give tert-butyl (2-(2,6-dioxopiperidin-3-yl)-4-fluoro-1-oxoisoindolin-5-yl)carbamate (30.0 mg, 79.5 umol, 13% yield) as a gray solid.

¹H NMR (400 MHz, DMSO-d₆) δ=10.99 (s, 1H), 9.37 (s, 1H), 7.82 (t, J=7.5 Hz, 1H), 7.52 (d, J=8.3 Hz, 1H), 5.09 (dd, J=5.2, 13.3 Hz, 1H), 4.53 (d, J=17.3 Hz, 1H), 4.40-4.31 (m, 1H), 2.96-2.84 (m, 1H), 2.67 (td, J=1.7, 3.6 Hz, 1H), 2.40 (br d, J=4.8 Hz, 1H), 2.04-1.98 (m, 1H), 1.48 (s, 9H).

Step 4. A mixture of tert-butyl (2-(2,6-dioxopiperidin-3-yl)-4-fluoro-1-oxoisoindolin-5-yl)carbamate (30.0 mg, 79.5 umol, 1.00 eq) in hydrochloric acid (4.00 M in dioxane, 5.00 mL) was stirred at 25° C. for 1 h. The mixture was concentrated in vacuum to give 3-(5-amino-4-fluoro-1-oxoisoindolin-2-yl)piperidine-2,6-dione (25.0 mg, crude) as a yellow solid. MS (ESI) m/z. 277.9 [M+H]⁺

Step 5. To a mixture of 3-(5-amino-4-fluoro-1-oxo-isoindolin-2-yl)piperidine-2,6-dione (25.0 mg, 90.2 umol, 1.00 eq) in acetonitrile (5.00 mL) was added pyridine (21.4 mg, 270 umol, 21.8 uL, 3.00 eq) and phenyl carbonochloridate (16.9 mg, 108 umol, 13.6 uL, 1.20 eq) dropwise at 0° C. The mixture was stirred at 25° C. for 0.5 h. The mixture was concentrated in vacuum. The crude product was purified by reversed-phase HPLC (column: spherical C18, 20-45 um, 100 Å, SW 25, mobile phase: [water (0.1% Formic Acid)-ACN) and lyophilized to give phenyl (2-(2,6-dioxopiperidin-3-yl)-4-fluoro-1-oxoisoindolin-5-yl)carbamate (12.0 mg, 30.2 umol, 33% yield) as a yellow solid. MS (ESI) m/z. 397.9 [M+H]⁺

Step 6. To a mixture of phenyl (2-(2,6-dioxopiperidin-3-yl)-4-fluoro-1-oxoisoindolin-5-yl)carbamate (12.0 mg, 30.2 umol, 1.00 eq) and (2R)-2-methylindoline (4.02 mg, 30.2 umol, 1.00 eq) in dimethyformamide (0.500 mL) was added triethylamine (9.17 mg, 90.6 umol, 12.6 uL, 3.00 eq). The mixture was stirred at 25° C. for 2 h. The mixture was filtered. The filtrate was purified by prep-HPLC (column: Phenomenex luna C18 150*25 mm*10 um; mobile phase: [water (FA)-ACN]; B %: 31%-61%, 58 min) and lyophilized to give (2R)—N-(2-(2,6-dioxopiperidin-3-yl)-4-fluoro-1-oxoisoindolin-5-yl)-2-methylindoline-1-carboxamide (2.80 mg, 6.42 umol, 21.2% yield, formate) as an off-white solid.

¹H NMR (400 MHz, DMSO-d₆) δ=11.10-10.91 (m, 1H), 8.82 (s, 1H), 7.82 (d, J=8.0 Hz, 1H), 7.72-7.62 (m, 1H), 7.56 (d, J=8.0 Hz, 1H), 7.24 (d, J=7.1 Hz, 1H), 7.14 (t, J=7.8 Hz, 1H), 6.94 (t, J=7.4 Hz, 1H), 5.12 (dd, J=5.2, 13.2 Hz, 1H), 4.87-4.79 (m, 1H), 4.62-4.55 (m, 1H), 4.44-4.36 (m, 1H), 3.41 (br d, J=6.8 Hz, 1H), 2.95-2.87 (m, 1H), 2.74-2.65 (m, 1H), 2.62-2.58 (m, 1H), 2.43-2.38 (m, 1H), 2.04-2.00 (m, 1H), 1.28 (d, J=6.1 Hz, 3H).

### Example 17. Synthesis of Compound 288

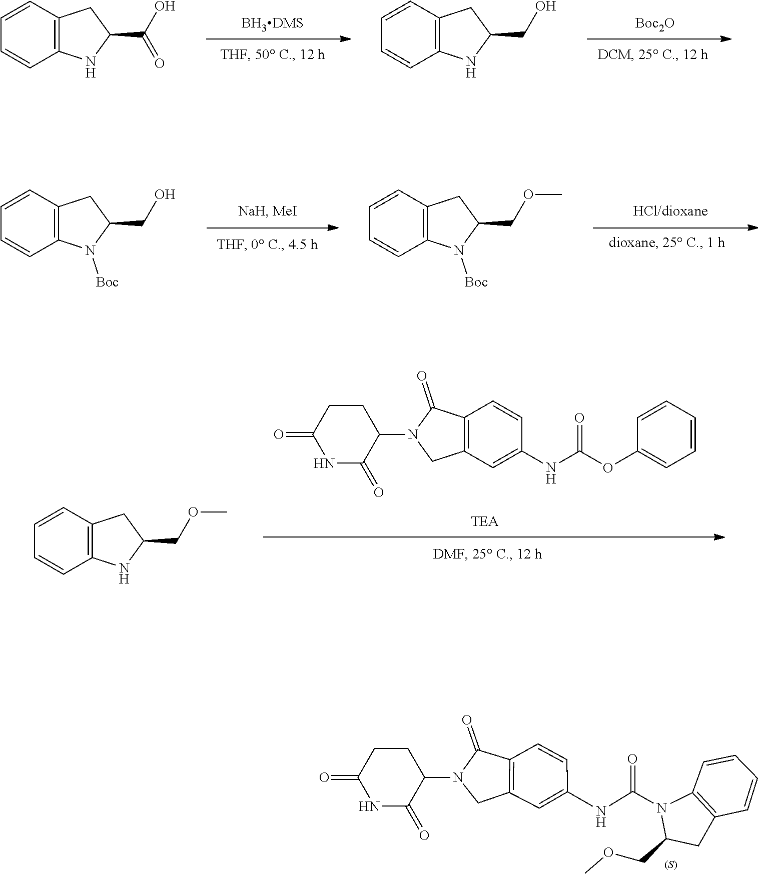

Step 1. To a solution of (S)-indolin-2-ylmethanol (5.00 g, 30.6 mmol, 1.00 eq) in tetrahydrofuran (50.0 mL) was added borane dimethyl sulfide complex (10.0 M, 6.13 mL, 2.00 eq) at 0° C. The mixture was stirred at 50° C. for 12 h. After being cooled to room temperature, the mixture was added methyl alcohol (10.0 mL), then the mixture was stirred at 50° C. for 0.5 h. The mixture was concentrated under reduced pressure to give a residue. The crude product was purified by column chromatography on silica gel eluted with petroleum ether/ethyl acetate=50/1 to 0/1 to afford (S)-indolin-2-ylmethanol (4.40 g, 29.5 mmol, 96% yield) as a black solid.

¹H NMR (400 MHz, DMSO-d₆) δ=6.96 (d, J=7.1 Hz, 1H), 6.87 (t, J=7.5 Hz, 1H), 6.51-6.44 (m, 2H), 5.52 (br s, 1H), 4.71 (br s, 1H), 3.82-3.73 (m, 1H), 3.36-3.30 (m, 2H), 2.97 (dd, J=9.1, 15.9 Hz, 1H), 2.63 (dd, J=6.8, 15.8 Hz, 1H)

Step 2. To a solution of (S)-indolin-2-ylmethanol (500 mg, 3.35 mmol, 1.00 eq) in dichloromethane (3.00 mL) was added di-tert-butyl dicarbonate (805 mg, 3.69 mmol, 847 μL, 1.10 eq). The mixture was stirred at 25° C. for 12 h. After being cooled to room temperature, the mixture was diluted with water (200 mL) and extracted with ethyl acetate (3×100 mL). The organic layers were collected and was washed with brine (50.0 mL), dried over anhydrous sodium sulfate and evaporated to dryness. The crude product was purified by column chromatography on silica gel eluted with petroleum ether/ethyl acetate=50/1 to 0/1 to afford (S)-tert-butyl 2-(methoxymethyl)indoline-1-carboxylate (800 mg, 3.21 mmol, 95% yield) as yellow oil.

¹H NMR (400 MHz, DMSO-d₆) δ=7.55 (br d, J=7.9 Hz, 1H), 7.19-7.07 (m, 2H), 6.93-6.87 (m, 1H), 4.86 (t, J=5.6 Hz, 1H), 3.60-3.52 (m, 1H), 3.38-3.33 (m, 1H), 3.20 (br dd, J=9.9, 16.4 Hz, 1H), 2.98 (br dd, J=1.6, 16.4 Hz, 1H), 1.50 (s, 9H).

Step 3. To a solution of tert-butyl (2S)-2-(hydroxymethyl)indoline-1-carboxylate (200 mg, 802 umol, 1.00 eq) in tetrahydrofuran (5.00 mL) was added sodium hydride (35.3 mg, 882 umol, 60% purity, 1.10 eq) at 0° C. The mixture was stirred at 0° C. for 0.5 h. Then methyl iodide (456 mg, 3.21 mmol, 0.200 mL, 4.00 eq) was added into the mixture, the mixture was stirred at 0° C. for 4 h. The mixture was quenched with saturated ammonium chloride aqueous solution (50 mL), then extracted with ethyl acetate (3×50 mL). The organic layers were collected and dried over anhydrous sodium sulfate and evaporated to dryness. The crude product was purified by column chromatography on silica gel eluted with petroleum ether/ethyl acetate=50/1 to 0/1 to afford (S)-tert-butyl 2-(methoxymethyl)indoline-1-carboxylate (200 mg, 759 umol, 94% yield) as yellow oil.

¹H NMR (400 MHz, DMSO-d₆) δ=7.67-7.47 (m, 1H), 7.18 (d, J=7.3 Hz, 1H), 7.12 (t, J=7.8 Hz, 1H), 6.96-6.89 (m, 1H), 4.54-4.44 (m, 1H), 3.45 (d, J=3.6 Hz, 1H), 3.38 (br s, 1H), 3.29-3.27 (m, 1H), 3.25 (s, 3H), 2.92-2.86 (m, 1H), 1.51 (s, 9H)

Step 4. A solution of tert-butyl (S)-tert-butyl 2-(methoxymethyl)indoline-1-carboxylate (200 mg, 759 umol, 1.00 eq) in hydrochloric acid/dioxane (3.00 mL) was stirred at 25° C. for 2 h. The mixture was concentrated under reduced pressure to afford (S)-2-(methoxymethyl)indoline (200 mg, crude) as a yellow solid.

Step 5. To a solution of (S)-2-(methoxymethyl)indoline (150 mg, 919 umol, 1.00 eq), phenyl (2-(2,6-dioxopiperidin-3-yl)-1-oxoisoindolin-5-yl)carbamate (349 mg, 919 umol, 1.00 eq) in N,N-dimethyl formamide (3.00 mL) was added triethylamine (279 mg, 2.76 mmol, 384 μL, 3.00 eq). The mixture was stirred at 25° C. for 12 h. The mixture was filtered. The filter liquor was purified by prep-HPLC (column: Phenomenex luna C18 150*25 mm*10 um; mobile phase: [water (FA)-ACN]; B %: 30%-60%, 9 min). The desired fraction was collected and lyophilized to afford (2S)—N-(2-(2,6-dioxopiperidin-3-yl)-1-oxoisoindolin-5-yl)-2-(methoxymethyl)indoline-1-carboxamide (33.35 mg, 73.6 umol, 8% yield, 99% purity) as an off-white solid.

¹H NMR (400 MHz, DMSO-d₆) δ=10.95 (br s, 1H), 9.15 (s, 1H), 7.81 (s, 1H), 7.76 (d, J=8.0 Hz, 1H), 7.65 (d, J=8.4 Hz, 1H), 7.53 (br d, J=8.6 Hz, 1H), 7.22 (d, J=7.3 Hz, 1H), 7.15 (t, J=7.7 Hz, 1H), 6.94 (t, J=7.4 Hz, 1H), 5.09 (dd, J=5.1, 13.3 Hz, 1H), 4.98-4.89 (m, 1H), 4.47-4.39 (m, 1H), 4.34-4.26 (m, 1H), 3.49-3.47 (m, 1H), 3.35 (s, 3H), 2.97-2.90 (m, 1H), 2.90-2.85 (m, 1H), 2.82 (s, 1H), 2.65 (br d, J=17.6 Hz, 1H), 2.58 (br d, J=3.4 Hz, 1H), 2.37 (br d, J=3.8 Hz, 1H), 2.03-1.97 (m, 1H)

### Example 18. Synthesis of Compound 216

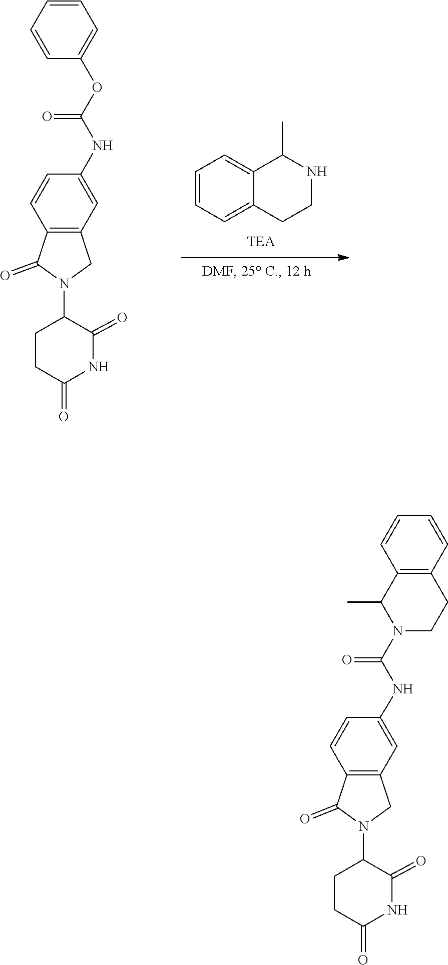 [SMILES: O=C1CCC(N2Cc3cc(NC(=O)Oc4ccccc4)ccc3C2=O)C(=O)N1 ; CC1NCCc2ccccc21 ; CC1c2ccccc2CCN1C(=O)Nc1ccc2c(c1)CN(C1CCC(=O)NC1=O)C2=O]

Step 1. To a solution of phenyl (2-(2,6-dioxopiperidin-3-yl)-1-oxoisoindolin-5-yl)carbamate (200 mg, 527 umol, 1.00 eq) and 1-methyl-1,2,3,4-tetrahydroisoquinoline (77.6 mg, 527 umol, 1.00 eq) in N,N-dimethyl formamide (2.00 mL) was added triethylamine (160 mg, 1.58 mmol, 220 μL, 3.00 eq). The mixture was stirred at 25° C. for 12 h. The mixture was filtered. The filtrate was purified by prep-HPLC (column: Phenomenex luna C18 150*25 mm*10 um; mobile phase: [water (FA)-ACN]; B %: 25%-55%, 10 min). The desired fraction was collected and lyophilized to afford N-(2-(2,6-dioxopiperidin-3-yl)-1-oxoisoindolin-5-yl)-1-methyl-3,4-dihydroisoquinoline-2(1H)-carboxamide (13.34 mg, 30.5 umol, 5% yield, 99% purity) as a white solid.

¹H NMR (400 MHz, DMSO-d₆) δ=11.04-10.89 (m, 1H), 8.89 (s, 1H), 7.81 (s, 1H), 7.62-7.53 (m, 2H), 7.24-7.19 (m, 2H), 7.19-7.15 (m, 2H), 5.43-5.32 (m, 1H), 5.11-5.04 (m, 1H), 4.43-4.36 (m, 1H), 4.29-4.22 (m, 1H), 4.21-4.13 (m, 1H), 2.92-2.89 (m, 1H), 2.87 (br d, J=5.5 Hz, 1H), 2.84-2.80 (m, 1H), 2.61 (br s, 1H), 2.37 (br dd, J=4.3, 13.3 Hz, 1H), 2.34-2.31 (m, 1H), 2.02-1.94 (m, 1H), 1.44 (d, J=6.8 Hz, 3H).

### Example 19. Synthesis of Compound 191

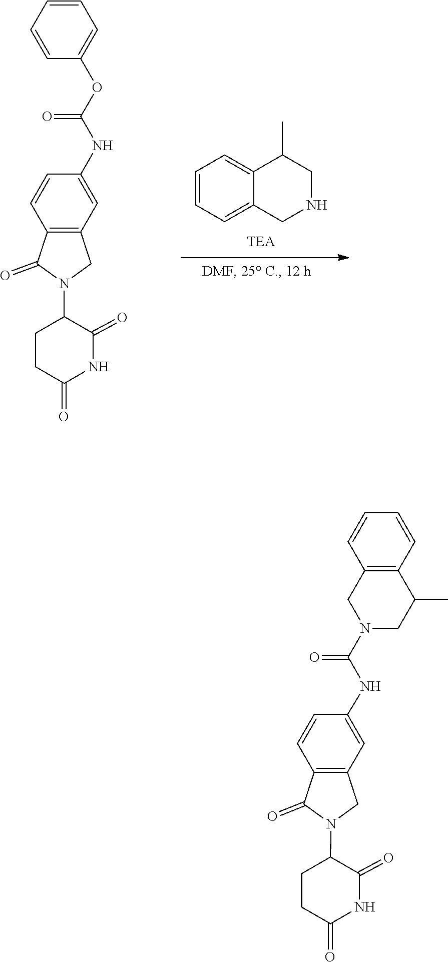 [SMILES: O=C1CCC(N2Cc3cc(NC(=O)Oc4ccccc4)ccc3C2=O)C(=O)N1 ; CC1CNCc2ccccc21 ; CC1CN(C(=O)Nc2ccc3c(c2)CN(C2CCC(=O)NC2=O)C3=O)Cc2ccccc21]

Step 1. To a solution of phenyl (2-(2,6-dioxopiperidin-3-yl)-1-oxoisoindolin-5-yl)carbamate (200 mg, 527 umol, 1.00 eq) in N,N-dimethyl formamide (2.00 mL) was added 4-methyl-1,2,3,4-tetrahydroisoquinoline (106 mg, 579 umol, 1.10 eq, hydrochloride) and triethylamine (160 mg, 1.58 mmol, 220 μL, 3.00 eq). Then the mixture was stirred at 25° C. for 12 h. The solution was adjusted pH<7 by formic acid and filtered. The filtrate was purified by prep-HPLC (column: Phenomenex luna C18 150*25 mm*10 um; mobile phase: [water (FA)-ACN]; B %: 25%-55%, 10 min) and lyophilized to afford N-(2-(2,6-dioxopiperidin-3-yl)-1-oxoisoindolin-5-yl)-4-methyl-3,4-dihydroisoquinoline-2(1H)-carboxamide (17.88 mg, 40.9 umol, 7% yield, 99% purity) as a white solid.

¹H NMR (400 MHz, DMSO-d₆) δ=10.96 (s, 1H), 8.90 (s, 1H), 7.82 (s, 1H), 7.64-7.54 (m, 2H), 7.31-7.27 (m, 1H), 7.26-7.11 (m, 3H), 5.08 (dd, J=5.1, 13.3 Hz, 1H), 4.79 (d, J=16.3 Hz, 1H), 4.65-4.56 (m, 1H), 4.43-4.38 (m, 1H), 4.29-4.24 (m, 1H), 3.71 (br d, J=8.6 Hz, 1H), 3.57 (br s, 1H), 3.03 (br dd, J=6.3, 11.3 Hz, 1H), 2.94-2.89 (m, 1H), 2.62-2.58 (m, 1H), 2.44-2.34 (m, 1H), 2.05-1.95 (m, 1H), 1.26 (d, J=6.9 Hz, 3H).

### Example 20. Synthesis of Compound 287

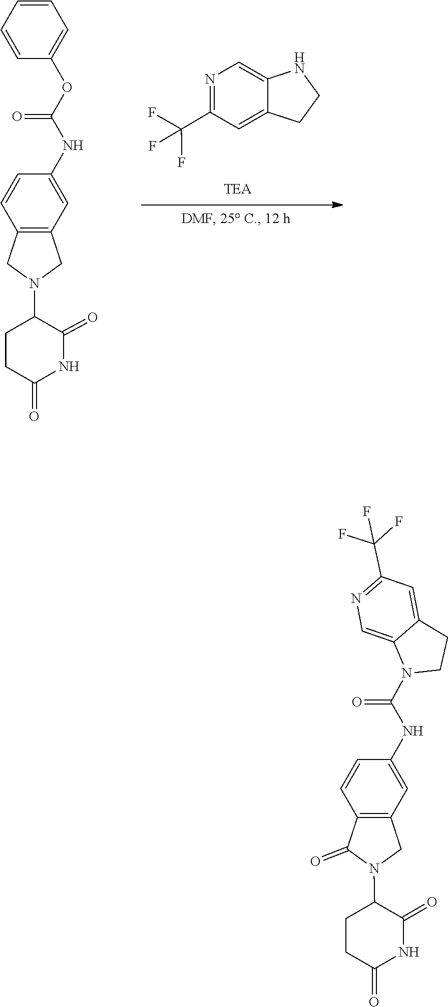 [SMILES: O=C1CCC(N2Cc3ccc(NC(=O)Oc4ccccc4)cc3C2)C(=O)N1 ; FC(F)(F)c1cc2c(cn1)NCC2 ; O=C1CCC(N2Cc3cc(NC(=O)N4CCc5cc(C(F)(F)F)ncc54)ccc3C2=O)C(=O)N1]

To a solution of phenyl (2-(2,6-dioxopiperidin-3-yl)-1-oxoisoindolin-5-yl)carbamate (300 mg, 790 umol, 2.50 eq) in N,N-dimethylformamide (3.00 mL) was added 5-(trifluoromethyl)-2,3-dihydro-1H-pyrrolo[2,3-c]pyridine (59.4 mg, 316 umol, 0.900 eq), triethylamin (96.0 mg, 948 umol, 132 μL, 3.00 eq). Then the mixture was stirred at 25° C. for 12 h. The solution was adjusted pH<7 by formic acid (0.1 mL) and filtered. The filtrate was purified by reversed phase column chromatography (C18, 80 g; condition: water/acetonitrile=1/0 to 0/1, 0.1% formic acid) and lyophilized to afford N-(2-(2,6-dioxopiperidin-3-yl)-1-oxoisoindolin-5-yl)-5-(trifluoromethyl)-2,3-dihydro-1H-pyrrolo[2,3-c]pyridine-1-carboxamide (14.26 mg, 29.8 umol, 9% yield, 99% purity) as a white solid.

¹H NMR (400 MHz, DMSO-d₆) δ=11.00 (br s, 1H), 9.26-9.05 (m, 2H), 8.47 (s, 0.1H), 7.97 (s, 1H), 7.82 (s, 1H), 7.71 (s, 2H), 5.12 (dd, J=5.1, 13.3 Hz, 1H), 4.51-4.43 (m, 1H), 4.37-4.28 (m, 3H), 3.40-3.38 (m, 2H), 2.97-2.88 (m, 1H), 2.66-2.60 (m, 1H), 2.41 (br dd, J=4.3, 13.2 Hz, 1H), 2.08-1.98 (m, 1H).

### Example 21. Synthesis of Compound 124

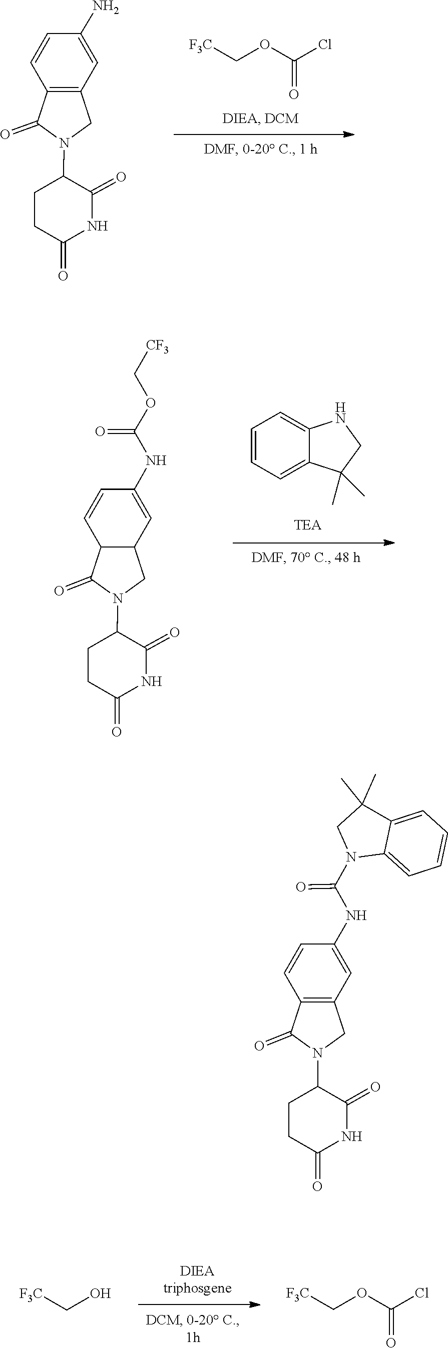

Step 1. To a solution of 2,2,2-trifluoroethanol (1.00 g, 10.0 mmol, 719 μL, 1.00 eq) in dichloromethane (15.0 mL) was added bis(trichloromethyl)carbonate (4.75 g, 15.9 mmol, 1.60 eq) and N,N-diisopropylethylamine (2.58 g, 19.9 mmol, 3.48 mL, 2.00 eq) at 0° C. The reaction mixture was stirred at 20° C. for 1 h. The reaction mixture was concentrated in vacuum to give 2,2,2-trifluoroethyl carbonochloridate (1.40 g, crude) as a yellow solid.

Step 2. To a solution of 3-(5-amino-1-oxoisoindolin-2-yl)piperidine-2,6-dione (1.00 g, 3.86 mmol, 1.00 eq) in dimethyl formamide (3.00 mL) was added 2,2,2-trifluoroethyl carbonochloridate (689 mg, 4.24 mmol, 1.10 eq) dissolved in dichloromethane (10.0 mL) and N,N-diisopropylethylamine (997 mg, 7.71 mmol, 1.34 mL, 2.00 eq) at 0° C. The reaction mixture was stirred at 20° C. for 1 h. The reaction mixture was concentrated in vacuum to give a residue, which was purified by reversed-phase HPLC (column: spherical C18, 20-45 um, 100 Å, SW 120, mobile phase: [water (0.1% Formic Acid)-acetonitrile) to give 2,2,2-trifluoroethyl (2-(2,6-dioxopiperidin-3-yl)-1-oxoisoindolin-5-yl)carbamate (430 mg, 1.12 mmol, 28% yield) as a white solid.

¹H NMR (400 MHz, DMSO-d₆) δ=10.97 (br s, 1H), 10.52 (s, 1H), 7.79 (s, 1H), 7.68 (d, J=8.4 Hz, 1H), 7.55 (dd, J=1.5, 8.3 Hz, 1H), 5.08 (dd, J=5.1, 13.3 Hz, 1H), 4.83 (q, J=9.0 Hz, 2H), 4.47-4.39 (m, 1H), 4.33-4.25 (m, 1H), 2.97-2.85 (m, 1H), 2.64-2.55 (m, 1H), 2.37 (dq, J=4.3, 13.2 Hz, 1H), 1.99 (s, 1H).

Step 3. To a solution of 2,2,2-trifluoroethyl (2-(2,6-dioxopiperidin-3-yl)-1-oxoisoindolin-5-yl)carbamate (100 mg, 259 umol, 1.00 eq), triethylamine (78.7 mg, 778 umol, 108 μL, 3.00 eq) in dimethyl formamide (3.00 mL) was added 3,3-dimethylindoline (49.6 mg, 337 umol, 1.30 eq). The mixture was stirred at 70° C. for 48 h. The mixture was filtered to give a filter liquor which was purified by Prep-HPLC (column: Phenomenex luna C18 150*25 mm*10 um; mobile phase: [water (formic acid)-acetonitrile]; B %: 32%-62%, 10 min) to give N-(2-(2,6-dioxopiperidin-3-yl)-1-oxoisoindolin-5-yl)-3,3-dimethylindoline-1-carboxamide (56.5 mg, 130 umol, 50% yield) as a white solid.

¹H NMR (400 MHz, DMSO-d₆) δ=10.98 (br s, 1H), 8.86 (s, 1H), 7.93 (s, 1H), 7.87 (d, J=7.9 Hz, 1H), 7.69-7.61 (m, 2H), 7.26-7.22 (m, 1H), 7.20-7.12 (m, 1H), 6.97 (dt, J=0.9, 7.4 Hz, 1H), 5.09 (dd, J=5.1, 13.3 Hz, 1H), 4.47-4.40 (m, 1H), 4.32-4.26 (m, 1H), 3.95 (s, 2H), 2.97-2.86 (m, 1H), 2.65-2.56 (m, 1H), 2.42-2.37 (m, 1H), 2.04-1.95 (m, 1H), 1.34 (s, 6H).

### Example 22. Synthesis of Compound 115

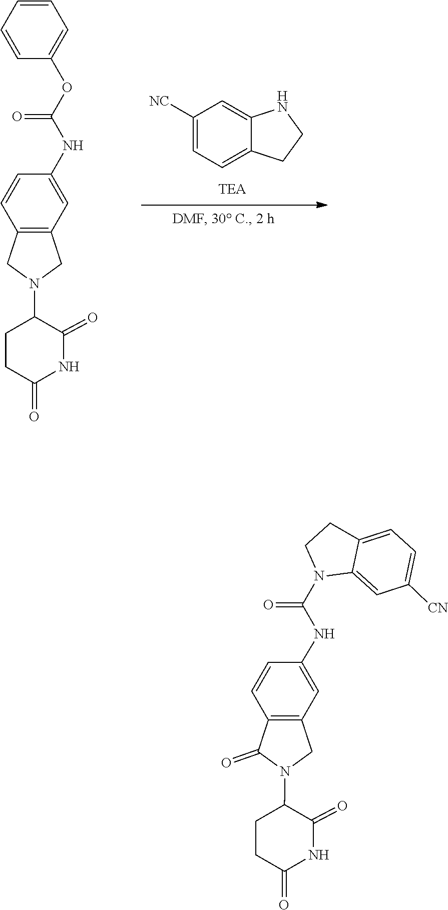 [SMILES: O=C1CCC(N2Cc3ccc(NC(=O)Oc4ccccc4)cc3C2)C(=O)N1 ; N#Cc1ccc2c(c1)NCC2 ; N#Cc1ccc2c(c1)N(C(=O)Nc1ccc3c(c1)CN(C1CCC(=O)NC1=O)C3=O)CC2]

To a mixture of phenyl (2-(2,6-dioxopiperidin-3-yl)-1-oxoisoindolin-5-yl)carbamate (150 mg, 395 umol, 1.10 eq) and indoline-6-carbonitrile (51.8 mg, 359 umol, 1.00 eq) in dimethyformamide (1.00 mL) was added triethylamine (109 mg, 1.08 mmol, 150 μL, 3.00 eq). The mixture was stirred at 30° C. for 2 h. The mixture was quenched by formic acid (0.5 mL) and filtered. The filter cake was added water and lyophilized to give 6-cyano-N-(2-(2,6-dioxopiperidin-3-yl)-1-oxoisoindolin-5-yl)indoline-1-carboxamide (25.71 mg, 59.9 umol, 17% yield) as a grey solid.

¹H NMR (400 MHz, DMSO-d₆) δ=10.98 (s, 1H), 8.99 (s, 1H), 8.14 (s, 1H), 7.93 (s, 1H), 7.67 (s, 2H), 7.45-7.38 (m, 2H), 5.09 (dd, J=5.1, 13.1 Hz, 1H), 4.48-4.41 (m, 1H), 4.34-4.21 (m, 3H), 3.30-3.27 (m, 2H), 2.96-2.87 (m, 1H), 2.62 (br d, J=2.3 Hz, 1H), 2.40 (br dd, J=4.5, 13.0 Hz, 1H), 2.04-1.97 (m, 1H).

### Example 23. Synthesis of Compound 135

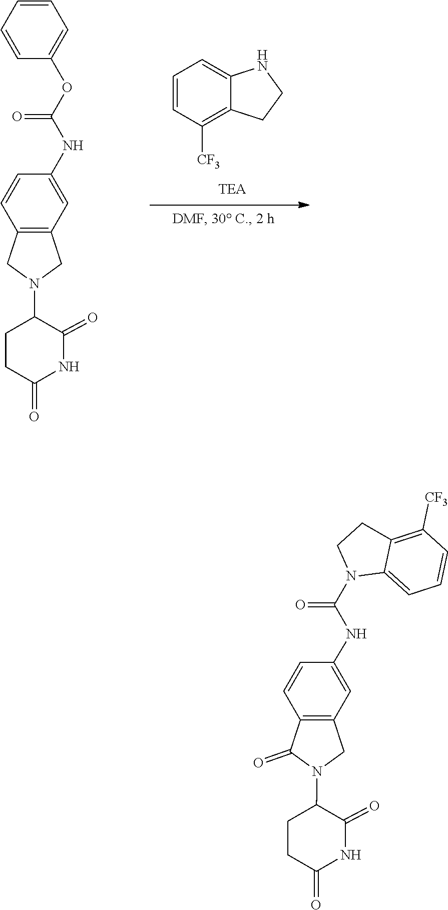 [SMILES: O=C1CCC(N2Cc3ccc(NC(=O)Oc4ccccc4)cc3C2)C(=O)N1 ; FC(F)(F)c1cccc2c1CCN2 ; O=C1CCC(N2Cc3cc(NC(=O)N4CCc5c4cccc5C(F)(F)F)ccc3C2=O)C(=O)N1]

To a mixture of phenyl (2-(2,6-dioxopiperidin-3-yl)-1-oxoisoindolin-5-yl)carbamate (150 mg, 395 umol, 1.10 eq) and 4-(trifluoromethyl)indoline (67.3 mg, 359 umol, 1.00 eq) in dimethyformamide (1.00 mL) was added triethylamine (109 mg, 1.08 mmol, 150 μL, 3.00 eq). The mixture was stirred at 30° C. for 2 h. The mixture was quenched by formic acid (0.5 mL) and filtered. The filter cake was added water and lyophilized to give N-(2-(2,6-dioxopiperidin-3-yl)-1-oxoisoindolin-5-yl)-4-(trifluoromethyl)indoline-1-carboxamide (10.13 mg, 21.4 umol, 6% yield) as a grey solid.

¹H NMR (400 MHz, DMSO-d₆) δ=10.98 (s, 1H), 8.98 (s, 1H), 8.17 (d, J=8.0 Hz, 1H), 7.92 (s, 1H), 7.67 (s, 2H), 7.38 (t, J=7.9 Hz, 1H), 7.24 (d, J=7.8 Hz, 1H), 5.09 (dd, J=5.1, 13.4 Hz, 1H), 4.48-4.39 (m, 1H), 4.33-4.22 (m, 3H), 3.39-3.35 (m, 2H), 2.98-2.86 (m, 1H), 2.62-2.55 (m, 1H), 2.42-2.33 (m, 1H), 2.04-1.95 (m, 1H).

### Example 24. Synthesis of Compound 137

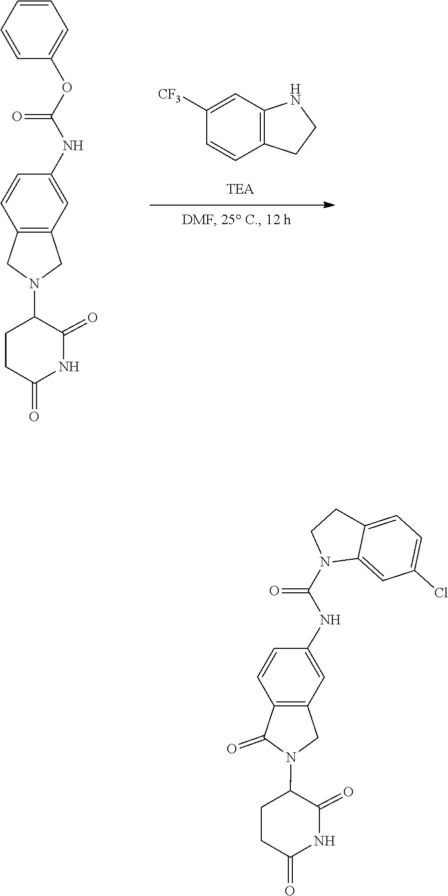 [SMILES: O=C1CCC(N2Cc3ccc(NC(=O)Oc4ccccc4)cc3C2)C(=O)N1 ; FC(F)(F)c1ccc2c(c1)NCC2 ; O=C1CCC(N2Cc3cc(NC(=O)N4CCc5ccc(Cl)cc54)ccc3C2=O)C(=O)N1]

To a solution of phenyl (2-(2,6-dioxopiperidin-3-yl)-1-oxoisoindolin-5-yl)carbamate (300 mg, 790 umol, 1.00 eq) in N,N-dimethyl formamide (3.00 mL) was added 6-chloroindoline (133 mg, 869 umol, 1.10 eq) and triethylamine (240 mg, 2.37 mmol, 330 μL, 3.00 eq). Then the mixture was stirred at 25° C. for 12 h. The mixture was adjusted pH to 5-6 by formic acid (0.2 mL) and it was added dimethylsulfoxide (5 mL). The solution was added into water (20 mL) and filtered. The filter cake was purified by reversed phase column chromatography (C18, 40 g; condition: water/acetonitrile=1/0 to 0/1, 0.1% formic acid) and lyophilized to afford 6-chloro-N-(2-(2,6-dioxopiperidin-3-yl)-1-oxoisoindolin-5-yl)indoline-1-carboxamide (68.6 mg, 153 umol, 27% yield, 98% purity) as a white solid.

¹H NMR (400 MHz, DMSO-d₆) δ=10.99 (s, 1H), 8.93 (s, 1H), 7.96-7.88 (m, 2H), 7.66 (s, 2H), 7.23 (d, J=8.0 Hz, 1H), 6.98 (dd, J=1.9, 7.9 Hz, 1H), 5.10 (dd, J=5.0, 13.3 Hz, 1H), 4.47-4.41 (m, 1H), 4.34-4.27 (m, 1H), 4.26-4.19 (m, 2H), 3.19 (br t, J=8.6 Hz, 2H), 2.98-2.87 (m, 1H), 2.63 (br d, J=2.4 Hz, 1H), 2.40 (br dd, J=4.5, 13.3 Hz, 1H), 2.06-1.96 (m, 1H).

### Example 25. Synthesis of Compound 142

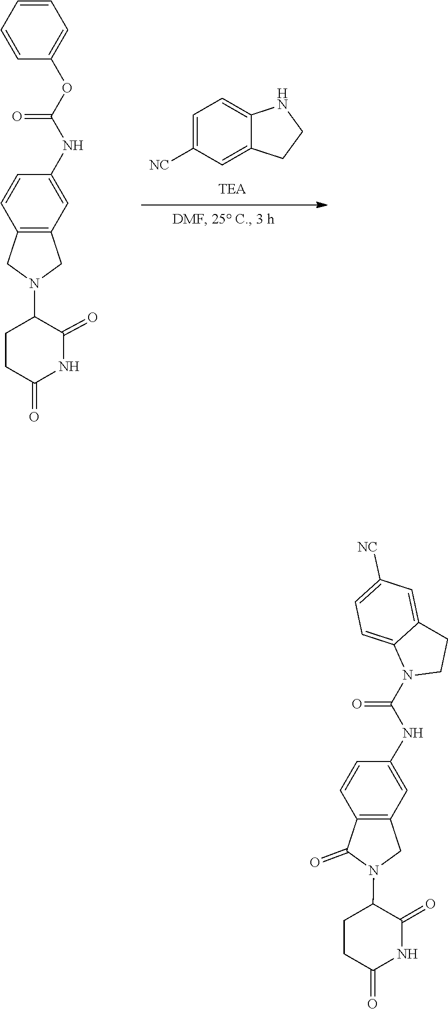 [SMILES: O=C1CCC(N2Cc3ccc(NC(=O)Oc4ccccc4)cc3C2)C(=O)N1 ; N#Cc1ccc2c(c1)CCN2 ; N#Cc1ccc2c(c1)CCN2C(=O)Nc1ccc2c(c1)CN(C1CCC(=O)NC1=O)C2=O]

To a solution of phenyl (2-(2,6-dioxopiperidin-3-yl)-1-oxoisoindolin-5-yl)carbamate (120 mg, 316 umol, 1.00 eq) in dimethyl formamide (1.00 mL) was added triethylamine (96.0 mg, 948 umol, 132 μL, 3.00 eq) and indoline-5-carbonitrile (50.1 mg, 347 umol, 1.10 eq). The mixture was stirred at 25° C. for 3 h. The mixture was filtered to give a filter cake, then it was dried under reduced pressure to give 5-cyano-N-(2-(2,6-dioxopiperidin-3-yl)-1-oxoisoindolin-5-yl)indoline-1-carboxamide (12.0 mg, 26.55 umol, 8% yield, 95% purity) as an off-white solid.

¹H NMR (400 MHz, DMSO-d₆) δ=11.00 (br s, 1H), 9.09 (br s, 1H), 8.17 (s, 1H), 7.99 (br d, J=8.4 Hz, 1H), 7.92 (br s, 1H), 7.68-7.62 (m, 3H), 5.10 (br dd, J=4.8, 13.0 Hz, 1H), 4.51-4.40 (m, 1H), 4.33 (br s, 1H), 4.29-4.23 (m, 2H), 3.24 (br t, J=8.3 Hz, 2H), 2.97-2.88 (m, 1H), 2.60 (br d, J=17.3 Hz, 1H), 2.45-2.35 (m, 1H), 2.07-1.95 (m, 1H).

### Example 26. Synthesis of Compound 163

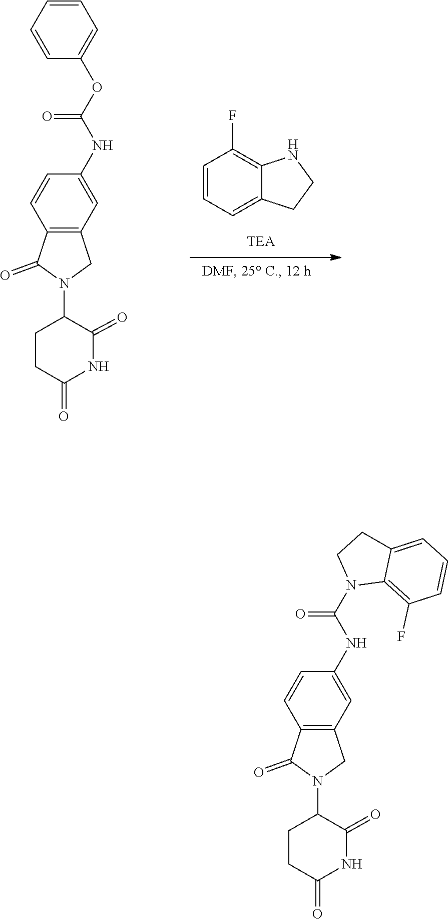 [SMILES: O=C1CCC(N2Cc3cc(NC(=O)Oc4ccccc4)ccc3C2=O)C(=O)N1 ; Fc1cccc2c1NCC2 ; O=C1CCC(N2Cc3cc(NC(=O)N4CCc5cccc(F)c54)ccc3C2=O)C(=O)N1]

To a solution of phenyl (2-(2,6-dioxopiperidin-3-yl)-1-oxoisoindolin-5-yl)carbamate (300 mg, 790 umol, 1.00 eq) in N,N-dimethyl formamide (3.00 mL) was added 7-fluoroindoline (119 mg, 869 umol, 1.10 eq) and triethylamine (240 mg, 2.37 mmol, 330 μL, 3.00 eq). Then the mixture was stirred at 25° C. for 12 h. The mixture was adjust pH to 5-6 by formic acid (0.2 mL) and added dimethylsulfoxide (5 mL). The solution was added water (20 mL) and filtered. The filter cake was purified by purified by reversed phase column chromatography (C18, 40 g; condition: water/acetonitrile=1/0 to 0/1, 0.1% formic acid) and lyophilized to afford N-(2-(2,6-dioxopiperidin-3-yl)-1-oxoisoindolin-5-yl)-7-fluoroindoline-1-carboxamide (10.08 mg, 23.6 umol, 3% yield, 99% purity) as a white solid.

¹H NMR (400 MHz, DMSO-d₆) δ=11.03-10.93 (m, 1H), 9.54 (s, 1H), 7.85 (s, 1H), 7.66-7.62 (m, 1H), 7.61-7.57 (m, 1H), 7.12-7.08 (m, 1H), 7.05-7.00 (m, 2H), 5.08 (dd, J=5.0, 13.4 Hz, 1H), 4.47-4.38 (m, 1H), 4.31-4.24 (m, 1H), 4.14 (t, J=8.1 Hz, 2H), 3.16 (br t, J=8.1 Hz, 2H), 2.96-2.88 (m, 1H), 2.61 (br s, 1H), 2.38 (br dd, J=4.5, 13.0 Hz, 1H), 2.02-1.95 (m, 1H).

### Example 27. Synthesis of Compound 178

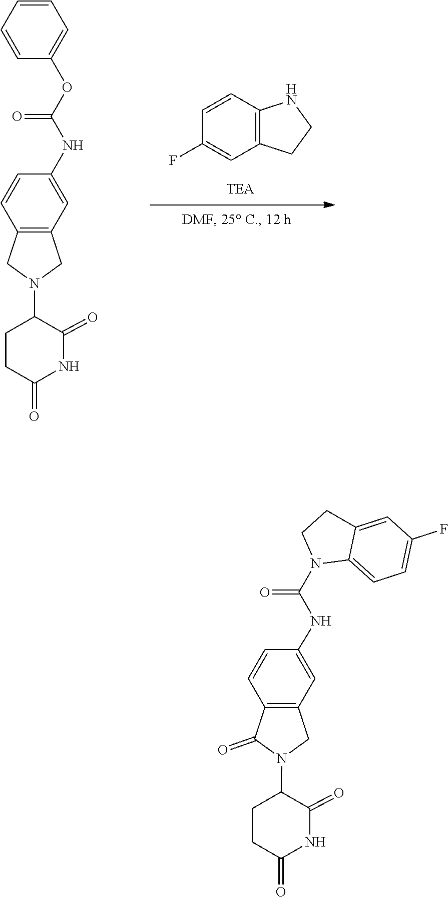 [SMILES: O=C1CCC(N2Cc3ccc(NC(=O)Oc4ccccc4)cc3C2)C(=O)N1 ; Fc1ccc2c(c1)CCN2 ; O=C1CCC(N2Cc3cc(NC(=O)N4CCc5cc(F)ccc54)ccc3C2=O)C(=O)N1]

To a solution of phenyl (2-(2,6-dioxopiperidin-3-yl)-1-oxoisoindolin-5-yl)carbamate (200 mg, 527 umol, 1.10 eq) in N,N-dimethyl formamide (2.00 mL) was added 5-fluoroindoline (83.2 mg, 479 umol, 1.00 eq, hydrochloride) and triethylamine (145 mg, 1.44 mmol, 200 μL, 3.00 eq). Then the mixture was stirred at 25° C. for 12 h. The mixture was adjusted pH to 5-6 by formic acid (0.2 mL) and added dimethylsulfoxide (5 mL). The solution was added water (20 mL) and filtered. The filter cake was purified by reversed phase column chromatography (C18, 40 g; condition: water/acetonitrile=1/0 to 0/1, 0.1% formic acid) and lyophilized to afford N-(2-(2,6-dioxopiperidin-3-yl)-1-oxoisoindolin-5-yl)-5-fluoroindoline-1-carboxamide (55.8 mg, 128 umol, 26% yield, 97% purity) as a white solid.

¹H NMR (400 MHz, DMSO-d₆) δ=10.98 (s, 1H), 8.85 (s, 1H), 7.91 (s, 1H), 7.86 (dd, J=4.9, 8.9 Hz, 1H), 7.65 (d, J=0.9 Hz, 2H), 7.10 (dd, J=2.6, 8.5 Hz, 1H), 6.97 (dt, J=2.7, 9.0 Hz, 1H), 5.10 (dd, J=5.1, 13.3 Hz, 1H), 4.48-4.40 (m, 1H), 4.33-4.26 (m, 1H), 4.20 (t, J=8.7 Hz, 2H), 3.20 (br t, J=8.6 Hz, 2H), 2.97-2.87 (m, 1H), 2.64-2.58 (m, 1H), 2.45-2.37 (m, 1H), 2.05-1.96 (m, 1H).

### Example 28. Synthesis of Compound 185

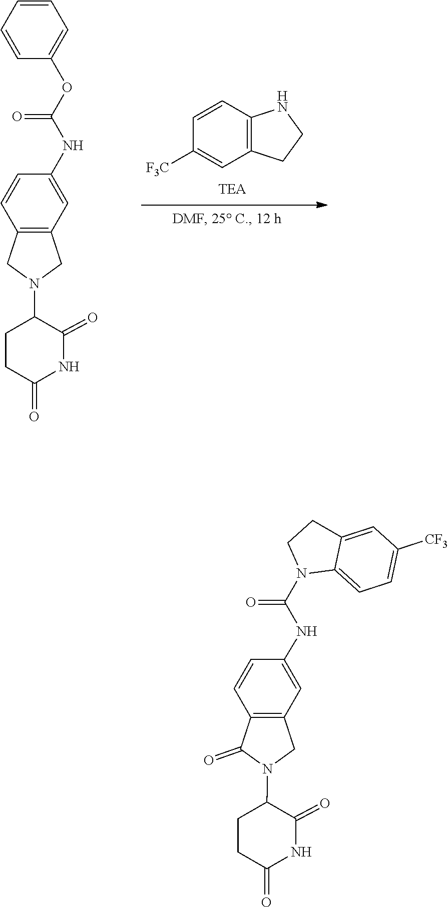 [SMILES: O=C1CCC(N2Cc3ccc(NC(=O)Oc4ccccc4)cc3C2)C(=O)N1 ; FC(F)(F)c1ccc2c(c1)CCN2 ; O=C1CCC(N2Cc3cc(NC(=O)N4CCc5cc(C(F)(F)F)ccc54)ccc3C2=O)C(=O)N1]

To a solution of phenyl (2-(2,6-dioxopiperidin-3-yl)-1-oxoisoindolin-5-yl)carbamate (150 mg, 395 umol, 1.00 eq) in N,N-dimethyl formamide (1.50 mL) was added 5-(trifluoromethyl)indoline (81.4 mg, 434 umol, 1.10 eq) and triethylamine (120 mg, 1.19 mmol, 165 μL, 3.00 eq). Then the mixture was stirred at 25° C. for 12 h. The mixture was adjust pH to 5-6 by formic acid (0.2 mL) and filtered. The filtrate was purified by prep-HPLC (column: Phenomenex luna C18 150*25 mm*10 um; mobile phase: [water (FA)-ACN]; B %: 35%-65%, 9 min) and further purified by prep-HPLC (column: Phenomenex luna C18 150*25 mm*10 um; mobile phase: [water (FA)-ACN]; B %: 36%-66%, 9 min) to afford N-(2-(2,6-dioxopiperidin-3-yl)-1-oxoisoindolin-5-yl)-5-(trifluoromethyl)indoline-1-carboxamide (13.26 mg, 27.8 umol, 6% yield, 99% purity) as a white solid.

¹H NMR (400 MHz, DMSO-d₆) δ=10.99 (s, 1H), 9.00 (s, 1H), 8.03 (d, J=8.4 Hz, 1H), 7.93 (s, 1H), 7.67 (s, 2H), 7.56 (s, 1H), 7.55-7.49 (m, 1H), 5.17-5.05 (m, 1H), 4.49-4.42 (m, 1H), 4.33 (s, 1H), 4.28-4.21 (m, 2H), 2.95-2.87 (m, 1H), 2.63 (br s, 1H), 2.43-2.38 (m, 1H), 2.05-1.96 (m, 1H).

### Example 29. Synthesis of Compound 186

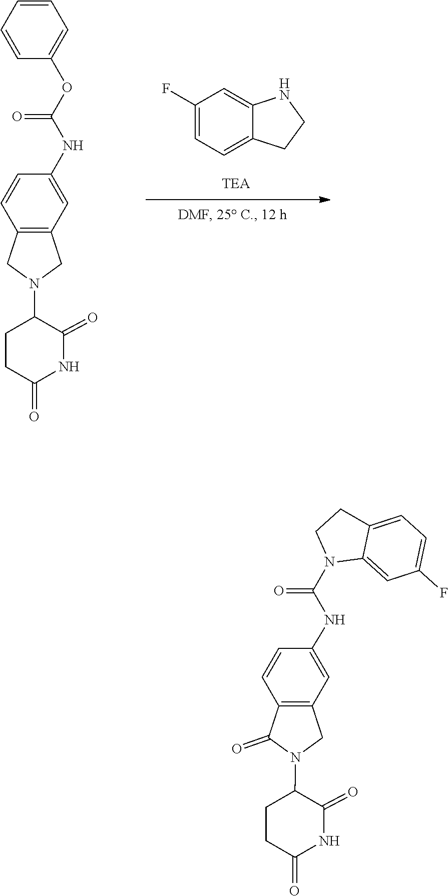 [SMILES: O=C1CCC(N2Cc3ccc(NC(=O)Oc4ccccc4)cc3C2)C(=O)N1 ; Fc1ccc2c(c1)NCC2 ; O=C1CCC(N2Cc3cc(NC(=O)N4CCc5ccc(F)cc54)ccc3C2=O)C(=O)N1]

To a solution of phenyl (2-(2,6-dioxopiperidin-3-yl)-1-oxoisoindolin-5-yl)carbamate (150 mg, 395 umol, 1.00 eq) in N,N-dimethyl formamide (1.50 mL) was added 6-fluoroindoline (59.6 mg, 434 umol, 1.10 eq) and triethylamine (120 mg, 1.19 mmol, 165 μL, 3.00 eq). Then the mixture was stirred at 25° C. for 12 h. The mixture was adjust pH to 5-6 by formic acid (0.2 mL) and filtered. The filtrate was purified by prep-HPLC (column: Phenomenex luna C18 150*25 mm*10 um; mobile phase: [water (FA)-ACN]; B %: 27%-57%, 9 min) and lyophilized to afford N-(2-(2,6-dioxopiperidin-3-yl)-1-oxoisoindolin-5-yl)-6-fluoroindoline-1-carboxamide (10.76 mg, 25.2 umol, 6% yield, 99% purity) as a white solid.

¹H NMR (400 MHz, DMSO-d₆) δ=10.99 (s, 1H), 8.92 (s, 1H), 7.93 (s, 1H), 7.70-7.62 (m, 3H), 7.24-7.16 (m, 1H), 6.74 (dt, J=2.6, 8.7 Hz, 1H), 5.10 (dd, J=5.1, 13.3 Hz, 1H), 4.48-4.41 (m, 1H), 4.35-4.20 (m, 3H), 3.17 (br t, J=8.6 Hz, 2H), 2.98-2.87 (m, 1H), 2.63 (br d, J=2.9 Hz, 1H), 2.40 (br dd, J=4.4, 13.1 Hz, 1H), 2.06-1.97 (m, 1H).

### Example 30. Synthesis of Compound 190

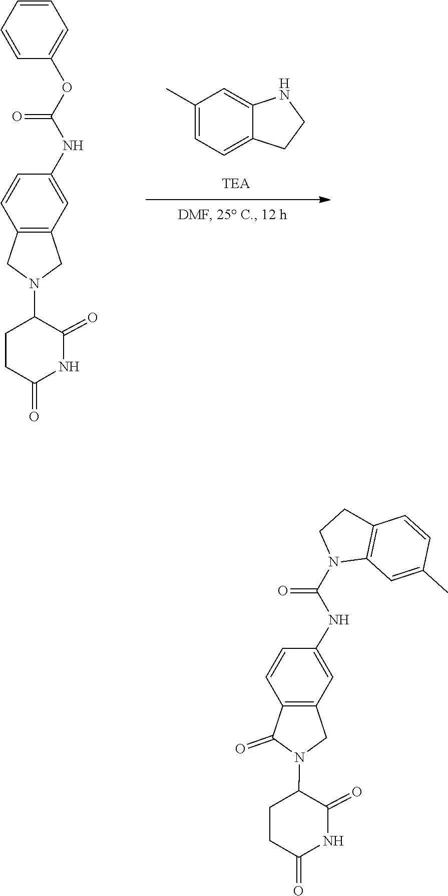

To a solution of phenyl (2-(2,6-dioxopiperidin-3-yl)-1-oxoisoindolin-5-yl)carbamate (150 mg, 395 umol, 1.00 eq), triethylamine (120 mg, 1.19 mmol, 165 μL, 3.00 eq) in dimethyl formamide (2.00 mL) was added 6-methylindoline (57.9 mg, 434 umol, 1.10 eq). The mixture was stirred at 25° C. for 12 h. The reaction mixture was adjusted pH=5 with formic acid (1.00 mL). The mixture was filtered to give a filter cake, which was lyophilized to give N-(2-(2,6-dioxopiperidin-3-yl)-1-oxoisoindolin-5-yl)-6-methylindoline-1-carboxamide (18.3 mg, 43.8 umol, 11% yield) as a gray solid.

¹H NMR (400 MHz, DMSO-d₆) δ=11.13-10.82 (m, 1H), 8.83 (s, 1H), 7.93 (s, 1H), 7.75 (s, 1H), 7.64 (s, 2H), 7.08 (d, J=7.5 Hz, 1H), 6.74 (d, J=7.6 Hz, 1H), 5.09 (dd, J=5.1, 13.3 Hz, 1H), 4.47-4.39 (m, 1H), 4.34-4.24 (m, 1H), 4.16 (t, J=8.6 Hz, 2H), 3.13 (br t, J=8.5 Hz, 2H), 2.98-2.85 (m, 1H), 2.60 (br dd, J=2.0, 15.3 Hz, 1H), 2.46-2.36 (m, 1H), 2.27 (s, 3H), 2.05-1.95 (m, 1H).

### Example 31. Synthesis of Compound 197

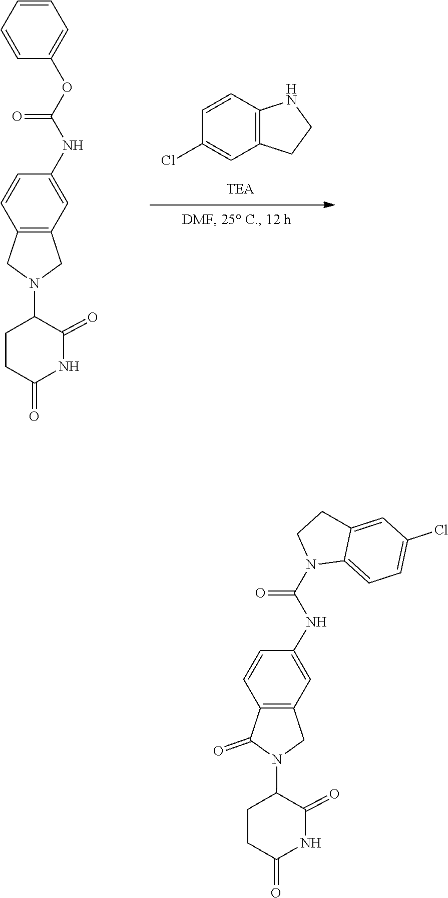 [SMILES: O=C1CCC(N2Cc3ccc(NC(=O)Oc4ccccc4)cc3C2)C(=O)N1 ; Clc1ccc2c(c1)CCN2 ; O=C1CCC(N2Cc3cc(NC(=O)N4CCc5cc(Cl)ccc54)ccc3C2=O)C(=O)N1]

To a solution of phenyl (2-(2,6-dioxopiperidin-3-yl)-1-oxoisoindolin-5-yl)carbamate (150 mg, 395 umol, 1.00 eq) and 5-chloroindoline (60.7 mg, 395 umol, 1.00 eq) in N,N-dimethyl formamide (1.00 mL) was added triethylamine (120 mg, 1.19 mmol, 165 μL, 3.00 eq). Then the mixture was stirred at 25° C. for 12 h. The mixture was filtered. Then the filter cake was dissolved in water and lyophilized to afford 5-chloro-N-(2-(2,6-dioxopiperidin-3-yl)-1-oxoisoindolin-5-yl)indoline-1-carboxamide (90.0 mg, 203 umol, 51% yield, 99% purity) as a black solid.

¹H NMR (400 MHz, DMSO-d₆) δ=10.97 (s, 1H), 8.99 (s, 1H), 7.92 (s, 1H), 7.86 (d, J=8.6 Hz, 1H), 7.71-7.66 (m, 1H), 7.66-7.61 (m, 1H), 7.27 (s, 1H), 7.18 (dd, J=1.9, 8.4 Hz, 1H), 5.09 (dd, J=4.9, 13.2 Hz, 1H), 4.47-4.38 (m, 1H), 4.33-4.26 (m, 1H), 4.22 (t, J=8.8 Hz, 2H), 3.19 (br t, J=8.4 Hz, 2H), 2.96-2.87 (m, 1H), 2.62-2.57 (m, 1H), 2.39 (br dd, J=4.3, 13.1 Hz, 1H), 2.04-1.96 (m, 1H).

### Example 32. Synthesis of Compound 198

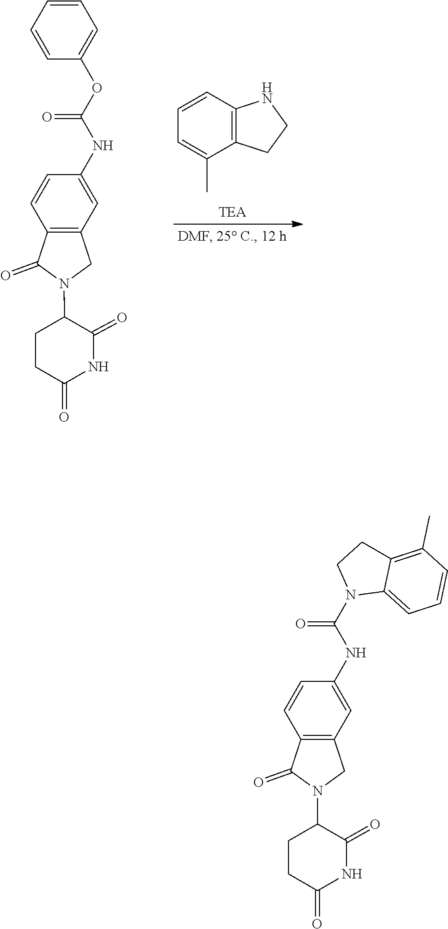

To a solution of phenyl (2-(2,6-dioxopiperidin-3-yl)-1-oxoisoindolin-5-yl)carbamate (150 mg, 395 umol, 1.00 eq) in N,N-dimethyl formamide (1.50 mL) was added 4-methylindoline (57.9 mg, 434 umol, 1.10 eq) and triethylamine (120 mg, 1.19 mmol, 165 μL, 3.00 eq). Then the mixture was stirred at 25° C. for 12 h. The mixture was adjust pH to 5-6 by formic acid (0.2 mL) and filtrate. The filtrate was purified by prep-HPLC (column: Phenomenex luna C18 150*25 mm*10 um; mobile phase: [water (FA)-ACN]; B %: 28%-58%, 9 min) and lyophilized to afford N-(2-(2,6-dioxopiperidin-3-yl)-1-oxoisoindolin-5-yl)-4-methylindoline-1-carboxamide (8.69 mg, 20.3 umol, 5% yield, 98% purity) as a white solid.

¹H NMR (400 MHz, DMSO-d₆) δ=10.99 (s, 1H), 8.82 (s, 1H), 7.93 (s, 1H), 7.72 (d, J=8.1 Hz, 1H), 7.65 (s, 2H), 7.05 (t, J=7.8 Hz, 1H), 6.77 (d, J=7.6 Hz, 1H), 5.10 (dd, J=5.1, 13.3 Hz, 1H), 4.49-4.40 (m, 1H), 4.34-4.26 (m, 1H), 4.23-4.14 (m, 2H), 3.16-3.07 (m, 2H), 2.97-2.87 (m, 1H), 2.63 (br s, 1H), 2.39 (br dd, J=4.4, 13.1 Hz, 1H), 2.21 (s, 3H), 2.05-1.95 (m, 1H).

### Example 33. Synthesis of Compound 201

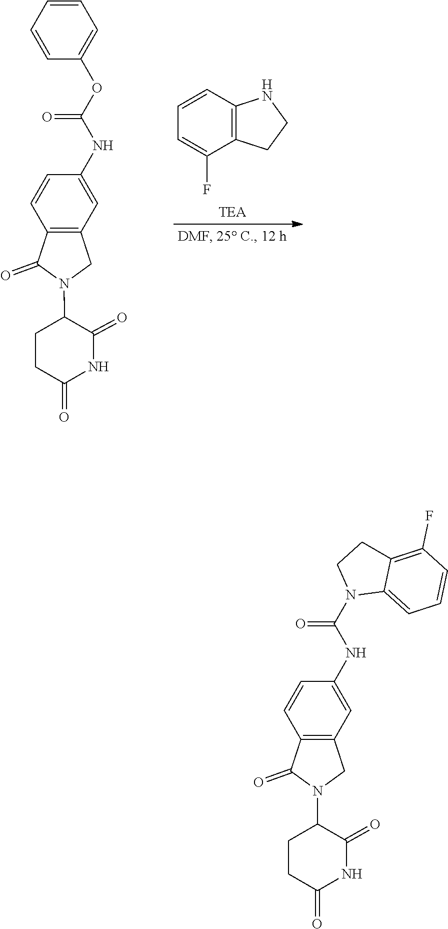 [SMILES: O=C1CCC(N2Cc3cc(NC(=O)Oc4ccccc4)ccc3C2=O)C(=O)N1 ; Fc1cccc2c1CCN2 ; O=C1CCC(N2Cc3cc(NC(=O)N4CCc5c(F)cccc54)ccc3C2=O)C(=O)N1]

To a solution of phenyl (2-(2,6-dioxopiperidin-3-yl)-1-oxoisoindolin-5-yl)carbamate (150 mg, 395 umol, 1.00 eq) and 4-fluoroindoline (54.2 mg, 395 umol, 1.00 eq) in N,N-dimethyl formamide (2.00 mL) was added triethylamine (120 mg, 1.19 mmol, 165 μL, 3.00 eq). The mixture was stirred at 25° C. for 12 h. The mixture was filtered. The filter cake was added water and lyophilized. The crude product was triturated with methyl tert-butyl ether (2 mL) at 25° C. for 30 min. The mixture was filtered, then the filter cake was dried in vacuum to afford N-(2-(2,6-dioxopiperidin-3-yl)-1-oxoisoindolin-5-yl)-4-fluoroindoline-1-carboxamide (11.2 mg, 25.4 umol, 6% yield, 96% purity) as a grey solid.

¹H NMR (400 MHz, DMSO-d₆) δ=10.98 (s, 1H), 8.92 (s, 1H), 7.92 (s, 1H), 7.72 (d, J=8.1 Hz, 1H), 7.66 (s, 2H), 7.25-7.16 (m, 1H), 6.77 (t, J=8.6 Hz, 1H), 5.10 (dd, J=4.9, 13.1 Hz, 1H), 4.49-4.38 (m, 1H), 4.33 (s, 1H), 4.28 (s, 2H), 3.23 (br s, 2H), 2.94-2.88 (m, 1H), 2.61 (br d, J=16.1 Hz, 1H), 2.42-2.38 (m, 1H), 2.04-1.98 (m, 1H).

### Example 34. Synthesis of Compound 214

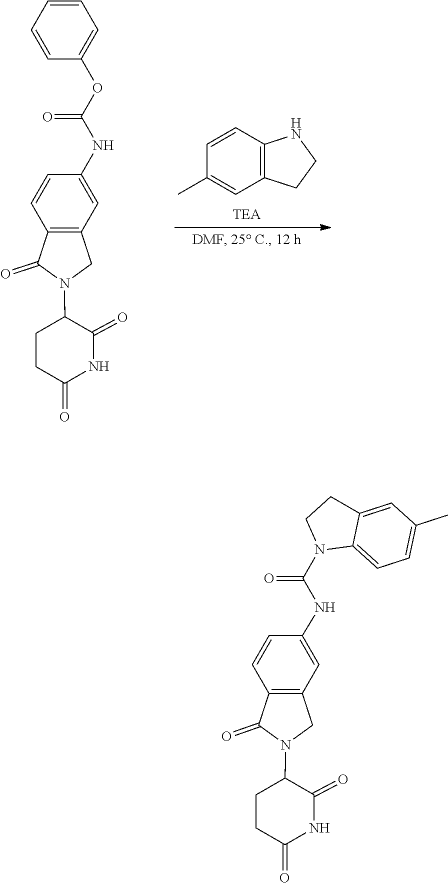

To a solution of phenyl (2-(2,6-dioxopiperidin-3-yl)-1-oxoisoindolin-5-yl)carbamate (150 mg, 395 umol, 1.00 eq) and 5-methylindoline (52.6 mg, 395 umol, 1.00 eq) in N,N-dimethyl formamide (1.00 mL) was added triethylamine (120 mg, 1.19 mmol, 165 μL, 3.00 eq). Then the mixture was stirred at 25° C. for 12 h. The mixture was filtered. Then the filter cake was dissolved in water and lyophilized to afford N-(2-(2,6-dioxopiperidin-3-yl)-1-oxoisoindolin-5-yl)-5-methylindoline-1-carboxamide (12.0 mg, 28.3 umol, 7% yield, 99% purity) as a black solid.

¹H NMR (400 MHz, DMSO-d₆) δ=10.98 (br s, 1H), 8.80 (s, 1H), 7.91 (s, 1H), 7.75 (d, J=8.1 Hz, 1H), 7.64 (s, 2H), 7.03 (s, 1H), 6.94 (br d, J=8.1 Hz, 1H), 5.09 (dd, J=5.0, 13.3 Hz, 1H), 4.48-4.38 (m, 1H), 4.33-4.25 (m, 1H), 4.15 (br t, J=8.6 Hz, 2H), 3.15 (br t, J=8.4 Hz, 2H), 2.97-2.85 (m, 1H), 2.62 (br s, 1H), 2.37 (br s, 1H), 2.24 (s, 3H), 2.05-1.94 (m, 1H).

### Example 35. Synthesis of Compound 291

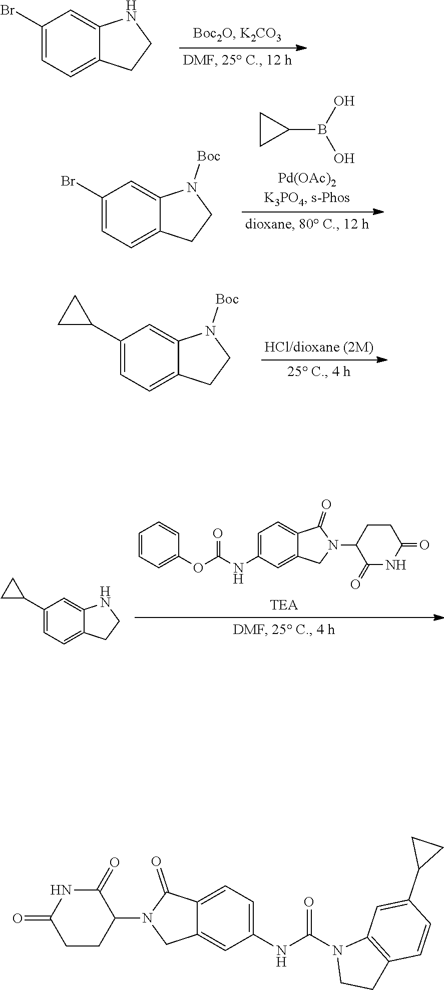

Step 1. To a mixture of 6-bromoindoline (9.50 g, 47.9 mmol, 1.00 eq) in dimethyformamide (95.0 mL) was added potassium carbonate (33.2 g, 239 mmol, 5.00 eq) and di-tert-butyl dicarbonate (10.5 g, 47.9 mmol, 11.0 mL, 1.00 eq). The resulting mixture was stirred at 25° C. for 12 h, and then quenched by addition water (400 mL) and extracted with ethyl acetate (3×400 mL). The combined organic layers were washed with brine (200 mL), dried over sodium sulfate, filtered and concentrated under reduced pressure to give a residue. The residue was purified by column chromatography (SiO₂, Petroleum ether/Ethyl acetate=1/0 to 1/1) and concentrated in vacuum to give tert-butyl 6-bromoindoline-1-carboxylate (11.7 g, 39.2 mmol, 81% yield) as a white solid.

¹H NMR (400 MHz, DMSO-d₆) δ=7.96 (s, 1H), 7.18-7.11 (m, 1H), 7.11-7.04 (m, 1H), 3.91 (t, J=8.8 Hz, 2H), 3.01 (t, J=8.7 Hz, 2H), 1.50 (s, 9H).

Step 2. To a solution of tert-butyl 6-bromoindoline-1-carboxylate (600 mg, 2.01 mmol, 1.00 eq), cyclopropylboronic acid (207 mg, 2.41 mmol, 1.20 eq), potassium phosphate (1.28 g, 6.04 mmol, 3.00 eq) in 1,4-dioxane (5.00 mL) was added dicyclohexyl-[2-(2,6-dimethoxyphenyl)phenyl]phosphane (165 mg, 402 umol, 0.200 eq) and palladium(II) acetate (90.3 mg, 402 umol, 0.200 eq). The mixture was stirred at 80° C. for 12 h under nitrogen. The reaction mixture was diluted with water (40 mL) and then extracted with ethyl acetate (2×80 mL). The organic phase was separated, washed with brine (20 mL), dried over sodium sulfate, filtered and concentrated under reduced pressure to give a residue which was purified by reversed-phase HPLC (column: spherical C18, 20-45 um, 100 Å, SW 120, mobile phase: [water-acetonitrile) to give tert-butyl 6-cyclopropylindoline-1-carboxylate (280 mg, 1.08 mmol, 53% yield) as a yellow solid.

¹H NMR (400 MHz, DMSO-d₆) δ=7.52-7.32 (m, 1H), 7.03 (d, J=7.6 Hz, 1H), 6.67 (br d, J=6.9 Hz, 1H), 3.87 (t, J=8.6 Hz, 2H), 3.01-2.94 (m, 2H), 1.92-1.82 (m, 1H), 1.50 (s, 9H), 0.93-0.87 (m, 2H), 0.61-0.54 (m, 2H).

Step 3. A solution of tert-butyl 6-cyclopropylindoline-1-carboxylate (270 mg, 1.04 mmol, 1.00 eq) in dioxane/hydrochloride (4.00 mL) was stirred at 25° C. for 4 h. The reaction mixture was concentrated under reduced pressure to give 6-cyclopropylindoline (300 mg, crude) as a yellow solid. ¹H NMR (400 MHz, DMSO-d₆) δ=7.32 (d, J=7.6 Hz, 1H), 7.13-7.09 (m, 2H), 3.68 (t, J=7.8 Hz, 2H), 3.59-3.54 (m, 1H), 3.13 (t, J=7.8 Hz, 2H), 2.05-1.96 (m, 1H), 1.02-0.96 (m, 2H), 0.69-0.63 (m, 2H).

Step 4. To a solution of phenyl (2-(2,6-dioxopiperidin-3-yl)-1-oxoisoindolin-5-yl)carbamate (200 mg, 527 umol, 1.00 eq) in dimethyl formamide (3.00 mL) was added triethylamine (160 mg, 1.58 mmol, 220 μL, 3.00 eq), 6-cyclopropylindoline (92.3 mg, 579 umol, 1.10 eq). The resulting mixture was stirred at 25° C. for 4 h, and then filtered to give a filter liquor which was purified by reversed-phase HPLC (column: spherical C18, 20-45 um, 100 Å, SW 80, mobile phase: [water (0.1% Formic Acid)-acetonitrile) to give 6-cyclopropyl-N-(2-(2,6-dioxopiperidin-3-yl)-1-oxoisoindolin-5-yl)indoline-1-carboxamide (75.0 mg, 168 umol, 32% yield) as a white solid.

¹H NMR (400 MHz, DMSO-d₆) δ=10.97 (br s, 1H), 8.81 (s, 1H), 7.93 (s, 1H), 7.68-7.60 (m, 3H), 7.12-7.04 (m, 1H), 6.68 (d, J=8.4 Hz, 1H), 5.09 (dd, J=5.1, 13.3 Hz, 1H), 4.47-4.39 (m, 1H), 4.33-4.27 (m, 1H), 4.23-4.11 (m, 2H), 3.18-3.09 (m, 2H), 2.97-2.87 (m, 1H), 2.60 (br d, J=16.3 Hz, 1H), 2.46-2.36 (m, 1H), 2.05-1.96 (m, 1H), 1.93-1.84 (m, 1H), 0.97-0.87 (m, 2H), 0.63-0.56 (m, 2H).

### Example 36. Synthesis of Compound 292

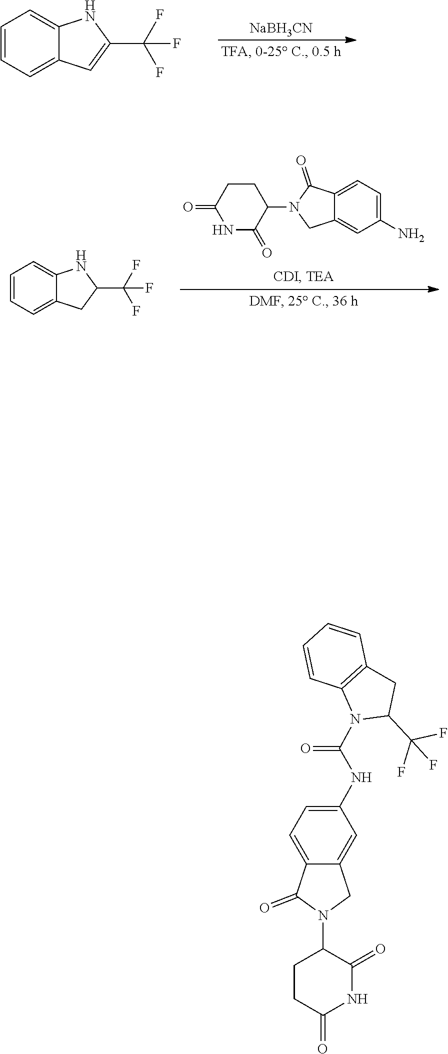 [SMILES: FC(F)(F)c1cc2ccccc2[nH]1 ; "NaBH3CN" ; Nc1ccc2c(c1)CN(C1CCC(=O)NC1=O)C2=O ; FC(F)(F)C1Cc2ccccc2N1 ; O=C1CCC(N2Cc3cc(NC(=O)N4c5ccccc5CC4C(F)(F)F)ccc3C2=O)C(=O)N1]

Step 1. To a solution of 2-(trifluoromethyl)-1H-indole (920 mg, 4.97 mmol, 1.00 eq) in trifluoroacetic acid (10.0 mL) was added sodium cyanoborohydride (468 mg, 7.45 mmol, 1.50 eq) at 0° C. The resulting mixture was stirred at 25° C. for 0.5 h, and then the mixture was adjusted pH=8 with saturated sodium bicarbonate solution. The reaction mixture was diluted with water (20.0 mL) and extracted with ethyl acetate (20.0 mL). The organic phase was separated, washed with brine (10.0 mL), dried over anhydrous sodium sulfate, filtered and concentrated under reduced pressure to give a residue. The residue was purified by reversed-phase HPLC (column: spherical C18, 20-45 um, 100 Å, SW 40, mobile phase: [water (0.1% Formic Acid)-ACN]; B %: 5%-45%, 30 min). The desired fraction was collected and concentrated reduced pressure to afford 2-(trifluoromethyl)indoline (400 mg, 2.14 mmol, 43% yield) as a white solid.

¹H NMR (400 MHz, DMSO-d₆) δ=7.05 (d, J=7.3 Hz, 1H), 6.97 (t, J=7.6 Hz, 1H), 6.61 (t, J=7.4 Hz, 1H), 6.56 (d, J=7.8 Hz, 1H), 6.33 (br d, J=3.5 Hz, 1H), 4.53-4.40 (m, 1H), 3.31 (br s, 1H), 2.99 (dd, J=7.0, 16.5 Hz, 1H)

Step 2. To a solution of 2-(trifluoromethyl)indoline (20.0 mg, 106 umol, 1.00 eq) and 3-(5-amino-1-oxoisoindolin-2-yl)piperidine-2,6-dione (28.8 mg, 111 umol, 1.04 eq) in dimethyl formamide (1.00 mL) was added di(1H-imidazol-1-yl)methanone (25.9 mg, 160 umol, 1.50 eq) and triethylamine (32.4 mg, 320 umol, 44.6 uL, 3.00 eq). The mixture was stirred at 25° C. for 12 h. The resulting mixture was added with 2-(trifluoromethyl)indoline (50.0 mg, 267 umol, 2.50 eq), 3-(5-amino-1-oxoisoindolin-2-yl)piperidine-2,6-dione (60.0 mg, 231 umol, 2.17 eq) and 1,1′-carbonyldiimidazole (250 mg, 1.54 mmol, 14.4 eq), and then stirred for another 24 h. The mixture was filtered, and the filtrate was purified by Prep-HPLC (column: Phenomenex luna C18 150*25 mm*10 um; mobile phase: [water (FA)-ACN]; B %: 28%-58%, 10 min). The desired fraction was collected and lyophilized to afford N-(2-(2,6-dioxopiperidin-3-yl)-1-oxoisoindolin-5-yl)-2-(trifluoromethyl)indoline-1-carboxamide (11.6 mg, 24.5 umol, 23% yield, 97% purity) as a white solid.

¹H NMR (400 MHz, DMSO-d₆) δ=10.98 (s, 1H), 9.33 (s, 1H), 7.87 (s, 1H), 7.83 (d, J=8.1 Hz, 1H), 7.71-7.65 (m, 1H), 7.65-7.58 (m, 1H), 7.29 (d, J=7.3 Hz, 1H), 7.21 (t, J=7.9 Hz, 1H), 7.06-6.99 (m, 1H), 5.80-5.68 (m, 1H), 5.10 (dd, J=5.1, 13.2 Hz, 1H), 4.50-4.41 (m, 1H), 4.36-4.27 (m, 1H), 3.67 (dd, J=10.1, 16.8 Hz, 1H), 3.17 (br d, J=16.6 Hz, 1H), 2.98-2.86 (m, 1H), 2.64-2.56 (m, 1H), 2.45-2.35 (m, 1H), 2.05-1.95 (m, 1H).

### Example 37. Synthesis of Compound 293

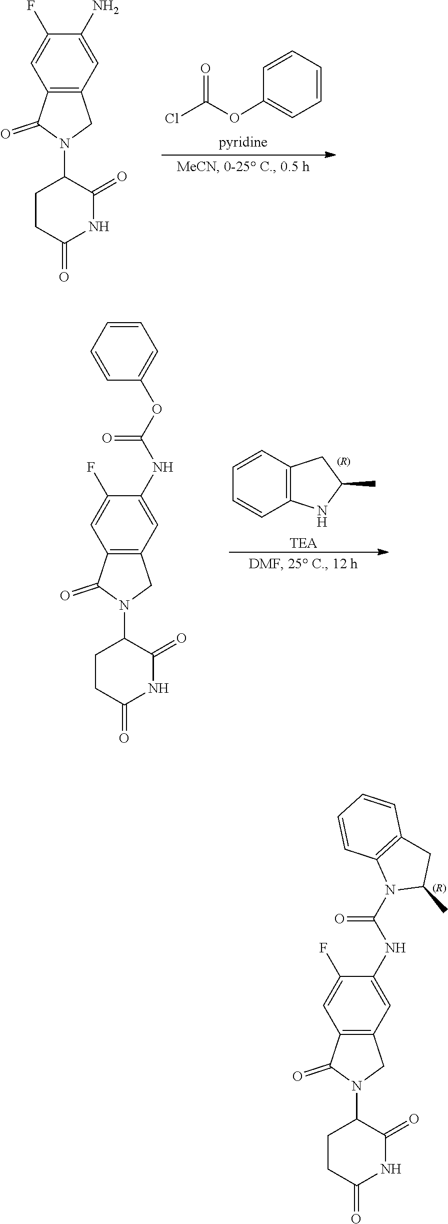

Step 1. To a solution of 3-(5-amino-6-fluoro-1-oxoisoindolin-2-yl)piperidine-2,6-dione (180 mg, 649 umol, 1.00 eq) in acetonitrile (10.0 mL) was added pyridine (154 mg, 1.95 mmol, 157 uL, 3.00 eq). Phenyl carbonochloridate (121 mg, 779 umol, 97.5 uL, 1.20 eq) was added into the resulting mixture at 0° C., and then the mixture stirred at 0° C. for 0.5 h. The mixture was filtered. The filtrate was purified by reversed-phase HPLC (column: spherical C18, 20-45 um, 100 Å, SW 40, mobile phase: [water (0.1% Formic Acid)-ACN]; B %: 5%-45%, 30 min). The desired fraction was collected and lyophilized to afford phenyl (2-(2,6-dioxopiperidin-3-yl)-6-fluoro-1-oxoisoindolin-5-yl)carbamate (70.0 mg, 176 umol, 27% yield) as a white solid.

¹H NMR (400 MHz, DMSO-d₆) δ=11.05-10.75 (m, 1H), 9.41-9.15 (m, 1H), 7.27 (d, J=10.4 Hz, 1H), 7.19-7.08 (m, 2H), 6.85 (d, J=7.8 Hz, 1H), 6.73 (s, 1H), 5.88 (s, 2H), 5.02 (dd, J=5.1, 13.4 Hz, 1H), 4.30-4.22 (m, 1H), 4.17-4.07 (m, 1H), 2.94-2.82 (m, 1H), 2.57 (td, J=2.0, 15.4 Hz, 1H), 2.32 (br dd, J=4.4, 13.1 Hz, 1H), 1.99-1.90 (m, 1H)

Step 2. To a solution of phenyl (2-(2,6-dioxopiperidin-3-yl)-6-fluoro-1-oxoisoindolin-5-yl)carbamate (70.0 mg, 176 umol, 1.00 eq) in N,N-dimethyl formamide (1.00 mL) was added triethylamine (26.7 mg, 264 umol, 36.7 uL, 1.50 eq) and (R)-2-methylindoline (23.4 mg, 176 umol, 1.00 eq). The resulting mixture was stirred at 25° C. for 12 h. The mixture was filtered. The filtrate was purified by Prep-HPLC (column: Phenomenex luna C18 150*25 mm*10 um; mobile phase: [water (FA)-ACN]; B %: 31%-61%, 10 min). The desired fraction was collected and lyophilized to afford (2R)—N-(2-(2,6-dioxopiperidin-3-yl)-6-fluoro-1-oxoisoindolin-5-yl)-2-methylindoline-1-carboxamide (31.2 mg, 67.3 umol, 38% yield, 98% purity) as a white solid.

¹H NMR (400 MHz, DMSO-d₆) δ=11.11-10.87 (m, 1H), 8.69 (br s, 1H), 8.00-7.72 (m, 2H), 7.69-7.49 (m, 1H), 7.24 (br d, J=5.6 Hz, 1H), 7.18-7.09 (m, 1H), 6.95 (br t, J=7.1 Hz, 1H), 5.11 (br d, J=13.4 Hz, 1H), 4.93-4.77 (m, 1H), 4.51-4.37 (m, 1H), 4.36-4.24 (m, 1H), 3.41 (br d, J=6.5 Hz, 2H), 2.99-2.84 (m, 1H), 2.66 (br dd, J=1.6, 6.2 Hz, 1H), 2.41 (br s, 1H), 2.09-1.95 (m, 1H), 1.32-1.20 (m, 3H).

### Example 38. Synthesis of Compound 205

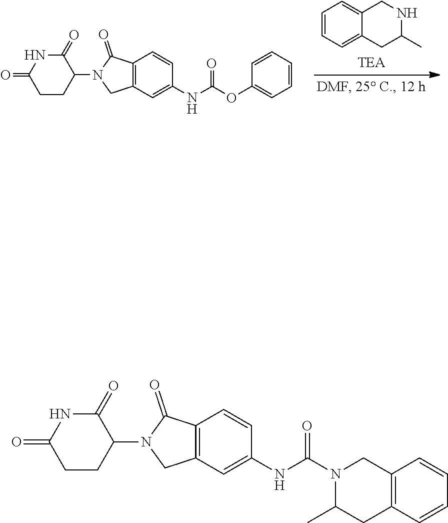 [SMILES: O=C1CCC(N2Cc3cc(NC(=O)Oc4ccccc4)ccc3C2=O)C(=O)N1 ; CC1Cc2ccccc2CN1 ; CC1Cc2ccccc2CN1C(=O)Nc1ccc2c(c1)CN(C1CCC(=O)NC1=O)C2=O]

To a solution of phenyl (2-(2,6-dioxopiperidin-3-yl)-1-oxoisoindolin-5-yl)carbamate (150 mg, 395 umol, 1.00 eq) and 3-methyl-1,2,3,4-tetrahydroisoquinoline (58.2 mg, 395 umol, 1.00 eq) in N,N-dimethyl formamide (2.00 mL) was added triethylamine (120 mg, 1.19 mmol, 165 uL, 3.00 eq). The mixture was stirred at 25° C. for 12 h. The mixture was filtered. The filter liquor was purified by prep-HPLC (column: Phenomenex luna C18 150*25 mm*10 um; mobile phase: [water (FA)-ACN]; B %: 28%-58%, 9 min). The desired fraction was collected and lyophilized to afford N-(2-(2,6-dioxopiperidin-3-yl)-1-oxoisoindolin-5-yl)-3-methyl-3,4-dihydroisoquinoline-2(1H)-carboxamide (41.28 mg, 94.5 umol, 23% yield, 99% purity) as an off-white solid.

¹H NMR (400 MHz, DMSO-d₆) δ=10.97 (s, 1H), 8.85 (s, 1H), 7.82 (s, 1H), 7.61-7.55 (m, 2H), 7.21 (s, 4H), 5.07 (dd, J=5.0, 13.3 Hz, 1H), 4.89 (d, J=16.6 Hz, 1H), 4.77-4.67 (m, 1H), 4.39 (br d, J=15.0 Hz, 2H), 4.31-4.23 (m, 1H), 3.08 (br dd, J=5.6, 15.8 Hz, 1H), 2.96-2.86 (m, 1H), 2.67 (dd, J=1.9, 15.8 Hz, 1H), 2.62-2.56 (m, 1H), 2.40-2.31 (m, 1H), 2.03-1.95 (m, 1H), 1.05 (d, J=6.5 Hz, 3H).

### Example 39. Synthesis of Compound 123

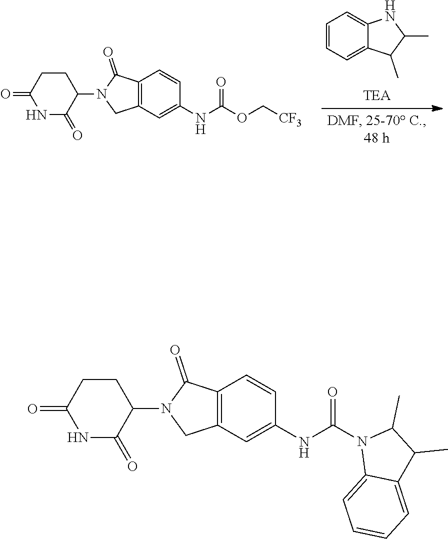 [SMILES: O=C1CCC(N2Cc3cc(NC(=O)OCC(F)(F)F)ccc3C2=O)C(=O)N1 ; CC1Nc2ccccc2C1C ; CC1c2ccccc2N(C(=O)Nc2ccc3c(c2)CN(C2CCC(=O)NC2=O)C3=O)C1C]

To a solution of 2,2,2-trifluoroethyl (2-(2,6-dioxopiperidin-3-yl)-1-oxoisoindolin-5-yl)carbamate (100 mg, 259 umol, 1.00 eq), 2,3-dimethylindoline (49.6 mg, 337 umol, 1.30 eq) in dimethyl formamide (3.00 mL) was added triethylamine (78.7 mg, 778 umol, 108 μL, 3.00 eq) at 25° C. The mixture was stirred at 70° C. for 48 h. The mixture was filtered. The filter liquor was purified by Prep-HPLC (column: Welch Ultimate XB-Diol 250*50*10 um; mobile phase: [Hexane-ethyl alcohol]; B %: 10%-48%, 18 min) and triturated with ethyl acetate (10 mL) and filtered. The filter cake was washed with ethyl acetate (5 mL) and dried to give N-(2-(2,6-dioxopiperidin-3-yl)-1-oxoisoindolin-5-yl)-2,3-dimethylindoline-1-carboxamide (30.0 mg, 69.3 umol, 26% yield) a white solid.

¹H NMR (400 MHz, DMSO-d₆) δ=10.98 (br s, 1H), 8.99 (s, 1H), 7.91 (s, 1H), 7.84 (d, J=8.0 Hz, 1H), 7.65 (s, 2H), 7.27-7.20 (m, 1H), 7.16 (br t, J=7.8 Hz, 1H), 6.97 (q, J=7.5 Hz, 1H), 5.09 (dd, J=4.9, 13.3 Hz, 1H), 4.50-4.38 (m, 2H), 4.34-4.25 (m, 1H), 2.99-2.86 (m, 2H), 2.63-2.57 (m, 1H), 2.45-2.36 (m, 1H), 2.04-1.96 (m, 1H), 1.32-1.21 (m, 5H), 1.06 (d, J=6.3 Hz, 1H).

### Example 40. Synthesis of Compound 187

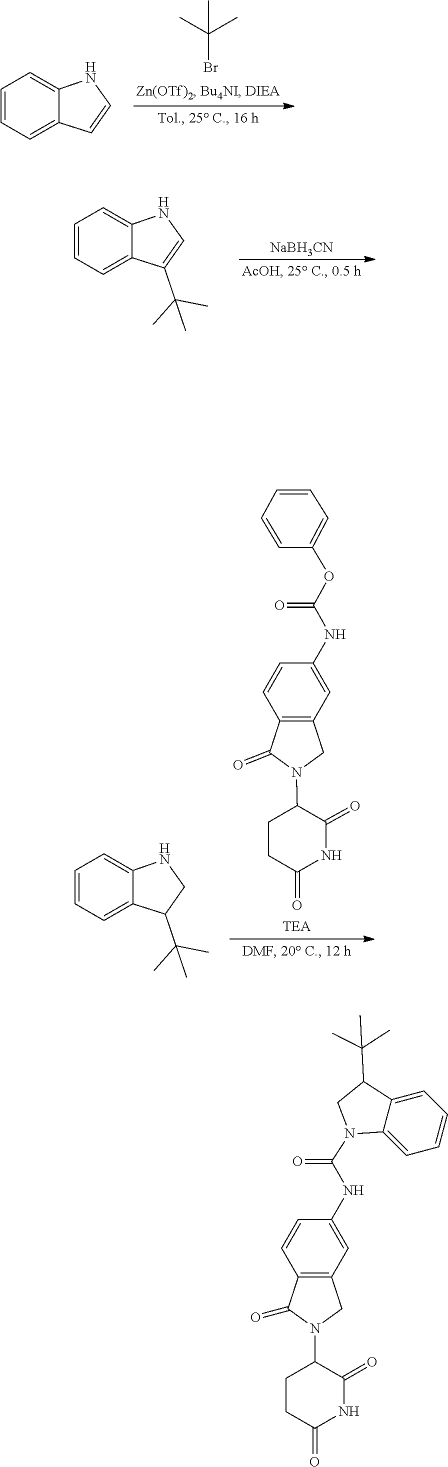

Step 1. To a solution of 2-bromo-2-methyl-propane (600 mg, 4.38 mmol, 508 uL, 1.00 eq), tetrabutylammonium iodide (1.62 g, 4.38 mmol, 1.00 eq), zinctrifluoromethanesulfonate (2.00 g, 5.25 mmol, 1.20 eq) in anhydrous toluene (60.0 mL) was added N,N-diisopropylethylamine (1.25 g, 9.63 mmol, 1.68 mL, 2.20 eq) under nitrogen atmosphere. The mixture was stirred at 25° C. for 15 min, followed by addition of indole (1.03 g, 8.76 mmol, 2.00 eq). The mixture was stirred at 25° C. for 16 h. The mixture was filtered to give the filtrate and concentrated under reduced pressure. The mixture was purified by reversed-phase HPLC (column: spherical C 18, 20-45 um, 100 Å, SW 120, mobile phase: [water (0.1% Formic Acid)-acetonitrile) and extracted with ethyl acetate (3×30 mL). The combined organic layers were washed with brine (30 mL), dried over anhydrous sodium sulfate, filtered and concentrated under reduced pressure to afford 3-(tert-butyl)-1H-indole (800 mg, crude) as yellow oil. ¹H NMR (400 MHz, DMSO-d₆) δ=10.71 (br s, 1H), 7.69 (d, J=8.0 Hz, 1H), 7.34 (d, J=8.0 Hz, 1H), 7.06-7.02 (m, 1H), 7.01 (d, J=2.4 Hz, 1H), 6.98-6.91 (m, 1H), 1.39 (s, 9H).

Step 2. To a solution of 3-(tert-butyl)-1H-indole (200 mg, 1.15 mmol, 1.00 eq) in acetic acid (5.00 mL) was added sodium cyanoborohydride (109 mg, 1.73 mmol, 1.50 eq) at 0° C. The mixture was stirred at 25° C. for 0.5 h. The mixture was adjusted to pH=6-7 with saturated sodium bicarbonate aqueous solution and extracted with ethyl acetate (3×20.0 mL). The combined organic layers were washed with brine (20.0 mL), dried over anhydrous sodium sulfate, filtered and concentrated under reduced pressure to afford 3-(tert-butyl)indoline (200 mg, crude) as yellow oil. MS (ESI) m/z 176.3 [M+H]⁺

Step 3. To a solution of phenyl (2-(2,6-dioxopiperidin-3-yl)-1-oxoisoindolin-5-yl)carbamate (80.0 mg, 211 umol, 1.00 eq), 3-(tert-butyl)indoline (55.4 mg, 316 umol, 1.50 eq) in N,N-dimethyl formamide (1.00 mL) was added triethylamine (64.0 mg, 633 umol, 88.1 uL, 3.00 eq). The mixture was stirred at 25° C. for 12 h. The mixture was filtered. The filtrate was purified by Prep-HPLC (column: Phenomenex luna C18 150*25 mm*10 um; mobile phase: [water (formic acid)-acetonitrile]; B %: 41%-71%, 9 min) to afford 3-(tert-butyl)-N-(2-(2,6-dioxopiperidin-3-yl)-1-oxoisoindolin-5-yl)indoline-1-carboxamide (20.4 mg, 43.5 umol, 21% yield, 98% purity) as an off-white solid.

¹H NMR (400 MHz, DMSO-d₆) δ=10.97 (br s, 1H), 8.88 (s, 1H), 7.99-7.85 (m, 2H), 7.66 (s, 2H), 7.28 (d, J=7.4 Hz, 1H), 7.18 (t, J=7.7 Hz, 1H), 6.97-6.91 (t, J=7.2 Hz, 1H), 5.09 (dd, J=5.1, 13.4 Hz, 1H), 4.47-4.39 (m, 1H), 4.33-4.26 (m, 1H), 4.20 (dd, J=3.3, 10.9 Hz, 1H), 4.05 (t, J=10.1 Hz, 1H), 3.14 (br dd, J=2.8, 9.5 Hz, 1H), 2.97-2.86 (m, 1H), 2.60 (br d, J=16.9 Hz, 1H), 2.39 (br dd, J=4.3, 13.0 Hz, 1H), 2.04-1.96 (m, 1H), 0.93 (s, 9H).

### Example 41. Synthesis of Compound 203

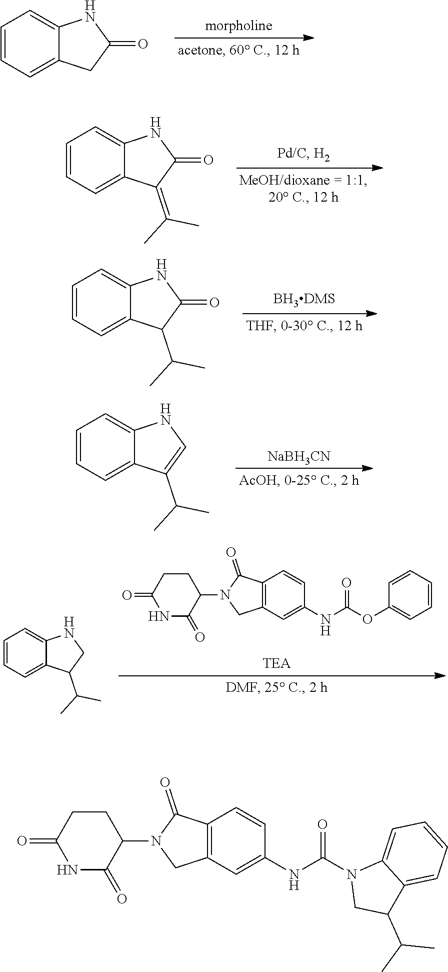

Step 1. To a solution of indolin-2-one (500 mg, 3.76 mmol, 1.00 eq) in acetone (15.0 mL) was added morpholine (163 mg, 1.88 mmol, 165 uL, 0.500 eq). Then the mixture was stirred at 60° C. for 12 h. The reaction mixture was diluted with water (50 mL) and extracted with ethyl acetate (3×30 mL). The combined organic layers were washed with brine, dried over anhydrous sodium sulfate, filtered and concentrated under reduced pressure to give a residue. The residue was purified by column chromatography (SiO₂, petroleum ether/ethyl acetate=50/1 to 10/1) and concentrated in vacuum to afford 3-isopropylideneindolin-2-one (600 mg, 3.36 mmol, 89% yield, 97% purity) as a yellow solid.

¹H NMR (400 MHz, DMSO-d₆) δ=10.40 (br s, 1H), 7.52 (d, J=7.6 Hz, 1H), 7.20-7.14 (m, 1H), 6.95 (dt, J=0.9, 7.7 Hz, 1H), 6.81 (d, J=7.6 Hz, 1H), 2.50 (d, J=1.8 Hz, 3H), 2.32 (s, 3H).

Step 2. To a solution of 3-isopropylideneindolin-2-one (500 mg, 2.89 mmol, 1.00 eq) in methanol (15.0 mL) and dichloromethane (15.0 mL) was added palladium on activated carbon (250 mg, 10% purity). Then the mixture was stirred at 20° C. for 12 h under hydrogen atmosphere (15 Psi). The mixture was filtered. The filtrate was concentrated in vacuum to afford 3-isopropylindolin-2-one (500 mg, crude) as a white solid.

¹H NMR (400 MHz, CDCl₃) δ=7.76 (br s, 1H), 7.14 (t, J=7.7 Hz, 1H), 7.01-6.91 (m, 1H), 6.79 (d, J=7.8 Hz, 1H), 3.32 (d, J=3.5 Hz, 1H), 2.43 (dtd, J=3.6, 6.9, 13.9 Hz, 1H), 1.53 (br s, 2H), 1.05 (d, J=7.0 Hz, 3H), 0.86 (d, J=6.8 Hz, 3H).

Step 3. To a solution of 3-isopropylindolin-2-one (500 mg, 2.85 mmol, 1.00 eq) in tetrahydrofuran (15.0 mL) was added borane dimethyl sulfide complex (10.0 M, 1.14 mL, 4.00 eq). Then the mixture was stirred at 30° C. for 12 h under nitrogen atmosphere. The mixture was added in methanol (10 mL). The mixture was concentrated in vacuum. The residue was diluted with water (50 mL) and extracted with dichloromethane (3×50 mL). The combined organic layers were washed with brine (3×10 mL), dried over anhydrous sodium sulfate, filtered and concentrated under reduced pressure to give a residue. The residue was purified by reversed phase column chromatography (C18, 40 g; condition: water/acetonitrile=1/0 to 0/1, 0.1% formic acid) and concentrated in vacuum. The mixture was extracted with dichloromethane (3×50 mL). The combined organic layers were washed with brine (3×10 mL), dried over anhydrous sodium sulfate, filtered and concentrated under reduced pressure to give 3-isopropyl-1H-indole3-isopropyl-1H-indole (220 mg, 1.37 mmol, 47% yield, 99% purity) as a yellow oil.

¹H NMR (400 MHz, CDCl₃) δ=7.88-7.74 (m, 1H), 7.59 (d, J=8.0 Hz, 1H), 7.28 (d, J=8.1 Hz, 1H), 7.11 (t, J=7.4 Hz, 1H), 7.07-7.01 (m, 1H), 6.89 (d, J=2.1 Hz, 1H), 3.15 (td, J=6.8, 13.7 Hz, 1H), 1.30 (d, J=6.9 Hz, 6H).

Step 4. To a solution of 3-isopropyl-1H-indole3-isopropyl-1H-indole (220 mg, 1.38 mmol, 1.00 eq) in acetic acid (4.00 mL) was added sodium cyanoborohydride (173 mg, 2.76 mmol, 2.00 eq) at 0° C. Then the mixture was stirred at 25° C. for 2 h. The pH of the reaction mixture was adjusted to 7-8 by saturated sodium bicarbonate solution (1M, 100 mL) and extracted with ethyl acetate (3×50 mL). The combined organic layers were washed with brine (3×20 mL), dried over anhydrous sodium sulfate, filtered and concentrated under reduced pressure to give 3-isopropylindoline (140 mg, 868 umol, 62% yield) as yellow oil.

¹H NMR (400 MHz, CDCl₃) δ=7.03 (d, J=7.4 Hz, 1H), 6.95 (t, J=7.6 Hz, 1H), 6.63 (dt, J=0.8, 7.4 Hz, 1H), 6.55 (d, J=7.8 Hz, 1H), 3.54-3.46 (m, 1H), 3.30 (dd, J=6.3, 9.2 Hz, 1H), 3.15 (td, J=5.6, 9.7 Hz, 1H), 1.99-1.94 (m, 1H), 0.92 (d, J=6.9 Hz, 3H), 0.82 (d, J=6.9 Hz, 3H).

Step 5. To a solution of 3-isopropylindoline (57.9 mg, 359 umol, 1.00 eq) and phenyl (2-(2,6-dioxopiperidin-3-yl)-1-oxoisoindolin-5-yl)carbamate (150 mg, 395 umol, 1.10 eq) in N,N-dimethyl formamide (0.500 mL) was added triethylamine (109 mg, 1.08 mmol, 150 μL, 3.00 eq). Then the mixture was stirred at 25° C. for 2 h. The pH of the reaction mixture was adjusted to 5-6 by formic acid (0.2 mL) and filtered. The filtrate was purified by reversed-phase HPLC (column: Phenomenex Synergi C18 150*25 mm*10 um; mobile phase: [water (0.225% formic acid)-acetonitrile]; B %: 35%-68%, 11 min) and lyophilized to afford N-(2-(2,6-dioxopiperidin-3-yl)-1-oxoisoindolin-5-yl)-3-isopropylindoline-1-carboxamide (31.46 mg, 69.8 umol, 19% yield, 99% purity) as a white solid.

¹H NMR (400 MHz, DMSO-d₆) δ=10.98 (s, 1H), 8.86 (s, 1H), 7.96-7.88 (m, 2H), 7.70-7.64 (m, 2H), 7.23 (br d, J=7.4 Hz, 1H), 7.17 (t, J=7.8 Hz, 1H), 6.99-6.93 (m, 1H), 5.10 (dd, J=5.1, 13.1 Hz, 1H), 4.48-4.40 (m, 1H), 4.35-4.27 (m, 1H), 4.15-4.08 (m, 1H), 4.06-4.00 (m, 1H), 3.44-3.41 (m, 1H), 2.98-2.87 (m, 1H), 2.58 (br s, 1H), 2.47-2.37 (m, 1H), 2.11-1.99 (m, 2H), 0.99 (d, J=6.8 Hz, 3H), 0.76 (d, J=6.8 Hz, 3H).

### Example 42. Synthesis of Compound 210

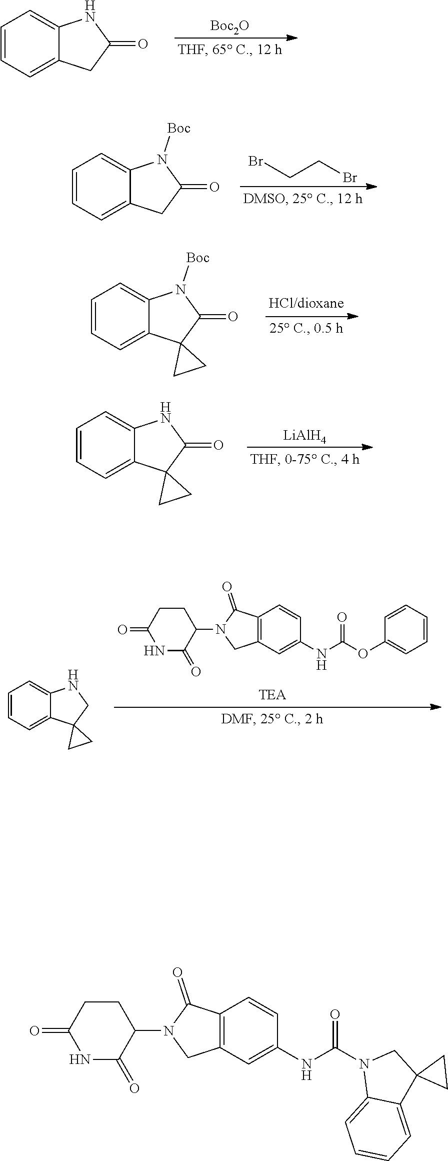

Step 1. To a solution of indolin-2-one (2.00 g, 15.0 mmol, 1.00 eq) in tetrahydrofuran (60.0 mL) was added sodium carbonate (12.7 g, 120 mmol, 8.00 eq) and di-tert-butyl dicarbonate (4.92 g, 22.5 mmol, 5.18 mL, 1.50 eq). Then the mixture was stirred at 65° C. for 12 h. The mixture was concentrated in vacuum. The residue was diluted with water (50 mL) and extracted with ethyl acetate (3×20 mL). The combined organic layers were washed with brine (3×10 mL), dried over anhydrous sodium sulfate, filtered and concentrated under reduced pressure to give a residue. The residue was purified by column chromatography (SiO₂, petroleum ether/ethyl acetate=50/1 to 10/1) to afford the tert-butyl 2-oxoindoline-1-carboxylate (2.40 g, 10.1 mmol, 67% yield, 99% purity) as a white solid.

¹H NMR (400 MHz, DMSO-d₆) δ=7.70 (d, J=8.3 Hz, 1H), 7.36-7.26 (m, 2H), 7.20-7.12 (m, 1H), 3.75 (s, 2H), 1.57 (s, 9H).

Step 2. To a solution of tert-butyl 2-oxoindoline-1-carboxylate (2.00 g, 8.57 mmol, 1.00 eq) in dimethylsulfoxide (20.0 mL) was added potassium carbonate (4.74 g, 34.3 mmol, 4.00 eq), 1,2-dibromoethane (2.42 g, 12.8 mmol, 970 μL, 1.50 eq). The resulting mixture was stirred at 25° C. for 12 h, and then the reaction mixture was filtered. The filtrated was purified by reversed phase column chromatography (C18, 40 g; condition: water/acetonitrile=1/0 to 0/1, 0.1% formic acid) and lyophilized to afford tert-butyl 2′-oxospiro[cyclopropane-1,3′-indoline]-1′-carboxylate (280 mg, 1.03 mmol, 11% yield, 95% purity) as a yellow solid.

¹H NMR (400 MHz, DMSO-d₆) δ=7.78 (d, J=8.1 Hz, 1H), 7.30 (dt, J=1.4, 7.8 Hz, 1H), 7.16 (dt, J=0.9, 7.5 Hz, 1H), 7.07 (dd, J=0.9, 7.4 Hz, 1H), 1.71-1.67 (m, 2H), 1.64 (t, J=2.9 Hz, 2H), 1.58 (s, 9H).

Step 3. To a solution of 2′-oxospiro[cyclopropane-1,3′-indoline]-1′-carboxylate (280 mg, 1.08 mmol, 1.00 eq) in dioxane (1.00 mL) was added hydrochloric acid/dioxane (4 M, 25.4 mL, 94.2 eq). Then the mixture was stirred at 25° C. for 0.5 h. The reaction mixture was concentrated in vacuum to afford spiro[cyclopropane-1,3′-indolin]-2′-one (250 mg, crude) as red oil. MS (ESI) m/z 160.1 [M+H]⁺

Step 4. To a solution of spiro[cyclopropane-1,3′-indoline]-2′-one (250 mg, 1.57 mmol, 1.00 eq) in tetrahydrofuran (30.0 mL) was added lithium aluminum hydride (89.4 mg, 2.36 mmol, 1.50 eq) at 0° C. under nitrogen atmosphere. Then the mixture was stirred at 75° C. for 4 h. The reaction mixture was quenched by sodium hydroxide (6M, 20 mL) and extracted with ethyl acetate (3×30 mL). The combined organic layers were washed with brine (3×10 mL), dried over anhydrous sodium sulfate, filtered and concentrated under reduced pressure to give spiro[cyclopropane-1,3′-indoline](200 mg, crude) as red oil. MS (ESI) m/z 146.1 [M+H]⁺

Step 5. To a solution of spiro[cyclopropane-1,3′-indoline](76.5 mg, 527 umol, 2.00 eq) and phenyl (2-(2,6-dioxopiperidin-3-yl)-1-oxoisoindolin-5-yl)carbamate (100 mg, 263 umol, 1.00 eq) in N,N-dimethyl formamide (2.00 mL) was added triethylamine (80.0 mg, 790 umol, 110 μL, 3.00 eq). Then the mixture was stirred at 25° C. for 2 h. The reaction mixture was adjust pH to 5-6 by formic acid (0.200 mL) and filtered. The filtrate was purified by reversed phase column chromatography (C18, 40 g; condition: water/acetonitrile=1/0 to 0/1, 0.1% formic acid) and lyophilized to afford N-(2-(2,6-dioxopiperidin-3-yl)-1-oxoisoindolin-5-yl)spiro[cyclopropane-1,3′-indoline]-1′-carboxamide (26.8 mg, 61.6 umol, 23% yield, 99% purity) as a white solid.

¹H NMR (400 MHz, DMSO-d₆) δ=11.06-10.93 (m, 1H), 8.88-8.74 (m, 1H), 8.48 (s, 0.2H), 7.92 (s, 1H), 7.87 (d, J=7.9 Hz, 1H), 7.64 (s, 2H), 7.16-7.07 (m, 1H), 6.96-6.88 (m, 1H), 6.79 (d, J=6.8 Hz, 1H), 5.14-5.05 (m, 1H), 4.48-4.40 (m, 1H), 4.34-4.26 (m, 1H), 4.20 (s, 2H), 2.98-2.85 (m, 1H), 2.64-2.57 (m, 1H), 2.41-2.37 (m, 1H), 2.06-1.96 (m, 1H), 1.15-1.05 (m, 4H).

### Example 42. Synthesis of Compound 192

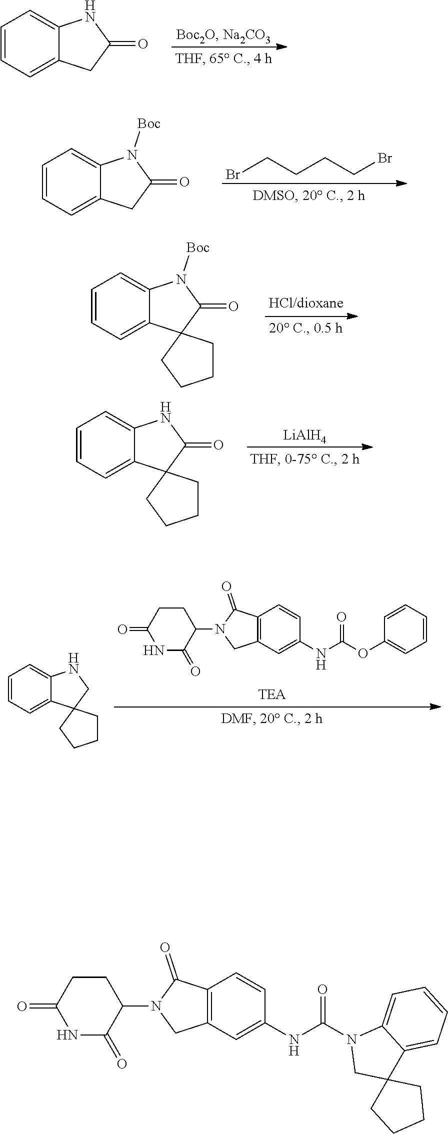

Step 1. To a solution of indolin-2-one (3.00 g, 22.5 mmol, 1.00 eq) in tetrahydrofuran (70.0 mL) was added sodium carbonate (19.1 g, 180 mmol, 8.00 eq) and di-tert-butyl dicarbonate (7.38 g, 33.8 mmol, 7.76 mL, 1.50 eq). The mixture was stirred at 65° C. for 4 h. The reaction mixture was concentrated under reduced pressure to give a residue. The residue was purified by column chromatography (SiO₂, petroleum ether/ethyl acetate=8/1 to 0/1) to give tert-butyl 2-oxoindoline-1-carboxylate (6.00 g, crude) as a white solid.

¹H NMR (400 MHz, DMSO-d₆) δ=7.69 (d, J=8.4 Hz, 1H), 7.33-7.26 (m, 2H), 7.17-7.11 (m, 1H), 3.74 (s, 2H), 1.56 (s, 9H).

Step 2. To a solution of tert-butyl 2-oxoindoline-1-carboxylate (1.50 g, 6.43 mmol, 1.00 eq) in dimethylsulfoxide (10.0 mL) was added 1,4-dibromobutane (1.53 g, 7.07 mmol, 853 uL, 1.10 eq) and potassium carbonate (2.67 g, 19.2 mmol, 3.00 eq). The mixture was stirred at 20° C. for 2 h. The mixture was filtered. The filtrate was purified by reversed-phase HPLC (column: spherical C18, 20-45 um, 100 Å, SW 330, mobile phase: [water (0.1% Formic Acid)-ACN]) to give tert-butyl 2′-oxospiro[cyclopentane-1,3′-indoline]-1′-carboxylate (1.40 g, 4.87 mmol, 75% yield) as yellow oil.

¹H NMR (400 MHz, DMSO-d₆) δ=7.71 (d, J=8.1 Hz, 1H), 7.36 (dd, J=0.8, 7.4 Hz, 1H), 7.30 (dt, J=1.4, 7.9 Hz, 1H), 7.22-7.15 (m, 1H), 2.09-2.05 (m, 2H), 1.99-1.89 (m, 4H), 1.87-1.78 (m, 2H), 1.57 (s, 9H).

Step 3. A mixture of tert-butyl 2′-oxospiro[cyclopentane-1,3′-indoline]-1′-carboxylate (1.40 g, 4.87 mmol, 1.00 eq) in hydrochloric acid/dioxane (4 M, 20.0 mL, 16.4 eq) was stirred at 20° C. for 0.5 h. The reaction mixture was concentrated under reduced pressure to give spiro[cyclopentane-1,3′-indolin]-2′-one (0.900 g, 4.81 mmol, 98% yield) as a purple solid.

¹H NMR (400 MHz, DMSO-d₆) δ=10.25 (br s, 1H), 7.22 (d, J=7.4 Hz, 1H), 7.17-7.11 (m, 1H), 6.96-6.91 (m, 1H), 6.81 (d, J=7.6 Hz, 1H), 1.98-1.90 (m, 6H), 1.77-1.70 (m, 2H).

Step 4. To a solution of spiro[cyclopentane-1,3′-indolin]-2′-one (0.200 g, 1.07 mmol, 1.00 eq) in tetrahydrofuran (30.0 mL) was added lithium aluminum hydride (60.8 mg, 1.60 mmol, 1.50 eq) at 0° C. under nitrogen. The mixture was stirred at 75° C. for 2 h. 620 mg sodium sulfate decahydrate was added to the reaction mixture and stirred at 20° C. for 10 min and filtered. The filtrate was concentrated under reduced pressure to give spiro[cyclopentane-1,3′-indoline](170 mg, 981 umol, 91% yield) as pink oil.

¹H NMR (400 MHz, DMSO-d₆) δ=6.97 (d, J=7.3 Hz, 1H), 6.89 (dt, J=1.3, 7.5 Hz, 1H), 6.53 (dt, J=0.9, 7.3 Hz, 1H), 6.47 (d, J=7.8 Hz, 1H), 5.40 (br s, 1H), 3.20 (d, J=2.1 Hz, 2H), 1.78-1.64 (m, 8H).

Step 5. To a solution of phenyl (2-(2,6-dioxopiperidin-3-yl)-1-oxoisoindolin-5-yl)carbamate (140 mg, 369 umol, 1.00 eq) in dimethyl formamide (1.00 mL) was added triethylamine (112 mg, 1.11 mmol, 154 μL, 3.00 eq) and spiro[cyclopentane-1,3′-indoline](76.7 mg, 442 umol, 1.20 eq). The mixture was stirred at 20° C. for 2 h. The mixture was filtered to give a filter liquor. The filter liquor was purified by reversed-phase HPLC (column: spherical C18, 20-45 um, 100 Å, SW 40, mobile phase: [water (0.1% Formic Acid)-ACN]) to give N-(2-(2,6-dioxopiperidin-3-yl)-1-oxoisoindolin-5-yl)spiro[cyclopentane-1,3′-indoline]-1′-carboxamide (102.84 mg, 224 umol, 61% yield) as a white solid.

¹H NMR (400 MHz, DMSO-d₆) δ=10.98 (s, 1H), 8.83 (s, 1H), 7.92 (s, 1H), 7.88 (d, J=8.0 Hz, 1H), 7.69-7.62 (m, 2H), 7.24 (d, J=7.3 Hz, 1H), 7.19-7.12 (m, 1H), 7.00-6.94 (m, 1H), 5.09 (dd, J=5.2, 13.3 Hz, 1H), 4.47-4.39 (m, 1H), 4.33-4.25 (m, 1H), 3.98 (s, 2H), 2.97-2.87 (m, 1H), 2.60 (br dd, J=1.8, 15.7 Hz, 1H), 2.45-2.35 (m, 1H), 2.03-1.97 (m, 1H), 1.85 (br d, J=6.3 Hz, 6H), 1.80-1.73 (m, 2H).

### Example 43. Synthesis of Compound 117

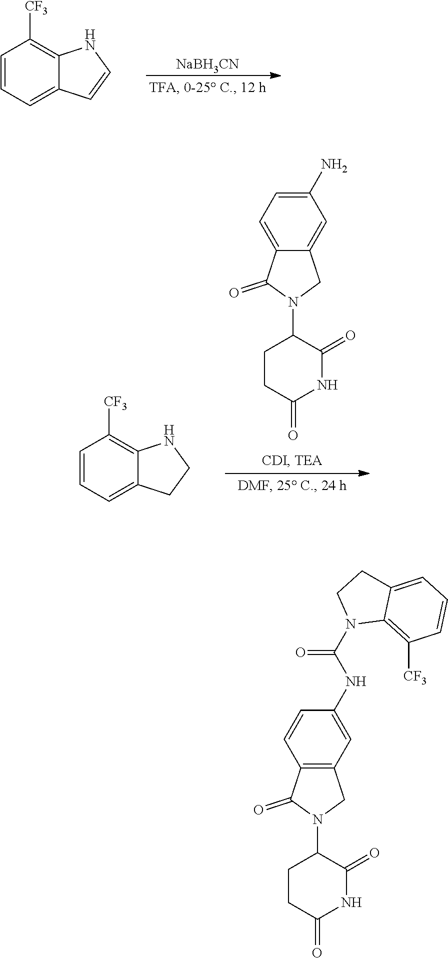 [SMILES: FC(F)(F)c1cccc2cc[nH]c12 ; "NaBH3CN" ; FC(F)(F)c1cccc2c1NCC2 ; Nc1ccc2c(c1)CN(C1CCC(=O)NC1=O)C2=O ; O=C1CCC(N2Cc3cc(NC(=O)N4CCc5cccc(C(F)(F)F)c54)ccc3C2=O)C(=O)N1]

Step 1. To a solution of 7-(trifluoromethyl)-1H-indole (500 mg, 2.70 mmol, 1.00 eq) in trifluoroacetic acid (5.00 mL) was added sodium cyanoborohydride (254 mg, 4.05 mmol, 1.50 eq) at 0° C. Then the mixture was stirred at 25° C. for 12 h. The reaction mixture was diluted with water (10.0 mL) and exacted with ethyl acetate (30.0 mL). The organic phase was separated, washed with brine (10.0 mL), dried over anhydrous sodium sulfate, filtered and concentrated under reduced pressure to afford 7-(trifluoromethyl)indoline (500 mg, crude) as yellow oil.

Step 2. To a solution of 3-(5-amino-1-oxoisoindolin-2-yl)piperidine-2,6-dione (124 mg, 480 umol, 1.80 eq) and 7-(trifluoromethyl)indoline (50.0 mg, 267 umol, 1.00 eq) in N,N-dimethyl formamide (4.00 mL) was added triethylamine (81.1 mg, 801 umol, 111 μL, 3.00 eq) and 1,1′-carbonyldiimidazole (649 mg, 4.01 mmol, 15.0 eq). The mixture was stirred at 25° C. for 24 h, and then the mixture was filtered. The filtrate purified by Prep-HPLC (column: Phenomenex luna C18 150*25 mm*10 um; mobile phase: [water (FA)-ACN]; B %: 24%-54%, 8 min). The desired fraction was collected and lyophilized to afford N-(2-(2,6-dioxopiperidin-3-yl)-1-oxoisoindolin-5-yl)-7-(trifluoromethyl)indoline-1-carboxamide (66.3 mg, 137 umol, 51% yield, 98% purity) as a white solid.

¹H NMR (400 MHz, DMSO-d₆) δ=10.98 (s, 1H), 9.80 (s, 1H), 7.82 (s, 1H), 7.70-7.62 (m, 1H), 7.60-7.50 (m, 2H), 7.45 (d, J=7.9 Hz, 1H), 7.25-7.18 (m, 1H), 5.08 (dd, J=5.1, 13.3 Hz, 1H), 4.49-4.37 (m, 1H), 4.31-4.25 (m, 1H), 4.16 (br t, J=7.9 Hz, 2H), 3.13 (br t, J=7.9 Hz, 2H), 2.99-2.87 (m, 1H), 2.61 (br d, J=2.4 Hz, 1H), 2.40-2.31 (m, 1H), 2.05-1.93 (m, 1H).

### Example 44. Synthesis of Compound 147

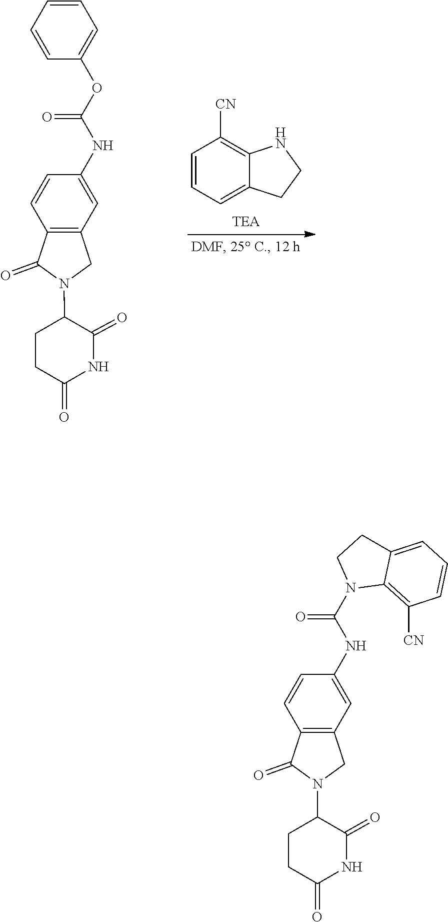 [SMILES: O=C1CCC(N2Cc3cc(NC(=O)Oc4ccccc4)ccc3C2=O)C(=O)N1 ; N#Cc1cccc2c1NCC2 ; N#Cc1cccc2c1N(C(=O)Nc1ccc3c(c1)CN(C1CCC(=O)NC1=O)C3=O)CC2]

To a solution of phenyl (2-(2,6-dioxopiperidin-3-yl)-1-oxoisoindolin-5-yl)carbamate (300 mg, 790 umol, 1.00 eq) and indoline-7-carbonitrile (114 mg, 790 umol, 1.00 eq) in N,N-dimethyl formamide (1.00 mL) was added triethylamine (240 mg, 2.37 mmol, 330 μL, 3.00 eq). Then the mixture was stirred at 25° C. for 12 h. The reaction mixture was diluted with water (30 mL) and extracted with ethyl acetate (2×30 mL). The organic phase was separated, washed with brine (10 mL), dried over sodium sulfate, filtered and lyophilized to give a yellow solid. The yellow solid was dissolved in N,N-dimethyl formamide (5.00 mL) and purified by Prep-HPLC (column: Phenomenex luna C18 150*25 mm*10 um; mobile phase: [water (FA)-ACN]; B %: 0%-20%, 10 min), Prep-NPLC (column: Welch Ultimate XB—CN 250*25*10 um; mobile phase: [Heptane-EtOH]; B %: 15%-100%, 0 min) and Prep-HPLC (column: Phenomenex luna C18 150*25 mm*10 um; mobile phase: [water (FA)-ACN]; B %: 0%-23%, 10 min). The desired fraction was collected and lyophilized to afford 7-cyano-N-(2-(2,6-dioxopiperidin-3-yl)-1-oxoisoindolin-5-yl)indoline-1-carboxamide (12.3 mg, 28.4 umol, 3% yield, 99% purity) as a white solid.

¹H NMR (400 MHz, DMSO-d₆) δ=11.02 (s, 1H), 8.19 (s, 0.7H), 7.83 (dd, J=3.0, 7.6 Hz, 2H), 7.53 (s, 1H), 7.48 (d, J=7.1 Hz, 1H), 7.40 (d, J=8.1 Hz, 1H), 7.13 (t, J=7.6 Hz, 1H), 5.15 (dd, J=5.1, 13.3 Hz, 1H), 4.55-4.48 (m, 1H), 4.42-4.35 (m, 1H), 4.13 (br t, J=8.3 Hz, 2H), 3.31-3.31 (m, 2H), 2.96-2.90 (m, 1H), 2.63-2.59 (m, 1H), 2.42 (br dd, J=4.1, 13.3 Hz, 1H), 2.04 (br dd, J=5.1, 10.9 Hz, 1H).

### Example 45. Synthesis of Compound 148

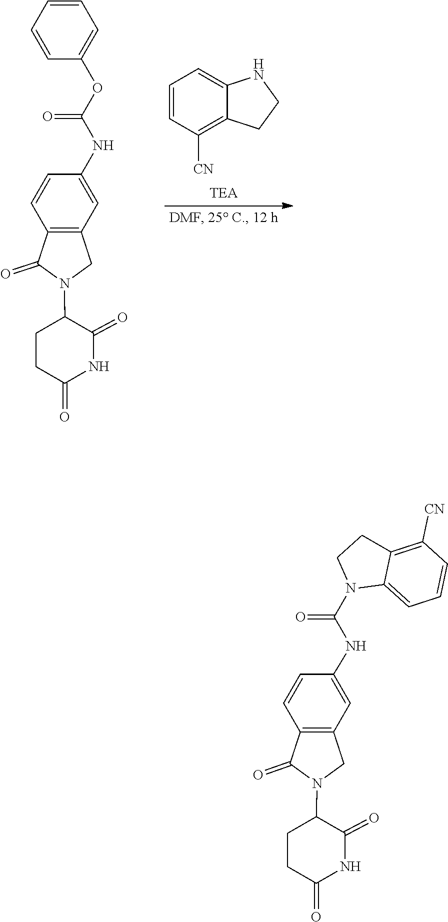 [SMILES: O=C1CCC(N2Cc3cc(NC(=O)Oc4ccccc4)ccc3C2=O)C(=O)N1 ; N#Cc1cccc2c1CCN2 ; N#Cc1cccc2c1CCN2C(=O)Nc1ccc2c(c1)CN(C1CCC(=O)NC1=O)C2=O]

Step 1. To a solution of phenyl (2-(2,6-dioxopiperidin-3-yl)-1-oxoisoindolin-5-yl)carbamate (150 mg, 395 umol, 1.00 eq) and triethylamine (120 mg, 1.19 mmol, 165 μL, 3.00 eq) in dimethyl formamide (3.00 mL) was added indoline-4-carbonitrile (57.0 mg, 395 umol, 1.00 eq). The mixture was stirred at 25° C. for 12 h. The mixture was filtered. The filter liquor was purified by Prep-HPLC (column: Phenomenex luna C18 150*25 mm*10 um; mobile phase: [water (formic acid)-acetonitrile]; B %: 32%-62%, 10 min) to give 4-cyano-N-(2-(2,6-dioxopiperidin-3-yl)-1-oxoisoindolin-5-yl)indoline-1-carboxamide (25.1 mg, 58.4 umol, 14% yield) as a yellow solid.

¹H NMR (400 MHz, DMSO-d₆) δ=10.98 (s, 1H), 8.99 (s, 1H), 8.18-8.10 (m, 1H), 7.91 (s, 1H), 7.66 (d, J=0.9 Hz, 2H), 7.37-7.32 (m, 2H), 5.09 (dd, J=5.1, 13.4 Hz, 1H), 4.48-4.40 (m, 1H), 4.31 (d, J=9.9 Hz, 1H), 4.29-4.24 (m, 2H), 3.38 (br t, J=8.6 Hz, 2H), 2.97-2.86 (m, 1H), 2.62 (br s, 1H), 2.39 (br dd, J=4.2, 12.8 Hz, 1H), 2.05-1.96 (m, 1H).

### Example 46. Synthesis of Compound 152

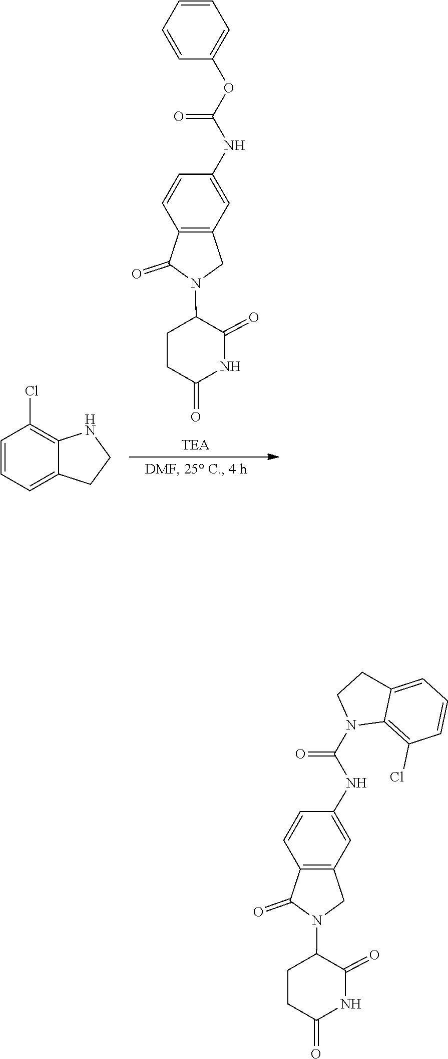 [SMILES: Clc1cccc2c1NCC2 ; O=C1CCC(N2Cc3cc(NC(=O)Oc4ccccc4)ccc3C2=O)C(=O)N1 ; O=C1CCC(N2Cc3cc(NC(=O)N4CCc5cccc(Cl)c54)ccc3C2=O)C(=O)N1]

To a solution of phenyl (2-(2,6-dioxopiperidin-3-yl)-1-oxoisoindolin-5-yl)carbamate (246 mg, 651 umol, 1.00 eq) and triethylamine (197 mg, 1.95 mmol, 271 μL, 3.00 eq) in dimethyl formamide (2.00 mL) was added 7-chloroindoline (100 mg, 651 umol, 1.00 eq). The mixture was stirred at 25° C. for 4 h. The mixture was filtered. The filtrate was purified by reversed-phase HPLC (column: spherical C18, 20-45 um, 100 Å, SW 80, mobile phase: [water-acetonitrile) to give 7-chloro-N-(2-(2,6-dioxopiperidin-3-yl)-1-oxoisoindolin-5-yl) indoline-1-carboxamide (23.4 mg, 53.3 umol, 8% yield) as an off-white solid.

¹H NMR (400 MHz, DMSO-d₆) δ=10.96 (s, 1H), 9.72 (s, 1H), 7.84 (s, 1H), 7.66-7.62 (m, 1H), 7.61-7.56 (m, 1H), 7.24 (d, J=7.3 Hz, 1H), 7.22 (d, J=8.1 Hz, 1H), 7.07-7.02 (m, 1H), 5.08 (dd, J=5.1, 13.3 Hz, 1H), 4.45-4.39 (m, 1H), 4.32-4.24 (m, 1H), 4.13 (t, J=7.9 Hz, 2H), 3.14 (br t, J=7.8 Hz, 2H), 2.96-2.86 (m, 1H), 2.61 (br d, J=2.3 Hz, 1H), 2.44-2.35 (m, 1H), 2.04-1.94 (m, 1H).

### Example 47. Synthesis of Compound 162

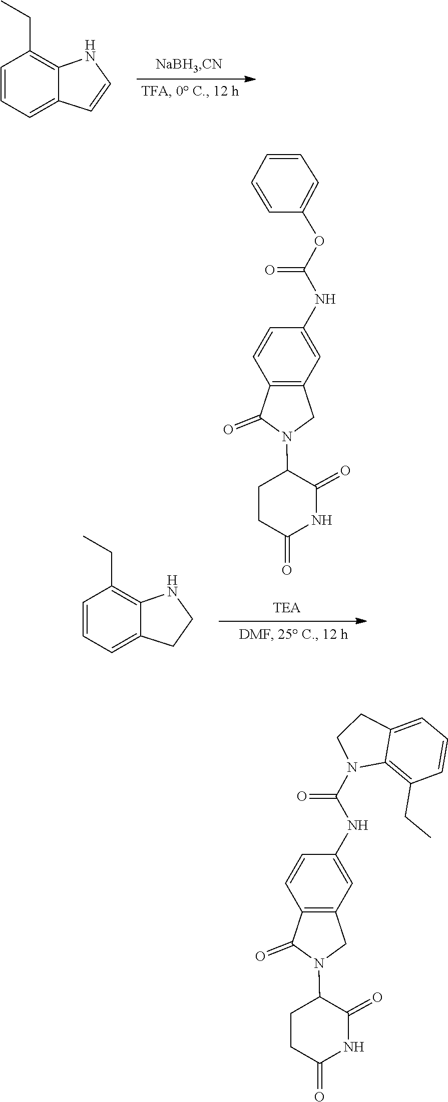 [SMILES: CCc1cccc2cc[nH]c12 ; CCc1cccc2c1NCC2 ; O=C1CCC(N2Cc3cc(NC(=O)Oc4ccccc4)ccc3C2=O)C(=O)N1 ; CCc1cccc2c1N(C(=O)Nc1ccc3c(c1)CN(C1CCC(=O)NC1=O)C3=O)CC2]

Step 1. To a solution of 7-ethyl-1H-indole (500 mg, 3.44 mmol, 472 μL, 1.00 eq) in trifluoroacetic acid (5.00 mL) was added sodium cyanoborohydride (325 mg, 5.17 mmol, 1.50 eq) at 0° C. The mixture was stirred at 25° C. for 0.5 h. The mixture was diluted with water (200 mL) and extracted with dichloromethane (3×50.0 mL). The organic layers were collected and was washed with brine (50.0 mL), dried over anhydrous sodium sulfate and evaporated to afford 7-ethylindoline (400 mg, crude) as white oil.

Step 2. To a solution of 7-ethylindoline (38.8 mg, 264 umol, 1.00 eq) and phenyl (2-(2,6-dioxopiperidin-3-yl)-1-oxoisoindolin-5-yl)carbamate (100 mg, 264 umol, 1 eq) in N,N-dimethyl formamide (2.00 mL) was added triethylamine (80.0 mg, 791 umol, 110 μL, 3.00 eq). The mixture was stirred at 25° C. for 12 h. The mixture was filtered and collected the filtrate, which was purified by reversed-phase HPLC (column: spherical C18, 20-45 um, 100 Å, SW 40, mobile phase: [water (0.1% Formic Acid)-ACN]; B %: 15%-60%, 30 min) and Prep-HPLC (column: Phenomenex Luna C18 200*40 mm*10 um; mobile phase: [water (FA)-ACN]; B %: 25%-55%, 10 min). The desired fraction was collected and lyophilized to afford N-(2-(2,6-dioxopiperidin-3-yl)-1-oxoisoindolin-5-yl)-7-ethylindoline-1-carboxamide (23.13 mg, 53.5 umol, 20% yield) as an off-white solid. ¹H NMR (400 MHz, DMSO-d₆) δ=10.97 (s, 1H), 9.56 (s, 1H), 7.82 (s, 1H), 7.65-7.61 (m, 1H), 7.59-7.56 (m, 1H), 7.10 (d, J=6.5 Hz, 1H), 7.06-7.03 (m, 1H), 7.01 (d, J=7.1 Hz, 1H), 5.08 (dd, J=5.0, 13.3 Hz, 1H), 4.45-4.39 (m, 1H), 4.30-4.24 (m, 1H), 4.10 (t, J=7.8 Hz, 2H), 3.04 (br t, J=7.5 Hz, 2H), 2.92-2.86 (m, 1H), 2.61 (br d, J=2.1 Hz, 1H), 2.56 (br s, 2H), 2.37 (br dd, J=4.7, 13.3 Hz, 1H), 2.02-1.96 (m, 1H), 1.10 (t, J=7.6 Hz, 3H)

### Example 48. Synthesis of Compound 164

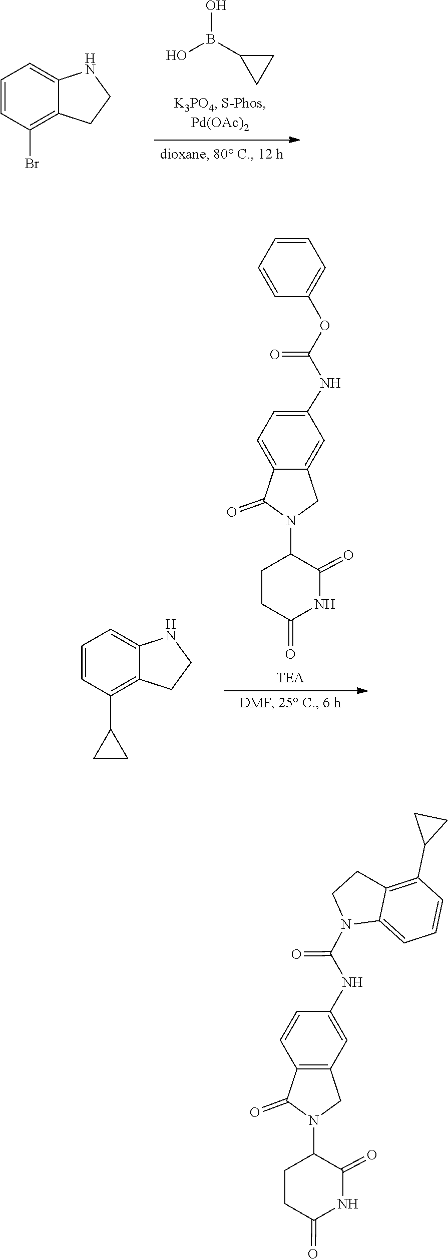 [SMILES: Brc1cccc2c1CCN2 ; OB(O)C1CC1 ; c1cc2c(c(C3CC3)c1)CCN2 ; O=C1CCC(N2Cc3cc(NC(=O)Oc4ccccc4)ccc3C2=O)C(=O)N1 ; O=C1CCC(N2Cc3cc(NC(=O)N4CCc5c(C6CC6)cccc54)ccc3C2=O)C(=O)N1]

Step 1. To a solution of 4-bromoindoline (500 mg, 2.52 mmol, 1.00 eq), cyclopropylboronic acid (260 mg, 3.03 mmol, 1.20 eq) and potassium phosphate (1.61 g, 7.57 mmol, 3.00 eq) in 1,4-dioxane (5.00 mL) was added dicyclohexyl(2′,6′-dimethoxy-[1,1′-biphenyl]-2-yl)phosphine (207 mg, 504 umol, 0.200 eq) and diacetoxypalladium (113 mg, 504 umol, 0.200 eq). The mixture was stirred at 80° C. for 12 h under nitrogen. The mixture was filtered. The filtrate was concentrated under reduced pressure to give a residue, which was purified by reversed-phase HPLC (column: spherical C18, 20-45 um, 100 Å, SW 40, mobile phase: [water (0.1% Formic Acid)-acetonitrile) to give 4-cyclopropylindoline (36.0 mg, 226 umol, 8% yield) as a yellow solid.

¹H NMR (400 MHz, DMSO-d₆) δ=6.78 (t, J=7.7 Hz, 1H), 6.27 (d, J=7.6 Hz, 1H), 6.03 (d, J=7.8 Hz, 1H), 5.38 (br s, 1H), 3.43-3.39 (m, 2H), 2.94 (t, J=8.5 Hz, 2H), 1.78-1.70 (m, 1H), 0.89-0.82 (m, 2H), 0.62-0.54 (m, 2H).

Step 2. To a solution of phenyl (2-(2,6-dioxopiperidin-3-yl)-1-oxoisoindolin-5-yl)carbamate (94.3 mg, 248 umol, 1.10 eq) and triethylamine (68.6 mg, 678 umol, 94.4 uL, 3.00 eq) in dimethyl formamide (1.00 mL) was added 4-cyclopropylindoline (36.0 mg, 226 umol, 1.00 eq) at 25° C. The mixture was stirred at 25° C. for 6 h. The mixture was filtered to give a filter liquor, which was purified by prep-HPLC (column: Phenomenex luna C18 150*25 mm*10 um; mobile phase: [water (Formic Acid)-acetonitrile]; B %: 22%-52%, 10 min) to give 4-cyclopropyl-N-(2-(2,6-dioxopiperidin-3-yl)-1-oxoisoindolin-5-yl)indoline-1-carboxamide (13.0 mg, 29.2 umol, 12% yield) as a white solid.

¹H NMR (400 MHz, DMSO-d₆) δ=10.98 (s, 1H), 8.82 (s, 1H), 7.92 (s, 1H), 7.69 (d, J=8.0 Hz, 1H), 7.65 (s, 2H), 7.05 (t, J=7.9 Hz, 1H), 6.47 (d, J=7.8 Hz, 1H), 5.09 (dd, J=4.8, 12.9 Hz, 1H), 4.47-4.39 (m, 1H), 4.32-4.26 (m, 1H), 4.20 (br t, J=8.5 Hz, 2H), 3.25 (br t, J=8.4 Hz, 2H), 2.97-2.87 (m, 1H), 2.63-2.57 (m, 1H), 2.39 (br dd, J=4.4, 12.5 Hz, 1H), 2.04-1.97 (m, 1H), 1.88-1.79 (m, 1H), 0.96-0.90 (m, 2H), 0.69-0.63 (m, 2H).

### Example 49. Synthesis of Compound 171

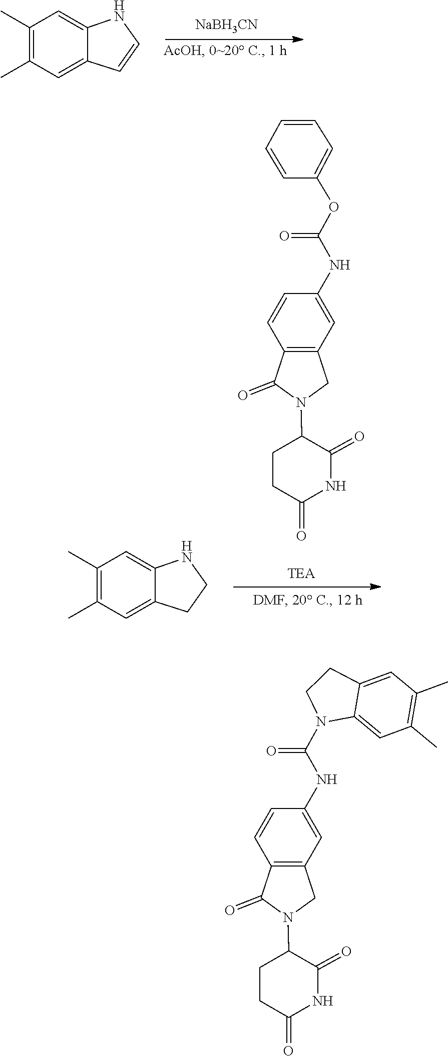 [SMILES: Cc1cc2cc[nH]c2cc1C ; "NaBH3CN" ; O=C1CCC(N2Cc3cc(NC(=O)Oc4ccccc4)ccc3C2=O)C(=O)N1 ; Cc1cc2c(cc1C)NCC2 ; Cc1cc2c(cc1C)N(C(=O)Nc1ccc3c(c1)CN(C1CCC(=O)NC1=O)C3=O)CC2]

Step 1. To a solution of 5,6-dimethyl-1H-indole (500 mg, 3.44 mmol, 1.00 eq) in acetic acid (2.00 mL) was added sodium cyanoborohydride (324 mg, 5.17 mmol, 1.50 eq) at 0° C. The mixture was stirred at 20° C. for 1 h. The reaction mixture was concentrated under reduced pressure to give a residue. The crude product was purified by reversed-phase HPLC (column: spherical C18, 20-45 um, 100 Å, SW 80, mobile phase: [water (0.1% Formic Acid)-ACN]) to give 5,6-dimethylindoline (0.400 g, 2.72 mmol, 78% yield) as a yellow solid.

¹H NMR (400 MHz, DMSO-d₆) δ=6.92 (s, 1H), 6.58 (s, 1H), 3.46-3.43 (m, 2H), 2.89 (t, J=8.2 Hz, 2H), 2.12 (s, 3H), 2.11 (s, 3H).

Step 2. To a solution of phenyl (2-(2,6-dioxopiperidin-3-yl)-1-oxoisoindolin-5-yl)carbamate (80.0 mg, 210 umol, 1.00 eq) in dimethyl formamide (1.00 mL) was added triethylamine (64.0 mg, 632 umol, 88.0 uL, 3.00 eq) and 5,6-dimethylindoline (34.1 mg, 231 umol, 1.10 eq). The mixture was stirred at 20° C. for 12 h. The mixture was filtered to give a filter cake, then concentrated under reduced pressure to give a residue, which was triturated with dimethylsulfoxide (2.00 mL) and filtered. The filter cake was washed with water (2.00 mL) and dried to give N-(2-(2,6-dioxopiperidin-3-yl)-1-oxoisoindolin-5-yl)-5,6-dimethylindoline-1-carboxamide (17.86 mg, 41.3 umol, 20% yield) as an off-white solid. ¹H NMR (400 MHz, DMSO-d₆) δ=10.98 (s, 1H), 8.78 (s, 1H), 7.94 (s, 1H), 7.72 (s, 1H), 7.64 (s, 2H), 6.98 (s, 1H), 5.10 (dd, J=5.0, 13.3 Hz, 1H), 4.47-4.39 (m, 1H), 4.34-4.25 (m, 1H), 4.14 (br t, J=8.5 Hz, 2H), 3.11 (br t, J=8.4 Hz, 2H), 2.97-2.87 (m, 1H), 2.61 (br d, J=16.9 Hz, 1H), 2.40 (dq, J=4.3, 13.2 Hz, 1H), 2.19 (s, 3H), 2.16 (s, 3H), 2.05-1.96 (m, 1H).

### Example 50. Synthesis of Compound 325

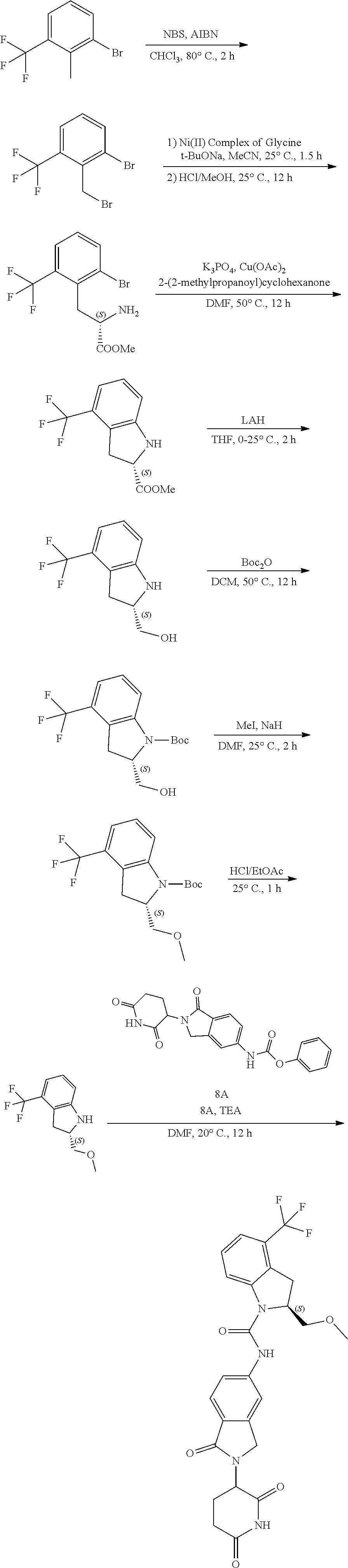

Step 1. To a solution of 1-bromo-2-methyl-3-(trifluoromethyl)benzene (5.00 g, 20.9 mmol, 1.00 eq) in trichloromethane (50.0 mL) was added N-Bromosuccinimide (4.10 g, 23.0 mmol, 1.10 eq) and (E)-2,2′-(diazene-1,2-diyl)bis(2-methylpropanenitrile) (3.43 g, 20.9 mmol, 1.00 eq), the mixture was stirred at 80° C. for 12 h. The reaction mixture was concentrated under reduced pressure to give a residue. The residue was purified by column chromatography (SiO2, Petroleum ether/Ethyl acetate=1/0 to 10/1) to afford 1-bromo-2-(bromomethyl)-3-(trifluoromethyl)benzene (2.50 g, 7.86 mmol, 38% yield) as yellow oil. ¹H NMR (400 MHz, DMSO-d₆) δ=8.05 (br d, J=8.1 Hz, 1H), 7.83 (br d, J=7.8 Hz, 1H), 7.52 (t, J=8.0 Hz, 1H), 4.76 (s, 2H).

Step 2. To a solution of 1-bromo-2-(bromomethyl)-3-(trifluoromethyl)benzene (2.30 g, 7.23 mmol, 1.00 eq) in acetonitrile (30.0 mL) was added sodium tert-butoxide (834 mg, 8.68 mmol, 1.20 eq) and (S)-(o-(Nbenzylprolyl)amino)(phenyl)methyleneiminoacetate (3.60 g, 7.23 mmol, 1.00 eq), the mixture was stirred at 25° C. for 1.5 h. The reaction mixture was diluted with water (100 mL) and exacted with ethyl acetate (3×100 mL). The organic phase was separated, washed with brine (2×100 mL), dried over sodium sulfate, filtered and concentrated under reduced pressure to give a residue. Then hydrochloric acid (4 M in methanol) (50.0 mL) was added, the mixture was stirred at 70° C. for 12 h. The mixture was filtered to give filtrate, then was concentrated under reduced pressure to give a residue. The residue was purified by reversed phase-HPLC (FA condition) to give methyl (S)-2-amino-3-(2-bromo-6-(trifluoromethyl)phenyl)propanoate (3.00 g, crude) as a white solid. ¹H NMR (400 MHz, DMSO-d₆) δ=9.02 (br d, J=3.0 Hz, 2H), 8.00 (br s, 1H), 7.78 (br s, 1H), 7.50-7.42 (m, 1H), 4.23 (br d, J=4.9 Hz, 1H), 3.58-3.44 (m, 2H), 3.43 (br d, J=3.6 Hz, 3H).

Step 3. To a solution of methyl (S)-2-amino-3-(2-bromo-6-(trifluoromethyl)phenyl) propanoate (300 mg, 920 mol, 1.00 eq) in dimethylformamide (5.00 mL) was added 2-(2-methylpropanoyl)cyclohexan-1-one (31.0 mg, 184 mol, 0.200 eq), potassium phosphate (391 mg, 1.84 mmol, 2.00 eq) and copper(II)acetate (8.35 mg, 46.0 mol, 0.0500 eq), the mixture was stirred at 50° C. for 12 h under nitrogen atmosphere. The reaction mixture was diluted with water (50.0 mL) and exacted with ethyl acetate (3×50.0 mL). The organic phase was separated, washed with brine (2×30.0 mL), dried over sodium sulfate, filtered and concentrated under reduced pressure to give a residue. The residue was purified by reversed phase-HPLC (0.1% formic acid condition) to afford methyl (S)-4-(trifluoromethyl)indoline-2-carboxylate (650 mg, 2.65 mmol, 41% yield) as a white solid. ¹H NMR (400 MHz, DMSO-d₆) δ=7.16 (br s, 1H), 6.81-6.78 (m, 1H), 6.59 (br s, 1H), 4.52 (ddd, J=1.6, 5.8, 10.6 Hz, 1H), 3.72-3.63 (m, 3H), 3.44 (br dd, J=11.2, 16.2 Hz, 1H), 3.22 (br dd, J=4.2, 17.2 Hz, 1H).

Step 4. To a solution of methyl (S)-4-(trifluoromethyl)indoline-2-carboxylate (300 mg, 1.22 mmol, 1.00 eq) in tetrahydrofuran (3.00 mL) was added lithium aluminium hydride (92.9 mg, 2.45 mmol, 2.00 eq) at 0° C. under nitrogen atmosphere, the mixture was stirred at 25° C. for 2 h. The reaction mixture was quenched with sodium sulfate decahydrate (50.0 mg) and diluted with ethyl acetate, filtered to give filtrate, then was concentrated under reduced pressure to afford (S)-(4-(trifluoromethyl)indolin-2-yl)methanol (550 mg, crude) as a yellow solid. MS (ESI) m/z 218.1 [M+H]⁺

Step 5. To a solution of (S)-(4-(trifluoromethyl)indolin-2-yl)methanol (200 mg, 921 mol, 1.00 eq) in dichloromethane (1.00 mL) was added di-tert-butyldicarbonate (221 mg, 1.01 mmol, 233 μL, 1.10 eq), the mixture was stirred at 50° C. for 12 h. The reaction mixture was diluted with water (30.0 mL) and exacted with ethyl acetate (3×50.0 mL). The organic phase was separated, washed with brine (2×50.0 mL), dried over sodium sulfate, filtered and concentrated under reduced pressure to give a residue. The residue was purified by column chromatography (SiO2, Petroleum ether/Ethyl acetate=5/1 to 1/1) to afford tert-butyl (S)-2-(hydroxymethyl)-4-(trifluoromethyl)indoline-1-carboxylate (100 mg, 315 mol, 34% yield) as yellow oil. ¹H NMR (400 MHz, DMSO-d₆) δ=7.98-7.73 (m, 1H), 7.34 (br d, J=8.4 Hz, 1H), 7.25-7.21 (m, 1H), 4.95 (t, J=5.6 Hz, 1H), 4.48-4.39 (m, 1H), 3.54 (br dd, J=3.2, 5.4 Hz, 1H), 3.51-3.46 (m, 1H), 3.43-3.36 (m, 1H), 3.15 (br d, J=17.4 Hz, 1H), 1.52 (s, 9H). MS (ESI) m/z 218.2 [M+H−100]⁺

Step 6. To a solution of tert-butyl (S)-2-(hydroxymethyl)-4-(trifluoromethyl)indoline-1-carboxylate (100 mg, 315 mol, 1.00 eq) in dimethylformamide (1.00 mL) was added sodium hydride (25.2 mg, 630 mol, 60% purity, 2.00 eq) and iodomethane (224 mg, 1.58 mmol, 98.1 μL, 5.00 eq), the mixture was stirred at 25° C. for 2 h. The reaction mixture was quenched with saturated ammonium chloride (2.00 mL), then diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by column chromatography (SiO2, Petroleum ether/Ethyl acetate=10/1 to 3/1) to afford tert-butyl (S)-2-(methoxymethyl)-4-(trifluoromethyl)indoline-1-carboxylate (100 mg, 302 mol, 96% yield) as a yellow solid. ¹H NMR (400 MHz, DMSO-d₆) δ=8.00-7.73 (m, 1H), 7.37 (t, J=8.0 Hz, 1H), 7.25 (d, J=7.8 Hz, 1H), 4.63-4.55 (m, 1H), 3.50-3.46 (m, 2H), 3.46-3.39 (m, 1H), 3.25 (s, 3H), 3.05 (br d, J=17.6 Hz, 1H), 1.53 (s, 9H). (H NMR comes from pilot run) MS (ESI) m/z 232.2 [M+H]⁺

Step 7. A solution of tert-butyl (S)-2-(methoxymethyl)-4-(trifluoromethyl)indoline-1-carboxylate (70.0 mg, 211 mol, 1.00 eq) in trifluoroacetic acid (0.200 mL) and dichloromethane (1.00 mL), the mixture was stirred at 25° C. for 1 h. The reaction mixture was concentrated under reduced pressure to afford (S)-2-(methoxymethyl)-4-(trifluoromethyl)indoline (50.0 mg, crude) as yellow oil.

Step 8. To a solution of (S)-2-(methoxymethyl)-4-(trifluoromethyl)indoline (50.0 mg, 216.25 mol, 1.00 eq) and phenyl (2-(2,6-dioxopiperidin-3-yl)-1-oxoisoindolin-5-yl)carbamate (123 mg, 324 mol, 1.50 eq) in dimethylformamide (1.00 mL) was added triethylamine (65.7 mg, 649 mol, 90.3 μL, 3.00 eq), the mixture was stirred at 50° C. for 12 h. The reaction mixture was added formic acid (0.200 mL) and filtered to give filtrate. The filtrate was purified by Prep-HPLC (column: Phenomenex luna C18 150*25 mm*10 um; mobile phase: [water (FA)-ACN]; B %: 42%-72%, 9 min) to afford (2S)—N-(2-(2,6-dioxopiperidin-3-yl)-1-oxoisoindolin-5-yl)-2-(methoxymethyl)-4-(trifluoromethyl)indoline-1-carboxamide (22.61 mg, 39.4 mol, 18% yield, 99% purity) as a white solid. ¹H NMR (400 MHz, DMSO-d₆) δ=10.99 (s, 1H), 9.26 (s, 1H), 8.06 (d, J=8.2 Hz, 1H), 7.84 (s, 1H), 7.68 (d, J=8.2 Hz, 1H), 7.57 (dd, J=1.4, 8.2 Hz, 1H), 7.42-7.36 (m, 1H), 7.27 (d, J=7.8 Hz, 1H), 5.13-5.02 (m, 2H), 4.51-4.41 (m, 1H), 4.34-4.27 (m, 1H), 3.58-3.51 (m, 3H), 3.34 (br s, 3H), 3.02 (br d, J=17.0 Hz, 1H), 2.97-2.86 (m, 1H), 2.65-2.59 (m, 1H), 2.40 (br dd, J=4.2, 13.2 Hz, 1H), 2.06-1.97 (m, 1H). MS (ESI) m/z 517.2 [M+H]⁺

### Example 51. Synthesis of Compound 307

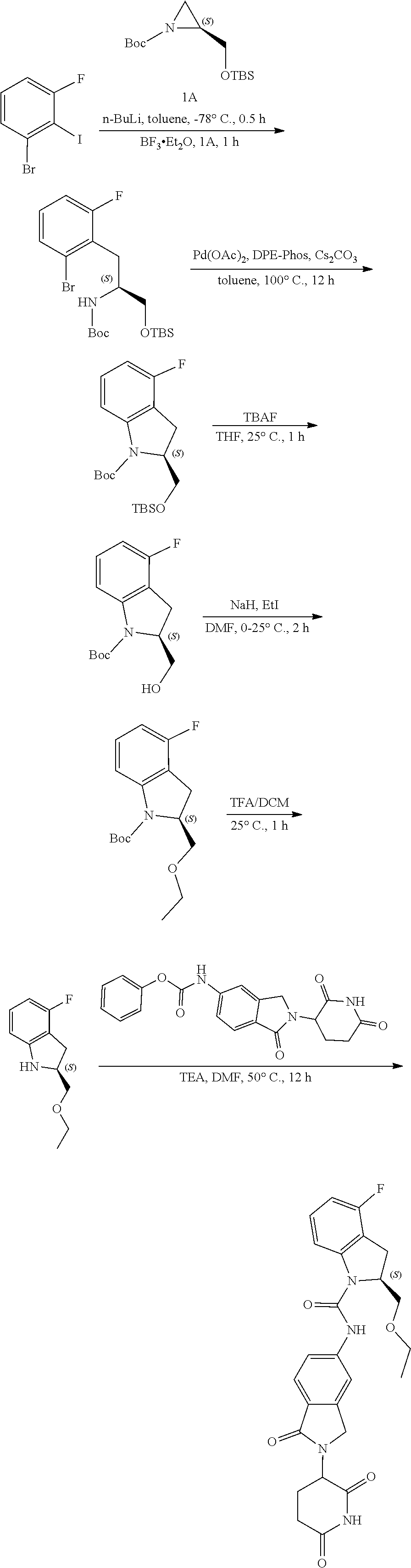

Step 1. To a solution of 1-bromo-3-fluoro-2-iodo-benzene (300 mg, 997 mol, 1.50 eq) in toluene (3.00 mL) was added n-butyllithium (2.50 M, 265 μL, 1.00 eq) at −78° C., the mixture was stirred at −78° C. for 0.5 h, then boron trifluoride diethyl etherate (113 mg, 797 mol, 98.0 μL, 1.20 eq) and tert-butyl (S)-(1-((tert-butyldimethylsilyl)oxy)-3-hydroxypropan-2-yl)carbamate (191 mg, 664 mol, 1.00 eq) in toluene. (0.500 mL) was added, the mixture was stirred at −78° C. for another 1 h. The reaction mixture was quenched with methanol (2.00 mL) and warmed to 25° C., the mixture was poured into water (80 mL) and extracted with ethyl acetate (3×40 mL). The combined organic phase was washed with brine (100 mL), dried over anhydrous sodium sulfate, filtered and concentrated in vacuum to give a residue. The residue was purified by reversed-phase (0.1% FA condition) to give (S)-tert-butyl (1-(2-bromo-6-fluorophenyl)-3-((tert-butyldimethylsilyl)oxy)propan-2-yl)carbamate (360 mg, crude) as colorless oil. ¹H NMR (400 MHz, DMSO-d₆) δ=7.43-7.37 (m, 1H), 7.19-7.14 (m, 2H), 6.48 (br d, J=9.4 Hz, 1H), 3.85-3.76 (m, 1H), 3.59-3.52 (m, 2H), 2.92 (br dd, J=3.7, 13.3 Hz, 1H), 2.79-2.72 (m, 1H), 1.23 (s, 9H), 0.83 (s, 9H), 0.00 (s, 6H).

Step 2. To a solution of (S)-tert-butyl (1-(2-bromo-6-fluorophenyl)-3-((tert-butyldimethylsilyl)oxy)propan-2-yl)carbamate (200 mg, 432 mol, 1.00 eq), [2-(2-diphenylphosphanylphenoxy)phenyl]-diphenyl-phosphane (46.6 mg, 86.5 mol, 0.200 eq) and cesium carbonate (281 mg, 864 mol, 2.00 eq) in toluene (4.00 mL) was added palladium acetate (19.4 mg, 86.5 mol, 0.200 eq), the mixture was stirred at 100° C. for 12 h. The reaction mixture was poured into water (80 mL) and extracted with ethyl acetate (3×40 mL). The combined organic phase was washed with brine (80 mL), dried over anhydrous sodium sulfate, filtered and concentrated in vacuum. The residue was purified by silica gel chromatography (Petroleum ether/Ethyl acetate=1/0 to 20/1) to afford (S)-tert-butyl 2-(((tert-butyldimethylsilyl)oxy)methyl)-4-fluoroindoline-1-carboxylate (150 mg, 393 mol, 90% yield) as colorless oil. ¹H NMR (400 MHz, DMSO-d₆) δ=7.67-7.30 (m, 1H), 7.22-7.11 (m, 1H), 6.75 (t, J=8.8 Hz, 1H), 4.55-4.43 (m, 1H), 3.81 (br s, 1H), 3.70 (br d, J=9.5 Hz, 1H), 3.31-3.24 (m, 1H), 3.04-2.90 (m, 1H), 1.53 (s, 9H), 0.69 (s, 9H), 0.00 (s, 3H), −0.10 (s, 3H).

Step 3. To a solution of (S)-tert-butyl 2-(((tert-butyldimethylsilyl)oxy)methyl)-4-fluoroindoline-1-carboxylate (130 mg, 340 mol, 1.00 eq) in tetrahydrofuran (3.00 mL) was added tetrabutylammonium fluoride (1.00 M, 408 μL, 1.20 eq), the mixture was stirred at 25° C. for 1 h. The reaction mixture was poured into water (80 mL) and extracted with ethyl acetate (3×40 mL). The combined organic phase was washed with brine (80 mL), dried over anhydrous sodium sulfate, filtered and concentrated in vacuum to give (S)-tert-butyl 4-fluoro-2-(hydroxymethyl)indoline-1-carboxylate (110 mg, crude) as colorless oil. MS (ESI) m/z 168.4 [M+H−100]⁺

Step 4. To a solution of (S)-tert-butyl 4-fluoro-2-(hydroxymethyl)indoline-1-carboxylate (100 mg, 374 mol, 1.00 eq) in dimethylformamide (2.00 mL) was added sodium hydride (29.9 mg, 748 mol, 60% purity, 2.00 eq), the mixture was stirred at 0° C. for 0.5 h, then iodoethane (116 mg, 748 mol, 60.0 μL, 2.00 eq) was added, the mixture was stirred at 25° C. for another 1.5 h. The reaction mixture was poured into water (40.0 mL) and extracted with ethyl acetate (3×20 mL). The combined organic phase was washed with brine (50 mL), dried over anhydrous sodium sulfate, filtered and concentrated in vacuum to give a residue. The residue was purified by silica gel chromatography (Petroleum ether/Ethyl acetate=1/0 to 20/1) to afford (S)-tert-butyl 2-(ethoxymethyl)-4-fluoroindoline-1-carboxylate (90.0 mg, 304 mol, 81% yield) as colorless oil. ¹H NMR (400 MHz, CDCl₃) δ=7.59-7.33 (m, 1H), 7.16-7.03 (m, 1H), 6.63 (t, J=8.5 Hz, 1H), 4.58 (br s, 1H), 3.63 (dd, J=3.9, 9.3 Hz, 1H), 3.56-3.43 (m, 2H), 3.31 (t, J=8.8 Hz, 1H), 3.23-3.13 (m, 1H), 3.12-3.03 (m, 1H), 1.54 (s, 9H), 1.17-1.13 (m, 3H).

Step 5. To a solution of (S)-tert-butyl 2-(ethoxymethyl)-4-fluoroindoline-1-carboxylate (60.0 mg, 203 mol, 1.00 eq) in dichloromethane (0.500 mL) was added trifluoroacetic acid (921 mg, 8.08 mmol, 600 μL, 39.7 eq), the mixture was stirred at 25° C. for 1 h. The reaction mixture was concentrated to give (S)-2-(ethoxymethyl)-4-fluoroindoline (40.0 mg, crude) as yellow oil.

Step 6. To a solution of (S)-2-(ethoxymethyl)-4-fluoroindoline (40.0 mg, 204 mol, 1.00 eq) and triethylamine (83.0 mg, 819 mol, 114 μL, 4.00 eq) in dimethylformamide (0.500 mL) was added phenyl (2-(2,6-dioxopiperidin-3-yl)-1-oxoisoindolin-5-yl)carbamate (62.0 mg, 163 mol, 0.800 eq), the mixture was stirred at 50° C. for 1 h. The reaction mixture was filtered to give a filtrate. The filtrate was purified by Prep-HPLC (column: Welch Xtimate C18 150*25 mm*5 um; mobile phase: [water (FA)-ACN]; gradient:30%-60% B over 9 min) and Prep-HPLC (column: Phenomenex luna C18 150*25 mm*10 um; mobile phase: [water (FA)-ACN]; gradient:32%-62% B over 8 min) to afford (2S)—N-(2-(2,6-dioxopiperidin-3-yl)-1-oxoisoindolin-5-yl)-2-(ethoxymethyl)-4-fluoroindoline-1-carboxamide (11.75 mg, 24.4 mol, 12% yield) as white solid. ¹H NMR (400 MHz, DMSO-d₆) δ=10.98 (br s, 1H), 9.24 (s, 1H), 7.83 (s, 1H), 7.67 (d, J=8.3 Hz, 1H), 7.60 (d, J=8.0 Hz, 1H), 7.53 (br d, J=8.4 Hz, 1H), 7.28-7.14 (m, 1H), 6.80 (t, J=8.5 Hz, 1H), 5.13-5.06 (m, 1H), 5.06-4.98 (m, 1H), 4.49-4.39 (m, 1H), 4.36-4.27 (m, 1H), 3.54 (br t, J=5.9 Hz, 4H), 3.41 (br s, 1H), 2.94-2.86 (m, 2H), 2.62 (br d, J=2.0 Hz, 1H), 2.41 (br dd, J=9.1, 13.3 Hz, 1H), 2.10-1.99 (m, 1H), 1.12 (t, J=6.9 Hz, 3H). MS (ESI) m/z 481.3 [M+H]⁺

### Example 52. Synthesis of Compound 328

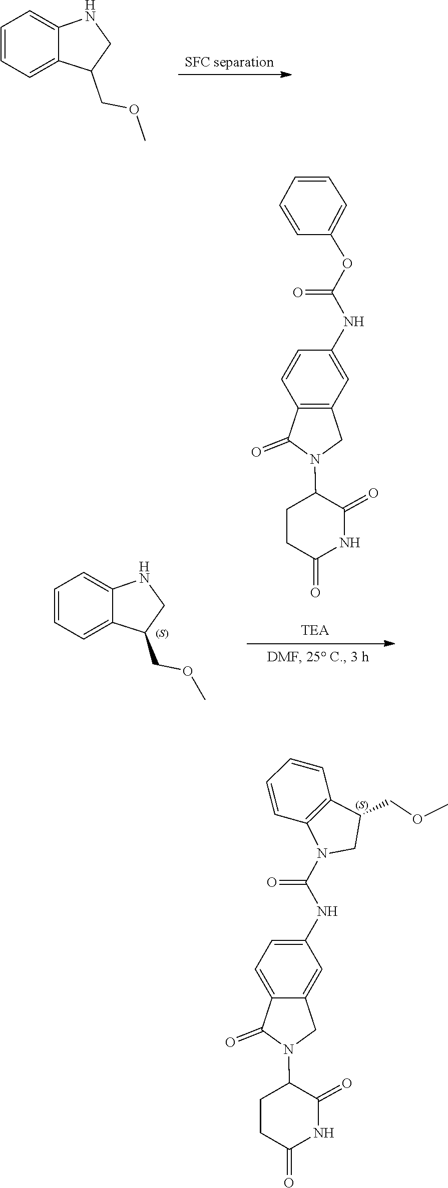

Step 1. The 3-(methoxymethyl)indoline (900 mg, 5.51 mmol, 93% purity) was purified by Superitical fluid chromatogram (column: DAICEL CHIRALPAK IA (250 mm*30 mm, 10 um); mobile phase: [Hexane-IPA (0.1% ammonium hydroxide)]; B %: 1%-1%, 15 min). The mixture was concentrated under reduced pressure to give a residue. Then the residue was re-purified by Superitical fluid chromatogram (column: DAICEL CHIRALPAK IA (250 mm*30 mm, 10 um); mobile phase: [IPA (0.1% IPAm)]; B %: 1%-1%, 12 min) The mixture was concentrated under reduced pressure to give a residue. Then the residue was re-purified by Superitical fluid chromatogram (column: DAICEL CHIRALPAK IA (250 mm*30 mm, 10 um); mobile phase: [IPA (0.1% IPAm)]; B %: 1%-1%, 12 min). The mixture was concentrated under reduced pressure to afford (S)-3-(methoxymethyl)indoline (200 mg, 1.23 mmol, 22% yield) as yellow oil. MS (ESI) m/z 164.1 [M+H]⁺

Step 2. To a solution of (S)-3-(methoxymethyl)indoline (200 mg, 1.23 mmol, 1.00 eq) and phenyl (2-(2,6-dioxopiperidin-3-yl)-1-oxoisoindolin-5-yl)carbamate (232 mg, 612 umol, 0.500 eq) in N,N-dimethyl formamide (2.00 mL) was added triethylamine (371 mg, 3.68 mmol, 511 μL, 3.00 eq). The mixture was stirred at 25° C. for 3 h. Then the mixture was adjusted pH=5 with formic acid (0.500 mL). The crude product was purified by Prep-HPLC (column: Phenomenex luna C18 150*25 mm*10 um; mobile phase: [water (formic acid)-acetonitrile]; B %: 22%-52%, 10 min) and lyophilized to afford (3S)—N-(2-(2,6-dioxopiperidin-3-yl)-1-oxoisoindolin-5-yl)-3-(methoxymethyl)indoline-1-carboxamide (15.4 mg, 32.7 umol, 2% yield, 95% purity) as a white solid.

¹H NMR (400 MHz, DMSO-d₆) δ=10.97 (br s, 1H), 8.88 (s, 1H), 7.96-7.87 (m, 2H), 7.70-7.61 (m, 2H), 7.27 (d, J=7.5 Hz, 1H), 7.18 (t, J=7.6 Hz, 1H), 6.94 (t, J=7.4 Hz, 1H), 5.09 (dd, J=5.1, 13.4 Hz, 1H), 4.48-4.39 (m, 1H), 4.33-4.23 (m, 2H), 4.02 (dd, J=5.8, 10.3 Hz, 1H), 3.77-3.67 (m, 1H), 3.63 (dd, J=5.0, 9.3 Hz, 1H), 3.45 (t, J=8.8 Hz, 1H), 3.32 (br s, 3H), 2.98-2.85 (m, 1H), 2.63-2.57 (m, 1H), 2.39 (dd, J=4.6, 12.9 Hz, 1H), 2.04-1.95 (m, 1H).

¹H NMR (400 MHz, DMSO+D₂O) δ=7.94-7.84 (m, 2H), 7.69-7.61 (m, 2H), 7.26 (d, J=7.4 Hz, 1H), 7.17 (t, J=8.1 Hz, 1H), 6.94 (t, J=7.6 Hz, 1H), 5.06 (dd, J=5.3, 13.3 Hz, 1H), 4.47-4.38 (m, 1H), 4.33-4.20 (m, 2H), 4.00 (dd, J=5.6, 10.1 Hz, 1H), 3.73-3.67 (m, 1H), 3.61 (dd, J=5.0, 9.3 Hz, 1H), 3.44 (br s, 1H), 3.31 (s, 3H), 2.96-2.83 (m, 1H), 2.60 (br dd, J=2.0, 15.0 Hz, 1H), 2.38 (br dd, J=4.5, 13.4 Hz, 1H), 2.05-1.96 (m, 1H). MS (ESI) m/z 449.3 [M+H]⁺

### Example 50. Synthesis of Compound 357

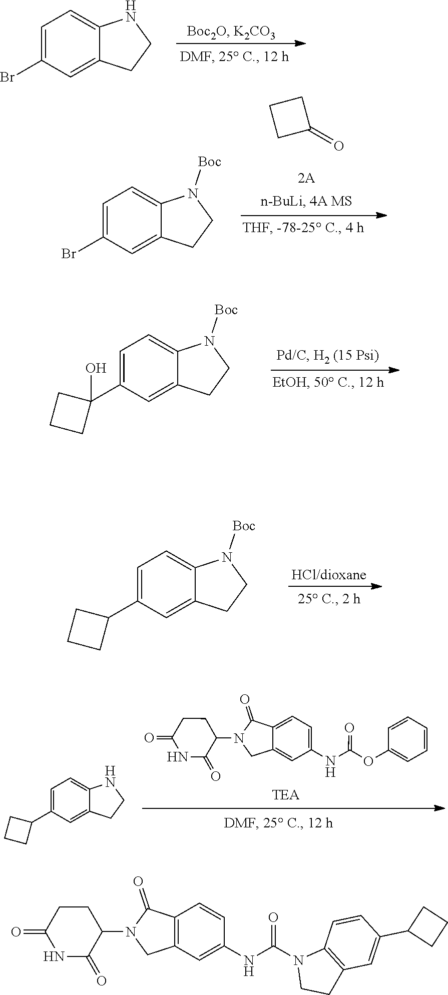

Step 1. To a solution of 5-bromoindoline (25.0 g, 126 mmol, 1.00 eq), potassium carbonate (87.23 g, 631.12 mmol, 5 eq) in dimethyformamide (250 mL) was added di-tert-butyldicarbonate (27.6 g, 126 mmol, 29.0 mL, 1.00 eq). The mixture was stirred at 25° C. for 12 h. The reaction mixture was added water (2 L) and filtered to give a solid. The filter cake was triturated with water (500 mL) and filtered to give tert-butyl 5-bromoindoline-1-carboxylate (30.0 g, 101 mmol, 80% yield) as a white solid. ¹H NMR (400 MHz, DMSO-d₆) δ=7.70-7.43 (m, 1H), 7.37 (s, 1H), 7.30 (br d, J=8.5 Hz, 1H), 3.90 (t, J=8.7 Hz, 2H), 3.06 (t, J=8.6 Hz, 2H), 1.49 (s, 9H).

Step 2. To a solution of tert-butyl 5-bromoindoline-1-carboxylate (2.00 g, 6.71 mmol, 1.00 eq), 4A molecular sieve (15.0 g, 6.71 mmol, 1.00 eq) in tetrahydrofuran (20.0 mL) was added n-butyllithium (2.50 M, 8.05 mL, 3.00 eq) dropwise at −78° C. under nitrogen. The mixture was stirred at −78° C. for 0.5 h, then the mixture was added cyclobutanone (940 mg, 13.4 mmol, 1.00 mL, 2.00 eq) dropwise at −78° C. under nitrogen. The mixture was stirred at 25° C. for 4 h under nitrogen. The reaction mixture was quenched by addition saturated ammonium chloride aqueous solution (30 mL), and extracted with ethyl acetate (3×30 mL), the combined organic phase was washed with brine (30 mL), dried with anhydrous sodium sulfate, filtered and concentrated in vacuum. The residue was purified by reversed-phase HPLC (column: spherical C18, 20-45 um, 100 Å, SW 120, mobile phase: [water (0.1% Formic Acid)-ACN) and lyophilized to give tert-butyl 5-(1-hydroxycyclobutyl)indoline-1-carboxylate (600 mg, 2.07 mmol, 31% yield) as a white solid.

¹H NMR (400 MHz, DMSO-d₆) δ=7.73-7.43 (m, 1H), 7.29 (s, 1H), 7.23 (dd, J=1.8, 8.3 Hz, 1H), 5.34 (s, 1H), 3.89 (t, J=8.6 Hz, 2H), 3.05 (t, J=8.6 Hz, 2H), 2.39-2.29 (m, 2H), 2.27-2.16 (m, 2H), 1.92-1.82 (m, 1H), 1.63-1.56 (m, 1H), 1.50 (s, 9H).

Step 3. To a solution of tert-butyl 5-(1-hydroxycyclobutyl)indoline-1-carboxylate (50.0 mg, 173 umol, 1.00 eq) in methanol (20.0 mL) was added palladium on carbon (200 mg, 10% purity) under nitrogen. The suspension was degassed under vacuum and purged with hydrogen several times. The mixture was stirred under hydrogen (15 Psi) at 50° C. for 12 h. The mixture was filtered. The filtrate was concentrated under reduced pressure to give tert-butyl 5-cyclobutylindoline-1-carboxylate (300 mg, crude) as yellow oil. ¹H NMR (400 MHz, DMSO-d₆) δ=7.69-7.43 (m, 1H), 7.06 (s, 1H), 6.97 (d, J=8.1 Hz, 1H), 3.87 (t, J=8.7 Hz, 2H), 3.45-3.40 (m, 1H), 3.02 (br t, J=8.6 Hz, 2H), 2.28-2.20 (m, 2H), 2.08-1.86 (m, 4H), 1.49 (s, 9H).

Step 4. A solution of tert-butyl 5-cyclobutylindoline-1-carboxylate (300 mg, 1.10 mmol, 1.00 eq) in hydrochloric acid (4 M in dioxane, 15.0 mL) was stirred at 25° C. for 2 h. The mixture was concentrated under reduced pressure to give 5-cyclobutylindoline (300 mg, crude) as brown oil. ¹H NMR (400 MHz, DMSO-d₆) δ=7.35-7.29 (m, 2H), 7.21 (d, J=8.3 Hz, 1H), 3.69 (t, J=7.8 Hz, 2H), 3.52 (br d, J=9.0 Hz, 1H), 3.19-3.15 (m, 2H), 2.34-2.25 (m, 2H), 2.11-2.02 (m, 2H), 2.01-1.87 (m, 2H). MS (ESI) m/z. 173.8 [M+H]⁺

Step 5. To a solution of 5-cyclobutylindoline (250 mg, 1.44 mmol, 1.00 eq), phenyl (2-(2,6-dioxopiperidin-3-yl)-1-oxoisoindolin-5-yl)carbamate (547 mg, 1.44 mmol, 1.00 eq) in dimethyformamide (2.00 mL) was added triethylamine (584 mg, 5.77 mmol, 803 μL, 4.00 eq). The mixture was stirred at 25° C. for 12 h. The mixture was added formic acid (0.2 mL) to adjusted pH=3-4. The mixture was added water (30 mL) and filtered. The filter cake was purified by prep-NPLC (column: Welch Ultimate XB—NH2 250*50*10 um; mobile phase: [Hexane-EtOH]; B %: 10%-60%, 30 min) and concentrated under reduced pressure. The residue was purified by reverse phase chromatography (column: spherical C18, 20-45 um, 100 Å, SW 40, mobile phase: [water (0.1% Formic Acid)-ACN) and lyophilized to give 5-cyclobutyl-N-(2-(2,6-dioxopiperidin-3-yl)-1-oxoisoindolin-5-yl)indoline-1-carboxamide (85.8 mg, 185 umol, 13% yield, 99% purity) as a white solid. ¹H NMR (400 MHz, DMSO-d₆) δ=10.97 (br s, 1H), 8.80 (s, 1H), 7.91 (s, 1H), 7.78 (d, J=8.3 Hz, 1H), 7.64 (s, 2H), 7.09 (s, 1H), 6.99 (br d, J=8.4 Hz, 1H), 5.08 (dd, J=5.1, 13.2 Hz, 1H), 4.49-4.37 (m, 1H), 4.34-4.25 (m, 1H), 4.15 (br t, J=8.6 Hz, 2H), 3.48-3.42 (m, 1H), 3.16 (br t, J=8.5 Hz, 2H), 2.96-2.86 (m, 1H), 2.60 (br d, J=16.0 Hz, 1H), 2.39 (br dd, J=4.6, 13.2 Hz, 1H), 2.30-2.21 (m, 2H), 2.09-1.91 (m, 4H), 1.85-1.73 (m, 1H). MS (ESI) m/z. 459.2 [M+H]⁺

### Example 53. Synthesis of Compound 355

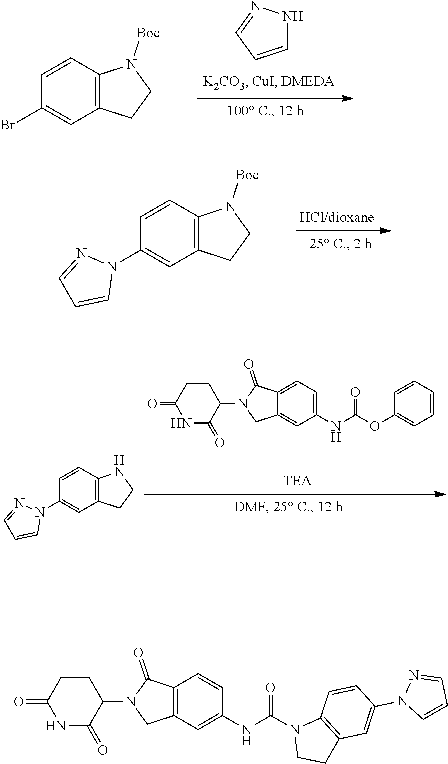 [SMILES: CC(C)(C)OC(=O)N1CCc2cc(Br)ccc21 ; c1cn[nH]c1 ; CC(C)(C)OC(=O)N1CCc2cc(-n3cccn3)ccc21 ; O=C1CCC(N2Cc3cc(NC(=O)Oc4ccccc4)ccc3C2=O)C(=O)N1 ; c1cnn(-c2ccc3c(c2)CCN3)c1 ; O=C1CCC(N2Cc3cc(NC(=O)N4CCc5cc(-n6cccn6)ccc54)ccc3C2=O)C(=O)N1]

Step 1. To a solution of tert-butyl 5-bromoindoline-1-carboxylate (1.00 g, 3.35 mmol, 1.10 eq) and 1H-pyrazole (208 mg, 3.05 mmol, 1.00 eq) in dioxane (10.0 mL) was added potassium carbonate (1.26 g, 9.15 mmol, 3.00 eq), N,N-dimethylethylenediamine (53.8 mg, 610 umol, 65.6 uL, 0.200 eq) and cuprous iodide (58.1 mg, 305 umol, 0.100 eq) under nitrogen. The mixture was stirred at 100° C. for 2 h. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by reversed-phase HPLC (column: spherical C18, 20-45 um, 100 Å, SW 120, mobile phase: [water (0.1% Formic Acid)-ACN) and lyophilized to give tert-butyl 5-(1H-pyrazol-1-yl)indoline-1-carboxylate (400 mg, 1.40 mmol, 45% yield) as a white solid. ¹H NMR (400 MHz, DMSO-d₆) δ=8.37 (d, J=2.4 Hz, 1H), 7.80-7.65 (m, 3H), 7.60 (dd, J=2.1, 8.6 Hz, 1H), 6.49 (t, J=2.1 Hz, 1H), 3.95 (t, J=8.7 Hz, 2H), 3.12 (t, J=8.7 Hz, 2H), 1.51 (s, 9H).

Step 2. A mixture of tert-butyl 5-(1H-pyrazol-1-yl)indoline-1-carboxylate (400 mg, 1.40 mmol, 1.00 eq) in hydrochloric acid/dioxane (4M, 10.0 mL) was stirred at 25° C. for 2 h. The mixture was concentrated in vacuum to give 5-(1H-pyrazol-1-yl)indoline (200 mg, crude) as a yellow solid. MS (ESI) m/z. 186.4 [M+H]⁺

Step 3. To a mixture of 5-(1H-pyrazol-1-yl)indoline (73.2 mg, 395 umol, 1.50 eq) and phenyl (2-(2,6-dioxopiperidin-3-yl)-1-oxoisoindolin-5-yl)carbamate (100 mg, 264 umol, 1.00 eq) in dimethyformamide (1.00 mL) was added triethylamine (80.0 mg, 791 umol, 110 μL, 3.00 eq). The mixture was stirred at 25° C. for 12 h. The mixture was added water (10 mL) and filtered. The filter cake was dissolved in dimethyformamide (15 mL), then the mixture was added water (50 mL). The solution was filtered again and the filter cake was added water (10 mL). The solution was lyophilized to give N-(2-(2,6-dioxopiperidin-3-yl)-1-oxoisoindolin-5-yl)-5-(1H-pyrazol-1-yl)indoline-1-carboxamide (100.57 mg, 212 umol, 80% yield, 99% purity) as an off-white solid. ¹H NMR (400 MHz, DMSO-d₆) δ=10.98 (s, 1H), 8.89 (s, 1H), 8.39 (d, J=2.4 Hz, 1H), 8.00-7.89 (m, 2H), 7.77-7.49 (m, 5H), 6.50 (t, J=2.1 Hz, 1H), 5.10 (dd, J=5.1, 13.3 Hz, 1H), 4.49-4.40 (m, 1H), 4.34-4.19 (m, 3H), 3.27 (br t, J=8.6 Hz, 2H), 2.95-2.86 (m, 1H), 2.60 (br d, J=16.0 Hz, 1H), 2.45-2.33 (m, 1H), 2.06-1.95 (m, 1H). MS (ESI) m/z. 471.2 [M+H]⁺

### Example 54. Synthesis of Compound 351

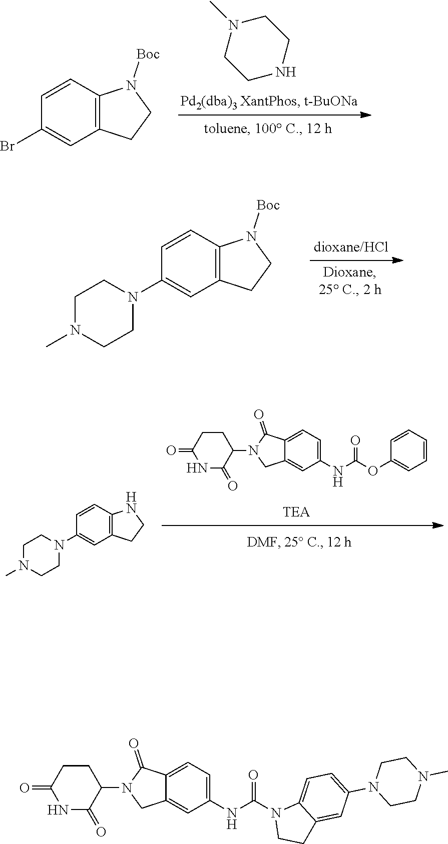

Step 1. To a solution of tert-butyl 5-bromoindoline-1-carboxylate (1.00 g, 3.35 mmol, 1.00 eq) and 1-methylpiperazine (403 mg, 4.02 mmol, 446 μL, 1.20 eq) in toluene (10.0 mL) was added 4,5-bis(diphenylphosphino)-9,9-dimethylxanthene (194 mg, 335 umol, 0.100 eq) and sodium tert-butoxide (483 mg, 5.03 mmol, 1.50 eq), tris(dibenzylideneacetone)dipalladium(0) (307 mg, 335 umol, 0.100 eq) under nitrogen atmosphere. The mixture was stirred at 100° C. for 12 h. After being cooled to room temperature, the mixture was diluted with water (200 mL) and extracted with ethyl acetate (3×100 mL). The organic layers were collected and was washed with brine (50.0 mL), dried over anhydrous sodium sulfate and evaporated to dryness. The crude product was purified by column chromatography on silica gel eluted with petroleum ether/ethyl acetate=50/1 to 0/1 and further purified by reversed-phase HPLC (column: spherical C18, 20-45 um, 100 Å, SW 120, mobile phase: [water (0.1% Formic Acid)-ACN]; B %: 15%-45%, 30 min) to afford tert-butyl 5-(4-methylpiperazin-1-yl)indoline-1-carboxylate (300 mg, 945 umol, 28% yield) as a black solid. ¹H NMR (400 MHz, DMSO-d₆) δ=7.27-7.16 (m, 1H), 6.83 (br s, 1H), 6.70 (br d, J=8.6 Hz, 1H), 3.84 (br t, J=8.6 Hz, 2H), 3.16-3.08 (m, 2H), 3.05-3.00 (m, 4H), 2.48 (br s, 4H), 2.24 (s, 3H), 1.48 (br s, 9H). MS (ESI) m/z 317.9 [M+H]⁺

Step 2. To a solution of tert-butyl 5-(4-methylpiperazin-1-yl)indoline-1-carboxylate (100 mg, 315 umol, 1.00 eq) in dioxane (2.00 mL) was added hydrochloric acid/dioxane (4.00 M, 2.00 mL). The mixture was stirred at 25° C. for 2 h. The mixture was concentrated under reduced pressure to afford 5-(4-methylpiperazin-1-yl)indoline (60 mg, crude) as a red solid.

Step 3. To a solution of 5-(4-methylpiperazin-1-yl)indoline (30.0 mg, 138 umol, 1.00 eq) and phenyl (2-(2,6-dioxopiperidin-3-yl)-1-oxoisoindolin-5-yl)carbamate (52.4 mg, 138 umol, 1.00 eq) in N,N-dimethyl formamide (1.00 mL) was added triethylamine (41.9 mg, 414 umol, 57.7 uL, 3.00 eq). The mixture was stirred at 25° C. for 12 h. The mixture was filtered. The filtrate was purified by prep-HPLC (column: Welch Ultimate C18 150*25 mm*5 um; mobile phase: [water (FA)-ACN]; B %: 1%-31%, 10 min) and lyophilized to afford N-(2-(2,6-dioxopiperidin-3-yl)-1-oxoisoindolin-5-yl)-5-(4-methylpiperazin-1-yl)indoline-1-carboxamide (9.82 mg, 18.6 umol, 13% yield, 95% purity) as a brown solid. ¹H NMR (400 MHz, DMSO-d₆) δ=10.97 (br s, 1H), 8.73 (s, 1H), 8.21 (s, 1H), 7.91 (s, 1H), 7.72 (d, J=8.9 Hz, 1H), 7.63 (s, 1H), 6.86 (s, 1H), 6.72 (dd, J=2.3, 8.9 Hz, 1H), 5.08 (dd, J=5.0, 13.4 Hz, 1H), 4.46-4.39 (m, 1H), 4.32-4.24 (m, 1H), 4.12 (t, J=8.5 Hz, 2H), 3.13 (br t, J=8.5 Hz, 2H), 3.06-3.02 (m, 4H), 2.94-2.87 (m, 1H), 2.62-2.57 (m, 1H), 2.46-2.43 (m, 4H), 2.39 (br dd, J=4.6, 13.2 Hz, 1H), 2.22 (s, 3H), 2.03-1.96 (m, 1H). MS (ESI) m/z 503.0 [M+H]⁺

### Example 55. Synthesis of Compound 352

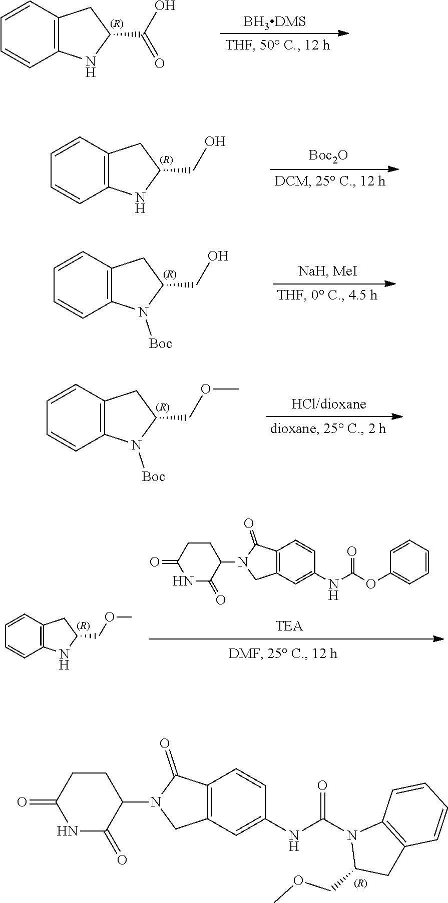

Step 1. To a solution of (R)-indolin-2-ylmethanol (2.00 g, 12.3 mmol, 1.00 eq) in tetrahydrofuran (20.0 mL) was added borane dimethyl sulfide complex (10.0 M, 2.45 mL, 2.00 eq) at 0° C. The mixture was stirred at 50° C. for 12 h. After being cooled to room temperature, the mixture was added methyl alcohol (10.0 mL) to give solution. The solution was concentrated under reduced pressure to give a residue. The crude product was purified by column chromatography on silica gel eluted with petroleum ether/ethyl acetate=50/1 to 1/1 to afford (R)-indolin-2-ylmethanol (1.00 g, 6.70 mmol, 55% yield) as a black solid. ¹H NMR (400 MHz, DMSO-d₆) δ=6.96 (br d, J=7.1 Hz, 1H), 6.87 (br t, J=7.6 Hz, 1H), 6.53-6.41 (m, 2H), 5.51 (br s, 1H), 4.73 (t, J=5.1 Hz, 1H), 3.82-3.70 (m, 1H), 3.41-3.36 (m, 1H), 2.97 (br dd, J=9.2, 15.8 Hz, 1H), 2.63 (br dd, J=6.7, 15.8 Hz, 1H).

Step 2. To a solution of (R)-indolin-2-ylmethanol (500 mg, 3.35 mmol, 1.00 eq) in dichloromethane (5.00 mL) was added di-tert-butyl dicarbonate (805 mg, 3.69 mmol, 847 μL, 1.10 eq). The mixture was stirred at 25° C. for 12 h. The mixture was diluted with water (30 mL) and extracted with ethyl acetate (3×30 mL). The organic layers were collected and washed with brine (20.0 mL), dried over anhydrous sodium sulfate and evaporated to dryness. The crude product was purified by column chromatography on silica gel eluted with petroleum ether/ethyl acetate=50/1 to 5/1 to afford (R)-tert-butyl 2-(methoxymethyl)indoline-1-carboxylate (800 mg, 3.21 mmol, 96% yield) as yellow oil. ¹H NMR (400 MHz, DMSO-d₆) δ=7.71-7.42 (m, 1H), 7.17 (d, J=7.4 Hz, 1H), 7.11 (t, J=7.7 Hz, 1H), 6.94-6.85 (m, 1H), 4.87 (t, J=5.6 Hz, 1H), 4.33 (dt, J=3.4, 6.5 Hz, 1H), 3.69-3.47 (m, 1H), 3.39-3.33 (m, 1H), 3.20 (dd, J=9.9, 16.4 Hz, 1H), 3.03-2.93 (m, 1H), 1.50 (s, 9H).

Step 3. To a solution of (R)-tert-butyl 2-(hydroxymethyl)indoline-1-carboxylate (800 mg, 3.21 mmol, 1.00 eq) in tetrahydrofuran (5.00 mL) was added sodium hydride (141 mg, 3.53 mmol, 60% purity, 1.10 eq) at 0° C. under nitrogen. The mixture was stirred at 0° C. for 0.5 h, then it was added methyl iodide (1.82 g, 12.8 mmol, 799 μL, 4.00 eq) at 0° C. under nitrogen. The mixture was stirred at 25° C. for 4 h. The mixture was quenched with saturated ammonium chloride aqueous solution (50 mL), then extracted with ethyl acetate (3×50 mL). The organic layers were collected and was washed with brine (30.0 mL), dried over anhydrous sodium sulfate and evaporated to dryness. The crude product was purified by column chromatography on silica gel eluted with petroleum ether/ethyl acetate=50/1 to 10/1 to afford (R)-tert-butyl 2-(methoxymethyl)indoline-1-carboxylate (200 mg, 759 umol, 24% yield) as yellow oil. ¹H NMR (400 MHz, DMSO-d₆) δ=7.64-7.46 (m, 1H), 7.17 (d, J=7.3 Hz, 1H), 7.12 (t, J=7.8 Hz, 1H), 6.92 (t, J=7.4 Hz, 1H), 4.48 (tt, J=3.3, 6.6 Hz, 1H), 3.47 (dd, J=3.6, 9.6 Hz, 1H), 3.38-3.33 (m, 1H), 3.27 (br d, J=10.8 Hz, 1H), 3.25 (s, 3H), 2.89 (dd, J=2.1, 16.4 Hz, 1H), 1.51 (s, 9H).

Step 4. A solution of (R)-tert-butyl 2-(methoxymethyl)indoline-1-carboxylate (200 mg, 759 umol, 1.00 eq) in hydrochloric acid (4 M in dioxane, 5.00 mL) was stirred at 25° C. for 2 h. The mixture was concentrated under reduced pressure to afford (R)-2-(methoxymethyl)indoline (200 mg, crude) as red oil.

Step 5. To a solution of (R)-2-(methoxymethyl)indoline (200 mg, 1.23 mmol, 1.00 eq), phenyl (2-(2,6-dioxopiperidin-3-yl)-1-oxoisoindolin-5-yl)carbamate (465 mg, 1.23 mmol, 1.00 eq) in N,N-dimethyl formamide (2.00 mL) was added triethylamine (372 mg, 3.68 mmol, 512 μL, 3.00 eq). The mixture was stirred at 25° C. for 12 h. The mixture was added formic acid (0.3 mL) to adjusted pH=3-4. The mixture was purified by reverse phase chromatography (column: spherical C18, 20-45 um, 100 Å, SW 40, mobile phase: [water (0.1% Formic Acid)-ACN) and lyophilized to give (2R)—N-(2-(2,6-dioxopiperidin-3-yl)-1-oxoisoindolin-5-yl)-2-(methoxymethyl)indoline-1-carboxamide (21.8 mg, 48.2 umol, 4% yield, 99% purity) as a white solid.

¹H NMR (400 MHz, DMSO-d₆) δ=11.10-10.78 (m, 1H), 9.16 (s, 1H), 7.81 (s, 1H), 7.75 (d, J=8.0 Hz, 1H), 7.65 (d, J=8.3 Hz, 1H), 7.53 (d, J=8.8 Hz, 1H), 7.22 (d, J=7.4 Hz, 1H), 7.15 (t, J=7.7 Hz, 1H), 7.00-6.87 (m, 1H), 5.08 (dd, J=5.1, 13.3 Hz, 1H), 5.00-4.86 (m, 1H), 4.50-4.40 (m, 1H), 4.35-4.25 (m, 1H), 3.53-3.44 (m, 4H), 3.40 (br s, 2H), 2.96-2.80 (m, 2H), 2.60 (td, J=2.0, 15.6 Hz, 1H), 2.42-2.30 (m, 1H), 2.04-1.91 (m, 1H)

¹H NMR (400 MHz, CDCl₃) δ=9.22 (s, 1H), 8.01-7.93 (m, 2H), 7.83-7.72 (m, 2H), 7.25-7.21 (m, 1H), 7.18 (d, J=7.4 Hz, 1H), 7.15-7.04 (m, 1H), 7.03-6.96 (m, 1H), 5.30-5.12 (m, 1H), 4.76-4.62 (m, 1H), 4.54-4.41 (m, 1H), 4.38-4.27 (m, 1H), 3.62 (d, J=6.1 Hz, 2H), 3.56 (s, 3H), 3.55-3.49 (m, 1H), 3.00-2.88 (m, 1H), 2.87-2.77 (m, 1H), 2.62 (br d, J=15.8 Hz, 1H), 2.37 (ddd, J=5.2, 7.7, 13.0 Hz, 1H), 2.28-2.18 (m, 1H). MS (ESI) m/z 449.1 [M+H]⁺

### Example 56. Synthesis of Compound 363

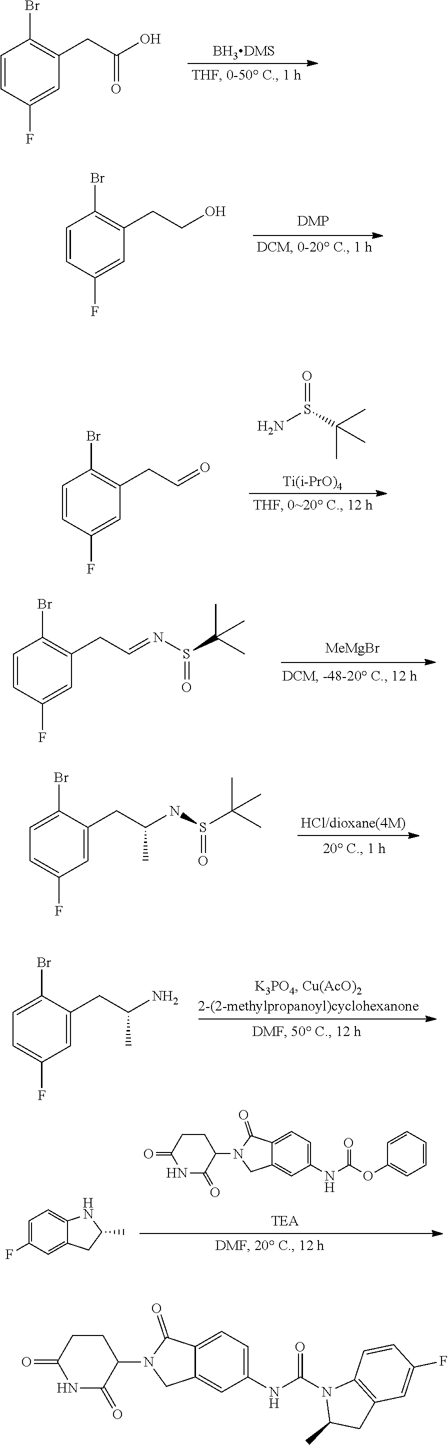

Step 1. To a solution of 2-(2-bromo-5-fluorophenyl)acetic acid (10.0 g, 42.9 mmol, 1.00 eq) in tetrahydrofuran (100 mL) was added borane dimethyl sulfide complex (10 M, 6.44 mL, 1.50 eq) at 0° C. The mixture was stirred at 50° C. for 1 h under nitrogen. The reaction mixture was quenched with methanol (10 mL) and concentrated under reduced pressure to give a residue. The residue was added methanol (100 mL) and stirred at 80° C. for 0.5 h, then it was concentrated under reduced pressure to give 2-(2-bromo-5-fluorophenyl)ethanol (9.00 g, 41.0 mmol, 95% yield) as yellow oil. ¹H NMR (400 MHz, DMSO-d₆) δ=7.61 (dd, J=5.5, 8.8 Hz, 1H), 7.24 (dd, J=3.0, 9.8 Hz, 1H), 7.04 (dt, J=3.1, 8.5 Hz, 1H), 4.77 (t, J=5.3 Hz, 1H), 3.64-3.59 (m, 2H), 2.84 (t, J=6.9 Hz, 2H).

Step 2. To a solution of 2-(2-bromo-5-fluorophenyl)ethanol (4.50 g, 20.5 mmol, 1.00 eq) in dichloromethane (100 mL) was added Dess-Martin periodinane (13.0 g, 30.8 mmol, 9.54 mL, 1.50 eq) at 0° C. The mixture was stirred at 20° C. for 1 h. The mixture was filtered. The filter liquor was diluted with water (100 mL) and extracted with ethyl acetate (2×100 mL). The organic phase was separated, washed with brine (10 mL), dried over sodium sulfate, filtered and concentrated under reduced pressure to give a residue. The residue was purified by column chromatography (SiO₂, Petroleum ether/Ethyl acetate=10/1 to 0/1) to give 2-(2-bromo-5-fluorophenyl)acetaldehyde (8.80 g, 40.5 mmol, 98% yield) as yellow oil. ¹H NMR (400 MHz, DMSO-d₆) δ=9.70 (s, 1H), 7.68 (dd, J=5.5, 9.0 Hz, 1H), 7.31 (dd, J=3.0, 9.5 Hz, 1H), 7.14 (dt, J=3.1, 8.5 Hz, 1H), 3.97 (s, 2H).

Step 3. To a solution of 2-(2-bromo-5-fluorophenyl)acetaldehyde (6.80 g, 31.3 mmol, 1.00 eq) in tetrahydrofuran (70.0 mL) was added (R)-2-methylpropane-2-sulfinamide (4.18 g, 34.4 mmol, 1.10 eq) and titanium(IV) isopropoxide (13.3 g, 47.0 mmol, 13.8 mL, 1.50 eq) at 0° C. The mixture was stirred at 20° C. for 2 h under nitrogen. The reaction mixture was diluted with water (100 mL) and extracted with ethyl acetate (2×200 mL). The organic phase was separated, washed with brine (50 mL), dried over sodium sulfate, filtered and concentrated under reduced pressure. The residue was and purified by column chromatography (SiO₂, Petroleum ether/Ethyl acetate=50/1 to 0/1) to give (R,E)-N-(2-(2-bromo-5-fluorophenyl)ethylidene)-2-methylpropane-2-sulfinamide (3.00 g, 9.37 mmol, 29% yield) as yellow oil. ¹H NMR (400 MHz, DMSO-d₆) δ=8.01 (t, J=3.8 Hz, 1H), 7.67 (dd, J=5.4, 8.8 Hz, 1H), 7.36 (dd, J=3.1, 9.5 Hz, 1H), 7.13 (dt, J=3.1, 8.6 Hz, 1H), 4.02 (d, J=3.6 Hz, 2H), 1.05 (s, 9H).

Step 4. To a solution of (R,E)-N-(2-(2-bromo-5-fluorophenyl)ethylidene)-2-methylpropane-2-sulfinamide (1.50 g, 4.68 mmol, 1.00 eq) in dichloromethane (30 mL) was added bromo(methyl)magnesium (3.00 M, 7.81 mL, 5.00 eq) under nitrogen at −48° C. The mixture was stirred at 20° C. for 12 h. The mixture was quenched by addition saturated ammonium chloride solution (100 mL) at 0° C., then extracted with ethyl acetate (2×50 mL). The combined organic layers were washed with brine (20 mL), dried over sodium sulfate, filtered and concentrated under reduced pressure. The residue and purified by reversed-phase HPLC (column: spherical C18, 20-45 um, 100 Å, SW 120, mobile phase: [water (0.1% Formic Acid)-ACN]) to give (R)—N—((R)-1-(2-bromo-5-fluorophenyl)propan-2-yl)-2-methylpropane-2-sulfinamide (0.600 g, 1.78 mmol, 38% yield) as a yellow solid. ¹H NMR (400 MHz, DMSO-d₆) δ=7.59 (dd, J=5.6, 8.8 Hz, 1H), 7.21 (dd, J=3.1, 9.8 Hz, 1H), 7.02 (dt, J=3.2, 8.5 Hz, 1H), 4.99 (d, J=9.1 Hz, 1H), 3.55-3.42 (m, 1H), 2.88-2.74 (m, 2H), 1.28 (d, J=6.5 Hz, 3H), 0.94 (s, 9H).

Step 5. A mixture of (R)—N—((R)-1-(2-bromo-5-fluorophenyl)propan-2-yl)-2-methylpropane-2-sulfinamide (1.10 g, 3.27 mmol, 1.00 eq) in hydrochloric acid/dioxane (4.00 M, 5.00 mL, 6.11 eq) was stirred at 20° C. for 1 h. The reaction mixture was concentrated under reduced pressure. The residue was purified by prep-HPLC (column: Welch Ultimate C18 150*25 mm*5 um; mobile phase: [water (FA)-ACN]; B %: 0%-32%, 10 min) and lyophilized to give (R)-1-(2-bromo-5-fluorophenyl)propan-2-amine (0.700 g, 3.02 mmol, 92% yield) as a white solid. ¹H NMR (400 MHz, CDCl₃) δ=7.54 (dd, J=5.4, 8.8 Hz, 1H), 7.15 (dd, J=2.9, 8.9 Hz, 1H), 6.90 (dt, J=2.9, 8.3 Hz, 1H), 3.82-3.69 (m, 1H), 3.24 (dd, J=7.1, 13.7 Hz, 1H), 3.08 (dd, J=7.3, 13.8 Hz, 1H), 1.48 (d, J=6.5 Hz, 3H).

Step 6. To a solution of (R)-1-(2-bromo-5-fluorophenyl)propan-2-amine (50.0 mg, 215 umol, 1.00 eq) in dimethyl formamide (1.00 mL) was added 2-(2-methylpropanoyl)cyclohexanone (7.25 mg, 43.0 umol, 0.200 eq), potassium phosphate (91.4 mg, 430 umol, 2.00 eq) and copper(II)acetate (1.96 mg, 10.7 umol, 0.0500 eq). The mixture was stirred at 50° C. for 12 h under nitrogen. The mixture was filtered to give a (R)-5-fluoro-2-methylindoline (1.00 g, crude) in dimethyl formamide (1.00 mL) as a liquid.

Step 7. To a solution of (R)-5-fluoro-2-methylindoline (1.00 g, crude) in dimethyl formamide (1.00 mL) was added triethylamine (60.2 mg, 595 umol, 82.8 uL, 0.0900 eq) and phenyl (2-(2,6-dioxopiperidin-3-yl)-1-oxoisoindolin-5-yl)carbamate (75.2 mg, 198 umol, 0.0300 eq). The mixture was stirred at 20° C. for 12 h. The mixture was filtered to give a filter liquor which was purified by prep-HPLC (column: Phenomenex luna C18 150*25 mm*10 um; mobile phase: [water (FA)-ACN]; B %: 27%-57%, 10 min) and lyophilized to give (2R)—N-(2-(2,6-dioxopiperidin-3-yl)-1-oxoisoindolin-5-yl)-5-fluoro-2-methylindoline-1-carboxamide (6.39 mg, 14.64 umol, 0.08% yield) as a white solid. ¹H NMR (400 MHz, DMSO-d₆) δ=10.98 (br s, 1H), 8.94 (s, 1H), 7.89 (s, 1H), 7.86 (dd, J=4.9, 8.9 Hz, 1H), 7.65 (s, 2H), 7.14-7.08 (m, 1H), 6.97 (dt, J=2.7, 9.1 Hz, 1H), 5.09 (dd, J=4.9, 13.4 Hz, 1H), 4.98-4.88 (m, 1H), 4.46-4.39 (m, 1H), 4.34-4.22 (m, 1H), 3.41 (br dd, J=9.2, 16.2 Hz, 1H), 2.96-2.87 (m, 1H), 2.72 (br d, J=16.1 Hz, 1H), 2.62-2.57 (m, 1H), 2.45-2.36 (m, 1H), 2.04-1.96 (m, 1H), 1.23 (d, J=6.1 Hz, 3H).

### Example 57. Synthesis of Compound 354

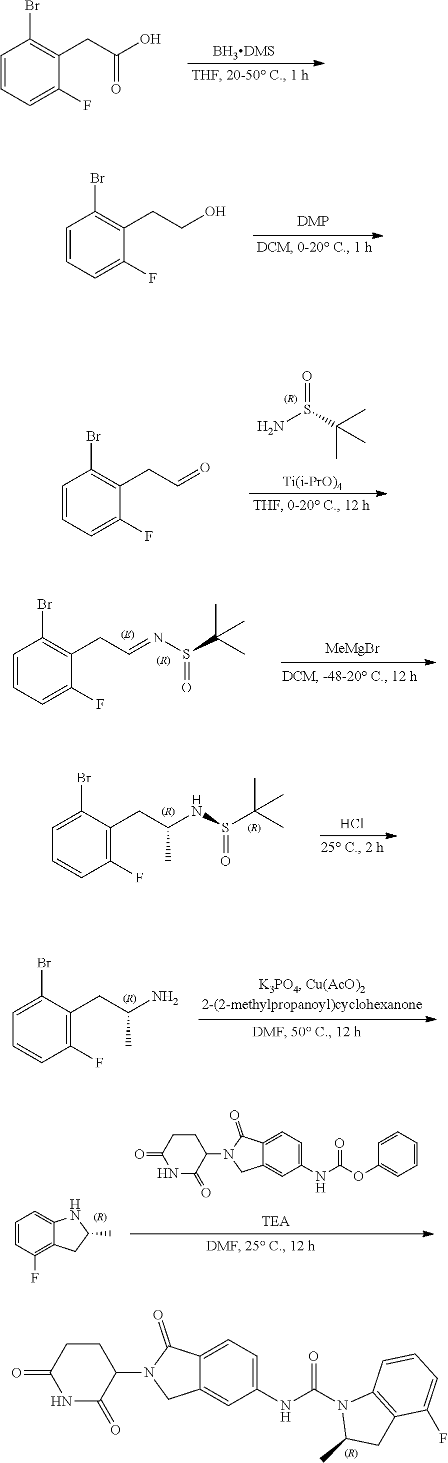

Step 1. To a solution of 2-(2-bromo-6-fluorophenyl)acetic acid (5.00 g, 21.4 mmol, 1.00 eq) in tetrahydrofuran (50.0 mL) was added borane dimethyl sulfide complex (10.0 M, 3.22 mL, 1.50 eq) under nitrogen at 20° C. The mixture was stirred at 50° C. for 1 h under nitrogen. The reaction mixture was quenched with methanol (10.0 mL) to give a solution. The solution was stirred at 80° C. for 0.5 h and concentrated under reduced pressure to give a residue. The residue was purified by column chromatography (SiO2, Petroleum ether/Ethyl acetate=8/1 to 0/1) to give 2-(2-bromo-6-fluorophenyl)ethanol (4.80 g, crude) as a yellow oil. ¹H NMR (400 MHz, DMSO-d₆) δ=7.47-7.40 (m, 1H), 7.25-7.16 (m, 2H), 4.86 (t, J=5.6 Hz, 1H), 3.56-3.50 (m, 2H), 2.91 (dt, J=2.0, 7.4 Hz, 2H).

Step 2. To a solution of 2-(2-bromo-6-fluorophenyl)ethanol (4.80 g, 21.90 mmol, 1.00 eq) in dichloromethane (20.0 mL) was Dess-Martin periodinane (13.9 g, 32.8 mmol, 10.1 mL, 1.50 eq) at 0° C. The mixture was stirred at 20° C. for 1 h. The reaction mixture was diluted with water (30 mL) and exacted with dichloromethane (2×30 mL). The organic phase was separated, washed with brine (10.0 mL), dried over sodium sulfate, filtered and concentrated under reduced pressure to give a residue. The residue was purified by column chromatography (SiO2, Petroleum ether/Ethyl acetate=8/1 to 0/1) to give 2-(2-bromo-6-fluorophenyl) acetaldehyde (3.9 g, 17.9 mmol, 82% yield) as yellow oil. ¹H NMR (400 MHz, DMSO-d₆) δ=9.73 (s, 1H), 7.51 (br d, J=7.8 Hz, 1H), 7.30 (br dd, J=8.1, 13.6 Hz, 2H), 4.02 (s, 2H).

Step 3. To a solution of 2-(2-bromo-6-fluorophenyl)acetaldehyde (3.38 g, 15.5 mmol, 1.00 eq) in tetrahydrofuran (30.0 mL) was added (R)-2-methylpropane-2-sulfinamide (2.08 g, 17.1 mmol, 1.10 eq), titanium(IV) isopropoxide (6.64 g, 23.3 mmol, 6.89 mL, 1.50 eq) at 0° C. The mixture was stirred at 20° C. for 12 h under nitrogen. The reaction mixture was diluted with water (30.0 mL) and exacted with ethyl acetate (2×30 mL). The organic phase was separated, washed with brine (10.0 mL), dried over sodium sulfate, filtered and concentrated under reduced pressure to give a residue. The residue was purified by column chromatography (SiO2, Petroleum ether/Ethyl acetate=8/1 to 5/1) to give (R,E)-N-(2-(2-bromo-6-fluorophenyl)ethylidene)-2-methylpropane-2-sulfinamide (2.80 g, 8.74 mmol, 56% yield) as a white oil. ¹H NMR (400 MHz, DMSO-d₆) δ=8.05 (t, J=3.3 Hz, 1H), 7.53-7.49 (m, 1H), 7.33-7.26 (m, 2H), 4.05 (td, J=2.5, 8.5 Hz, 2H), 1.01 (s, 9H).

Step 4. To a solution of (R,E)-N-(2-(2-bromo-6-fluorophenyl)ethylidene)-2-methylpropane-2-sulfinamide (1.50 g, 4.68 mmol, 1.00 eq) in dichloromethane (30 mL) was added methylmagnesium bromide (3.00 M, 7.81 mL, 5.00 eq) under nitrogen at −48° C. The mixture was stirred at 20° C. for 12 h. The mixture was quenched by addition saturated ammonium chloride (100 mL) at 0° C., then extracted with ethyl acetate (2×50 mL). The combined organic layers were washed with brine (20.0 mL), dried over sodium sulfate, filtered and concentrated under reduced pressure to give a residue. The residue was purified by reversed-phase HPLC (column: spherical C18, 20-45 um, 100 Å, SW 120, mobile phase: [water (0.1% Formic Acid)-ACN]) and lyophilized to give (R)—N—((R)-1-(2-bromo-6-fluorophenyl) propan-2-yl)-2-methylpropane-2-sulfinamide (1.70 g, 5.06 mmol, 53.96% yield) as a yellow solid. ¹H NMR (400 MHz, DMSO-d₆) δ=7.46-7.41 (m, 1H), 7.22-7.20 (m, 1H), 7.20-7.16 (m, 1H), 5.04 (d, J=9.0 Hz, 1H), 3.51-3.45 (m, 1H), 3.03-2.93 (m, 1H), 2.76 (ddd, J=1.9, 6.1, 13.4 Hz, 1H), 1.26 (d, J=6.5 Hz, 3H), 0.96 (s, 9H)

Step 5. A solution of (R)—N—((R)-1-(2-bromo-6-fluorophenyl)propan-2-yl)-2-methylpropane-2-sulfinamide (1.70 g, 5.06 mmol, 1.00 eq) in hydrochloric acid/dioxane (4 M, 10.0 mL) was stirred at 25° C. for 2 h. The mixture was filtered. The filtrate was purified by reversed-phase HPLC (column: spherical C18, 20-45 um, 100 Å, SW 120, mobile phase: [water (0.1% Formic Acid)-ACN]) and lyophilized to give (R)-1-(2-bromo-6-fluorophenyl)propan-2-amine (1.20 g, crude) as a yellow oil. ¹H NMR (400 MHz, DMSO-d₆) δ=8.14 (br s, 2H), 7.56-7.48 (m, 1H), 7.35-7.25 (m, 2H), 3.43 (dt, J=5.6, 10.6 Hz, 1H), 3.16-3.06 (m, 1H), 3.04-2.96 (m, 1H), 1.13 (s, 3H)

Step 6. To a solution of (R)-1-(2-bromo-6-fluorophenyl)propan-2-amine (200 mg, 861 umol, 1.00 eq) in dimethyl formamide (1.00 mL) was added 2-(2-methylpropanoyl)cyclohexanone (28.9 mg, 172 umol, 0.200 eq), potassium phosphate (365 mg, 1.72 mmol, 2.00 eq) and cupric acetate (7.83 mg, 43.0 umol, 0.05 eq) under nitrogen. The mixture was stirred at 50° C. for 12 h. The (R)-4-fluoro-2-methylindoline (130 mg, crude) in dimethyl formamide (1.00 mL) was obtained as black solution.

Step 7. To a solution of (R)-4-fluoro-2-methylindoline (130 mg, 859 umol, 1.00 eq) in dimethyl formamide (1.00 mL) was added phenyl (2-(2,6-dioxopiperidin-3-yl)-1-oxoisoindolin-5-yl)carbamate (326 mg, 859 umol, 1.00 eq) and triethylamine (261. mg, 2.58 mmol, 359 μL, 3.00 eq). The mixture was stirred at 25° C. for 12 h. The mixture was filtered. The filter liquor was purified by reversed-phase HPLC (column: spherical C18, 20-45 um, 100 Å, SW 80, mobile phase: [water (0.1% Formic Acid)-ACN]) and lyophilized to give (2R)—N-(2-(2,6-dioxopiperidin-3-yl)-1-oxoisoindolin-5-yl)-4-fluoro-2-methylindoline-1-carboxamide (49 mg, 112 umol, 13% yield) as a brown solid. ¹H NMR (400 MHz, DMSO-d₆) δ=10.98 (s, 1H), 9.03 (s, 1H), 7.89 (s, 1H), 7.70 (d, J=8.0 Hz, 1H), 7.65 (s, 2H), 7.24-7.17 (m, 1H), 6.79 (t, J=8.6 Hz, 1H), 5.09 (dd, J=4.9, 13.3 Hz, 1H), 5.03-4.94 (m, 1H), 4.49-4.38 (m, 1H), 4.36-4.25 (m, 1H), 3.42 (br d, J=9.1 Hz, 1H), 2.96-2.87 (m, 1H), 2.78 (br d, J=15.4 Hz, 1H), 2.60 (br dd, J=2.1, 15.4 Hz, 1H), 2.42-2.32 (m, 1H), 2.03-1.95 (m, 1H), 1.27 (d, J=6.1 Hz, 3H). MS (ESI) m/z 437.3 [M+H]⁺

### Example 58. Synthesis of Compound 350

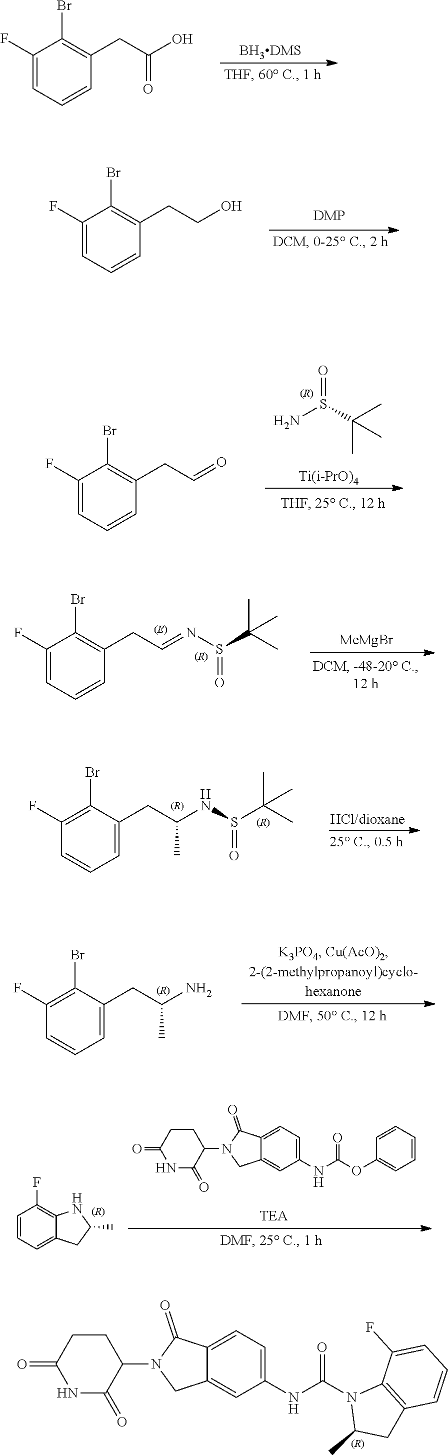

Step 1. To a solution of 2-(2-bromo-3-fluoro-phenyl)acetic acid (10.0 g, 42.9 mmol, 1.00 eq) in tetrahydrofuran (100 mL) was added borane dimethyl sulfide complex (10 M, 6.44 mL, 1.50 eq) at 0° C. under nitrogen atmosphere. Then the mixture was stirred at 60° C. for 1 h. The reaction mixture was added methanol (100 mL). Then the mixture was concentrated in vacuum. The residue was diluted with water (300 mL) and extracted with ethyl acetate (3×1500 mL). The combined organic layers were washed with brine (3×50 mL), dried over sodium sulfate, filtered and concentrated under reduced pressure to afford 2-(2-bromo-3-fluorophenyl)ethanol (9.50 g, crude) as yellow oil. ¹H NMR (400 MHz, DMSO-d₆) δ=7.40-7.30 (m, 1H), 7.26-7.15 (m, 2H), 4.78 (t, J=5.3 Hz, 1H), 3.62 (dt, J=5.4, 7.0 Hz, 2H), 2.91 (t, J=7.0 Hz, 2H).

Step 2. To a solution of 2-(2-bromo-3-fluoro-phenyl)ethanol (9.50 g, 43.3 mmol, 1.00 eq) in dichloromethane (200 mL) was added (1,1,1-triacetoxy)-1,1-dihydro-1,2-benziodoxol-3(1H)-one (36.7 g, 86.7 mmol, 26.8 mL, 2.00 eq) at 0° C. Then the mixture was stirred at 25° C. for 2 h. The reaction mixture was diluted with water (200 mL) and extracted with dichloromethane (3×100 mL). The combined organic layers were dried over sodium sulfate, filtered and concentrated under reduced pressure to give a residue. The residue was purified by column chromatography (SiO₂, Petroleum ether/Ethyl acetate=50/1 to 3/1) and concentrated in vacuum to afford 2-(2-bromo-3-fluorophenyl)acetaldehyde (9.60 g, 42.0 mmol, 96% yield, 95% purity) as yellow oil. ¹H NMR (400 MHz, DMSO-d₆) δ=9.78-9.66 (m, 1H), 7.43-7.38 (m, 1H), 7.35-7.31 (m, 1H), 7.23 (d, J=7.8 Hz, 1H), 4.04 (s, 2H).

Step 3. To a solution of 2-(2-bromo-3-fluorophenyl)acetaldehyde (9.60 g, 44.2 mmol, 1.00 eq) and (R)-2-methylpropane-2-sulfinamide (5.90 g, 48.6 mmol, 1.10 eq) in tetrahydrofuran (100 mL) was added tetraisopropoxytitanium (18.8 g, 66.3 mmol, 19.5 mL, 1.50 eq) at 0° C. Then the mixture was stirred at 25° C. for 12 h. The reaction mixture was diluted with water (200 mL) and extracted with dichloromethane (3×100 mL). The combined organic layers were dried over sodium sulfate, filtered and concentrated under reduced pressure to give a residue. The residue was purified by column chromatography (SiO₂, Petroleum ether/Ethyl acetate=50/1 to 5/1) and concentrated in vacuum to afford (R,E)-N-(2-(2-bromo-3-fluorophenyl)ethylidene)-2-methylpropane-2-sulfinamide (3.20 g, 9.99 mmol, 22% yield, 70% purity) as yellow oil. ¹H NMR (400 MHz, DMSO-d₆) δ=8.03 (t, J=3.9 Hz, 1H), 7.45-7.40 (m, 1H), 7.29-7.27 (m, 1H), 7.13-7.09 (m, 1H), 4.09 (d, J=3.9 Hz, 2H), 1.05 (s, 9H).

Step 4. To a solution of (R,E)-N-(2-(2-bromo-3-fluorophenyl)ethylidene)-2-methylpropane-2-sulfinamide (3.20 g, 9.99 mmol, 1.00 eq) in dichloromethan (50.0 mL) was added methylmagnesium bromide (3 M, 16.6 mL, 5.00 eq) at −48° C. under nitrogen atmosphere. Then the mixture was stirred at 25° C. for 12 h. The mixture was quenched by addition saturated ammonium chloride (300 mL) at 0° C., and then extracted with ethyl acetate (3×150 mL). The combined organic layers were washed with brine (3×50 mL), dried over sodium sulfate, filtered and concentrated under reduced pressure to give a residue. The residue was purified by reversed phase column chromatography (C18, 330 g; condition: water/acetonitrile=1/0 to 0/1, 0.1% formic acid) and lyophilized to afford (R)—N—((R)-1-(2-bromo-3-fluorophenyl)propan-2-yl)-2-methylpropane-2-sulfinamide (1.15 g, 3.39 mmol, 33% yield, 99% purity) as a white solid. ¹H NMR (400 MHz, DMSO-d₆) δ=7.33 (dt, J=5.8, 7.8 Hz, 1H), 7.24-7.14 (m, 2H), 5.02 (d, J=9.0 Hz, 1H), 3.56-3.44 (m, 1H), 2.98-2.88 (m, 1H), 2.87-2.81 (m, 1H), 1.28 (d, J=6.5 Hz, 3H), 0.94 (s, 9H).

Step 5. To a solution of (R)—N—((R)-1-(2-bromo-3-fluorophenyl)propan-2-yl)-2-methylpropane-2-sulfinamide (1.15 g, 3.42 mmol, 1.00 eq) in dioxane (10.0 mL) was added hydrochloride/dioxane (4 M, 12.7 mL, 14.9 eq). Then the reaction mixture was stirred at 25° C. for 0.5 h. The reaction mixture was concentrated in vacuum. The residue was diluted with sodium bicarbonate (100 mL) and extracted with ethyl acetate (3×50 mL). The combined organic layers were washed with brine (20 mL), dried over sodium sulfate, filtered and concentrated under reduced pressure to give a residue to afford (R)-1-(2-bromo-3-fluorophenyl)propan-2-amine (780 mg, 3.33 mmol, 97% yield, 99% purity) as yellow oil. MS (ESI) m/z. 234.2 [M+H]⁺

Step 6. To a solution of (R)-1-(2-bromo-3-fluorophenyl)propan-2-amine (50.0 mg, 215 umol, 1.00 eq) in dimethylformamide (0.500 mL) was added 2-(2-methylpropanoyl)cyclohexanone (7.25 mg, 43.0 umol, 0.200 eq), copper(II) acetate (1.96 mg, 10.7 umol, 0.0500 eq) and potassium phosphate (91.4 mg, 430 umol, 2.00 eq). Then the mixture was stirred at 50° C. for 12 h under nitrogen atmosphere. The crude product (R)-7-fluoro-2-methylindoline (230 mg, crude) in N,N-dimethylformamide (0.5 mL) was obtained as yellow solution.

Step 7. To a solution of phenyl (2-(2,6-dioxopiperidin-3-yl)-1-oxoisoindolin-5-yl)carbamate (88.3 mg, 232 umol, 1.10 eq) and (R)-7-fluoro-2-methylindoline (32.0 mg, 211 umol, 1.00 eq) in N,N-dimethylformamide (1.00 mL) was added triethylamine (64.2 mg, 635 umol, 88.3 uL, 3.00 eq). Then the mixture was stirred at 25° C. for 1 h. The reaction mixture was adjust pH to 5-6 by formic acid (0.2 mL) and filtered. The filtrate was purified by prep-HPLC (column: Phenomenex luna C18 150*25 mm*10 um; mobile phase: [water (FA)-ACN]; B %: 26%-56%, 9 min) and further purified by reversed phase column chromatography (C18, 40 g; condition: water/acetonitrile=1/0 to 0/1) and lyophilized to afford (2R)—N-(2-(2,6-dioxopiperidin-3-yl)-1-oxoisoindolin-5-yl)-7-fluoro-2-methylindoline-1-carboxamide (23.4 mg, 53.0 umol, 4% yield, 99% purity) as yellow solid. ¹H NMR (400 MHz, DMSO-d₆) δ=11.01-10.91 (m, 1H), 9.23 (s, 1H), 7.89-7.82 (m, 1H), 7.69-7.55 (m, 2H), 7.16-7.10 (m, 1H), 7.08-7.01 (m, 2H), 5.14-4.90 (m, 1H), 4.71-4.59 (m, 1H), 4.47-4.37 (m, 1H), 4.33-4.19 (m, 1H), 3.49-3.39 (m, 1H), 2.96-2.85 (m, 1H), 2.64-2.57 (m, 2H), 2.04-1.94 (m, 1H), 1.32-1.24 (m, 3H). MS (ESI) m/z 437.4 [M+H]⁺

### Example 59. Synthesis of Compound 358

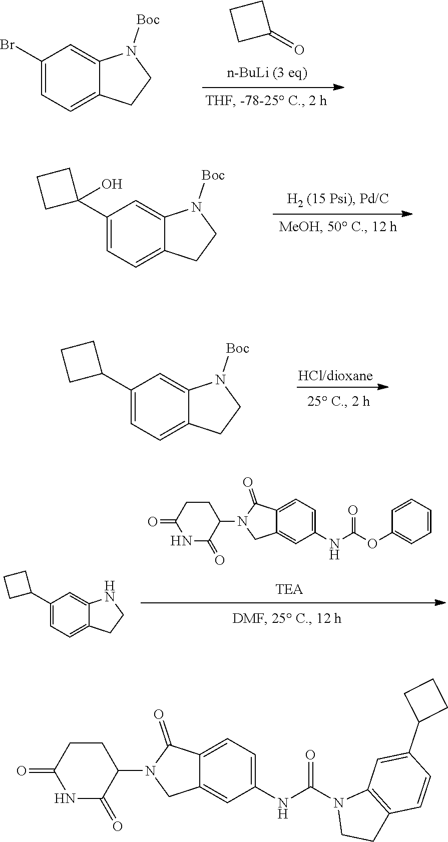

Step 1. To a solution of tert-butyl 6-bromoindoline-1-carboxylate (1.50 g, 5.03 mmol, 1.00 eq), 4A molecular sieve (15.0 g, 5.03 mmol, 1.00 eq) in tetrahydrofuran (20.0 mL) was added n-butyllithium (2.50 M, 6.04 mL, 3.00 eq) dropwise at −78° C. under nitrogen. The mixture was stirred at −78° C. for 0.5 h, then the mixture was added cyclobutanone (705 mg, 10.1 mmol, 752 μL, 2.00 eq) dropwise at −78° C. under nitrogen. The mixture was stirred at 25° C. for 2 h under nitrogen. The reaction mixture was quenched by addition saturated ammonium chloride aqueous solution (30 mL), and extracted with ethyl acetate (3×30 mL), the combined organic phase was washed with brine (30 mL), dried with anhydrous sodium sulfate, filtered and concentrated in vacuum. The residue was further purified by reversed-phase HPLC (0.1% formic acid condition) and lyophilized to give tert-butyl 6-(1-hydroxycyclobutyl)indoline-1-carboxylate (560 mg, 1.94 mmol, 38% yield) as a white solid.

¹H NMR (400 MHz, DMSO-d₆) δ=7.97-7.51 (m, 1H), 7.14-7.09 (m, 1H), 7.03 (dd, J=1.6, 7.7 Hz, 1H), 5.38 (s, 1H), 3.90 (t, J=8.7 Hz, 2H), 3.02 (br t, J=8.6 Hz, 2H), 2.37-2.30 (m, 2H), 2.28-2.20 (m, 2H), 1.97-1.85 (m, 1H), 1.63-1.58 (m, 1H), 1.51 (br s, 9H).

Step 2. To a solution of tert-butyl 6-(1-hydroxycyclobutyl)indoline-1-carboxylate (50.0 mg, 173 umol, 1.00 eq) in methanol (20.0 mL) was added palladium on carbon (200 mg, 10% purity) under nitrogen. The suspension was degassed under vacuum and purged with hydrogen several times. The mixture was stirred under hydrogen (15 Psi) at 50° C. for 12 h. The mixture was concentrated under reduced pressure to give tert-butyl 6-cyclobutylindoline-1-carboxylate (200 mg, 732 umol, 85% yield) as brown oil. ¹H NMR (400 MHz, DMSO-d₆) δ=7.68-7.29 (m, 1H), 7.08 (d, J=7.5 Hz, 1H), 6.75 (br d, J=7.4 Hz, 1H), 3.88 (t, J=8.6 Hz, 2H), 3.51-3.42 (m, 1H), 2.99 (br t, J=8.6 Hz, 2H), 2.32-2.21 (m, 2H), 2.04-1.97 (m, 2H), 1.87-1.74 (m, 2H), 1.51 (br s, 9H).

Step 3. A solution of tert-butyl 6-cyclobutylindoline-1-carboxylate (200 mg, 732 umol, 1.00 eq) in hydrochloric acid (4 M in dioxane) (10.0 mL) was stirred at 25° C. for 2 h. The mixture was concentrated under reduced pressure to give 6-cyclobutylindoline (200 mg, crude) as brown oil. ¹H NMR (400 MHz, DMSO-d₆) δ=7.37 (d, J=7.6 Hz, 1H), 7.27-7.21 (m, 2H), 3.69 (t, J=7.8 Hz, 2H), 3.59 (br s, 1H), 3.16-3.13 (m, 2H), 2.35-2.28 (m, 2H), 2.06-2.00 (m, 2H), 1.62 (br dd, J=4.6, 10.3 Hz, 2H).

Step 4. To a solution of 6-cyclobutylindoline (180 mg, 1.04 mmol, 1.30 eq), phenyl (2-(2,6-dioxopiperidin-3-yl)-1-oxoisoindolin-5-yl)carbamate (303 mg, 799 umol, 1.00 eq) in dimethyformamide (5.00 mL) was added triethylamine (323 mg, 3.20 mmol, 445 μL, 4.00 eq). The mixture was stirred at 25° C. for 12 h. The mixture was added formic acid (0.2 mL) to adjusted pH=3-4. The mixture was purified by prep-HPLC (Phenomenex luna C18 150×25 mm×10 um, mobile phase: 0.1% formic acid condition-acetonitrile, B %: 46%-66%, 58 min) and further purified by prep-HPLC (Welch Xtimate C18 150×25 mm×5 um, mobile phase: 0.1% hydrochloric acid condition-acetonitrile, B %: 34%-54%, 13 min), then lyophilized to give a solid. The solid was purified by prep-NPLC (Welch Ultimate XB-Diol 250×50×10 um, mobile phase: Hexane-ethanol, B %: 17%-50%, 20 min) and concentrated under reduced pressure. The residue was purified by reverse phase chromatography (column: spherical C18, 20-45 um, 100 Å, SW 40, mobile phase: [water (0.1% Formic Acid)-ACN) and lyophilized to give 6-cyclobutyl-N-(2-(2,6-dioxopiperidin-3-yl)-1-oxoisoindolin-5-yl)indoline-1-carboxamide (28.5 mg, 62.2 umol, 8% yield) as a white solid. ¹H NMR (400 MHz, DMSO-d₆) δ=11.02-10.93 (m, 1H), 8.82 (s, 1H), 7.94 (s, 1H), 7.82 (s, 1H), 7.64 (s, 2H), 7.11 (d, J=7.5 Hz, 1H), 6.77 (dd, J=1.1, 7.6 Hz, 1H), 5.09 (dd, J=5.1, 13.3 Hz, 1H), 4.47-4.40 (m, 1H), 4.33-4.27 (m, 1H), 4.16 (t, J=8.6 Hz, 2H), 3.54-3.44 (m, 2H), 3.13 (br t, J=8.5 Hz, 2H), 2.97-2.86 (m, 1H), 2.68-2.58 (m, 2H), 2.39 (br dd, J=4.4, 13.2 Hz, 1H), 2.31-2.23 (m, 2H), 2.10-2.02 (m, 2H), 1.85-1.74 (m, 1H). MS (ESI) m/z. 459.1 [M+H]⁺

### Example 60. Synthesis of Compound 347

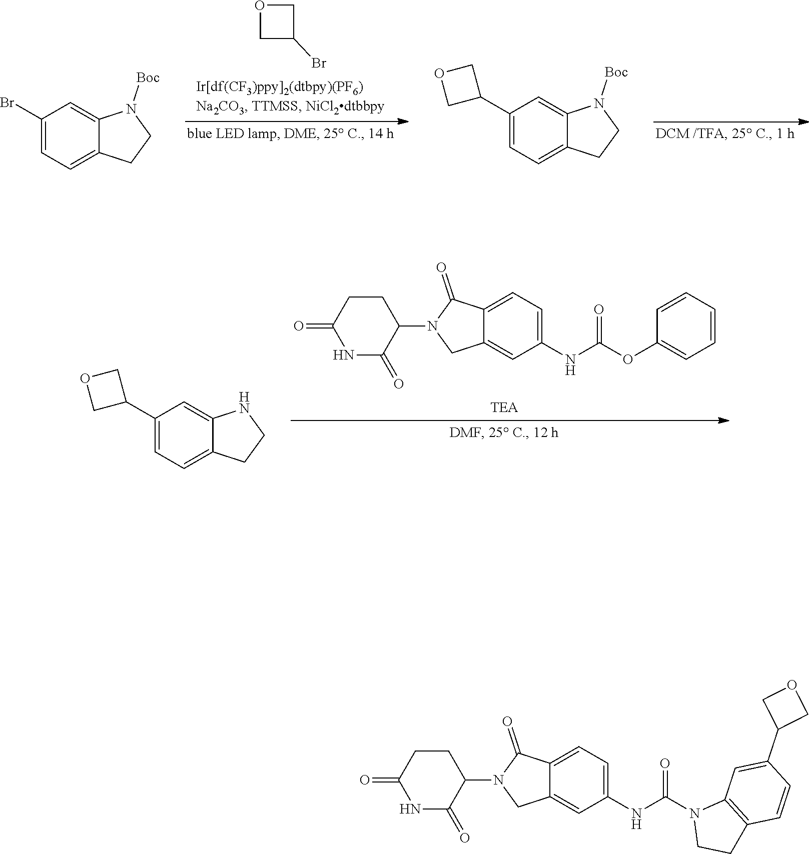 [SMILES: BrC1COC1 ; CC(C)(C)OC(=O)N1CCc2ccc(Br)cc21 ; CC(C)(C)OC(=O)N1CCc2ccc(C3COC3)cc21 ; c1cc2c(cc1C1COC1)NCC2 ; O=C1CCC(N2Cc3cc(NC(=O)Oc4ccccc4)ccc3C2=O)C(=O)N1 ; O=C1CCC(N2Cc3cc(NC(=O)N4CCc5ccc(C6COC6)cc54)ccc3C2=O)C(=O)N1]

Step 1. To an 15.0 mL vial equipped with a stir bar was added tert-butyl 6-bromoindoline-1-carboxylate (2.00 g, 6.71 mmol, 1.00 eq), 3-bromooxetane (1.19 g, 8.72 mmol, 1.30 eq), Ir[dF(CF₃)ppy]₂(dtbpy)(PF₆) (75.3 mg, 67.1 umol, 0.010 eq), nickel (II) chloride.dtbbpy (40.0 mg, 100 umol, 0.015 eq), tris(trimethylsilyl)silane (1.67 g, 6.71 mmol, 2.07 mL, 1.00 eq), sodium carbonate (1.42 g, 13.4 mmol, 2.00 eq) in 1,2-dimethoxyethane (20.0 mL). The vial was sealed and placed under nitrogen was added. The reaction was stirred and irradiated with a 10 W blue LED lamp (3.00 cm away), with cooling water to keep the reaction temperature at 25° C. for 14 h. The mixture was concentrated in vacuum. The reaction mixture was diluted with water (200 mL) and extracted with ethyl acetate (3×200 mL). The combined organic layers were washed with brine (200 mL), dried over sodium sulfate, filtered and concentrated under reduced pressure to give a residue. The crude product was purified by reversed-phase HPLC (column: spherical C18, 20-45 um, 100 Å, SW 120, mobile phase: [H2O and ACN) and lyophilized to give tert-butyl 6-(oxetan-3-yl)indoline-1-carboxylate (700 mg, 2.54 mmol, 37% yield) as yellow oil. ¹H NMR (400 MHz, DMSO-d₆) δ=7.85-7.62 (m, 1H), 7.15 (d, J=7.5 Hz, 1H), 6.90 (br d, J=7.3 Hz, 1H), 4.93 (dd, J=5.8, 8.3 Hz, 2H), 4.55 (dd, J=6.0, 6.6 Hz, 2H), 4.19 (quin, J=7.5 Hz, 1H), 3.90 (t, J=8.7 Hz, 2H), 3.02 (t, J=8.6 Hz, 2H), 1.51 (br s, 9H).

Step 2. To a mixture of tert-butyl 6-(oxetan-3-yl)indoline-1-carboxylate (300 mg, 1.09 mmol, 1.00 eq) in dichloromethane (5.00 mL) was added triethylamine (1.54 g, 13.5 mmol, 1.00 mL, 12.4 eq). The mixture was stirred at 25° C. for 1 h. The mixture was concentrated in vacuum to give 6-(oxetan-3-yl)indoline (190 mg, crude) as yellow oil. MS (ESI) m/z. 176.0 [M+H]⁺

Step 3. To a mixture of 6-(oxetan-3-yl)indoline (121 mg, 690 umol, 1.00 eq) and phenyl (2-(2,6-dioxopiperidin-3-yl)-1-oxoisoindolin-5-yl)carbamate (157 mg, 414 umol, 0.600 eq) in dimethyformamide (1.00 mL) was added triethylamine (209 mg, 2.07 mmol, 288 μL, 3.00 eq). The mixture was stirred at 25° C. for 12 h. The mixture was filtered. The filtrate was purified by reversed-phase HPLC (column: spherical C18, 20-45 um, 100 Å, SW 120, mobile phase: [water (0.1% Formic Acid)-ACN) and lyophilized. The residue was purified by prep-HPLC (column: Phenomenex luna C18 150*25 mm*10 um; mobile phase: [water (FA)-MeOH]; B %: 20%-50%, 10 min) and lyophilized to give N-(2-(2,6-dioxopiperidin-3-yl)-1-oxoisoindolin-5-yl)-6-(oxetan-3-yl)indoline-1-carboxamide (10.63 mg, 22.1 umol, 3% yield, 96% purity) as an off-white solid. ¹H NMR (400 MHz, DMSO-d₆) δ=10.98 (br s, 1H), 8.86 (s, 1H), 8.07-7.85 (m, 2H), 7.65 (s, 2H), 7.19 (d, J=7.5 Hz, 1H), 6.93 (dd, J=1.3, 7.6 Hz, 1H), 5.09 (dd, J=5.1, 13.2 Hz, 1H), 4.94 (dd, J=5.9, 8.4 Hz, 2H), 4.58 (t, J=6.3 Hz, 2H), 4.47-4.39 (m, 1H), 4.33-4.26 (m, 1H), 4.26-4.14 (m, 3H), 3.17 (br t, J=8.3 Hz, 2H), 2.97-2.86 (m, 1H), 2.63-2.58 (m, 1H), 2.42-2.33 (m, 1H), 2.06-1.95 (m, 1H). MS (ESI) m/z. 461.3 [M+H]⁺

### Example 61. Synthesis of Compound 364

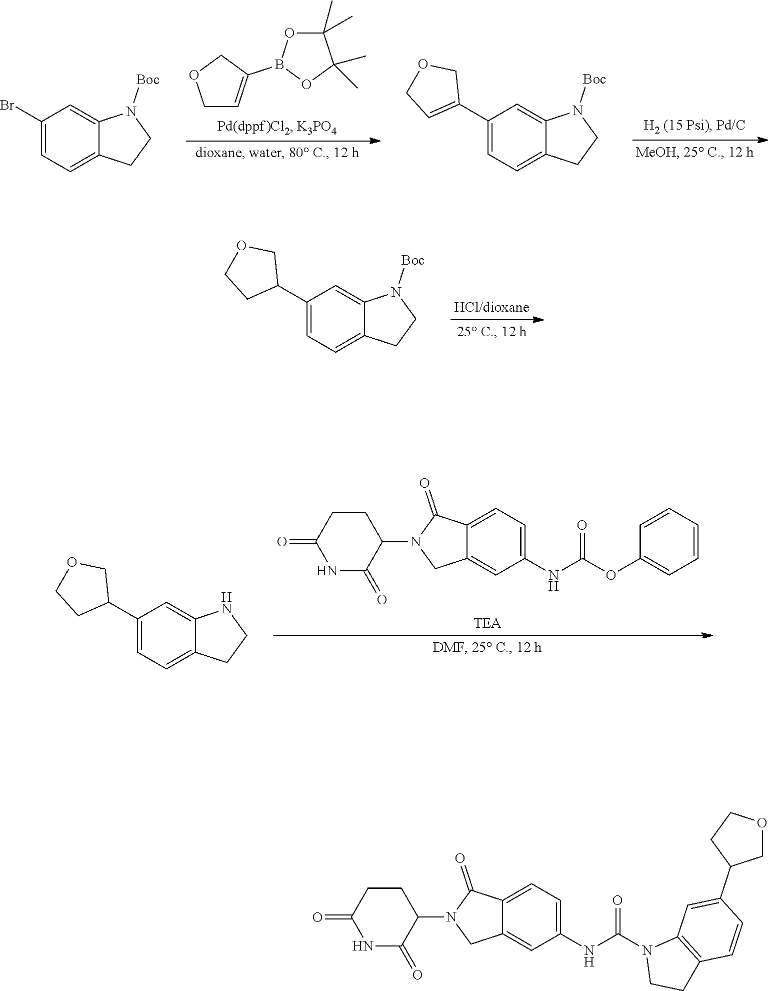

Step 1. To a solution of tert-butyl 6-bromoindoline-1-carboxylate (500 mg, 1.68 mmol, 1.00 eq) in dioxane (10.0 mL) and water (1.00 mL) was added 2-(2,5-dihydrofuran-3-yl)-4,4,5,5-tetramethyl-1,3,2-dioxaborolane (493 mg, 2.52 mmol, 1.50 eq), potassium phosphate (1.07 g, 5.03 mmol, 3.00 eq) and [1,1-bis(diphenylphosphino) ferrocene]dichloropalladium(II) (122 mg, 167 umol, 0.100 eq) under nitrogen atmosphere. The mixture was stirred at 80° C. for 12 h. The reaction mixture was diluted with water (30 mL) and exacted with ethyl acetate (2×30 mL). The organic phase was separated, washed with brine (10 mL), dried over sodium sulfate, filtered and concentrated under reduced pressure to give a residue which was purified by reversed-phase HPLC (column: spherical C18, 20-45 um, 100 Å, SW 80, mobile phase: [water (0.1% Formic Acid)-ACN]) and lyophilized to give tert-butyl 6-(2,5-dihydrofuran-3-yl)indoline-1-carboxylate (400 mg, 1.39 mmol, 83% yield) as a yellow solid. ¹H NMR (400 MHz, DMSO-d₆) δ=7.83-7.44 (m, 1H), 7.18 (d, J=7.8 Hz, 1H), 7.00 (br d, J=7.3 Hz, 1H), 6.35 (br s, 1H), 4.90-4.83 (m, 2H), 4.72 (br d, J=3.6 Hz, 2H), 3.91 (t, J=8.6 Hz, 2H), 3.04 (br t, J=8.6 Hz, 2H), 1.52 (br s, 9H).

Step 2. To a solution of tert-butyl 6-(2,5-dihydrofuran-3-yl)indoline-1-carboxylate (402 mg, 1.40 mmol, 1.00 eq) in methanol (50.0 mL) was added palladium on carbon (50.0 mg, 10% purity) under hydrogen (15 Psi). The mixture was stirred at 25° C. for 12 h. The mixture was filtered. The filtrate was concentrated under reduced pressure to give tert-butyl 6-(tetrahydrofuran-3-yl)indoline-1-carboxylate (437 mg, crude) as a black solid.

¹H NMR (400 MHz, DMSO-d₆) δ=7.70-7.50 (m, 1H), 7.10 (d, J=7.6 Hz, 1H), 6.82 (d, J=7.6 Hz, 1H), 4.00 (t, J=7.8 Hz, 1H), 3.94-3.85 (m, 3H), 3.77 (q, J=7.8 Hz, 1H), 3.51 (t, J=7.8 Hz, 1H), 3.33-3.28 (m, 1H), 3.00 (br t, J=8.6 Hz, 2H), 2.33-2.24 (m, 1H), 1.85 (dd, J=8.1, 12.2 Hz, 1H), 1.50 (br s, 9H).

Step 3. A solution of tert-butyl 6-(tetrahydrofuran-3-yl)indoline-1-carboxylate (436 mg, 1.51 mmol, 1.00 eq) in hydrochloric acid (4 M in dioxane) (5.00 mL) was stirred at 25° C. for 2 h. The reaction mixture was concentrated under reduced pressure to give 6-(tetrahydrofuran-3-yl)indoline (273 mg, 1.44 mmol, 95% yield) as a black solid. ¹H NMR (400 MHz, DMSO-d₆) δ=11.89-11.37 (m, 1H), 7.42-7.36 (m, 1H), 7.34-7.29 (m, 2H), 4.02 (t, J=7.6 Hz, 1H), 3.95 (dt, J=4.6, 8.3 Hz, 1H), 3.79 (q, J=7.7 Hz, 1H), 3.68 (t, J=7.8 Hz, 2H), 3.56-3.51 (m, 1H), 3.50-3.41 (m, 1H), 3.15 (t, J=7.7 Hz, 2H), 2.33 (dtd, J=4.6, 7.7, 12.2 Hz, 1H), 1.87 (qd, J=7.9, 12.2 Hz, 1H).

Step 4. To a solution of phenyl (2-(2,6-dioxopiperidin-3-yl)-1-oxoisoindolin-5-yl)carbamate (133 mg, 352 umol, 1.00 eq) in dimethyl formamide (1.00 mL) was added 6-(tetrahydrofuran-3-yl)indoline (100 mg, 528 umol, 1.50 eq) and triethylamine (107 mg, 1.06 mmol, 147 μL, 3.00 eq). The mixture was stirred at 25° C. for 12 h. The mixture was filtered. The filter liquor was purified by prep-HPLC (column: Phenomenex luna C18 150*25 mm*10 um; mobile phase: [water (FA)-ACN]; B %: 26%-56%, 9 min) and lyophilized to give N-(2-(2,6-dioxopiperidin-3-yl)-1-oxoisoindolin-5-yl)-6-(tetrahydrofuran-3-yl)indoline-1-carboxamide (33.1 mg, 67.05 umol, 19% yield, 96% purity) as an off-white solid. ¹H NMR (400 MHz, DMSO-d₆) δ=10.98 (s, 1H), 8.84 (s, 1H), 7.97-7.81 (m, 2H), 7.64 (s, 2H), 7.13 (d, J=7.5 Hz, 1H), 6.84 (d, J=7.6 Hz, 1H), 5.09 (dd, J=5.1, 13.2 Hz, 1H), 4.47-4.40 (m, 1H), 4.35-4.26 (m, 1H), 4.16 (br t, J=8.5 Hz, 2H), 4.01 (t, J=7.9 Hz, 1H), 3.93 (dt, J=4.6, 8.2 Hz, 1H), 3.78 (q, J=7.7 Hz, 1H), 3.51 (br t, J=7.8 Hz, 2H), 3.14 (br t, J=8.4 Hz, 2H), 2.96-2.86 (m, 1H), 2.62 (br s, 1H), 2.39 (br dd, J=4.4, 13.3 Hz, 1H), 2.29 (tdd, J=3.8, 8.0, 12.0 Hz, 1H), 2.04-1.97 (m, 1H), 1.91-1.83 (m, 1H). MS (ESI) m/z 475.2 [M+H]⁺

### Example 62. Synthesis of Compound 365

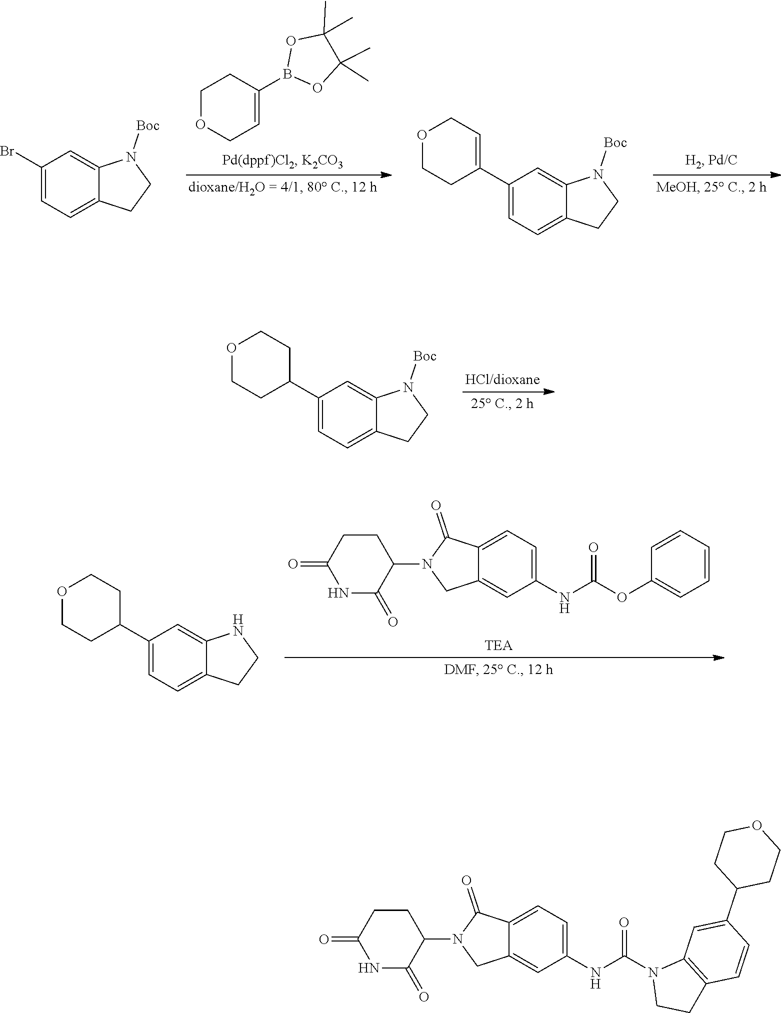

Step 1. To a mixture of tert-butyl 6-bromoindoline-1-carboxylate (500 mg, 1.68 mmol, 1.00 eq) and 2-(3,6-dihydro-2H-pyran-4-yl)-4,4,5,5-tetramethyl-1,3,2-dioxaborolane (423 mg, 2.01 mmol, 1.20 eq) in dioxane (5.00 mL) and water (1.00 mL) was added potassium phosphate (1.07 g, 5.03 mmol, 3.00 eq) and [1,1-bis(diphenylphosphino)ferrocene]dichloropalladium(II) (123 mg, 168 umol, 0.100 eq). The mixture was stirred at 80° C. for 12 h under nitrogen. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by column chromatography (SiO₂, Petroleum ether/Ethyl acetate=20/0 to 1/1) and concentrated in vacuum to give tert-butyl 6-(3,6-dihydro-2H-pyran-4-yl)indoline-1-carboxylate (400 mg, 1.33 mmol, 79% yield) as yellow oil. ¹H NMR (400 MHz, DMSO-d₆) δ=7.95-7.64 (m, 1H), 7.15 (d, J=7.8 Hz, 1H), 7.01 (dd, J=1.3, 7.8 Hz, 1H), 6.15 (br s, 1H), 4.21 (q, J=2.6 Hz, 2H), 3.90 (t, J=8.6 Hz, 2H), 3.81 (t, J=5.4 Hz, 2H), 3.03 (br t, J=8.6 Hz, 2H), 2.40 (br dd, J=2.6, 4.3 Hz, 2H), 1.51 (br s, 9H).

Step 2. To a solution of tert-butyl 6-(3,6-dihydro-2H-pyran-4-yl)indoline-1-carboxylate (380 mg, 1.26 mmol, 1.00 eq) in methanol (6.00 mL) was added palladium on carbon (400 mg, 1.26 mmol, 10% purity, 1.00 eq) under nitrogen atmosphere. The suspension was degassed and purged with hydrogen for 3 times. The mixture was stirred under hydrogen (15 Psi) at 25° C. for 2 h. The mixture was filterted. The filtrate was concentrated in vacuum to give tert-butyl 6-(tetrahydro-2H-pyran-4-yl)indoline-1-carboxylate (300 mg, 988 umol, 78% yield) as yellow solid. MS (ESI) m/z. 248.0 [M−55]⁺

Step 3. A mixture of tert-butyl 6-(tetrahydro-2H-pyran-4-yl)indoline-1-carboxylate (300 mg, 989 umol, 1.00 eq) in hydrochloric acid (4 M in dioxane) (5.00 mL) was stirred at 25° C. for 2 h. The mixture was concentrated in vacuum to give 6-(tetrahydro-2H-pyran-4-yl)indoline (240 mg, crude) as yellow oil. ¹H NMR (400 MHz, DMSO-d₆) δ=7.40 (d, J=8.3 Hz, 1H), 7.31-7.28 (m, 2H), 3.95 (br dd, J=2.8, 10.2 Hz, 2H), 3.71-3.68 (m, 2H), 3.46 (br s, 2H), 3.17-3.13 (m, 2H), 2.85 (br dd, J=4.9, 10.6 Hz, 1H), 1.69-1.66 (m, 2H), 1.64 (br d, J=4.1 Hz, 1H), 1.21-1.16 (m, 2H).

Step 4. To a mixture of 6-(tetrahydro-2H-pyran-4-yl)indoline (100 mg, 492 umol, 1.20 eq) and phenyl (2-(2,6-dioxopiperidin-3-yl)-1-oxoisoindolin-5-yl)carbamate (156 mg, 409 umol, 1.00 eq) in dimethyformamide (1.00 mL) was added triethylamine (124 mg, 1.23 mmol, 171 μL, 3.00 eq). The mixture was stirred at 25° C. for 12 h. The mixture was quenched by formic acid (0.5 mL) and filtered. The filtrate was purified by reversed-phase HPLC (0.1% formic acid condition) and lyophilized to give N-(2-(2,6-dioxopiperidin-3-yl)-1-oxoisoindolin-5-yl)-6-(tetrahydro-2H-pyran-4-yl)indoline-1-carboxamide (53.7 mg, 109 umol, 26% yield) as a white solid. ¹H NMR (400 MHz, DMSO-d₆) δ=10.98 (br s, 1H), 8.83 (s, 1H), 8.44 (s, 0.1H), 7.95 (s, 1H), 7.84 (d, J=1.0 Hz, 1H), 7.64 (s, 2H), 7.13 (d, J=7.6 Hz, 1H), 6.82 (dd, J=1.3, 7.6 Hz, 1H), 5.09 (dd, J=5.1, 13.3 Hz, 1H), 4.48-4.39 (m, 1H), 4.34-4.26 (m, 1H), 4.16 (t, J=8.6 Hz, 2H), 3.94 (br dd, J=2.9, 10.5 Hz, 2H), 3.42 (dt, J=2.9, 11.3 Hz, 2H), 3.14 (br t, J=8.4 Hz, 2H), 2.97-2.86 (m, 1H), 2.75-2.67 (m, 1H), 2.60 (br dd, J=2.0, 15.5 Hz, 1H), 2.42-2.32 (m, 1H), 2.06-1.94 (m, 1H), 1.69-1.57 (m, 4H). MS (ESI) m/z. 489.2 [M+H]⁺

### Example 63. Synthesis of Compound 338

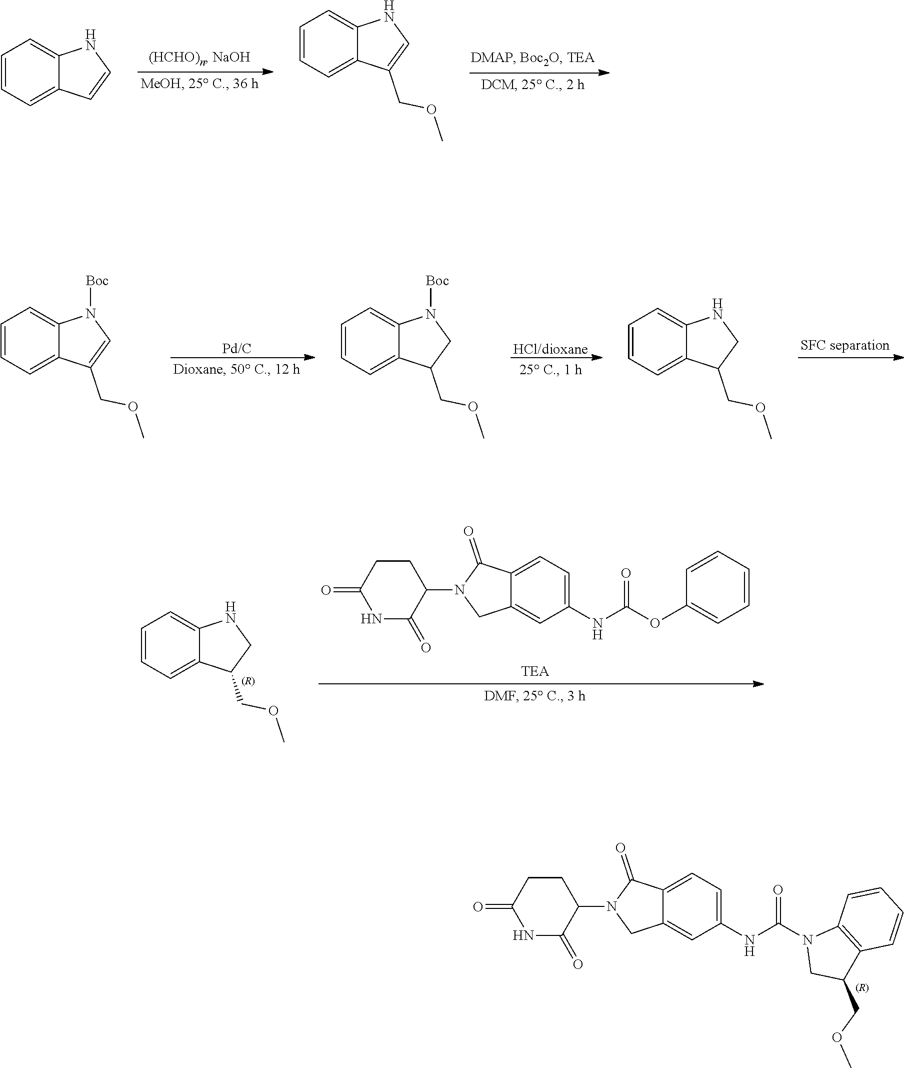

Step 1. To a solution of 1H-indole (5.00 g, 42.6 mmol, 1.00 eq) in methanol (50.0 mL) was added paraformaldehyde (2.56 g, 85.3 mmol, 2.00 eq) and sodium hydroxide (3.41 g, 85.3 mmol, 2.00 eq). The mixture was stirred at 25° C. for 24 h. Then paraformaldehyde (1.28 g, 42.6 mmol, 1.00 eq) and sodium hydroxide (1.71 g, 42.6 mmol, 1.00 eq) was added into the mixture and the mixture was stirred at 25° C. for 12 h. The mixture was concentrated under reduced pressure to give a residue and the residue purified by column chromatography on silica gel eluted with petroleum ether/ethyl acetate=20/1 to 1/1. The desired fraction was collected and concentrated reduced pressure to afford 3-(methoxymethyl)-1H-indole (2.00 g, 12.4 mmol, 5% yield) as yellow oil. ¹H NMR (400 MHz, DMSO-d₆) δ=11.00 (br s, 1H), 7.56 (d, J=7.5 Hz, 1H), 7.36 (d, J=8.1 Hz, 1H), 7.33 (d, J=2.4 Hz, 1H), 7.09 (dt, J=1.1, 7.6 Hz, 1H), 7.02-6.97 (m, 1H), 4.55 (s, 2H), 3.23 (s, 3H)

Step 2. To a solution of 3-(methoxymethyl)-1H-indole (2.60 g, 16.1 mmol, 1.00 eq) in dichloromethane (20.0 mL) was added di-tert-butyl dicarbonate (3.52 g, 16.1 mmol, 3.71 mL, 1.00 eq), 4-dimethylaminopyridin (197 mg, 1.61 mmol, 0.100 eq) and triethylamine (4.90 g, 48.3 mmol, 6.73 mL, 3.00 eq). The mixture was stirred at 25° C. for 2 h. The mixture was concentrated under reduced pressure to give a residue. The residue was purified by column chromatography on silica gel eluted with petroleum ether/ethyl acetate=20/1 to 1/1. The desired fraction was collected and concentrated reduced pressure to afford tert-butyl 3-(methoxymethyl)-1H-indole-1-carboxylate (3.00 g, 11.4 mmol, 71% yield) was obtained as yellow oil. ¹H NMR (400 MHz, DMSO-d₆) δ=8.05 (d, J=8.3 Hz, 1H), 7.67-7.60 (m, 2H), 7.38-7.30 (m, 1H), 7.29-7.21 (m, 1H), 4.56 (s, 2H), 3.28 (s, 3H), 1.63 (s, 9H).

Step 3. To a solution of tert-butyl 3-(methoxymethyl)-1H-indole-1-carboxylate (1.00 g, 3.83 mmol, 1.00 eq) in dioxane (5.00 mL) was added palladium on activated carbon (10%)(wetted with ca. 55% Water) (100 mg, 10% purity). The mixture was stirred at 50° C. for 12 h. The mixture was filtered to give the filtrate, and the filtrate was concentrated under reduced pressure to give a residue. The residue was purified by column chromatography on silica gel eluted with petroleum ether/ethyl acetate=1/0 to 10/1. The desired fraction was collected and concentrated reduced pressure to afford tert-butyl 3-(methoxymethyl)indoline-1-carboxylate (2.60 g, 9.87 mmol, 86% yield) as pink oil. ¹H NMR (400 MHz, DMSO-d₆) δ=7.81-7.42 (m, 1H), 7.24 (d, J=7.3 Hz, 1H), 7.17 (t, J=7.7 Hz, 1H), 6.93 (t, J=7.4 Hz, 1H), 4.01-3.93 (m, 1H), 3.71-3.65 (m, 1H), 3.60-3.50 (m, 2H), 3.43-3.39 (m, 1H), 3.27 (s, 3H), 1.50 (s, 9H)

Step 4. To a solution of tert-butyl 3-(methoxymethyl)indoline-1-carboxylate (2.60 g, 9.87 mmol, 1.00 eq) in hydrochloride/dioxane (4.00 M, 20.0 mL, 8.10 eq). The mixture was stirred at 25° C. for 1 h. The mixture was filtered to give the filtrate and the filtrate was purified by reversed-phase HPLC (Cis, 80 g; condition: water/acetonitrile=1/0 to 0/1, 0.1% formic acid). The desired fraction was collected and lyophilized to afford 3-(methoxymethyl)indoline (1.60 g, 9.12 mmol, 92% yield, 93% purity) as a yellow solid. ¹H NMR (400 MHz, DMSO-d₆) δ=11.59-10.34 (m, 1H), 7.45 (br d, J=4.8 Hz, 1H), 7.34 (br d, J=5.8 Hz, 3H), 3.81-3.74 (m, 1H), 3.74-3.66 (m, 1H), 3.62-3.58 (m, 1H), 3.52 (br d, J=7.5 Hz, 1H), 3.48 (br s, 1H), 3.28 (s, 3H). MS (ESI) m/z 164.2 [M+H]⁺

Step 5. The 3-(methoxymethyl)indoline (1.60 g, 9.12 mmol, 93% purity) was purified by Superitical fluid chromatogram (column: DAICEL CHIRALPAK IA (250 mm*30 mm, 10 um); mobile phase: [IPA (0.1% IPAm)]; B %: 1%-1%, 12 min). The desired fraction was collected and concentrated reduced pressure to afford (R)-3-(methoxymethyl)indoline as yellow oil. MS (ESI) m/z 164.2 [M+H]⁺

Step 6. To a solution of (R)-3-(methoxymethyl)indoline (100 mg, 612 umol, 1.00 eq) and phenyl (2-(2,6-dioxopiperidin-3-yl)-1-oxoisoindolin-5-yl)carbamate (232 mg, 612 umol, 1.00 eq) in N,N-dimethyl formamide (2.00 mL) was added triethylamine (185 mg, 1.84 mmol, 255 uL, 3.00 eq). The mixture was stirred at 25° C. for 3 h. Then the mixture was adjusted pH=5 with formic acid (0.500 mL). The crude product was purified by Prep-HPLC (column: Phenomenex luna C18 150*25 mm*10 um; mobile phase: [water (formic acid)-acetonitrile]; B %: 25%-55%, 10 min) and lyophilized to afford (3R)—N-(2-(2,6-dioxopiperidin-3-yl)-1-oxoisoindolin-5-yl)-3-(methoxymethyl)indoline-1-carboxamide (22.3 mg, 47.7 umol, 7% yield, 96% purity) as a white solid. ¹H NMR (400 MHz, DMSO-d₆) δ=10.97 (s, 1H), 8.93-8.83 (m, 1H), 7.99-7.86 (m, 2H), 7.74-7.60 (m, 2H), 7.27 (d, J=7.4 Hz, 1H), 7.18 (t, J=7.6 Hz, 1H), 6.94 (t, J=7.4 Hz, 1H), 5.09 (dd, J=5.2, 13.3 Hz, 1H), 4.48-4.39 (m, 1H), 4.33-4.22 (m, 2H), 4.02 (dd, J=5.7, 10.2 Hz, 1H), 3.76-3.67 (m, 1H), 3.63 (dd, J=4.9, 9.1 Hz, 1H), 3.45 (t, J=8.7 Hz, 1H), 3.32 (br s, 3H), 2.97-2.85 (m, 1H), 2.65-2.56 (m, 1H), 2.41-2.36 (m, 1H), 2.05-1.95 (m, 1H). MS (ESI) m/z 449.0 [M+H]⁺

### Example 64. Synthesis of Compound 288

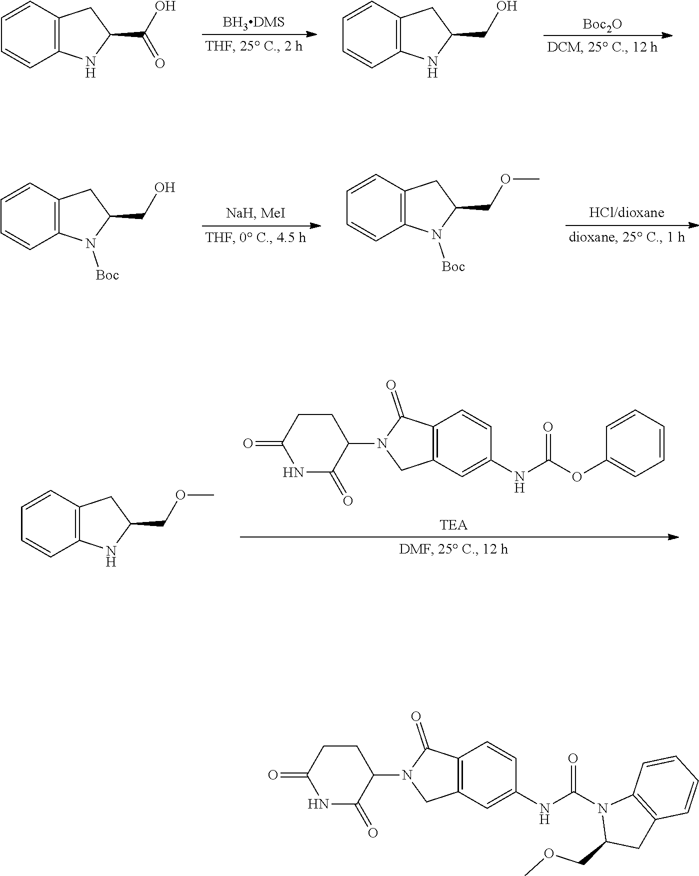

Step 1. To a solution of (S)-indoline-2-carboxylic acid (5.00 g, 30.6 mmol, 1.00 eq) in tetrahydrofuran (50 mL) was added borane dimethyl sulfide complex (10.0 M, 6.13 mL, 2.00 eq) at 0° C. under nitrogen atmosphere. The mixture was stirred at 25° C. for 2 h. After being cooled to room temperature, the mixture was added methyl alcohol (10 mL), then the mixture was stirred at 50° C. for 0.5 h. The mixture was concentrated under reduced pressure to give a residue. The crude product was purified by column chromatography on silica gel eluted with petroleum ether/ethyl acetate=50/1 to 0/1 to afford (S)-indolin-2-ylmethanol (2.40 g, 16.1 mmol, 52% yield) as a black solid. ¹H NMR (400 MHz, DMSO-d₆) δ=6.96 (br d, J=7.1 Hz, 1H), 6.87 (br t, J=7.5 Hz, 1H), 6.52-6.42 (m, 2H), 5.50 (br s, 1H), 4.74 (t, J=5.2 Hz, 1H), 3.81-3.71 (m, 1H), 3.35-3.33 (m, 1H), 2.96 (br dd, J=9.2, 15.8 Hz, 1H), 2.63 (br dd, J=6.7, 15.8 Hz, 1H)

Step 2. To a solution of (S)-indolin-2-ylmethanol (2.40 g, 16.1 mmol, 1.00 eq) in dichloromethane (10.0 mL) was added di-tert-butyl dicarbonate (3.86 g, 17.7 mmol, 4.07 mL, 1.10 eq). The mixture was stirred at 25° C. for 12 h. After being cooled to room temperature, the mixture was diluted with water (200 mL) and extracted with ethyl acetate (3×100 mL). The organic layers were collected and was washed with brine (50.0 mL), dried over anhydrous sodium sulfate and evaporated to dryness. The crude product was purified by column chromatography on silica gel eluted with petroleum ether/ethyl acetate=50/1 to 0/1 to afford (S)-tert-butyl 2-(methoxymethyl)indoline-1-carboxylate (4.00 g, 16.0 mmol, 99% yield) as yellow oil. ¹H NMR (400 MHz, DMSO-d₆) δ=7.77-7.41 (m, 1H), 7.18-7.08 (m, 2H), 6.90 (t, J=7.3 Hz, 1H), 4.87 (br t, J=5.6 Hz, 1H), 4.37-4.30 (m, 1H), 3.60-3.52 (m, 1H), 3.38-3.34 (m, 1H), 3.20 (br dd, J=9.9, 16.3 Hz, 1H), 2.99 (br d, J=16.4 Hz, 1H), 1.50 (s, 9H).

Step 3. To a solution of (S)-tert-butyl 2-(hydroxymethyl)indoline-1-carboxylate (4.00 g, 16.0 mmol, 1.00 eq) in tetrahydrofuran (5.00 mL) was added sodium hydride (706 mg, 17.7 mmol, 60% purity, 1.10 eq) at 0° C. under nitrogen atmosphere. The mixture was stirred at 0° C. for 0.5 h. Then methyl iodide (9.12 g, 64.3 mmol, 4.00 mL, 4.00 eq) was added into the mixture, the mixture was stirred at 0° C. for 4 h. The mixture was quenched with saturated ammonium chloride in aqueous solution (50 mL), then extracted with ethyl acetate (3×100 mL). The organic layers were collected and dried over anhydrous sodium sulfate and evaporated to dryness. The crude product was purified by column chromatography on silica gel eluted with petroleum ether/ethyl acetate=50/1 to 0/1 to afford (S)-tert-butyl 2-(methoxymethyl)indoline-1-carboxylate (1.30 g, 4.94 mmol, 30% yield) as yellow solid. ¹H NMR (400 MHz, DMSO-d₆) δ=7.56 (br s, 1H), 7.19-7.08 (m, 2H), 6.91 (t, J=7.4 Hz, 1H), 4.48 (tdd, J=3.0, 6.6, 9.9 Hz, 1H), 3.47 (dd, J=3.6, 9.5 Hz, 1H), 3.35 (br d, J=2.5 Hz, 1H), 3.28 (br s, 1H), 3.24 (s, 3H), 2.89 (dd, J=2.1, 16.4 Hz, 1H), 1.51 (s, 9H)

Step 4. A solution of (S)-tert-butyl 2-(methoxymethyl)indoline-1-carboxylate (1.30 g, 4.94 mmol, 1.00 eq) in hydrochloric acid/dioxane (3.00 mL) was stirred at 25° C. for 2 h. The mixture was concentrated under reduced pressure to afford (S)-2-(methoxymethyl)indoline (1.30 g, crude) as a yellow solid.

Step 5. To a solution of (S)-2-(methoxymethyl)indoline (600 mg, 3.68 mmol, 1.00 eq), phenyl (2-(2,6-dioxopiperidin-3-yl)-1-oxoisoindolin-5-yl)carbamate (1.39 g, 3.68 mmol, 1.00 eq) in N,N-dimethyl formamide (3.00 mL) was added triethylamine (1.12 g, 11.0 mmol, 1.54 mL, 3.00 eq). The mixture was stirred at 25° C. for 12 h. The mixture was filtered. The filtrate was purified by reversed-phase HPLC (column: spherical C18, 20-45 um, 100 Å, SW 120, mobile phase: [water (0.1% Formic Acid)-ACN]; B %: 15%-45%, 30 min) to afford (2S)—N-(2-(2,6-dioxopiperidin-3-yl)-1-oxoisoindolin-5-yl)-2-(methoxymethyl)indoline-1-carboxamide (499 mg, 1.10 mmol, 29% yield, 99% purity) as an off-white solid. ¹H NMR (400 MHz, DMSO-d₆) δ=10.98 (s, 1H), 9.16 (s, 1H), 8.22 (s, 0.1H), 7.82 (s, 1H), 7.76 (d, J=8.0 Hz, 1H), 7.65 (d, J=8.4 Hz, 1H), 7.53 (br d, J=8.5 Hz, 1H), 7.22 (d, J=7.3 Hz, 1H), 7.15 (t, J=7.7 Hz, 1H), 6.94 (t, J=7.3 Hz, 1H), 5.09 (dd, J=5.0, 13.3 Hz, 1H), 4.97-4.89 (m, 1H), 4.47-4.40 (m, 1H), 4.33-4.26 (m, 1H), 3.50-3.45 (m, 2H), 3.39 (br d, J=10.0 Hz, 1H), 3.37 (br s, 3H), 2.95-2.81 (m, 2H), 2.60 (br d, J=17.3 Hz, 1H), 2.39 (br dd, J=4.4, 13.3 Hz, 1H), 2.04-1.95 (m, 1H). MS (ESI) m/z 449.4 [M+H]⁺

### Example 65. Synthesis of Compound 353

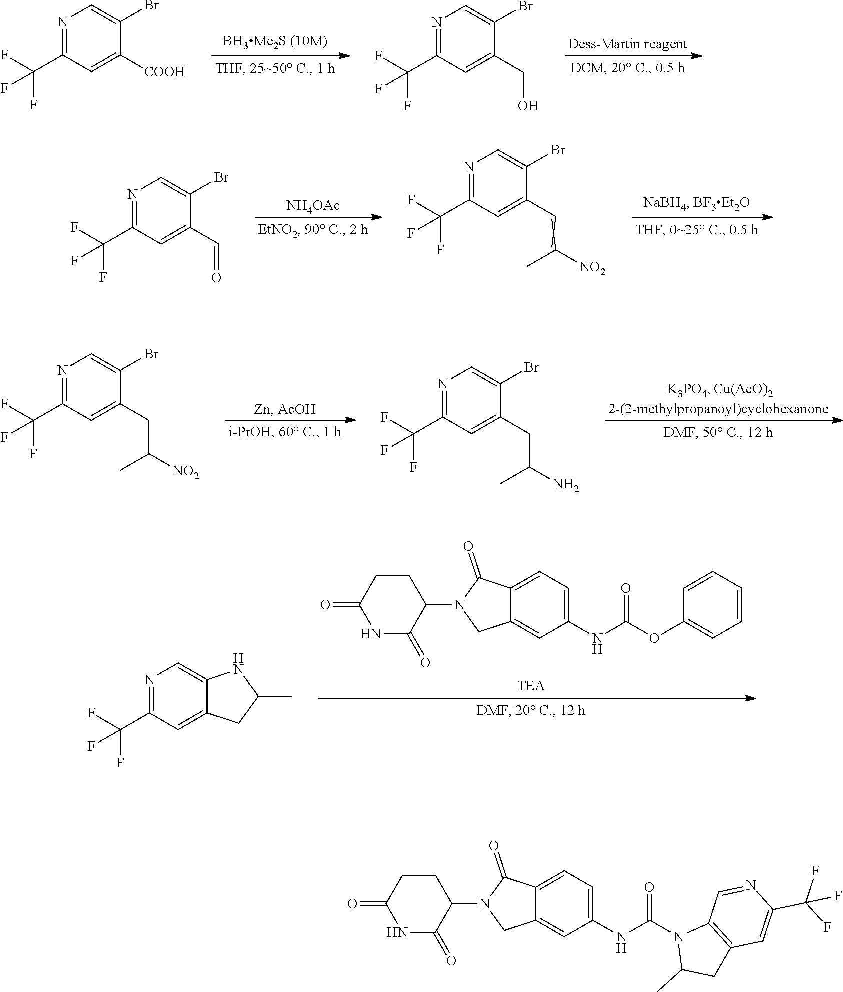

Step 1. To a solution of 5-bromo-2-(trifluoromethyl)isonicotinic acid (5.00 g, 18.5 mmol, 1.00 eq) in tetrahydrofuran (50 mL) was added borane dimethyl sulfide complex (10 M, 3.70 mL, 2.00 eq) at 20° C. The mixture was stirred at 50° C. for 1 h under nitrogen. The reaction mixture was quenched with methanol (15 mL) to give a solution. The solution was concentrated under reduced pressure to give a residue. The residue was dissolved with methanol (20 mL) and stirred at 80° C. for 0.5 h and concentrated under reduced pressure to give (5-bromo-2-(trifluoromethyl)pyridin-4-yl)methanol (4.80 g, crude) as yellow oil.

Step 2. To a solution of (5-bromo-2-(trifluoromethyl)pyridin-4-yl)methanol (4.80 g, 18.7 mmol, 1.00 eq) in dichloromethane (50 mL) was added Dess-Martin periodinane (11.9 g, 28.1 mmol, 8.71 mL, 1.50 eq). The mixture was stirred under nitrogen at 20° C. for 0.5 h. The mixture was filtered to give a filter liquor, then concentrated under reduced pressure to give a residue. The residue was purified by column chromatography (SiO₂, Petroleum ether/Ethyl acetate=1/0 to 8/1) to give 5-bromo-2-(trifluoromethyl)isonicotinaldehyde (4.50 g, 17.7 mmol, 94% yield) as yellow oil. ¹H NMR (400 MHz, DMSO-d₆) δ=10.18 (s, 1H), 9.19 (s, 1H), 8.10 (s, 1H).

Step 3. To a solution of 5-bromo-2-(trifluoromethyl)isonicotinaldehyde (500 mg, 1.97 mmol, 1.00 eq) in nitroethane (5.25 g, 69.9 mmol, 5.00 mL, 35.5 eq) was added ammonium acetate (300 mg, 3.89 mmol, 1.98 eq). The mixture was stirred at 90° C. for 2 h. After being cooled to room temperature, the mixture was diluted with ethyl acetate (100 mL) and water (50 mL). The organic layer was separated and concentrated under reduced pressure. The residue was purified by column chromatography on silica gel (petroleum ether/ethyl acetate=20/1) to give 5-bromo-4-(2-nitroprop-1-en-1-yl)-2-(trifluoromethyl)pyridine (280 mg, 900 umol, 45% yield) as colorless oil. ¹H NMR (400 MHz, CDCl₃) δ=8.94 (s, 1H), 7.97 (s, 1H), 7.58 (s, 1H), 2.34 (s, 3H). MS (ESI) m/z 313.0 [M+H]⁺

Step 4. To a solution of 5-bromo-4-(2-nitroprop-1-en-1-yl)-2-(trifluoromethyl)pyridine (150 mg, 482 umol, 1.00 eq) in tetrahydrofuran (20 mL) was added sodium borohydride (35.0 mg, 925 umol, 1.92 eq) and boron trifluoride diethyl etherate (138 mg, 972 umol, 120 μL, 2.02 eq). The mixture was stirred at 25° C. for 0.5 h under nitrogen. The reaction was quenched with methanol (5.00 mL) and concentrated under reduced pressure. The residue was purified by reverse phase HPLC (column: spherical C18, 20-45 um, 100 Å, SW 40, mobile phase: [water (0.1% Formic Acid)-ACN). The desired fraction was collected and lyophilized to give 5-bromo-4-(2-nitropropyl)-2-(trifluoromethyl)pyridine (50.0 mg, 159 umol, 33% yield) as yellow oil. ¹H NMR (400 MHz, CDCl3) δ=8.83 (s, 1H), 7.52 (s, 1H), 5.00-4.84 (m, 1H), 3.52 (dd, J=8.9, 14.3 Hz, 1H), 3.24 (dd, J=5.3, 14.2 Hz, 1H), 1.69 (d, J=6.6 Hz, 3H). MS (ESI) m/z 313.0 [M+H]⁺

Step 5. To a solution of 5-bromo-4-(2-nitropropyl)-2-(trifluoromethyl)pyridine (520 mg, 1.66 mmol, 1.00 eq) in isopropanol (20 mL) was added acetic acid (2.73 g, 45.4 mmol, 2.60 mL, 27.3 eq) and zinc powder (500 mg, 7.65 mmol, 4.60 eq). The mixture was stirred at 60° C. for 1 h. The mixture was filtered to give a solution which was concentrated under reduced pressure. The residue was purified by reverse phase HPLC (column: spherical C18, 20-45 um, 100 Å, SW 120, mobile phase: [water (0.1% Formic Acid)-ACN). The desired fraction was collected and dyophilized to give 1-(5-bromo-2-(trifluoromethyl)pyridin-4-yl) propan-2-amine (150 mg, 446 umol, 26% yield, 98% purity, formate) as a white solid. ¹H NMR (400 MHz, DMSO-d₆) δ=8.92 (s, 1H), 8.32 (s, 1H), 7.96 (s, 1H), 3.40-3.31 (m, 1H), 2.96 (d, J=6.5 Hz, 2H), 1.12 (d, J=6.4 Hz, 3H). MS (ESI) m/z 282.9 [M+H]⁺

Step 6. To a solution of 1-(5-bromo-2-(trifluoromethyl)pyridin-4-yl)propan-2-amine (100 mg, 353 umol, 1.00 eq) in dimethyl formamide (0.500 mL) was added 2-(2-methylpropanoyl)cyclohexanone (11.8 mg, 70.6 umol, 0.200 eq), potassium phosphate (149 mg, 706 umol, 2.00 eq) and copper(II)acetate (3.21 mg, 17.6 umol, 0.0500 eq). The mixture was stirred under nitrogen at 50° C. for 12 h. The mixture was filtered to give a filter liquor which was purified by reversed-phase HPLC (column: spherical C18, 20-45 um, 100 Å, SW 25, mobile phase: [water (0.1% Formic Acid)-ACN]) to give 2-methyl-5-(trifluoromethyl)-2,3-dihydro-1H-pyrrolo[2,3-c]pyridine (30.0 mg, 148 umol, 42% yield) as a white solid. ¹H NMR (400 MHz, DMSO-d₆) δ=7.78 (s, 1H), 7.45 (s, 1H), 6.50 (br s, 1H), 4.07-3.97 (m, 1H), 3.19 (dd, J=9.2, 17.1 Hz, 1H), 2.61 (dd, J=7.2, 16.9 Hz, 1H), 1.19 (d, J=6.1 Hz, 3H). MS (ESI) m/z 203.3 [M+H]⁺

Step 7. To a solution of 2-methyl-5-(trifluoromethyl)-2,3-dihydro-1H-pyrrolo[2,3-c]pyridine (30.0 mg, 148 umol, 1.00 eq) in dimethyl formamide (1.00 mL) was added phenyl (2-(2,6-dioxopiperidin-3-yl)-1-oxoisoindolin-5-yl)carbamate (61.9 mg, 163 umol, 1.10 eq) and triethylamine (45.0 mg, 445 umol, 61.9 uL, 3.00 eq). The mixture was stirred at 20° C. for 12 h. The mixture was filtered. The filter liquor was purified by Prep-HPLC (column: Phenomenex luna C18 150*25 mm*10 um; mobile phase: [water (FA)-ACN]; B %: 25%-55%, 10 min) and lyophilized to give N-(2-(2,6-dioxopiperidin-3-yl)-1-oxoisoindolin-5-yl)-2-methyl-5-(trifluoromethyl)-2,3-dihydro-1H-pyrrolo[2,3-c]pyridine-1-carboxamide (5 mg, 10.3 umol, 6% yield) as a yellow solid. ¹H NMR (400 MHz, DMSO-d₆) δ=11.08-10.89 (m, 1H), 9.24 (s, 1H), 9.13 (s, 1H), 7.93 (s, 1H), 7.83 (s, 1H), 7.69 (s, 2H), 5.13-4.99 (m, 2H), 4.50-4.40 (m, 1H), 4.37-4.27 (m, 1H), 3.56 (br dd, J=9.7, 17.6 Hz, 1H), 2.97-2.86 (m, 2H), 2.60 (br d, J=17.8 Hz, 1H), 2.42-2.36 (m, 1H), 2.06-1.96 (m, 1H), 1.28 (br d, J=6.1 Hz, 3H). MS (ESI) m/z 488.4 [M+H]⁺

### Example 66. Synthesis of Compound 356

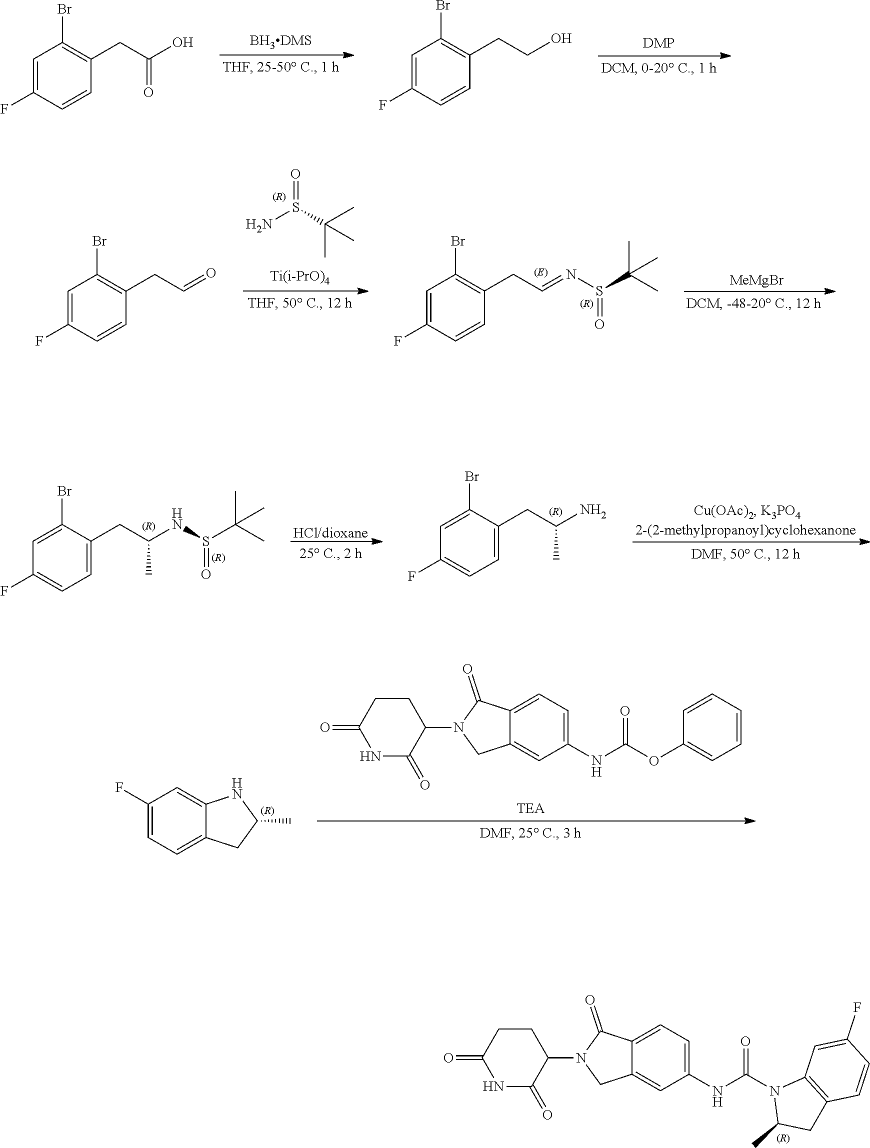

Step 1. To a solution of 2-(2-bromo-4-fluorophenyl)acetic acid (10.0 g, 42.9 mmol, 1.00 eq) in tetrahydrofuran (100 mL) was added borane dimethyl sulfide complex (10.0 M, 6.44 mL, 1.50 eq) dropwise at 25° C. The mixture was stirred at 50° C. for 1 h under nitrogen. The reaction mixture was quenched with methanol (10 mL) and concentrated under reduced pressure to give 2-(2-bromo-4-fluorophenyl)ethanol (9.00 g, crude) as brown oil. ¹H NMR (400 MHz, DMSO-d₆) δ=7.50 (dd, J=2.6, 8.6 Hz, 1H), 7.38 (br d, J=2.3 Hz, 1H), 7.19-7.15 (m, 1H), 3.60-3.56 (m, 3H), 2.81 (br s, 2H).

Step 2. To a solution of 2-(2-bromo-4-fluorophenyl)ethanol (8.00 g, 36.5 mmol, 1.00 eq) in dichloromethane (10.0 mL) was added 3,5-dimethyl-1H-pyrazole (23.2 g, 54.8 mmol, 17.0 mL, 1.50 eq) at 0° C. The mixture was stirred at 20° C. for 1 h under nitrogen. The reaction mixture was filtered. The filtrate was added saturated sodium sulfite solution (100 mL) and extracted with ethyl acetate (3×100 mL), the combined organic phase was washed with brine (80 mL), dried with anhydrous sodium sulfate, filtered and concentrated in vacuum. The residue was purified by column chromatography (SiO2, Petroleum ether/Ethyl acetate=1/0 to 20/1) and concentrated in vacuum to give 2-(2-bromo-4-fluorophenyl)acetaldehyde (4.00 g, 18.4 mmol, 50% yield) as yellow oil. ¹H NMR (400 MHz, DMSO-d₆) δ=9.69 (s, 1H), 7.61 (dd, J=2.6, 8.6 Hz, 1H), 7.43 (dd, J=6.3, 8.5 Hz, 1H), 7.31-7.23 (m, 1H), 3.95 (s, 2H).

Step 3. A solution of 2-(2-bromo-4-fluorophenyl)acetaldehyde (4.20 g, 19.4 mmol, 1.00 eq) in tetrahydrofuran (100 mL) was added titanium(iv) isopropoxide (8.25 g, 29.0 mmol, 8.57 mL, 1.50 eq) and (R)-2-methylpropane-2-sulfinamide (2.58 g, 21.3 mmol, 1.10 eq) at 0° C. The mixture was stirred at 50° C. for 12 h under nitrogen. The reaction mixture was diluted with water (50 mL) and extracted with ethyl acetate (3×50 mL). The organic phase was separated, washed with brine (50 mL), dried over sodium sulfate, filtered and concentrated under reduced pressure to give a residue. The residue was purified by silica gel chromatography (Petroleum ether/Ethyl acetate=1/0 to 10/1) and concentrated in vacuum to give (R,E)-N-(2-(2-bromo-4-fluorophenyl)ethylidene)-2-methylpropane-2-sulfinamide (2.60 g, 8.12 mmol, 41% yield) as yellow oil. ¹H NMR (400 MHz, DMSO-d₆) δ=8.00 (t, J=3.9 Hz, 1H), 7.60 (dd, J=2.8, 8.6 Hz, 1H), 7.48 (dd, J=6.2, 8.6 Hz, 1H), 7.27 (dt, J=2.8, 8.5 Hz, 1H), 4.01 (d, J=3.9 Hz, 2H), 1.05 (s, 9H).

Step 4. To a solution of (R,E)-N-(2-(2-bromo-4-fluorophenyl)ethylidene)-2-methylpropane-2-sulfinamide (2.10 g, 6.56 mmol, 1.00 eq) in dichloromethane (30 mL) was added methylmagnesium bromide (3.00 M, 10.9 mL, 5 eq) dropwise at −48° C. under nitrogen. The mixture was stirred at 20° C. for 12 h. The mixture was quenched by addition saturated ammonium chloride (30 mL) at 0° C., then extracted with ethyl acetate (30 mL×3). The combined organic layers were washed with brine (20 mL), dried over sodium sulfate, filtered and concentrated under reduced pressure to give a residue. The residue was purified by reversed-phase HPLC (column: spherical C18, 20-45 um, 100 Å, SW 120, mobile phase:[water (0.1% Formic Acid)-ACN]) and lyophilized to give (R)—N—((R)-1-(2-bromo-4-fluorophenyl)propan-2-yl)-2-methylpropane-2-sulfinamide (1.50 g, 4.46 mmol, 68% yield) as yellow oil. ¹H NMR (400 MHz, DMSO-d₆) δ=7.50 (dd, J=2.5, 8.6 Hz, 1H), 7.36 (dd, J=6.4, 8.5 Hz, 1H), 7.18 (dt, J=2.5, 8.5 Hz, 1H), 4.98 (d, J=9.1 Hz, 1H), 3.47-3.44 (m, 1H), 2.87-2.80 (m, 1H), 2.80-2.73 (m, 1H), 1.26 (d, J=6.5 Hz, 3H), 0.94 (s, 9H).

Step 5. A solution of (R)—N—((R)-1-(2-bromo-4-fluorophenyl)propan-2-yl)-2-methylpropane-2-sulfinamide (1.50 g, 4.46 mmol, 1.00 eq) in hydrochloric acid (4.00 M in dioxane, 15.0 mL). The mixture was stirred at 25° C. for 2 h. The mixture was filtered and the filtrate was concentrated under reduced pressure to give a residue. The residue was purified by reversed-phase HPLC (column: spherical C18, 20-45 um, 100 Å, SW 120, mobile phase: [water (0.1% Formic Acid)-ACN)) and lyophilized to give (R)-1-(2-bromo-4-fluorophenyl)propan-2-amine (450 mg, 1.94 mmol, 43% yield) as a white solid. ¹H NMR (400 MHz, DMSO-d₆) δ=7.60 (dd, J=2.6, 8.6 Hz, 1H), 7.49-7.40 (m, 1H), 7.27 (dt, J=2.7, 8.5 Hz, 1H), 3.43 (br d, J=6.4 Hz, 1H), 3.39 (br d, J=6.4 Hz, 2H), 3.04 (br dd, J=5.8, 13.6 Hz, 1H), 2.86 (dd, J=8.6, 13.6 Hz, 1H), 1.12 (d, J=6.5 Hz, 3H). MS (ESI) m/z. 233.9 [M+H]⁺

Step 6. A solution of (R)-1-(2-bromo-4-fluorophenyl)propan-2-amine (100 mg, 431 umol, 1.00 eq) and potassium phosphate (183 mg, 862 umol, 2.00 eq) in dimethyformamide (2.00 mL) was added 2-(2-methylpropanoyl)cyclohexanone (14.5 mg, 86.2 umol, 0.200 eq) and cupric acetate (3.91 mg, 21.5 umol, 0.0500 eq) under nitrogen. The mixture was stirred at 50° C. for 12 h under nitrogen. The mixture was used into the next step without further purification.

Step 7. To a solution of (R)-6-fluoro-2-methylindoline (150 mg, 992 umol, 1.00 eq) in in dimethyformamide (2.00 mL), phenyl (2-(2,6-dioxopiperidin-3-yl)-1-oxoisoindolin-5-yl)carbamate (376 mg, 992 umol, 1.00 eq) in dimethyformamide (2.00 mL) was added triethylamine (402 mg, 3.97 mmol, 552 μL, 4.00 eq). The mixture was stirred at 25° C. for 3 h. The mixture was added formic acid (0.5 mL) to adjusted pH=3-4. The mixture was purified by reverse phase chromatography (column: spherical C18, 20-45 um, 100 Å, SW 40, mobile phase: [water (0.1% Formic Acid)-ACN) and lyophilized to give (2R)—N-(2-(2,6-dioxopiperidin-3-yl)-1-oxoisoindolin-5-yl)-6-fluoro-2-methylindoline-1-carboxamide (22.3 mg, 50.7 umol, 5% yield, 99% purity) as a white solid. ¹H NMR (400 MHz, DMSO-d₆) δ=11.07-10.85 (m, 1H), 9.02 (s, 1H), 7.90 (s, 1H), 7.68-7.60 (m, 3H), 7.26-7.19 (m, 1H), 6.80-6.70 (m, 1H), 5.13-5.02 (m, 1H), 5.01-4.91 (m, 1H), 4.49-4.38 (m, 1H), 4.35-4.25 (m, 1H), 3.41-3.34 (m, 2H), 2.97-2.85 (m, 1H), 2.70 (br d, J=16.1 Hz, 1H), 2.34-2.28 (m, 1H), 2.05-1.95 (m, 1H), 1.25 (br d, J=6.1 Hz, 3H). MS (ESI) m/z. 437.4 [M+H]⁺

### Example 67. Synthesis of Compound 300

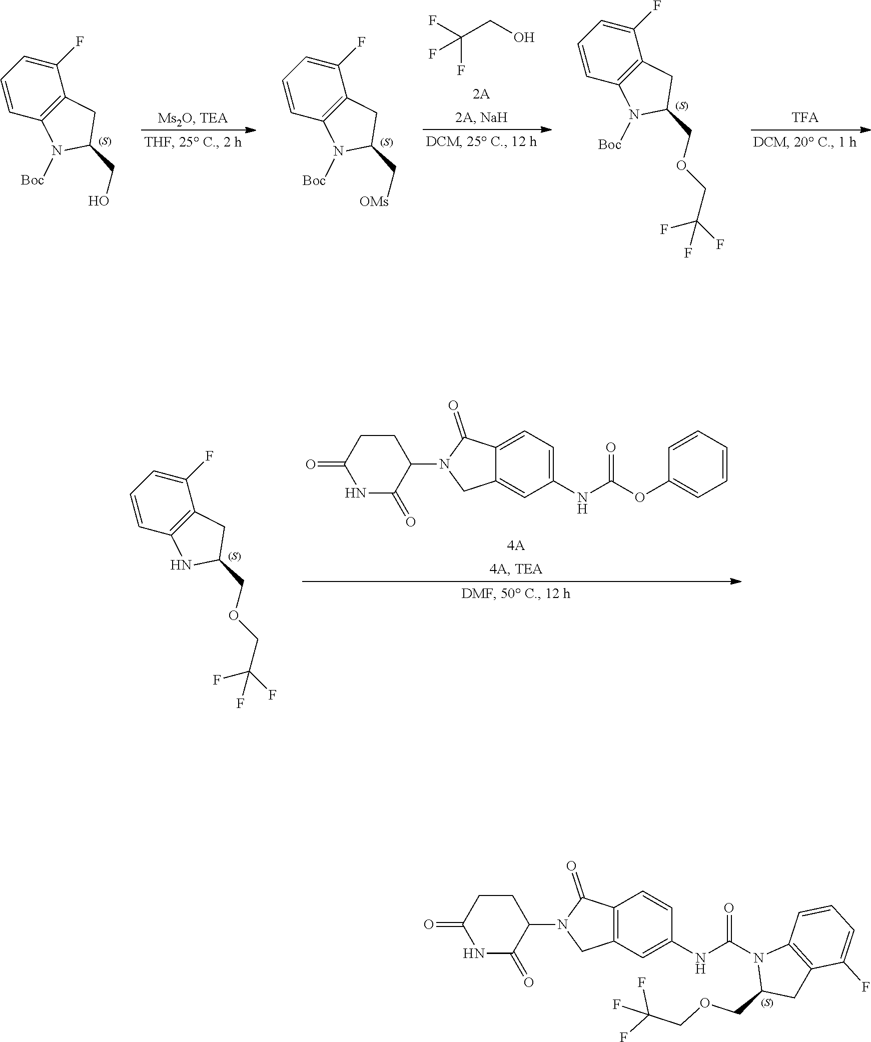

Step 1. To a solution of tert-butyl (S)-4-fluoro-2-(hydroxymethyl)indoline-1-carboxylate (110 mg, 412 mol, 1.00 eq) in tetrahydrofuran (1.00 mL) was added triethylamine (125 mg, 1.23 mmol, 172 μL, 3.00 eq) and methylsulfonyl methanesulfonate (108 mg, 617 mol, 1.50 eq), the mixture was stirred at 25° C. for 2 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to afford tert-butyl (S)-4-fluoro-2-((methylsulfonyl)methyl)indoline-1-carboxylate (100 mg, 290 mol, 70% yield) as yellow oil. ¹H NMR (400 MHz, DMSO-d₆) δ=7.48-7.33 (m, 1H), 7.24-7.16 (m, 1H), 6.80 (t, J=8.8 Hz, 1H), 4.79-4.68 (m, 1H), 4.44-4.37 (m, 1H), 4.34-4.30 (m, 1H), 3.39 (dd, J=10.4, 16.8 Hz, 1H), 3.14 (s, 3H), 2.96 (dd, J=3.2, 16.8 Hz, 1H), 1.52 (s, 9H). MS (ESI) m/z 246.0 [M−56+H]⁺

Step 2. To a solution of 2,2,2-trifluoroethan-1-ol (579 mg, 5.79 mmol, 416 μL, 20.0 eq) in tetrahydrofuran (1.00 mL) was added sodium hydride (232 mg, 5.79 mmol, 60% purity, 20.0 eq) at 0° C., the mixture was stirred at 0° C. for 0.5 h, then tert-butyl (S)-4-fluoro-2-((methylsulfonyl)methyl)indoline-1-carboxylate (100 mg, 290 mol, 1.00 eq) was added, the mixture was stirred at 50° C. for 11.5 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to afford tert-butyl (S)-4-fluoro-2-((2,2,2-trifluoroethoxy)methyl)indoline-1-carboxylate (100 mg, crude) as yellow oil. ¹H NMR (400 MHz, DMSO-d₆) δ=7.51-7.29 (m, 1H), 7.24-7.16 (m, 1H), 6.78 (t, J=8.8 Hz, 1H), 4.60 (tdd, J=3.2, 6.6, 9.6 Hz, 1H), 4.16-4.03 (m, 2H), 3.80-3.74 (m, 1H), 3.74-3.68 (m, 1H), 3.28 (br d, J=10.6 Hz, 1H), 2.93 (dd, J=2.6, 16.6 Hz, 1H), 1.51 (s, 9H). MS (ESI) m/z 250.1 [M−56+H]⁺

Step 3. To a solution of tert-butyl (S)-4-fluoro-2-((2,2,2-trifluoroethoxy)methyl)indoline-1-carboxylate (100 mg, 286 mol, 1.00 eq) in dichloromethane (2.00 mL) was added trifluoroacetic acid (61 mg, 5.38 mmol, 400 μL, 18.8 eq), the mixture was stirred at 20° C. for 1 h. The reaction mixture was concentrated under reduced pressure to afford (S)-4-fluoro-2-((2,2,2-trifluoroethoxy)methyl)indoline (70.0 mg, crude) as brown oil. MS (ESI) m/z 249.9 [M+H]⁺

Step 4. To a solution of (S)-4-fluoro-2-((2,2,2-trifluoroethoxy)methyl)indoline (70.0 mg, 281 mol, 1.00 eq) and phenyl (2-(2,6-dioxopiperidin-3-yl)-1-oxoisoindolin-5-yl)carbamate (160 mg, 421 mol, 1.50 eq) in dimethylformamide (1.00 mL) was added triethylamine (85.3 mg, 843 mol, 117 μL, 3.00 eq), the mixture was stirred at 50° C. for 12 h. The mixture was filtered to give filtrate. The filtrate was purified by Prep-HPLC (column: Phenomenex luna C18 150*25 mm*10 um; mobile phase: [water (FA)-ACN]; gradient:38%-68% B over 10 min) and lyophilized to afford (2S)—N-(2-(2,6-dioxopiperidin-3-yl)-1-oxoisoindolin-5-yl)-4-fluoro-2-((2,2,2-trifluoroethoxy)methyl)indoline-1-carboxamide (22.31 mg, 41.3 mol, 14% yield, 99% purity) as a gray solid. ¹H NMR (400 MHz, DMSO-d₆) δ=10.97 (br s, 1H), 9.05 (br s, 1H), 7.84 (s, 1H), 7.70-7.61 (m, 2H), 7.60-7.54 (m, 1H), 7.25-7.17 (m, 1H), 6.83-6.76 (m, 1H), 5.17-5.05 (m, 2H), 4.41 (s, 1H), 4.35-4.26 (m, 1H), 4.18-4.08 (m, 2H), 3.82-3.68 (m, 2H), 3.41-3.36 (m, 1H), 3.01-2.94 (m, 1H), 2.94-2.86 (m, 1H), 2.62-2.58 (m, 1H), 2.43-2.34 (m, 1H), 2.06-1.95 (m, 1H). MS (ESI) m/z 535.2 [M+H]⁺

### Example 68. Synthesis of Compound 308

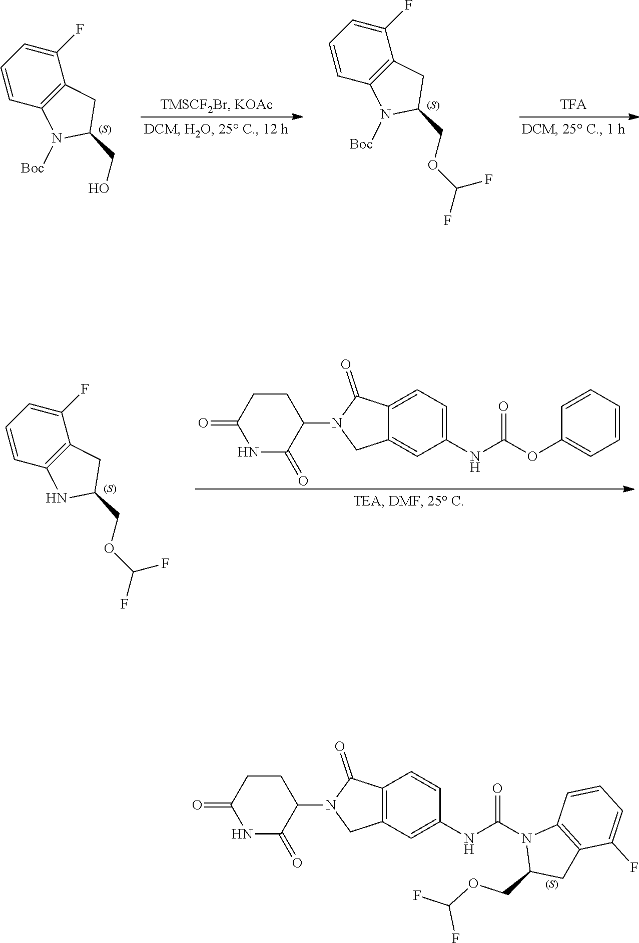

Step 1. To a solution of (S)-tert-butyl 4-fluoro-2-(hydroxymethyl)indoline-1-carboxylate (100 mg, 374 mol, 1.00 eq) in dichloromethane (3.00 mL) and water (3.00 mL) was added (bromodifluoromethyl)trimethylsilane (759 mg, 3.74 mmol, 10.0 eq) and potassium acetate (367 mg, 3.74 mmol, 10.0 eq), the mixture was stirred at 50° C. for 3 h. The reaction mixture was poured into water (40 mL) and extracted with ethyl acetate (3×40 mL). The combined organic phase was washed with brine (80 mL), dried over anhydrous sodium sulfate, filtered and concentrated in vacuum to give a residue. The residue was purified by silica gel chromatography silica gel chromatography (Petroleum ether/Ethyl acetate=1/0 to 10/1) to afford (S)-tert-butyl 2-((difluoromethoxy)methyl)-4-fluoroindoline-1-carboxylate (70.0 mg, 220 mol, 58% yield) as a yellow solid. ¹H NMR (400 MHz, CDCl₃) δ=7.60-7.32 (m, 1H), 7.12-7.02 (m, 1H), 6.60 (t, J=8.6 Hz, 1H), 6.35-5.89 (m, 1H), 4.60 (br s, 1H), 4.00 (dd, J=3.5, 9.9 Hz, 1H), 3.92-3.75 (m, 1H), 3.21 (dd, J=9.9, 16.7 Hz, 1H), 3.01 (dd, J=2.8, 16.6 Hz, 1H), 1.50 (s, 9H).

Step 2. To a solution of (S)-2-((difluoromethoxy)methyl)-4-fluoroindoline (60.0 mg, 189 mol, 1.00 eq) in dichloromethane (2.00 mL) was added trifluoroacetic acid (614 mg, 5.38 mmol, 400 μL, 28.5 eq), the mixture was stirred at 25° C. for 1 h. The reaction mixture was concentrated to give (S)-2-((difluoromethoxy)methyl)-4-fluoroindoline (40.0 mg, crude) as yellow oil. MS (ESI) m/z 218.1 [M+H]⁺

Step 3. To a solution of (S)-2-((difluoromethoxy)methyl)-4-fluoroindoline (40.0 mg, 184 mol, 1.00 eq) and triethylamine (74.5 mg, 736 mol, 102 μL, 4.00 eq) in dimethylformamide (2.00 mL) was added phenyl (2-(2,6-dioxopiperidin-3-yl)-1-oxoisoindolin-5-yl)carbamate (69.9 mg, 184 mol, 1.00 eq), the mixture was stirred at 50° C. for 2 h. The reaction mixture was filtered to give a filtrate. The filtrate was purified by Prep-HPLC (column: Phenomenex luna C18 150*25 mm*10 um; mobile phase: [water (FA)-ACN]; gradient:33%-63% B over 10 min) to afford (2S)-2-((difluoromethoxy)methyl)-N-(2-(2,6-dioxopiperidin-3-yl)-1-oxoisoindolin-5-yl)-4-fluoroindoline-1-carboxamide (26.93 mg, 53.6 mol, 29% yield) as a white solid.

¹H NMR (400 MHz, CDCl₃) δ=8.02 (s, 1H), 7.93 (d, J=11.6 Hz, 1H), 7.83 (d, J=8.1 Hz, 1H), 7.77 (d, J=7.3 Hz, 1H), 7.46 (d, J=8.1 Hz, 1H), 7.28-7.14 (m, 2H), 6.79 (t, J=8.4 Hz, 1H), 6.58-6.12 (m, 1H), 5.22 (ddd, J=5.2, 11.4, 13.1 Hz, 1H), 4.92-4.81 (m, 1H), 4.54-4.45 (m, 1H), 4.40-4.30 (m, 1H), 4.17-4.02 (m, 2H), 3.49 (br dd, J=10.3, 16.4 Hz, 1H), 2.99-2.80 (m, 3H), 2.38 (tq, J=5.1, 13.0 Hz, 1H), 2.30-2.18 (m, 1H).

¹H NMR (400 MHz, DMSO-d₆) δ=11.06-10.87 (m, 1H), 9.14 (br dd, J=3.9, 7.5 Hz, 1H), 7.86 (br d, J=11.4 Hz, 1H), 7.75-7.53 (m, 3H), 7.31-7.15 (m, 1H), 6.94-6.5 (m, 1H), 6.85-6.75 (m, 1H), 5.27-5.04 (m, 2H), 4.54-4.39 (m, 1H), 4.38-4.25 (m, 1H), 4.12-3.93 (m, 2H), 3.50-3.41 (m, 1H), 3.09-2.93 (m, 2H), 2.69-2.60 (m, 1H), 2.42 (br dd, J=4.4, 13.1 Hz, 1H), 2.02 (br d, J=0.9 Hz, 1H). MS (ESI) m/z 503.2 [M+H]⁺

### Example 69. Synthesis of Compound 299

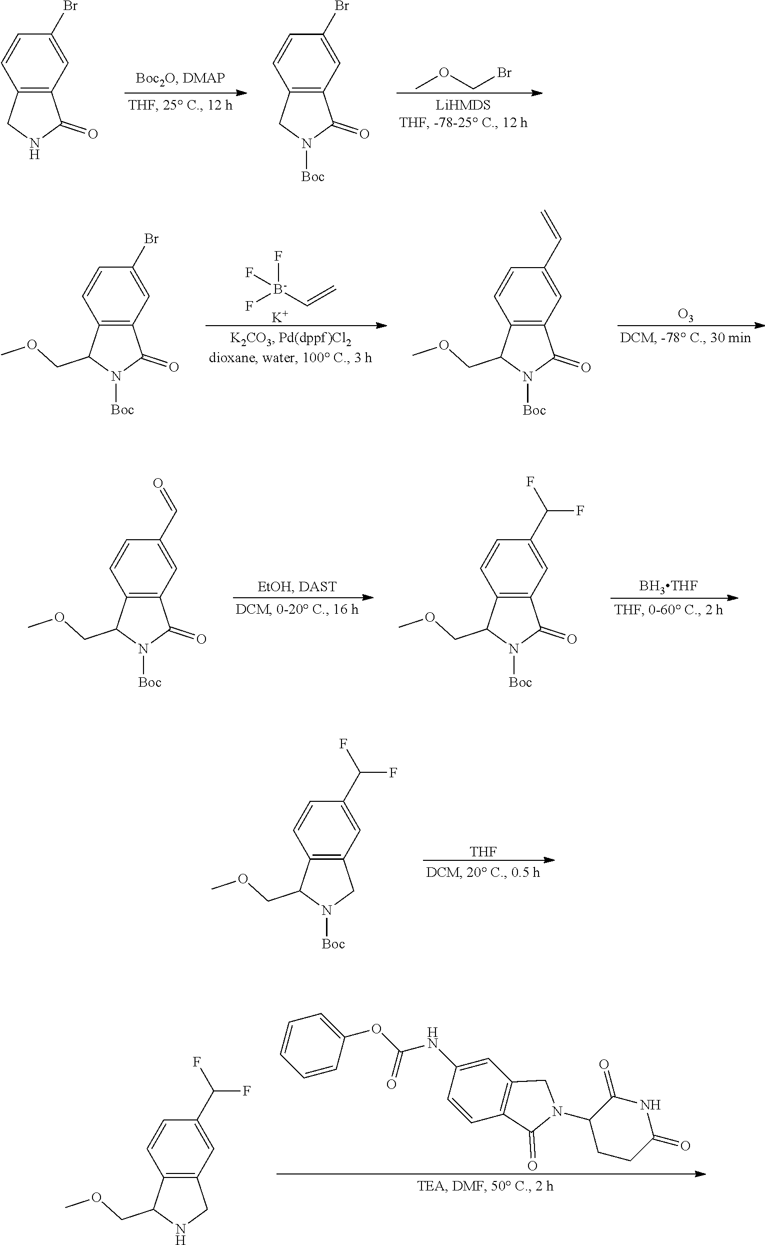

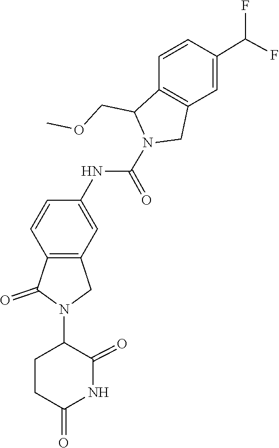 [SMILES: COCC1c2ccc(C(F)F)cc2CN1C(=O)Nc1ccc2c(c1)CN(C1CCC(=O)NC1=O)C2=O]

Step 1. To a solution of 6-bromoisoindolin-1-one (5.00 g, 23.6 mmol, 1.00 eq) in tetrahydrofuran (50.0 mL) was added N,N-dimethylpyridin-4-amine (288 mg, 2.36 mmol, 0.100 eq) and di-tert-butyl dicarbonate (12.9 g, 59.0 mmol, 2.50 eq). Then the reaction mixture was stirred at 25° C. for 12 hours. The reaction mixture was diluted with water (100 mL) and extracted with ethyl acetate (3×100 mL). The combined organic layers were washed with brine (3×20.0 mL), dried over sodium sulfate, filtered and concentrated under reduced pressure to give a residue. The residue was purified by column chromatography (silicon dioxide, petroleum ether/ethyl acetate=20/1 to 2/1) to get tert-butyl 6-bromo-1-oxoisoindoline-2-carboxylate (6.30 g, 18.6 mmol, 79% yield) as a white solid. MS (ESI) m/z 258.0 [M+H−56]⁺

Step 2. To a solution of tert-butyl 6-bromo-1-oxoisoindoline-2-carboxylate (6.00 g, 19.2 mmol, 1.00 eq) in tetrahydrofuran (100 mL) was dropwise added lithium bis(trimethylsilyl)amide (1.00 M, 23.1 mL, 1.20 eq) at −78° C. under nitrogen atmosphere. Then the reaction mixture was stirred at −78° C. for 1 hours. Then the reaction mixture was dropwise added bromo(methoxy)methane (4.80 g, 38.4 mmol, 2.00 eq) at −78° C. under nitrogen atmosphere. Then the reaction mixture was stirred at 25° C. for 11 hours under nitrogen atmosphere. The reaction mixture was quenched by addition ammonium chloride (30.0 mL) at 0° C. and extracted with ethyl acetate (3×100 mL). The combined organic layers were washed with brine (3×20.0 mL), dried over anhydrous sodium sulfate, filtered and concentrated under reduced pressure to give a residue. The residue was purified by column chromatography (silicon dioxide, petroleum ether/ethyl acetate=20/1 to 2/1) to get tert-butyl 5-bromo-1-(methoxymethyl)-3-oxoisoindoline-2-carboxylate (6.40 g, 18.0 mmol, 93% yield) as a white solid. ¹H NMR (400 MHz, DMSO-d₆) δ=7.96-7.83 (m, 2H), 7.65 (d, J=8.0 Hz, 1H), 5.20-5.15 (m, 1H), 3.97-3.91 (m, 1H), 3.90-3.84 (m, 1H), 3.18-3.14 (m, 3H), 1.52 (s, 9H).

Step 3. To a mixture of tert-butyl 5-bromo-1-(methoxymethyl)-3-oxo-isoindoline-2-carboxylate (4.00 g, 11.2 mmol, 1.00 eq), potassium; trifluoro(vinyl)boranuide (1.80 g, 13.5 mmol, 1.20 eq), potassium carbonate (3.10 g, 22.5 mmol, 2.00 eq), and [1,1′-bis(diphenylphosphino)ferrocene]dichloropalladium(II) (822 mg, 1.12 mmol, 0.100 eq) in dioxane (50.0 mL) and water (10.0 mL) was degassed and purged with nitrogen for 3 times, and then the mixture was stirred at 100° C. for 3 hours under nitrogen atmosphere. The reaction mixture was partitioned between ethyl acetate (100 mL) and water (70.0 mL). The organic phase was separated, washed with brine (2×40.0 mL), dried over anhydrous sodium sulfate, filtered and concentrated under reduced pressure to give a residue. The residue was purified by column chromatography (silicon dioxide, petroleum ether/ethyl acetate=1/0 to 5/1) to get tert-butyl 1-(methoxymethyl)-3-oxo-5-vinylisoindoline-2-carboxylate (2.50 g, 7.91 mmol, 70% yield) as a yellow solid. ¹H NMR (400 MHz, DMSO-d₆) δ=7.88-7.77 (m, 2H), 7.65 (d, J=8.0 Hz, 1H), 6.86 (dd, J=11.2, 17.6 Hz, 1H), 5.97 (d, J=17.6 Hz, 1H), 5.36 (d, J=11.2 Hz, 1H), 5.20-5.15 (m, 1H), 3.97-3.92 (m, 1H), 3.90-3.84 (m, 1H), 3.15 (s, 3H), 1.53 (s, 9H).

Step 4. To a solution of tert-butyl 1-(methoxymethyl)-3-oxo-5-vinylisoindoline-2-carboxylate (1.00 g, 3.30 mmol, 1.00 eq) in dichloromethane (40.0 mL) was cooled to −78° C. and a stream of ozone was passed through it for 30 minutes. At this time, ozone gas was bubbled into the reaction mixture until the color of the reaction mixture turned to blue. After completion of the reaction, the mixture was purged with oxygen gas for 30 min. The reaction mixture was concentrated to get a residue. The residue was purified by column chromatography (silicon dioxide, petroleum ether/ethyl acetate=1/0 to 3/1) to get tert-butyl 5-formyl-1-(methoxymethyl)-3-oxoisoindoline-2-carboxylate (560 mg, 1.78 mmol, 54% yield) as a white solid. MS (ESI) m/z 206.3 [M+H−100]⁺

Step 5. To a solution of tert-butyl 5-formyl-1-(methoxymethyl)-3-oxoisoindoline-2-carboxylate (330 mg, 1.08 mmol, 1.00 eq) in dichloromethane (10.0 mL) was added (diethylamino)sulfur trifluoride (871 mg, 5.40 mmol, 5.00 eq) and ethanol (4.98 mg, 108 mol, 0.100 eq) at 0° C. The mixture was stirred at 20° C. for 16 hours. The reaction mixture was quenched by addition water (5.00 mL) at 0° C., and then diluted with ethyl acetate (20.0 mL) and extracted with ethyl acetate (2×10.0 mL). The combined organic layers were washed with brine (2×10.0 mL), dried over anhydrous sodium sulfate, filtered and concentrated under reduced pressure to give tert-butyl 5-(difluoromethyl)-1-(methoxymethyl)-3-oxoisoindoline-2-carboxylate (280 mg, 650 mol, 60% yield) as a yellow solid. MS (ESI) m/z 228.2 [M+H−100]⁺

Step 6. To a solution of tert-butyl 5-(difluoromethyl)-1-(methoxymethyl)-3-oxoisoindoline-2-carboxylate (200 mg, 611 mol, 1.00 eq) in tetrahydrofuran (5.00 mL) was added borane-tetrahydrofuran complex (1.00 M, 6.11 mL, 10.0 eq) at 0° C. The mixture was stirred at 60° C. for 2 hours. The reaction mixture was quenched by addition methanol 5.00 mL at 0° C., and then concentrated under reduced pressure to give tert-butyl 5-(difluoromethyl)-1-(methoxymethyl)isoindoline-2-carboxylate (180 mg, 396 mol, 65% yield) as pink oil. MS (ESI) m/z 214.3 [M+H−100]⁺

Step 7. To a solution of tert-butyl 5-(difluoromethyl)-1-(methoxymethyl)isoindoline-2-carboxylate (150 mg, 479 mol, 1.00 eq) in dichloromethane (5.00 mL) was added trifluoroacetic acid (1.54 g, 13.5 mmol, 1.00 mL, 28.1 eq). The mixture was stirred at 20° C. for 30 minutes. The reaction mixture was concentrated to get 5-(difluoromethyl)-1-(methoxymethyl)isoindoline (100 mg, 352 mol, 73% yield) as yellow oil. MS (ESI) m/z 214.3 [M+H]⁺

Step 8. To a solution of 5-(difluoromethyl)-1-(methoxymethyl)isoindoline (100 mg, 469 mol, 1.00 eq) and phenyl (2-(2,6-dioxopiperidin-3-yl)-1-oxoisoindolin-5-yl)carbamate (177 mg, 468 mol, 1.00 eq) in dimethyl formamide (3.00 mL) was added triethylamine (190 mg, 1.88 mmol, 261 μL, 4.00 eq). The mixture was stirred at 50° C. for 2 hours. The mixture was filtered and the filtrate was collected. The filtrate was purified by prep-HPLC (column: phenomenex luna C18 150*25 mm*10 um; mobile phase: [water (formic acid)-acetonitrile]; gradient:25%-55% B over 15 min) to afford 5-(difluoromethyl)-N-(2-(2,6-dioxopiperidin-3-yl)-1-oxoisoindolin-5-yl)-1-(methoxymethyl)isoindoline-2-carboxamide (92.9 mg, 183 mol, 39% yield) as an off-white solid. ¹H NMR (400 MHz, DMSO-d₆) δ=10.97 (s, 1H), 8.90 (s, 1H), 7.88 (s, 1H), 7.67-7.39 (m, 5H), 7.22-6.90 (m, 1H), 5.40 (br s, 1H), 5.08 (dd, J=5.2, 13.2 Hz, 1H), 4.98-4.88 (m, 1H), 4.84-4.76 (m, 1H), 4.47-4.36 (m, 1H), 4.33-4.24 (m, 1H), 3.84-3.70 (m, 2H), 3.25 (s, 3H), 3.00-2.83 (m, 1H), 2.60 (br dd, J=2.0, 15.2 Hz, 1H), 2.38 (dq, J=4.4, 13.2 Hz, 1H), 2.05-1.90 (m, 1H). MS (ESI) m/z 499.3 [M+H]⁺

### Example 70. Synthesis of Compound 304

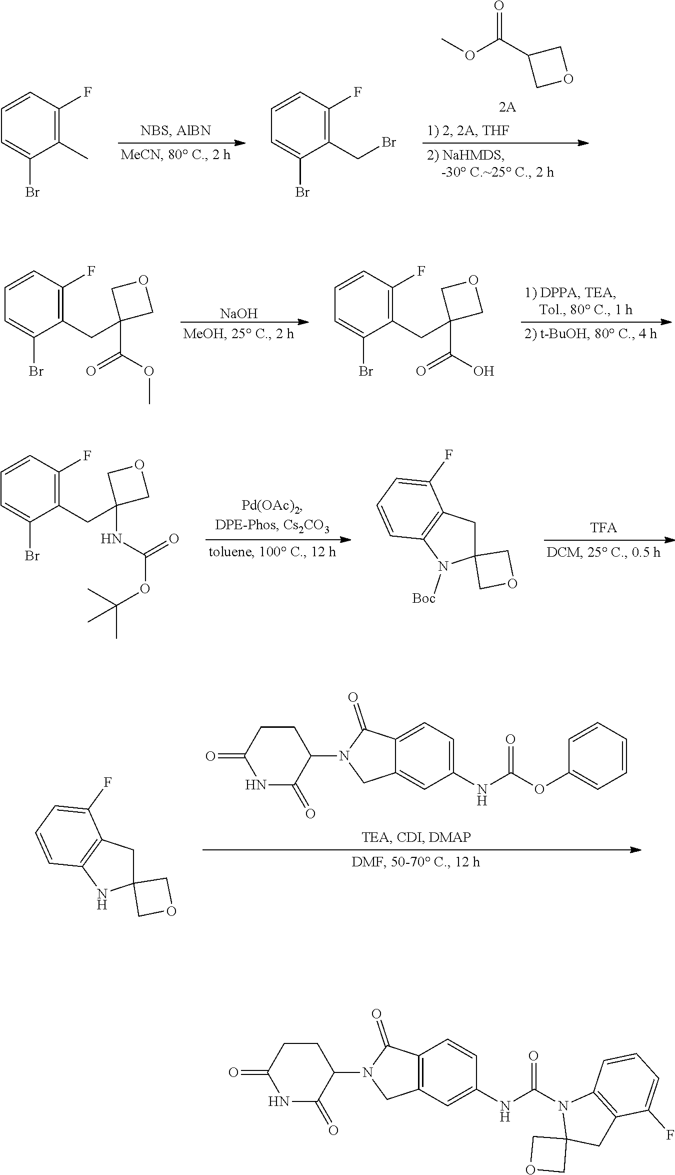

Step 1. To a solution of 1-bromo-3-fluoro-2-methyl-benzene (5.00 g, 26.4 mmol, 1.00 eq), 2-(2-cyanopropan-2-yldiazenyl)-2-methylpropanenitrile (868 mg, 5.29 mmol, 0.200 eq) in acetonitrile (10.0 mL) was added 1-bromopyrrolidine-2,5-dione (5.65 g, 31.7 mmol, 1.20 eq). The mixture was stirred at 80° C. for 2 h. The mixture was concentrated in vacuum to get a residue. The residue was purified by column chromatography (SiO₂, Petroleum ether/Ethyl acetate=1/0) to give 1-bromo-2-(bromomethyl)-3-fluorobenzene (4.80 g, 17.9 mmol, 68% yield) as colorless oil. ¹H NMR (400 MHz, CDCl₃) δ=7.40 (d, J=8.0 Hz, 1H), 7.19 (td, J=7.2, 7.8 Hz, 1H), 7.10-7.02 (m, 1H), 4.66 (s, 2H).

Step 2. To a solution of methyl oxetane-3-carboxylate (370 mg, 3.19 mmol, 1.00 eq), 1-bromo-2-(bromomethyl)-3-fluoro-benzene (1.02 g, 3.82 mmol, 1.20 eq) in tetrahydrofuran (10.0 mL) was added sodium bis(trimethylsilyl)amide (1 M, 4.14 mL, 1.30 eq) at −30° C. under nitrogen atmosphere. The mixture was stirred at −30° C. for 15 min and warmed to 25° C. for 2 h. The reaction mixture was quenched with saturated ammonium chloride (30 mL), extracted with ethyl acetate (3×50 mL). The combined organic phase was washed with brine (30 mL), dried over anhydrous sodium sulfate, filtered and concentrated in vacuum to give a residue. The residue was purified by silica gel chromatograph (SiO₂, Petroleum ether/Ethyl acetate=1/0 to 10/1) to get methyl 3-(2-bromo-6-fluorobenzyl)oxetane-3-carboxylate (700 mg, 2.31 mmol, 72% yield) as colorless oil. ¹H NMR (400 MHz, CDCl₃) δ=7.42 (d, J=8.0 Hz, 1H), 7.15 (dt, J=6.0, 8.0 Hz, 1H), 7.08-7.02 (m, 1H), 4.87 (d, J=6.4 Hz, 2H), 4.70 (d, J=6.4 Hz, 2H), 3.77 (s, 3H), 3.46 (d, J=2.4 Hz, 2H).

Step 3. To a solution of methyl 3-(2-bromo-6-fluorobenzyl)oxetane-3-carboxylate (700 mg, 2.31 mmol, 1.00 eq) in methanol (5.00 mL) and water (1.00 mL) was added sodium hydroxide (277 mg, 6.93 mmol, 3.00 eq). The mixture was stirred at 25° C. for 2 h. The mixture was concentrated in vacuum and extracted with ethyl acetate (2×30 mL). The aqueous phase was acidified with aqueous 1 M HCl till pH=5-6 and extracted with ethyl acetate (3×30 mL). The combined organic layers were washed with brine (20 mL), dried over anhydrous sodium sulfate, filtered and concentrated under reduced pressure to get 3-(2-bromo-6-fluorobenzyl)oxetane-3-carboxylic acid (470 mg, 1.63 mmol, 70% yield) as a yellow solid. ¹H NMR (400 MHz, DMSO-d₆) δ=12.96 (br s, 1H), 7.56-7.51 (m, 1H), 7.34-7.24 (m, 2H), 4.68 (d, J=6.4 Hz, 2H), 4.50 (d, J=6.4 Hz, 2H), 3.34 (br s, 2H). MS (ESI) m/z 311.1 [M+Na]⁺

Step 4. To a solution of 3-(2-bromo-6-fluorobenzyl)oxetane-3-carboxylic acid (250 mg, 865 mol, 1.00 eq), 4A MS and TEA (96.2 mg, 951 mol, 132 μL, 1.10 eq) in toluene (4.00 mL) was added diphenyl phosphoryl azide (238 mg, 865 mol, 186 μL, 1.00 eq) dropwise at 0° C. The mixture was stirred at 80° C. for 1 h, then 2-methylpropan-2-ol (6.00 mL) was added to the mixture. The mixture was stirred at 80° C. for 4 h. The mixture was filtered and concentrated in vacuum. The mixture was purified by Prep-TLC (Petroleum ether/Ethyl acetate=5/1) to get tert-butyl (3-(2-bromo-6-fluorobenzyl)oxetan-3-yl)carbamate (220 mg, 610 mol, 70% yield) as a white solid. ¹H NMR (400 MHz, DMSO-d₆) δ=7.50 (br s, 1H), 7.48 (br d, J=2.8 Hz, 1H), 7.29-7.22 (m, 2H), 4.53-4.48 (m, 2H), 4.47-4.42 (m, 2H), 3.40 (d, J=1.6 Hz, 2H), 1.37 (s, 9H).

Step 5. To a solution of tert-butyl (3-(2-bromo-6-fluorobenzyl)oxetan-3-yl)carbamate (220 mg, 610 mol, 1.00 eq), (2-(2-diphenylphosphanylphenoxy)phenyl)-diphenyl-phosphane (65.8 mg, 122 mol, 0.200 eq), cesium carbonate (398 mg, 1.22 mmol, 2.00 eq) in toluene (5.00 mL) was added palladium acetate (13.7 mg, 61.1 mol, 0.100 eq) under nitrogen atmosphere. The mixture was stirred at 100° C. for 12 h. The reaction mixture was concentrated in vacuum to give a residue. The residue was purified by Prep-TLC (SiO₂, Petroleum ether/Ethyl acetate=5:1) to get tert-butyl 4-fluorospiro[indoline-2,3′-oxetane]-1-carboxylate (150 mg, 537 mol, 88% yield) as a white solid. ¹H NMR (400 MHz, DMSO-d₆) δ=7.51 (br d, J=7.6 Hz, 1H), 7.24-7.16 (m, 1H), 6.79 (t, J=8.4 Hz, 1H), 5.29 (d, J=6.4 Hz, 2H), 4.55 (d, J=6.4 Hz, 2H), 3.68 (s, 2H), 1.59 (s, 9H).

Step 6. To a solution of tert-butyl 4-fluorospiro[indoline-2,3′-oxetane]-1-carboxylate (70 mg, 251 mol, 1.00 eq) in dichloromethane (1.00 mL) and trifluoroacetic acid (0.100 mL) was stirred at 25° C. for 30 min. The reaction mixture was concentrated to give 4-fluorospiro[indoline-2,3′-oxetane](70 mg, crude) as yellow oil. MS (ESI) m/z 180.0 [M+H]⁺

Step 7. To a solution of phenyl (2-(2,6-dioxopiperidin-3-yl)-1-oxoisoindolin-5-yl)carbamate (136 mg, 358 mol, 1.50 eq), 4-fluorospiro[indoline-2,3′-oxetane](70.0 mg, 238 mol, 1.00 eq, TFA) in N,N-dimethyl formamide (2.00 mL) was added 4-dimethylaminopyridin (2.92 mg, 23.8 mol, 0.100 eq) and triethylamine (72.5 mg, 716 mol, 99.7 μL, 3.00 eq). The mixture was stirred at 50° C. for 2 h. Then 1,1′-carbonyldiimidazole (387 mg, 2.39 mmol, 10.0 eq) was added to the mixture and stirred at 70° C. for 10 h. The reaction mixture was adjusted to pH=5-6 with formic acid and filtered to give a filtrate. The filtrate was purified by Prep-HPLC (column: Phenomenex luna C18 150*25 mm*10 um; mobile phase: [water (formic acid)-acetonitrile]; gradient: 25%-55% B over 10 min) and lyophilized to afford N-(2-(2,6-dioxopiperidin-3-yl)-1-oxoisoindolin-5-yl)-4-fluorospiro[indoline-2,3′-oxetane]-1-carboxamide (9.88 mg, 20.8 mol, 9% yield, 98% purity) as a white solid. ¹H NMR (400 MHz, DMSO-d₆) δ=10.99 (s, 1H), 9.98 (s, 1H), 7.88 (s, 1H), 7.69 (d, J=8.4 Hz, 1H), 7.58 (d, J=8.4 Hz, 1H), 7.29-7.21 (m, 1H), 7.12 (d, J=8.0 Hz, 1H), 6.81 (t, J=8.4 Hz, 1H), 5.34 (d, J=6.4 Hz, 2H), 5.11 (dd, J=5.2, 13.2 Hz, 1H), 4.67 (d, J=6.4 Hz, 2H), 4.49-4.41 (m, 1H), 4.36-4.29 (m, 1H), 3.61 (s, 2H), 2.99-2.87 (m, 1H), 2.61 (br d, J=17.0 Hz, 1H), 2.42-2.38 (m, 1H), 2.06-1.96 (m, 1H). MS (ESI) m/z 465.0 [M+H]⁺

### Example 71. Synthesis of Compound 298

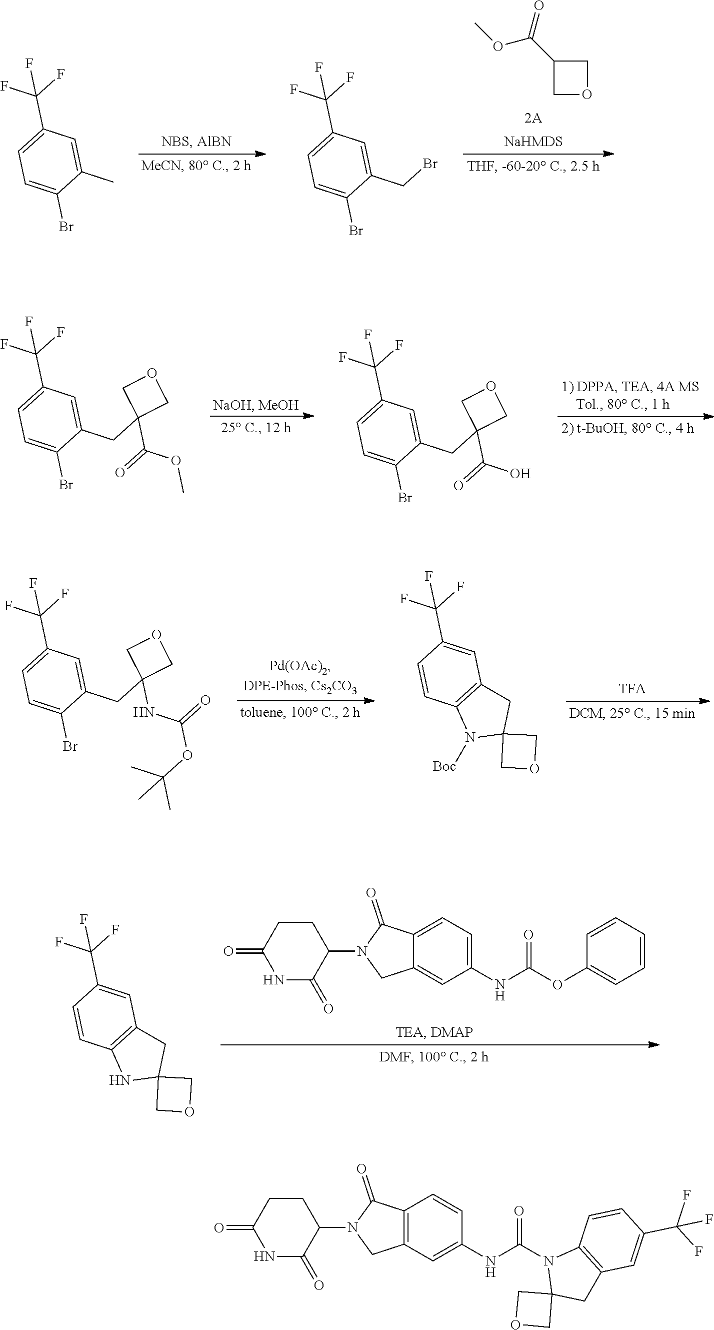

Step 1. To a solution of 1-bromo-2-methyl-4-(trifluoromethyl)benzene (5.00 g, 20.9 mmol, 1.00 eq) in acetonitrile (50.0 mL) was added N-bromosuccinimide (7.45 g, 41.8 mmol, 2.00 eq) and 2,2′-azobis(isobutyronitrile) (343 mg, 2.09 mmol, 0.100 eq). Then the reaction mixture was stirred at 80° C. for 2 h under nitrogen atmosphere. The reaction mixture was concentrated in vacuum to get 1-bromo-2-(bromomethyl)-4-(trifluoromethyl)benzene (5.20 g, 16.3 mmol, 78% yield) as colorless oil. ¹H NMR (400 MHz, DMSO-d₆) δ=8.01 (d, J=8.4 Hz, 1H), 7.92 (d, J=8.4 Hz, 1H), 7.74 (dd, J=2.4, 8.4 Hz, 1H), 4.91 (s, 2H).

Step 2. To a solution of 1-bromo-2-(bromomethyl)-4-(trifluoromethyl)benzene (2.63 g, 8.27 mmol, 1.20 eq) and methyl oxetane-3-carboxylate (800 mg, 6.89 mmol, 1.00 eq) in tetrahydrofuran (4.00 mL) was added sodium hexamethyldisilazane (1 M, 8.27 mL, 1.20 eq) dropwise at −60° C. under nitrogen atmosphere. Then the reaction mixture was stirred at −60° C. for 0.5 h. Then the reaction mixture was stirred at 20° C. for 2 h. The reaction mixture was quenched by addition saturated ammonium chloride (100 mL) at 0° C., and extracted with ethyl acetate (3×60 mL). The combined organic layers were washed with brine (10 mL), dried over sodium sulfate, filtered and concentrated under reduced pressure to give a residue. The residue was purified by column chromatography (SiO₂, Petroleum ether/Ethyl acetate=1/0 to 0/1) and concentrated in vacuum to get ethyl 2-oxo-3-(2,2,2-trifluoro-N-phenylacetimidamido)propanoate (1.00 g, 2.55 mmol, 18% yield, 90% purity) as yellow oil.

¹H NMR (400 MHz, CDCl₃) δ=7.64 (d, J=8.4 Hz, 1H), 7.33-7.28 (m, 2H), 4.88 (d, J=6.4 Hz, 2H), 4.60 (d, J=6.4 Hz, 2H), 3.68 (s, 3H), 3.49 (s, 2H). (¹H NMR came from pilot run)

Step 3. To a solution of ethyl 2-oxo-3-(2,2,2-trifluoro-N-phenylacetimidamido)propanoate (1.00 g, 2.83 mmol, 1.00 eq) in methanol (10.0 mL) and water (1.00 mL) was added sodium hydroxide (339 mg, 8.50 mmol, 3.00 eq). Then the reaction mixture was stirred at 25° C. for 12 h. The reaction mixture was diluted with water (100 mL) and extracted with ethyl acetate (3×50 mL). The aqueous layers were adjusted pH to 5-6 by hydrochloric acid (1 M, 100 mL) and extracted with ethyl acetate (3×50 mL). The combined organic layers were washed with brine (3×20 mL), dried over sodium sulfate, filtered and concentrated under reduced pressure to give 3-(2-bromo-5-(trifluoromethyl)benzyl)oxetane-3-carboxylic acid (600 mg, 1.77 mmol, 62% yield) as yellow oil. ¹H NMR (400 MHz, CDCl₃) δ=7.66 (d, J=8.4 Hz, 1H), 7.35-7.28 (m, 1H), 7.25 (s, 1H), 4.93 (d, J=6.4 Hz, 2H), 4.62 (d, J=6.4 Hz, 2H), 3.50 (s, 2H).

Step 4. To a solution of 3-(2-bromo-5-(trifluoromethyl)benzyl)oxetane-3-carboxylic acid (180 mg, 530 mol, 1.00 eq) and triethylamine (161 mg, 1.59 mmol, 221 μL, 3.00 eq) in toluene (5.00 mL) was added diphenyl phosphoryl azide (175 mg, 636 mol, 137 L, 1.20 eq) dropwise at 0° C. under nitrogen atmosphere. Then the reaction mixture was stirred at 80° C. for 1 h. Then the reaction mixture was added tert-butyl alcohol (8.00 mL) at 25° C. Then the reaction mixture was stirred at 80° C. for 12 h. The reaction mixture was concentrated in vacuum. The residue was purified by Prep-TLC (SiO₂, Petroleum ether: Ethyl acetate=5:1) to get tert-butyl (3-(2-bromo-5-(trifluoromethyl)benzyl)oxetan-3-yl)carbamate (400 mg, 877 mol, 55% yield, 90% purity) as a white solid. ¹H NMR (400 MHz, CDCl₃) δ=7.63 (d, J=8.4 Hz, 1H), 7.35-7.27 (m, 2H), 4.68 (br d, J=6.0 Hz, 2H), 4.53 (d, J=6.4 Hz, 2H), 3.55 (s, 2H), 1.39 (s, 9H).

Step 5. To a solution of tert-butyl (3-(2-bromo-5-(trifluoromethyl)benzyl)oxetan-3-yl)carbamate (390 mg, 950 mol, 1.00 eq) and cesium carbonate (929 mg, 2.85 mmol, 3.00 eq) in toluene (5.00 mL) was added [2-(2-diphenylphosphanylphenoxy)phenyl]-diphenyl-phosphane (102 mg, 190 mol, 0.200 eq) and palladium acetate (21.3 mg, 95.0 mol, 0.100 eq). Then the reaction mixture was stirred at 100° C. for 2 h under nitrogen atmosphere. The reaction mixture was diluted with water (50 mL) and extracted with ethyl acetate (3×30 mL). The combined organic layers were washed with brine (3×10 mL), dried over sodium sulfate, filtered and concentrated under reduced pressure to give a residue. The residue was purified by Prep-TLC (SiO₂, Petroleum ether:Ethyl acetate=5:1) and concentrated in vacuum to get tert-butyl 5-(trifluoromethyl)spiro[indoline-2,3′-oxetane]-1-carboxylate (250 mg, 759 mol, 79% yield) as a white solid. ¹H NMR (400 MHz, DMSO-d₆) δ=7.83 (br d, J=8.4 Hz, 1H), 7.55-7.51 (m, 2H), 5.30 (d, J=6.4 Hz, 2H), 4.54 (d, J=6.4 Hz, 2H), 3.73 (s, 2H), 1.61 (s, 9H).

Step 6. To a solution of tert-butyl 5-(trifluoromethyl)spiro[indoline-2,3′-oxetane]-1-carboxylate (150 mg, 455 mol, 1.00 eq) in trifluoroacetic acid (0.400 mL) and dichloromethane (2.00 mL) was stirred at 25° C. for 15 min. The reaction mixture was concentrated in vacuum to give 5-(trifluoromethyl)spiro[indoline-2,3′-oxetane](120 mg, crude) as yellow oil. MS (ESI) m/z. 230.2 [M+H]⁺

Step 7. To a solution of 5-(trifluoromethyl)spiro[indoline-2,3′-oxetane](40.0 mg, 174 mol, 1.00 eq) and phenyl (2-(2,6-dioxopiperidin-3-yl)-1-oxoisoindolin-5-yl)carbamate (529 mg, 1.40 mmol, 8.00 eq) in dimethylformamide (10.0 mL) and acetonitrile (10.0 mL) was added triethylamine (52.9 mg, 523 mol, 72.8 μL, 3.00 eq), N,N-dimethylpyridin-4-amine (2.13 mg, 17.4 mol, 0.100 eq). Then the reaction mixture was stirred at 100° C. for 2 h. The reaction mixture was adjusted pH to 5-6 by formic acid (0.2 mL) and filtered. The filtrate was purified by reversed phase column chromatography (C18, 330 g; condition: water/acetonitrile=1/0 to 0/1, 0.1% formic acid) and lyophilized to afford a white solid. The white solid was purified by Prep-TLC (SiO₂, Petroleum ether:Ethyl acetate=0:1) and concentrated in vacuum. The residue was purified by reversed phase column chromatography (C18, 120 g; condition: water/acetonitrile=1/0 to 0/1, 0.1% formic acid) and lyophilized to afford N-(2-(2,6-dioxopiperidin-3-yl)-1-oxoisoindolin-5-yl)-5-(trifluoromethyl)spiro[indoline-2,3′-oxetane]-1-carboxamide (12.0 mg, 23.0 mol, 4% yield, 99% purity) as a white solid. ¹H NMR (400 MHz, DMSO-d₆) δ=10.99 (br s, 1H), 10.09 (s, 1H), 8.50 (br s, 0.5H), 7.90 (s, 1H), 7.70 (d, J=8.4 Hz, 1H), 7.63-7.52 (m, 3H), 7.42 (br d, J=8.4 Hz, 1H), 5.34 (br d, J=6.4 Hz, 2H), 5.11 (br dd, J=4.8, 12.8 Hz, 1H), 4.66 (br d, J=6.4 Hz, 2H), 4.49-4.42 (m, 1H), 4.36-4.29 (m, 1H), 3.64 (s, 2H), 2.99-2.88 (m, 1H), 2.65-2.60 (m, 1H), 2.43-2.39 (br s, 1H), 2.04-1.98 (m, 1H). MS (ESI) m/z. 515.2 [M+H]⁺

### Example 72. Synthesis of Compound 296

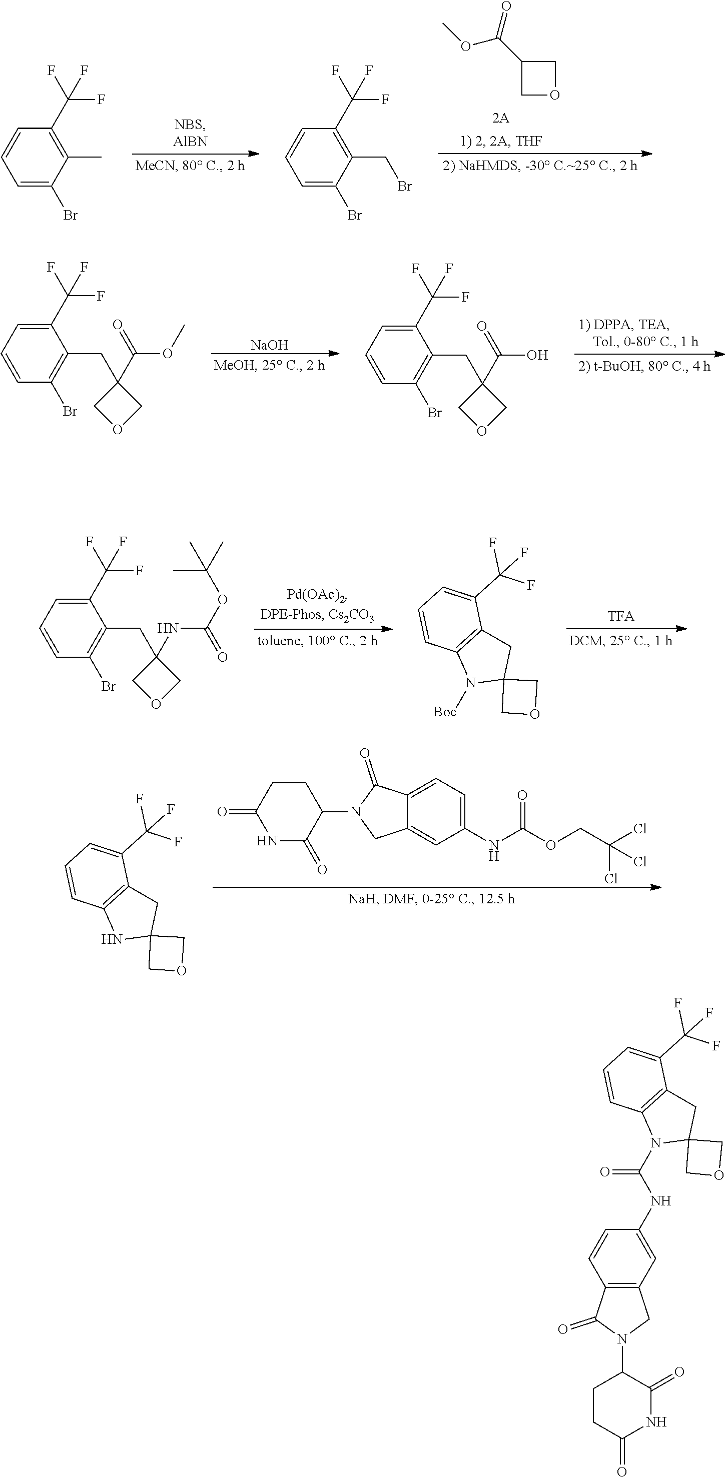

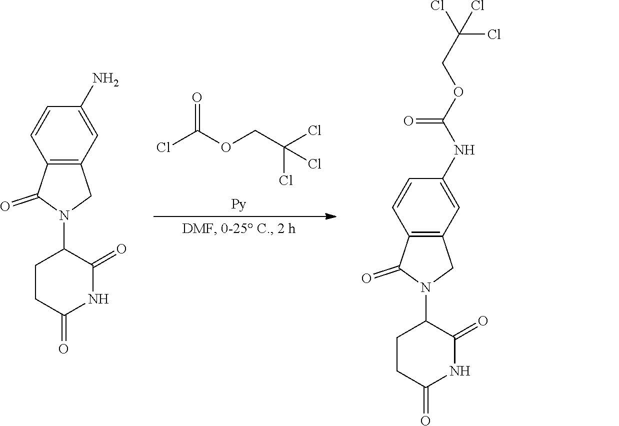 [SMILES: Nc1ccc2c(c1)CN(C1CCC(=O)NC1=O)C2=O ; O=C(Cl)OCC(Cl)(Cl)Cl ; O=C1CCC(N2Cc3cc(NC(=O)OCC(Cl)(Cl)Cl)ccc3C2=O)C(=O)N1]

Step 1. To a solution of 1-bromo-2-methyl-3e(trifluoromethyl)benzene (5.00 g, 20.9 mmol, 1.00 eq), 2-(2-cyanopropan-2-yldiazenyl)-2-methylpropanenitrile (343 mg, 2.09 mmol, 0.100 eq) in acetonitrile (15.0 mL) was added 1-bromopyrrolidine-2,5-dione (4.47 g, 25.1 mmol, 1.20 eq). The mixture was stirred at 80° C. for 2 h. The mixture was concentrated in vacuum to get a residue. The residue was purified by column chromatography (SiO₂, Petroleum ether/Ethyl acetate=1/0) to give 1-bromo-2-(bromomethyl)-3-(trifluoromethyl)benzene (4.70 g, 14.8 mmol, 71% yield) as colorless oil.

¹H NMR (400 MHz, CDCl₃) δ=7.85 (d, J=8.0 Hz, 1H), 7.68 (d, J=7.8 Hz, 1H), 7.32 (t, J=8.0 Hz, 1H), 4.77 (s, 2H).

Step 2. To a solution of 1-bromo-2-(bromomethyl)-3-(trifluoromethyl)benzene (2.00 g, 6.30 mmol, 1.20 eq), methyl oxetane-3-carboxylate (610 mg, 5.25 mmol, 1.00 eq) in tetrahydrofuran (15.0 mL) was added sodium bis(trimethylsilyl)amide (1 M, 6.83 mL, 1.30 eq) at −30° C. under nitrogen atmosphere. The reaction was warmed to 25° C. and stirred for 2 h. The reaction mixture was quenched with saturated ammonium chloride (30 mL), extracted with ethyl acetate (3×80 mL). The combined organic phase was washed with brine (50 mL), dried over anhydrous sodium sulfate, filtered and concentrated in vacuum to give a residue. The residue was purified by silica gel chromatograph (SiO₂, Petroleum ether/Ethyl acetate=1/0 to 10/1) to get methyl 3-(2-bromo-6-(trifluoromethyl)benzyl)oxetane-3-carboxylate (750 mg, 2.12 mmol, 40% yield) as colorless oil. ¹H NMR (400 MHz, CDCl₃) δ=7.85 (d, J=8.0 Hz, 1H), 7.71 (d, J=8.0 Hz, 1H), 7.33-7.28 (m, 1H), 4.78 (d, J=6.4 Hz, 2H), 4.65 (d, J=6.4 Hz, 2H), 3.82 (s, 3H), 3.52 (s, 2H).

Step 3. To a solution of methyl 3-(2-bromo-6-(trifluoromethyl)benzyl)oxetane-3-carboxylate (750 mg, 2.12 mmol, 1.00 eq) in methanol (5.00 mL) and water (1.00 mL) was added sodium hydroxide (254 mg, 6.37 mmol, 3.00 eq). The mixture was stirred at 25° C. for 2 h. The mixture was concentrated in vacuum and extracted with ethyl acetate (3×50.0 mL). The aqueous phase was acidified with aqueous 1 M hydrochloric acid solution till pH=5-6 and extracted with ethyl acetate (3×50.0 mL). The combined organic layers were washed with brine (20.0 mL), dried over anhydrous sodium sulfate, filtered and concentrated under reduced pressure to get 3-(2-bromo-6-(trifluoromethyl)benzyl)oxetane-3-carboxylic acid (520 mg, 1.53 mmol, 72% yield) as a white solid. ¹H NMR (400 MHz, DMSO-d₆) δ=13.03 (s, 1H), 8.05 (d, J=8.0 Hz, 1H), 7.84 (d, J=8.0 Hz, 1H), 7.49 (t, J=8.0 Hz, 1H), 4.60 (d, J=6.4 Hz, 2H), 4.44 (d, J=6.4 Hz, 2H), 3.40 (br s, 2H).

Step 4. To a solution of 3-(2-bromo-6-(trifluoromethyl)benzyl)oxetane-3-carboxylic acid (220 mg, 648 mol, 1.00 eq), and triethylamine (72.2 mg, 713 mol, 99.3 μL, 1.10 eq) in toluene (4.00 mL) was added diphenyl phosphoryl azide (178 mg, 648 mol, 140 L, 1.00 eq) dropwise at 0° C. The mixture was stirred at 80° C. for 1 h, then 2-methylpropan-2-ol (6.00 mL) was added to the mixture. The mixture was stirred at 80° C. for 4 h. The mixture was filtered and concentrated in vacuum. The mixture was purified by Prep-TLC (Petroleum ether/Ethyl acetate=5/1) to get tert-butyl (3-(2-bromo-6-(trifluoromethyl)benzyl)oxetan-3-yl)carbamate (160 mg, 390 mol, 60% yield) as a white solid. ¹H NMR (400 MHz, DMSO-d₆) δ=8.02 (br d, J=8.0 Hz, 1H), 7.84 (br s, 1H), 7.80 (br d, J=8.0 Hz, 1H), 7.45 (br t, J=8.0 Hz, 1H), 4.44-4.41 (m, 2H), 4.37-4.35 (m, 2H), 3.45 (s, 2H), 1.37 (s, 9H).

Step 5. To a solution of tert-butyl (3-(2-bromo-6-fluorobenzyl)oxetan-3-yl)carbamate (160 mg, 390 mol, 1.00 eq), (2-(2-diphenylphosphanylphenoxy)phenyl)-diphenyl-phosphane (42.0 mg, 78.0 mol, 0.200 eq), cesium carbonate (254 mg, 780 mol, 2.00 eq) in toluene (4.00 mL) was added palladium acetate (8.76 mg, 39.0 mol, 0.100 eq) under nitrogen atmosphere. The mixture was stirred at 100° C. for 2 h. The reaction mixture was concentrated in vacuum to give a residue. The residue was purified by Prep-TLC (SiO₂, Petroleum ether/Ethyl acetate=5/1) to get tert-butyl 4-(trifluoromethyl)spiro[indoline-2,3′-oxetane]-1-carboxylate (110 mg, 334 mol, 86% yield) as a white solid. ¹H NMR (400 MHz, DMSO-d₆) δ=7.98 (br d, J=7.6 Hz, 1H), 7.41-7.35 (m, 1H), 7.27 (d, J=8.0 Hz, 1H), 5.28 (d, J=6.4 Hz, 2H), 4.57 (d, J=6.4 Hz, 2H), 3.81 (s, 2H), 1.60 (s, 9H).

Step 6. To a solution of tert-butyl 4-(trifluoromethyl)spiro[indoline-2,3′-oxetane]-1-carboxylate (50.0 mg, 152 mol, 1.00 eq) in dichloromethane (2.00 mL) and trifluoroacetic acid (0.200 mL) was stirred at 25° C. for 1 h. The reaction mixture was concentrated to give 4-(trifluoromethyl)spiro[indoline-2,3′-oxetane](50 mg, crude) as yellow oil. MS (ESI) m/z 229.9 [M+H]⁺

Step 7. To a solution of 3-(5-amino-1-oxo-isoindolin-2-yl)piperidine-2,6-dione (500 mg, 1.93 mmol, 1.00 eq), pyridine (457 mg, 5.79 mmol, 467 μL, 3.00 eq) in N,N-dimethyl formamide (5.00 mL) was added 2,2,2-trichloroethyl carbonochloridate (408 mg, 1.93 mmol, 258 μL, 1.00 eq) at 0° C. The mixture was stirred at 25° C. for 2 h. The mixture was diluted with water (30.0 ml) and filtered. The filter cake was triturated with ethanol (4.00 mL) at 20° C. for 15 min and filtered to give 2,2,2-trichloroethyl (2-(2,6-dioxopiperidin-3-yl)-1-oxoisoindolin-5-yl)carbamate (500 mg, 1.15 mmol, 60% yield) as a gray solid. ¹H NMR (400 MHz, DMSO-d₆) δ=10.99 (s, 1H), 10.57 (br s, 1H), 7.83 (s, 1H), 7.68 (d, J=8.4 Hz, 1H), 7.58 (br d, J=8.0 Hz, 1H), 5.09 (dd, J=5.2, 13.2 Hz, 1H), 4.98 (s, 2H), 4.49-4.39 (m, 1H), 4.33-4.25 (m, 1H), 2.97-2.89 (m, 1H), 2.59 (br d, J=16.4 Hz, 1H), 2.40-2.35 (m, 1H), 2.03-1.94 (m, 1H). MS (ESI) m/z 436.1 [M+H]⁺

Step 8. To a solution of 4-(trifluoromethyl)spiro[indoline-2,3′-oxetane](5 mg, 14.57 mol, 1.00 eq, trifluoroacetic acid) in N,N-dimethyl formamide (0.500 mL) was added sodium hydride (874 g, 21.8 mol, 60% purity, 1.50 eq) at 0° C. After 0.5 h, 2,2,2-trichloroethyl (2-(2,6-dioxopiperidin-3-yl)-1-oxoisoindolin-5-yl)carbamate (12.7 mg, 29.1 mol, 2.00 eq) was added to the mixture. The mixture was stirred at 25° C. for 12 h. The mixture was adjusted to pH=5-6 with formic acid and filtered. The filtrate was diluted with water (10.0 mL), extracted with ethyl acetate (3×10.0 mL). The combined organic phase was washed with brine (10.0 mL), dried over anhydrous sodium sulfate, filtered and concentrated in vacuum to give a residue. The residue was purified by Prep-TLC (SiO₂, Petroleum ether/Ethyl acetate=0/1) and purified by reversed phase HPLC (column: spherical C18, 20-45 um, 100 Å, SW 40, mobile phase: [water (0.1% formic acid)-acetonitrile]) and lyophilized to get a crude product. The crude product was purified by Prep-HPLC (column: Welch ultimate C18 150*25 mm*7 um; mobile phase: [water water (formic acid)-acetonitrile]; gradient: 32%-62% B over 10 min) and lyophilized to afford N-(2-(2,6-dioxopiperidin-3-yl)-1-oxoisoindolin-5-yl)-4-(trifluoromethyl)spiro[indoline-2,3′-oxetane]-1-carboxamide (1.22 mg, 1.98 mol, 10% yield, 95% purity) as a white solid. ¹H NMR (400 MHz, DMSO-d₆) δ=10.98 (br s, 1H), 10.04 (s, 1H), 7.89 (s, 1H), 7.69 (d, J=8.4 Hz, 1H), 7.62-7.55 (m, 2H), 7.41 (t, J=8.0 Hz, 1H), 7.26 (d, J=8.0 Hz, 1H), 5.32 (d, J=6.4 Hz, 2H), 5.10 (dd, J=5.2, 13.2 Hz, 1H), 4.72 (d, J=6.4 Hz, 2H), 4.50-4.40 (m, 1H), 4.37-4.27 (m, 1H), 3.73 (s, 2H), 2.98-2.85 (m, 1H), 2.64-2.59 (m, 1H), 2.40 (br dd, J=4.4, 12.8 Hz, 1H), 2.05-1.95 (m, 1H). MS (ESI) m/z 515.1 [M+H]⁺

### Example 73. Synthesis of Compound 313

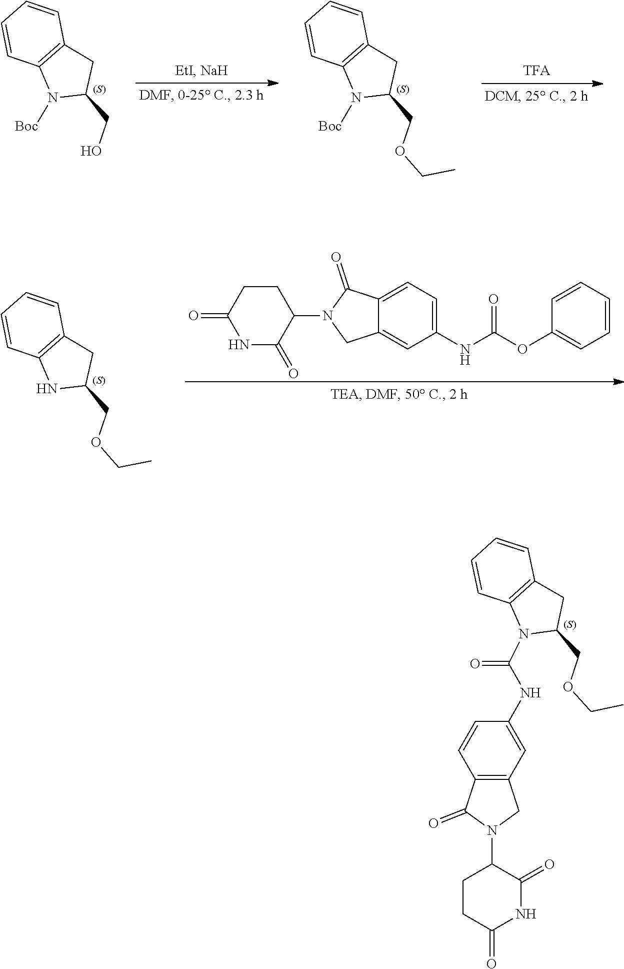

Step 1. To a mixture of tert-butyl (2S)-2-(hydroxymethyl)indoline-1-carboxylate (300 mg, 1.20 mmol, 1.00 eq) in dimethylformamide (3.00 mL) was added sodium hydride (72.1 mg, 1.81 mmol, 60% purity, 1.50 eq) at 0° C. The mixture was stirred at 0° C. for 20 min. Then the mixture was added ethyl iodide (281 mg, 1.81 mmol, 144 μL, 1.50 eq) and stirred at 25° C. for 2 h. The reaction mixture was quenched by addition saturated ammonium chloride aqueous solution (30 mL), then the mixture was stirred at 0° C. for 0.5 h. The diluted reaction mixture was extracted with ethyl acetate (3×30 mL), washed with brine (30 mL), dried over anhydrous sodium sulfate, filtered via vacuum filtration, and concentrated in vacuum. The residue was purified by column chromatography (SiO₂, Petroleum ether/Ethyl acetate=1/0 to 1/1) and concentrated in vacuum to give tert-butyl (S)-2-(ethoxymethyl)indoline-1-carboxylate (120 mg, 432 mol, 35% yield) as yellow oil. ¹H NMR (400 MHz, DMSO-d₆) δ=7.55 (br d, J=6.6 Hz, 1H), 7.19 (d, J=7.4 Hz, 1H), 7.12 (t, J=7.7 Hz, 1H), 6.98-6.86 (m, 1H), 4.48 (ddd, J=3.4, 6.8, 10.1 Hz, 1H), 3.61-3.33 (m, 4H), 3.25 (br dd, J=9.9, 16.4 Hz, 1H), 2.90 (dd, J=2.1, 16.5 Hz, 1H), 1.51 (s, 9H), 1.07 (t, J=6.9 Hz, 3H).

Step 2. To a mixture of tert-butyl (S)-2-(ethoxymethyl)indoline-1-carboxylate (120 mg, 432 mol, 1.00 eq) in dichloromethane (5.00 mL) was added trifluoroacetic acid (1.54 g, 13.4 mmol, 1.00 mL, 31.1 eq). The mixture was stirred at 25° C. for 2 h. The reaction was concentrated under reduced pressure to give (S)-2-(ethoxymethyl)indoline (120 mg, crude) as orange oil. MS (ESI) m/z 178.4 [M+H]⁺

Step 3. To a mixture of (S)-2-(ethoxymethyl)indoline (120 mg, 677 mol, 1.00 eq) in dimethylformamide (1.50 mL) was added phenyl (2-(2,6-dioxopiperidin-3-yl)-1-oxoisoindolin-5-yl)carbamate (256 mg, 677 mol, 1.00 eq) and triethylamine (205 mg, 2.03 mmol, 282 μL, 3.00 eq). The mixture was stirred at 50° C. for 2 h. The reaction was concentrated under reduced pressure to give a residue. The crude product was purified by reversed phase (column: spherical C18, 20-45 um, 100 Å, SW 80, mobile phase: [water (0.1% formic acid)-ACN) and lyophilized to give (2S)—N-(2-(2,6-dioxopiperidin-3-yl)-1-oxoisoindolin-5-yl)-2-(ethoxymethyl)indoline-1-carboxamide (54.3 mg, 116 μmol, 17% yield, 99% purity) as a off-white solid. ¹H NMR (400 MHz, DMSO-d₆) δ=10.98 (s, 1H), 9.19 (s, 1H), 7.82 (s, 1H), 7.75 (d, J=8.1 Hz, 1H), 7.64 (s, 1H), 7.51 (br d, J=7.6 Hz, 1H), 7.22 (br d, J=7.4 Hz, 1H), 7.15 (t, J=7.7 Hz, 1H), 7.00-6.89 (m, 1H), 5.09 (dd, J=5.0, 13.3 Hz, 1H), 4.92 (q, J=6.7 Hz, 1H), 4.49-4.39 (m, 1H), 4.34-4.24 (m, 1H), 3.64-3.44 (m, 4H), 3.37 (br s, 1H), 2.99-2.86 (m, 1H), 2.80 (br d, J=16.4 Hz, 1H), 2.69-2.55 (m, 1H), 2.38 (dq, J=4.3, 13.2 Hz, 1H), 2.04-1.94 (m, 1H), 1.13 (t, J=6.9 Hz, 3H). MS (ESI) m/z 463.4 [M+H]⁺

### Example 74. Synthesis of Compound 316

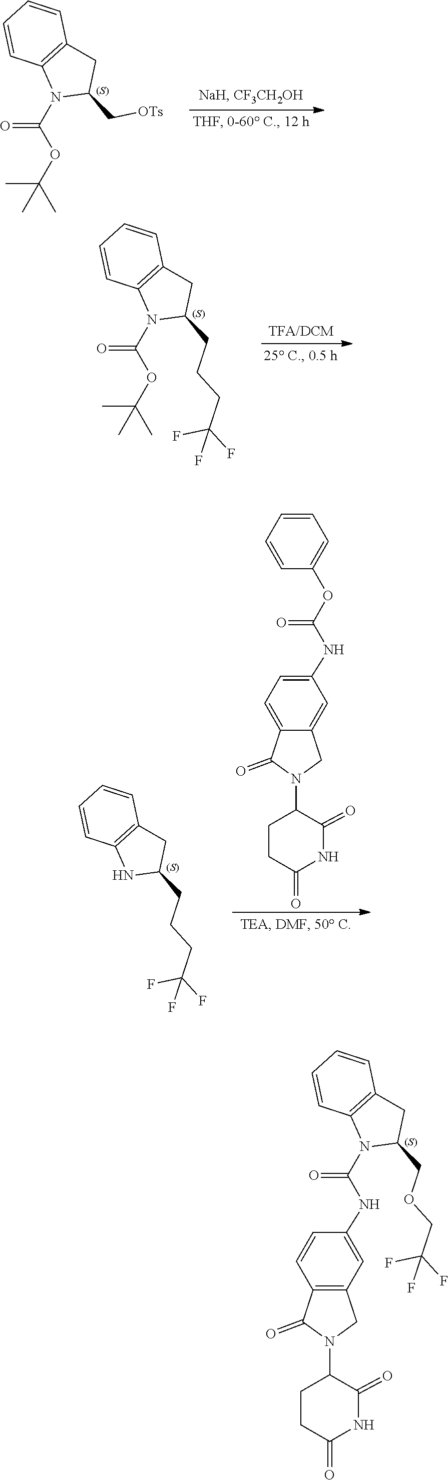

Step 1. To a solution of 2,2,2-trifluoroethan-1-ol (99.2 mg, 991 mol, 71.3 μL, 2.00 eq) in tetrahydrofuran (5.00 mL) was added sodium hydride (79.3 mg, 1.98 mmol, 60% purity, 4.00 eq) at 0° C. under nitrogen atmosphere. The mixture was stirred at 0° C. for 0.5 h, then tert-butyl (S)-2-((tosyloxy) methyl) indoline-1-carboxylate (200 mg, 496 mol, 1.00 eq) was added. The resulting mixture was stirred 60° C. for 11.5 h. The reaction mixture was quenched by saturated ammonium chloride (10 mL), then extracted with ethyl acetate (3×10 mL). The combined organic layers were washed with brine (2×10 mL), dried over sodium sulfate, filtered and concentrated under reduced pressure to afford a residue. The residue was purified by column chromatography (SiO₂, Petroleum ether/Ethyl acetate=10/1 to 0/1) and concentrated to afford tert-butyl (S)-2-((2, 2,2-trifluoroethoxy) methyl) indoline-1-carboxylate (60.0 mg, 181 mol, 36% yield) as yellow oil. ¹H NMR (400 MHz, DMSO-d₆) δ=7.75-7.37 (m, 1H), 7.20 (d, J=7.4 Hz, 1H), 7.13 (t, J=7.7 Hz, 1H), 6.98-6.88 (m, 1H), 4.52 (tdd, J=3.3, 6.7, 10.1 Hz, 1H), 4.23-3.96 (m, 2H), 3.75 (dd, J=3.9, 9.4 Hz, 1H), 3.60 (dd, J=7.1, 9.3 Hz, 1H), 2.89 (dd, J=2.3, 16.4 Hz, 1H), 1.51 (s, 9H).

Step 2. To a solution of tert-butyl (S)-2-((2, 2,2-trifluoroethoxy) methyl) indoline-1-carboxylate (60.0 mg, 181 mol, 1.00 eq) in dichloromethane (1.00 mL) was added trifluoroacetic acid (1.54 g, 13.5 mmol, 1.00 mL, 74.3 eq). The mixture was stirred at 25° C. for 0.5 h. The reaction was concentrated to afford (S)-2-((2,2,2-trifluoroethoxy)methyl)indoline (40.0 mg, 173 mol, 95% yield) was obtained as yellow oil. MS (ESI) m/z 232.2 [M+H]⁺

Step 3. To a solution of (S)-2-((2,2,2-trifluoroethoxy)methyl) indoline (40.0 mg, 173.00 mol, 1.00 eq) in dimethyl formamide (1.00 mL) was added triethylamine (52.5 mg, 519 mol, 72.2 μL, 3.00 eq) and phenyl (2-(2,6-dioxopiperidin-3-yl)-1-oxoisoindolin-5-yl)carbamate (65.6 mg, 173 mol, 1.00 eq). The reaction was stirred at 50° C. for 12 h. The reaction mixture was filtered. The filtrate was purified by Prep-HPLC (column: Phenomenex luna C18 150*25 mm*10 um; mobile phase: [water (acetic acid)-acetonitrile]; gradient:38%-68% B over 9 min) and lyophilized to afford (2S)—N-(2-(2,6-dioxopiperidin-3-yl)-1-oxoisoindolin-5-yl)-2-((2,2,2-trifluoroethoxy)methyl)indoline-1-carboxamide (31.6 mg, 58.8 mol, 34% yield, 96% purity) as a blue solid. ¹H NMR (400 MHz, DMSO-d₆) δ=10.98 (br s, 1H), 9.00 (d, J=1.0 Hz, 1H), 7.85 (s, 1H), 7.80 (d, J=8.2 Hz, 1H), 7.68-7.64 (m, 1H), 7.61-7.55 (m, 1H), 7.25 (d, J=7.2 Hz, 1H), 7.16 (t, J=7.6 Hz, 1H), 7.01-6.93 (m, 1H), 5.10 (dd, J=5.2, 13.2 Hz, 1H), 5.07-4.93 (m, 1H), 4.50-4.40 (m, 1H), 4.36-4.26 (m, 1H), 4.14 (q, J=9.2 Hz, 2H), 3.80-3.73 (m, 1H), 3.70-3.63 (m, 1H), 3.42-3.38 (m, 1H), 2.95-2.91 (m, 1H), 2.91-2.85 (m, 1H), 2.64-2.57 (m, 1H), 2.47-2.36 (m, 1H), 2.07-1.93 (m, 1H). MS (ESI) m/z 517.2 [M+H]⁺

### Example 75. Synthesis of Compound 309

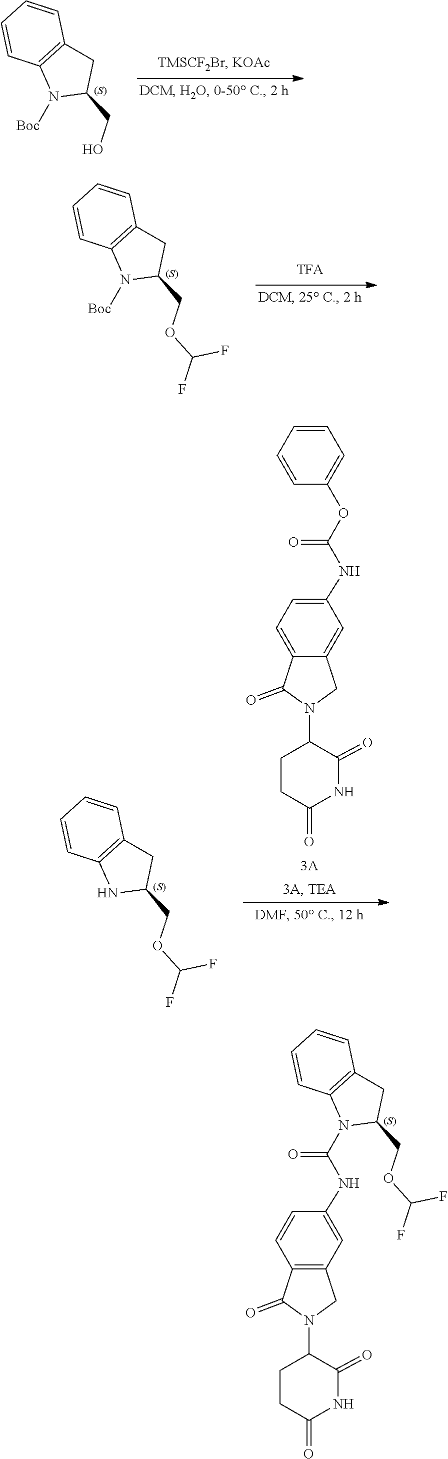

Step 1. To a solution of tert-butyl (S)-2-(hydroxymethyl)indoline-1-carboxylate (500 mg, 2.01 mmol, 1.00 eq) in dichloromethane (5.00 mL) and water (5.00 mL) was added potassium acetate (1.18 g, 12.0 mmol, 6.00 eq) and (bromodifluoromethyl)trimethylsilane (1.30 g, 6.42 mmol, 3.20 eq) at 0° C., the mixture was stirred at 50° C. for 2 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by column chromatography (SiO₂, Petroleum ether/Ethyl acetate=20/1 to 5/1) to afford tert-butyl (S)-2-((difluoromethoxy) methyl) indoline-1-carboxylate (300 mg, 1.00 mmol, 50% yield) as a yellow solid. ¹H NMR (400 MHz, DMSO-d₆) δ=7.55 (br d, J=1.2 Hz, 1H), 7.17 (d, J=7.4 Hz, 1H), 7.11 (t, J=7.6 Hz, 1H), 6.91 (t, J=7.2 Hz, 1H), 6.64 (t, J=75.4 Hz, 1H), 4.60-4.50 (m, 1H), 3.94 (d, J=4.6 Hz, 2H), 3.37-3.29 (m, 1H), 2.88 (dd, J=2.8, 16.6 Hz, 1H), 1.49 (s, 9H).

Step 2. A solution of tert-butyl (S)-2-((difluoromethoxy)methyl)indoline-1-carboxylate (100 mg, 334 mol, 1.00 eq) in trifluoroacetic acid (0.200 mL) and dichloromethane (1.00 mL), the mixture was stirred at 25° C. for 2 h. The reaction mixture was concentrated under reduced pressure to afford (S)-2-((difluoromethoxy)methyl) indoline (60.0 mg, 301 mol, 90% yield) as yellow oil. MS (ESI) m/z 200.1 [M+H]⁺

Step 3. To a solution of (S)-2-((difluoromethoxy)methyl)indoline (60.0 mg, 301 mol, 1.00 eq) and phenyl (2-(2,6-dioxopiperidin-3-yl)-1-oxoisoindolin-5-yl)carbamate (137 mg, 361 mol, 1.20 eq) in dimethylformamide (1.00 mL) was added triethylamine (91.4 mg, 904 mol, 126 μL, 3.00 eq), the mixture was stirred at 50° C. for 12 h. The mixture was filtered to give filtrate. The filtrate was purified by Prep-HPLC (column: Phenomenex luna C18 150*25 mm*10 um; mobile phase: [water (FA)-ACN]; gradient:30%-60% B over 10 min) to afford (2S)-2-((difluoromethoxy)methyl)-N-(2-(2,6-dioxopiperidin-3-yl)-1-oxoisoindolin-5-yl)indoline-1-carboxamide (43.11 mg, 88.1 mol, 29% yield, 99% purity) as a gray solid.

¹H NMR (400 MHz, DMSO-d₆) δ=10.97 (s, 1H), 9.05 (s, 1H), 7.85 (s, 1H), 7.79 (d, J=8.0 Hz, 1H), 7.68-7.64 (m, 1H), 7.62-7.58 (m, 1H), 7.24 (d, J=7.6 Hz, 1H), 7.16 (t, J=7.6 Hz, 1H), 6.99-6.93 (m, 1H), 6.67 (t, J=75.6 Hz, 1H), 5.15-5.04 (m, 2H), 4.49-4.40 (m, 1H), 4.34-4.26 (m, 1H), 3.95 (d, J=5.0 Hz, 2H), 3.41 (br dd, J=9.8, 16.2 Hz, 1H), 3.00-2.87 (m, 2H), 2.60 (br d, J=18.0 Hz, 1H), 2.46-2.36 (m, 1H), 2.05-1.95 (m, 1H). MS (ESI) m/z 485.2 [M+H]⁺

### Example 76. Synthesis of Compound 312

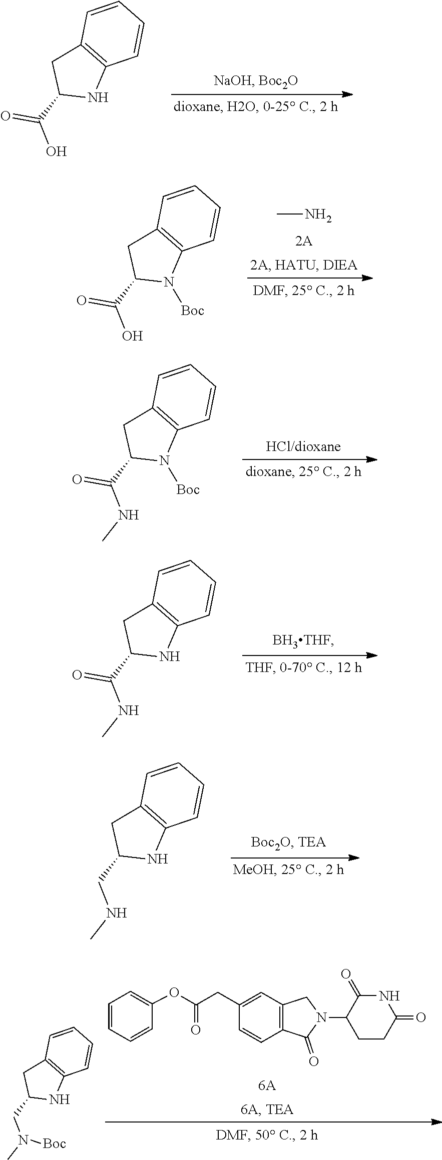

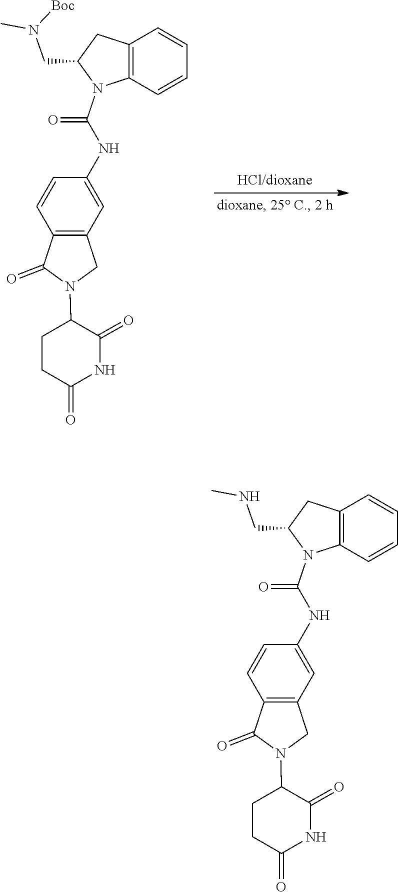 [SMILES: CN(C[C@@H]1Cc2ccccc2N1C(=O)Nc1ccc2c(c1)CN(C1CCC(=O)NC1=O)C2=O)C(=O)OC(C)(C)C ; CNC[C@@H]1Cc2ccccc2N1C(=O)Nc1ccc2c(c1)CN(C1CCC(=O)NC1=O)C2=O]

Step 1. To a solution of (S)-indoline-2-carboxylic acid (1.00 g, 6.13 mmol, 1.00 eq) in dioxane (5.00 mL) and water (5.00 mL) was added sodium hydroxide (294 mg, 7.35 mmol, 1.20 eq) and di-tert-butyl dicarbonate (2.01 g, 9.19 mmol, 2.11 mL, 1.50 eq) at 0° C., the mixture was stirred at 25° C. for 2 h. The reaction mixture was quenched by addition citric acid (10%, 100 mL) at 25° C., and then extracted with ethyl acetate (3×50 mL). The combined organic layers were washed with brine (3×20 mL), dried over anhydrous sodium sulfate, filtered and concentrated under reduced pressure to give (S)-1-(tert-butoxycarbonyl)indoline-2-carboxylic acid (2.00 g, crude) as yellow oil. ¹H NMR (400 MHz, DMSO-d₆) δ=12.91-12.71 (m, 1H), 7.78-7.72 (m, 1H), 7.21-7.14 (m, 2H), 6.97-6.91 (m, 1H), 4.80-4.74 (m, 1H), 3.55-3.47 (m, 1H), 3.08-2.98 (m, 1H), 1.47-1.44 (m, 9H). LC-MS (ESI) m/z 163.9 [M+H−100]⁺

Step 2. To a solution of (S)-1-(tert-butoxycarbonyl)indoline-2-carboxylic acid (2.00 g, 7.60 mmol, 1.00 eq) in dimethyl formamide (20.0 mL) was added methanamine; hydrochloride (5.13 g, 76.0 mmol, 10.0 eq), O-(7-azabenzotriazol-1-yl)-N,N,N,N-tetramethyluroniumhexafluorophosphate (4.33 g, 11.4 mmol, 1.50 eq) and diisopropylethylamine (11.8 g, 91.2 mmol, 15.9 mL, 12.0 eq), the mixture was stirred at 25° C. for 2 h. The reaction mixture was diluted with water (80 mL) and exacted with ethyl acetate (3×30 mL). The organic phase was separated, washed with brine (2×40 ml), dried over anhydrous sodium sulfate, filtered and concentrated under reduced pressure to give a residue. The residue was purified by column chromatography (SiO₂, petroleum ether/Ethyl acetate=10/1 to 0/1) to afford tert-butyl (S)-2-(methylcarbamoyl)indoline-1-carboxylate (2.40 g, crude) as a white solid. ¹H NMR (400 MHz, DMSO-d₆) δ=8.04-7.97 (m, 1H), 7.80-7.65 (m, 1H), 7.20-7.08 (m, 2H), 6.97-6.86 (m, 1H), 4.72-4.62 (m, 1H), 3.46-3.37 (m, 1H), 2.89-2.84 (m, 1H), 2.67-2.56 (m, 3H), 1.55-1.35 (m, 9H). LC-MS (ESI) m/z 177.0 [M+H−100]⁺

Step 3. To a solution of tert-butyl (S)-2-(methylcarbamoyl)indoline-1-carboxylate (1.40 g, 5.07 mmol, 1.00 eq) in dioxane (10.0 mL) was added hydrochloric acid/dioxane (4.00 M, 14.0 mL, 11.0 eq) at 25° C., the mixture was stirred at 25° C. for 2 h. The mixture was concentrated under reduced pressure to give a residue. The mixture was concentrated under reduced pressure to give a residue. The residue was triturated with ethyl acetate (5.00 ml) and filtered. The filter cake was washed with ethyl acetate and dried to afford (S)—N-methylindoline-2-carboxamide (1.00 g, crude) as a yellow solid. ¹H NMR (400 MHz, DMSO-d₆) δ=8.37-8.25 (m, 1H), 7.27-7.14 (m, 2H), 7.07-6.96 (m, 2H), 4.52-4.49 (m, 1H), 3.47-3.39 (m, 1H), 3.11-3.03 (m, 1H), 2.68-2.65 (m, 3H). LC-MS (ESI) m/z 177.1 [M+H]⁺

Step 4. To a solution of (S)—N-methylindoline-2-carboxamide (500 mg, 2.84 mmol, 1.00 eq) in tetrahydrofuran (5.00 mL) was added borane tetrahydrofuran (1.00 M, 28.4 mL, 10.0 eq) at 0° C., the mixture was stirred 25° C. for 30 min, then the mixture was stirred at 70° C. for 11.5 h. The mixture was quenches with methanol (10.0 mL) and added hydrochloric acid at 25° C., the mixture was stirred at 70° C. for 1 h. The mixture was concentrated under reduced pressure to give a residue. The residue was purified by reversed phase (neutral condition) to give (S)-1-(indolin-2-yl)-N-methylmethanamine (800 mg, crude) as yellow oil. LC-MS (ESI) m/z 163.0 [M+H]⁺

Step 5. To a solution of (S)-1-(indolin-2-yl)-N-methylmethanamine (360 mg, 2.22 mmol, 1.00 eq) in methanol (4.00 mL) was added di-tert-butyl dicarbonate (387 mg, 1.78 mmol, 408 μL, 0.800 eq) and triethylamine (449 mg, 4.44 mmol, 618 μL, 2.00 eq) at 25° C., the mixture was stirred at 25° C. for 2 h. The reaction mixture was diluted with water (30.0 mL) and exacted with ethyl acetate (3×30 mL). The organic phase was separated, washed with brine (2×10 mL), dried over anhydrous sodium sulfate, filtered and concentrated under reduced pressure to give a residue. The residue was purified by column chromatography (SiO₂, petroleum ether/Ethyl acetate=10/1) to afford tert-butyl (S)-(indolin-2-ylmethyl)(methyl)carbamate (120 mg, 457 μmol, 21% yield) as yellow oil. ¹H NMR (400 MHz, DMSO-d₆) δ=7.02-6.97 (m, 1H), 6.92-6.86 (m, 1H), 6.54-6.45 (m, 2H), 5.65-5.59 (m, 1H), 3.98-3.88 (m, 1H), 3.31-3.24 (m, 1H), 3.21-3.14 (m, 1H), 3.02-2.92 (m, 1H), 2.89-2.81 (m, 3H), 2.67-2.58 (m, 1H), 1.42-1.36 (m, 9H). LC-MS (ESI) m/z 163.1 [M+H−100]⁺

Step 6. To a solution of tert-butyl (S)-(indolin-2-ylmethyl)(methyl)carbamate (120 mg, 457 mol, 1.00 eq) in dimethyl formamide (3.00 mL) was added phenyl N-[2-(2,6-dioxo-3-piperidyl)-1-oxo-isoindolin-5-yl]carbamate (174 mg, 457 mol, 1.00 eq) and triethylamine (139 mg, 1.37 mmol, 191 μL, 3.00 eq) at 25° C., the mixture was stirred at 25° C. for 2 h. The mixture was filtered to give filtrate. The filtrate was purified by reversed phase (0.1% formic acid condition) to give tert-butyl (((2S)-1-((2-(2,6-dioxopiperidin-3-yl)-1-oxoisoindolin-5-yl)carbamoyl)indolin-2-yl)methyl)(methyl)carbamate (125 mg, 228 mol, 50% yield) as yellow oil. ¹H NMR (400 MHz, DMSO-d₆) δ=11.00-10.97 (m, 1H), 9.33-9.17 (m, 1H), 7.95-7.80 (m, 2H), 7.66-7.63 (m, 1H), 7.30 (br d, J=8.3 Hz, 2H), 7.21-7.14 (m, 1H), 7.00-6.94 (m, 1H), 5.11-5.07 (m, 1H), 4.96-4.82 (m, 1H), 4.46-4.41 (m, 1H), 4.32-4.28 (m, 1H), 3.46-3.44 (m, 1H), 2.93-2.84 (m, 4H), 2.78-2.75 (m, 1H), 2.66-2.56 (m, 3H), 2.42-2.35 (m, 1H), 2.01 (br s, 1H), 1.44-1.32 (m, 9H). LC-MS (ESI) m/z 448.2 [M+H−100]⁺

Step 7. To a solution of tert-butyl (((2S)-1-((2-(2,6-dioxopiperidin-3-yl)-1-oxoisoindolin-5-yl)carbamoyl)indolin-2-yl)methyl)(methyl)carbamate (120 mg, 219 mol, 1.00 eq) in dioxane (2.00 mL) was added hydrochloric acid/dioxane (4.00 M, 2.00 mL, 36.5 eq) at 25° C., the mixture was stirred at 30° C. for 2 h. The reaction mixture was concentrated under reduced pressure to give a residue. The residue was purified by reversed phase (0.1% hydrochloric acid condition) to give (2S)—N-(2-(2,6-dioxopiperidin-3-yl)-1-oxoisoindolin-5-yl)-2-((methylamino)methyl)indoline-1-carboxamide (32.41 mg, 73.8 mol, 34% yield) as a white solid. ¹H NMR (400 MHz, DMSO-d₆) δ=11.02-10.95 (m, 1H), 9.34-9.26 (m, 1H), 8.94-8.73 (m, 2H), 7.96-7.87 (m, 1H), 7.82-7.73 (m, 1H), 7.70-7.57 (m, 2H), 7.34-7.27 (m, 1H), 7.25 (br s, 1H), 7.06-7.00 (m, 1H), 5.13-5.01 (m, 2H), 4.48-4.40 (m, 1H), 4.33-4.26 (m, 1H), 3.46-3.39 (m, 1H), 3.12-2.89 (m, 4H), 2.66-2.62 (m, 1H), 2.62-2.58 (m, 3H), 2.42-2.35 (m, 1H), 2.05-1.97 (m, 1H). LC-MS (ESI) m/z 448.1 [M+H]⁺

### Example 77. Synthesis of Compound 297

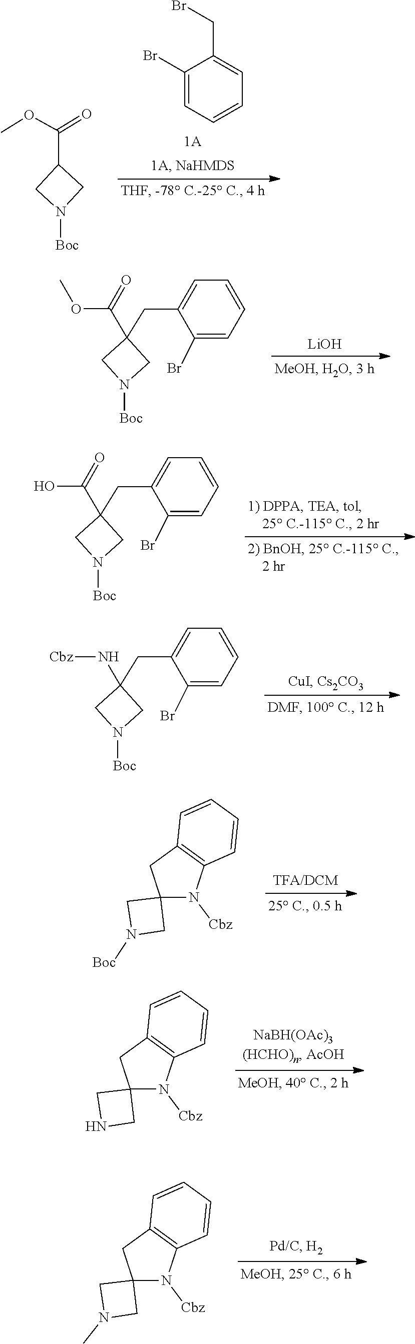

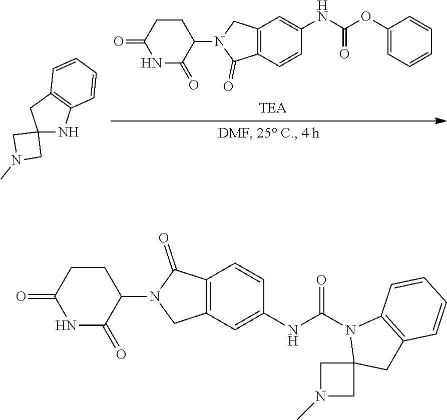 [SMILES: CN1CC2(Cc3ccccc3N2)C1 ; O=C1CCC(N2Cc3cc(NC(=O)Oc4ccccc4)ccc3C2=O)C(=O)N1 ; CN1CC2(Cc3ccccc3N2C(=O)Nc2ccc3c(c2)CN(C2CCC(=O)NC2=O)C3=O)C1]

Step 1. To a solution of 1-(tert-butyl) 3-methyl azetidine-1,3-dicarboxylate (10.0 g, 46.4 mmol, 1.00 eq) and 1-bromo-2-(bromomethyl)benzene (13.9 g, 55.7 mmol, 1.20 eq) in tetrahydrofuran (100 mL) was added dropwise double (trimethylsilyl) sodium amino (1.00 M, 55.7 mL, 1.20 eq) at −78° C. over 30 min. After addition, the mixture was stirred at 25° C. for 3.5 hours. The mixture was added saturated ammonium chloride (100 mL) and extracted with ethyl acetate (3×100 mL). The combined organic layers were dried over sodium sulfate and concentrated. The residue was purified by column chromatography (silicon dioxide, petroleum ether/ethyl acetate=100/1 to 5/1) to give 1-(tert-butyl) 3-methyl 3-(2-bromobenzyl)azetidine-1,3-dicarboxylate (12.0 g, 31.3 mmol, 67% yield) as yellow oil. ¹H NMR (400 MHz, methanol-d₄) δ=7.56 (d, J=7.6 Hz, 1H), 7.28-7.21 (m, 1H), 7.15-7.08 (m, 2H), 4.14 (br d, J=8.8 Hz, 2H), 3.92 (d, J=8.8 Hz, 2H), 3.69 (s, 3H), 3.36 (s, 2H), 1.37 (s, 9H).

Step 2. To a solution of 1-(tert-butyl) 3-methyl 3-(2-bromobenzyl)azetidine-1,3-dicarboxylate (12.0 g, 31.2 mmol, 1.00 eq) in methanol (100 mL) was added lithium hydroxide (3.74 g, 156 mmol, 156 mL, 5.00 eq) in water (50.0 mL). The mixture was stirred at 25° C. for 3 hours. The methanol was concentrated in vacuum. The residue was added ethyl acetate (200 mL) and acidized with aqueous chlorhydric acid (20.0 mL). The aqueous phase was extracted with ethyl acetate (3×150 mL). The combined organic layers were dried over sodium sulfate and concentrated in vacuum. The residue was purified by column chromatography (silicon dioxide, petroleum ether/ethyl acetate=50/1 to 2/1) to give 3-(2-bromobenzyl)-1-(tert-butoxycarbonyl)azetidine-3-carboxylic acid (9.40 g, 24.6 mmol, 78% yield, 97% purity) as a yellow solid. ¹H NMR (400 MHz, DMSO-d₆) δ=13.03 (s, 1H), 7.64 (dd, J=1.2, 8.0 Hz, 1H), 7.37-7.30 (m, 1H), 7.22-7.11 (m, 2H), 4.12-4.04 (m, 2H), 3.85 (br d, J=8.4 Hz, 2H), 3.30 (s, 2H), 1.34 (s, 9H).

Step 3. To a solution of 3-(2-bromobenzyl)-1-(tert-butoxycarbonyl)azetidine-3-carboxylic acid (9.00 g, 24.3 mmol, 1.00 eq) in toluene (30.0 mL) was added dropwise diphenyl phosphoryl azide (10.0 g, 36.4 mmol, 7.87 mL, 1.50 eq) and triethylamine (4.92 g, 48.6 mmol, 6.77 mL, 2.00 eq) at 25° C. After addition, the mixture was stirred at 115° C. for 2 hours, and then benzyl alcohol (5.26 g, 48.6 mmol, 5.04 mL, 2.00 eq) was added dropwise at 25° C. The resulting mixture was stirred at 115° C. for 2 hours. The solvent was concentrated in vacuum. The residue was added ethyl acetate (50.0 mL) and water (30.0 mL), the aqueous phase was extracted with ethyl acetate (3×30.0 mL). The combined organic layers were dried over sodium sulfate and concentrated in vacuum. The residue was purified by column chromatography (silicon dioxide, petroleum ether/ethyl acetate=100/1 to 10/1) to give tert-butyl 3-(((benzyloxy)carbonyl)amino)-3-(2-bromobenzyl)azetidine-1-carboxylate (9.42 g, 19.8 mmol, 81% yield) as yellow oil. MS (ESI) m/z. 429.1 [M+H−56]⁺

Step 4. To a solution of tert-butyl 3-(((benzyloxy)carbonyl)amino)-3-(2-bromobenzyl)azetidine-1-carboxylate (9.40 g, 19.7 mmol, 1.00 eq) in dimethyl formamide (50.0 mL) was added cesium carbonate (12.8 g, 39.5 mmol, 2.00 eq) and cuprous iodide (376 mg, 1.98 mmol, 0.100 eq) was degassed and purged with nitrogen for 3 times. The mixture was stirred at 100° C. for 12 hours under nitrogen atmosphere. The mixture was added water (50.0 mL) and extracted with ethyl acetate (3×100.0 mL). The combined organic layers were dried over sodium sulfate and concentrated. The residue was purified by column chromatography (silicon dioxide, petroleum ether/ethyl acetate=100/1 to 10/1) to give 1′-benzyl 1-(tert-butyl) spiro[azetidine-3,2′-indoline]-1,1′-dicarboxylate (6.70 g, 16.1 mmol, 81% yield, 95% purity) as yellow oil. ¹H NMR (400 MHz, DMSO-d₆) δ=7.78-7.54 (m, 1H), 7.49-7.44 (m, 2H), 7.43-7.32 (m, 3H), 7.22-7.11 (m, 2H), 7.00-6.94 (m, 1H), 5.33 (br s, 2H), 4.69-4.48 (m, 2H), 3.98-3.79 (m, 2H), 3.59 (s, 2H), 1.37 (s, 9H).

Step 5. To a solution of 1′-benzyl 1-(tert-butyl) spiro[azetidine-3,2′-indoline]-1,1′-dicarboxylate (3.00 g, 7.61 mmol, 1.00 eq) in dichloromethane (20.0 mL) was added trifluoroacetic acid (9.21 g, 80.7 mmol, 6.00 mL, 10.0 eq). The mixture was stirred at 25° C. for 0.5 hour. The solvent was concentrated in vacuum. The residue was added ethyl acetate (2.00 mL) and basified with saturated aqueous sodium bicarbonate (15.0 mL). The aqueous phase was extracted with ethyl acetate (4×2.00 mL). The combined organic layers were dried over sodium sulfate and concentrated in vacuum to give the crude product benzyl spiro[azetidine-3,2′-indoline]-1′-carboxylate (1.98 g, crude) as a yellow solid. ¹H NMR (400 MHz, DMSO-d₆) δ=7.53 (br d, J=7.2 Hz, 3H), 7.46-7.36 (m, 3H), 7.22 (d, J=7.6 Hz, 1H), 7.15 (t, J=8.0 Hz, 1H), 7.02-6.95 (m, 1H), 5.43 (s, 2H), 4.74 (br d, J=11.2 Hz, 2H), 4.11 (d, J=11.6 Hz, 2H), 3.64 (s, 2H).

Step 6. To a solution of benzyl spiro[azetidine-3,2′-indoline]-1′-carboxylate (1.80 g, 6.12 mmol, 1.00 eq) in methanol (20.0 mL) was added sodium carbonate (648 mg, 6.12 mmol, 1.00 eq) at 40° C. After addition, the mixture was stirred at 40° C. for 0.5 hour, and then paraformaldehyde (2.00 g, 30.5 mmol, 5.00 eq) was added at 40° C. The mixture was stirred at 40° C. for 0.5 hour, and then sodium cyanoborohydride (1.54 g, 24.4 mmol, 4.00 eq) was added at 40° C. The resulting mixture was stirred at 40° C. for 1 hour. The solvent was concentrated in vacuum. The residue was added ethyl acetate (10.0 mL) and water (10.0 mL). The aqueous phase was extracted with ethyl acetate (4×5 mL). The combined organic layers were dried over sodium sulfate and concentrated in vacuum. The residue was purified by column chromatography (silicon dioxide, dichloromethane:methanol=1:0 to 10:1) to give benzyl 1-methylspiro[azetidine-3,2′-indoline]-1′-carboxylate (1.50 g, 4.86 mmol, 79% yield) as a white solid. ¹H NMR (400 MHz, CDCl₃) δ=7.76-7.54 (m, 1H), 7.52-7.32 (m, 5H), 7.22-7.10 (m, 2H), 7.05-6.96 (m, 1H), 5.39 (s, 2H), 4.72-4.35 (m, 2H), 4.08-3.82 (m, 2H), 3.69 (s, 2H), 2.99-2.53 (m, 3H).

Step 7. To a mixture of benzyl 1-methylspiro[azetidine-3,2′-indoline]-1′-carboxylate (1.50 g, 4.86 mmol, 1.00 eq), palladium/carbon (1.04 g, 486 mol, 5.00% purity, 0.100 eq) in methanol (10.0 mL) was degassed and purged with hydrogen for 3 times, and then the mixture was stirred at 25° C. for 6 hours under hydrogen atmosphere. After the reaction was completed, the reaction liquid was filtered through the diatom soil layer under a mild nitrogen atmosphere. The solvent was concentrated in vacuum to give 1-methylspiro[azetidine-3,2′-indoline](514 mg, 2.95 mmol, 60% yield) as a gray solid. ¹H NMR (400 MHz, DMSO-d₆) δ=6.98 (d, J=7.2 Hz, 1H), 6.89 (t, J=7.6 Hz, 1H), 6.53-6.47 (m, 1H), 6.42 (d, J=7.6 Hz, 1H), 6.15 (s, 1H), 3.46 (br d, J=7.2 Hz, 2H), 3.16 (s, 2H), 3.13 (br d, J=7.6 Hz, 2H), 2.32 (s, 3H).

To a solution of 1-methylspiro[azetidine-3,2′-indoline](200 mg, 1.15 mmol, 1.00 eq) in dimethyl formamide (3.00 mL) was added triethylamine (232 mg, 2.30 mmol, 319 μL, 2.00 eq) and phenyl (2-(2,6-dioxopiperidin-3-yl)-1-oxoisoindolin-5-yl)carbamate (435 mg, 1.15 mmol, 1.00 eq). The mixture was stirred at 25° C. for 4 hours. The reaction solution was filtered to give filtrate. The crude product was purified by Prep-HPLC (column: Waters xbridge 150×25 mm 10 um; mobile phase: [water (ammonium bicarbonate)-acetonitrile]; gradient:22%-52% B over 18 min) to afford 5-cyclopropyl-N-(2-(2,6-dioxopiperidin-3-yl)-1-oxoisoindolin-5-yl)-1-(methoxymethyl)isoindoline-2-carboxamide (13.4 mg, 28.35 mol, 2% yield, FA) as a white solid. ¹H NMR (400 MHz, DMSO-d₆) 5=13.10 (s, 1H), 10.97 (br s, 1H), 8.23 (br s, 1H, FA), 8.04-7.91 (m, 2H), 7.67 (d, J=8.4 Hz, 1H), 7.50 (dd, J 1.6, 8.4 Hz, 1H), 7.19-7.08 (m, 2H). 6.91 (t, J=7.6 Hz, 1H), 5.10 (dd, J 5.2, 13.2 Hz, 1H), 4.50-4.39 (i, 1H), 4.36-4.25 (m, 1H), 4.01 (br d, J=10.0 Hz, 2H), 3.53 (br d, J 10.0 Hz, 2H), 3.40 (s, 2H), 2.98-2.84 (m, 1H), 2.69-2.57 (m, 1H), 2.55-2.52 (m, 3H), 2.39 (br dd, =4.8, 13.2 Hz, 1H), 2.04-1.95 (m, 1H). MS (ESI) m/z 460.0 [M+H]⁺

### Example 78. Synthesis of Compound 326

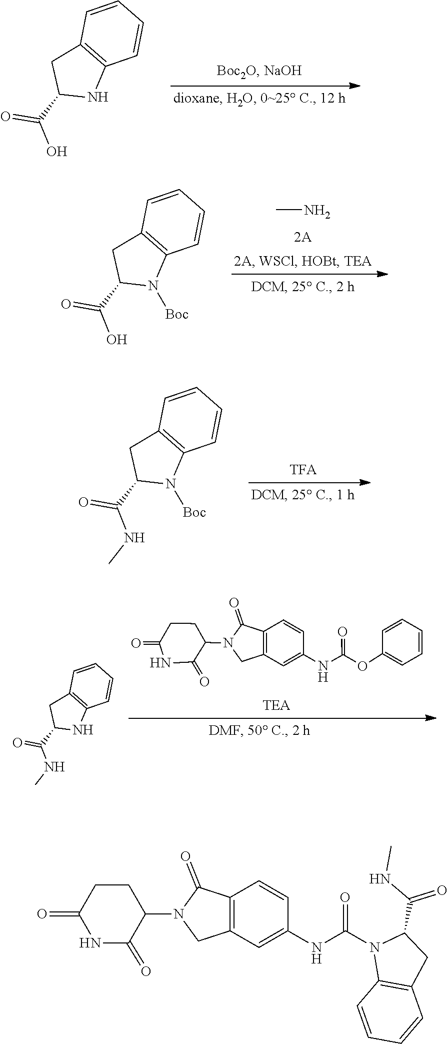

Step 1. To a solution of (S)-indoline-2-carboxylic acid (1.00 g, 6.13 mmol, 1.00 eq) in dioxane (5.00 mL) and water (5.00 mL) was added sodium hydroxide (294 mg, 7.35 mmol, 1.20 eq) and di-tert-butyldicarbonate (2.01 g, 9.19 mmol, 2.11 mL, 1.50 eq) at 0° C. Then the reaction mixture was stirred at 25° C. for 12 h. The reaction mixture was quenched by addition citric acid (10%, 100 mL) at 25° C., and then extracted with ethyl acetate (3×50 mL). The combined organic layers were washed with brine (3×50 mL), dried over sodium sulfate, filtered and concentrated under reduced pressure to give (S)-1-(tert-butoxycarbonyl)indoline-2-carboxylic acid (2.00 g, crude) as yellow oil. ¹H NMR (400 MHz, DMSO-d₆) δ=12.90 (br s, 1H), 7.74 (br d, J=7.6 Hz, 1H), 7.21-7.12 (m, 2H), 6.94 (t, J=7.4 Hz, 1H), 4.77 (dd, J=4.1, 11.6 Hz, 1H), 3.57-3.48 (m, 1H), 3.03 (br d, J=16.4 Hz, 1H), 1.48-1.43 (m, 9H).

Step 2. To a solution of (S)-1-(tert-butoxycarbonyl)indoline-2-carboxylic acid (200 mg, 759 mol, 1.00 eq) and methanamine (512 mg, 7.60 mmol, 10.0 eq, hydrochloric acid) in dichloromethane (1.00 mL) was added 3-(ethyliminomethyleneamino)-N,N-dimethyl-propan-1-amine; hydrochloride (291 mg, 1.52 mmol, 2.00 eq), 1H-benzo[d][1,2,3]triazol-1-ol (205 mg, 1.52 mmol, 2.00 eq) and triethylamine (768 mg, 7.60 mmol, 1.06 mL, 10.0 eq), the mixture was stirred at 25° C. for 2 h. The reaction mixture was poured into water (80.0 mL) and extracted with ethyl acetate (3×40 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=20/1 to 4/1) to afford-(S)-tert-butyl 2-(methylcarbamoyl)indoline-1-carboxylate (200 mg, 723 mol, 95% yield) as a yellow solid. ¹H NMR (400 MHz, DMSO-d₆) δ=8.11-7.90 (m, 1H), 7.79-7.63 (m, 1H), 7.20-7.06 (m, 2H), 6.97-6.86 (m, 1H), 4.72-4.56 (m, 1H), 3.47-3.38 (m, 1H), 2.93-2.83 (m, 1H), 2.65-2.53 (m, 3H), 1.52-1.31 (m, 9H).

Step 3. To a solution of (S)-tert-butyl 2-(methylcarbamoyl)indoline-1-carboxylate (80.0 mg, 289 mol, 1.00 eq) in dichloromethane (2.00 mL) was added trifluoroacetic acid (614 mg, 5.38 mmol, 400 μL, 18.6 eq), the mixture was stirred at 25° C. for 1 h. The reaction mixture was concentrated to give (S)—N-methylindoline-2-carboxamide (50.0 mg, 283 mol, 98% yield) as yellow oil. MS (ESI) m/z 177.1 [M+H]⁺

Step 4. To a solution of (S)—N-methylindoline-2-carboxamide (50.0 mg, 283 mol, 1.00 eq) and triethylamine (114 mg, 1.13 mmol, 157 μL, 4.00 eq) in dimethylformamide (2.00 mL) was added phenyl (2-(2,6-dioxopiperidin-3-yl)-1-oxoisoindolin-5-yl)carbamate (96.8 mg, 255 mol, 0.900 eq), the mixture was stirred at 50° C. for 2 h. The reaction mixture was filtered to give a filtrate. The filtrate was purified by Prep-HPLC (column: Phenomenex luna C18 150*25 mm*10 um; mobile phase: [water (FA)-ACN]; B %: 12%-42%, 10 min) to afford (2S)—N¹-(2-(2,6-dioxopiperidin-3-yl)-1-oxoisoindolin-5-yl)-N²-methylindoline-1,2-dicarboxamide (25.04 mg, 54.3 mol, 19% yield) as a white solid. ¹H NMR (400 MHz, DMSO-d₆) δ=10.97 (br s, 1H), 9.07 (s, 1H), 8.05 (q, J=4.0 Hz, 1H), 7.81 (s, 1H), 7.74 (d, J=8.0 Hz, 1H), 7.67-7.61 (m, 1H), 7.60-7.52 (m, 1H), 7.26-7.11 (m, 2H), 6.94 (t, J=7.4 Hz, 1H), 5.19-5.05 (m, 2H), 4.47-4.39 (m, 1H), 4.34-4.24 (m, 1H), 3.59-3.46 (m, 1H), 3.09 (dd, J=2.9, 16.4 Hz, 1H), 2.98-2.84 (m, 1H), 2.68-2.61 (m, 1H), 2.59 (d, J=4.5 Hz, 3H), 2.41 (br dd, J=9.0, 13.1 Hz, 1H), 2.07-1.97 (m, 1H). MS (ESI) m/z 462.0 [M+H]⁺

### Example 79. Synthesis of Compound 327

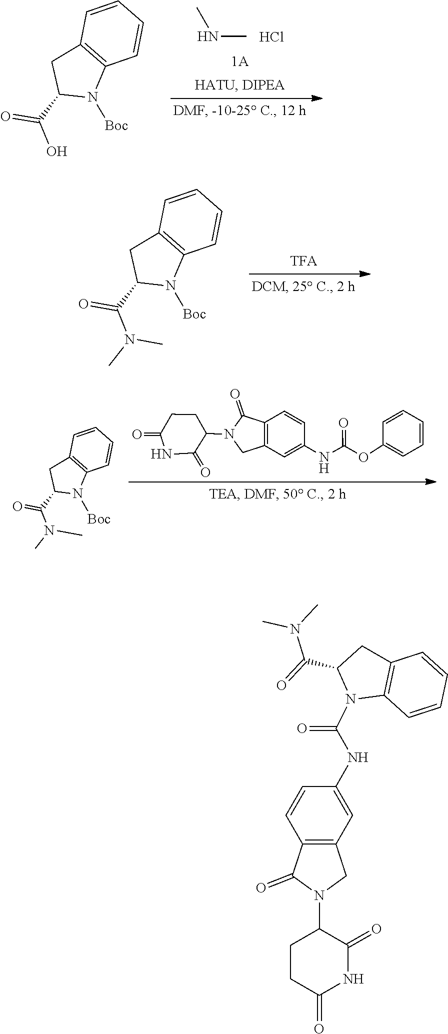

Step 1. A solution of (S)-1-(tert-butoxycarbonyl)indoline-2-carboxylic acid (200 mg, 760 mol, 1.00 eq), in dimethyl formamide (5.00 mL) was added O-(7-Azabenzotriazol-1-yl)-N,N,N′,N′-tetramethyluronium hexafluorophosphate (433 mg, 1.14 mmol, 1.50 eq). And then the dimethylamine (619 mg, 7.60 mmol, 696 μL, 10.0 eq, hydrogen chloride) and N,N-diisopropylethylamine (1.30 g, 9.90 mmol, 1.72 mL, 13.0 eq) was added dropwise at −10° C. The mixture was stirred at 25° C. for 12 h. The reaction mixture was diluted with water (10.0 mL) and extracted with ethyl acetate (3×30.0 mL). The organic phase was separated, washed with brine (3×10.0 mL), dried over sodium sulfate, filtered and concentrated under reduced pressure to give a residue. The residue was purified by column chromatography (SiO₂, petroleum ether/ethyl acetate=10/1 to 3/1) and concentrated to afford tert-butyl (S)-2-(dimethylcarbamoyl)indoline-1-carboxylate (180 mg, 620 mol, 82% yield) as colorless oil.

¹H NMR (400 MHz, DMSO-d₆) δ=7.72 (br d, J=7.9 Hz, 1H), 7.13 (br d, J=8.3 Hz, 2H), 6.93-6.87 (m, 1H), 5.26-5.20 (m, 1H), 3.07 (s, 3H), 2.86 (s, 3H), 2.82 (br s, 2H), 1.39 (s, 9H). MS (ESI) m/z 191 [M−99]⁺

Step 2. A mixture of tert-butyl (S)-2-(dimethylcarbamoyl)indoline-1-carboxylate (80.0 mg, 276 mol, 1.00 eq) in dichloromethane (2.00 mL) was added trifluoroacetic acid (768 mg, 6.73 mmol, 0.500 mL, 24.4 eq). The mixture was stirred at 25° C. for 2 h. The mixture was concentrated under reduced pressure to afford (S)—N,N-dimethylindoline-2-carboxamide (80.0 mg, crude) as a colorless solid.

Step 3. A mixture of (S)—N,N-dimethylindoline-2-carboxamide (80.0 mg, 421 mol, 1.00 eq) and triethylamine (213 mg, 2.10 mmol, 293 μL, 5.00 eq) in dimethyl formamide (2.00 mL) was added phenyl N-[2-(2,6-dioxo-3-piperidyl)-1-oxo-isoindolin-5-yl]carbamate (144 mg, 378 mol, 0.900 eq). The mixture was stirred at 50° C. for 2 h. The mixture was filtered to afford a residue. The residue was purified by pre-HPLC (Welch ultimate C18 150*25 mm*7 um; mobile phase: [water (formic acid)-acetonitrile]; B %: 12%-42%, 15 min) and lyoplihization to afford (2S)-N1-(2-(2,6-dioxopiperidin-3-yl)-1-oxoisoindolin-5-yl)-N2,N2-dimethylindoline-1,2-dicarboxamide (25.0 mg, 52.6 mol, 13% yield, 96% purity) as a white solid. ¹H NMR (400 MHz, DMSO-d₆) δ=11.06-10.88 (m, 1H), 8.95 (s, 1H), 7.81 (d, J=7.9 Hz, 1H), 7.76 (br s, 1H), 7.63 (d, J=8.4 Hz, 1H), 7.52-7.46 (m, 1H), 7.19-7.12 (m, 2H), 6.95-6.86 (m, 1H), 5.74-5.66 (m, 1H), 5.12-5.01 (m, 1H), 4.46-4.38 (m, 1H), 4.32-4.25 (m, 1H), 3.71-3.61 (m, 1H), 3.11 (s, 3H), 3.05-2.91 (m, 2H), 2.81 (s, 3H), 2.68-2.61 (m, 1H), 2.38-2.31 (m, 1H), 2.06-1.94 (m, 1H). MS (ESI) m/z 476 [M+H]⁺

### Example 80. Synthesis of Compound 302

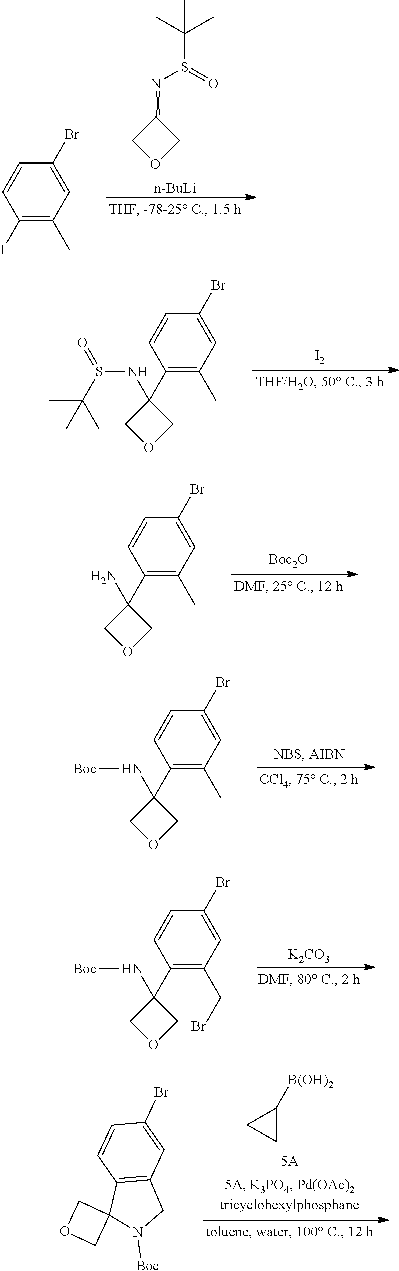

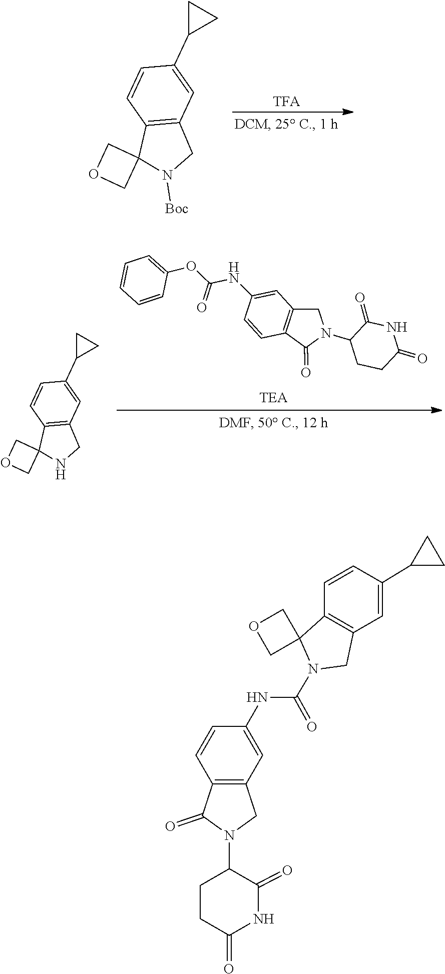 [SMILES: CC(C)(C)OC(=O)N1Cc2cc(C3CC3)ccc2C12COC2 ; c1cc2c(cc1C1CC1)CNC21COC1 ; O=C1CCC(N2Cc3cc(NC(=O)Oc4ccccc4)ccc3C2=O)C(=O)N1 ; O=C1CCC(N2Cc3cc(NC(=O)N4Cc5cc(C6CC6)ccc5C45COC5)ccc3C2=O)C(=O)N1 ; "TFA"]

Step 1. To a mixture of 4-bromo-1-iodo-2-methylbenzene (5.00 g, 16.8 mmol, 1.00 eq) in tetrahydrofuran (50.0 mL) was added n-butyllithium (2.50 M, 6.74 mL, 1.00 eq) at −78° C. under nitrogen atmosphere. The mixture was stirred at −78° C. for 0.5 h. The mixture was added 2-methyl-N-(oxetan-3-ylidene)propane-2-sulfinamide (2.66 g, 15.1 mmol, 0.900 eq) at −78° C., and then the mixture was stirred at −78° C. for 1 h under nitrogen atmosphere. The reaction mixture was quenched by addition saturated ammonium chloride (200 mL) aqueous solution, then the mixture was stirred at 0° C. for 0.5 h. The diluted reaction mixture was extracted with ethyl acetate (3×200 mL), washed with brine (200 mL), dried over anhydrous sodium sulfate, filtered via vacuum filtration, and concentrated in vacuum. The residue was purified by column chromatography (SiO2, Petroleum ether/Ethyl acetate=20/1 to 0/1) to give N-(3-(4-bromo-2-methylphenyl)oxetan-3-yl)-2-methylpropane-2-sulfinamide (2.60 g, 7.51 mmol, 44% yield) as a yellow solid. ¹H NMR (400 MHz, DMSO-d₆) δ=7.44-7.37 (m, 2H), 7.20 (d, J=8.6 Hz, 1H), 6.33 (s, 1H), 5.21 (d, J=6.8 Hz, 1H), 4.97-4.93 (m, 1H), 4.89 (d, J=6.5 Hz, 2H), 2.08 (s, 3H), 1.04 (s, 9H).

Step 2. To a mixture of N-(3-(4-bromo-2-methylphenyl)oxetan-3-yl)-2-methylpropane-2-sulfinamide (1.40 g, 4.04 mmol, 1.00 eq) in tetrahydrofuran (8.00 mL) and water (2.00 mL) was added iodine (513 mg, 2.02 mmol, 407 μL, 0.500 eq). The mixture was stirred at 50° C. for 3 h. The reaction mixture was diluted with saturated sodium bicarbonate solution (100 mL) and extracted with ethyl acetate (3×150 mL). The combined organic layers were washed with brine (150 mL), dried over anhydrous sodium sulfate, and concentrated under reduced pressure to give a residue. The residue was purified by column chromatography (SiO2, Petroleum ether/Ethyl acetate=20/1 to 0/1) to give 3-(4-bromo-2-methylphenyl)oxetan-3-amine (920 mg, crude) as yellow oil. ¹H NMR (400 MHz, DMSO-d₆) δ=7.40-7.30 (m, 2H), 7.04 (d, J=8.3 Hz, 1H), 4.98 (d, J=6.4 Hz, 2H), 4.54 (d, J=6.4 Hz, 2H), 3.06 (br d, J=2.1 Hz, 2H), 2.17 (s, 3H).

Step 3. To a mixture of 3-(4-bromo-2-methylphenyl)oxetan-3-amine (920 mg, 3.80 mmol, 1.00 eq) in dimethylformamide (1.00 mL) was added di-tert-butyl dicarbonate (1.66 g, 7.60 mmol, 1.75 mL, 2.00 eq). The mixture was stirred at 25° C. for 12 h. The reaction mixture was diluted with water (50 mL) and extracted with ethyl acetate (3×60 mL). The combined organic layers were washed with brine (50 mL), dried over anhydrous sodium sulfate, and concentrated under reduced pressure to give a residue. The residue was purified by column chromatography (SiO2, Petroleum ether/Ethyl acetate=20/1 to 5/1) to give tert-butyl (3-(4-bromo-2-methylphenyl)oxetan-3-yl)carbamate (1.00 g, 2.92 mmol, 76% yield) as a white solid. ¹H NMR (400 MHz, DMSO-d₆) δ=8.38-8.18 (m, 1H), 7.37-7.31 (m, 2H), 7.24 (d, J=8.8 Hz, 1H), 4.93 (d, J=6.5 Hz, 2H), 4.75 (d, J=6.5 Hz, 2H), 2.09 (s, 3H), 1.30 (br s, 9H).

Step 4. To a mixture of tert-butyl (3-(4-bromo-2-methylphenyl)oxetan-3-yl)carbamate (1.00 g, 2.92 mmol, 1.00 eq) in tetrachloromethane (10.0 mL) was added 2,2′-azobis(isobutyronitrile) (95.9 mg, 584 mol, 0.200 eq) and N-Bromosuccinimide (520 mg, 2.92 mmol, 1.00 eq). The mixture was stirred at 75° C. for 2 h. The reaction was concentrated under reduced pressure to give a residue. The residue was purified by column chromatography (SiO2, Petroleum ether/Ethyl acetate=1/0 to 5/1) to give tert-butyl (3-(4-bromo-2-(bromomethyl)phenyl)oxetan-3-yl)carbamate (560 mg, crude) as a white solid. ¹H NMR (400 MHz, DMSO-d₆) δ=8.31 (br d, J=10.3 Hz, 1H), 7.63 (d, J=1.6 Hz, 1H), 7.55-7.44 (m, 1H), 7.30 (d, J=8.4 Hz, 1H), 5.01 (d, J=6.8 Hz, 2H), 4.79 (d, J=6.9 Hz, 2H), 4.53 (s, 2H), 1.30 (br s, 9H).

Step 5. To a mixture of tert-butyl (3-(4-bromo-2-(bromomethyl)phenyl)oxetan-3-yl)carbamate (240 mg, 569 mol, 1.00 eq) in N,N-dimethylformamide (2.00 mL) was added potassium carbonate (126 mg, 911 mol, 1.60 eq). The mixture was stirred at 80° C. for 2 h. The reaction mixture was diluted with water (20 mL) and extracted with ethyl acetate (3×30 mL). The combined organic layers were washed with brine (100 mL), dried over sodium sulfate, and concentrated under reduced pressure to give a residue. The residue was purified by column chromatography (SiO2, Petroleum ether/Ethyl acetate=1/0 to 5/1) to give tert-butyl 5-bromospiro[isoindoline-1,3′-oxetane]-2-carboxylate (130 mg, 382 mol, 67% yield) as a yellow solid. ¹H NMR (400 MHz, DMSO-d₆) δ=7.71-7.67 (m, 1H), 7.66-7.61 (m, 1H), 7.58 (s, 1H), 5.51 (br d, J=5.4 Hz, 1H), 5.36 (br d, J=5.8 Hz, 1H), 4.57 (br d, J=7.4 Hz, 3H), 4.48 (br d, J=5.3 Hz, 1H), 1.57-1.49 (m, 9H).

Step 6. To a mixture of tert-butyl 5-bromospiro[isoindoline-1,3′-oxetane]-2-carboxylate (230 mg, 676 mol, 1.00 eq) in toluene (3.00 mL) and water (0.300 mL) was added cyclopropylboronic acid (232 mg, 2.70 mmol, 4.00 eq), potassium phosphate (430 mg, 2.03 mmol, 3.00 eq), tricyclohexylphosphane (37.9 mg, 135 mol, 43.8 μL, 0.200 eq) and palladium acetate (15.1 mg, 67.6 mol, 0.100 eq) under nitrogen atmosphere. The mixture was stirred at 100° C. for 12 h under nitrogen atmosphere. The reaction mixture was diluted with water (20 mL) and extracted with ethyl acetate (3×30 mL). The combined organic layers were washed with brine (100 mL), dried over anhydrous sodium sulfate and concentrated under reduced pressure to give a residue. The residue was purified by column chromatography (SiO2, Petroleum ether/Ethyl acetate=1/0 to 1/1) to give tert-butyl 5-cyclopropylspiro[isoindoline-1,3′-oxetane]-2-carboxylate (168 mg, 557 mol, 82% yield) as a yellow solid. ¹H NMR (400 MHz, DMSO-d₆) δ=7.58 (dd, J=2.6, 7.9 Hz, 1H), 7.15 (br d, J=7.6 Hz, 1H), 7.02 (s, 1H), 5.48 (br d, J=5.4 Hz, 1H), 5.34 (br d, J=5.8 Hz, 1H), 4.59-4.44 (m, 4H), 1.97-1.90 (m, 1H), 1.57-1.47 (m, 9H), 0.99-0.91 (m, 2H), 0.70-0.63 (m, 2H).

Step 7. To a mixture of tert-butyl 5-cyclopropylspiro[isoindoline-1,3′-oxetane]-2-carboxylate (168 mg, 557 mol, 1.00 eq) in dichloromethane (2.50 mL) was added trifluoroacetic acid (767 mg, 6.73 mmol, 0.500 mL, 12.0 eq). The mixture was stirred at 25° C. for 1 h. The reaction was concentrated under reduced pressure to give 5-cyclopropylspiro[isoindoline-1,3′-oxetane](105 mg, crude) as orange oil. MS (ESI) m/z 202.3 [M+H]⁺

Step 8. To a mixture of 5-cyclopropylspiro[isoindoline-1,3′-oxetane](100 mg, 496 mol, 1.00 eq) in N,N-dimethylformamide (2.00 mL) was added phenyl (2-(2,6-dioxopiperidin-3-yl)-1-oxoisoindolin-5-yl)carbamate (188 mg, 496 mol, 1.00 eq) and triethylamine (150 mg, 1.49 mmol, 207 μL, 3.00 eq). The mixture was stirred at 50° C. for 12 h. The mixture was filtered to collect the filtrate. The filtarte was purified by prep-HPLC (column: Welch ultimate C18 150*25 mm*7 um; mobile phase: [water (formic acid)-ACN]; gradient:25%-55% B over 10 min) and lyophilized to give 5-cyclopropyl-N-(2-(2,6-dioxopiperidin-3-yl)-1-oxoisoindolin-5-yl)spiro[isoindoline-1,3′-oxetane]-2-carboxamide (88.07 mg, 181 mol, 36% yield, 100% purity) as a white solid. ¹H NMR (400 MHz, DMSO-d₆) δ=10.97 (s, 1H), 8.70 (s, 1H), 7.99 (s, 1H), 7.69-7.60 (m, 3H), 7.18 (d, J=8.0 Hz, 1H), 7.05 (s, 1H), 5.65 (d, J=5.4 Hz, 2H), 5.09 (dd, J=5.1, 13.3 Hz, 1H), 4.83 (s, 2H), 4.50 (d, J=5.3 Hz, 2H), 4.47-4.39 (m, 1H), 4.33-4.26 (m, 1H), 2.98-2.83 (m, 1H), 2.66-2.57 (m, 1H), 2.39 (br d, J=8.8 Hz, 1H), 2.05-1.97 (m, 2H), 1.01-0.94 (m, 2H), 0.73-0.67 (m, 2H). MS (ESI) m/z 487.3 [M+H]⁺

### Example 81. Synthesis of Compound 306

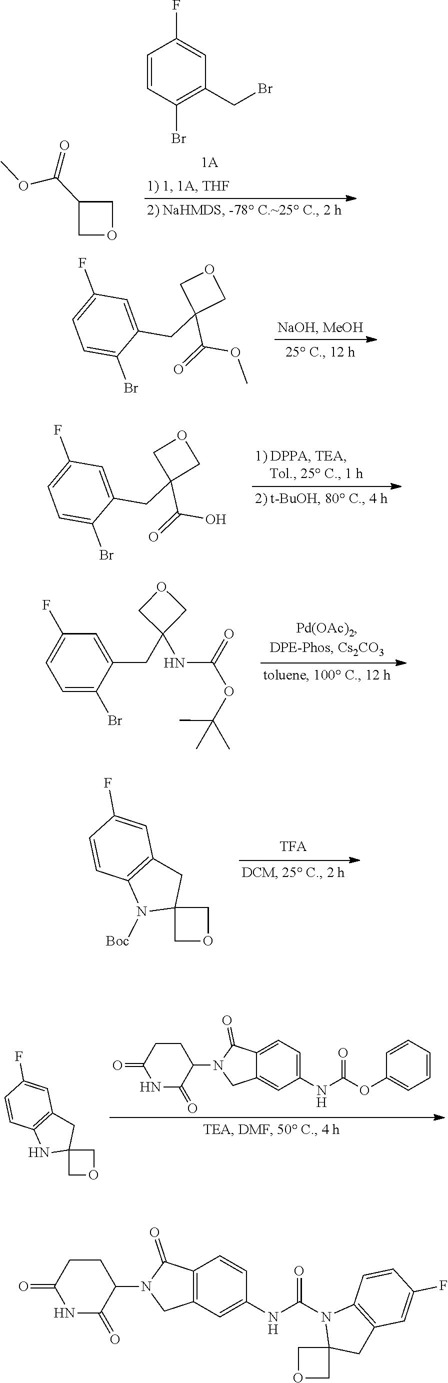

Step 1. To a mixture of methyl oxetane-3-carboxylate (400 mg, 3.44 mmol, 1.00 eq) and 1-bromo-2-(bromomethyl)-4-fluorobenzene (1.38 g, 5.17 mmol, 1.50 eq) in tetrahydrofuran (3.00 mL) was added sodium bis(trimethylsilyl)amide (1.00 M, 4.48 mL, 1.30 eq) at −30° C. under nitrogen. The mixture was stirred at −30° C. for 30 min. Then the mixture was stirred at 25° C. for 1 h under nitrogen. The reaction mixture was quenched by addition saturated ammonium chloride (20 mL) aquenous solution, then the mixture was stirred at 0° C. for 0.5 h. The diluted reaction mixture was extracted with ethyl acetate (3×50 mL), washed with brine (50 mL), dried over sodium sulfate, filtered via vacuum filtration, and concentrated in vacuum. The residue was purified by column chromatography (SiO2, Petroleum ether/Ethyl acetate=1/0 to 20/1) and concentrated in vacuum to give methyl 3-(2-bromo-5-fluorobenzyl)oxetane-3-carboxylate (550 mg, 1.81 mmol, 26% yield) as yellow oil.

¹H NMR (400 MHz, DMSO-d₆) δ=7.67 (dd, J=5.6, 8.8 Hz, 1H), 7.11 (dt, J=3.0, 8.5 Hz, 1H), 7.03 (dd, J=3.1, 9.8 Hz, 1H), 4.76 (d, J=6.3 Hz, 2H), 4.61 (d, J=6.4 Hz, 2H), 3.67 (s, 3H), 3.39 (s, 2H).

Step 2. To a mixture of methyl 3-(2-bromo-5-fluorobenzyl)oxetane-3-carboxylate (740 mg, 2.44 mmol, 1.00 eq) in ethanol (12.0 mL) was added sodium hydroxide (1.00 M, 6.96 mL, 2.85 eq). The mixture was stirred at 25° C. for 12 h. The reaction mixture was poured into water (10.0 mL) and adjust Ph=3-4 with 1M hydrochloric acid. The aqueous phase was extracted with ethyl acetate (3×10 mL). The combined organic phase was washed with brine (10 mL), dried over anhydrous sodium sulfate, filtered and concentrated in vacuum to give 3-(2-bromo-5-fluorobenzyl)oxetane-3-carboxylic acid (630 mg, 2.18 mmol, 89% yield) as a white solid. ¹H NMR (400 MHz, DMSO-d₆) δ=13.09 (br s, 1H), 7.69 (dd, J=5.6, 8.8 Hz, 1H), 7.11 (dt, J=3.0, 8.5 Hz, 1H), 6.95 (dd, J=3.0, 9.9 Hz, 1H), 4.77 (d, J=6.3 Hz, 2H), 4.55 (d, J=6.1 Hz, 2H), 3.36 (s, 2H).

Step 3. To a solution of 3-(2-bromo-5-fluorobenzyl)oxetane-3-carboxylic acid (300 mg, 1.04 mmol, 1.00 eq), molecular sieves, 13×(50.0 mg, 1.04 mmol, 1.00 eq) and triethylamine (315 mg, 3.11 mmol, 433 μL, 3.00 eq) in toluene (3.00 mL) was added diphenylphosphoryl azide (428 mg, 1.56 mmol, 336 μL, 1.50 eq) dropwise at 0° C., the mixture was stirred at 80° C. for 1 h. And then the mixture was added tertiary butanol (3.88 g, 52.3 mmol, 5.00 mL, 50.4 eq) and stirred at 80° C. for 4 h under nitrogen. The mixture was concentrated in vacuum. The residue was purified by column chromatography (SiO2, Petroleum ether/Ethyl acetate=1/0 to 1/1) and concentrated in vacuum to give tert-butyl (3-(2-bromo-5-fluorobenzyl)oxetan-3-yl)carbamate (210 mg, 583 mol, 56% yield) as a white solid. ¹H NMR (400 MHz, DMSO-d₆) δ=7.65 (dd, J=5.6, 8.8 Hz, 1H), 7.59 (br s, 1H), 7.09 (dt, J=3.1, 8.5 Hz, 1H), 7.03 (dd, J=2.9, 10.0 Hz, 1H), 4.60-4.52 (m, 2H), 4.52-4.45 (m, 2H), 3.39 (s, 2H), 1.38 (br s, 9H).

Step 4. To a mixture of tert-butyl (3-(2-bromo-5-fluorobenzyl)oxetan-3-yl)carbamate (210 mg, 583 mol, 1.00 eq) in toluene (5.00 mL) was added cesium carbonate (380 mg, 1.17 mmol, 2.00 eq), bis(2-diphenylphosphinophenyl)ether (62.8 mg, 116 mol, 0.200 eq) and palladium(ii) acetate (13.1 mg, 58.3 mol, 0.100 eq) under nitrogen. The mixture was stirred at 100° C. for 12 h under nitrogen. The reaction mixture was diluted with water (10 mL) and extracted with ethyl acetate (3×20 mL). The combined organic layers were washed with brine (10 mL), dried over sodium sulfate, filtered and concentrated under reduced pressure to give a residue. The residue was purified by column chromatography (SiO2, Petroleum ether/Ethyl acetate=1/0 to 1/1) and concentrated in vacuum to give tert-butyl 5-fluorospiro[indoline-2,3′-oxetane]-1-carboxylate (115 mg, 411 mol, 70% yield) as a white solid. ¹H NMR (400 MHz, DMSO-d₆) δ=7.78-7.50 (m, 1H), 7.07 (dd, J=2.5, 8.3 Hz, 1H), 6.96 (dt, J=2.6, 9.1 Hz, 1H), 5.28 (d, J=6.0 Hz, 2H), 4.50 (d, J=6.1 Hz, 2H), 3.65 (s, 2H), 1.58 (s, 9H).

Step 5. To a mixture of tert-butyl 5-fluorospiro[indoline-2,3′-oxetane]-1-carboxylate (115 mg, 411 mol, 1.00 eq) in dichloromethane (2.50 mL) was added trifluoroacetic acid (46.9 mg, 411 mol, 30.6 μL, 1.00 eq). The mixture was stirred at 25° C. for 2 h. The mixture was concentrated in vacuum to give 5-fluorospiro[indoline-2,3′-oxetane](60 mg, crude) as yellow oil. MS (ESI) m/z. 180.0 [M+H]⁺

Step 6. To a mixture of 5-fluorospiro[indoline-2,3′-oxetane](50.0 mg, 279 mol, 1.00 eq) and phenyl N-[2-(2,6-dioxo-3-piperidyl)-1-oxo-isoindolin-5-yl]carbamate (84.7 mg, 223 mol, 0.800 eq) in dimethyformamide (0.500 mL) was added triethylamine (84.7 mg, 837 mol, 116 μL, 3.00 eq). The mixture was stirred at 50° C. for 4 h. The mixture was filtered to give a filtrate. The filtrate was purified by prep-HPLC (column: Phenomenex luna C18 150*25 mm*10 um; mobile phase: [water (FA)-ACN]; gradient:24%-54% B over 10 min) and lyophilized. The compound was purified by prep-HPLC (column: Phenomenex luna C18 150*25 mm*10 um; mobile phase: [water (FA)-ACN]; gradient:25%-55% B over 10 min) and lyophilized again to give N-(2-(2,6-dioxopiperidin-3-yl)-1-oxoisoindolin-5-yl)-5-fluorospiro[indoline-2,3′-oxetane]-1-carboxamide (10.87 mg, 23.2 mol, 8% yield, 99% purity) as a white solid. ¹H NMR (400 MHz, DMSO-d₆) δ=10.98 (s, 1H), 9.88 (s, 1H), 7.87 (s, 1H), 7.67 (d, J=8.3 Hz, 1H), 7.56 (d, J=8.5 Hz, 1H), 7.32-7.26 (m, 1H), 7.18-7.12 (m, 1H), 7.04-6.97 (m, 1H), 5.32 (d, J=6.3 Hz, 2H), 5.10 (dd, J=5.4, 13.1 Hz, 1H), 4.64 (br d, J=6.4 Hz, 2H), 4.48-4.38 (m, 1H), 4.37-4.23 (m, 1H), 3.56 (s, 2H), 2.99-2.85 (m, 1H), 2.58 (br d, J=3.3 Hz, 1H), 2.37 (br s, 1H), 2.04-1.98 (m, 1H). MS (ESI) m/z. 465.2 [M+H]⁺

### Example 82. Synthesis of Compound 322

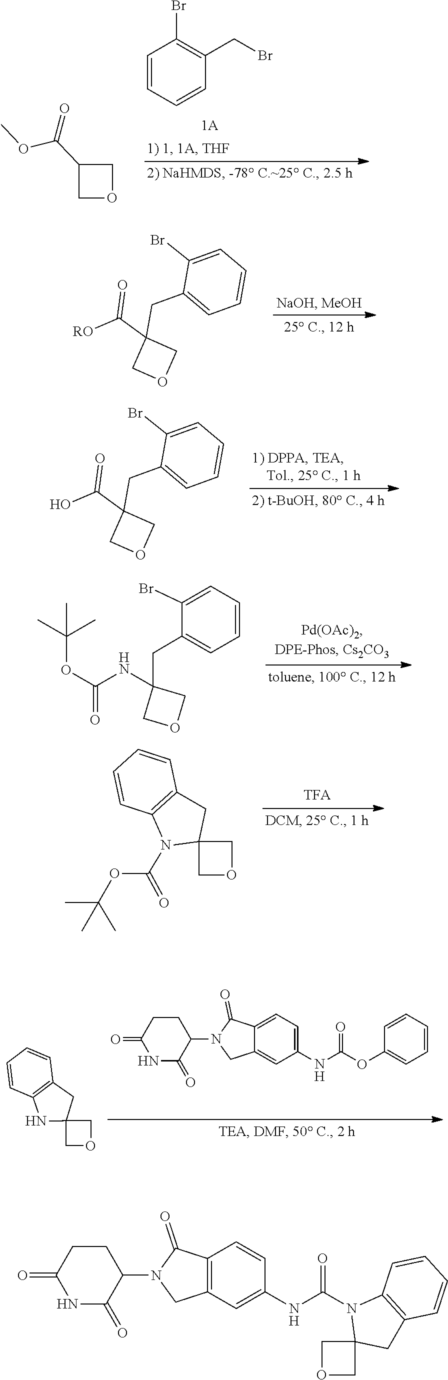

Step 1. To a solution of methyl oxetane-3-carboxylate (500 mg, 4.31 mmol, 1.00 eq) and 1-bromo-2-(bromomethyl)benzene (1.29 g, 5.17 mmol, 1.20 eq) in tetrahydrofuran (6.00 mL) wad added sodium bis(trimethylsilyl)amide (1.00 M, 5.60 mL, 1.30 eq) at −70° C. under nitrogen atmosphere, the mixture was stirred at −70° C. for 0.5 h and stirred at 25° C. for 2 h. The reaction mixture was quenched with saturated ammonium chloride (5.00 mL) and poured into water (80 mL), then extracted with ethyl acetate (3×40 mL). The combined organic phase was washed with brine (80 mL), dried over anhydrous sodium sulfate, filtered and concentrated in vacuum to give a residue. The residue was purified by silica gel chromatograph (Petroleum ether/Ethyl acetate=1/0 to 20/1) to afford methyl 3-(2-bromobenzyl)oxetane-3-carboxylate (600 mg, 2.10 mmol, 48% yield) as colorless oil. ¹H NMR (400 MHz, DMSO-d₆) δ=7.62 (dd, J=0.9, 8.0 Hz, 1H), 7.37-7.28 (m, 1H), 7.19 (dt, J=1.6, 7.7 Hz, 1H), 7.10 (dd, J=1.4, 7.6 Hz, 1H), 4.76 (d, J=6.3 Hz, 2H), 4.60 (d, J=6.3 Hz, 2H), 3.66 (s, 3H), 3.40 (s, 2H).

Step 2. To a solution of methyl 3-(2-bromobenzyl)oxetane-3-carboxylate (580 mg, 2.03 mmol, 1.00 eq) in methanol (10.0 mL) was added sodium hydroxide (1.00 M, 5.80 mL, 2.85 eq), the mixture was stirred at 25° C. for 12 h. the reaction mixture was poured into water (80.0 mL) and adjust pH=1-2 with 1M hydrochloric acid. The aqueous phase was extracted with ethyl acetate (3×40 mL). The combined organic phase was washed with brine (100 mL), dried over anhydrous sodium sulfate, filtered and concentrated in vacuum to give 3-(2-bromobenzyl)oxetane-3-carboxylic acid (500 mg, 1.84 mmol, 90% yield) as a white solid. ¹H NMR (400 MHz, DMSO-d₆) δ=13.47-12.33 (m, 1H), 7.58 (dd, J=0.9, 7.9 Hz, 1H), 7.33-7.19 (m, 1H), 7.14 (dt, J=1.5, 7.7 Hz, 1H), 7.01 (dd, J=1.3, 7.6 Hz, 1H), 4.72 (d, J=6.1 Hz, 2H), 4.49 (d, J=6.0 Hz, 2H), 3.31 (s, 2H).

Step 3. To a solution of 3-(2-bromobenzyl)oxetane-3-carboxylic acid (450 mg, 1.66 mmol, 1.00 eq) and triethylamine (184 mg, 1.83 mmol, 254 μL, 1.10 eq) in toluene. (4.00 mL) was added diphenylphosphoryl azide (456 mg, 1.66 mmol, 358 μL, 1.00 eq) dropwise at 0° C., the mixture was stirred at 25° C. for 1 h, then tertiary butanol (4.00 mL) was added, the mixture was stirred at 80° C. for 4 h. The reaction mixture was concentrated to give a residue. The residue was purified by silica gel chromatography (Petroleum ether/Ethyl acetate=1/0 to 10/1) to afford tert-butyl (3-(2-bromobenzyl)oxetan-3-yl)carbamate (280 mg, 818 mol, 49% yield) as a yellow solid. ¹H NMR (400 MHz, DMSO-d₆) δ=7.66-7.59 (m, 1H), 7.55 (br s, 1H), 7.37-7.31 (m, 1H), 7.25-7.14 (m, 2H), 4.55 (br d, J=6.0 Hz, 2H), 4.51-4.45 (m, 2H), 3.38 (s, 2H), 1.40 (br s, 9H).

Step 4. To a solution of tert-butyl (3-(2-bromobenzyl)oxetan-3-yl)carbamate (280 mg, 818 mol, 1.00 eq), [2-(2-diphenylphosphanylphenoxy)phenyl]-diphenyl-phosphane (88.0 mg, 163 mol, 0.200 eq) and cesium carbonate (533 mg, 1.64 mmol, 2.00 eq) in toluene (5.00 mL) was added palladium acetate (18.4 mg, 81.8 mol, 0.100 eq), the mixture was stirred at 100° C. for 12 h. The reaction mixture was poured into water (80.0 mL) and extracted with ethyl acetate (3×40 mL). The combined organic phase was washed with brine (80 mL), dried over anhydrous sodium sulfate, filtered and concentrated in vacuum to give a residue. The residue was purified by silica gel chromatography (Petroleum ether/Ethyl acetate=100/1 to 20/1) to afford tert-butyl spiro[indoline-2,3′-oxetane]-1-carboxylate (130 mg, 497 mol, 60% yield) as yellow oil. ¹H NMR (400 MHz, DMSO-d₆) δ=7.68 (br d, J=6.9 Hz, 1H), 7.25-7.10 (m, 2H), 7.01-6.90 (m, 1H), 5.31 (d, J=6.1 Hz, 2H), 4.52 (d, J=6.1 Hz, 2H), 3.66 (s, 2H), 1.60 (s, 9H). MS (ESI) m/z 206.0 [M+H−56]⁺

Step 5. To a solution of tert-butyl spiro[indoline-2,3′-oxetane]-1-carboxylate (60.0 mg, 229 mol, 1.00 eq) in dichloromethane (2.00 mL) was added trifluoroacetic acid (614 mg, 5.39 mmol, 400 μL, 23.4 eq), the mixture was stirred at 25° C. for 1 h. The reaction mixture was concentrated to give spiro[indoline-2,3′-oxetane](35.0 mg, 217 mol, 94% yield) as yellow oil.

Step 6. To a solution of spiro[indoline-2,3′-oxetane](35.0 mg, 217 mol, 1.00 eq) and triethylamine (87.9 mg, 868 mol, 120 μL, 4.00 eq) in dimethylformamide (2.00 mL) was added phenyl (2-(2,6-dioxopiperidin-3-yl)-1-oxoisoindolin-5-yl)carbamate (65.0 mg, 173 mol, 0.800 eq), the mixture was stirred at 50° C. for 2 h. The reaction mixture was filtered to give a filtrate. the filtrate was purified by Prep-HPLC (column: Phenomenex luna C18 150*25 mm*10 um; mobile phase: [water (FA)-ACN]; gradient:21%-51% B over 10 min) to afford N-(2-(2,6-dioxopiperidin-3-yl)-1-oxoisoindolin-5-yl)spiro[indoline-2,3′-oxetane]-1-carboxamide (10.2 mg, 22.8 mol, 10% yield as white solid. ¹H NMR (400 MHz, DMSO-d₆) δ=10.98 (s, 1H), 9.92 (s, 1H), 7.89 (s, 1H), 7.71-7.65 (m, 1H), 7.59 (dd, J=1.7, 8.3 Hz, 1H), 7.32-7.24 (m, 2H), 7.22-7.15 (m, 1H), 6.96 (t, J=7.3 Hz, 1H), 5.36 (d, J=6.3 Hz, 2H), 5.10 (dd, J=5.1, 13.4 Hz, 1H), 4.61 (d, J=6.3 Hz, 2H), 4.48-4.42 (m, 1H), 4.35-4.28 (m, 1H), 3.56 (s, 2H), 3.00-2.87 (m, 1H), 2.61 (br dd, J=2.1, 15.7 Hz, 1H), 2.40 (br dd, J=4.5, 12.9 Hz, 1H), 2.09-1.94 (m, 1H). MS (ESI) m/z 447.0 [M+H]⁺

### Example 83. Synthesis of Compound 305

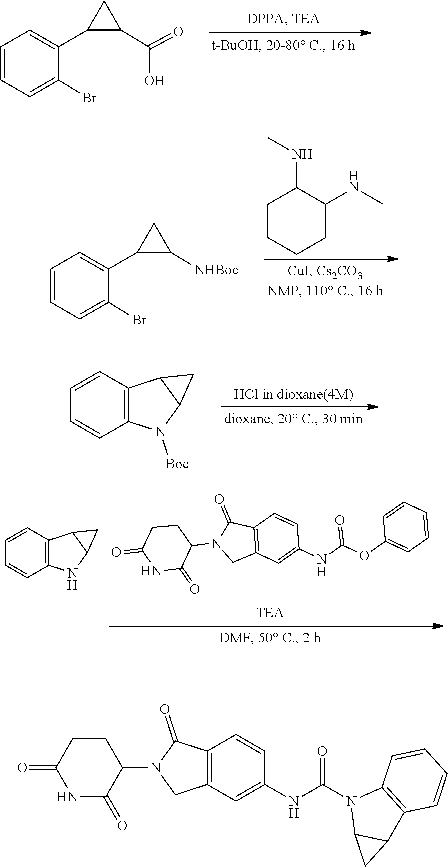

Step 1. To a solution of 2-(2-bromophenyl)cyclopropane-1-carboxylic acid (2.00 g, 8.30 mmol, 1.00 eq) in tertiary butanol (20.0 mL) was added triethylamine (923 mg, 9.13 mmol, 1.27 mL, 1.10 eq) and diphenyl phosphoryl azide (2.51 g, 9.13 mmol, 1.97 mL, 1.10 eq). The mixture was stirred at 20° C. for 2 hours under nitrogen and then stirred at 80° C. for 14 hours under nitrogen. The reaction mixture was quenched by addition water 50.0 mL at 20° C., and then extracted with ethyl acetate (2×50.0 mL). The combined organic layers were washed with brine (2×50.0 mL), dried over anhydrous sodium sulfate, filtered and concentrated under reduced pressure to give a residue. The residue was purified by column chromatography (silicon dioxide, petroleum ether/ethyl acetate=20/1 to 5/1) to afford tert-butyl (2-(2-bromophenyl)cyclopropyl)carbamate (900 mg, 2.76 mmol, 33% yield) as yellow solid. MS (ESI) m/z 256.1 [M+H−56]⁺

Step 2. A mixture of tert-butyl (2-(2-bromophenyl)cyclopropyl)carbamate (200 mg, 641 mol, 1.00 eq), cuprous iodide (122 mg, 641 mol, 1.00 eq), cesium carbonate (626 mg, 1.92 mmol, 3.00 eq) and N¹,N²-dimethylcyclohexane-1,2-diamine (137 mg, 961 mol, 1.50 eq) in 1-methyl-2-pyrrolidone (1.00 mL) was degassed and purged with nitrogen for 3 times, and then the mixture was stirred at 110° C. for 16 hours under nitrogen atmosphere. The reaction mixture was partitioned between water 20.0 mL and ethyl acetate 30.0 mL. The organic phase was separated, washed with brine (2×15.0 mL), dried over anhydrous sodium sulfate, filtered and concentrated under reduced pressure to give a residue. The residue was purified by column chromatography (silicon dioxide, petroleum ether/ethyl acetate=1/0 to 10/1) to afford tert-butyl 1a, 6b-dihydrocyclopropa[b]indole-2(1H)-carboxylate (100 mg, 432 mol, 67% yield) as a yellow oil. ¹H NMR (400 MHz, CDCl₃) δ=8.05-7.41 (m, 1H), 7.31 (d, J=7.6 Hz, 1H), 7.15 (t, J=7.6 Hz, 1H), 6.95 (dt, J=0.8, 7.4 Hz, 1H), 4.39-4.01 (m, 1H), 2.61 (ddd, J=4.0, 6.6, 8.6 Hz, 1H), 1.70-1.58 (m, 9H), 1.06 (br d, J=2.0 Hz, 1H), 0.29-0.20 (m, 1H). MS (ESI) m/z 176.2 [M+H−56]⁺

Step 3. To a solution of tert-butyl 1a, 6b-dihydrocyclopropa[b]indole-2(1H)-carboxylate (90.0 mg, 389 mol, 1.00 eq) in dioxane (10.0 mL) was added hydrochloric acid/dioxane (4.00 M, 2.00 mL, 20.6 eq). The mixture was stirred at 20° C. for 30 min. The reaction mixture was concentrated under reduced pressure to afford 1,1a, 2,6b-tetrahydrocyclopropa[b]indole (51.0 mg, 389 mol, 99% yield) as a brown solid. ¹H NMR (400 MHz, DMSO-d₆) δ=7.27 (d, J=7.6 Hz, 1H), 7.06-6.94 (m, 1H), 6.89-6.76 (m, 2H), 3.64 (dt, J=2.4, 6.1 Hz, 1H), 2.67-2.55 (m, 1H), 1.00 (td, J=6.0, 8.4 Hz, 1H), 0.06-0.05 (m, 1H)

Step 4. To a solution of 1,1a, 2,6b-tetrahydrocyclopropa[b]indole (51.0 mg, 389 mol, 1.00 eq) and phenyl N-[2-(2,6-dioxo-3-piperidyl)-1-oxo-isoindolin-5-yl]carbamate (148 mg, 389 mol, 1.00 eq) in dimethyl formamide (1.00 mL) was added triethylamine (157 mg, 1.56 mmol, 216 μL, 4.00 eq). The mixture was stirred at 50° C. for 2 hours. The reaction mixture was filtered to afford the residue and the residue was purified by prep-HPLC (column: Welch Xtimate C18 150*25 mm*5 um; mobile phase: [water (formic acid)-acetonitrile]; gradient:25%-55% B over 9 min) to afford N-(2-(2,6-dioxopiperidin-3-yl)-1-oxoisoindolin-5-yl)-1a, 6b-dihydrocyclopropa[b]indole-2(1H)-carboxamide as a yellow solid. ¹H NMR (400 MHz, DMSO-d₆) δ=10.98 (s, 1H), 9.31 (s, 1H), 7.95 (s, 1H), 7.87 (d, J=8.0 Hz, 1H), 7.74-7.65 (m, 2H), 7.38 (d, J=6.8 Hz, 1H), 7.17-7.11 (m, 1H), 6.94 (dt, J=0.8, 7.4 Hz, 1H), 5.10 (dd, J=5.2, 13.3 Hz, 1H), 4.49-4.39 (m, 2H), 4.36-4.27 (m, 1H), 2.99-2.79 (m, 2H), 2.60 (br dd, J=2.0, 15.3 Hz, 1H), 2.43-2.31 (m, 1H), 2.05-1.95 (m, 1H), 1.19 (td, J=5.6, 8.6 Hz, 1H), 0.30-0.23 (m, 1H). MS (ESI) m/z 417.3 [M+H]⁺

### Example 84. Synthesis of Compound 320

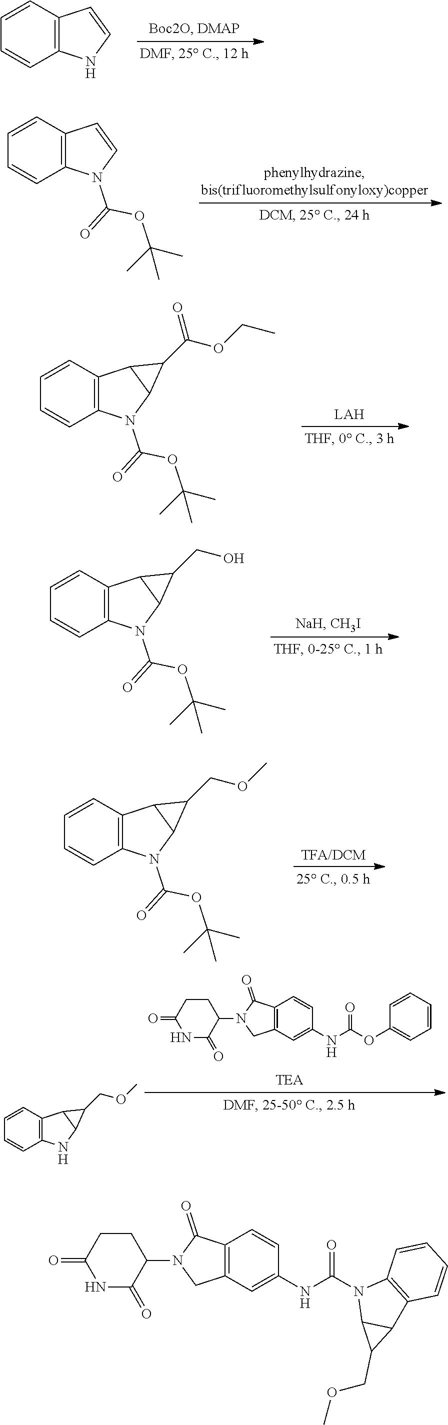

Step 1. To a mixture of 1H-indole (5.00 g, 42.7 mmol, 1.00 eq) in dimethyformamide (50.0 mL) was added 4-dimethylaminopyridine (521 mg, 4.27 mmol, 0.100 eq) and di-tert-butyldicarbonate (11.2 g, 51.2 mmol, 11.8 mL, 1.20 eq). The mixture was stirred at 25° C. for 12 h. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by column chromatography (SiO₂, Petroleum ether/Ethyl acetate=1/0 to 10/1) and concentrated in vacuum to give tert-butyl 1H-indole-1-carboxylate (8.40 g, 38.7 mmol, 90% yield) as yellow oil. ¹H NMR (400 MHz, DMSO-d₆) δ=8.07 (d, J=8.3 Hz, 1H), 7.64 (s, 1H), 7.60 (d, J=7.8 Hz, 1H), 7.35-7.27 (m, 1H), 7.25-7.18 (m, 1H), 6.69 (d, J=3.6 Hz, 1H), 1.61 (s, 9H).

Step 2. To a mixture of tert-butyl 1H-indole-1-carboxylate (2.00 g, 9.21 mmol, 1.00 eq) in dichloromethane (2.00 mL) was added bis(trifluoromethylsulfonyloxy)copper (333 mg, 921 umol, 0.100 eq), phenylhydrazine (99.6 mg, 921 umol, 90.5 uL, 0.100 eq) and ethyl 2-diazoacetate (2.10 g, 18.4 mmol, 1.93 mL, 2.00 eq). The mixture was stirred at 25° C. for 12 h. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by column chromatography (SiO2, Petroleum ether/Ethyl acetate=20/0 to 1/1) and concentrated in vacuum to give. 2-(tert-butyl) 1-methyl 1a, 6b-dihydrocyclopropa[b]indole-1,2(1H)-dicarboxylate (700 mg, 2.31 mmol, 25% yield) as brown oil. ¹H NMR (400 MHz, DMSO-d₆) δ=7.72-7.52 (m, 1H), 7.48 (d, J=7.4 Hz, 1H), 7.27-7.19 (m, 1H), 7.01 (dt, J=0.6, 7.5 Hz, 1H), 4.47 (dd, J=1.5, 6.8 Hz, 1H), 4.15-4.10 (m, 2H), 3.28 (dd, J=3.0, 6.8 Hz, 1H), 1.63 (s, 1H), 1.58-1.48 (m, 9H), 1.27-1.23 (m, 3H).

Step 3. To a mixture of 2-(tert-butyl) 1-ethyl 1a, 6b-dihydrocyclopropa[b]indole-1,2(1H)-dicarboxylate (450 mg, 1.48 mmol, 1.00 eq) in tetrahydrofuran (5.00 mL) was added lithium aluminum hydride (169 mg, 4.45 mmol, 3.00 eq) at 0° C. The mixture was stirred at 0° C. for 3 h under nitrogen. The reaction mixture was quenched by addition saturated sodium sulfate 10 water (5.00 mL) aqueous solution, then the mixture was stirred at 25° C. for 0.5 h. The diluted reaction mixture was extracted with ethyl acetate (3×50 mL), washed with brine (50 mL), dried over sodium sulfate, filtered via vacuum filtration, and concentrated in vacuum. The residue was purified by reversed phase (column: spherical C18, 20-45 um, 100 Å, SW 80, mobile phase: [water (0.1% Formic Acid)-acetonitrile) and lyophilized to give tert-butyl 1-(hydroxymethyl)-1a, 6b-dihydrocyclopropa[b]indole-2(1H)-carboxylate (20.0 mg, 76.5 mol, 5% yield) as a brown solid. ¹H NMR (400 MHz, DMSO-d₆) δ=7.74-7.50 (m, 1H), 7.33 (d, J=7.1 Hz, 1H), 7.12 (t, J=7.3 Hz, 1H), 6.92 (t, J=7.6 Hz, 1H), 4.69 (br t, J=5.4 Hz, 1H), 4.03 (br d, J=2.3 Hz, 1H), 3.57-3.40 (m, 2H), 2.62 (dd, J=3.3, 6.6 Hz, 1H), 1.52 (s, 9H), 0.61-0.51 (m, 1H).

Step 4. To a mixture of tert-butyl 1-(hydroxymethyl)-1a, 6b-dihydrocyclopropa[b]indole-2(1H)-carboxylate (60.0 mg, 229 mol, 1.00 eq) in tetrahydrofuran (1.00 mL) was added sodium hydride (13.8 mg, 3441 mol, 60% purity, 1.50 eq) and iodomethane (65.2 mg, 459 mol, 28.6 μL, 2.00 eq) at 0° C. The mixture was stirred at 25° C. for 1 h. The reaction mixture was quenched by addition saturated ammonium chloride (3 mL) aqueous solution. The diluted reaction mixture was extracted with ethyl acetate (3×5 mL), washed with brine (10 mL), dried over sodium sulfate, filtered via vacuum filtration, and concentrated in vacuum to give tert-butyl 1-(methoxymethyl)-1a, 6b-dihydrocyclopropa[b]indole-2(1H)-carboxylate (60 mg, crude) was obtained as yellow oil. MS (ESI) m/z. 573.5 [M+H]⁺

Step 5. To a mixture of tert-butyl 1-(methoxymethyl)-1a, 6b-dihydrocyclopropa[b]indole-2(1H)-carboxylate (50.0 mg, 181 mol, 1.00 eq) in dichloromethane (1.00 mL) was added trifluoroacetic acid (307 mg, 2.69 mmol, 0.200 mL, 14.8 eq). The mixture was stirred at 25° C. for 0.5 h. The reaction mixture was concentrated in vacuum to give 1-(methoxymethyl)-1,1a, 2,6b-tetrahydrocyclopropa[b]indole (30.0 mg, crude) as yellow oil. MS (ESI) m/z. 176.4 [M+H]⁺

Step 6. To a mixture of 1-(methoxymethyl)-1,1a, 2,6b-tetrahydrocyclopropa[b]indole (30.0 mg, 171 mol, 1.00 eq) and phenyl (2-(2,6-dioxopiperidin-3-yl)-1-oxoisoindolin-5-yl)carbamate (64.9 mg, 171 mol, 1.00 eq) in dimethyformamide (1.00 mL) was added triethylamine (51.9 mg, 513 mol, 71.5 μL, 3.00 eq). The mixture was stirred at 25° C. for 2 h. The mixture was stirred at 50° C. for 0.5 h. The mixture was filtered. The filtrate was purified by Prep-HPLC (column: Phenomenex luna C18 150*25 mm*10 um; mobile phase: [water (formic acid)-acetonitrile]; B %: 27%-57%, 10 min) and lyophilized to give N-(2-(2,6-dioxopiperidin-3-yl)-1-oxoisoindolin-5-yl)-1-(methoxymethyl)-1a, 6b-dihydrocyclopropa[b]indole-2(1H)-carboxamide (3.82 mg, 7.71 mol, 4% yield, 93% purity) as a white solid. ¹H NMR (400 MHz, DMSO-d₆) δ=11.06-10.89 (m, 1H), 9.18 (s, 1H), 7.97-7.81 (m, 2H), 7.73-7.61 (m, 2H), 7.38 (d, J=7.1 Hz, 1H), 7.15 (s, 1H), 7.01-6.91 (m, 1H), 5.10 (dd, J=5.2, 13.3 Hz, 1H), 4.52-4.41 (m, 2H), 4.37-4.25 (m, 1H), 3.63 (dd, J=6.1, 10.7 Hz, 1H), 3.30 (s, 3H), 3.26 (br s, 1H), 2.97-2.88 (m, 1H), 2.84 (dd, J=3.3, 6.9 Hz, 1H), 2.63 (br s, 1H), 2.39 (br s, 1H), 2.07-1.95 (m, 1H), 0.91-0.79 (m, 1H). MS (ESI) m/z. 461.2 [M+H]⁺

### Example 85. Synthesis of Compound 311

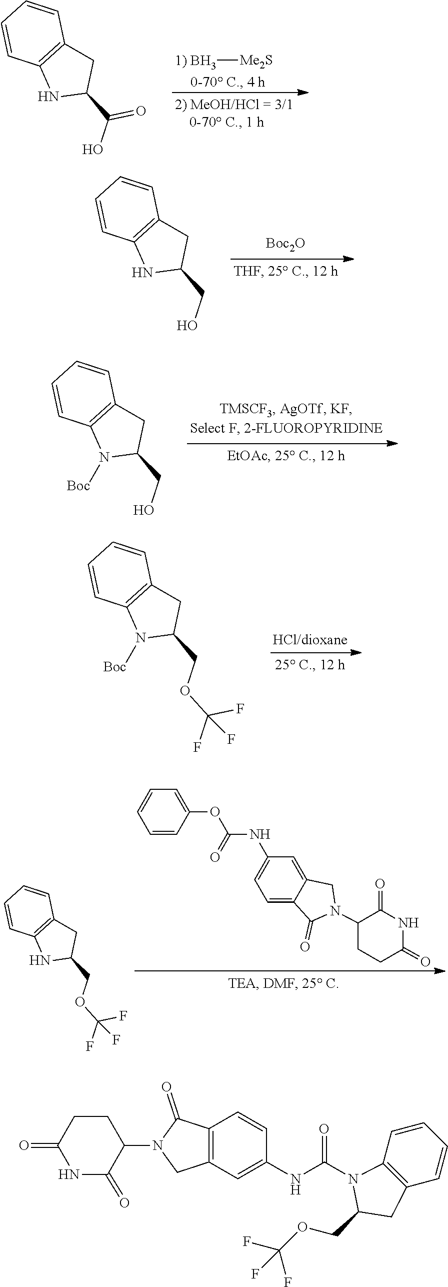

Step 1. To a solution of (S)-indoline-2-carboxylic acid (5.00 g, 30.6 mmol, 1.00 eq) in tetrahydrofuran (50.0 mL) was added borane dimethyl sulfide complex (2.50 M, 24.5 mL, 2.00 eq) at 0° C. under nitrogen atmosphere. Then the reaction mixture was stirred at 70° C. for 4 h. The reaction mixture was quenched by addition methanol (8 mL) at 0° C. Then the reaction mixture was added hydrochloric acid (3 mL) and stirred at 70° C. for 1 h. Then the reaction mixture was concentrated in vacuum. The residue was purified by column chromatography (SiO2, Petroleum ether/Ethyl acetate=1 0/1 to 5/1) to get (S)-indolin-2-ylmethanol (4.20 g, 26.7 mmol, 87% yield, 95% purity) as a yellow solid. ¹H NMR (400 MHz, DMSO-d₆) δ=6.97 (d, J=7.2 Hz, 1H), 6.87 (t, J=7.6 Hz, 1H), 6.51-6.44 (m, 2H), 5.54 (br s, 1H), 4.73 (br t, J=4.8 Hz, 1H), 3.81-3.74 (m, 1H), 3.41 (br dd, J=4.4, 10.0 Hz, 1H), 3.37 (br s, 1H), 2.97 (dd, J=9.2, 16.0 Hz, 1H), 2.63 (dd, J=6.8, 16.0 Hz, 1H).

Step 2. To a solution of (S)-indolin-2-ylmethanol (2.30 g, 15.4 mmol, 1.00 eq) in tetrahydrofuran (23.0 mL) was added di-tert-butyldicarbonate (5.05 g, 23.1 mmol, 5.31 mL, 1.50 eq). Then the reaction mixture was stirred at 25° C. for 12 h. The reaction mixture was diluted with water (200 mL) and extracted with ethyl acetate (3×100 mL). The combined organic layers were washed with brine (3×20 mL), dried over sodium sulfate, filtered and concentrated under reduced pressure to give a residue. The residue was purified by column chromatography (SiO2, Petroleum ether/Ethyl acetate=50/1 to 10/1) and concentrated in vacuum to get tert-butyl (S)-2-(hydroxymethyl)indoline-1-carboxylate (3.60 g, 14.4 mmol, 93% yield, 95% purity) as a yellow solid. ¹H NMR (400 MHz, DMSO-d₆) δ=7.78-7.41 (m, 1H), 7.18 (d, J=7.2 Hz, 1H), 7.11 (t, J=7.6 Hz, 1H), 6.91 (t, J=7.6 Hz, 1H), 4.88 (t, J=5.6 Hz, 1H), 4.34 (dt, J=3.6, 6.4 Hz, 1H), 3.60-3.52 (m, 1H), 3.44-3.34 (m, 1H), 3.21 (br dd, J=9.8, 16.4 Hz, 1H), 2.99 (dd, J=2.1, 16.4 Hz, 1H), 1.51 (s, 9H).

Step 3. To a reaction tube was added silver trifluoromethanesulfonate (1.55 g, 6.02 mmol, 3.00 eq), potassium fluoride (349 mg, 6.02 mmol, 140 μL, 3.00 eq), selectfluor(r)fluorinating reagent (710 mg, 2.01 mmol, 1.00 eq), tert-butyl (2S)-2-(hydroxymethyl)indoline-1-carboxylate (500 mg, 2.01 mmol, 1.00 eq). Then trimethyl(trifluoromethyl)silane (855 mg, 6.02 mmol, 3.00 eq) and 2-fluoropyridine (584 mg, 6.02 mmol, 516 μL, 3.00 eq) in ethyl acetate (10.0 mL) was added in the reaction tube under nitrogen atmosphere. Then the reaction mixture was stirred at 25° C. for 12 h under nitrogen atmosphere. The reaction mixture was filtered. The filtrate was concentrated in vacuum. The residue was purified by reversed phase column chromatography (C18, 80 g; condition: water/acetonitrile=1/0 to 0/1, 0.1% formic acid) and concentrated in vacuum to get tert-butyl (S)-2-((trifluoromethoxy)methyl)indoline-1-carboxylate (200 mg, 560 mol, 27% yield, 89% purity) as yellow oil. MS (ESI) m/z. 262.1 [M+H]⁺

Step 4. To a solution of tert-butyl (2S)-2-(trifluoromethoxymethyl)indoline-1-carboxylate (20.0 mg, 63.0 mol, 1.00 eq) in hydrochloric acid (4 M in dioxane) (5.00 mL) was stirred at 25° C. for 12 h. The reaction mixture was concentrated in vacuum to get (S)-2-((trifluoromethoxy)methyl)indoline (15.0 mg, crude) as yellow oil. MS (ESI) m/z. 218.1 [M+H]⁺

Step 5. To a solution (S)-2-((trifluoromethoxy)methyl)indoline (15.0 mg, 69.0 μmol, 1.00 eq) and phenyl (2-(2,6-dioxopiperidin-3-yl)-1-oxoisoindolin-5-yl)carbamate (26.2 mg, 69.0 mol, 1.00 eq) in N,N-dimethylformamide (1.00 mL) was added triethylamine (20.9 mg, 207 μmol, 28.8 μL, 3.00 eq). Then the reaction mixture was stirred at 50° C. for 1 h. The reaction mixture was adjust pH to 5-6 by formic acid (0.1 mL) and filtered. The filtrate was purified by reversed phase column chromatography (C18, 40 g; condition: water/acetonitrile=1/0 to 0/1, 0.1% formic acid) and lyophilized to afford (2S)—N-(2-(2,6-dioxopiperidin-3-yl)-1-oxoisoindolin-5-yl)-2-((trifluoromethoxy)methyl)indoline-1-carboxamide (13.64 mg, 26.6 μmol, 38% yield, 98% purity) as a white solid. ¹H NMR (400 MHz, DMSO-d₆) δ=10.99 (s, 1H), 9.16 (s, 1H), 8.46 (br d, J=121 Hz, 0.5H), 7.85 (s, 1H), 7.81-7.76 (m, 1H), 7.70-7.64 (m, 1H), 7.62-7.57 (m, 1H), 7.26 (br d, J=6.4 Hz, 1H), 7.17 (br t, J=7.2 Hz, 1H), 7.01-6.94 (m, 1H), 5.22-5.05 (m, 2H), 4.48-4.40 (m, 1H), 4.36-4.27 (m, 1H), 4.27 (br s, 2H), 3.50-3.43 (m, 1H), 3.02-2.95 (m, 1H), 2.94-2.84 (m, 1H), 2.63-2.59 (m, 1H), 2.43-2.36 (m, 1H), 2.06-1.96 (m, 1H). MS (ESI) m/z. 503.3 [M+H]⁺

### Example 86. Synthesis of Compound 315

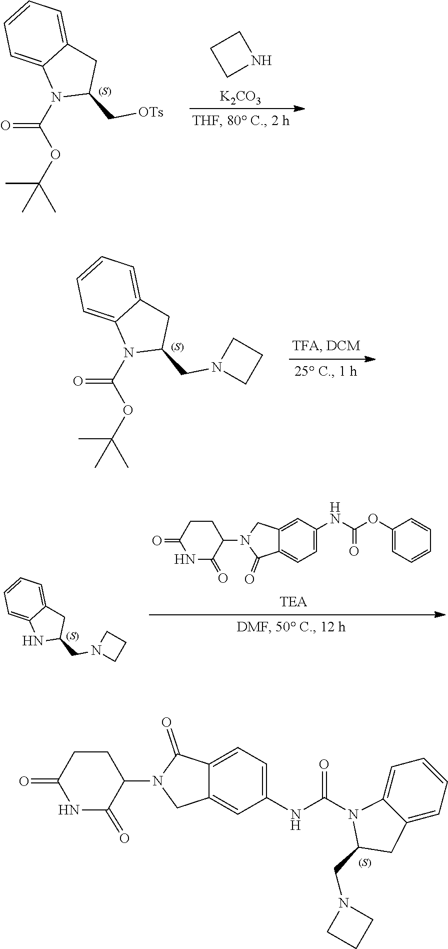

Step 1. To a solution of azetidine (113 mg, 1.98 mmol, 133 μL, 4.00 eq) and tert-butyl (S)-2-((tosyloxy)methyl)indoline-1-carboxylate (200 mg, 495 mol, 1.00 eq) in tetrahydrofuran (2.00 mL) was added potassium carbonate (205 mg, 1.49 mmol, 3.00 eq). The reaction was stirred at 80° C. for 2 h. The reaction was diluted with water (100 mL) and extracted with ethyl acetate (3×100 mL), the organic layers were wash with brine (100 mL), dried over sodium sulfate, filtered and concentrated under reduced pressure to give a residue. The residue was purified by reversed-phase HPLC (0.1% FA condition) to afford tert-butyl (S)-2-(azetidin-1-ylmethyl)indoline-1-carboxylate (25.0 mg, 86.6 mol, 17% yield) as colorless oil. ¹H NMR (400 MHz, CDCl₃) δ=7.87-7.26 (m, 1H), 7.12-6.98 (m, 2H), 6.90-6.80 (m, 1H), 4.39-4.04 (m, 1H), 3.27-3.13 (m, 4H), 2.94 (br d, J=16.0 Hz, 1H), 2.68-2.53 (m, 1H), 2.52-2.41 (m, 1H), 2.12-1.95 (m, 3H), 1.52 (s, 9H).

Step 2. A mixture of tert-butyl (S)-2-(azetidin-1-ylmethyl)indoline-1-carboxylate (25.0 mg, 86.6 mol, 1.00 eq) in trifluoroacetic acid (200 L) and dichloromethane (1.00 mL) was stirred at 25° C. for 1 h. The reaction was concentrated under reduced pressure to afford (S)-2-(azetidin-1-ylmethyl)indoline (16.0 mg, 84.9 mol, 98% yield) as yellow oil. ¹H NMR (400 MHz, CDCl₃) δ=7.09 (td, J=3.6, 7.2 Hz, 2H), 6.92-6.86 (m, 1H), 6.86-6.81 (m, 1H), 4.57-4.39 (m, 2H), 4.30-4.05 (m, 2H), 3.94-3.82 (m, 1H), 3.58-3.46 (m, 1H), 3.35-3.20 (m, 2H), 2.83-2.61 (m, 2H), 2.41-2.23 (m, 1H).

Step 3. To a solution of phenyl (2-(2,6-dioxopiperidin-3-yl)-1-oxoisoindolin-5-yl)carbamate (38.6 mg, 101 mol, 1.20 eq) and (S)-2-(azetidin-1-ylmethyl)indoline (16.0 mg, 84.9 mol, 1.00 eq) in dimethylformamide (1.00 mL) was added triethylamine (25.8 mg, 254 mol, 35.4 μL, 3.00 eq). The reaction was stirred at 50° C. for 12 h. The reaction was filtered to give a residue. The residue was purified by Prep-HPLC (column: Phenomenex luna C18 150*25 mm*10 um; mobile phase: [water (FA)-ACN]; gradient:5%-35% B over 10 min) to afford (2S)-2-(azetidin-1-ylmethyl)-N-(2-(2,6-dioxopiperidin-3-yl)-1-oxoisoindolin-5-yl)indoline-1-carboxamide (18.09 mg, 37.4 mol, 44% yield, 98% purity) as a white solid. ¹H NMR (400 MHz, DMSO-d₆) δ=12.11-11.64 (m, 1H), 10.98 (s, 1H), 7.82 (s, 1H), 7.70 (d, J=8.4 Hz, 1H), 7.68-7.48 (m, 2H), 7.27-7.08 (m, 2H), 7.01-6.86 (m, 1H), 5.10 (dd, J=4.8, 13.4 Hz, 1H), 4.45 (br dd, J=1.6, 17.6 Hz, 2H), 4.35-4.26 (m, 1H), 3.42 (br dd, J=10.4, 16.0 Hz, 3H), 3.35-3.33 (m, 2H), 3.04-2.80 (m, 2H), 2.80-2.67 (m, 2H), 2.62 (br d, J=2.0 Hz, 1H), 2.44-2.38 (m, 1H), 2.23-2.08 (m, 2H), 2.03-1.96 (m, 1H). MS (ESI) m/z 474.4 [M+H]⁺

### Example 87. Synthesis of Compound 323

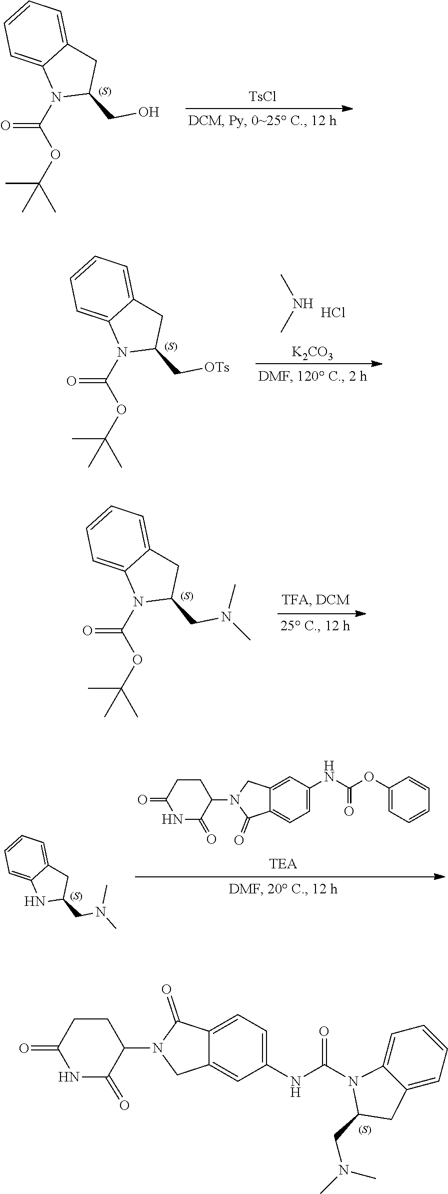

Step 1. To a solution of tert-butyl (S)-2-(hydroxymethyl)indoline-1-carboxylate (5.20 g, 20.8 mmol, 1.00 eq) in pyridine (50.0 mL) and dichloromethane (25.0 mL) was added 4-methylbenzenesulfonyl chloride (7.95 g, 41.7 mmol, 2.00 eq) at 0° C. The reaction was stirred at 25° C. for 12 h. The mixture was concentrated to give a residue. The residue was purified by silica gel chromatography (Petroleum ether/Ethyl acetate=8/1 to 5/1) to afford tert-butyl (S)-2-((tosyloxy)methyl)indoline-1-carboxylate (5.40 g, 13.3 mmol, 64% yield) as a yellow solid. ¹H NMR (400 MHz, DMSO-d₆) δ=7.82-7.41 (m, 5H), 7.28-7.15 (m, 2H), 7.06-6.96 (m, 1H), 4.69-4.56 (m, 1H), 4.29-4.12 (m, 2H), 3.35 (s, 1H), 2.88 (br dd, J=2.3, 16.8 Hz, 1H), 2.47 (s, 3H), 1.47 (br s, 9H). MS (ESI) m/z 426.2 [M+Na]⁺

Step 2. To a solution of tert-butyl (S)-2-((tosyloxy)methyl)indoline-1-carboxylate (500 mg, 1.24 mmol, 1.00 eq), dimethylamine (202 mg, 2.48 mmol, 227 μL, 2.00 eq, hydrochloride) and potassium carbonate (513 mg, 3.72 mmol, 3.00 eq) in dimethyl formamide (5.00 mL) was stirred at 120° C. for 2 h. The reaction mixture was poured into water (100 mL) and extracted with dichloromethane (3×50.0 mL). The combined organic phase was separated, washed with brine (100 mL), dried over anhydrous sodium sulfate, filtered and concentrated to afford tert-butyl (S)-2-((dimethylamino)methyl)indoline-1-carboxylate (300 mg, crude) as yellow oil. MS (ESI) m/z 277.3 [M+H]⁺

Step 3. To a solution of tert-butyl (S)-2-((dimethylamino)methyl)indoline-1-carboxylate (300 mg, 1.09 mmol, 1.00 eq) in dichloromethane (3.00 mL) was added trifluoroacetic acid (3.00 mL). The reaction mixture was stirred at 25° C. for 12 h. The reaction mixture was concentrated to afford (S)-1-(indolin-2-yl)-N,N-dimethylmethanamine (100 mg, crude) as brown oil. MS (ESI) m/z 177.0 [M+H]⁺

Step 4. To a solution of phenyl (2-(2,6-dioxopiperidin-3-yl)-1-oxoisoindolin-5-yl)carbamate (90.0 mg, 237 mol, 1.00 eq), (S)-1-(indolin-2-yl)-N,N-dimethylmethanamine (62.7 mg, 355 mol, 1.50 eq) and triethylamine (72.0 mg, 711 umol, 99.6 uL, 3.00 eq) in dimethyl formamide (1.00 mL) was stirred at 20° C. for 12 h. The reaction mixture was filtered to give a filtrate. The filtrate was purified by Prep-HPLC (column: YMC-Actus Triart C18 150*30 mm*7 um; mobile phase: [water (FA)-ACN]; B %: 5%-35%, 10 min) to afford (2S)-2-((dimethylamino)methyl)-N-(2-(2,6-dioxopiperidin-3-yl)-1-oxoisoindolin-5-yl)indoline-1-carboxamide (30 mg, 60.4 umol, 25% yield, 93% purity, formic acid) as an off-white solid. ¹H NMR (400 MHz, DMSO-d₆) δ=12.17 (s, 1H), 10.98 (br s, 1H), 8.26 (s, 0.5H), 7.75 (br d, J=4.2 Hz, 1H), 7.69 (d, J=7.8 Hz, 1H), 7.64 (d, J=8.2 Hz, 1H), 7.41-7.35 (m, 1H), 7.19 (d, J=7.4 Hz, 1H), 7.14 (t, J=7.8 Hz, 1H), 6.92 (t, J=7.6 Hz, 1H), 5.09 (br dd, J=3.8, 13.2 Hz, 1H), 4.79-4.68 (m, 1H), 4.46-4.38 (m, 1H), 4.33-4.25 (m, 1H), 3.47 (br dd, J=10.2, 16.8 Hz, 2H), 2.96-2.86 (m, 1H), 2.84-2.76 (m, 1H), 2.75-2.68 (m, 1H), 2.63-2.57 (m, 1H), 2.43 (s, 6H), 2.36 (br d, J=4.8 Hz, 1H), 2.04-1.95 (m, 1H). MS (ESI) m/z 462.4 [M+H]⁺

### Example 88. Synthesis of Compound 310

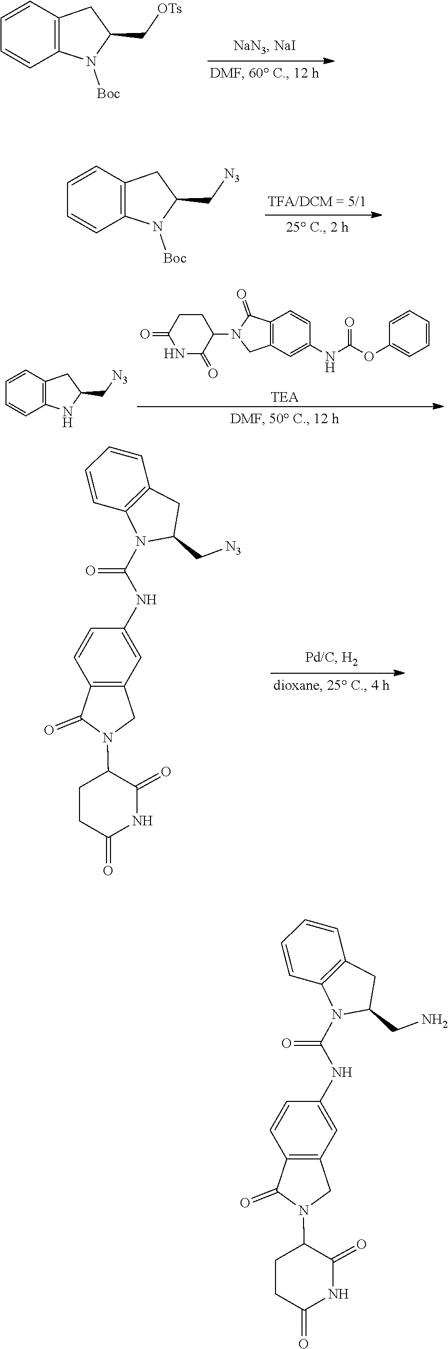

Step 1. To a solution of tert-butyl (S)-2-((tosyloxy)methyl)indoline-1-carboxylate (500 mg, 1.24 mmol, 1.00 eq) in dimethylformamide (5.00 mL) was added sodium azide (510 mg, 7.84 mmol, 6.33 eq) and sodium iodide (18.5 mg, 123 mol, 0.100 eq). The reaction was stirred at 60° C. for 12 h. The reaction was diluted with water (100 mL) and extracted with ethyl acetate (3×50 mL), then the organic layers were washed with brine (50 mL), dried over sodium sulfate, filtered and concentrated under reduced pressure to give a residue. The residue was purified by column chromatography (SiO2, Petroleum ether/Ethyl acetate=10/1 to 5/1) to afford tert-butyl (S)-2-(azidomethyl)indoline-1-carboxylate (300 mg, 1.09 mmol, 88% yield) as colourless oil. ¹H NMR (400 MHz, DMSO-d₆) δ=7.64 (br d, J=1.6 Hz, 1H), 7.32-7.15 (m, 2H), 7.00 (dt, J=0.8, 7.6 Hz, 1H), 4.69-4.51 (m, 1H), 3.73 (dd, J=5.2, 12.4 Hz, 1H), 3.53-3.38 (m, 2H), 2.88 (dd, J=2.6, 16.4 Hz, 1H), 1.58 (s, 9H).

Step 2. A mixture of tert-butyl (S)-2-(azidomethyl)indoline-1-carboxylate (200 mg, 729 mol, 1.00 eq) in trifluoroacetic acid (600 L) and dichloromethane (3.00 mL) was stirred at 25° C. for 2 h. The reaction was concentrated under reduced pressure to afford (S)-2-(azidomethyl)indoline (120 mg, 688 mol, 94% yield) as yellow oil. MS (ESI) m/z 175.1 [M+H]⁺

Step 3. To a solution of phenyl (2-(2,6-dioxopiperidin-3-yl)-1-oxoisoindolin-5-yl)carbamate (261 mg, 688 mol, 1.00 eq) and (S)-2-(azidomethyl)indoline (120 mg, 688 mol, 1.00 eq) in dimethylformamide (5.00 mL) was added triethylamine (209 mg, 2.07 mmol, 287 μL, 3.00 eq). The reaction was stirred at 50° C. for 12 h. The reaction was filtered to give filtrate. The filtrate was purified by reversed-phase HPLC (0.1% FA condition) to afford (2S)-2-(azidomethyl)-N-(2-(2,6-dioxopiperidin-3-yl)-1-oxoisoindolin-5-yl)indoline-1-carboxamide (120 mg, 261 mol, 38% yield) as a green solid. ¹H NMR (400 MHz, DMSO-d₆) δ=10.98 (s, 1H), 9.11 (s, 1H), 7.92-7.76 (m, 2H), 7.72-7.56 (m, 2H), 7.25 (d, J=7.2 Hz, 1H), 7.17 (t, J=7.6 Hz, 1H), 7.03-6.91 (m, 1H), 5.10 (dd, J=5.2, 13.2 Hz, 1H), 5.05-4.97 (m, 1H), 4.51-4.39 (m, 1H), 4.36-4.24 (m, 1H), 3.69-3.61 (m, 1H), 3.58-3.50 (m, 1H), 3.42 (br dd, J=9.2, 16.4 Hz, 1H), 2.98-2.86 (m, 2H), 2.61 (br dd, J=2.8, 15.6 Hz, 1H), 2.40 (dt, J=9.2, 13.2 Hz, 1H), 2.06-1.97 (m, 1H).

Step 4. To a solution of (2S)-2-(azidomethyl)-N-(2-(2,6-dioxopiperidin-3-yl)-1-oxoisoindolin-5-yl)indoline-1-carboxamide (60.0 mg, 130 mol, 1.00 eq) in dioxane (10.0 mL) was added palladium on carbon (10.0 mg, 10% purity). The reaction was stirred at 25° C. for 4 h under hydrogen atmosphere. The reaction was filtered to give filtrate. The filtrate was purified by Prep-HPLC (column: Phenomenex luna C18 150*25 mm*10 um; mobile phase: [water (FA)-ACN]; gradient:5%-35% B over 10 min) to afford (2S)-2-(aminomethyl)-N-(2-(2,6-dioxopiperidin-3-yl)-1-oxoisoindolin-5-yl)indoline-1-carboxamide (17.6 mg, 39.7 mol, 30% yield, 98% purity) as a white solid. ¹H NMR (400 MHz, DMSO-d₆) δ=11.12-10.76 (m, 1H), 8.14 (d, J=2.0 Hz, 0.2H), 7.84 (br d, J=2.6 Hz, 1H), 7.66 (s, 1H), 7.63 (br d, J=2.6 Hz, 1H), 7.59-7.53 (m, 1H), 7.24 (d, J=7.6 Hz, 1H), 7.18 (t, J=7.6 Hz, 1H), 7.01-6.94 (m, 1H), 5.09 (dd, J=5.2, 13.2 Hz, 1H), 4.82-4.63 (m, 1H), 4.49-4.39 (m, 1H), 4.37-4.24 (m, 1H), 3.42 (br d, J=9.6 Hz, 1H), 3.02-2.90 (m, 2H), 2.89-2.78 (m, 2H), 2.60 (br dd, J=2.4, 15.6 Hz, 1H), 2.43-2.32 (m, 1H), 2.07-1.95 (m, 1H). MS (ESI) m/z 434.0 [M+H]⁺

### Example 89. Synthesis of Compound 336

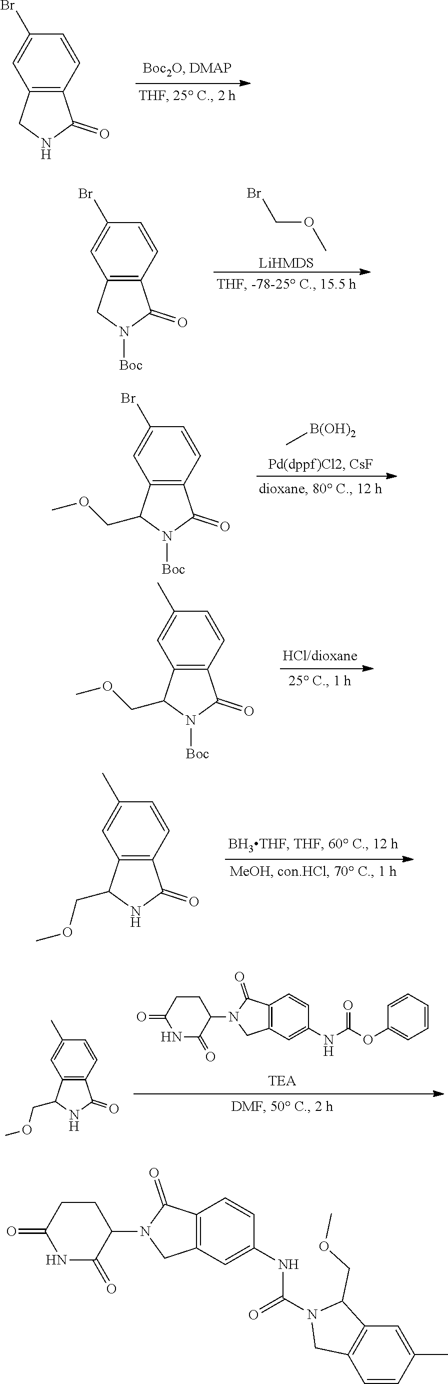

Step 1. To a mixture of 5-bromoisoindolin-1-one (1.00 g, 4.72 mmol, 1.00 eq) in tetrahydrofuran (10.0 mL) was added 4-dimethylaminopyridine (57.6 mg, 472 umol, 0.100 eq) and di-tert-butyldicarbonate (1.54 g, 7.07 mmol, 1.63 mL, 1.50 eq). The mixture was stirred at 25° C. for 2 h. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by column chromatography (SiO2, Petroleum ether/Ethyl acetate=10/1 to 2/1) and concentrated in vacuum to give tert-butyl 5-bromo-1-oxoisoindoline-2-carboxylate (1.20 g, 3.84 mmol, 81% yield) as a white solid. ¹H NMR (400 MHz, DMSO-d₆) δ=7.91 (s, 1H), 7.74-7.67 (m, 2H), 4.77 (s, 2H), 1.52 (s, 9H).

Step 2. To a mixture of tert-butyl 5-bromo-1-oxoisoindoline-2-carboxylate (1.00 g, 3.20 mmol, 1.00 eq) in tetrahydrofuran (10.0 mL) was added lithium bis(trimethylsilyl)amide (1.00 M, 6.41 mL, 2.00 eq) at −78° C. under nitrogen. The mixture was stirred at −78° C. for 1 h. The mixture was added bromo(methoxy)methane (800 mg, 6.41 mmol, 523 μL, 2.00 eq) at −78° C. The mixture was stirred at 0° C. for 2 h under nitrogen. The mixture was stirred at 25° C. for 12 h under nitrogen. The reaction mixture was quenched by addition saturated ammonium chloride (100 mL) aqueous solution, then the mixture was stirred at 0° C. for 0.5 h. The diluted reaction mixture was extracted with ethyl acetate (3×100 mL), washed with brine (100 mL), dried over sodium sulfate, filtered via vacuum filtration, and concentrated in vacuum. The crude product was purified by prep-HPLC (column: Phenomenex luna C18 150*25 mm*10 um; mobile phase: [water (FA)-ACN]; B %: 44%-74%, 10 min) and lyophilized to give tert-butyl 5-bromo-3-(methoxymethyl)-1-oxoisoindoline-2-carboxylate (200 mg, 561 umol, 17% yield) as a white solid. ¹H NMR (400 MHz, DMSO-d₆) δ=7.95 (s, 1H), 7.75-7.71 (m, 1H), 7.69-7.64 (m, 1H), 5.19 (br s, 1H), 4.01-3.93 (m, 1H), 3.91-3.85 (m, 1H), 3.15 (s, 3H), 1.52 (s, 9H).

Step 3. To a mixture of tert-butyl 5-bromo-3-(methoxymethyl)-1-oxoisoindoline-2-carboxylate (140 mg, 393 umol, 1.00 eq) and methylboronic acid (47.1 mg, 786 umol, 2.00 eq) in dioxane (2.00 mL) was added cesium fluoride (209 mg, 1.38 mmol, 50.7 uL, 3.50 eq) and [1,1-bis(diphenylphosphino)ferrocene]dichloropalladium(II) (28.7 mg, 39.3 umol, 0.100 eq). The mixture was stirred at 80° C. for 12 h under nitrogen. The reaction mixture was diluted with water (40 mL) and extracted with ethyl acetate (3×40 mL). The combined organic layers were washed with brine (40 mL), dried over sodium sulfate, filtered and concentrated under reduced pressure to give a residue. The residue was purified by reversed phase (column: spherical C18, 20-45 um, 100 Å, SW 40, mobile phase: [water (0.1% Formic Acid)-ACN) and lyophilized to give tert-butyl 3-(methoxymethyl)-5-methyl-1-oxoisoindoline-2-carboxylate (100 mg, 343 umol, 87% yield) as blue oil. ¹H NMR (400 MHz, DMSO-d₆) δ=7.62 (d, J=7.9 Hz, 1H), 7.47 (s, 1H), 7.34 (d, J=7.8 Hz, 1H), 5.17-5.06 (m, 1H), 3.95-3.90 (m, 1H), 3.88-3.83 (m, 1H), 3.15 (s, 3H), 2.43 (s, 3H), 1.52 (s, 9H).

Step 4. A mixture of tert-butyl 3-(methoxymethyl)-5-methyl-1-oxoisoindoline-2-carboxylate (100 mg, 343 umol, 1.00 eq) in concentrated hydrochloric acid (4.00 M in dioxane) (2.00 mL). The mixture was stirred at 25° C. for 1 h. The mixture was concentrated in vacuum to give 3-(methoxymethyl)-5-methylisoindolin-1-one (90.0 mg, crude) as brown oil. MS (ESI) m/z. 192.1 [M+H]⁺

Step 5. To a solution of 3-(methoxymethyl)-5-methylisoindolin-1-one (90.0 mg, 470 umol, 1.00 eq) in tetrahydrofuran (3.00 mL) was added borane tetrahydrofuran (1.00 M, 1.88 mL, 4.00 eq) at 0° C., the mixture was stirred at 25° C. for 0.5 h and stirred at 60° C. for 11.5 h The reaction mixture was quenched with methanol (0.500 mL). then hydrochloric acid (6 M, 1.00 mL) was added and stirred at 70° C. for 1 h, the reaction was concentrated to give a residue. The residue was purified by reversed phase (0.1% formic acid condition) to afford 1-(methoxymethyl)-6-methylisoindoline (40.0 mg, 225 umol, 47% yield) as colorless oil. MS (ESI) m/z. 178.0 [M+H]⁺

Step 6. To a solution of 1-(methoxymethyl)-6-methylisoindoline (30.0 mg, 169 umol, 1.00 eq), triethylamine (51.4 mg, 508 umol, 70.7 uL, 3.00 eq) in dimethyformamide (2.00 mL) was added phenyl (2-(2,6-dioxopiperidin-3-yl)-1-oxoisoindolin-5-yl)carbamate (64.2 mg, 169 umol, 1.00 eq), the mixture was stirred at 50° C. for 2 h. The reaction mixture was filtered to give a filtrate. The filtrate was purified by prep-HPLC (column: Phenomenex luna C18 150*25 mm*10 um; mobile phase: [water (FA)-ACN]; B %: 30%-60%, 10 min) to afford N-(2-(2,6-dioxopiperidin-3-yl)-1-oxoisoindolin-5-yl)-1-(methoxymethyl)-6-methylisoindoline-2-carboxamide (18.0 mg, 38.9 umol, 22% yield) as a white solid. ¹H NMR (400 MHz, DMSO-d₆) δ=10.97 (s, 1H), 8.86 (br s, 1H), 7.87 (s, 1H), 7.64-7.55 (m, 2H), 7.26-7.19 (m, 2H), 7.14 (br d, J=7.6 Hz, 1H), 5.29 (br s, 1H), 5.08 (dd, J=5.0, 13.1 Hz, 1H), 4.85-4.79 (m, 1H), 4.73-4.64 (m, 1H), 4.45-4.38 (m, 1H), 4.31-4.24 (m, 1H), 3.79-3.67 (m, 2H), 3.27 (s, 3H), 2.90 (br d, J=12.5 Hz, 1H), 2.60 (br d, J=16.9 Hz, 1H), 2.43-2.35 (m, 1H), 2.33 (s, 3H), 2.03-1.95 (m, 1H). MS (ESI) m/z. 463.0 [M+H]⁺

### Example 90. Synthesis of Compound 301

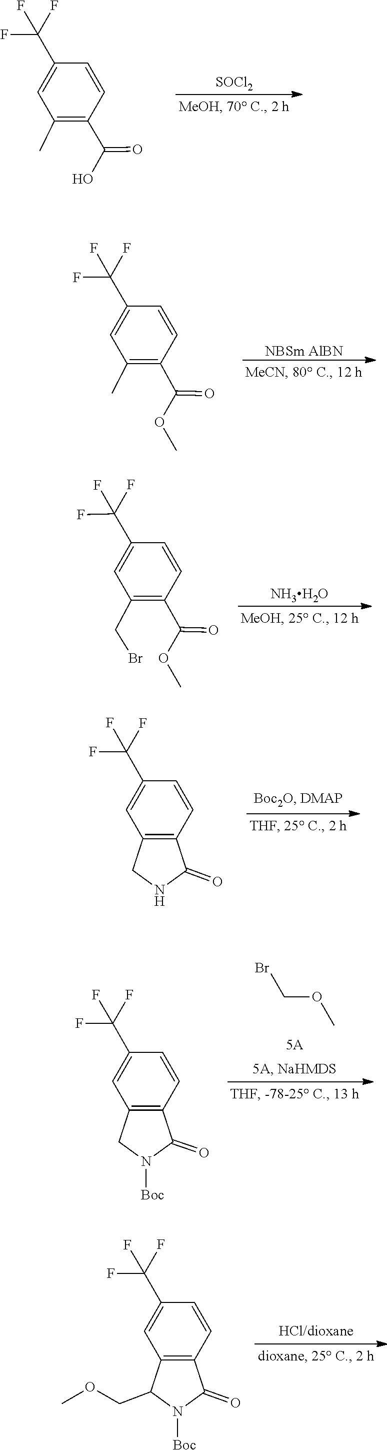

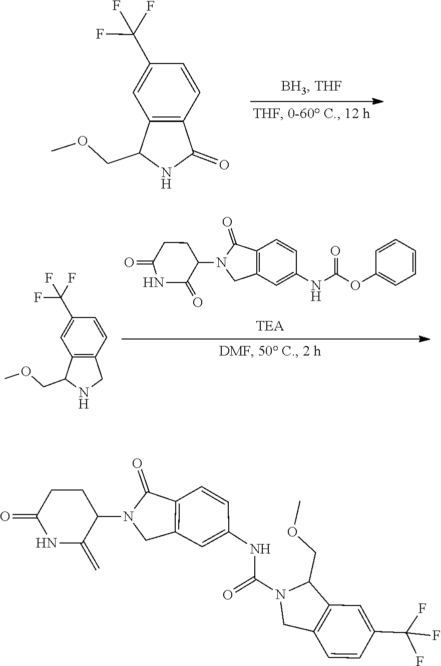 [SMILES: COCC1NC(=O)c2ccc(C(F)(F)F)cc21 ; COCC1NCc2ccc(C(F)(F)F)cc21 ; O=C1CCC(N2Cc3cc(NC(=O)Oc4ccccc4)ccc3C2=O)C(=O)N1 ; C=C1NC(=O)CCC1N1Cc2cc(NC(=O)N3Cc4ccc(C(F)(F)F)cc4C3COC)ccc2C1=O]

Step 1. To a solution of 2-methyl-4-(trifluoromethyl)benzoic acid (4.00 g, 19.6 mmol, 1.00 eq) in methanol (40.0 mL) was added thionyl chloride (12.8 g, 108 mmol, 7.82 mL, 5.50 eq), the mixture was stirred at 70° C. for 2 h. The reaction mixture was diluted with water (30 ml) and exacted with ethyl acetate (3×30 ml). The organic phase was separated, washed with brine (2×10 ml), dried over anhydrous sodium sulfate, filtered and concentrated under reduced pressure to give a residue. The residue was purified by column chromatography (SiO₂, petroleum ether/Ethyl acetate=50/1) to afford methyl 2-methyl-4-(trifluoromethyl)benzoate (4.60 g, crude) as colorless oil. ¹H NMR (400 MHz, DMSO-d₆) δ=8.01-7.95 (m, 1H), 7.74-7.71 (m, 1H), 7.69-7.64 (m, 1H), 3.91-3.86 (m, 3H), 2.60-2.56 (m, 3H). LC-MS (ESI) m/z 218.9 [M+H]⁺

Step 2. To a solution of methyl 2-methyl-4-(trifluoromethyl)benzoate (4.60 g, 21.1 mmol, 1.00 eq) in acetonitrile (50.0 mL) was added N-bromosuccinimide (4.13 g, 23.2 mmol, 1.10 eq) and 2,2-azobisisobutyronitrile (346 mg, 2.11 mmol, 0.100 eq), the mixture was stirred at 80° C. for 12 h. The reaction mixture was diluted with water (30 ml) and exacted with ethyl acetate (3×30 ml). The organic phase was separated, washed with brine (2×10 ml), dried over anhydrous sodium sulfate, filtered and concentrated under reduced pressure to give a residue. The residue was purified by column chromatography (SiO₂, petroleum ether/Ethyl acetate=50/1) to afford methyl 2-(bromomethyl)-4-(trifluoromethyl)benzoate (4.00 g, 13.5 mmol, 64% yield) as colorless oil. ¹H NMR (400 MHz, DMSO-d₆) δ=8.07-8.02 (m, 1H), 7.87-7.81 (m, 1H), 7.75-7.63 (m, 1H), 5.09-5.05 (m, 2H), 3.93-3.90 (m, 3H).

Step 3. To a solution of methyl 2-(bromomethyl)-4-(trifluoromethyl)benzoate (4.00 g, 13.5 mmol, 1.00 eq) in methanol (30.0 mL) was added ammonium hydroxide (10.2 g, 108 mmol, 11.2 mL, 37% purity, 8.00 eq), the mixture was stirred at 25° C. for 12 h. The reaction mixture was diluted with water (30 mL) and exacted with ethyl acetate (3×30 mL). The organic phase was separated, washed with brine (2×10 mL), dried over anhydrous sodium sulfate, filtered and concentrated under reduced pressure to give a residue. The residue was purified by column chromatography (SiO₂, petroleum ether/Ethyl acetate=50/1 to 0/1) to afford 5-(trifluoromethyl) isoindolin-1-one (1.60 g, 7.95 mmol, 59% yield) as a white solid. ¹H NMR (400 MHz, DMSO-d₆) δ=8.94-8.79 (m, 1H), 8.05-7.98 (m, 1H), 7.91-7.81 (m, 2H), 4.51-4.42 (m, 2H). LC-MS (ESI) m/z 202.1 [M+H]⁺

Step 4. To a solution of 5-(trifluoromethyl) isoindolin-1-one (1.60 g, 7.95 mmol, 1.00 eq) in tetrahydrofuran (20.0 mL) was added di-tert-butyl dicarbonate (3.47 g, 15.9 mmol, 3.65 mL, 2.00 eq) and 4-dimethylaminopyridine (486 mg, 3.98 mmol, 0.500 eq) at 25° C., the mixture was stirred at 25° C. for 2 h. The reaction mixture was diluted with water (30 mL) and exacted with ethyl acetate (3×30 mL). The organic phase was separated, washed with brine (2×10 mL), dried over anhydrous sodium sulfate, filtered and concentrated under reduced pressure to give a residue. The residue was purified by column chromatography (SiO₂, petroleum ether/Ethyl acetate=50/1) to afford tert-butyl 1-oxo-5-(trifluoromethyl)isoindoline-2-carboxylate (2.00 g, 6.64 mmol, 83% yield) as a white solid.

¹H NMR (400 MHz, DMSO-d₆) δ=8.12-8.08 (m, 1H), 7.99-7.95 (m, 1H), 7.91-7.87 (m, 1H), 4.92-4.84 (m, 2H), 1.57-1.53 (m, 9H). LC-MS (ESI) m/z 246.0 [M+H−56]⁺

Step 5. To a solution of tert-butyl 1-oxo-5-(trifluoromethyl)isoindoline-2-carboxylate (650 mg, 2.16 mmol, 1.00 eq) in tetrahydrofuran (7.00 mL) was added sodium bis(trimethylsilyl)amide (1.00 M, 2.59 mL, 1.20 eq) at −78° C., the mixture was stirred at −78° C. for 1 h under nitrogen, the mixture was added bromo(methoxy)methane (539 mg, 4.32 mmol, 352 μL, 2.00 eq) in tetrahydrofuran (2.00 mL) at −78° C., the mixture was stirred at 25° C. for 12 h. The reaction mixture was quenched with saturated ammoniumchloride (10 mL) and exacted with ethyl acetate (3×30 mL). The organic phase was separated, washed with brine (2×10 mL), dried over anhydrous sodium sulfate, filtered and concentrated under reduced pressure to give a residue. The residue was purified by column chromatography (SiO₂, petroleum ether/Ethyl acetate=20/1) to afford tert-butyl 3-(methoxymethyl)-1-oxo-5-(trifluoromethyl)isoindoline-2-carboxylate (600 mg, 1.74 mmol, 81% yield) as colorless oil. ¹H NMR (400 MHz, DMSO-d₆) δ=8.25-8.22 (m, 1H), 8.10-8.05 (m, 1H), 8.04-8.01 (m, 1H), 5.00-4.83 (m, 1H), 4.20-4.17 (m, 1H), 4.08-4.02 (m, 1H), 3.30-3.25 (m, 3H), 1.67-1.65 (m, 9H). LC-MS (ESI) m/z 246.0 [M+H−100]⁺

Step 6. To a solution of tert-butyl 3-(methoxymethyl)-1-oxo-5-(trifluoromethyl)isoindoline-2-carboxylate (600 mg, 1.74 mmol, 1.00 eq) in dioxane (4.00 mL) was added hydrochloric acid/dioxane (4.00 M, 6.00 mL, 13.8 eq) at 25° C., the mixture was stirred at 25° C. for 2 h. The reaction mixture was concentrated under reduced pressure to give a residue. The residue was triturated with ethyl acetate (5.00 mL) and filtered. The filter cake was washed with ethyl acetate and dried to afford 3-(methoxymethyl)-5-(trifluoromethyl) isoindolin-1-one (450 mg, crude) as yellow oil. ¹H NMR (400 MHz, DMSO-d₆) δ=9.07-9.03 (m, 1H), 8.04-8.01 (m, 1H), 7.88-7.84 (m, 2H), 4.86-4.82 (m, 1H), 3.73-3.68 (m, 1H), 3.65-3.60 (m, 1H), 3.30-3.27 (m, 3H). LC-MS (ESI) m/z 245.9 [M+H]⁺

Step 7. To a solution of 3-(methoxymethyl)-5-(trifluoromethyl)isoindolin-1-one (450 mg, 1.84 mmol, 1.00 eq) in tetrahydrofuran (5.00 mL) was added borane tetrahydrofuran (1.00 M, 27.5 mL, 15.0 eq) at 0° C., the mixture was stirred at 25° C. for 30 min, then the mixture was stirred at 70° C. for 12 h. The mixture was quenches with methanol (10.0 ml), and added hydrochloric acid at 25° C., the mixture was stirred at 70° C. for 1 h. The mixture was concentrated under reduced pressure to give a residue. The residue was purified by reversed-phase (neutral condition) to give 1-(methoxymethyl)-6-(trifluoromethyl)isoindoline (180 mg, 779 mol, 42% yield) as colorless oil. ¹H NMR (400 MHz, DMSO-d₆) δ=7.87-7.84 (m, 1H), 7.80-7.77 (m, 1H), 7.67-7.63 (m, 1H), 5.15-5.10 (m, 1H), 4.59-4.55 (m, 2H), 4.00-3.95 (m, 1H), 3.83-3.77 (m, 1H), 3.33-3.32 (m, 3H). LC-MS (ESI) m/z 231.9 [M+H]⁺

Step 8. To a solution of 1-(methoxymethyl)-6-(trifluoromethyl)isoindoline (180 mg, 778 mol, 1.00 eq) and triethylamine (315 mg, 3.11 mmol, 433 μL, 4.00 eq) in dimethylformamide (3.00 mL) wad added phenyl (2-(2,6-dioxopiperidin-3-yl)-1-oxoisoindolin-5-yl)carbamate (265 mg, 700 mol, 0.900 eq), the mixture was stirred at 50° C. for 2 h. The reaction mixture was filtered to give a filtrate. The filtrate was purified by Prep-HPLC (column: Phenomenex luna C18 150*25 mm*10 um; mobile phase: [water (FA)-ACN]; gradient:35%-65% B over 10 min) to afford N-(2-(2,6-dioxopiperidin-3-yl)-1-oxoisoindolin-5-yl)-1-(methoxymethyl)-6-(trifluoromethyl)isoindoline-2-carboxamide (177.68 mg, 344 mol, 44% yield) as a white solid. ¹H NMR (400 MHz, DMSO-d₆) δ=10.98 (s, 1H), 8.92 (s, 1H), 7.89 (s, 1H), 7.80 (s, 1H), 7.71 (d, J=8.1 Hz, 1H), 7.62 (q, J=8.3 Hz, 3H), 5.44 (br s, 1H), 5.09 (dd, J=5.1, 13.2 Hz, 1H), 5.02-4.92 (m, 1H), 4.87-4.77 (m, 1H), 4.46-4.37 (m, 1H), 4.35-4.20 (m, 1H), 3.81 (br d, J=3.8 Hz, 2H), 3.26 (s, 3H), 2.97-2.87 (m, 1H), 2.60 (br dd, J=2.0, 15.4 Hz, 1H), 2.39 (br dd, J=4.3, 13.1 Hz, 1H), 2.06-1.94 (m, 1H). LC-MS (ESI) m/z 517.0 [M+H]⁺

### Example 91. Synthesis of Compound 335

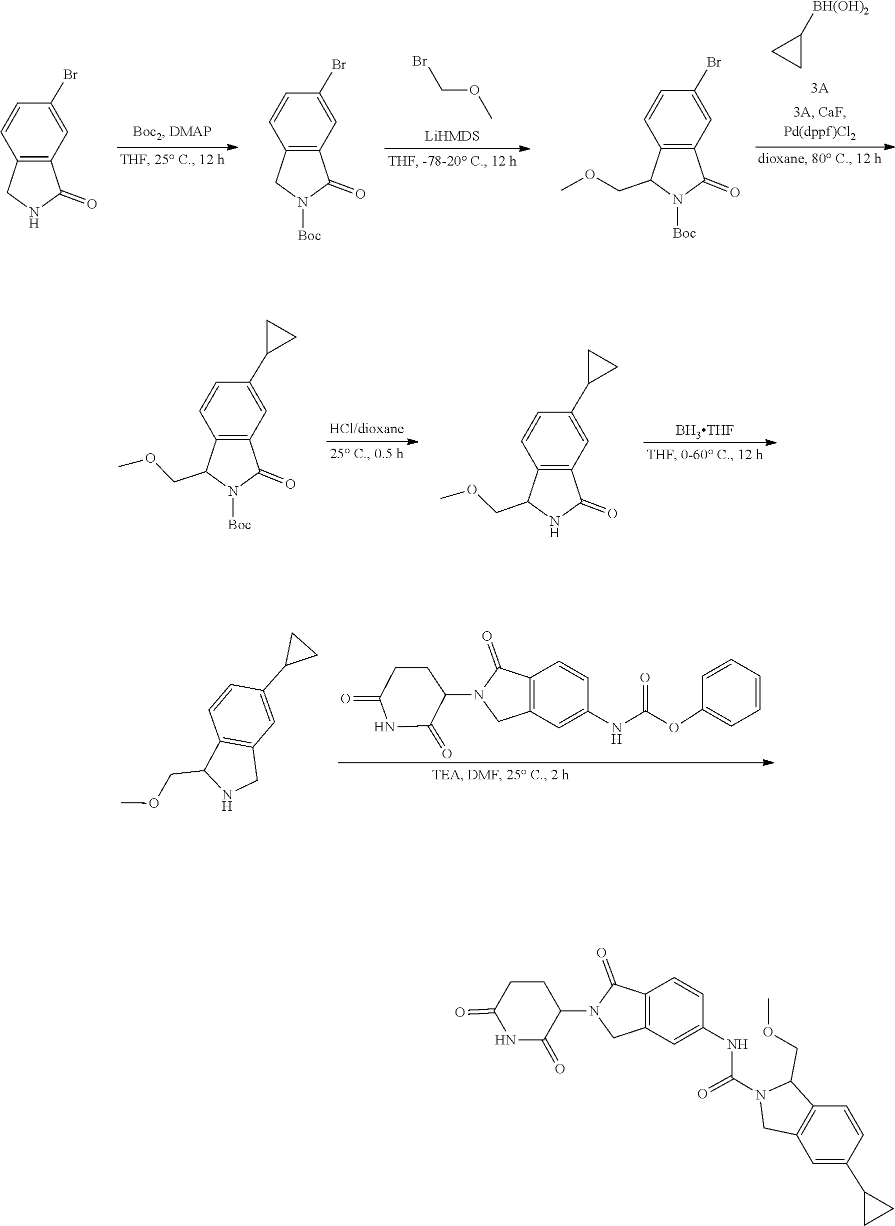

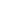

Step 1. To a solution of 6-bromoisoindolin-1-one (3.00 g, 14.1 mmol, 1.00 eq) in tetrahydrofuran (50.0 mL) was added 4-dimethylaminopyridine (172 mg, 1.41 mmol, 0.100 eq) and di-tert-butyldicarbonate (7.72 g, 35.3 mmol, 8.13 mL, 2.50 eq). Then the reaction mixture was stirred at 25° C. for 12 h. The reaction mixture was diluted with water (100 mL) and extracted with ethyl acetate (3×100 mL). The combined organic layers were washed with brine (3×20 mL), dried over sodium sulfate, filtered and concentrated under reduced pressure to give a residue. The residue was purified by column chromatography (SiO₂, Petroleum ether/Ethyl acetate=20/1 to 2/1) and concentrated in vacuum to get tert-butyl 6-bromo-1-oxo-isoindoline-2-carboxylate (4.00 g, 12.6 mmol, 88% yield, 98% purity) as a white solid. ¹H NMR (400 MHz, DMSO-d₆) δ=7.95-7.85 (m, 2H), 7.63 (br d, J=7.6 Hz, 1H), 4.76 (s, 2H), 1.52 (s, 9H).

Step 2. To a solution of tert-butyl 6-bromo-1-oxo-isoindoline-2-carboxylate (3.50 g, 11.2 mmol, 1.00 eq) in tetrahydrofuran (70.0 mL) was added lithium bis(trimethylsilyl)amide (1 M, 13.4 mL, 1.20 eq) dropwise at −78° C. under nitrogen atmosphere. Then the reaction mixture was stirred at −78° C. for 1 h. Then the reaction mixture was added bromo(methoxy)methane (2.80 g, 22.4 mmol, 1.83 mL, 2.00 eq) dropwise at −78° C. under nitrogen atmosphere. Then the reaction mixture was stirred at 25° C. for 12 h under nitrogen atmosphere. The reaction mixture was quenched by addition ammonium chloride (200 mL) at 0° C. and extracted with ethyl acetate (3×150 mL). The combined organic layers were washed with brine (3×20 mL), dried over sodium sulfate, filtered and concentrated under reduced pressure to give a residue. The residue was purified by column chromatography (SiO₂, Petroleum ether/Ethyl acetate=20/1 to 10/1) and concentrated in vacuum to get tert-butyl 5-bromo-1-(methoxymethyl)-3-oxoisoindoline-2-carboxylate (3.00 g, 8.34 mmol, 74% yield, 99% purity) as yellow oil. ¹H NMR (400 MHz, DMSO-d₆) δ=7.95-7.82 (m, 2H), 7.65 (d, J=8.0 Hz, 1H), 5.18 (dd, J=2.4, 4.0 Hz, 1H), 3.97-3.92 (m, 1H), 3.89-3.84 (m, 1H), 3.14 (s, 3H), 1.52 (s, 9H).

Step 3. To a solution of tert-butyl 5-bromo-1-(methoxymethyl)-3-oxo-isoindoline-2-carboxylate (1.00 g, 2.81 mmol, 1.00 eq) and cyclopropylboronic acid (482 mg, 5.61 mmol, 2.00 eq) in dioxane (15.0 mL) was added (1,1′-bis(diphenylphosphino)ferrocene)palladium(II) dichloride (205 mg, 280 umol, 0.100 eq) and cesium fluoride (1.49 g, 9.83 mmol, 362 μL, 3.50 eq) under nitrogen atmosphere. Then the reaction mixture was stirred at 80° C. for 12 h under nitrogen atmosphere. The reaction mixture was diluted with water (100 mL) and extracted with ethyl acetate (3×60 mL). The combined organic layers were washed with brine (3×20 mL), dried over anhydrous sodium sulfate, filtered and concentrated under reduced pressure to give a residue. The residue was purified by column chromatography (SiO₂, Petroleum ether/Ethyl acetate=50/1 to 10/1) and concentrated in vacuum to get tert-butyl 5-cyclopropyl-1-(methoxymethyl)-3-oxo-isoindoline-2-carboxylate (800 mg, 2.34 mmol, 83% yield, 93% purity) as yellow oil. 1H NMR (400 MHz, DMSO-d₆) δ=7.54 (d, J=7.6 Hz, 1H), 7.44-7.41 (m, 2H), 5.13 (dd, J=2.4, 4.0 Hz, 1H), 3.96-3.89 (m, 1H), 3.87-3.79 (m, 1H), 3.15 (s, 3H), 2.10-2.03 (m, 1H), 1.53 (s, 9H), 1.04-0.98 (m, 2H), 0.77-0.72 (m, 2H).

Step 4. To a solution of tert-butyl 5-cyclopropyl-1-(methoxymethyl)-3-oxo-isoindoline-2-carboxylate (800 mg, 2.52 mmol, 1.00 eq) in hydrochloric acid/dioxane (2.00 mL) was stirred at 25° C. for 15 min. The reaction mixture was concentrated in vacuum to get 6-cyclopropyl-3-(methoxymethyl)isoindolin-1-one (660 mg, crude) as yellow oil. MS (ESI) m/z. 218.2 [M+H]⁺

Step 5. To a solution of 6-cyclopropyl-3-(methoxymethyl)isoindolin-1-one (330 mg, 1.52 mmol, 1.00 eq) in tetrahydrofuran (10.0 mL) was added borane-tetrahydrofuran complex (1 M, 10.6 mL, 7.00 eq) dropwise at 0° C. under nitrogen atmosphere. Then the reaction mixture was stirred at 60° C. for 12 h under nitrogen atmosphere. The reaction mixture was quenched by addition methanol (30 mL) at 0° C. and adjusted pH to 1-2 by hydrochloric acid (6 M) and stirred at 80° C. for 1 h. Then the reaction mixture was adjusted pH to 7 by sodium hydroxide (50 mL). Then the reaction mixture was diluted with water (30 mL) and extracted with ethyl acetate (3×20 mL). The combined organic layers were washed with brine (10 mL), dried over sodium sulfate, filtered and concentrated under reduced pressure to give a residue. The residue was purified by reversed phase column chromatography (C18, 80 g; condition: water/acetonitrile=1/0 to 0/1, 0.1% formic acid) and lyophilized to get 5-cyclopropyl-1-(methoxymethyl)isoindoline (100 mg, crude) as yellow oil. MS (ESI) m/z. 204.1 [M+H]⁺

Step 6. To a solution of 5-cyclopropyl-1-(methoxymethyl)isoindoline (50.0 mg, 245 umol, 1.00 eq), phenyl (2-(2,6-dioxopiperidin-3-yl)-1-oxoisoindolin-5-yl)carbamate (74.6 mg, 196 umol, 0.800 eq) in N,N-dimethyl formamide (1.00 mL) was added triethylamine (74.6 mg, 737 umol, 102 uL, 3.00 eq). The mixture was added at 50° C. for 2 h. The mixture was adjusted to pH=5-6 with formic acid and filtered. The filtrate was purified by reversed-phase HPLC (column: spherical C18, 20-45 um, 100 Å, SW 80, mobile phase: [water (0.1% Formic Acid)-acetonitrile]) and lyophilized to afford 5-cyclopropyl-N-(2-(2,6-dioxopiperidin-3-yl)-1-oxoisoindolin-5-yl)-1-(methoxymethyl)isoindoline-2-carboxamide (31.0 mg, 62.8 umol, 26% yield, 99% purity) as a white solid. ¹H NMR (400 MHz, DMSO-d₆) δ=10.97 (s, 1H), 8.87 (br s, 1H), 7.86 (s, 1H), 7.64-7.60 (m, 1H), 7.60-7.54 (m, 1H), 7.26 (d, J=8.0 Hz, 1H), 7.07-7.00 (m, 2H), 5.28 (br s, 1H), 5.08 (dd, J=5.2, 13.2 Hz, 1H), 4.79 (s, 1H), 4.73-4.63 (m, 1H), 4.45-4.37 (m, 1H), 4.32-4.23 (m, 1H), 3.76-3.64 (m, 2H), 3.26 (s, 3H), 2.97-2.85 (m, 1H), 2.61 (br d, J=3.6 Hz, 1H), 2.44-2.35 (m, 1H), 2.03-1.91 (m, 2H), 0.98-0.92 (m, 2H), 0.68 (br dd, J=2.0, 5.2 Hz, 2H). MS (ESI) m/z. 489.2 [M+H]⁺

### Example 92. Synthesis of Compound 333

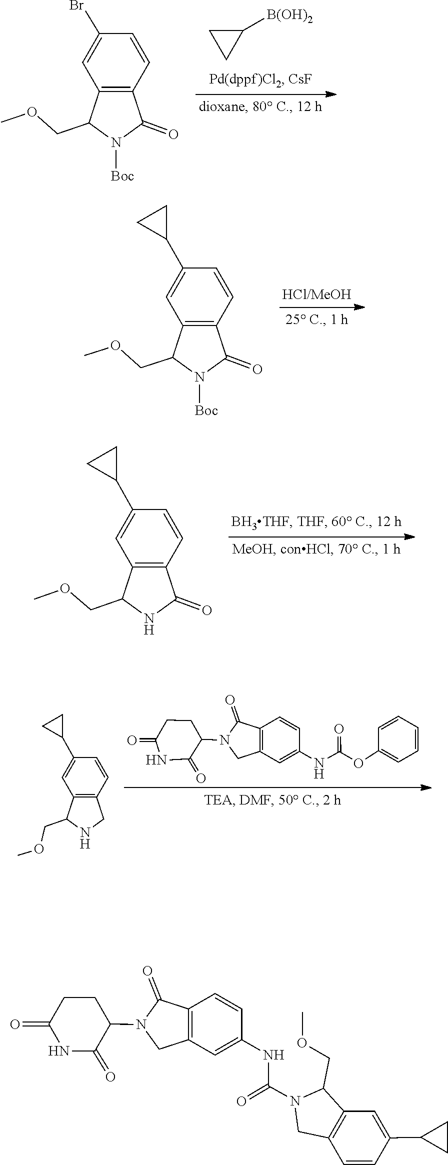

Step 1. To a solution of tert-butyl 5-bromo-3-(methoxymethyl)-1-oxoisoindoline-2-carboxylate (600 mg, 1.68 mmol, 1.00 eq), cyclopropylboronic acid (434 mg, 5.05 mmol, 3.0 eq) and cesium fluoride (895 mg, 5.90 mmol, 217 μL, 3.50 eq) in dioxane (6.00 mL) was added [1,1-bis(diphenylphosphino)ferrocene]dichloropalladium(II) (123 mg, 168 umol, 0.100 eq) at 25° C., the mixture was stirred at 80° C. for 12 h under nitrogen. The reaction mixture was poured into water (80.0 mL) and extracted with ethyl acetate (3×60 mL). The combined organic phase was washed with brine (80 mL), dried over anhydrous sodium sulfate, filtered and concentrated in vacuum to give a residue. The residue was purified by silica gel chromatography (Petroleum ether/Ethyl acetate=1/0 to 1/1) to afford tert-butyl 5-cyclopropyl-3-(methoxymethyl)-1-oxoisoindoline-2-carboxylate (400 mg, 1.26 mmol, 75% yield) as yellow oil. ¹H NMR (400 MHz, DMSO-d₆) δ=7.44 (d, J=8.0 Hz, 1H), 7.23 (s, 1H), 7.06 (br d, J=8.1 Hz, 1H), 4.96 (br s, 1H), 3.83-3.77 (m, 1H), 3.75-3.69 (m, 1H), 3.01 (s, 3H), 1.98-1.89 (m, 1H), 1.38 (s, 9H), 0.93 (dd, J=0.9, 8.3 Hz, 2H), 0.68-0.63 (m, 2H). MS (ESI) m/z 218.1 [M+H−100]⁺

Step 2. A mixture of tert-butyl 5-cyclopropyl-3-(methoxymethyl)-1-oxoisoindoline-2-carboxylate (400 mg, 1.26 mmol, 1.00 eq) in hydrochloric acid in methanol(4 M, 4 mL, 12.7 eq) was stirred at 25° C. for 1 h. The reaction mixture was concentrated to give 5-cyclopropyl-3-(methoxymethyl)isoindolin-1-one (200 mg, crude) as a yellow solid. MS (ESI) m/z 218.0 [M+H]⁺

Step 3. To a solution of 5-cyclopropyl-3-(methoxymethyl)isoindolin-1-one (200 mg, 920 umol, 1.00 eq) in tetrahydrofuran (3.00 mL) was added borane tetrahydrofuran complex (1 M, 3.68 mL, 4.00 eq) at 0° C., the mixture was stirred at 25° C. for 0.5 h then stirred at 70° C. for 11.5 h. The reaction mixture was quenched with methanol (2.00 mL) and added hydrochloric acid (6 M), the mixture was stirred at 80° C. for 1 h, then concentrated to give a residue. The residue was purified by reversed-phase HPLC (0.1% formic acid condition) to afford 6-cyclopropyl-1-(methoxymethyl)isoindoline (100 mg, 49 umol, 53% yield) as a white solid. MS (ESI) m/z 204.0 [M+H]⁺

Step 4. To a solution of 6-cyclopropyl-1-(methoxymethyl)isoindoline (60.0 mg, 295 umol, 1.00 eq), phenyl (2-(2,6-dioxopiperidin-3-yl)-1-oxoisoindolin-5-yl)carbamate (89.6 mg, 236 umol, 0.800 eq) and triethylamine (119 mg, 1.18 mmol, 164 μL, 4.00 eq) in dimethylformamide (2.00 mL) was stirred at 50° C. for 2 h. The reaction mixture was filtered to give a filtrate. The filtrate was purified by Prep-HPLC (column: Phenomenex luna C18 150*25 mm*10 um; mobile phase: [water (FA)-ACN]; B %: 35%-65%, 10 min) to afford a crude product, the crude product was purified by Prep-HPLC (column: Phenomenex C18 150*25 mm*10 um; mobile phase: [water (NH4HCO3)-ACN]; B %: 26%-56%, 14 min) and Prep-HPLC (column: Phenomenex luna C18 150*25 mm*10 um; mobile phase: [water (FA)-ACN]; B %: 40%-60%, 10 min) to afford 6-cyclopropyl-N-(2-(2,6-dioxopiperidin-3-yl)-1-oxoisoindolin-5-yl)-1-(methoxymethyl)isoindoline-2-carboxamide (18.4 mg, 37.66 umol, 13% yield) as a white solid. ¹H NMR (400 MHz, DMSO-d₆) δ=10.96 (s, 1H), 8.87 (br s, 1H), 7.87 (s, 1H), 7.68-7.53 (m, 2H), 7.21 (d, J=7.9 Hz, 1H), 7.13 (s, 1H), 7.03 (d, J=7.9 Hz, 1H), 5.28 (br s, 1H), 5.08 (dd, J=5.1, 13.3 Hz, 1H), 4.82 (br d, J=14.1 Hz, 1H), 4.71-4.63 (m, 1H), 4.49-4.38 (m, 1H), 4.33-4.23 (m, 1H), 3.82-3.77 (m, 1H), 3.71 (br d, J=4.3 Hz, 1H), 3.28 (s, 3H), 2.90 (br d, J=12.8 Hz, 1H), 2.62 (br d, J=2.5 Hz, 1H), 2.39 (br dd, J=4.3, 12.8 Hz, 1H), 2.06-1.85 (m, 2H), 0.96 (dd, J=2.2, 8.2 Hz, 2H), 0.75-0.62 (m, 2H). MS (ESI) m/z 489.1 [M+H]⁺

### Example 93. Synthesis of Compound 317

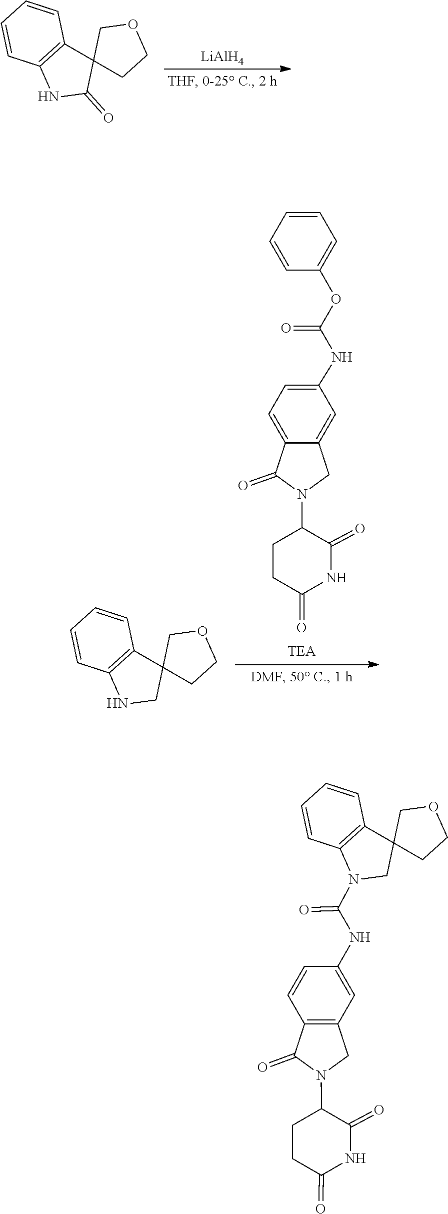 [SMILES: O=C1Nc2ccccc2C12CCOC2 ; "LiAlH4" ; c1ccc2c(c1)NCC21CCOC1 ; O=C1CCC(N2Cc3cc(NC(=O)Oc4ccccc4)ccc3C2=O)C(=O)N1 ; O=C1CCC(N2Cc3cc(NC(=O)N4CC5(CCOC5)c5ccccc54)ccc3C2=O)C(=O)N1]

Step 1. To a solution of 4,5-dihydro-2H-spiro[furan-3,3′-indolin]-2′-one (50.0 mg, 264 mol, 1.00 eq) in tetrahydrofuran (1.00 mL) was added lithium aluminum hydride (20.1 mg, 528 mol, 2.00 eq) at 0° C., the reaction was stirred at 25° C. for 2 h. The reaction mixture was quenched with ammonium chloride (10.0 mL) at 0° C., and extracted with ethyl acetate (3×10.0 mL). The combined organic layers were washed with brine (3×10.0 mL), dried over anhydrous sodium sulfate, filtered and concentrated under reduced pressure to afford 4,5-dihydro-2H-spiro[furan-3,3′-indoline](30.0 mg, 171 mol, 65% yield) as yellow oil. ¹H NMR (400 MHz, DMSO-d₆) δ=7.05 (d, J=7.2 Hz, 1H), 6.94 (br d, J=1.2 Hz, 1H), 6.57 (s, 1H), 6.51 (d, J=7.6 Hz, 1H), 5.56 (br s, 1H), 3.94 (dt, J=4.8, 8.4 Hz, 1H), 3.83 (q, J=7.6 Hz, 1H), 3.71 (d, J=8.4 Hz, 1H), 3.57 (d, J=8.4 Hz, 1H), 3.40-3.35 (m, 2H), 2.14-2.06 (m, 1H), 2.05-1.98 (m, 1H). MS (ESI) m/z 174.1 [M+H]⁺

Step 2. To a solution of 4,5-dihydro-2H-spiro[furan-3,3′-indoline](30.0 mg, 171 mol, 1.00 eq) in dimethylformamide (2.00 mL) was added phenyl (2-(2,6-dioxopiperidin-3-yl)-1-oxoisoindolin-5-yl)carbamate (78.0 mg, 205 mol, 1.20 eq) and triethylamine (52.0 mg, 514 mol, 72.0 μL, 3.00 eq). The reaction was stirred at 50° C. for 1 h. The reaction was filtered to give a residue. The residue was purified by Prep-HPLC (column: Phenomenex luna C18 150*25 mm*10 um; mobile phase: [water (FA)-ACN]; gradient:25%-55% B over 10 min) to afford N-(2-(2,6-dioxopiperidin-3-yl)-1-oxoisoindolin-5-yl)-4,5-dihydro-2H-spiro[furan-3,3′-indoline]-1′-carboxamide (32.82 mg, 69.8 mol, 41% yield, 98% purity) as a yellow solid. ¹H NMR (400 MHz, DMSO-d₆) δ=10.98 (s, 1H), 8.90 (s, 1H), 7.98-7.88 (m, 2H), 7.72-7.59 (m, 2H), 7.31 (d, J=7.6 Hz, 1H), 7.25-7.18 (m, 1H), 7.04-6.95 (m, 1H), 5.09 (dd, J=5.2, 13.2 Hz, 1H), 4.47-4.40 (m, 1H), 4.33-4.26 (m, 1H), 4.23 (d, J=10.4 Hz, 1H), 4.09 (d, J=10.4 Hz, 1H), 4.07-4.02 (m, 1H), 3.93 (q, J=8.0 Hz, 1H), 3.87 (d, J=8.4 Hz, 1H), 3.66 (d, J=8.4 Hz, 1H), 2.98-2.85 (m, 1H), 2.60 (br d, J=17.2 Hz, 1H), 2.39 (br dd, J=4.4, 13.2 Hz, 1H), 2.35-2.24 (m, 2H), 2.24-2.16 (m, 1H). MS (ESI) m/z 461.2 [M+H]⁺

### Example 94. Synthesis of Compound 314

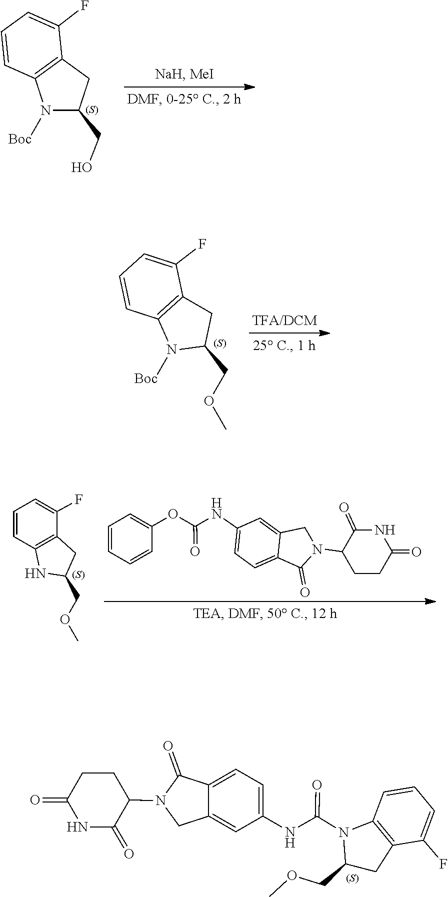

Step 1. To a solution of (S)-tert-butyl 4-fluoro-2-(methoxymethyl)indoline-1-carboxylate (110 mg, 411 mol, 1.00 eq) in dimethylformamide (2.00 mL) was added sodium hydride (32.9 mg, 823 mol, 60% purity, 2.00 eq), the mixture was stirred at 0° C. for 0.5 h, then iodomethane (116 mg, 823 mol, 51.2 μL, 2.00 eq) was added, the mixture was stirred at 25° C. for 1.5 h. The reaction mixture was poured into water (60 mL) and with ethyl acetate (3×30 ml). The organic phase was separated, washed with brine (60 ml), dried over anhydrous sodium sulfate, filtered and concentrated under reduced pressure to give a residue. The residue was purified by silica gel chromatography (Petroleum ether/Ethyl acetate=1/0 to 10/1) to afford (S)-tert-butyl 4-fluoro-2-(methoxymethyl)indoline-1-carboxylate (90.0 mg, 319 mol, 77% yield) as a white solid.

Step 2. To a solution of (S)-tert-butyl 4-fluoro-2-(methoxymethyl)indoline-1-carboxylate (90.0 mg, 319 mol, 1.00 eq) in dichloromethane (2.00 mL) was added trifluoroacetic acid (614 mg, 5.38 mmol, 400 μL, 16.8 eq), the mixture was stirred at 25° C. for 1 h. the reaction mixture was concentrated to give (S)-4-fluoro-2-(methoxymethyl)indoline (50 mg, crude) as yellow oil. MS (ESI) m/z 182.1 [M+H]⁺

Step 3. To a solution of (S)-4-fluoro-2-(methoxymethyl)indoline (50.0 mg, 275 mol, 1.00 eq) and triethylamine (111 mg, 1.10 mmol, 153 μL, 4.00 eq) in dimethylformamide (2.00 mL) was added phenyl (2-(2,6-dioxopiperidin-3-yl)-1-oxoisoindolin-5-yl)carbamate (104 mg, 275 mol, 1.00 eq), the mixture was stirred at 50° C. for 12 h. The reaction mixture was filtered to give a filtrate. The filtrate was purified by Prep-HPLC (column: Phenomenex luna C18 150*25 mm*10 um; mobile phase: [water (FA)-ACN]; gradient:34%-64% B over 10 min) to afford (2S)—N-(2-(2,6-dioxopiperidin-3-yl)-1-oxoisoindolin-5-yl)-4-fluoro-2-(methoxymethyl)indoline-1-carboxamide (13.96 mg, 29.9 mol, 11% yield) as a white solid. ¹H NMR (400 MHz, DMSO-d₆) δ=10.98 (br s, 1H), 9.21 (s, 1H), 7.83 (s, 1H), 7.70-7.51 (m, 3H), 7.28-7.12 (m, 1H), 6.79 (t, J=8.6 Hz, 1H), 5.16-4.99 (m, 2H), 4.50-4.39 (m, 1H), 4.35-4.26 (m, 1H), 3.52 (br d, J=5.5 Hz, 2H), 3.43-3.37 (m, 1H), 3.34 (br s, 3H), 2.91 (br d, J=16.8 Hz, 2H), 2.59 (br s, 1H), 2.41 (br d, J=4.1 Hz, 1H), 2.04-1.99 (m, 1H). MS (ESI) m/z 467.2[M+H]⁺

### Example 95. Synthesis of Compound 303

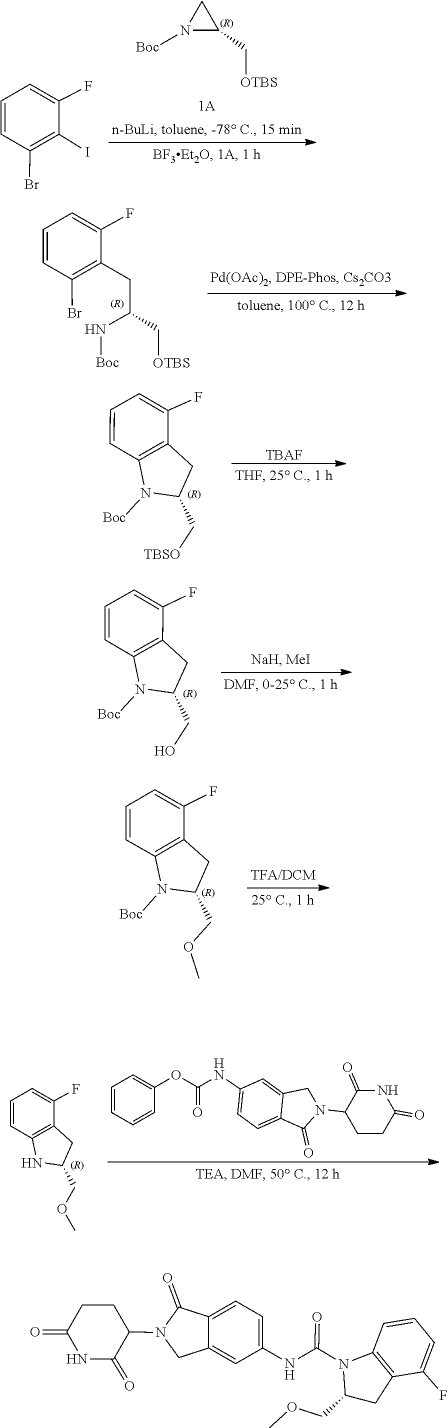

Step 1. To a mixture of 1-bromo-3-fluoro-2-iodobenzene (1.57 g, 5.22 mmol, 1.50 eq) in toluene (15.0 mL) was added n-butyllithium (2.50 M, 1.39 mL, 1.00 eq) at −78° C. The mixture was stirred at −78° C. for 15 min. The mixture was added boron trifluoride diethyl etherat (592 mg, 4.17 mmol, 513 μL, 1.20 eq) and tert-butyl (R)-2-(((tert-butyldimethylsilyl)oxy)methyl)aziridine-1-carboxylate (1.00 g, 3.48 mmol, 1.00 eq) at −78° C. and stirred at −78° C. for 1 h under nitrogen. The reaction mixture was quenched by addition saturated ammonium chloride (30 mL) aqueous solution, then the mixture was stirred at 0° C. for 0.5 h. The diluted reaction mixture was extracted with ethyl acetate (3×100 mL), washed with brine (100 mL), dried over sodium sulfate, filtered via vacuum filtration, and concentrated in vacuum. The crude product was purified by reversed phase (column: spherical C18, 20-45 um, 100 Å, SW 80, mobile phase: [water (0.1% Formic Acid)-ACN) and lyophilized to give tert-butyl (R)-(1-(2-bromo-6-fluorophenyl)-3-((tert-butyldimethylsilyl) oxy)propan-2-yl)carbamate (340 mg, 735 mol, 10% yield) as yellow oil.

¹H NMR (400 MHz, DMSO-d₆) δ=7.45-7.39 (m, 1H), 7.21-7.14 (m, 2H), 6.50 (br d, J=9.3 Hz, 1H), 3.80 (br d, J=5.1 Hz, 1H), 3.55 (br d, J=3.6 Hz, 2H), 2.94 (br dd, J=3.9, 13.1 Hz, 1H), 2.81-2.74 (m, 1H), 1.25 (s, 9H), 0.85 (s, 9H), 0.02 (s, 6H).

Step 2. To a mixture of tert-butyl (R)-(1-(2-bromo-6-fluorophenyl)-3-((tert-butyldimethylsilyl) oxy)propan-2-yl) carbamate (340 mg, 735 mol, 1.00 eq) in toluene (4.00 mL) was added cesium carbonate (479 mg, 1.47 mmol, 2.00 eq), [2-(2-diphenylphosphanylphenoxy)phenyl]-diphenyl-phosphane (79.2 mg, 147 mol, 0.200 eq) and palladium(II) acetate (16.5 mg, 73.5 mol, 0.100 eq). The mixture was stirred at 100° C. for 12 h under nitrogen. The reaction mixture was diluted with water (20 mL) and extracted with ethyl acetate (3×20 mL). The combined organic layers were washed with brine (20 mL), dried over sodium sulfate, filtered and concentrated under reduced pressure to give a residue. The residue was purified by column chromatography (SiO2, Petroleum ether/Ethyl acetate=20/0 to 10/1) and concentrated in vacuum to give tert-butyl (R)-2-(((tert-butyldimethylsilyl)oxy)methyl)-4-fluoroindoline-1-carboxylate (240 mg, 629 mol, 85% yield) as yellow oil. ¹H NMR (400 MHz, DMSO-d₆) δ=7.49-7.24 (m, 1H), 7.19-7.08 (m, 1H), 6.73 (t, J=8.6 Hz, 1H), 4.53-4.37 (m, 1H), 3.79 (br s, 1H), 3.67 (br d, J=9.5 Hz, 1H), 3.27-3.21 (m, 1H), 2.95 (br d, J=16.8 Hz, 1H), 1.51 (s, 9H), 0.67 (s, 9H), −0.03 (s, 3H), −0.12 (s, 3H).

Step 3. To a mixture of tert-butyl (R)-2-(((tert-butyldimethylsilyl)oxy)methyl)-4-fluoroindoline-1-carboxylate (240 mg, 629 mol, 1.00 eq) in tetrahydrofuran (6.00 mL) was added tetrabutylammonium fluoride (1.00 M, 754 μL, 1.20 eq). The mixture was stirred at 25° C. for 1 h. The reaction mixture was diluted with water (20 mL) and extracted with ethyl acetate (3×20 mL). The combined organic layers were washed with brine (20 mL), dried over sodium sulfate, filtered and concentrated under reduced pressure to give tert-butyl (R)-4-fluoro-2-(hydroxymethyl)indoline-1-carboxylate (190 mg, crude) as yellow oil. ¹H NMR (400 MHz, DMSO-d₆) δ=7.40 (br s, 1H), 7.25-7.04 (m, 1H), 6.75 (t, J=8.6 Hz, 1H), 4.92 (t, J=5.7 Hz, 1H), 4.40 (dt, J=3.1, 6.4 Hz, 1H), 3.60-3.50 (m, 1H), 3.48-3.39 (m, 1H), 3.21 (br dd, J=10.1, 16.4 Hz, 1H), 3.01 (dd, J=2.4, 16.4 Hz, 1H), 1.50 (s, 9H).

Step 4. To a mixture of tert-butyl (R)-4-fluoro-2-(hydroxymethyl)indoline-1-carboxylate (90.0 mg, 336 mol, 1.00 eq) in dimethyformamide (1.00 mL) was added sodium hydride (20.2 mg, 505 mol, 60% purity, 1.50 eq) at 0° C. The mixture was added iodomethane (95.6 mg, 673 mol, 41.9 μL, 2.00 eq) at 0° C. and stirred at 25° C. for 1 h. The reaction mixture was quenched by addition saturated ammonium chloride (5 mL) aqueous solution, then the mixture was stirred at 0° C. for 0.5 h. The diluted reaction mixture was extracted with ethyl acetate (3×10 mL), washed with brine (10 mL), dried over sodium sulfate, filtered via vacuum filtration, and concentrated in vacuum. The residue was purified by column chromatography (SiO2, Petroleum ether/Ethyl acetate=1/0 to 10/1) and concentrated in vacuum to give tert-butyl (R)-4-fluoro-2-(methoxymethyl)indoline-1-carboxylate (60 mg, 213 mol, 63% yield) as yellow oil. MS (ESI) m/z. 182.1 [M+H−100]⁺

Step 5. To a solution of tert-butyl (R)-4-fluoro-2-(methoxymethyl)indoline-1-carboxylate (60.0 mg, 213 mol, 1.00 eq) in dichloromethane (1.00 mL) was added trifluoroacetic acid (307 mg, 2.69 mmol, 0.200 mL). The mixture was stirred at 25° C. for 1 h. The mixture was concentrated in vacuum to give (R)-4-fluoro-2-(methoxymethyl)indoline (50.0 mg, 169 mol, 79% yield, trifluoroacetic acid) as yellow oil. MS (ESI) m/z. 182.3 [M+H]⁺

Step 6. To a mixture of (R)-4-fluoro-2-(methoxymethyl)indoline (50.0 mg, 275 mol, 1.00 eq) and phenyl (2-(2,6-dioxopiperidin-3-yl)-1-oxoisoindolin-5-yl)carbamate (104 mg, 275 mol, 1.00 eq) in dimethyformamide (1.00 mL) was added triethylamine (83.7 mg, 827 mol, 115 μL, 3.00 eq). The mixture was stirred at 50° C. for 12 h. The mixture was filtered. The crude product was purified by Prep-HPLC (column: Phenomenex luna C18 150*25 mm*10 um; mobile phase: [water (FA)-ACN]; gradient:33%-63% B over 10 min) and lyophilized to give (2R)—N-(2-(2,6-dioxopiperidin-3-yl)-1-oxoisoindolin-5-yl)-4-fluoro-2-(methoxymethyl)indoline-1-carboxamide (18.05 mg, 38.3 mol, 13% yield, 99% purity) as a white solid. ¹H NMR (400 MHz, DMSO-d₆) δ=10.97 (s, 1H), 9.20 (s, 1H), 7.82 (s, 1H), 7.66 (d, J=8.4 Hz, 1H), 7.60 (d, J=8.0 Hz, 1H), 7.54 (dd, J=1.3, 8.3 Hz, 1H), 7.25-7.12 (m, 1H), 6.78 (t, J=8.6 Hz, 1H), 5.13-4.99 (m, 2H), 4.47-4.38 (m, 1H), 4.36-4.23 (m, 1H), 3.52 (d, J=5.5 Hz, 2H), 3.42-3.35 (m, 1H), 3.34 (s, 3H), 2.97-2.83 (m, 2H), 2.64-2.55 (m, 1H), 2.39 (br dd, J=4.3, 13.1 Hz, 1H), 2.06-1.93 (m, 1H). MS (ESI) m/z. 467.1 [M+H]⁺

### Example 96. Synthesis of Compound 294

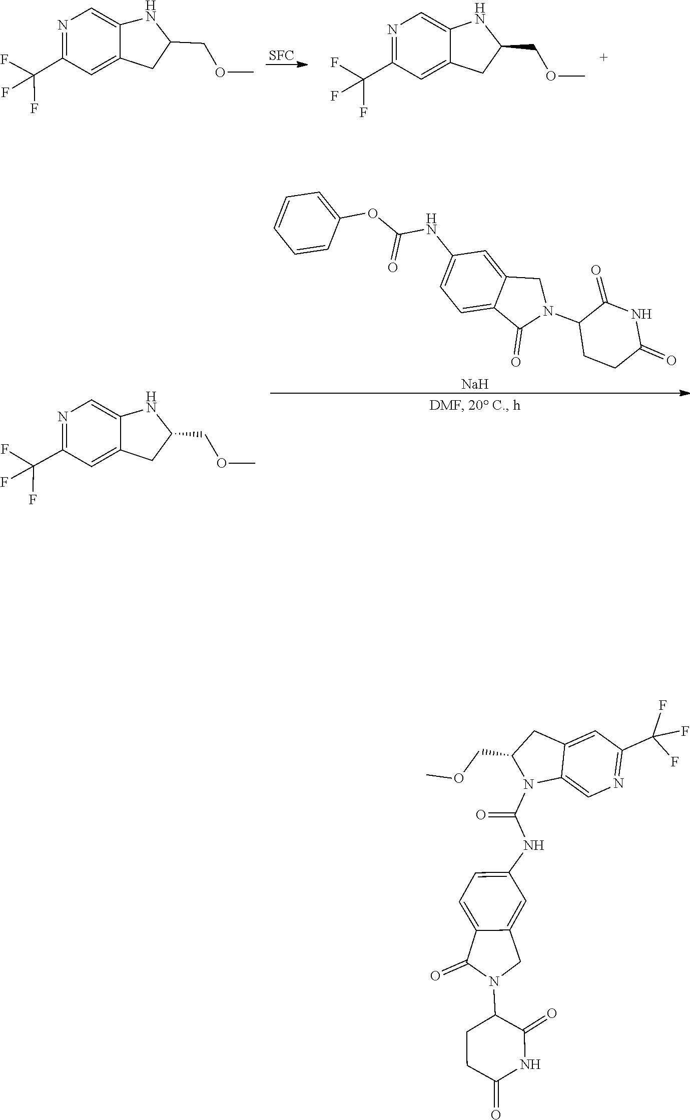

Step 1. The product 2-(methoxymethyl)-5-(trifluoromethyl)-2,3-dihydro-1H-pyrrolo[2,3-c]pyridine (170 mg, 732 mol) was separated by SFC (column: DAICEL CHIRALPAK AD (250 mm*30 mm, 10 um); mobile phase: [CO₂-MeOH (0.1% NH3H2O)]; B %:10%, isocratic elution mode) and concentrated to afford (R)-2-(methoxymethyl)-5-(trifluoromethyl)-2,3-dihydro-1H-pyrrolo[2,3-c]pyridine (85.0 mg, 366 mol, 50% yield) as yellow oil and (S)-2-(methoxymethyl)-5-(trifluoromethyl)-2,3-dihydro-1H-pyrrolo[2,3-c]pyridine (85.0 mg, 366 mol, 50% yield) as yellow oil.

Step 2. To a solution of (S)-2-(methoxymethyl)-5-(trifluoromethyl)-2,3-dihydro-1H-pyrrolo[2,3-c]pyridine (85.0 mg, 366 mol, 1.00 eq), phenyl (2-(2,6-dioxopiperidin-3-yl)-1-oxoisoindolin-5-yl)carbamate (83.3 mg, 219 mol, 0.600 eq) and sodium hydride (43.9 mg, 1.10 mmol, 60% purity, 3.00 eq) in dimethyl formamide (1.50 mL) at 20° C. The reaction mixture was stirred at 20° C. for 1 h. Then phenyl (2-(2,6-dioxopiperidin-3-yl)-1-oxoisoindolin-5-yl)carbamate (83.3 mg, 219 mol, 0.600 eq) and sodium hydride (43.9 mg, 1.10 mmol, 60% purity, 3.00 eq) was added and the mixture was stirred at 20° C. for 1 h. The reaction mixture was added formic acid (3.00 mL) and filtered to give a filtrate. The filtrate was purified by Prep-HPLC (column: Phenomenex luna C18 150*25 mm*10 um; mobile phase: [water (HCl)-ACN]; gradient:23%-53% B over 10 min) and lyophilized to afford (2S)—N-(2-(2,6-dioxopiperidin-3-yl)-1-oxoisoindolin-5-yl)-2-(methoxymethyl)-5-(trifluoromethyl)-2,3-dihydro-1H-pyrrolo[2,3-c]pyridine-1-carboxamide (49.42 mg, 94.5 umol, 25% yield, 99% purity) as a yellow solid. ¹H NMR (400 MHz, DMSO-d₆) δ=11.00 (s, 1H), 9.38 (s, 1H), 9.05 (s, 1H), 7.88 (s, 1H), 7.81 (s, 1H), 7.73-7.66 (m, 1H), 7.64-7.57 (m, 1H), 5.10 (br dd, J=5.1, 13.0 Hz, 2H), 4.51-4.41 (m, 1H), 4.36-4.26 (m, 1H), 3.57-3.50 (m, 3H), 3.30 (s, 3H), 3.05 (br d, J=18.4 Hz, 1H), 2.97-2.85 (m, 1H), 2.64-2.56 (m, 1H), 2.45-2.34 (m, 1H), 2.05-1.95 (m, 1H). MS (ESI) m/z 518.3 [M+H]⁺

### Example 97. Synthesis of Compound 295

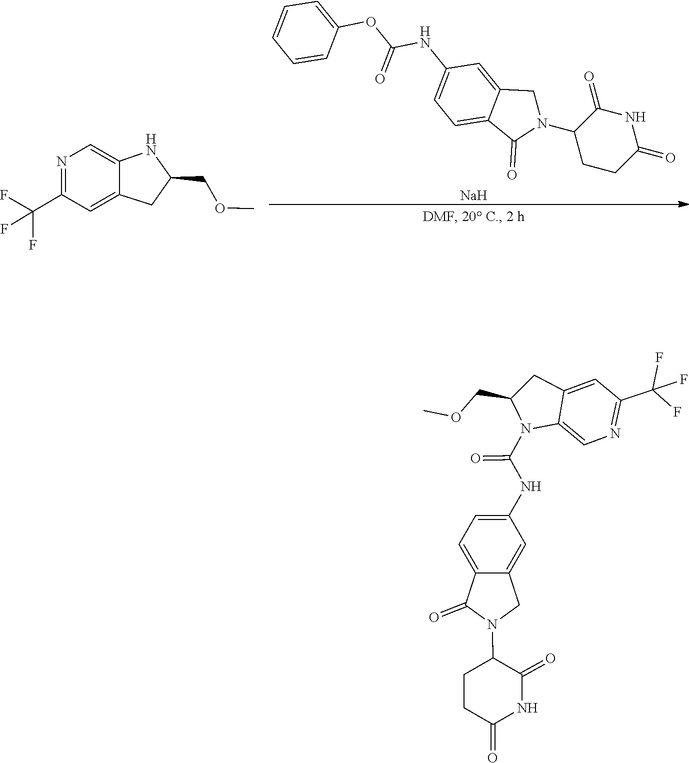

Step 1. To a solution of (R)-2-(methoxymethyl)-5-(trifluoromethyl)-2,3-dihydro-1H-pyrrolo[2,3-c]pyridine (85.0 mg, 366 mol, 1.00 eq), phenyl (2-(2,6-dioxopiperidin-3-yl)-1-oxoisoindolin-5-yl)carbamate (83.3 mg, 219 mol, 0.600 eq) and sodium hydride (43.9 mg, 1.10 mmol, 60% purity, 3.00 eq) in dimethyl formamide (1.50 mL) at 20° C. The reaction mixture was stirred at 20° C. for 1 h. Then phenyl (2-(2,6-dioxopiperidin-3-yl)-1-oxoisoindolin-5-yl)carbamate (83.3 mg, 219 mol, 0.600 eq) and sodium hydride (43.9 mg, 1.10 mmol, 60% purity, 3.00 eq) was added and the mixture was stirred at 20° C. for 1 h. The reaction mixture was added formic acid (3.00 mL) and filtered to give a filtrate. The filtrate was purified by Prep-HPLC (column: Phenomenex luna C18 150*25 mm*10 um; mobile phase: [water (HCl)-ACN]; gradient:23%-53% B over 10 min) and lyophilized to afford (2R)—N-(2-(2,6-dioxopiperidin-3-yl)-1-oxoisoindolin-5-yl)-2-(methoxymethyl)-5-(trifluoromethyl)-2,3-dihydro-1H-pyrrolo[2,3-c]pyridine-1-carboxamide (51.01 mg, 97.5 umol, 26% yield, 99% purity) as a yellow solid. ¹H NMR (400 MHz, DMSO-d₆) δ=11.00 (s, 1H), 9.38 (s, 1H), 9.05 (s, 1H), 7.88 (s, 1H), 7.81 (s, 1H), 7.73-7.66 (m, 1H), 7.64-7.57 (m, 1H), 5.10 (br dd, J=5.1, 13.0 Hz, 2H), 4.51-4.41 (m, 1H), 4.36-4.26 (m, 1H), 3.57-3.50 (m, 3H), 3.30 (s, 3H), 3.05 (br d, J=18.4 Hz, 1H), 2.97-2.85 (m, 1H), 2.64-2.56 (m, 1H), 2.45-2.34 (m, 1H), 2.05-1.95 (m, 1H). MS (ESI) m/z 518.3 [M+H]⁺

### Example 98. Synthesis of Compound 342

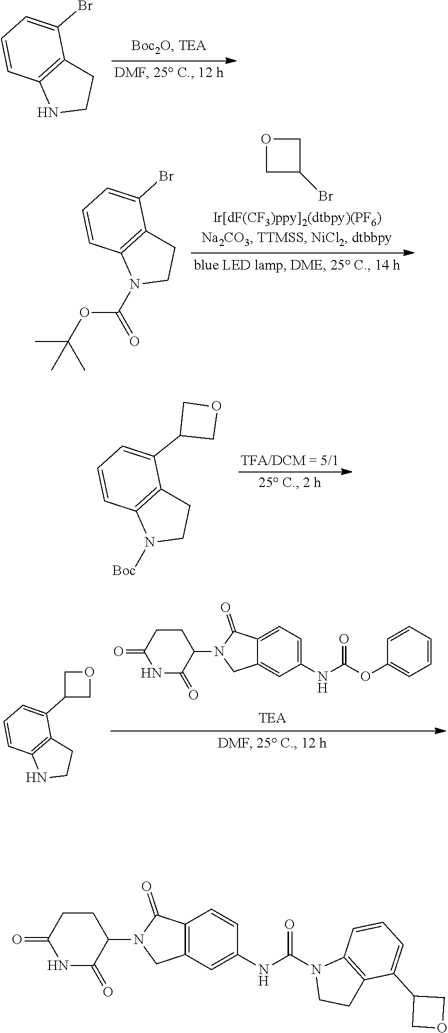

Step 1. To a solution of 4-bromoindoline (3.00 g, 15.1 mmol, 1.00 eq) in dichloromethane (30.0 mL) was added triethylamine (4.60 g, 45.4 mmol, 6.32 mL, 3.00 eq) and di-tert-butyl dicarbonate (4.96 g, 22.7 mmol, 5.22 mL, 1.50 eq) at 25° C., then the mixture was stirred at 25° C. for 16 h. The reaction mixture was concentrated under reduced pressure to give a residue. The residue was purified by chromatography on silica gel (Petroleum ether/Ethyl acetate=50/1) to afford tert-butyl 4-bromoindoline-1-carboxylate (4.50 g, 15.1 mmol, 99% yield) as a colorless oil. ¹H NMR (400 MHz, DMSO-d₆) δ=7.59 (br s, 1H), 7.01 (br d, J=4.1 Hz, 2H), 3.84 (br t, J=8.7 Hz, 2H), 2.90 (br t, J=8.6 Hz, 2H), 1.47-1.41 (m, 9H).

Step 2. To an 15 mL vial equipped with a stir bar was added tert-butyl 4-bromoindoline-1-carboxylate (1.00 g, 3.35 mmol, 1.00 eq), 3-bromooxetane (597 mg, 4.36 mmol, 1.30 eq), 4,4′-Bis(tert-butyl)-2,2′-bipyridine]bis[3,5-difluoro-2-[5-(trifluoromethyl)-2-pyridinyl]phenyl]Iridium(III)hexafluorophosphate (37.6 mg, 33.5 umol, 0.0100 eq), 4,4′-Bis(1,1-dimethylethyl)-2,2′-bipyridine]nickel (II) dichloride (20.02 mg, 50.31 umol, 0.015 eq), bis(trimethylsilyl)silyl-trimethylsilane (833 mg, 3.35 mmol, 1.03 mL, 1.00 eq), sodium carbonate (710 mg, 6.71 mmol, 2.00 eq) in 1,2-dimethoxyethane (20.0 mL). The vial was sealed and placed under nitrogen was added. The reaction was stirred and irradiated with a 10 W blue LED lamp (3 cm away), with cooling water to keep the reaction temperature at 25° C. for 14 hr. The reaction mixture was poured into water (60.0 mL) and extracted with ethyl acetate (3×30.0 mL), the combined organic phase was washed with brine (60.0 mL), dried over anhydrous sodium sulfate, filtered and concentrated under reduced pressure to give a residue. The residue was purified by chromatography on silica gel (Petroleum ether/Ethyl acetate=50/1 to 10/1) to afford tert-butyl 4-(oxetan-3-yl)indoline-1-carboxylate (550 mg, 2.00 mmol, 59% yield) as a yellow solid. ¹H NMR (400 MHz, DMSO-d₆) δ=7.65-7.39 (m, 1H), 7.17 (t, J=7.8 Hz, 1H), 7.04 (d, J=7.8 Hz, 1H), 4.87 (dd, J=5.8, 8.4 Hz, 2H), 4.63 (t, J=6.4 Hz, 2H), 4.25 (br t, J=7.8 Hz, 1H), 3.85 (br t, J=8.6 Hz, 2H), 2.87 (br t, J=8.6 Hz, 2H), 1.47 (s, 9H).

Step 3. To a solution of tert-butyl 4-(oxetan-3-yl)indoline-1-carboxylate (50.0 mg, 181 umol, 1.00 eq) in dichloromethane (1.00 mL) and trifluoroacetic acid (200 uL), then the mixture was stirred at 25° C. for 2 h. The reaction mixture was concentrated under reduced pressure to give 4-(oxetan-3-yl)indoline (30.0 mg, 171 umol, 94% yield) as a brown oil. MS (ESI) m/z 176.2 [M+H]⁺

Step 4. To a solution of 4-(oxetan-3-yl)indoline (30.0 mg, 171 umol, 1.00 eq) in dimethyl formamide (1.00 mL) was added phenyl (2-(2,6-dioxopiperidin-3-yl)-1-oxoisoindolin-5-yl)carbamate (78.0 mg, 205 umol, 1.20 eq) and triethylamine (52.0 mg, 513 umol, 71.5 uL, 3.00 eq), then the mixture was stirred at 25° C. for 12 h. The reaction mixture was filtered to give a crude product. The crude product was purified by Prep-HPLC (column Phenomenex luna C18 150*25 mm*10 um; mobile phase: [water (FA)-ACN]; B %: 17%-47%, 10 min) to afford N-(2-(2,6-dioxopiperidin-3-yl)-1-oxoisoindolin-5-yl)-4-(oxetan-3-yl)indoline-1-carboxamide (21.1 mg, 45.9 umol, 27% yield) as a white solid. ¹H NMR (400 MHz, DMSO-d₆) δ=10.89 (br s, 1H), 8.75 (s, 1H), 7.83 (s, 1H), 7.72 (d, J=8.0 Hz, 1H), 7.56 (s, 2H), 7.13 (t, J=7.9 Hz, 1H), 7.00 (d, J=7.6 Hz, 1H), 5.00 (dd, J=5.1, 13.3 Hz, 1H), 4.83 (dd, J=5.8, 8.5 Hz, 2H), 4.61 (dd, J=6.1, 6.9 Hz, 2H), 4.39-4.31 (m, 1H), 4.27-4.17 (m, 2H), 4.08 (br t, J=8.6 Hz, 2H), 2.97 (br t, J=8.5 Hz, 2H), 2.87-2.78 (m, 1H), 2.53 (br d, J=2.5 Hz, 1H), 2.30 (dt, J=8.9, 13.3 Hz, 1H), 1.96-1.87 (m, 1H). MS (ESI) m/z 461.3 [M+H]⁺

### Example 99. Synthesis of Compound 340

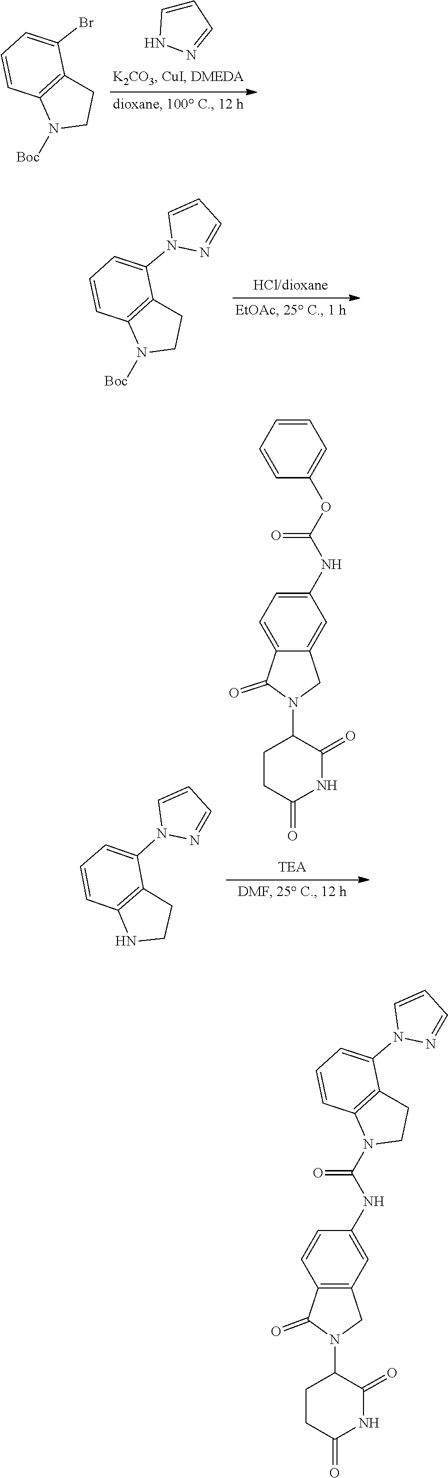 [SMILES: CC(C)(C)OC(=O)N1CCc2c(Br)cccc21 ; c1cn[nH]c1 ; CC(C)(C)OC(=O)N1CCc2c1cccc2-n1cccn1 ; c1cc2c(c(-n3cccn3)c1)CCN2 ; O=C1CCC(N2Cc3cc(NC(=O)Oc4ccccc4)ccc3C2=O)C(=O)N1 ; O=C1CCC(N2Cc3cc(NC(=O)N4CCc5c4cccc5-n4cccn4)ccc3C2=O)C(=O)N1]

Step 1. To a solution of tert-butyl 4-bromoindoline-1-carboxylate (100 mg, 335 umol, 1.00 eq) in dioxane (1.00 mL) was added 1H-pyrazole (27.4 mg, 402 umol, 1.20 eq), copper iodide (12.7 mg, 67.0 umol, 0.200 eq), N¹,N²-dimethylethane-1,2-diamine (14.7 mg, 167 umol, 18.0 uL, 0.500 eq) and potassium carbonate (139 mg, 1.01 mmol, 3.00 eq) under nitrogen atmosphere, then the mixture was stirred at 100° C. for 12 h. The reaction mixture was poured into water (120 mL) and concentrated with ethyl acetate (3×60.0 mL), the combined organic phase was washed with brine (120 mL), dried over anhydrous sodium sulfate, filtered and concentrated in vacuum to give a residue. The residue was purified by chromatography on silica gel (Petroleum ether/Ethyl acetate=50/1 to 20/1) to afford tert-butyl 4-(1H-pyrazol-1-yl)indoline-1-carboxylate (270 mg, 946 umol, 35% yield) as orange oil. MS (ESI) m/z 286.2 [M+H]⁺

Step 2. To a solution of tert-butyl 4-(1H-pyrazol-1-yl)indoline-1-carboxylate (100 mg, 350 umol, 1.00 eq) in ethyl acetate (1.00 mL) was added hydrochloric acid/dioxane (4.00 M, 1.00 mL, 11.4 eq), then the mixture was stirred at 25° C. for 1 h. The reaction mixture was concentrated in vacuum to give a residue. The product 4-(1H-pyrazol-1-yl)indoline (60.0 mg, crude) was obtained as orange solid. MS (ESI) m/z 186.1 [M+H]⁺

Step 3. To a solution of 4-(1H-pyrazol-1-yl)indoline (60.0 mg, 323 umol, 1.00 eq) in dimethylformamide (1.00 mL) was added phenyl (2-(2,6-dioxopiperidin-3-yl)-1-oxoisoindolin-5-yl)carbamate (86.0 mg, 226 umol, 0.700 eq) and triethylamine (98.3 mg, 971 umol, 135 μL, 3.00 eq), then the mixture was stirred at 25° C. for 12 h. The reaction mixture was filtered to give a crude product. The crude product was purified by prep-HPLC (column: Phenomenex Luna C18 150*25 mm*10 um; mobile phase: [water (FA)-ACN]; B %: 23%-53%, 10 min) to afford N-(2-(2,6-dioxopiperidin-3-yl)-1-oxoisoindolin-5-yl)-4-(1H-pyrazol-1-yl)indoline-1-carboxamide (19.51 mg, 42.5 umol, 12.8% yield) as a white solid. ¹H NMR (400 MHz, DMSO-d₆) δ=10.97 (br d, J=2.0 Hz, 1H), 8.93 (s, 1H), 8.30 (d, J=2.0 Hz, 1H), 7.98-7.88 (m, 2H), 7.77 (d, J=1.2 Hz, 1H), 7.71-7.61 (m, 2H), 7.36-7.27 (m, 1H), 7.25-7.18 (m, 1H), 6.55 (s, 1H), 5.10 (dd, J=5.2, 13.2 Hz, 1H), 4.50-4.41 (m, 1H), 4.36-4.27 (m, 1H), 4.23 (br t, J=8.8 Hz, 2H), 3.45 (br t, J=8.8 Hz, 2H), 2.99-2.87 (m, 1H), 2.61 (br d, J=17.2 Hz, 1H), 2.40 (br dd, J=4.4, 13.2 Hz, 1H), 2.05-1.96 (m, 1H). MS (ESI) m/z 471.1 [M+H]⁺

### Example 100. Synthesis of Compound 339

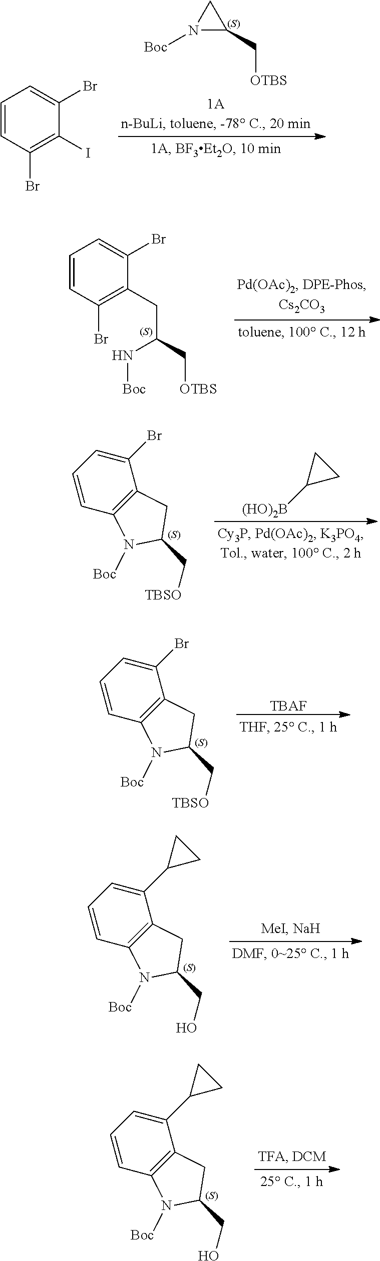

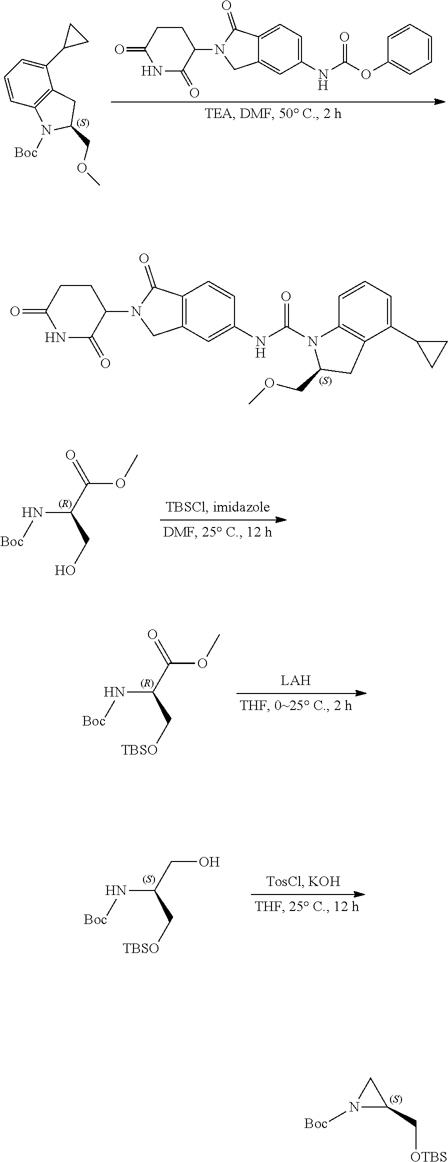

Step 1. To a solution of methyl (tert-butoxycarbonyl)-D-serinate (6.00 g, 27.3 mmol, 1.00 eq) in dimethylformamide (60.0 mL) was added imidazole (5.59 g, 82.1 mmol, 3.00 eq) and tert-butyldimethylsilyl chloride (4.95 g, 32.8 mmol, 4.02 mL, 1.20 eq) at 0° C., the mixture was stirred at 25° C. for 12 h. The reaction mixture was diluted with water (50.0 ml) and exacted with ethyl acetate (3×100 mL). The organic phase was separated, washed with brine (100 ml), dried over anhydrous sodium sulfate, filtered and concentrated under reduced pressure to give a residue. The residue was purified by column chromatography (SiO, petroleum ether/ethyl acetate=50/1 to 20/1) to afford methyl N-(tert-butoxycarbonyl)-O-(tert-butyldimethylsilyl)-D-serinate (5.00 g, 15.0 mmol, 55% yield) as colorless oil. ¹H NMR (400 MHz, DMSO-d₆) δ=6.85 (br d, J=6.6 Hz, 1H), 4.21-4.08 (m, 1H), 3.77 (d, J=5.6 Hz, 2H), 3.61 (s, 3H), 1.36 (s, 9H), 0.82-0.81 (m, 9H), 0.00 (s, 6H).

Step 2. To a solution of methyl N-(tert-butoxycarbonyl)-O-(tert-butyldimethylsilyl)-D-serinate (5.00 g, 15.0 mmol, 1.00 eq) in tetrahydrofuran (50.0 mL) was added lithium aluminum hydride (853 mg, 22.5 mmol, 1.50 eq) at 0° C., the mixture was stirred at 25° C. for 2 h. The reaction mixture was added sodium sulfate decahydrate (10.0 g) and filtered to give a filtrate. The filtrate was concentrated to give a residue. The residue was purified by silica gel chromatography (SiO, petroleum ether/ethyl acetate=1/0 to 20/1) to afford tert-butyl (S)-(1-((tert-butyldimethylsilyl)oxy)-3-hydroxypropan-2-yl)carbamate (2.50 g, 8.18 mmol, 54% yield) as colorless oil. ¹H NMR (400 MHz, DMSO-d₆) δ=6.32 (br d, J=7.6 Hz, 1H), 4.54 (t, J=5.5 Hz, 1H), 3.62-3.54 (m, 1H), 3.51-3.43 (m, 2H), 1.38 (s, 9H), 0.86 (s, 9H), 0.03 (s, 6H).

Step 3. Potassium hydroxide (918 mg, 16.3 mmol, 5.00 eq) was added to a solution of tert-butyl (S)-(1-((tert-butyldimethylsilyl)oxy)-3-hydroxypropan-2-yl)carbamate (1.00 g, 3.27 mmol, 1.00 eq) and toluenesulfonyl (624 mg, 3.27 mmol, 1.00 eq) in tetrahydrofuran (4.00 mL), the mixture was stirred at 25° C. for 12 h. The reaction mixture was concentrated to give a residue. The residue was purified by silica gel chromatography (petroleum ether/ethyl acetate=1/0 to 50/1) to afford tert-butyl (S)-(1-((tert-butyldimethylsilyl)oxy)-3-hydroxypropan-2-yl)carbamate (500 mg, 1.74 mmol, 53% yield) as colorless oil. ¹H NMR (400 MHz, CDCl₃) δ=3.72 (dd, J=4.8, 11.4 Hz, 1H), 3.55 (dd, J=4.9, 11.4 Hz, 1H), 2.53-2.45 (m, 1H), 2.18 (d, J=6.0 Hz, 1H), 1.99 (d, J=3.8 Hz, 1H), 1.37 (s, 9H), 0.82 (s, 9H), 0.00 (d, J=3.0 Hz, 6H).

Step 4. To a solution of 1,3-dibromo-2-iodo-benzene (580 mg, 1.60 mmol, 1.00 eq) in toluene (20.0 mL) was added n-butyllithium (2.5 M, 768 μL, 1.20 eq) at −78° C., the mixture was stirred at −78° C. for 20 min, then tert-butyl (S)-(1-((tert-butyldimethylsilyl)oxy)-3-hydroxypropan-2-yl)carbamate (460 mg, 1.60 mmol, 1.00 eq) in toluene (5.00 mL) was added dropwise, then boron trifluoride diethyl etherate (272 mg, 1.92 mmol, 236 μL, 1.20 eq) was added, the mixture was stirred for at −78° C. 10 min. The reaction mixture was warmed to 25° C. and quenched with saturated sodium bicarbonate (20 mL), then poured into water (60 mL) and extracted with ethyl acetate (3×30 mL). The combined organic phase was washed with brine (100 mL), dried over anhydrous sodium sulfate, filtered and concentrated in vacuum to give a residue. The residue was purified by reversed-phase HPLC (0.1% FA condition) to afford tert-butyl (S)-(1-((tert-butyldimethylsilyl)oxy)-3-(2,6-dibromophenyl)propan-2-yl)carbamate (250 mg, 477 umol, 30% yield) as colorless oil. ¹H NMR (400 MHz, CDCl₃) δ=7.40 (br d, J=8.0 Hz, 2H), 6.86-6.78 (m, 1H), 4.91-4.74 (m, 1H), 4.22-4.05 (m, 1H), 3.63 (br d, J=3.0 Hz, 2H), 3.36-3.25 (m, 1H), 3.07-2.89 (m, 1H), 1.17 (br s, 9H), 0.84 (d, J=0.8 Hz, 9H), 0.00 (d, J=1.9 Hz, 6H). (The spectra comes from pilot run). MS (ESI) m/z 424.1 [M+H+2-100]⁺

Step 5. To a solution of tert-butyl (S)-(1-((tert-butyldimethylsilyl)oxy)-3-(2,6-dibromophenyl)propan-2-yl)carbamate (230 mg, 439 umol, 1.00 eq), [2-(2-diphenylphosphanylphenoxy)phenyl]-diphenyl-phosphane (47.3 mg, 87.9 umol, 0.200 eq) and cesium carbonate (429 mg, 1.32 mmol, 3.00 eq) in toluene (10.0 mL) was added palladium acetate (9.87 mg, 43.9 umol, 0.100 eq), the mixture was stirred at 100° C. for 12 h under nitrogen atmosphere. The reaction mixture was concentrated to give a residue. The residue was purified by silica gel chromatography (petroleum ether/ethyl acetate=1/0 to 50/1) to afford tert-butyl (S)-4-bromo-2-(((tert-butyldimethylsilyl)oxy)methyl)indoline-1-carboxylate (130 mg, 293 umol, 66% yield) as colorless oil. MS (ESI) m/z 342.1.1 [M+H−100]⁺

Step 6. To a solution of tert-butyl (S)-4-bromo-2-(((tert-butyldimethylsilyl)oxy)methyl)indoline-1-carboxylate (110 mg, 248 umol, 1.00 eq), cyclopropylboronic acid (85.4 mg, 994 umol, 4.00 eq) and potassium carbonate (158 mg, 745 umol, 3.00 eq) in toluene (5.00 mL) and water (0.500 mL) was added tricyclohexylphosphane (13.9 mg, 49.7 umol, 16.1 uL, 0.200 eq) and palladium acetate (5.58 mg, 24.8 umol, 0.100 eq), the mixture was stirred at 100° C. for 12 h under nitrogen atmosphere. The reaction mixture was poured into water (80.0 mL) and extracted with ethyl acetate (3×30 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=1/0 to 20/1) to afford tert-butyl (S)-2-(((tert-butyldimethylsilyl)oxy)methyl)-4-cyclopropylindoline-1-carboxylate (90.0 mg, 222 umol, 89% yield) as colorless oil. MS (ESI) m/z 304.2 [M+H−100]⁺

Step 7. To a solution of tert-butyl (S)-2-(((tert-butyldimethylsilyl)oxy)methyl)-4-cyclopropylindoline-1-carboxylate (90.0 mg, 222 umol, 1.00 eq) in tetrahydrofuran (2.00 mL) was added tetrabutylammonium fluoride (1 M, 267 μL, 1.20 eq), the mixture was stirred at 25° C. for 1 h. The reaction mixture was poured into water (80.0 mL) and extracted with ethyl acetate (3×40 mL). The combined organic phase was washed with brine (100 mL), dried over anhydrous sodium sulfate, filtered and concentrated in vacuum to give tert-butyl (S)-4-cyclopropyl-2-(hydroxymethyl)indoline-1-carboxylate (60.0 mg, 207 umol, 92% yield) as colorless oil. MS (ESI) m/z 312.1 [M+23]⁺

Step 8. To a solution of tert-butyl (S)-4-cyclopropyl-2-(hydroxymethyl)indoline-1-carboxylate (30.0 mg, 103 umol, 1.00 eq) in dimethylformamide (1.00 mL) was added sodium hydride (6.22 mg, 155 umol, 60% purity, 1.50 eq), the mixture was stirred at 0° C. for 10 min, then iodomethane (17.6 mg, 124 umol, 7.74 uL, 1.20 eq) was added, the mixture was stirred at 25° C. for another 50 min. The reaction mixture was poured into water (30.0 mL) and extracted with ethyl acetate (3×15 mL). The combined organic phase was washed with brine (60 mL), dried over anhydrous sodium sulfate, filtered and concentrated in vacuum to give a residue. The residue was purified by silica gel chromatography (petroleum ether/ethyl acetate=1/0 to 10/1) to afford tert-butyl (S)-4-cyclopropyl-2-(methoxymethyl)indoline-1-carboxylate (40.0 mg, 131 umol, 63% yield) as colorless oil. ¹H NMR (400 MHz, DMSO-d₆) δ=7.45-7.31 (m, 1H), 7.03 (t, J=8.0 Hz, 1H), 6.46 (d, J=7.8 Hz, 1H), 4.61-4.51 (m, 1H), 3.51 (br d, J=3.3 Hz, 2H), 3.27 (s, 3H), 2.98 (br dd, J=2.4, 16.8 Hz, 1H), 1.85-1.77 (m, 1H), 1.51 (s, 9H), 0.92 (br d, J=8.4 Hz, 2H), 0.69-0.59 (m, 2H). MS (ESI) m/z 204.1 [M+H−100]⁺

Step 9. To a solution of tert-butyl (S)-4-cyclopropyl-2-(methoxymethyl)indoline-1-carboxylate (40.0 mg, 131 umol, 1.00 eq) in dichloromethane (2.00 mL) was added trifluoroacetic acid (616 mg, 5.40 mmol, 400 μL, 40.9 eq), the mixture was stirred at 25° C. for 1 h. The reaction mixture was concentrated to give (S)-4-cyclopropyl-2-(methoxymethyl)indoline (25.0 mg, 122 umol, 93% yield) was obtained as a yellow oil. MS (ESI) m/z 204.1 [M+H]⁺

Step. 10. To a solution of (S)-4-cyclopropyl-2-(methoxymethyl)indoline (25.0 mg, 122 umol, 1.00 eq) and triethylamine (49.8 mg, 491 umol, 68.0 uL, 4.00 eq) in dimethylformamide (2.00 mL) was added phenyl (2-(2,6-dioxopiperidin-3-yl)-1-oxoisoindolin-5-yl)carbamate (42.0 mg, 110 umol, 0.900 eq), the mixture was stirred at 50° C. for 2 h. The reaction mixture was filtered to give a filtrate. The filtrate was purified by Prep-HPLC (column: Phenomenex luna C18 150*25 mm*10 um; mobile phase: [water (FA)-ACN]; B %: 39%-69%, 10 min) and lyophilized to afford (2S)-4-cyclopropyl-N-(2-(2,6-dioxopiperidin-3-yl)-1-oxoisoindolin-5-yl)-2-(methoxymethyl)indoline-1-carboxamide (12.69 mg, 25.98 umol, 21% yield) as a yellow solid. ¹H NMR (400 MHz, DMSO-d₆) δ=10.98 (br s, 1H), 9.17 (s, 1H), 7.82 (s, 1H), 7.72-7.63 (m, 1H), 7.59-7.45 (m, 2H), 7.12-6.97 (m, 1H), 6.54-6.45 (m, 1H), 5.10 (br dd, J=4.4, 13.6 Hz, 1H), 5.02-4.93 (m, 1H), 4.49-4.40 (m, 1H), 4.36-4.26 (m, 1H), 3.51 (br s, 2H), 3.37 (br s, 3H), 3.30-3.28 (m, 1H), 2.94 (br d, J=15.0 Hz, 2H), 2.65-2.61 (m, 1H), 2.43-2.37 (m, 1H), 2.06-1.97 (m, 1H), 1.90-1.79 (m, 1H), 0.94 (br d, J=7.8 Hz, 2H), 0.66 (br dd, J=4.6, 10.1 Hz, 2H). MS (ESI) m/z 489.2 [M+H]⁺

### Example 101. Synthesis of Compound 332

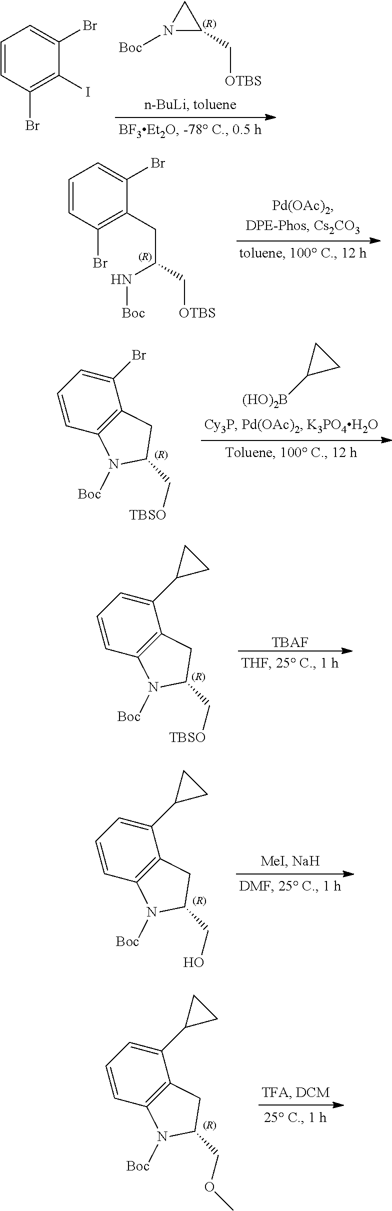

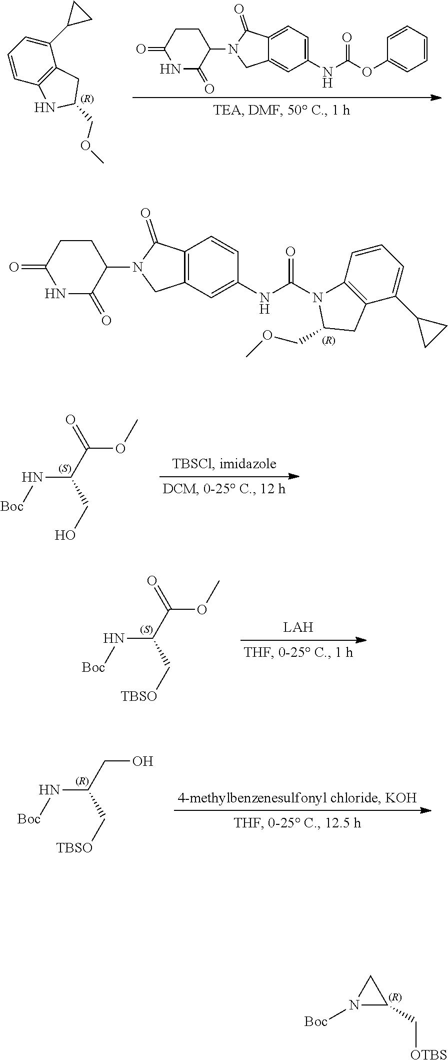

Step 1. To a solution of methyl (S)-methyl 2-((tert-butoxycarbonyl)amino)-3-hydroxypropanoate (18.0 g, 82.1 mmol, 16.7 mL, 1.00 eq) in dichloromethane (100 mL) was added imidazole (44.7 g, 656 mmol, 8.00 eq) and tert-butyl-chloro-dimethyl-silane (24.7 g, 164 mmol, 20.1 mL, 2.00 eq) at 0° C. The mixture was stirred at 25° C. for 12 h. The reaction mixture was diluted with water (300 mL) and extracted with ethyl acetate (3×300 mL). The combined organic layers were washed with brine (200 mL), dried over sodium sulfate, filtered and concentrated under reduced pressure to give a residue. The residue was purified by column chromatography (SiO2, Petroleum ether/Ethyl acetate=1/0 to 20/1) and concentrated in vacuum to give (S)-methyl 2-((tert-butoxycarbonyl)amino)-3-((tert-butyldimethylsilyl)oxy)propanoate (28.0 g, crude) as yellow oil. ¹H NMR (400 MHz, DMSO-d₆) δ=6.91 (br d, J=8.1 Hz, 1H), 4.19-4.11 (m, 1H), 3.78 (br d, J=5.5 Hz, 2H), 3.62 (s, 3H), 1.38 (s, 9H), 0.83 (s, 9H), 0.02 (s, 6H).

Step 2. To a solution of methyl (S)-methyl 2-((tert-butoxycarbonyl)amino)-3-((tert-butyldimethylsilyl)oxy) propanoate (600 mg, 1.80 mmol, 1.00 eq) in tetrahydrofuran (5.00 mL) was added lithium aluminum hydride (68.3 mg, 1.80 mmol, 1.00 eq) at 0° C. under nitrogen. The mixture was stirred at 25° C. for 1 hr. The reaction mixture was quenched by addition saturated sodium sulfate decahydrate (100 mg) and filtered to give filtrate. The filtrate was concentrated under reduced pressure to give (R)-tert-butyl (1-((tert-butyldimethylsilyl)oxy)-3-hydroxypropan-2-yl)carbamate (400 mg, crude) as colourless oil.

¹H NMR (400 MHz, DMSO-d₆) δ=6.31 (br d, J=7.8 Hz, 1H), 4.53 (t, J=5.6 Hz, 1H), 3.58-3.51 (m, 1H), 3.50-3.46 (m, 1H), 3.38-3.33 (m, 2H), 1.37 (s, 9H), 0.85 (s, 9H), 0.02 (s, 6H).

Step 3. To a solution of (R)-tert-butyl (1-((tert-butyldimethylsilyl)oxy)-3-hydroxypropan-2-yl)carbamate (1.50 g, 4.91 mmol, 1.00 eq) in tetrahydrofuran (20.0 mL) was added 4-methylbenzenesulfonyl chloride (936 mg, 4.91 mmol, 1.00 eq) at 0° C. The mixture was stirred at 0° C. for 0.5 h. Then the mixture was added potassium hydroxide (1.38 g, 24.6 mmol, 5.00 eq) at 0° C. The mixture was stirred at 25° C. for 12 h. The mixture was concentrated under reduced pressure. The residue was purified by column chromatography (SiO2, Petroleum ether/Ethyl acetate=1/0 to 30/1) and concentrated in vacuum to give (R)-tert-butyl 2-(((tert-butyldimethylsilyl)oxy) methyl)aziridine-1-carboxylate (1.00 g, 3.48 mmol, 70% yield) as brown oil. ¹H NMR (400 MHz, DMSO-d₆) δ=3.72-3.63 (m, 1H), 3.62-3.55 (m, 1H), 2.57-2.51 (m, 1H), 2.19 (d, J=6.1 Hz, 1H), 1.99 (d, J=3.8 Hz, 1H), 1.38 (s, 9H), 0.86 (s, 9H), 0.04 (d, J=2.0 Hz, 6H).

Step 4. To a solution of 1,3-dibromo-2-iodobenzene (1.00 g, 2.76 mmol, 1.00 eq) in toluene (10.0 mL) was added n-butyllithium (2.50 M, 1.33 mL, 1.20 eq) dropwise at −78° C. under nitrogen. The mixture was stirred at −78° C. for 20 min. Then to above mixture was added (R)-tert-butyl 2-(((tert-butyldimethylsilyl)oxy) methyl) aziridine-1-carboxylate (794 mg, 2.76 mmol, 1.00 eq) in toluene (0.500 mL) dropwise at −78° C. under nitrogen, then boron trifluoride diethyl etherate (784 mg, 5.53 mmol, 682 μL, 2.00 eq) was added. The mixture was stirred at −78° C. for 10 min under nitrogen. The reaction mixture was quenched by addition saturated sodium bicarbonate aqueous solution (80 mL), and extracted with ethyl acetate (3×50 mL). The combined organic layers were washed with brine (50 mL), dried over sodium sulfate, filtered and concentrated under reduced pressure to give a residue. The residue was purified by reversed phase (column: spherical C18, 20-45 um, 100 Å, SW 80, mobile phase: [water (0.1% Formic Acid)-ACN) and lyophilized to give (R)-tert-butyl (1-((tert-butyldimethylsilyl)oxy)-3-(2,6-dibromophenyl) propan-2-yl) carbamate (270 mg, 516 umol, 18% yield) as a yellow solid. ¹H NMR (400 MHz, DMSO-d₆) δ=7.60 (br d, J=8.0 Hz, 2H), 7.08-7.00 (m, 1H), 6.36 (br d, J=9.4 Hz, 1H), 3.99-3.91 (m, 1H), 3.60 (br dd, J=6.3, 9.5 Hz, 2H), 3.13-3.07 (m, 1H), 3.03-2.97 (m, 1H), 1.24 (s, 9H), 0.85 (s, 9H), 0.02 (s, 6H).

Step 5. To a mixture of (R)-tert-butyl (1-((tert-butyldimethylsilyl)oxy)-3-(2,6-dibromophenyl) propan-2-yl) carbamate (270 mg, 516 umol, 1.00 eq) in toluene (3.00 mL) was added cesium carbonate (504 mg, 1.55 mmol, 3.00 eq), [2-(2-diphenylphosphanylphenoxy)phenyl]-diphenyl-phosphane (27.7 mg, 51.6 umol, 0.100 eq) and palladium acetate (11.6 mg, 51.6 umol, 0.100 eq). The mixture was stirred at 100° C. for 12 h under nitrogen. The reaction mixture was diluted with water (30.0 mL) and extracted with ethyl acetate (3×30 mL). The combined organic layers were washed with brine (30.0 mL), dried over sodium sulfate, filtered and concentrated under reduced pressure to give a residue. The residue was purified by reversed phase (column: spherical C18, 20-45 um, 100 Å, SW 40, mobile phase: [water (0.1% Formic Acid)-ACN) and lyophilized to give (R)-tert-butyl 4-bromo-2-(((tert-butyldimethylsilyl)oxy)methyl)indoline-1-carboxylate (70.0 mg, 158 umol, 30% yield) as a brown solid. ¹H NMR (400 MHz, DMSO-d₆) δ=7.60 (br d, J=7.9 Hz, 1H), 7.13-7.02 (m, 2H), 4.49-4.37 (m, 1H), 3.80 (br d, J=5.0 Hz, 1H), 3.67 (br d, J=9.5 Hz, 1H), 3.22 (dd, J=10.3, 16.5 Hz, 1H), 2.90 (br dd, J=1.9, 16.6 Hz, 1H), 1.50 (s, 9H), 0.65 (s, 9H), −0.03 (s, 3H), −0.12 (s, 3H).

Step 6. To a solution of (R)-tert-butyl 4-bromo-2-(((tert-butyldimethylsilyl)oxy)methyl)indoline-1-carboxylate (70.0 mg, 158 umol, 1.00 eq), cyclopropylboronic acid (54.3 mg, 632 umol, 4.00 eq), tricyclohexylphosphane (8.87 mg, 31.6 umol, 10.2 uL, 0.200 eq) and potassium phosphate (100 mg, 474 umol, 3.00 eq) in toluene (3.00 mL) and water (0.300 mL) was added palladium acetate (3.55 mg, 15.8 umol, 0.100 eq). The mixture was stirred at 100° C. for 12 h under nitrogen. The reaction mixture was poured into water (40 mL) and extracted with ethyl acetate (3×20 mL). The combined organic phase was washed with brine (80 mL), dried over anhydrous sodium sulfate, filtered and concentrated in vacuum to give a residue. The residue was purified by silica gel chromatography (Petroleum ether/Ethyl acetate=1/0 to 20/1) and concentrated in vacuum to give (R)-tert-butyl 2-(((tert-butyldimethylsilyl)oxy)methyl)-4-cyclopropylindoline-1-carboxylate (60.0 mg, 148 umol, 93% yield) as colorless oil. MS (ESI) m/z. 304.1 [M+H−100]⁺

Step 7. To a solution of (R)-tert-butyl 2-(((tert-butyldimethylsilyl)oxy)methyl)-4-cyclopropylindoline-1-carboxylate (60.0 mg, 148 umol, 1.00 eq) in tetrahydrofuran (2.00 mL) was added tetrabutylammoniumfluoride (1.00 M, 178 μL, 1.20 eq), the mixture was stirred at 25° C. for 0.5 h. The reaction mixture was poured into water (60.0 mL) and extracted with ethyl acetate (3×30 mL). The combined organic phase was washed with brine (50.0 mL), dried over anhydrous sodium sulfate, filtered and concentrated in vacuum to give (R)-tert-butyl 4-cyclopropyl-2-(hydroxymethyl) indoline-1-carboxylate (60.0 mg, crude) was obtained as colorless oil. MS (ESI) m/z. 234.1 [M+H−55]⁺

Step 8. To a solution of (R)-tert-butyl 4-cyclopropyl-2-(hydroxymethyl)indoline-1-carboxylate (60.0 mg, 207 umol, 1.00 eq) in dimethyformamide (2.00 mL) was added sodium hydride (12.4 mg, 311 umol, 60% purity, 1.50 eq). The mixture was stirred at 25° C. for 10 min, then iodomethane (58.8 mg, 414 umol, 25.8 uL, 2.00 eq) was added. The mixture was stirred at 25° C. for another 50 min. The reaction mixture was poured into water (40 mL) and extracted with ethyl acetate (3×20 mL). The combined organic phase was washed with brine (80.0 mL), dried over anhydrous sodium sulfate, filtered and concentrated in vacuum to give a residue. The residue was purified by silica gel chromatography (Petroleum ether/Ethyl acetate=1/0 to 10/1) to give (R)-tert-butyl 4-cyclopropyl-2-(methoxymethyl)indoline-1-carboxylate (40.0 mg, 131 umol, 63% yield) as colorless oil. MS (ESI) m/z. 204.0 [M+H−100]⁺

Step 9. To a solution of (R)-tert-butyl 4-cyclopropyl-2-(methoxymethyl)indoline-1-carboxylate (40.0 mg, 132 umol, 1.00 eq) in dichloromethane (1.00 mL) was added trifluoroacetic acid (308 mg, 2.70 mmol, 200 μL, 20.5 eq). The mixture was stirred at 25° C. for 0.5 h. The reaction mixture was concentrated to give (R)-4-cyclopropyl-2-(methoxymethyl) indoline (30.0 mg, crude) as a yellow solid. MS (ESI) m/z. 204.0 [M+H]⁺

Step 10. To a solution of (R)-4-cyclopropyl-2-(methoxymethyl)indoline (30.0 mg, 147 umol, 1.00 eq) and triethylamine (59.7 mg, 590 umol, 82.2 uL, 4.00 eq) in dimethyformamide (2.00 mL) was added phenyl (2-(2,6-dioxopiperidin-3-yl)-1-oxoisoindolin-5-yl)carbamate (50.4 mg, 133 umol, 0.900 eq). The mixture was stirred at 50° C. for 1 h. The reaction mixture was filtered to give a filtrate. The filtrate was purified by prep-HPLC (column: Phenomenex luna C18 150*25 mm*10 um; mobile phase: [water (FA)-ACN]; B %: 39%-69%, 10 min) to give (2R)-4-cyclopropyl-N-(2-(2,6-dioxopiperidin-3-yl)-1-oxoisoindolin-5-yl)-2-(methoxymethyl)indoline-1-carboxamide (19.45 mg, 39.8 umol, 26% yield) as a white solid. ¹H NMR (400 MHz, DMSO-d₆) δ=10.97 (s, 1H), 9.16 (s, 1H), 7.81 (s, 1H), 7.65 (d, J=8.3 Hz, 1H), 7.59-7.47 (m, 2H), 7.06 (t, J=7.9 Hz, 1H), 6.49 (d, J=7.8 Hz, 1H), 5.09 (dd, J=5.0, 13.4 Hz, 1H), 5.01-4.92 (m, 1H), 4.48-4.38 (m, 1H), 4.36-4.23 (m, 1H), 3.53-3.47 (m, 2H), 3.40 (br d, J=10.0 Hz, 1H), 3.36 (s, 3H), 2.98-2.87 (m, 2H), 2.62 (br s, 1H), 2.39 (br dd, J=4.3, 13.0 Hz, 1H), 2.05-1.96 (m, 1H), 1.87-1.77 (m, 1H), 0.93 (br d, J=8.4 Hz, 2H), 0.72-0.58 (m, 2H). MS (ESI) m/z. 489.1 [M+H]⁺

### Example 102. Synthesis of Compound 331

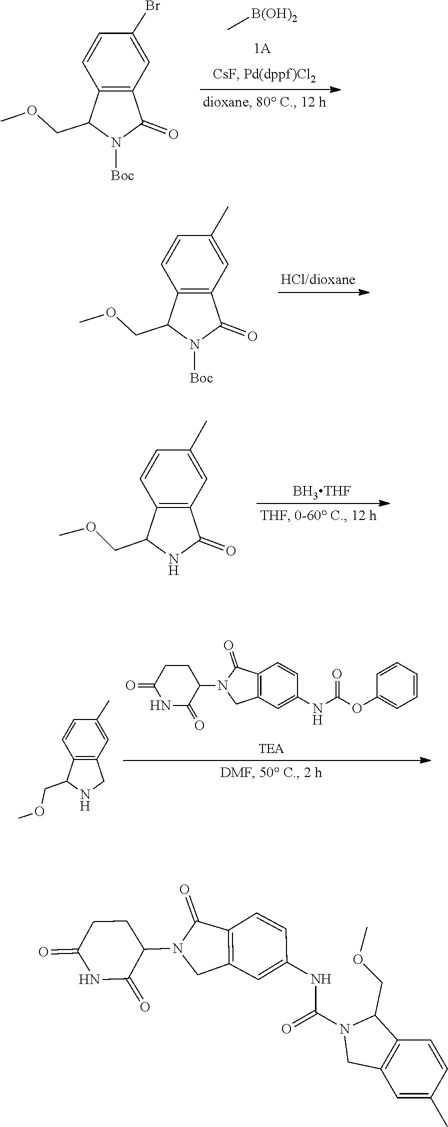

Step 1. To a solution of tert-butyl 5-bromo-1-(methoxymethyl)-3-oxo-isoindoline-2-carboxylate (1.00 g, 2.81 mmol, 1.00 eq), hydroperoxy(methyl)borane (336 mg, 5.61 mmol, 2.00 eq), cesium fluoride (1.28 g, 8.42 mmol, 310 μL, 3.00 eq) in dioxane (20.0 mL) was added (1,1-bis(diphenylphosphino)ferrocene)dichloropalladium(II) (205 mg, 281 umol, 0.100 eq) under nitrogen atmosphere. The mixture was stirred at 80° C. for 12 h under nitrogen atmosphere. The mixture was diluted with water (50 mL) and extracted with ethyl acetate (3×30 mL). The organic phase was separated, washed with brine (30 mL), dried over sodium sulfate, filtered and concentrated under reduced pressure to get a residue. The residue was purified by column chromatography (SiO₂, Petroleum ether/Ethyl acetate=30/1 to 10/1) to get tert-butyl 1-(methoxymethyl)-5-methyl-3-oxoisoindoline-2-carboxylate (650 mg, 2.23 mmol, 79% yield) as yellow oil. ¹H NMR (400 MHz, DMSO-d₆) δ=7.59-7.50 (m, 3H), 5.13 (br s, 1H), 3.93-3.89 (m, 1H), 3.87-3.81 (m, 1H), 3.14 (s, 3H), 2.40 (s, 3H), 1.52 (s, 9H). MS (ESI) m/z 192.0 [M+H−100]⁺

Step 2. To a solution of tert-butyl 1-(methoxymethyl)-5-methyl-3-oxo-isoindoline-2-carboxylate (600 mg, 2.06 mmol, 1.00 eq) in hydrochloric acid/dioxane (2.00 mL) was stirred at 25° C. for 1 h. The mixture was concentrated under reduced pressure to give 3-(methoxymethyl)-6-methylisoindolin-1-one (420 mg, crude) as pink oil. MS (ESI) m/z 192.0 [M+H]⁺

Step 3. To a solution of 3-(methoxymethyl)-6-methyl-isoindolin-1-one (400 mg, 2.09 mmol, 1.00 eq) in tetrahydrofuran (5.00 mL) was added borane-tetrahydrofuran complex (1 M, 20.9 mL, 10.0 eq) dropwise at 0° C. under nitrogen atmosphere. The mixture was stirred at 60° C. for 12 h. Methanol (30 mL) was added dropwise at 0° C. and followed by hydrochloric acid solution (6 M, 15 mL), the reaction mixture was stirred at 80° C. for 1 h. Then aqueous sodium hydroxide solution (5 M) was added to adjust the mixture to pH=7. The mixture was extracted with ethyl acetate (3×30 mL). The combined organic layers were washed with brine (30 mL), dried over anhydrous sodium sulfate, filtered and concentrated under reduced pressure to give a residue. The residue was purified by reversed-phase HPLC (column: spherical C18, 20-45 um, 100 Å, SW 120, mobile phase: [water (0.1% Formic Acid)-acetonitrile]) and lyophilized to get 1-(methoxymethyl)-5-methyl-isoindoline (130 mg, crude) as pink oil. MS (ESI) m/z 178.0 [M+H]⁺

Step 4. To a solution of 1-(methoxymethyl)-5-methyl-isoindoline (50.0 mg, 282 umol, 1.00 eq) and phenyl (2-(2,6-dioxopiperidin-3-yl)-1-oxoisoindolin-5-yl)carbamate (85.6 mg, 226 umol, 0.800 eq) in N,N-dimethyl formamide (1.00 mL) was added triethylamine (85.6 mg, 846 umol, 118 μL, 3.00 eq). The mixture was stirred at 50° C. for 2 h. The mixture was adjusted to pH=5-6 with formic acid and filtered. The filtrate was purified by reversed phase HPLC (column: spherical C 18, 20-45 um, 100 Å, SW 40, mobile phase: [water (0.1% Formic Acid)-acetonitrile) and lyophilized to afford N-(2-(2,6-dioxopiperidin-3-yl)-1-oxoisoindolin-5-yl)-1-(methoxymethyl)-5-methylisoindoline-2-carboxamide (50.3 mg, 108 umol, 38% yield, 99% purity) as a white solid. ¹H NMR (400 MHz, DMSO-d₆) δ=10.96 (s, 1H), 8.86 (br s, 1H), 7.86 (s, 1H), 7.63-7.54 (m, 2H), 7.27 (d, J=7.6 Hz, 1H), 7.16-7.09 (m, 2H), 5.28 (br s, 1H), 5.07 (dd, J=5.2, 13.6 Hz, 1H), 4.86-4.76 (m, 1H), 4.73-4.63 (m, 1H), 4.44-4.36 (m, 1H), 4.32-4.22 (m, 1H), 3.75-3.65 (m, 2H), 3.25 (s, 3H), 2.95-2.85 (m, 1H), 2.63-2.59 (m, 1H), 2.37 (br dd, J=4.4, 13.2 Hz, 1H), 2.32 (s, 3H), 2.04-1.94 (m, 1H). MS (ESI) m/z 463.2 [M+H]⁺

### Example 103. Synthesis of Compound 324

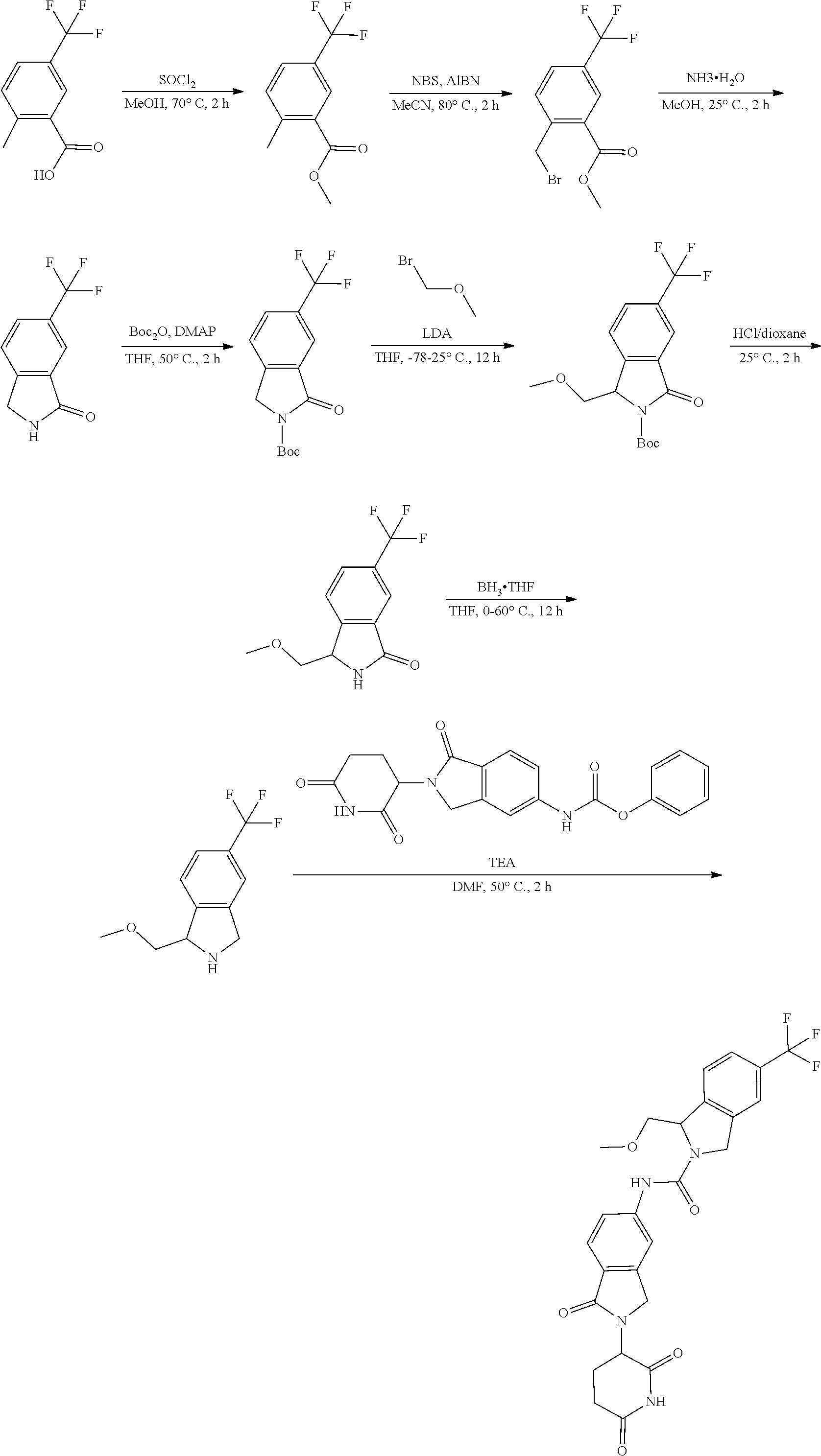

Step 1. To a solution of 2-methyl-5-(trifluoromethyl)benzoic acid (6.30 g, 30.8 mmol, 1.00 eq) in methanol (60.0 mL) was added thionyl chloride (11.0 g, 92.6 mmol, 6.72 mL, 3.00 eq). The mixture was stirred at 70° C. for 2 h. The mixture was diluted with water (50 mL) and extracted with ethyl acetate (3×30 mL). The reaction mixture was filtered and concentrated under reduced pressure to give a residue. The residue was purified by column chromatography (SiO₂, Petroleum ether/Ethyl acetate=10/1 to 0/1) to get methyl 2-methyl-5-(trifluoromethyl)benzoate (6.60 g, 30.2 mmol, 98% yield) as yellow oil. ¹H NMR (400 MHz, DMSO-d₆) δ=8.05 (s, 1H), 7.83-7.79 (m, 1H), 7.56 (d, J=8.0 Hz, 1H), 3.85 (s, 3H), 2.57 (s, 3H).

Step 2. To a solution of methyl 2-methyl-5-(trifluoromethyl)benzoate (6.30 g, 28.9 mmol, 1.00 eq) in acetonitrile (20.0 mL) was added 1-bromopyrrolidine-2,5-dione (5.65 g, 31.7 mmol, 1.10 eq) and 2-(2-cyanopropan-2-yldiazenyl)-2-methylpropanenitrile (474 mg, 2.89 mmol, 0.100 eq) under nitrogen atmosphere. The mixture was stirred at 80° C. for 2 h. The mixture was concentrated under reduced pressure. The residue was purified by column chromatography (SiO₂, Petroleum ether/Ethyl acetate=1/0) to get methyl 2-(bromomethyl)-5-(trifluoromethyl)benzoate (4.00 g, 13.5 mmol, 47% yield) as light yellow oil. ¹H NMR (400 MHz, DMSO-d₆) δ=8.25 (s, 1H), 7.76 (d, J=8.0 Hz, 1H), 7.63 (d, J=8.0 Hz, 1H), 4.98 (s, 2H), 3.99 (s, 3H).

Step 3. To a solution of methyl 2-(bromomethyl)-5-(trifluoromethyl)benzoate (4.00 g, 13.5 mmol, 1.00 eq) in methanol (40.0 mL) was added ammonium hydroxide (13.9 g, 108 mmol, 15.4 mL, 37% purity, 8.00 eq). The mixture was stirred at 25° C. for 2 h. The mixture was concentrated under reduced pressure. The residue was purified by column chromatography (SiO₂, Petroleum ether/Ethyl acetate=2/1) to get 6-(trifluoromethyl)isoindolin-1-one (2.40 g, 11.9 mmol, 89% yield) as a white solid. ¹H NMR (400 MHz, CDCl₃) δ=8.85 (br s, 1H), 7.96 (d, J=8.0 Hz, 1H), 7.93 (s, 1H), 7.83 (d, J=8.0 Hz, 1H), 4.49 (s, 2H). MS (ESI) m/z 202.3 [M+H]⁺

Step 4. To a solution of 6-(trifluoromethyl)isoindolin-1-one (2.40 g, 11.9 mmol, 1.00 eq), di-tert-butyl dicarbonate (5.21 g, 23.8 mmol, 5.48 mL, 2.00 eq) in tetrahydrofuran (30.0 mL) was added 4-dimethylaminopyridin (728 mg, 5.97 mmol, 0.500 eq). The mixture was stirred at 50° C. for 2 h. The reaction mixture was diluted with water (30 mL) and extracted with ethyl acetate (2×30 mL). The organic phase was separated, washed with brine (10 mL), dried over anhydrous sodium sulfate, filtered and concentrated under reduced pressure to get tert-butyl 1-oxo-6-(trifluoromethyl)isoindoline-2-carboxylate (4.30 g, crude) as a yellow solid. ¹H NMR (400 MHz, DMSO-d₆) δ=8.08 (br d, J=8.0 Hz, 1H), 8.03 (s, 1H), 7.90 (d, J=8.0 Hz, 1H), 4.89 (s, 2H), 1.53 (s, 9H). MS (ESI) m/z 246.1 [M+H−56]⁺

Step 5. To a solution of tert-butyl 1-oxo-6-(trifluoromethyl)isoindoline-2-carboxylate (1.50 g, 4.98 mmol, 1.00 eq) in tetrahydrofuran (15.0 mL) was added lithium diisopropylamide (2 M, 3.73 mL, 1.50 eq) dropwise at −78° C. under nitrogen atmosphere. Then the reaction mixture was stirred at −78° C. for 1 h. Then the reaction mixture was added bromo(methoxy)methane (1.24 g, 9.96 mmol, 813 μL, 2.00 eq) dropwise at −78° C. under nitrogen atmosphere. Then the reaction mixture was stirred at 25° C. for 11 h under nitrogen atmosphere. The reaction mixture was quenched by addition saturated ammonium chloride solution (30 mL) at 0° C., and extracted with ethyl acetate (3×50 mL). The combined organic layers were washed with brine (30 mL), dried over anhydrous sodium sulfate, filtered and concentrated under reduced pressure to give a residue. The residue was purified by reversed-phase HPLC (column: spherical C18, 20-45 um, 100 Å, SW 120, mobile phase: [water (0.1% formic acid)-acetonitrile]) and lyophilized to get tert-butyl 1-(methoxymethyl)-3-oxo-5-(trifluoromethyl)isoindoline-2-carboxylate (500 mg, 1.45 mmol, 29% yield) as yellow oil. ¹H NMR (400 MHz, CDCl₃) δ=8.16 (s, 1H), 7.88 (d, J=8.0 Hz, 1H), 7.74 (d, J=8.0 Hz, 1H), 5.16 (dd, J=3.2, 6.4 Hz, 1H), 4.09 (dd, J=3.2, 9.2 Hz, 1H), 3.62 (dd, J=6.8, 9.2 Hz, 1H), 3.35 (s, 3H), 1.62 (s, 9H). MS (ESI) m/z 246.2 [M+H−100]⁺

Step 6. To a solution of tert-butyl 1-(methoxymethyl)-3-oxo-5-(trifluoromethyl)isoindoline-2-carboxylate (450 mg, 1.30 mmol, 1.00 eq) in hydrochloric acid/dioxane (3.00 mL) was stirred at 25° C. for 0.5 h. The mixture was concentrated in vacuum to get 3-(methoxymethyl)-6-(trifluoromethyl)isoindolin-1-one (450 mg, crude) as pink oil. MS (ESI) m/z 246.2 [M+H]⁺

Step 7. To a solution of 3-(methoxymethyl)-6-(trifluoromethyl)isoindolin-1-one (450 mg, 1.84 mmol, 1.00 eq) in tetrahydrofuran (5.00 mL) was added borane-tetrahydrofuran complex (1 M, 27.5 mL, 15.0 eq) dropwise at 0° C. under nitrogen atmosphere. The mixture was stirred at 60° C. for 12 h. Methanol (30 mL) was added dropwise at 0° C. and followed by hydrochloric acid solution (6 M, 15 mL), the reaction mixture was stirred at 80° C. for 1 h. Then aqueous sodium hydroxide solution (5 M) was added to adjust the mixture to pH=7. The mixture was extracted with ethyl acetate (3×30 mL). The combined organic layers were washed with brine (30 mL), dried over anhydrous sodium sulfate, filtered and concentrated under reduced pressure to give a residue. The residue was purified by reversed-phase HPLC (column: spherical C18, 20-45 um, 100 Å, SW 80, mobile phase: [water (0.1% formic acid)-acetonitrile]) and concentrated to get 1-(methoxymethyl)-5-(trifluoromethyl)isoindoline (110 mg, 476 mol, 63% yield) as yellow oil. MS (ESI) m/z 232.2 [M+H]⁺

Step 8. To a solution of 1-(methoxymethyl)-5-(trifluoromethyl)isoindoline (50.0 mg, 216 mol, 1.00 eq), phenyl (2-(2,6-dioxopiperidin-3-yl)-1-oxoisoindolin-5-yl)carbamate (82.0 mg, 216 mol, 1.00 eq) in N,N-dimethyl formamide (3.00 mL) was added triethylamine (65.6 mg, 648 mol, 90.3 μL, 3.00 eq). The mixture was stirred at 50° C. for 2 h. The mixture was adjusted to pH=5-6 with formic acid and filtered. The filtrate was purified by reversed phase HPLC (column: spherical C 18, 20-45 um, 100 Å, SW 40, mobile phase: [water (0.1% formic acid)-acetonitrile) and lyophilized to afford N-(2-(2,6-dioxopiperidin-3-yl)-1-oxoisoindolin-5-yl)-1-(methoxymethyl)-5-(trifluoromethyl)isoindoline-2-carboxamide (44.6 mg, 84.6 mol, 39% yield, 98% purity) as a white solid. ¹H NMR (400 MHz, DMSO-d₆) δ=10.97 (s, 1H), 8.93 (s, 1H), 7.87 (s, 1H), 7.75 (s, 1H), 7.72-7.67 (m, 1H), 7.66-7.57 (m, 3H), 5.44 (br s, 1H), 5.08 (dd, J=5.0, 13.4 Hz, 1H), 5.00-4.89 (m, 1H), 4.85-4.76 (m, 1H), 4.45-4.37 (m, 1H), 4.32-4.24 (m, 1H), 3.78 (br s, 2H), 3.25 (s, 3H), 2.97-2.84 (m, 1H), 2.63-2.56 (m, 1H), 2.38 (br dd, J=4.0, 12.8 Hz, 1H), 2.04-1.94 (m, 1H). MS (ESI) m/z 517.2 [M+H]⁺

### Example 104. Synthesis of Compound 346

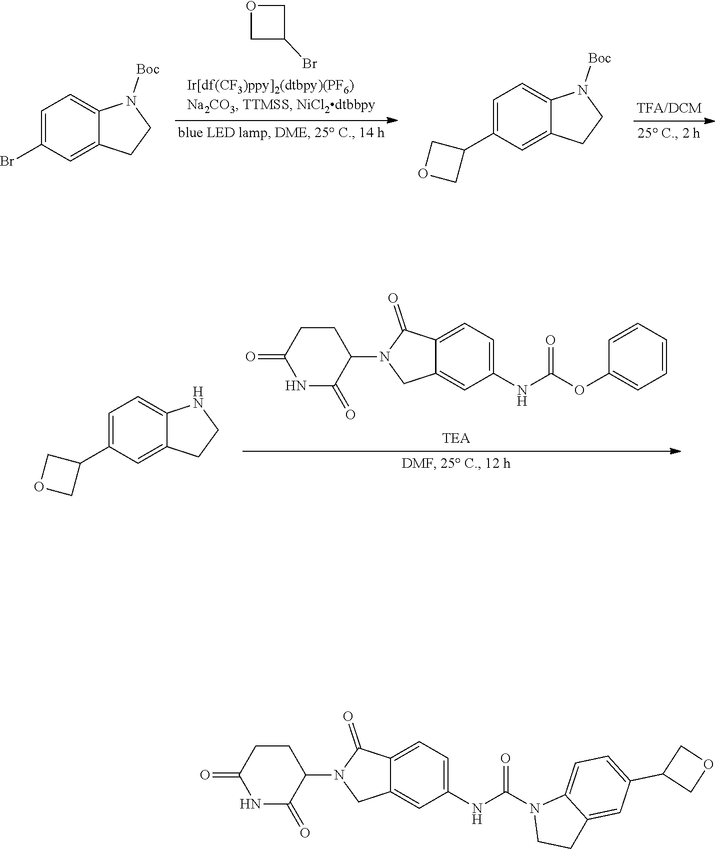 [SMILES: CC(C)(C)OC(=O)N1CCc2cc(Br)ccc21 ; BrC1COC1 ; CC(C)(C)OC(=O)N1CCc2cc(C3COC3)ccc21 ; c1cc2c(cc1C1COC1)CCN2 ; O=C1CCC(N2Cc3cc(NC(=O)Oc4ccccc4)ccc3C2=O)C(=O)N1 ; O=C1CCC(N2Cc3cc(NC(=O)N4CCc5cc(C6COC6)ccc54)ccc3C2=O)C(=O)N1]

Step 1. To an 40 mL vial equipped with a stir bar was added tert-butyl 5-bromoindoline-1-carboxylate (3.00 g, 10.1 mmol, 1.00 eq), 3-bromooxetane (1.79 g, 13.1 mmol, 1.30 eq), Ir[dF(CF₃)ppy]₂(dtbpy)(PF₆) (113 mg, 101 umol, 0.0100 eq), (4,4′-bis(1,1-dimethylethyl)-2,2′-bipyridine)nickel(II) dichloride (60.1 mg, 151 umol, 0.0150 eq), tris-(trimethylsilyl)silane (2.50 g, 10.1 mmol, 3.10 mL, 1.00 eq), sodium carbonate (2.13 g, 20.1 mmol, 2.00 eq) in methoxymethane (10.0 mL). The vial was sealed and placed under nitrogen was added. The reaction was stirred and irradiated with a 10 W blue LED lamp (3 cm away), with cooling water to keep the reaction temperature at 25° C. for 14 h. The mixture was diluted with water (100 mL) and extracted with ethyl acetate (3×100 mL). The organic layers were collected and was washed with brine (100.0 mL), dried over anhydrous sodium sulfate and evaporated to dryness. The crude product was purified by column chromatography on silica gel eluted with petroleum ether/ethyl acetate=1/0 to 10/1 to afford tert-butyl 5-(oxetan-3-yl)indoline-1-carboxylate (1.00 g, 3.63 mmol, 36% yield) as yellow oil. ¹H NMR (400 MHz, DMSO-d₆) δ=7.80-7.46 (m, 1H), 7.26 (s, 1H), 7.14 (br d, J=8.4 Hz, 1H), 4.90 (dd, J=5.8, 8.2 Hz, 2H), 4.56 (dd, J=5.8, 6.8 Hz, 2H), 4.17 (t, J=7.2 Hz, 1H), 3.90 (t, J=8.6 Hz, 2H), 3.06 (br t, J=8.6 Hz, 2H), 1.50 (s, 9H).

Step 2. To a solution of trifluoroacetic acid (1.54 g, 13.5 mmol, 1.00 mL, 7.44 eq) in dichloromethane (5.00 mL) was added tert-butyl 5-(oxetan-3-yl)indoline-1-carboxylate (0.500 g, 1.82 mmol, 1.00 eq). The mixture was stirred at 25° C. for 2 h. The mixture was concentrated under reduced pressure. The residue was added toluene (15 mL) and concentrated under reduced pressure. The residue was added toluene (10 mL) and concentrated under reduced pressure to afford 5-(oxetan-3-yl)indoline (800 mg, crude) as brown oil. ¹H NMR (400 MHz, DMSO-d₆) δ=7.35 (s, 1H), 7.24 (br d, J=7.6 Hz, 1H), 7.19-7.14 (m, 1H), 4.94 (br dd, J=6.0, 8.3 Hz, 2H), 4.59 (br t, J=6.3 Hz, 2H), 4.32-4.23 (m, 1H), 3.73-3.68 (m, 2H), 3.24-3.16 (m, 2H). MS (ESI) m/z 176.1 [M+H]⁺

Step 3. To a solution of phenyl (2-(2,6-dioxopiperidin-3-yl)-1-oxoisoindolin-5-yl)carbamate (520 mg, 1.37 mmol, 0.300 eq), 5-(oxetan-3-yl)indoline (0.800 g, 4.57 mmol, 1.00 eq) in dimethyformamide (2.00 mL) was added triethylamine (1.85 g, 18.3 mmol, 2.54 mL, 4.00 eq). The mixture was stirred at 25° C. for 12 h. The mixture was filtered to give filtrate. The filtrate was purified by reversed-phase HPLC (column: spherical C18, 20-45 um, 100 Å, SW 40, mobile phase: [water (0.1% Formic Acid)-ACN]) and further purified by prep-HPLC (column: Phenomenex luna C18 150*25 mm*10 um; mobile phase: [water (FA)-ACN]; B %: 17%-47%, 10 min) to afford N-(2-(2,6-dioxopiperidin-3-yl)-1-oxoisoindolin-5-yl)-5-(oxetan-3-yl)indoline-1-carboxamide (22.5 mg, 47.5 umol, 1% yield, 97% purity) as a white solid. ¹H NMR (400 MHz, DMSO-d₆) δ=10.97 (s, 1H), 8.83 (s, 1H), 7.92 (s, 1H), 7.85 (d, J=8.3 Hz, 1H), 7.71-7.59 (m, 2H), 7.29 (s, 1H), 7.15 (d, J=8.4 Hz, 1H), 5.09 (dd, J=5.0, 13.2 Hz, 1H), 4.91 (dd, J=5.8, 8.2 Hz, 2H), 4.59 (t, J=6.3 Hz, 2H), 4.46-4.40 (m, 1H), 4.32-4.26 (m, 1H), 4.23-4.12 (m, 3H), 3.20 (br t, J=8.6 Hz, 2H), 2.98-2.87 (m, 1H), 2.60 (br dd, J=2.2, 15.2 Hz, 1H), 2.39 (dq, J=4.4, 13.2 Hz, 1H), 2.05-1.96 (m, 1H). MS (ESI) m/z 460.9 [M+H]⁺

### Example 105. Synthesis of Compound 348

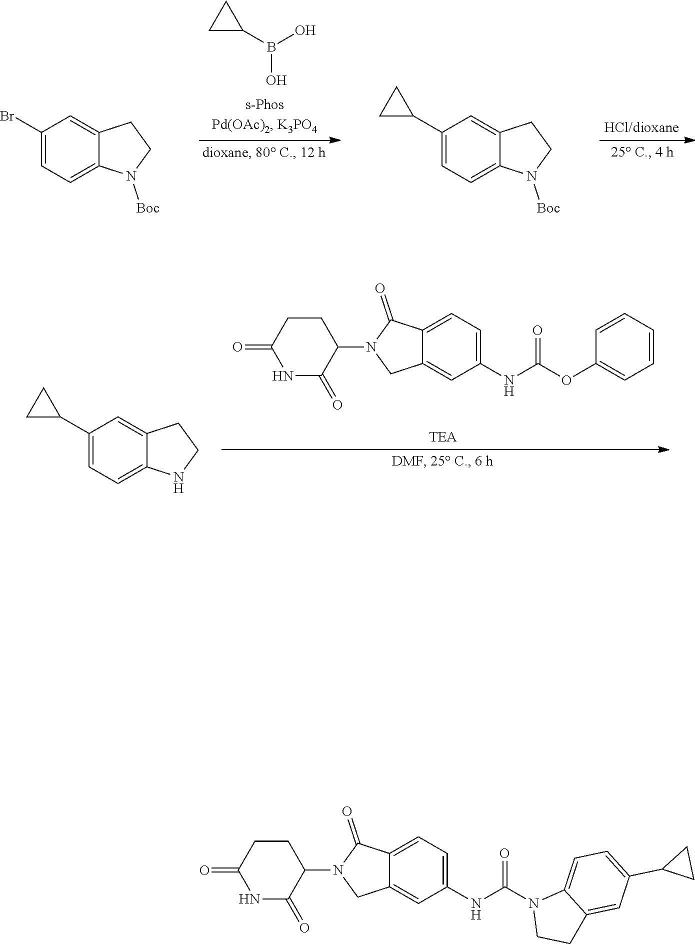 [SMILES: OB(O)C1CC1 ; CC(C)(C)OC(=O)N1CCc2cc(Br)ccc21 ; CC(C)(C)OC(=O)N1CCc2cc(C3CC3)ccc21 ; c1cc2c(cc1C1CC1)CCN2 ; O=C1CCC(N2Cc3cc(NC(=O)Oc4ccccc4)ccc3C2=O)C(=O)N1 ; O=C1CCC(N2Cc3cc(NC(=O)N4CCc5cc(C6CC6)ccc54)ccc3C2=O)C(=O)N1]

Step 1. To a solution of tert-butyl 5-bromoindoline-1-carboxylate (1.00 g, 3.35 mmol, 1.00 eq), cyclopropylboronic acid (432 mg, 5.03 mmol, 1.50 eq), potassium phosphate (2.14 g, 10.0 mmol, 3.00 eq) in 1,4-dioxane (10.0 mL) was added dicyclohexyl(2′,6′-dimethoxy-[1,1′-biphenyl]-2-yl)phosphine (275 mg, 670 umol, 0.200 eq) and palladium(II) acetate (150 mg, 670 umol, 0.200 eq). The mixture was stirred at 80° C. for 12 h under nitrogen. The mixture was filtered to give a filter liquor, then it was concentrated under reduced pressure to give a residue. The residue was purified by reversed-phase HPLC (column: spherical C18, 20-45 um, 100 Å, SW 80, mobile phase: [water-acetonitrile) to give tert-butyl 5-cyclopropylindoline-1-carboxylate (860 mg, 3.32 mmol, 98% yield) as a white solid. ¹H NMR (400 MHz, DMSO-d₆) δ=7.68-7.42 (m, 1H), 6.95-6.77 (m, 2H), 3.92-3.79 (m, 2H), 2.99 (br t, J=8.6 Hz, 2H), 1.88-1.79 (m, 1H), 1.52-1.46 (m, 9H), 0.90-0.83 (m, 2H), 0.61-0.54 (m, 2H). MS (ESI) m/z 204.0 [M−56+1]⁺

Step 2. A solution of tert-butyl 5-cyclopropylindoline-1-carboxylate (500 mg, 1.93 mmol, 1.00 eq) in 1,4-dioxane (5.00 mL) was added dioxane/hydrochloride (10.0 mL) stirred at 25° C. for 4 h. The reaction mixture was concentrated under reduced pressure to give 5-cyclopropylindoline (300 mg, 1.88 mmol, 97% yield) as a white solid.

Step 3. To a solution of 5-cyclopropylindoline (150 mg, 942 umol, 1.00 eq), triethylamine (285 mg, 2.83 mmol, 393 μL, 3.00 eq) in dimethyl formamide (5.00 mL) was added phenyl (2-(2,6-dioxopiperidin-3-yl)-1-oxoisoindolin-5-yl)carbamate (357 mg, 942 umol, 1.00 eq). The mixture was stirred at 25° C. for 6 h. The reaction mixture was concentrated under reduced pressure to give a residue which was purified by reversed-phase HPLC (column: spherical C18, 20-45 um, 100 Å, SW 80, mobile phase: [water (0.1% Formic Acid)-acetonitrile) to give 5-cyclopropyl-N-(2-(2,6-dioxopiperidin-3-yl)-1-oxoisoindolin-5-yl)indoline-1-carboxamide (105 mg, 237 umol, 25% yield) as an off-white solid. ¹H NMR (400 MHz, DMSO-d₆) δ=10.97 (s, 1H), 8.78 (s, 1H), 7.91 (s, 1H), 7.74 (d, J=8.3 Hz, 1H), 7.64 (s, 2H), 6.91 (s, 1H), 6.87 (br d, J=8.4 Hz, 1H), 5.09 (dd, J=5.1, 13.3 Hz, 1H), 4.49-4.36 (m, 1H), 4.34-4.24 (m, 1H), 4.14 (t, J=8.6 Hz, 2H), 3.13 (br t, J=8.4 Hz, 2H), 2.98-2.85 (m, 1H), 2.68-2.56 (m, 1H), 2.38 (dq, J=4.6, 13.4 Hz, 1H), 2.04-1.95 (m, 1H), 1.91-1.81 (m, 1H), 0.92-0.84 (m, 2H), 0.64-0.55 (m, 2H). MS (ESI) m/z 444.9 [M+H]⁺

### Example 106. Synthesis of Compound 345

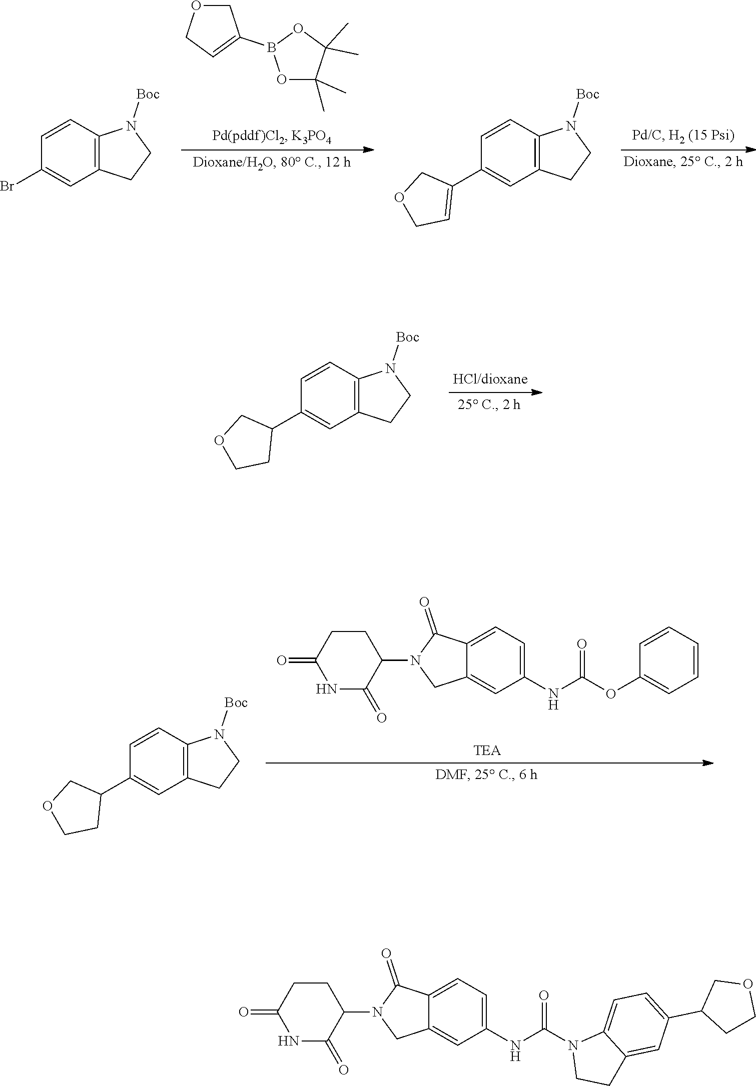

Step 1. To a solution of tert-butyl 5-bromoindoline-1-carboxylate (912 mg, 3.06 mmol, 1.20 eq) in dioxane (5.00 mL) and water (0.500 mL) was added 2-(2,5-dihydrofuran-3-yl)-4,4,5,5-tetramethyl-1,3,2-dioxaborolane (500 mg, 2.55 mmol, 1.00 eq), potassium phosphate (1.62 g, 7.65 mmol, 3.00 eq) and [1,1′-bis (diphenylphosphino)ferrocene]dichloropalladium(II) (186 mg, 255 umol, 0.10 eq) under nitrogen. The mixture was stirred at 80° C. for 12 h. The reaction mixture was diluted with water (30 mL) and extracted with ethyl acetate (2×30 mL). The organic phase was separated, washed with brine (10 mL), dried over sodium sulfate, filtered and concentrated under reduced pressure to give a residue. The residue was purified by column chromatography (SiO2, Petroleum ether/Ethyl acetate=8/1 to 3/1) to give tert-butyl 5-(2,5-dihydrofuran-3-yl)indoline-1-carboxylate (400 mg, 1.39 mmol, 54% yield) as a yellow solid.

¹H NMR (400 MHz, DMSO-d₆) δ=7.77-7.53 (m, 1H), 7.29 (s, 1H), 7.17 (br d, J=8.2 Hz, 1H), 6.33 (s, 1H), 4.84 (br d, J=3.8 Hz, 2H), 4.70 (br d, J=3.8 Hz, 2H), 3.90 (br t, J=8.6 Hz, 2H), 3.05 (br t, J=8.6 Hz, 2H), 1.50 (s, 9H).

Step 2. To a solution of tert-butyl 5-(2,5-dihydrofuran-3-yl)indoline-1-carboxylate (400 mg, 1.39 mmol, 1.00 eq) in methanol (10.0 mL) was added palladium on carbon (50 mg, 10% purity) under hydrogen (15 Psi). The mixture was stirred at 25° C. for 2 h. The mixture was filtered to give a filter liquor, then it was concentrated under reduced pressure to give tert-butyl 5-(tetrahydrofuran-3-yl)indoline-1-carboxylate (250 mg, crude) as a yellow solid. MS (ESI) m/z 234.2 [M−56+H]⁺

Step 3. A solution of tert-butyl 5-(tetrahydrofuran-3-yl)indoline-1-carboxylate (250 mg, 863 umol, 1.00 eq) in hydrochloric acid/dioxane (5.00 mL) was stirred at 25° C. for 2 h. The reaction mixture was concentrated under reduced pressure to give 5-(tetrahydrofuran-3-yl)indoline (178 mg, crude) as a yellow solid. ¹H NMR (400 MHz, DMSO-d₆) δ=11.60 (br dd, J=10.6, 14.2 Hz, 1H), 7.42-7.30 (m, 2H), 7.30-7.25 (m, 1H), 4.01 (t, J=7.8 Hz, 1H), 3.96 (br s, 1H), 3.79 (q, J=7.6 Hz, 1H), 3.68 (br t, J=7.8 Hz, 2H), 3.55 (br d, J=7.6 Hz, 1H), 3.46-3.39 (m, 1H), 3.16 (br t, J=7.8 Hz, 2H), 2.31 (dtd, J=4.6, 7.7, 12.3 Hz, 1H), 1.90 (qd, J=7.9, 12.2 Hz, 1H).

Step 4. To a solution of phenyl (2-(2,6-dioxopiperidin-3-yl)-1-oxoisoindolin-5-yl)carbamate (360 mg, 951 umol, 1.50 eq) in dimethyl formamide (1.00 mL) was added 5-(tetrahydrofuran-3-yl)indoline5-(tetrahydrofuran-3-yl)indoline (120 mg, 634 umol, 1.00 eq), triethylamine (192 mg, 1.90 mmol, 264 μL, 3.00 eq). The mixture was stirred at 25° C. for 12 h. The reaction mixture was diluted with water (10 mL). The solution was filtered to give a filter cake. The filter cake was purified by reversed-phase HPLC (column: spherical C18, 20-45 μm, 100 Å, SW 120, mobile phase: [water (0.1% Formic Acid)-ACN]) and lyophilized to give N-(2-(2,6-dioxopiperidin-3-yl)-1-oxoisoindolin-5-yl)-5-(tetrahydrofuran-3-yl)indoline-1-carboxamide (210 mg, 443 umol, 69% yield) as a white solid. ¹H NMR (400 MHz, DMSO-d₆) δ=10.98 (s, 1H), 8.82 (s, 1H), 7.92 (s, 1H), 7.79 (d, J=8.2 Hz, 1H), 7.64 (s, 2H), 7.13 (s, 1H), 7.04 (br d, J=8.2 Hz, 1H), 5.09 (dd, J=5.2, 13.2 Hz, 1H), 4.48-4.38 (m, 1H), 4.33-4.25 (m, 1H), 4.16 (t, J=8.6 Hz, 2H), 4.00 (t, J=7.8 Hz, 1H), 3.93 (dt, J=4.4, 8.4 Hz, 1H), 3.78 (q, J=8.0 Hz, 1H), 3.49 (t, J=8.0 Hz, 1H), 3.32-3.28 (m, 1H), 3.16 (br t, J=8.4 Hz, 2H), 2.97-2.85 (m, 1H), 2.60 (br dd, J=2.0, 15.2 Hz, 1H), 2.44-2.32 (m, 1H), 2.27 (tdd, J=3.8, 7.8, 11.8 Hz, 1H), 2.05-1.96 (m, 1H), 1.88 (qd, J=8.2, 12.2 Hz, 1H). MS (ESI) m/z 475.1 [M+H]⁺

### Example 107. Synthesis of Compound 349

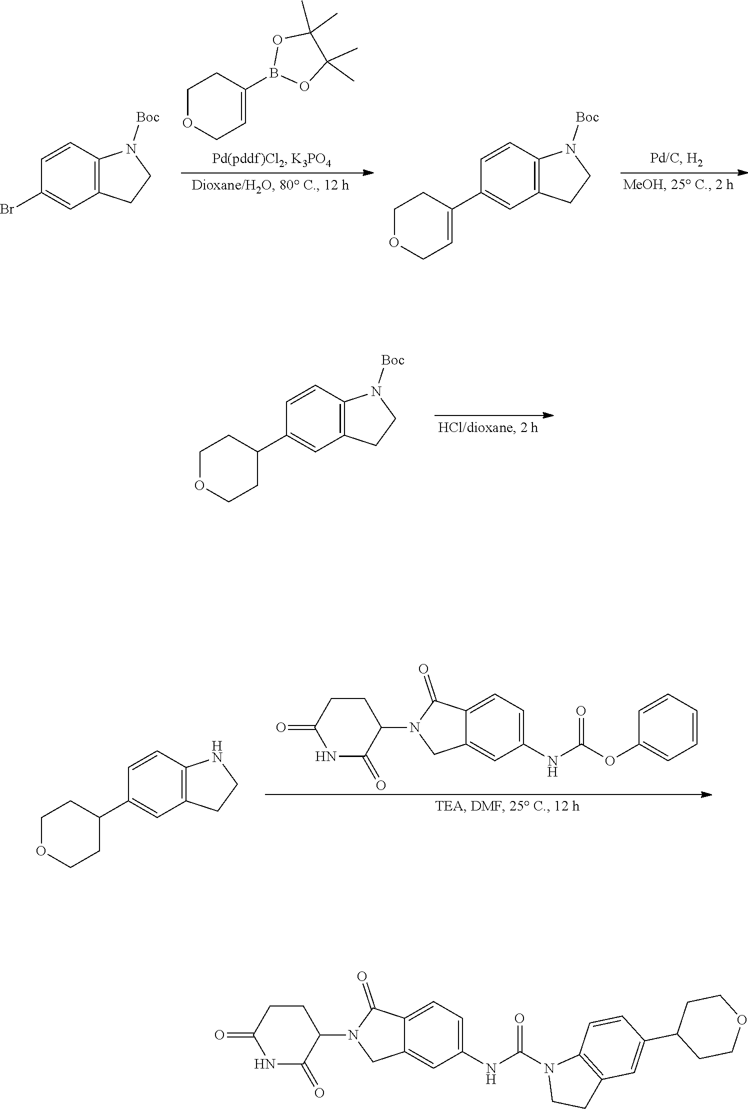

Step 1. To a mixture of tert-butyl 5-bromoindoline-1-carboxylate (500 mg, 1.68 mmol, 1.00 eq) and 2-(3,6-dihydro-2H-pyran-4-yl)-4,4,5,5-tetramethyl-1,3,2-dioxaborolane (422 mg, 2.01 mmol, 1.20 eq) in dioxane (5.00 mL) and water (1.00 mL) was added potassium phosphate (1.07 g, 5.03 mmol, 3.00 eq) and [1,1-bis(diphenylphosphino)ferrocene]dichloropalladium(II) (122.70 mg, 167.69 umol, 0.100 eq). The mixture was stirred at 80° C. for 12 h under nitrogen. The reaction mixture was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by reversed-phase HPLC (column: spherical C18, 20-45 um, 100 Å, SW 120, mobile phase: [water (0.1% Formic Acid)-ACN) and lyophilized to give tert-butyl 5-(3,6-dihydro-2H-pyran-4-yl)indoline-1-carboxylate (350 mg, 1.16 mmol, 69% yield) as a brown solid. ¹H NMR (400 MHz, DMSO-d₆) δ=7.72-7.48 (m, 1H), 7.29 (s, 1H), 7.22 (br d, J=8.3 Hz, 1H), 6.14 (br s, 1H), 4.19 (br d, J=2.5 Hz, 2H), 3.90 (br t, J=8.7 Hz, 2H), 3.80 (t, J=5.4 Hz, 2H), 3.05 (br t, J=8.6 Hz, 2H), 2.40 (br d, J=1.4 Hz, 2H), 1.50 (s, 9H).

Step 2. To a solution of tert-butyl 5-(3,6-dihydro-2H-pyran-4-yl)indoline-1-carboxylate (350 mg, 1.16 mmol, 1.00 eq) in methanol (20.0 mL) was added palladium on carbon (400 mg, 10% purity) under nitrogen atmosphere. The suspension was degassed and purged with hydrogen for 3 times. The mixture was stirred under hydrogen (15 Psi) at 25° C. for 2 h. The mixture was filtered. The filtrate was concentrated in vacuum to give tert-butyl 5-(tetrahydro-2H-pyran-4-yl)indoline-1-carboxylate (370 mg, crude) as a white solid. ¹H NMR (400 MHz, DMSO-d₆) δ=7.66-7.48 (m, 1H), 7.08 (s, 1H), 7.00 (br d, J=8.4 Hz, 1H), 3.96-3.81 (m, 4H), 3.40 (dt, J=3.5, 11.1 Hz, 2H), 3.02 (br t, J=8.6 Hz, 2H), 2.73-2.64 (m, 1H), 1.68-1.56 (m, 4H), 1.49 (s, 9H).

Step 3. A mixture of tert-butyl 5-(tetrahydro-2H-pyran-4-yl)indoline-1-carboxylate (370 mg, 1.22 mmol, 1.00 eq) in hydrochloric acid (4 M in dioxane, 5.00 mL) was stirred at 25° C. for 2 h. The mixture was concentrated in vacuum to give 5-(tetrahydro-2H-pyran-4-yl)indoline (285 mg, crude) as a white solid. MS (ESI) m/z. 204.1 [M+H]⁺

Step 4. To a mixture of 5-(tetrahydro-2H-pyran-4-yl)indoline (100 mg, 492 umol, 1.20 eq) and phenyl (2-(2,6-dioxopiperidin-3-yl)-1-oxoisoindolin-5-yl)carbamate (155 mg, 409 umol, 1.00 eq) in dimethyformamide (2.00 mL) was added triethylamine (124 mg, 1.23 mmol, 171 μL, 3.00 eq). The mixture was stirred at 25° C. for 12 h. The mixture was added water (20 mL) and filtered. The filter cake was added water (10 mL) and lyophilized. The compound was purified by reversed-phase HPLC (column: spherical C18, 20-45 um, 100 Å, SW 80, mobile phase: [water (0.1% Formic Acid)-ACN) and lyophilized) to give N-(2-(2,6-dioxopiperidin-3-yl)-1-oxoisoindolin-5-yl)-5-(tetrahydro-2H-pyran-4-yl)indoline-1-carboxamide (56.81 mg, 115 umol, 28% yield, 99% purity) as an off-white solid. ¹H NMR (400 MHz, DMSO-d₆) δ=10.97 (s, 1H), 8.81 (s, 1H), 7.92 (s, 1H), 7.79 (d, J=8.3 Hz, 1H), 7.69-7.58 (m, 2H), 7.11 (s, 1H), 7.02 (dd, J=1.2, 8.2 Hz, 1H), 5.09 (dd, J=5.1, 13.3 Hz, 1H), 4.47-4.38 (m, 1H), 4.33-4.26 (m, 1H), 4.15 (t, J=8.6 Hz, 2H), 3.93 (br dd, J=2.6, 10.2 Hz, 2H), 3.46-3.39 (m, 2H), 3.16 (br t, J=8.5 Hz, 2H), 2.97-2.87 (m, 1H), 2.69 (td, J=5.1, 10.3 Hz, 1H), 2.64-2.57 (m, 1H), 2.44-2.33 (m, 1H), 2.03-1.94 (m, 1H), 1.69-1.58 (m, 4H). MS (ESI) m/z. 489.4 [M+H]⁺

### Example 108. Synthesis of Compound 344

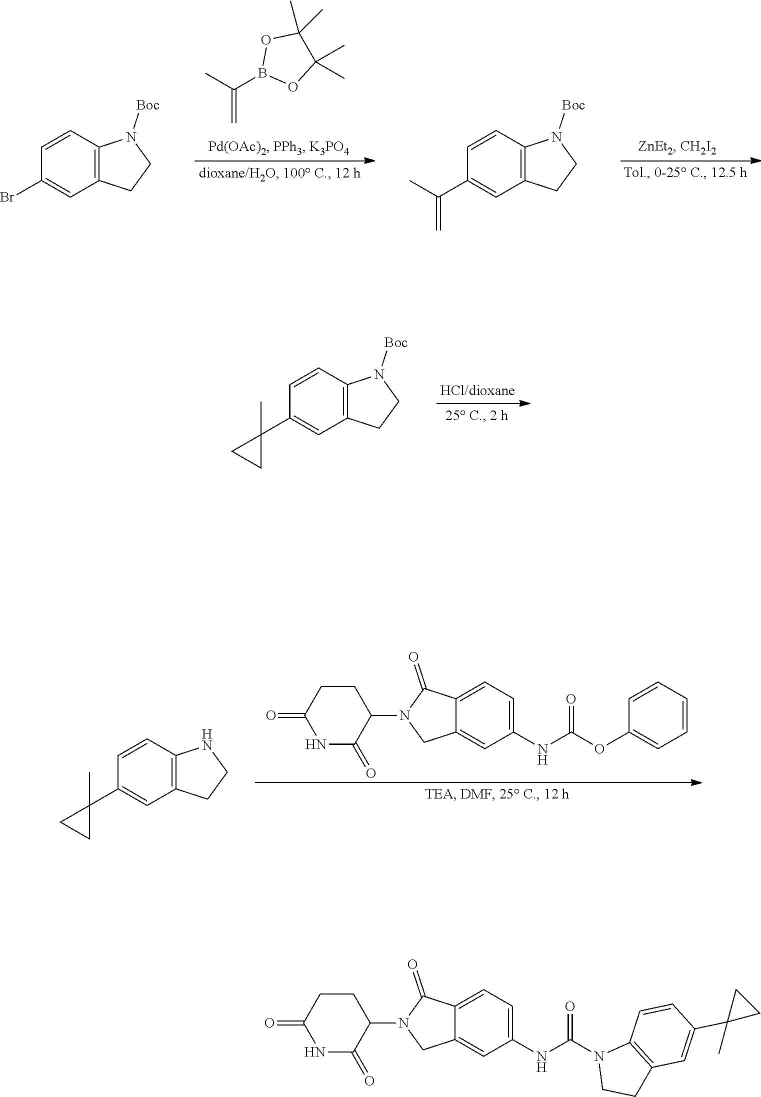

Step 1. To a solution of tert-butyl 5-bromoindoline-1-carboxylate (2.00 g, 6.71 mmol, 1.00 eq), potassium phosphate (4.27 g, 20.1 mmol, 3.00 eq) in dioxane (100 mL) and water (10.0 mL) was added triphenylphosphine (352 mg, 1.34 mmol, 0.200 eq), 4,4,5,5-tetramethyl-2-(prop-1-en-2-yl)-1,3,2-dioxaborolane (3.38 g, 20.1 mmol, 3.00 eq) and palladium diacetate (301 mg, 1.34 mmol, 0.200 eq) under nitrogen. The mixture was stirred at 100° C. for 12 h under nitrogen. The mixture was diluted with water (100 mL) and extracted with ethyl acetate (3×50 mL). The organic layers were collected and was washed with brine (100 mL), dried over anhydrous sodium sulfate and evaporated to dryness. The crude product was purified by column chromatography on silica gel eluted with petroleum ether/ethyl acetate=1/0 to 25/1 to afford tert-butyl 5-(prop-1-en-2-yl)indoline-1-carboxylate (1.50 g, 5.78 mmol, 86% yield) as an off-white solid. ¹H NMR (400 MHz, DMSO-d₆) δ=7.69-7.51 (m, 1H), 7.34 (s, 1H), 7.28 (br d, J=8.3 Hz, 1H), 5.33 (s, 1H), 4.98 (s, 1H), 3.92-3.86 (m, 2H), 3.05 (br t, J=8.6 Hz, 2H), 2.07 (s, 3H), 1.50 (s, 9H).

Step 2. To a solution of diethylzine (2.00 M, 7.71 mL, 4.00 eq) in toluene (40.0 mL) was added a solution of diiodomethane (2.07 g, 7.71 mmol, 622 μL, 2.00 eq) in toluene (20.0 mL) dropwise at 0° C. under nitrogen. The mixture was stirred at 0° C. for 0.5 h. Then to above mixture was added tert-butyl 5-(prop-1-en-2-yl)indoline-1-carboxylate (1.00 g, 3.86 mmol, 1.00 eq) in toluene (10.0 mL) at 0° C. under nitrogen. The mixture was stirred at 25° C. for 12 h. The mixture was diluted with water (150 mL) and extracted with ethyl acetate (3×100 mL). The organic layers were collected and was washed with brine (100 mL), dried over anhydrous sodium sulfate and evaporated to dryness. The crude product was purified by column chromatography on silica gel eluted with petroleum ether/ethyl acetate=1/0 to 200/1 to afford tert-butyl 5-(1-methylcyclopropyl)indoline-1-carboxylate (800 mg, crude) as brown oil. ¹H NMR (400 MHz, DMSO-d₆) δ=7.62-7.49 (m, 1H), 7.05 (br s, 1H), 6.99 (br d, J=8.4 Hz, 1H), 3.88-3.83 (m, 2H), 3.08-3.03 (m, 2H), 1.49 (br s, 9H), 1.32 (d, J=1.4 Hz, 3H), 0.76-0.73 (m, 2H), 0.67 (br d, J=1.8 Hz, 2H).

Step 3. The tert-butyl 5-(1-methylcyclopropyl)indoline-1-carboxylate (100 mg, 366 umol, 1.00 eq) was added in hydrochloric acid (4.00 M in dioxane) (2.00 mL). The mixture was stirred at 25° C. for 2 h. The mixture was concentrated under reduced pressure to afford 5-(1-methylcyclopropyl)indoline (100 mg, crude) as brown oil. MS (ESI) m/z 174.1 [M+H]⁺

Step 4. To a solution of phenyl (2-(2,6-dioxopiperidin-3-yl)-1-oxoisoindolin-5-yl)carbamate (65.7 mg, 173 umol, 1.00 eq), 5-(1-methylcyclopropyl)indoline (30.0 mg, 4.57 mmol, 1.00 eq) in dimethyformamide (2.00 mL) was added triethylamine (70.1 mg, 693 umol, 96.4 uL, 4.00 eq). The mixture was stirred at 25° C. for 12 h. The mixture was added formic acid (0.5 mL) to adjusted pH=3-4. The mixture was purified by reversed-phase HPLC (column: spherical C18, 20-45 um, 100 Å, SW 40, mobile phase: [water (0.1% formic acid)-acetonitrile]) and lyophilized to give N-(2-(2,6-dioxopiperidin-3-yl)-1-oxoisoindolin-5-yl)-5-(1-methylcyclopropyl)indoline-1-carboxamide (22.3 mg, 48.1 umol, 27% yield, 99% purity) as a white solid. ¹H NMR (400 MHz, DMSO-d₆) δ=10.97 (br s, 1H), 8.79 (s, 1H), 7.91 (s, 1H), 7.76 (d, J=8.4 Hz, 1H), 7.64 (s, 2H), 7.08 (s, 1H), 7.01 (d, J=8.3 Hz, 1H), 5.09 (dd, J=5.1, 13.3 Hz, 1H), 4.48-4.38 (m, 1H), 4.34-4.25 (m, 1H), 4.15 (t, J=8.6 Hz, 2H), 3.15 (br t, J=8.4 Hz, 2H), 2.98-2.85 (m, 1H), 2.69-2.59 (m, 1H), 2.41-2.28 (m, 1H), 2.00 (br dd, J=4.8, 10.4 Hz, 1H), 1.35 (s, 3H), 0.81-0.74 (m, 2H), 0.72-0.66 (m, 2H). MS (ESI) m/z 459.3 [M+H]⁺

### Example 109. Synthesis of Compound 330

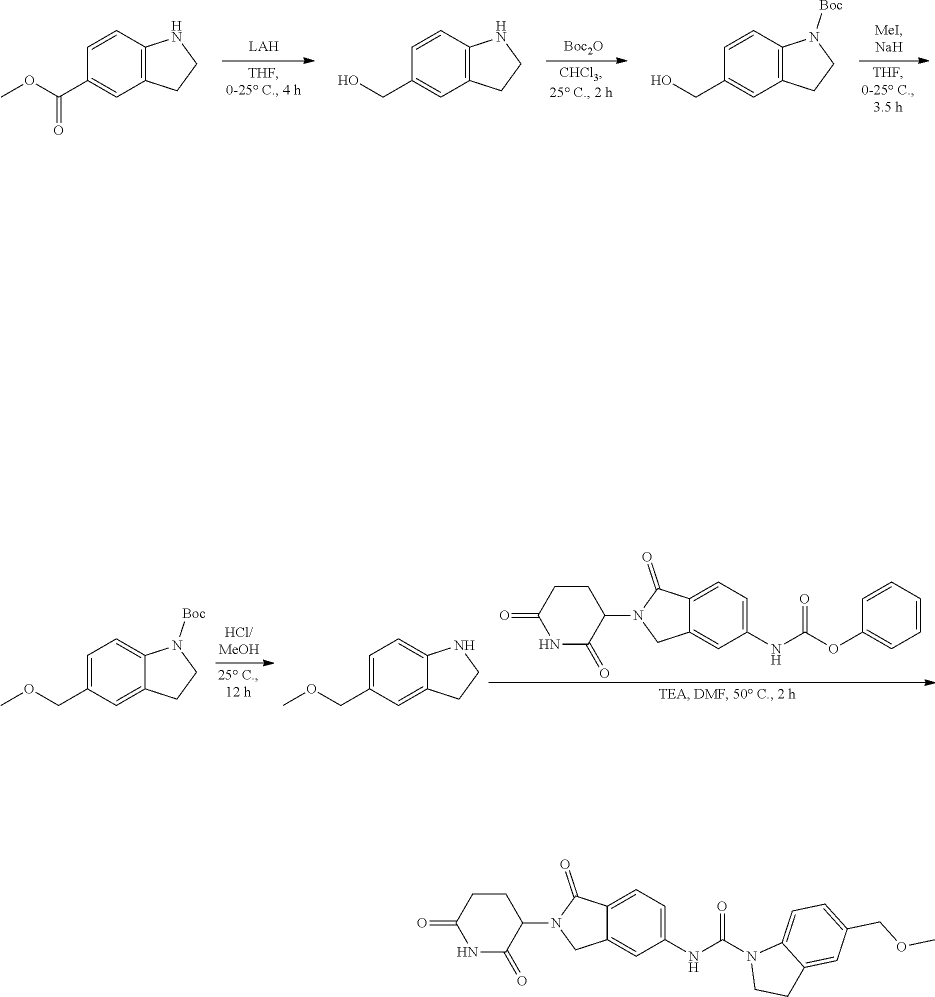

Step 1. To a solution of methyl indoline-5-carboxylate (4.00 g, 22.5 mmol, 1.00 eq) in tetrahydrofuran (100 mL) was added lithium aluminum hydride (1.71 g, 45.2 mmol, 2.00 eq) under nitrogen at 0° C. The mixture was stirred at 25° C. for 4 h. Water (1 ml) was added to quench the reaction at 0° C., followed by 15 wt % sodium hydroxide (1 ml) and another portion of water (3 mL). The reaction mixture was diluted with water (30 mL) and exacted with ethyl acetate (3×90.0 mL). The organic phase was separated, washed with brine (10 mL), dried over sodium sulfate, filtered and concentrated under reduced pressure to give a residue. The residue was purified by column chromatography (SiO₂, Petroleum ether/Ethyl acetate=8/1 to 0/1). The crude product was purified by reversed-phase HPLC (column: spherical C18, 20-45 um, 100 Å, SW 330, mobile phase: [water-ACN]) and lyophilized to afford indolin-5-ylmethanol (310 mg, 2.08 mmol, 9.2% yield) as a white solid.

¹H NMR (400 MHz, DMSO-d₆) δ=6.97 (s, 1H), 6.83 (d, J=7.9 Hz, 1H), 6.42 (d, J=7.8 Hz, 1H), 5.35 (br s, 1H), 4.79 (t, J=5.6 Hz, 1H), 4.30 (d, J=5.5 Hz, 2H), 3.38 (t, J=8.4 Hz, 2H), 2.86 (t, J=8.4 Hz, 2H).

Step 2. To a solution of indolin-5-ylmethanol (310 mg, 2.08 mmol, 1.00 eq) in trichloromethane (10.0 mL) was added di-tert-butyldicarbonate (680 mg, 3.12 mmol, 716 μL, 1.50 eq) at 25° C. for 2 h. The reaction mixture was diluted with water (30 mL) and exacted with ethyl acetate (3×90.0 mL). The organic phase was separated, washed with brine (10 mL), dried over sodium sulfate, filtered and concentrated under reduced pressure to give a residue. The residue was purified by column chromatography (SiO2, Petroleum ether/Ethyl acetate=8/1 to 0/1) to give tert-butyl 5-(hydroxymethyl)indoline-1-carboxylate (915 mg, crude) as yellow oil. ¹H NMR (400 MHz, DMSO-d₆) δ=7.60 (br s, 1H), 7.13 (s, 1H), 7.06 (d, J=8.1 Hz, 1H), 5.04 (t, J=5.7 Hz, 1H), 4.41 (d, J=5.6 Hz, 2H), 3.89 (t, J=8.7 Hz, 2H), 3.03 (br t, J=8.6 Hz, 2H), 1.50 (s, 9H).

Step 3. To a solution of tert-butyl 5-(hydroxymethyl)indoline-1-carboxylate (815 mg, 3.27 mmol, 1.00 eq) in tetrahydrofuran (10.0 mL) was added sodium hydride (261 mg, 6.54 mmol, 60% purity, 2.00 eq) at 0° C. under nitrogen. The mixture was stirred at 0° C. for 0.5 h. Then iodomethane (928 mg, 6.54 mmol, 407 μL, 2.00 eq) was added at 0° C. under nitrogen. The mixture was stirred at 25° C. for 3 h. The reaction mixture was quenched with formic acid (0.5 ml) to give a solution. The residue was purified by column chromatography (SiO2, Petroleum ether/Ethyl acetate=8/1 to 0/1) to afford tert-butyl 5-(methoxymethyl)indoline-1-carboxylate (790 mg, 3.00 mmol, 91% yield) as yellow solid. ¹H NMR (400 MHz, DMSO-d₆) δ=7.78-7.38 (m, 1H), 7.13 (s, 1H), 7.07 (br d, J=8.1 Hz, 1H), 4.31 (s, 2H), 3.90 (t, J=8.7 Hz, 2H), 3.23 (s, 3H), 3.04 (br t, J=8.6 Hz, 2H), 1.50 (s, 9H).

Step 4. To a solution of tert-butyl 5-(methoxymethyl)indoline-1-carboxylate (50.0 mg, 189 umol, 1.00 eq) in methanol (5.00 mL) was added concentrated hydrochloric acid (100 uL), the mixture was stirred at 25° C. for 12 h. The reaction mixture was concentrated to give 5-(methoxymethyl)indoline (30.0 mg, crude) as a yellow solid.

Step 5. A mixture of 5-(methoxymethyl)indoline (20.0 mg, 122 umol, 1.00 eq), phenyl (2-(2,6-dioxopiperidin-3-yl)-1-oxoisoindolin-5-yl)carbamate (46.5 mg, 122 umol, 1.00 eq) and triethylamine (37.2 mg, 367 umol, 51.2 uL, 3.00 eq) in dimethylformamide (1.00 mL) was stirred at 50° C. for 2 h. The reaction mixture was filtered to give a filtrate. The filtrate was purified by Prep-HPLC (column: Waters xbridge 150*25 mm 10 um; mobile phase: [water (NH4HCO3)-ACN]; B %: 16%-46%, 8 min) to afford N-(2-(2,6-dioxopiperidin-3-yl)-1-oxoisoindolin-5-yl)-5-(methoxymethyl)indoline-1-carboxamide (12.39 mg, 27.6 umol, 22% yield) as a white solid. ¹H NMR (400 MHz, DMSO-d₆) δ=10.98 (br s, 1H), 8.92-8.80 (m, 1H), 7.93 (s, 1H), 7.87-7.81 (m, 1H), 7.68-7.62 (m, 2H), 7.17 (s, 1H), 7.09 (d, J=8.4 Hz, 1H), 5.10 (dd, J=5.1, 13.1 Hz, 1H), 4.49-4.41 (m, 1H), 4.37-4.27 (m, 3H), 4.19 (t, J=8.7 Hz, 2H), 3.26 (s, 3H), 3.19 (br t, J=8.5 Hz, 2H), 2.98-2.88 (m, 1H), 2.61-2.59 (m, 1H), 2.45-2.33 (m, 1H), 2.07-1.96 (m, 1H). MS (ESI) m/z 447.0 [M−H]⁺

### Example 110. Synthesis of Compound 341

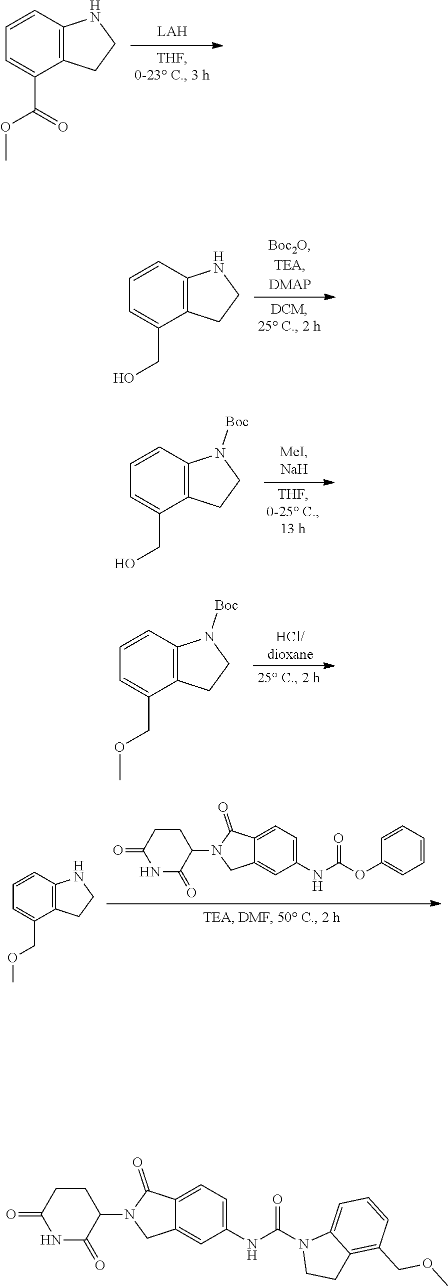

Step 1. To a solution of methyl indoline-4-carboxylate (2.00 g, 11.3 mmol, 1.00 eq) in tetrahydrofuran (40.0 mL) was added lithium aluminum hydride (428 mg, 11.3 mmol, 1.00 eq) in one portion at 0° C. The mixture was stirred at 23° C. for 3 h under nitrogen atmosphere. The mixture was quenched with sodium sulfate decahydrate, then diluted with water (30.0 mL) and extracted with ethyl acetate (2×30.0 mL). The organic phase was separated, washed with brine (10.0 mL), dried over sodium sulfate, filtered and concentrated under reduced pressure to give indolin-4-ylmethanol (1.70 g, crude) as yellow oil. ¹H NMR (400 MHz, DMSO-d₆) δ=6.86 (t, J=7.6 Hz, 1H), 6.54 (d, J=7.6 Hz, 1H), 6.38 (d, J=7.6 Hz, 1H), 5.38 (br s, 1H), 4.36 (d, J=5.6 Hz, 2H), 3.39-3.36 (m, 2H), 2.86 (t, J=8.4 Hz, 2H)

Step 2. To a solution of indolin-4-ylmethanol (1.70 g, 11.4 mmol, 1.00 eq) in dichloromethane (20.0 mL) was added di-tert-butyl dicarbonate (2.49 g, 11.4 mmol, 2.62 mL, 1.00 eq), 4-dimethylaminopyridin (139 mg, 1.14 mmol, 0.100 eq), triethylamine (3.46 g, 34.2 mmol, 4.76 mL, 3.00 eq). The mixture was stirred at 25° C. for 2 h. The mixture was filtered to give a filtrate. The residue was purified by column chromatography (SiO₂, Petroleum ether/Ethyl acetate=1/0 to 5/1) and further purified by column chromatography (SiO₂, Petroleum ether/Ethyl acetate=1/0 to 10/1) to get tert-butyl 4-(hydroxymethyl)indoline-1-carboxylate (1.00 g, 4.01 mmol, 35% yield) as colorless oil. ¹H NMR (400 MHz, DMSO-d₆) δ=7.76-7.56 (m, 1H), 7.20-7.12 (m, 1H), 6.91 (d, J=7.6 Hz, 1H), 5.01 (s, 2H), 3.93-3.90 (m, 2H), 3.05 (br t, J=8.8 Hz, 2H), 1.42 (s, 9H).

Step 3. To a solution of tert-butyl 4-(hydroxymethyl)indoline-1-carboxylate (450 mg, 1.81 mmol, 1.00 eq) in tetrahydrofuran (10.0 mL) was added sodium hydride (72.2 mg, 1.81 mmol, 60% purity, 1.00 eq) at 0° C. under nitrogen atmosphere. The mixture was stirred at 0° C. for 1 h. Then was added methyl iodide (512 mg, 3.61 mmol, 225 μL, 2.00 eq) at 0° C. under nitrogen atmosphere. The mixture was stirred at 25° C. for 12 h under nitrogen atmosphere. The reaction mixture was adjust pH to 5-6 with formic acid to give a solution and concentrated under reduced pressure to give a residue. The residue was purified by column chromatography (SiO₂, Petroleum ether/Ethyl acetate=1/0 to 20/1) to get tert-butyl 4-(methoxymethyl) indoline-1-carboxylate (200 mg, 760 umol, 21% yield) as a white solid.

¹H NMR (400 MHz, DMSO-d₆) δ=7.75-7.32 (m, 1H), 7.13 (t, J=7.6 Hz, 1H), 6.89 (d, J=7.6 Hz, 1H), 4.35 (s, 2H), 3.91 (t, J=8.8 Hz, 2H), 3.27 (s, 3H), 3.02 (t, J=8.8 Hz, 2H), 1.50 (s, 9H).

Step 4. To a solution of tert-butyl 4-(methoxymethyl)indoline-1-carboxylate (150 mg, 570 umol, 1.00 eq) in hydrochloric acid/dioxane (2.00 mL). The mixture was stirred at 25° C. for 2 h. The reaction mixture was concentrated under reduced pressure to give 4-(methoxymethyl)indoline (100 mg, 501 umol, 88% yield, hydrochloric acid) as brown oil. MS (ESI) m/z 164.2 [M+H]⁺

Step 5. To a solution of 4-(methoxymethyl)indoline (100 mg, 501 umol, 1.00 eq, hydrochloric acid) in dimethyl formamide (2.00 mL) was added phenyl (2-(2,6-dioxopiperidin-3-yl)-1-oxoisoindolin-5-yl)carbamate (190 mg, 501 umol, 1.00 eq) and triethylamine (152 mg, 1.50 mmol, 209 μL, 3.00 eq). The mixture was stirred at 50° C. for 2 h. The mixture was filtered to give a filtrate, then was concentrated under reduced pressure and purified by reversed-phase HPLC (column: spherical C18, 20-45 um, 100 Å, SW 80, mobile phase: [water (0.1% Formic Acid)-acetonitrile]) and lyophilized to give N-(2-(2,6-dioxopiperidin-3-yl)-1-oxoisoindolin-5-yl)-4-(methoxymethyl)indoline-1-carboxamide (71.42 mg, 159 umol, 32% yield, 99% purity) as a white solid. ¹H NMR (400 MHz, DMSO-d₆) δ=10.97 (br s, 1H), 8.84 (s, 1H), 7.92 (s, 1H), 7.84 (d, J=8.0 Hz, 1H), 7.70-7.60 (m, 2H), 7.14 (t, J=8.0 Hz, 1H), 6.91 (d, J=7.6 Hz, 1H), 5.09 (dd, J=5.2, 13.2 Hz, 1H), 4.47-4.40 (m, 1H), 4.38 (s, 2H), 4.33-4.26 (m, 1H), 4.19 (t, J=8.8 Hz, 2H), 3.30 (s, 3H), 3.17 (t, J=8.8 Hz, 2H), 2.98-2.84 (m, 1H), 2.62-2.57 (m, 1H), 2.45-2.32 (m, 1H), 2.05-1.95 (m, 1H). MS (ESI) m/z 449.2 [M+H]⁺

### Example 111. Synthesis of Compound 343

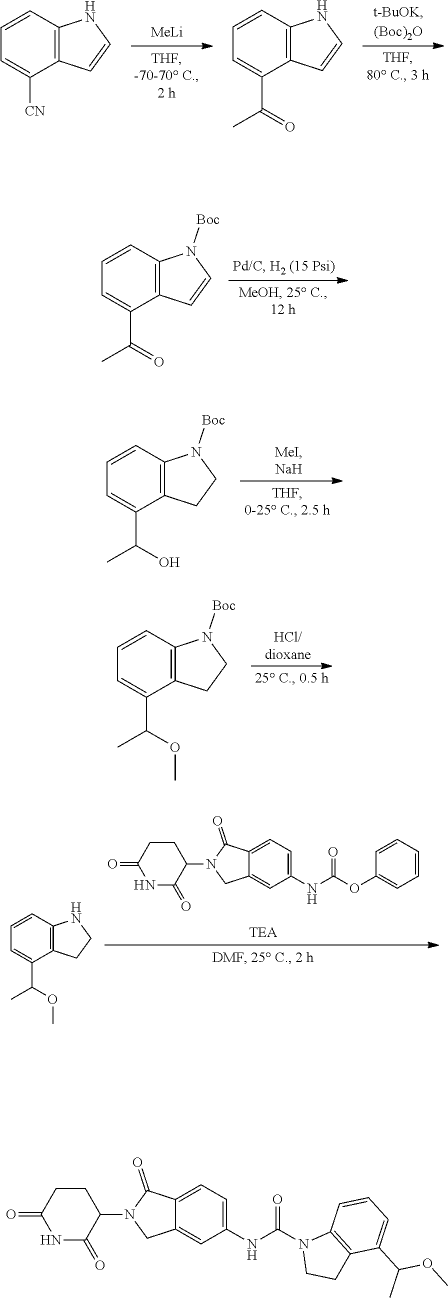

Step 1. To a mixture of methyllithium (1.6 M, 43.9 mL, 2.00 eq) in tetrahydrofuran (30.0 mL) was added a solution of 1H-indole-4-carbonitrile (5.00 g, 35.1 mmol, 1.00 eq) in tetrahydrofuran (10.0 mL) dropwise at −70° C. under nitrogen. The mixture was stirred at 70° C. for 2 h. The mixture was quenched with 1 M hydrochloric acid (100 mL) and extracted with ethyl acetate (3×80 mL). The combined organic layer was washed with brine (40 mL), dried over sodium sulfate, filtered and concentrated in vacuum to afford 1-(1H-indol-4-yl)ethanone (4.00 g, 25.1 mmol, 71% yield) as a brown solid. ¹H NMR (400 MHz, DMSO-d₆) δ=11.43 (br s, 1H), 7.76 (d, J=7.6 Hz, 1H), 7.69 (d, J=8.0 Hz, 1H), 7.53 (t, J=2.8 Hz, 1H), 7.21 (t, J=7.6 Hz, 1H), 7.08 (t, J=2.0 Hz, 1H), 2.64 (s, 3H).

Step 2. To a solution of 1-(1H-indol-4-yl)ethanone (2.00 g, 12.5 mmol, 1.00 eq) in tetrahydrofuran (20.0 mL) was added di-tert-butyldicarbonate (3.02 g, 13.8 mmol, 3.18 mL, 1.10 eq) and potassium tert-butoxide (140 mg, 1.26 mmol, 0.100 eq). Then the reaction mixture was stirred at 80° C. for 3 h. The reaction mixture was diluted with water (50 mL) and extracted with ethyl acetate (3×50 mL). The combined organic layers were washed with brine (3×20 mL), dried over sodium sulfate, filtered and concentrated under reduced pressure to give a residue. The residue was purified by column chromatography (SiO2, Petroleum ether/Ethyl acetate=20/1 to 2/1) and concentrated in vacuum to afford tert-butyl 4-acetyl-1H-indole-1-carboxylate (1.10 g, 4.03 mmol, 32% yield, 95% purity) was obtained as yellow oil. ¹H NMR (400 MHz, CDCl₃) δ=8.44 (br d, J=8.4 Hz, 1H), 7.82 (d, J=7.6 Hz, 1H), 7.73 (d, J=3.6 Hz, 1H), 7.44 (d, J=3.6 Hz, 1H), 7.38 (t, J=8.0 Hz, 1H), 2.71 (s, 3H), 1.69 (s, 9H).

Step 3. To a solution of tert-butyl 4-acetyl-1H-indole-1-carboxylate (1.00 g, 3.86 mmol, 1.00 eq) in methanol (50.0 mL) was added Palladium on activated carbon (500 mg, 10% purity). Then the reaction mixture was stirred at 25° C. for 12 h under hydrogen atmosphere (15 Psi). The reaction mixture was filtered. The filtrate was concentrated in vacuum to afford tert-butyl 4-(1-hydroxyethyl)indoline-1-carboxylate (1.00 g, 3.46 mmol, 89% yield, 91% purity) as yellow oil. ¹H NMR (400 MHz, DMSO-d₆) δ=7.67-7.37 (m, 1H), 7.12 (t, J=8.0 Hz, 1H), 7.00 (d, J=7.6 Hz, 1H), 5.10 (br s, 1H), 4.72 (q, J=6.4 Hz, 1H), 3.90 (td, J=6.9, 10.0 Hz, 2H), 3.08-2.96 (m, 2H), 1.50 (s, 9H), 1.27 (d, J=6.4 Hz, 3H).

Step 4. To a solution of tert-butyl 4-(1-hydroxyethyl)indoline-1-carboxylate (600 mg, 2.28 mmol, 1.00 eq) in tetrahydrofuran (10.0 mL) was added sodium hydride (136 mg, 3.42 mmol, 60% purity, 1.50 eq) at 0° C. Then the reaction mixture was stirred at 25° C. for 0.5 h under nitrogen atmosphere. Then the reaction mixture was added iodomethane (970 mg, 6.84 mmol, 425 μL, 3.00 eq) at 0° C. Then the reaction mixture was stirred at 25° C. for 2 h under nitrogen atmosphere. The reaction mixture was quenched by addition saturated ammonium chloride (100 mL) and extracted with ethyl acetate (3×50 mL). The combined organic layers were washed with brine (3×20 mL), dried over sodium sulfate, filtered and concentrated under reduced pressure to give a residue. The residue was purified by column chromatography (SiO2, Petroleum ether/Ethyl acetate=20/1 to 5/1) and concentrated in vacuum to afford tert-butyl 4-(1-methoxyethyl)indoline-1-carboxylate (600 mg, 1.95 mmol, 85% yield, 90% purity) as yellow oil. ¹H NMR (400 MHz, DMSO-d₆) δ=7.75-7.37 (m, 1H), 7.16 (t, J=7.6 Hz, 1H), 6.87 (d, J=7.6 Hz, 1H), 4.34 (q, J=6.4 Hz, 1H), 3.96-3.89 (m, 2H), 3.11 (s, 3H), 3.08-2.98 (m, 2H), 1.50 (s, 9H), 1.29 (d, J=6.4 Hz, 3H).

Step 5. To a solution of tert-butyl 4-(1-methoxyethyl)indoline-1-carboxylate (300 mg, 1.08 mmol, 1.00 eq) in hydrochloric acid (4 M in dioxane) (2.00 mL) was stirred at 25° C. for 0.5 h. The reaction mixture was concentrated in vacuum to afford 4-(1-methoxyethyl)indoline (250 mg, crude) as a yellow solid.

Step 6. To a solution of 4-(1-methoxyethyl)indoline (90.0 mg, 507 umol, 1.00 eq) and phenyl (2-(2,6-dioxopiperidin-3-yl)-1-oxoisoindolin-5-yl)carbamate (192 mg, 507 umol, 1.00 eq) in N,N-dimethylformamide (3.00 mL) was added triethylamine (154 mg, 1.52 mmol, 212 μL, 3.00 eq). Then the reaction mixture was stirred at 25° C. for 2 h. The reaction mixture was adjust pH to 5-6 by formic acid (0.2 mL) and filtered. The filtrate was purified by Prep-HPLC (column: Phenomenex luna C18 150*25 mm*10 um; mobile phase: [water (FA)-ACN]; B %: 25%-55%, 10 min) and lyophilized to afford N-(2-(2,6-dioxopiperidin-3-yl)-1-oxoisoindolin-5-yl)-4-(1-methoxyethyl)indoline-1-carboxamide (45.82 mg, 95.1 umol, 18% yield, 96% purity) as a white solid. ¹H NMR (400 MHz, DMSO-d₆) δ=11.01-10.93 (m, 1H), 8.85 (s, 1H), 7.92 (s, 1H), 7.81 (d, J=7.6 Hz, 1H), 7.65 (s, 2H), 7.16 (t, J=8.0 Hz, 1H), 6.88 (d, J=7.6 Hz, 1H), 5.09 (dd, J=5.0, 13.2 Hz, 1H), 4.47-4.40 (m, 1H), 4.37 (q, J=6.4 Hz, 1H), 4.32-4.26 (m, 1H), 4.19 (br t, J=9.6 Hz, 2H), 3.21-3.16 (m, 2H), 3.14 (s, 3H), 2.95-2.87 (m, 1H), 2.60 (br dd, J=2.0, 15.2 Hz, 1H), 2.40 (br d, J=4.4 Hz, 1H), 2.05-1.96 (m, 1H), 1.33 (d, J=6.4 Hz, 3H). MS (ESI) m/z. 463.1 [M+H]⁺

### Example 112. Synthesis of Compound 318

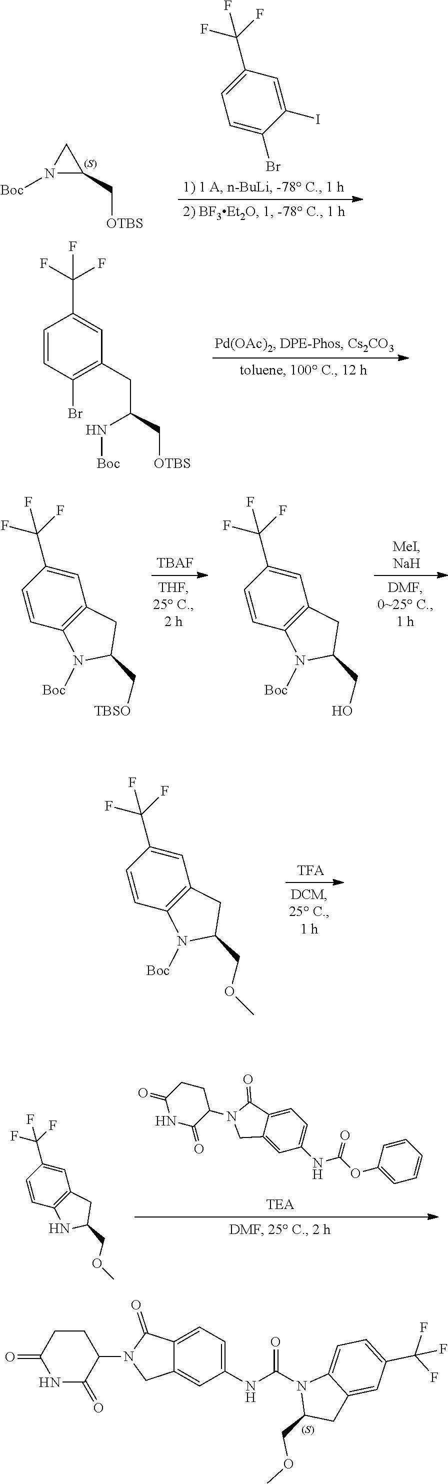

Step 1. To a solution of 1-bromo-2-iodo-4-(trifluoromethyl)benzene (600 mg, 1.71 mmol, 2.73 eq) in toluene (8.00 mL) was added n-butyllithium (2.5 M, 300 μL, 1.20 eq) at −78° C., the mixture was stirred at −78° C. for 1 h, then tert-butyl (S)-(1-((tert-butyldimethylsilyl)oxy)-3-hydroxypropan-2-yl)carbamate (180 mg, 626 mol, 1.00 eq) in toluene (1.00 mL) and boron trifluoride diethyl etherate (133 mg, 939 mol, 115 μL, 1.50 eq) was added, the mixture was stirred at −78° C. for another 1 h. The reaction mixture was quenched with saturated sodium bicarbonate (10 mL) and poured into water (100 mL), then extracted with ethyl acetate (3×80 mL). The combined organic phase was washed with brine (100 mL), dried over anhydrous sodium sulfate, filtered and concentrated in vacuum to give a residue. The residue was purified by reversed phase (0.1% FA condition) to afford (S)-tert-butyl (1-(2-bromo-5-(trifluoromethyl)phenyl)-3-((tert-butyldimethylsilyl) oxy)propan-2-yl)carbamate (60.0 mg, 117 mol, 9% yield) as a white solid. ¹H NMR (400 MHz, DMSO-d₆) δ=7.75 (d, J=8.4 Hz, 1H), 7.60 (s, 1H), 7.48-7.41 (m, 1H), 6.71 (d, J=9.5 Hz, 1H), 3.81-3.67 (m, 1H), 3.53-3.47 (m, 2H), 3.08-3.03 (m, 1H), 2.62-2.55 (m, 1H), 1.17 (s, 9H), 0.82 (s, 9H), 0.00 (s, 6H).

Step 2. To a solution of (S)-tert-butyl (1-(2-bromo-5-(trifluoromethyl)phenyl)-3-((tert-butyldimethylsilyl) oxy)propan-2-yl)carbamate (120 mg, 234 mol, 1.00 eq), [2-(2-diphenylphosphanylphenoxy)phenyl]-diphenyl-phosphane (25.0 mg, 46.8 mol, 0.200 eq) and cesium carbonate (152 mg, 468 mol, 2.00 eq) in toluene (3.00 mL) was added palladium acetate (5.26 mg, 23.4 mol, 0.100 eq), the mixture was stirred at 100° C. for 12 h under nitrogen atmosphere. The reaction mixture was poured into water (60 mL) and extracted with ethyl acetate (3×30 mL). The combined organic phase was washed with brine (100 mL), dried over anhydrous sodium sulfate, filtered and concentrated in vacuum to give a residue. The residue was purified by silica gel chromatography (Petroleum ether/Ethyl acetate=1/0 to 20/1) to afford (S)-tert-butyl 2-(((tert-butyldimethylsilyl)oxy)methyl)-5-(trifluoromethyl)indoline-1-carboxylate (80.0 mg, 185 mol, 79% yield) as yellow oil. ¹H NMR (400 MHz, DMSO-d₆) δ=7.93-7.67 (m, 1H), 7.56-7.47 (m, 2H), 4.56-4.44 (m, 1H), 3.91-3.81 (m, 1H), 3.71 (br d, J=9.9 Hz, 1H), 3.44-3.38 (m, 1H), 3.04 (br d, J=16.0 Hz, 1H), 1.56 (s, 9H), 0.67 (s, 9H), 0.02-0.02 (m, 3H), −0.11 (s, 3H).

Step 3. To a solution of (S)-tert-butyl 2-(((tert-butyldimethylsilyl)oxy)methyl)-5-(trifluoromethyl)indoline-1-carboxylate (80.0 mg, 185 mol, 1.00 eq) in tetrahydrofuran (2.00 mL) was added tetrabutylammonium fluoride (1.00 M, 222 μL, 1.20 eq), the mixture was stirred at 25° C. for 1 h. The reaction mixture was poured into water (50 mL) and extracted with ethyl acetate (3×30 mL). The combined organic phase was washed with brine (80 mL), dried over anhydrous sodium sulfate, filtered and concentrated in vacuum to give (S)-tert-butyl 2-(hydroxymethyl)-5-(trifluoromethyl)indoline-1-carboxylate (70.0 mg, crude) as yellow oil. MS (ESI) m/z 262.3 [M+H−56]⁺

Step 4. To a solution of (S)-tert-butyl 2-(hydroxymethyl)-5-(trifluoromethyl)indoline-1-carboxylate (70.0 mg, 220 mol, 1.00 eq) in dimethylformamide (2.00 mL) was added sodium hydride (13.2 mg, 330 mol, 60% purity, 1.50 eq) at 0° C., the mixture was stirred at 25° C. for 0.5 h, then iodomethane (62.6 mg, 441 mol, 27.0 μL, 2.00 eq) was added, the mixture was stirred at 25° C. for another 1.5 h. The reaction mixture was poured into water (40.0 mL) and ethyl acetate (3×20 mL). The combined organic phase was washed with brine (60 mL), dried over anhydrous sodium sulfate, filtered and concentrated in vacuum to give a residue. The residue was purified by silica gel chromatography (Petroleum ether/Ethyl acetate=1/0 to 20/1) to afford (S)-tert-butyl 2-(methoxymethyl)-5-(trifluoromethyl)indoline-1-carboxylate (60.0 mg, 181 mol, 82% yield) as colorless oil. ¹H NMR (400 MHz, DMSO-d₆) δ=7.74 (br s, 1H), 7.63-7.51 (m, 2H), 4.65-4.55 (m, 1H), 3.53-3.49 (m, 2H), 3.33 (br d, J=7.9 Hz, 1H), 3.28 (s, 3H), 3.02 (br d, J=16.8 Hz, 1H), 1.56 (s, 9H).

Step 5. To a solution of (S)-tert-butyl 2-(methoxymethyl)-5-(trifluoromethyl)indoline-1-carboxylate (60.0 mg, 181 mol, 1.00 eq) in dichloromethane (2.00 mL) was added trifluoroacetic acid (526 mg, 4.62 mmol, 400 μL, 25.5 eq), the mixture was stirred at 25° C. for 1 h. The reaction mixture was concentrated to give (S)-2-(methoxymethyl)-5-(trifluoromethyl)indoline (40.0 mg, crude) as yellow oil.

Step 6. To a solution of (S)-2-(methoxymethyl)-5-(trifluoromethyl)indoline (40.0 mg, 173 mol, 1.00 eq) and triethylamine (70.0 mg, 692 mol, 96.3 μL, 4.00 eq) in dimethylformamide (2.00 mL) was added phenyl (2-(2,6-dioxopiperidin-3-yl)-1-oxoisoindolin-5-yl)carbamate (59.0 mg, 155 mol, 0.900 eq), the mixture was stirred at 50° C. for 2 h, the reaction mixture was filtered to give a filtrate, the filtrate purified by Prep-HPLC (column: Phenomenex luna C18 150*25 mm*10 um; mobile phase: [water (FA)-ACN]; gradient:39%-69% B over 10 min) and lyophilized to afford (2S)—N-(2-(2,6-dioxopiperidin-3-yl)-1-oxoisoindolin-5-yl)-2-(methoxymethyl)-5-(trifluoromethyl)indoline-1-carboxamide (3.76 mg, 7.28 mol, 4% yield) as a white solid. ¹H NMR (400 MHz, DMSO-d₆) δ=10.99 (br s, 1H), 9.30 (s, 1H), 7.92 (d, J=8.5 Hz, 1H), 7.84 (s, 1H), 7.68 (d, J=8.4 Hz, 1H), 7.62-7.47 (m, 3H), 5.15-4.98 (m, 2H), 4.52-4.40 (m, 1H), 4.37-4.25 (m, 1H), 3.52 (br d, J=5.5 Hz, 2H), 3.49-3.46 (m, 1H), 3.33 (s, 3H), 3.00-2.87 (m, 2H), 2.61 (br d, J=16.5 Hz, 1H), 2.39 (dq, J=4.4, 13.3 Hz, 1H), 2.06-1.96 (m, 1H). MS (ESI) m/z 517.0 [M+H]⁺

### Example 113. Synthesis of Compound 329

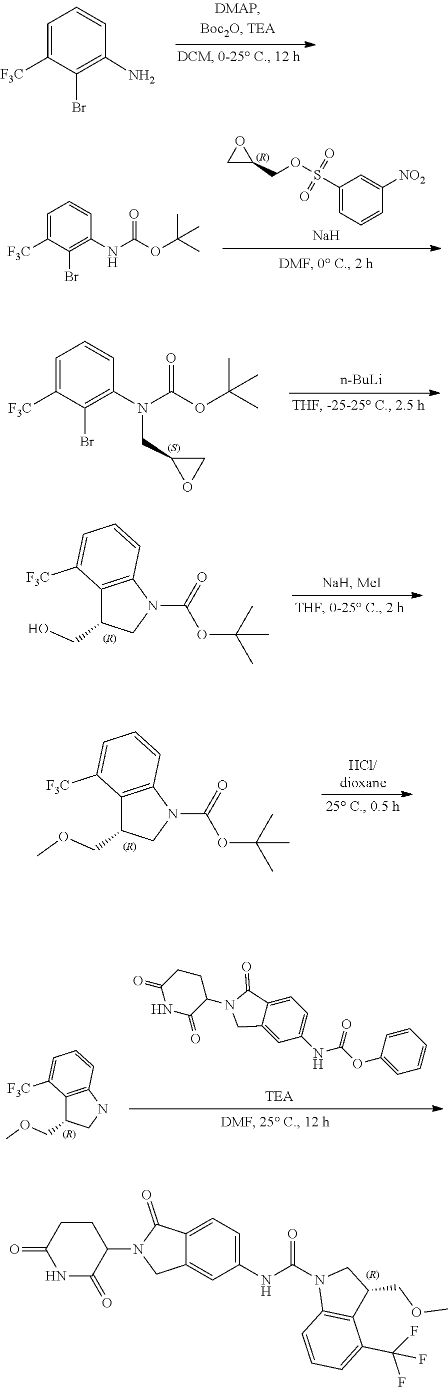

Step 1. To a solution of 2-bromo-3-(trifluoromethyl)aniline (5.00 g, 20.8 mmol, 1.00 eq), di-tert-butyl dicarbonate (4.55 g, 20.8 mmol, 4.79 mL, 1.00 eq) in dichloromethane (30.0 mL) was added triethylamine (3.16 g, 31.2 mmol, 4.35 mL, 1.50 eq) and 4-dimethylaminopyridin (255 mg, 2.08 mmol, 0.100 eq) at 0° C. The mixture was stirred at 25° C. for 12 h. The mixture was diluted with water (100 mL) and extracted with dichloromethane (3×150 mL). The organic phase was separated, washed with brine (100 mL), dried over sodium sulfate, filtered and concentrated under reduced pressure to get a residue. The residue was purified by column chromatography (SiO₂, Petroleum ether/Ethyl acetate=1/0) to get tert-butyl (2-bromo-3-(trifluoromethyl)phenyl)carbamate (4.50 g, 13.2 mmol, 64% yield) as yellow liquid. ¹H NMR (400 MHz, DMSO-d₆) δ=8.86 (s, 1H), 7.78 (d, J=7.9 Hz, 1H), 7.66-7.60 (m, 1H), 7.58-7.52 (m, 1H), 1.46 (s, 9H). MS (ESI) m/z 283.9 [M+H−56]⁺

Step 2. To a solution of tert-butyl (2-bromo-3-(trifluoromethyl)phenyl)carbamate (2.00 g, 5.88 mmol, 1.00 eq) in dry N,N-dimethyl formamide (30.0 mL) was added sodium hydride (353 mg, 8.82 mmol, 60% purity, 1.50 eq) and (R)-oxiran-2-ylmethyl 3-nitrobenzenesulfonate (1.98 g, 7.64 mmol, 1.30 eq) at 0° C. under nitrogen atmosphere. The mixture was stirred at 0° C. for 2 h. The mixture was quenched with saturated ammonium chloride solution (30 mL) and extracted with ethyl acetate (3×50 mL). The combined organic layers were washed with brine (50 mL), dried over anhydrous sodium sulfate, filtered and concentrated under reduced pressure to give a residue. The residue was purified by reversed-phase HPLC (column: spherical C18, 20-45 um, 100 Å, SW 120, mobile phase: [water (0.1% Formic Acid)-acetonitrile]) and lyophilized to give tert-butyl (S)-(2-bromo-3-(trifluoromethyl)phenyl)(oxiran-2-ylmethyl)carbamate (2.00 g, 4.95 mmol, 84% yield) as yellow oil. MS (ESI) m/z 339.8 [M+H−56]⁺

Step 3. To a solution of tert-butyl (S)-(2-bromo-3-(trifluoromethyl)phenyl)(oxiran-2-ylmethyl)carbamate (1.60 g, 4.04 mmol, 1.00 eq) in tetrahydrofuran (40.0 mL) was added n-butyllithium (2.5 M, 1.94 mL, 1.20 eq) dropwise at-25° C. under nitrogen atmosphere, while the temperature was kept at −25° C. to −20° C. The mixture was stirred at 25° C. for 2 h. The mixture was quenched with saturated ammonium chloride solution (30 mL) and extracted with ethyl acetate (3×30 mL). The organic phase was separated, washed with brine (30 mL), dried over anhydrous sodium sulfate, filtered and concentrated under reduced pressure to give a residue. The residue was purified by column chromatography (SiO₂, Petroleum ether/Ethyl acetate=200/1 to 20/1) to get tert-butyl (R)-3-(hydroxymethyl)-4-(trifluoromethyl)indoline-1-carboxylate (690 mg, 2.17 mmol, 54% yield) as a yellow solid. ¹H NMR (400 MHz, DMSO-d₆) δ=7.94 (br s, 1H), 7.41 (t, J=8.0 Hz, 1H), 7.24 (d, J=7.9 Hz, 1H), 5.09 (t, J=5.3 Hz, 1H), 4.09 (br d, J=11.3 Hz, 1H), 3.90 (br t, J=9.7 Hz, 1H), 3.63-3.46 (m, 2H), 3.28-3.19 (m, 1H), 1.52 (s, 9H). MS (ESI) m/z 261.9 [M+H−56]⁺

Step 4. To a solution of tert-butyl (R)-3-(hydroxymethyl)-4-(trifluoromethyl)indoline-1-carboxylate (690 mg, 2.17 mmol, 1.00 eq) in tetrahydrofuran (10.0 mL) was added sodium hydride (130 mg, 3.26 mmol, 60% purity, 1.50 eq) at 0° C. After stirring for 0.5 h, methyl iodide (617 mg, 4.35 mmol, 271 μL, 2.00 eq) was added to the reaction mixture. The mixture was stirred at 25° C. for 2 h. The mixture was quenched with saturated ammonium chloride solution (30 mL) and extracted with ethyl acetate (3×30 mL). The organic phase was separated, washed with brine (30 mL), dried over anhydrous sodium sulfate, filtered and concentrated under reduced pressure to give a residue. The residue was purified by column chromatography (SiO₂, Petroleum ether/Ethyl acetate=1/0) to get tert-butyl (R)-3-(methoxymethyl)-4-(trifluoromethyl)indoline-1-carboxylate (600 mg, 1.81 mmol, 83% yield) as colorless oil. ¹H NMR (400 MHz, CDCl₃) δ=8.46-7.81 (m, 1H), 7.35-7.28 (m, 1H), 7.20 (d, J=7.9 Hz, 1H), 4.20-4.10 (m, 1H), 3.90 (dd, J=8.7, 11.1 Hz, 1H), 3.74 (br t, J=9.0 Hz, 1H), 3.54 (dd, J=3.6, 9.5 Hz, 1H), 3.37 (s, 3H), 3.34-3.27 (m, 1H), 1.57 (s, 9H).

Step 5. To a solution of tert-butyl (R)-3-(methoxymethyl)-4-(trifluoromethyl)indoline-1-carboxylate (100 mg, 302 umol, 1.00 eq) in hydrochloric acid/dioxane (4 M, 1 mL) was stirred at 25° C. for 0.5 h. The mixture was concentrated under reduced pressure to get (R)-3-(methoxymethyl)-4-(trifluoromethyl)indoline (80 mg, crude) as a yellow solid. MS (ESI) m/z 233.1 [M+H]⁺

Step 6. To a solution of (R)-3-(methoxymethyl)-4-(trifluoromethyl)indoline (50.0 mg, 187 umol, 1.00 eq, HCl), phenyl (2-(2,6-dioxopiperidin-3-yl)-1-oxoisoindolin-5-yl)carbamate (70.8 mg, 187 umol, 1.00 eq) in N,N-dimethyl formamide (0.500 mL) was added triethylamine (56.7 mg, 560 umol, 78.0 uL, 3.00 eq). The mixture was stirred at 25° C. for 12 h. The mixture was stirred at 50° C. for 2 h. The mixture was adjusted to pH=5-6 with formic acid and filtered. The filtrate was purified by reversed phase HPLC (column: spherical C 18, 20-45 um, 100 Å, SW 40, mobile phase: [water (0.1% Formic Acid)-acetonitrile) and lyophilized to afford (3R)—N-(2-(2,6-dioxopiperidin-3-yl)-1-oxoisoindolin-5-yl)-3-(methoxymethyl)-4-(trifluoromethyl)indoline-1-carboxamide (40.2 mg, 77.1 umol, 41% yield, 99% purity) as a white solid. ¹H NMR (400 MHz, DMSO-d₆) δ=10.98 (s, 1H), 9.07 (s, 1H), 8.22 (d, J=8.3 Hz, 1H), 7.94 (s, 1H), 7.73-7.68 (m, 1H), 7.68-7.63 (m, 1H), 7.44 (t, J=8.1 Hz, 1H), 7.27 (d, J=7.8 Hz, 1H), 5.10 (dd, J=5.1, 13.3 Hz, 1H), 4.48-4.40 (m, 1H), 4.35-4.27 (m, 2H), 4.18 (t, J=9.4 Hz, 1H), 3.91-3.83 (m, 1H), 3.49-3.44 (m, 1H), 3.40-3.34 (m, 1H), 3.29 (s, 3H), 2.97-2.86 (m, 1H), 2.64-2.56 (m, 1H), 2.44-2.37 (m, 1H), 2.05-1.96 (m, 1H). MS (ESI) m/z 517.2 [M+H]⁺

### Example 114. Synthesis of Compound 319

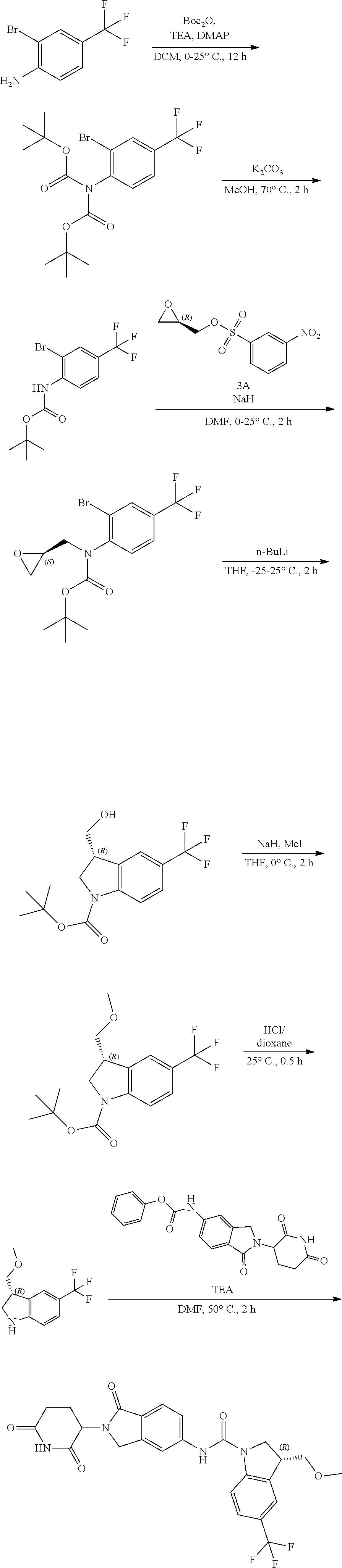

Step 1. To a solution of 2-bromo-4-(trifluoromethyl)aniline (5.00 g, 20.8 mmol, 1.00 eq) in dichloromethane (50.0 mL) was added triethylamine (4.22 g, 41.6 mmol, 5.80 mL, 2.00 eq), di-tert-butyl dicarbonate (9.09 g, 41.6 mmol, 9.57 mL, 2.00 eq) and 4-dimethylaminopyridin (254 mg, 2.08 mmol, 0.100 eq) at 0° C. Then the reaction mixture was stirred at 25° C. for 12 h. The reaction mixture was diluted with water (200 mL) and extracted with dichloromethane (3×100 mL). The combined organic layers were washed with brine (3×20 mL), dried over anhydrous sodium sulfate, filtered and concentrated under reduced pressure to give tert-butyl N-2-bromo-4-(trifluoromethyl)phenyl-N-tert-butoxycarbonyl-carbamate (9.00 g, crude) as a yellow solid. MS (ESI) m/z. 903.0 [2M+Na]⁺

Step 2. To a solution of tert-butyl N-2-bromo-4-(trifluoromethyl)phenyl-N-tert-butoxycarbonyl-carbamate (9.00 g, 20.4 mmol, 1.00 eq) in methanol (90.0 mL) was added potassium carbonate (5.65 g, 40.8 mmol, 2.00 eq). Then the reaction mixture was stirred at 70° C. for 2 h. The reaction mixture was filtered. The filtrate was concentrated in vacuum. The residue was purified by column chromatography (SiO₂, Petroleum ether/Ethyl acetate=1/0) and concentrated in vacuum to give tert-butyl (2-bromo-4-(trifluoromethyl)phenyl)carbamate (6.00 g, 16.7 mmol, 81% yield, 95% purity) as a white solid. ¹H NMR (400 MHz, DMSO-d₆) δ=8.77 (s, 1H), 8.00 (d, J=1.6 Hz, 1H), 7.92-7.79 (m, 1H), 7.73 (dd, J=1.6, 8.8 Hz, 1H), 1.49 (s, 9H).

Step 3. To a solution of tert-butyl (2-bromo-4-(trifluoromethyl)phenyl)carbamate (2.00 g, 5.88 mmol, 1.00 eq) in dry N,N-dimethyl formamide (30.0 mL) was added sodium hydride (353 mg, 8.82 mmol, 60% purity, 1.50 eq) and (R)-oxiran-2-ylmethyl 3-nitrobenzenesulfonate (1.98 g, 7.64 mmol, 1.30 eq) at 0° C. under nitrogen atmosphere. The mixture was stirred at 0° C. for 2 h. The mixture was quenched with saturated ammonium chloride solution (50 mL) and extracted with ethyl acetate (3×80 mL). The combined organic layers were washed with brine (50 mL), dried over anhydrous sodium sulfate, filtered and concentrated under reduced pressure to give a residue. The residue was purified by column chromatography (SiO₂, Petroleum ether/Ethyl acetate=100/1 to 30/1) and concentrated in vacuum to give tert-butyl (S)-(2-bromo-4-(trifluoromethyl)phenyl)(oxiran-2-ylmethyl)carbamate (1.80 g, 4.54 mmol, 77% yield) as yellow oil. MS (ESI) m/z 339.8 [M+H−56]⁺

Step 4. To a solution of tert-butyl (S)-(2-bromo-4-(trifluoromethyl)phenyl)(oxiran-2-ylmethyl)carbamate (1.30 g, 3.28 mmol, 1.00 eq) in tetrahydrofuran (30.0 mL) was added n-butyllithium (2.5 M, 1.97 mL, 1.20 eq) dropwise at-25° C. under nitrogen atmosphere, while the temperature was kept at −25° C. to −20° C. The mixture was stirred at 25° C. for 2 h. The mixture was quenched with saturated ammonium chloride solution (30 mL) and extracted with ethyl acetate (3×30 mL). The organic phase was separated, washed with brine (30 mL), dried over anhydrous sodium sulfate, filtered and concentrated under reduced pressure to give a residue. The residue was purified by column chromatography (SiO₂, Petroleum ether/Ethyl acetate=200/1 to 10/1) to get tert-butyl (R)-3-(hydroxymethyl)-5-(trifluoromethyl)indoline-1-carboxylate (400 mg, 1.21 mmol, 37% yield, 96% purity) as yellow oil. ¹H NMR (400 MHz, DMSO-d₆) δ=8.01-7.63 (m, 1H), 7.59 (s, 1H), 7.53 (br d, J=8.3 Hz, 1H), 4.99 (t, J=5.2 Hz, 1H), 4.08-3.98 (m, 1H), 3.81 (dd, J=5.6, 11.2 Hz, 1H), 3.66-3.59 (m, 1H), 3.54 (td, J=5.6, 10.8 Hz, 1H), 3.51-3.43 (m, 1H), 1.52 (s, 9H). MS (ESI) m/z 261.9 [M+H−56]⁺

Step 5. To a solution of tert-butyl (R)-3-(hydroxymethyl)-5-(trifluoromethyl)indoline-1-carboxylate (350 mg, 1.10 mmol, 1.00 eq) in tetrahydrofuran (10.0 mL) was added sodium hydride (66.2 mg, 1.65 mmol, 60% purity, 1.50 eq) at 0° C. After stirring for 0.5 h, methyl iodide (313 mg, 2.21 mmol, 137 μL, 2.00 eq) was added to the reaction mixture. The mixture was stirred at 25° C. for 2 h. The mixture was quenched with saturated ammonium chloride solution (10 mL) and extracted with ethyl acetate (3×30 mL). The organic phase was separated, washed with brine (10 mL), dried over anhydrous sodium sulfate, filtered and concentrated under reduced pressure to give a residue. The residue was purified by column chromatography (SiO₂, Petroleum ether/Ethyl acetate=200/1 to 20/1) to get tert-butyl (R)-3-(methoxymethyl)-5-(trifluoromethyl)indoline-1-carboxylate (230 mg, 694 mol, 63% yield) as yellow oil. ¹H NMR (400 MHz, CDCl₃) δ=8.17-7.73 (m, 1H), 7.48 (br d, J=8.4 Hz, 1H), 7.44 (s, 1H), 4.19-4.06 (m, 1H), 3.92-3.81 (m, 1H), 3.67-3.56 (m, 2H), 3.52-3.45 (m, 1H), 3.42 (s, 3H), 1.59 (s, 9H).

Step 6. To a solution of tert-butyl (R)-3-(methoxymethyl)-5-(trifluoromethyl)indoline-1-carboxylate (100 mg, 302 umol, 1.00 eq) in hydrochloric acid/dioxane (4 M, 2.00 mL) was stirred at 25° C. for 0.5 h. The mixture was concentrated under reduced pressure to get (R)-3-(methoxymethyl)-5-(trifluoromethyl)indoline (70.0 mg, crude) as a yellow solid. MS (ESI) m/z 232.1 [M+H]⁺

Step 7. To a solution of (R)-3-(methoxymethyl)-5-(trifluoromethyl)indoline (70.0 mg, 261 mol, 1.00 eq, HCl), phenyl (2-(2,6-dioxopiperidin-3-yl)-1-oxoisoindolin-5-yl)carbamate (99.2 mg, 261 mol, 1.00 eq) in N,N-dimethyl formamide (2.00 mL) was added triethylamine (79.4 mg, 784 mol, 109 μL, 3.00 eq). The mixture was stirred at 50° C. for 2 h. The mixture was adjusted to pH=5-6 with formic acid and filtered. The filtrate was purified by reversed phase HPLC (column: spherical C 18, 20-45 um, 100 Å, SW 40, mobile phase: [water (0.1% formic acid)-acetonitrile) and lyophilized to get a crude product. The crude product was purified by Prep-HPLC (column: Phenomenex luna C18 150*25 mm*10 um; mobile phase: [water (formic acid)-acetonitrile]; gradient:33%-63% B over 9 min) and lyophilized to afford (3R)—N-(2-(2,6-dioxopiperidin-3-yl)-1-oxoisoindolin-5-yl)-3-(methoxymethyl)-5-(trifluoromethyl)indoline-1-carboxamide (32.5 mg, 62.4 mol, 24% yield, 99% purity) as a white solid.

¹H NMR (400 MHz, DMSO-d₆) δ=10.98 (br s, 1H), 9.03 (s, 1H), 8.04 (d, J=8.4 Hz, 1H), 7.94 (s, 1H), 7.71-7.64 (m, 2H), 7.62 (s, 1H), 7.55 (br d, J=8.4 Hz, 1H), 5.09 (dd, J=5.2, 13.2 Hz, 1H), 4.47-4.40 (m, 1H), 4.37-4.27 (m, 2H), 4.10 (dd, J=5.6, 10.0 Hz, 1H), 3.84-3.75 (m, 1H), 3.68 (dd, J=5.2, 9.2 Hz, 1H), 3.55-3.49 (m, 1H), 3.33-3.32 (m, 3H), 2.98-2.85 (m, 1H), 2.64-2.54 (m, 1H), 2.39 (br dd, J=4.4, 13.2 Hz, 1H), 2.05-1.95 (m, 1H).

¹H NMR (400 MHz, CDCl₃) δ=8.12 (d, J=8.8 Hz, 1H), 8.09-8.04 (m, 1H), 8.02 (br s, 1H), 7.81-7.73 (m, 1H), 7.53 (br d, J=8.4 Hz, 1H), 7.44 (s, 1H), 7.24 (br d, J=1.6 Hz, 1H), 6.87-6.82 (m, 1H), 5.24-5.14 (m, 1H), 4.52-4.44 (m, 1H), 4.38-4.31 (m, 1H), 4.30-4.22 (m, 1H), 4.13-4.06 (m, 1H), 3.86-3.77 (m, 1H), 3.73-3.63 (m, 1H), 3.55-3.48 (m, 1H), 3.45 (s, 3H), 2.98-2.90 (m, 1H), 2.89-2.78 (m, 1H), 2.47-2.32 (m, 1H), 2.28-2.18 (m, 1H). MS (ESI) m/z 517.2 [M+H]⁺

### Example 115. Synthesis of Compound 321

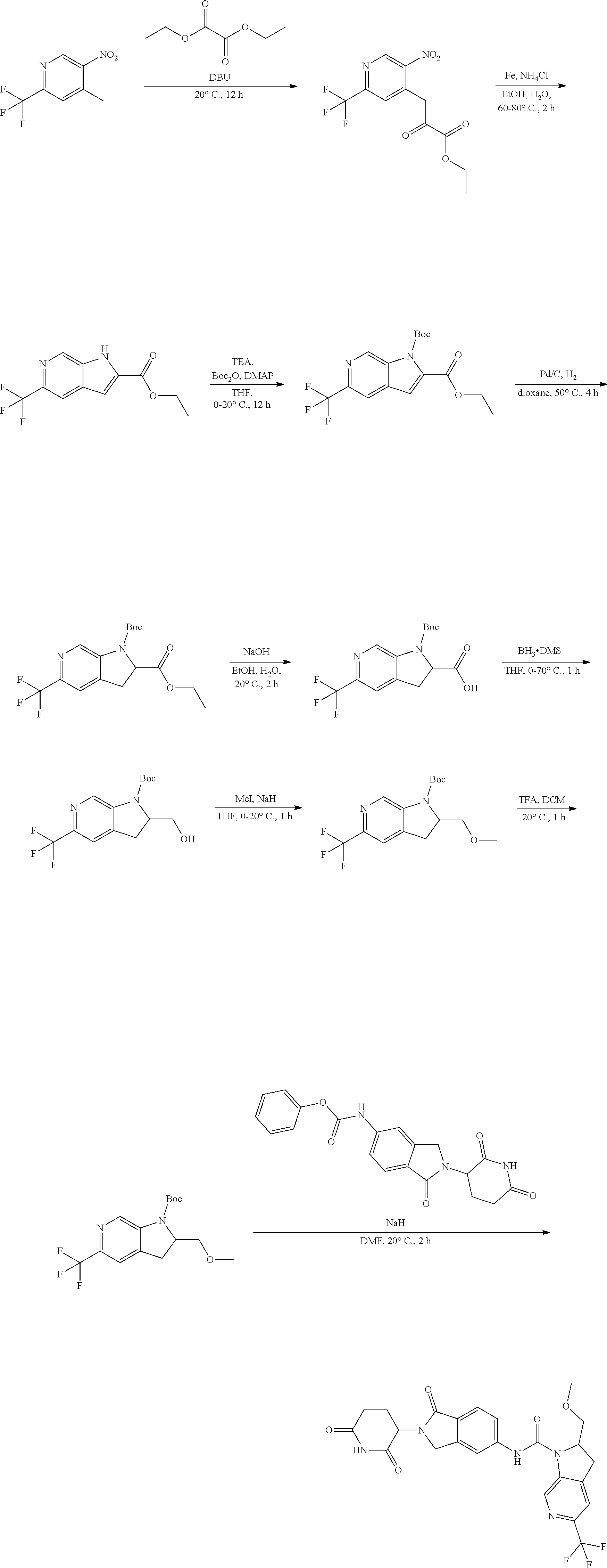

Step 1. To a solution of 4-methyl-5-nitro-2-(trifluoromethyl)pyridine (20.0 g, 97.0 mmol, 1.00 eq) in diethyl oxalate (66.6 g, 456 mmol, 62.2 mL, 4.70 eq) was added 1,8-diazabicyclo[5.4.0]undec-7-ene (35.4 g, 232 mmol, 35.1 mL, 2.40 eq). The reaction mixture was stirred at 20° C. for 12 h. The reaction mixture was diluted with water (1 L) and exacted with ethyl acetate (3×300 mL). The organic phase was separated, washed with brine (3×1 L), dried over sodium sulfate, filtered and concentrated under reduced pressure to afford ethyl 3-(5-nitro-2-(trifluoromethyl)pyridin-4-yl)-2-oxopropanoate (25.0 g, crude) as brown oil. MS (ESI) m/z 307.1 [M+H]⁺

Step 2. To a solution of ethyl 3-(5-nitro-2-(trifluoromethyl)pyridin-4-yl)-2-oxopropanoate (25.0 g, 81.6 mmol, 1.00 eq) in ethanol (200 mL) and water (50.0 mL) was added iron powder (22.8 g, 408 mmol, 5.00 eq) and ammonium chloride (21.8 g, 408 mmol, 5.00 eq) at 60° C. The reaction mixture was stirred at 80° C. for 2 h. The reaction mixture was filtered and concentrated to give a residue. The residue was purified by column chromatography (SiO₂, Petroleum ether/Ethyl acetate=10/1 to 5/1) to afford ethyl 5-(trifluoromethyl)-1H-pyrrolo[2,3-c]pyridine-2-carboxylate (8.40 g, 32.5 mmol, 39% yield) as a yellow solid. ¹H NMR (400 MHz, DMSO-d₆) δ=12.89 (br s, 1H), 8.95 (s, 1H), 8.20 (s, 1H), 7.33 (s, 1H), 4.39 (q, J=7.1 Hz, 2H), 1.36 (t, J=6.9 Hz, 3H). MS (ESI) m/z 259.1 [M+H]⁺

Step 3. To a solution of ethyl 5-(trifluoromethyl)-1H-pyrrolo[2,3-c]pyridine-2-carboxylate (8.40 g, 32.5 mmol, 1.00 eq), di-tert-butyl dicarbonate (21.3 g, 97.6 mmol, 22.4 mL, 3.00 eq), 4-dimethylaminopyridine (1.99 g, 16.2 mmol, 0.500 eq) and triethylamine (9.88 g, 97.6 mmol, 13.5 mL, 3.00 eq) in tetrahydrofuran (200 mL) was at 0° C. Then the reaction mixture was stirred at 20° C. for 12 h. The reaction mixture was concentrated under reduced pressure to give a residue. The residue was purified by column chromatography (SiO₂, Petroleum ether/Ethyl acetate=1/0 to 10/1) to afford 1-(tert-butyl) 2-ethyl 5-(trifluoromethyl)-1H-pyrrolo[2,3-c]pyridine-1,2-dicarboxylate (3.00 g, 8.37 mmol, 25% yield) as yellow oil. ¹H NMR (400 MHz, CDCl₃) δ=9.47 (s, 1H), 7.95 (s, 1H), 7.09 (s, 1H), 4.44 (q, J=7.2 Hz, 2H), 1.68 (s, 9H), 1.42 (t, J=7.2 Hz, 3H). MS (ESI) m/z 259.1 [M−100+H]⁺

Step 4. To a solution of 1-(tert-butyl) 2-ethyl 5-(trifluoromethyl)-1H-pyrrolo[2,3-c]pyridine-1,2-dicarboxylate (750 mg, 2.09 mmol, 1.00 eq) in dioxane (15.0 mL) was added palladium on carbon (2.00 g, 10% purity) under nitrogen atmosphere. Then the mixture was degassed and purged with hydrogen for 3 times, the reaction mixture was stirred at 50° C. for 4 h under hydrogen (50 psi) atmosphere. The reaction mixture was filtered and concentrated under reduced pressure to afford 1-(tert-butyl) 2-ethyl 5-(trifluoromethyl)-2,3-dihydro-1H-pyrrolo[2,3-c]pyridine-1,2-dicarboxylate (900 mg, 2.50 mmol, 59% yield) as brown oil. ¹H NMR (400 MHz, DMSO-d₆) δ=9.04-8.63 (m, 1H), 7.80 (s, 1H), 4.99 (dd, J=4.3, 11.6 Hz, 1H), 4.26-4.08 (m, 2H), 3.76-3.66 (m, 1H), 3.28-3.19 (m, 1H), 1.59-1.41 (m, 9H), 1.25-1.19 (m, 3H). MS (ESI) m/z 361.1 [M+H]⁺

Step 5. To a solution of 1-(tert-butyl) 2-ethyl 5-(trifluoromethyl)-2,3-dihydro-1H-pyrrolo[2,3-c]pyridine-1,2-dicarboxylate (900 mg, 2.50 mmol, 1.00 eq), sodium hydroxide (499 mg, 12.4 mmol, 5.00 eq) and water (5.00 mL) in ethyl alcohol (10.0 mL) was stirred at 20° C. for 2 h. The reaction mixture was adjusted to pH=6 with acetic acid, diluted with water (50 mL), extracted with ethyl acetate (3×50 mL). The combined organic phase was separated, washed with brine (20 mL), dried over anhydrous sodium sulfate, filtered and concentrated to afford 1-(tert-butoxycarbonyl)-5-(trifluoromethyl)-2,3-dihydro-1H-pyrrolo[2,3-c]pyridine-2-carboxylic acid (800 mg, 2.41 mmol, 96% yield) as yellow oil. ¹H NMR (400 MHz, DMSO-d₆) δ=12.18-11.97 (m, 1H), 9.00-8.63 (m, 1H), 7.78 (s, 1H), 4.88 (dd, J=4.0, 11.8 Hz, 1H), 3.74-3.60 (m, 1H), 3.22 (br d, J=18.3 Hz, 1H), 1.59-1.43 (m, 9H). MS (ESI) m/z 333.1 [M+H]⁺

Step 6. To a solution of 1-(tert-butoxycarbonyl)-5-(trifluoromethyl)-2,3-dihydro-1H-pyrrolo[2,3-c]pyridine-2-carboxylic acid (800 mg, 241 mmol, 1.00 eq) in tetrahydrofuran (10.0 mL) was added borane dimethyl sulfide complex (10 M, 722 μL, 3.00 eq) at 0° C. The reaction mixture was stirred at 70° C. for 1 h. The reaction mixture was quenched by addition methanol (20 mL) at 0° C., then concentrated to afford tert-butyl 2-(hydroxymethyl)-5-(trifluoromethyl)-2,3-dihydro-1H-pyrrolo[2,3-c]pyridine-1-carboxylate (760 mg, 2.39 mmol, 99% yield) as yellow oil. ¹H NMR (400 MHz, DMSO-d₆) δ=8.98-8.56 (m, 1H), 7.76 (s, 1H), 4.92 (br s, 1H), 4.48-4.41 (m, 1H), 3.66-3.58 (m, 1H), 3.55-3.47 (m, 1H), 3.42-3.36 (m, 1H), 3.10 (dd, J=2.8, 17.8 Hz, 1H), 1.53 (s, 9H). MS (ESI) m/z 319.2 [M+H]⁺

Step 7. To a solution of tert-butyl 2-(hydroxymethyl)-5-(trifluoromethyl)-2,3-dihydro-1H-pyrrolo[2,3-c]pyridine-1-carboxylate (760 mg, 2.39 mmol, 1.00 eq) in tetrahydrofuran (10.0 mL) was added sodium hydride (191 mg, 4.78 mmol, 60% purity, 2.00 eq) at 0° C. The reaction mixture was stirred at 0° C. for 0.5 h. Then methyl iodide (1.69 mg, 11.9 mmol, 743 μL, 5.00 eq) was added and the mixture was stirred at 20° C. for 0.5 h. The reaction mixture was poured into ammonium chloride (100 mL) and extracted with ethyl acetate (3×50 mL). The combined organic phase was separated, washed with brine (50 mL), dried over anhydrous sodium sulfate, filtered and concentrated to give a residue. The residue was purified by silica gel chromatography (Petroleum ether/Ethyl acetate=1/0 to 10/1) to afford tert-butyl 2-(methoxymethyl)-5-(trifluoromethyl)-2,3-dihydro-1H-pyrrolo[2,3-c]pyridine-1-carboxylate (270 mg, 812 umol, 34% yield) as yellow oil. ¹H NMR (400 MHz, DMSO-d₆) δ=8.80 (br dd, J=4.3, 8.0 Hz, 1H), 7.78 (s, 1H), 4.63-4.54 (m, 1H), 3.59-3.51 (m, 1H), 3.47 (br dd, J=2.9, 9.9 Hz, 1H), 3.43-3.38 (m, 1H), 3.23 (s, 3H), 3.05 (br dd, J=2.1, 17.6 Hz, 1H), 1.54 (s, 9H). MS (ESI) m/z 333.1 [M+H]⁺

Step 8. A solution of tert-butyl 2-(methoxymethyl)-5-(trifluoromethyl)-2,3-dihydro-1H-pyrrolo[2,3-c]pyridine-1-carboxylate (270 mg, 812 umol, 1.00 eq) in dichloromethane (2.00 mL) was added trifluoroacetic acid (0.400 mL), the mixture was stirred at 20° C. for 1 h. The reaction mixture was concentrated under reduced pressure to afford 2-(methoxymethyl)-5-(trifluoromethyl)-2,3-dihydro-1H-pyrrolo[2,3-c]pyridine (180 mg, crude) as brown oil. MS (ESI) m/z 233.1 [M+H]⁺

Step 9. To a solution of 2-(methoxymethyl)-5-(trifluoromethyl)-2,3-dihydro-1H-pyrrolo[2,3-c]pyridine (100 mg, 430 mol, 1.00 eq), phenyl (2-(2,6-dioxopiperidin-3-yl)-1-oxoisoindolin-5-yl)carbamate (98.0 mg, 258 mol, 0.600 eq) and sodium hydride (51.6 mg, 1.29 mmol, 60% purity, 3.00 eq) in dimethyl formamide (3.00 mL) at 20° C. The reaction mixture was stirred at 20° C. for 1 h. Then phenyl (2-(2,6-dioxopiperidin-3-yl)-1-oxoisoindolin-5-yl)carbamate (98.0 mg, 258 mol, 0.600 eq) and sodium hydride (51.6 mg, 1.29 mmol, 60% purity, 3.00 eq) was added and the mixture was stirred at 20° C. for 1 h. The reaction mixture was added formic acid (3.00 mL) and filtered to give a filtrate. The filtrate was purified by reversed-phase HPLC (column: spherical C18, 20-45 um, 100 Å, SW 40, mobile phase: [water (0.1% Formic Acid)-acetonitrile) and lyophilized to afford N-(2-(2,6-dioxopiperidin-3-yl)-1-oxoisoindolin-5-yl)-2-(methoxymethyl)-5-(trifluoromethyl)-2,3-dihydro-1H-pyrrolo[2,3-c]pyridine-1-carboxamide (52.19 mg, 91.6 umol, 21% yield, 99% purity, formate) as an off-white solid. ¹H NMR (400 MHz, DMSO-d₆) δ=11.05-10.93 (m, 1H), 9.40 (br s, 1H), 9.06 (s, 1H), 8.45 (br s, 0.8H), 7.89 (s, 1H), 7.82 (s, 1H), 7.72-7.67 (m, 1H), 7.61 (br d, J=8.3 Hz, 1H), 5.11 (br dd, J=5.1, 13.1 Hz, 2H), 4.51-4.41 (m, 1H), 4.38-4.27 (m, 1H), 3.62-3.49 (m, 3H), 3.31 (br s, 3H), 3.06 (br d, J=17.6 Hz, 1H), 2.98-2.88 (m, 1H), 2.62 (br d, J=16.4 Hz, 1H), 2.43-2.40 (m, 1H), 2.05-1.97 (m, 1H). MS (ESI) m/z 518.3 [M+H]⁺

### Example 116. Synthesis of Compound 334 and 337

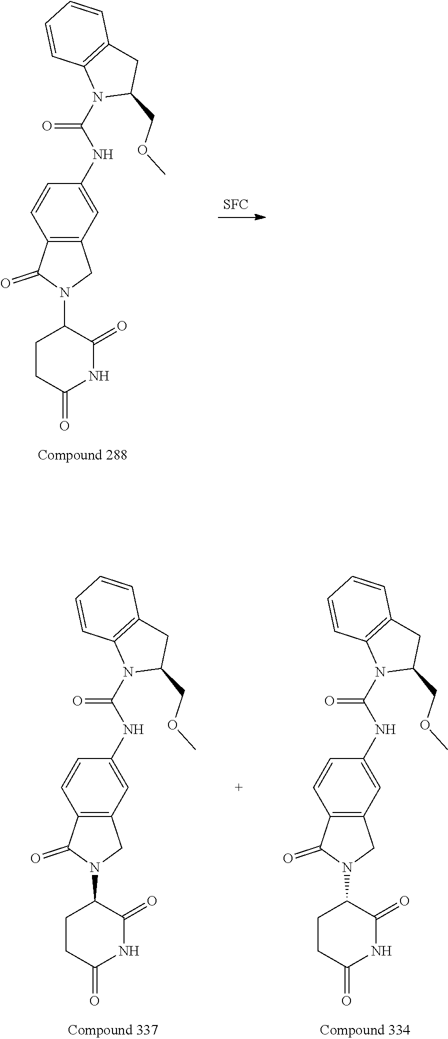

The compound (2S)—N-(2-(2,6-dioxopiperidin-3-yl)-1-oxoisoindolin-5-yl)-2-(methoxymethyl)indoline-1-carboxamide (300 mg, 668 umol,) was separated by supercritical fluid chromatography (column: Phenomenex-Cellulose-2 (250 mm*30 mm, 10 um); mobile phase: [propan-2-ol/acetonitrile]; B %: 60%-60%, 9 min) and concentrated to afford crude products. The product 1 was purified by Prep-HPLC (column: Waters Xbridge 150*25 mm*5 um; mobile phase: [water (ammonium hydrogen carbonate)-acetonitrile]; B %: 24%-54%, min) and lyophilized to afford (S)—N-(2-((R)-2,6-dioxopiperidin-3-yl)-1-oxoisoindolin-5-yl)-2-(methoxymethyl)indoline-1-carboxamide (74 mg, 163 umol, 29% yield, 99% purity) as a white solid. The product 2 was purified by Prep-HPLC (column: Waters Xbridge 150*25 mm*5 um; mobile phase: [water (ammonium hydrogen carbonate)-acetonitrile]; B %: 24%-54%, min) and lyophilized to afford (S)—N-(2-((S)-2,6-dioxopiperidin-3-yl)-1-oxoisoindolin-5-yl)-2-(methoxymethyl)indoline-1-carboxamide (82 mg, 181 umol, 33% yield, 99% purity) as a white solid.

¹H NMR (400 MHz, DMSO-d₆) δ=10.97 (br s, 1H), 9.16 (s, 1H), 7.82 (s, 1H), 7.76 (d, J=8.0 Hz, 1H), 7.65 (d, J=8.4 Hz, 1H), 7.53 (dd, J=1.6, 8.3 Hz, 1H), 7.22 (d, J=7.3 Hz, 1H), 7.15 (t, J=7.6 Hz, 1H), 7.03-6.90 (m, 1H), 5.09 (dd, J=5.1, 13.3 Hz, 1H), 5.02-4.85 (m, 1H), 4.48-4.38 (m, 1H), 4.37-4.17 (m, 1H), 3.52-3.44 (m, 2H), 3.42-3.36 (m, 1H), 3.34 (s, 3H), 2.97-2.87 (m, 1H), 2.83 (br d, J=16.3 Hz, 1H), 2.60 (br dd, J=2.1, 15.2 Hz, 1H), 2.45-2.35 (m, 1H), 2.07-1.89 (m, 1H). MS (ESI) m/z 449.4 [M+H]⁺

¹H NMR (400 MHz, DMSO-d₆) δ=10.97 (br s, 1H), 9.16 (s, 1H), 7.82 (s, 1H), 7.76 (d, J=8.0 Hz, 1H), 7.65 (d, J=8.4 Hz, 1H), 7.53 (dd, J=1.6, 8.3 Hz, 1H), 7.22 (d, J=7.4 Hz, 1H), 7.15 (t, J=7.9 Hz, 1H), 7.00-6.90 (m, 1H), 5.09 (dd, J=5.1, 13.2 Hz, 1H), 5.01-4.87 (m, 1H), 4.51-4.36 (m, 1H), 4.36-4.24 (m, 1H), 3.52-3.44 (m, 2H), 3.42-3.36 (m, 1H), 3.34 (s, 3H), 2.97-2.87 (m, 1H), 2.84 (br d, J=16.1 Hz, 1H), 2.62-2.58 (m, 1H), 2.44-2.36 (m, 1H), 2.06-1.96 (m, 1H). MS (ESI) m/z 449.2 [M+H]⁺

### Example 117. Synthesis of Compounds 359, 360, 361, and 362

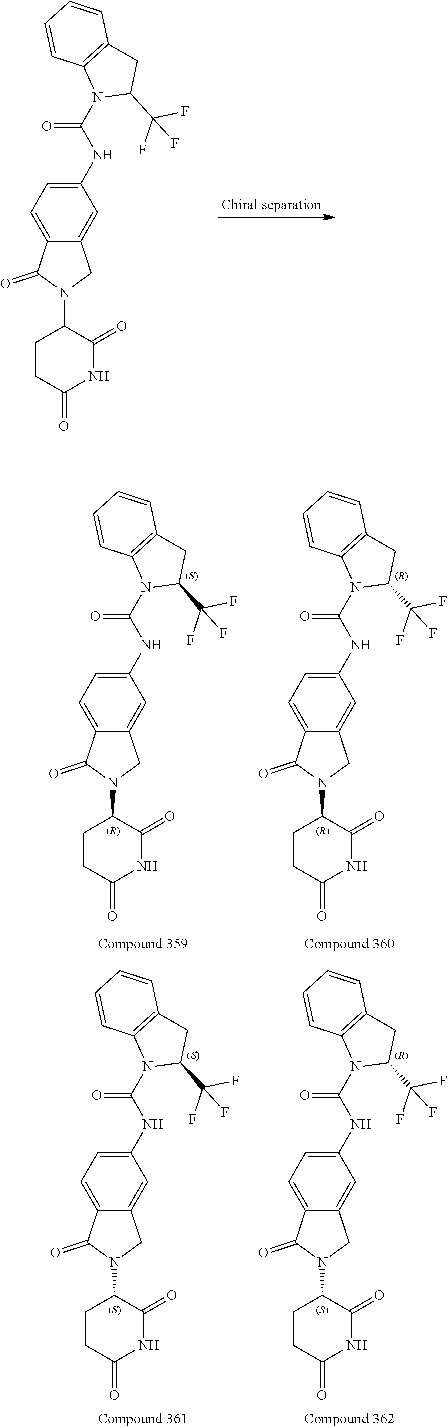

Step 1. The racemic material of N-(2-(2,6-dioxopiperidin-3-yl)-1-oxoisoindolin-5-yl)-2-(trifluoromethyl)indoline-1-carboxamide (100 mg, 211 umol, 13% yield) was purified by SFC (column: DAICEL CHIRALPAK AS (250 mm*30 mm, 10 um); mobile phase: [ACN/IPA (0.1% NH₃H₂O)]; B %: 50%-50%, A8.5; 60 min). The fractions were concentrated under reduced pressure to give Peak 1 (Compound 359), Peak 2 & Peak 3 (Compound 360 and compound 361) and Peak 4 (Compound 362).

The mixture of Peak 2 & Peak 3 (Compound 360 and compound 361) was separated by Chiral SFC (column: DAICEL CHIRALPAK IC (250 mm*30 mm, 10 um); mobile phase: [0.1% NH₃H2O IPA]; B %: 60%-60%, A2.7; 20 min).

Peak 1 (Compound 359) was purified by reversed-phase HPLC (column: spherical C18, 20-45 um, 100 Å, SW 40, mobile phase: [water-ACN]; B %: 5%-60%, 15 min). The desired fraction was collected and lyophilized to afford (S)—N-(2-((R)-2,6-dioxopiperidin-3-yl)-1-oxoisoindolin-5-yl)-2-(trifluoromethyl)indoline-1-carboxamide (11.5 mg, 24.1 umol, 11% yield, 99% purity) as a white solid. ¹H NMR (400 MHz, DMSO-d₆) δ=10.98 (s, 1H), 9.32 (s, 1H), 7.86 (s, 1H), 7.83 (d, J=8.1 Hz, 1H), 7.71-7.66 (m, 1H), 7.65-7.59 (m, 1H), 7.29 (d, J=7.1 Hz, 1H), 7.21 (t, J=7.7 Hz, 1H), 7.02 (t, J=7.4 Hz, 1H), 5.81-5.68 (m, 1H), 5.09 (dd, J=5.0, 13.4 Hz, 1H), 4.49-4.39 (m, 1H), 4.36-4.26 (m, 1H), 3.67 (br dd, J=9.8, 16.8 Hz, 1H), 3.17 (br d, J=17.1 Hz, 1H), 2.98-2.85 (m, 1H), 2.63-2.57 (m, 1H), 2.41-2.36 (m, 1H), 2.05-1.95 (m, 1H). MS (ESI) m/z 473.0 [M+H]⁺

Peak 2 (Compound 360) was purified by reversed-phase HPLC (column: spherical C18, 20-45 um, 100 Å, SW 40, mobile phase: [water-ACN]; B %: 5%-60%, 15 min). The desired fraction was collected and lyophilized to afford (R)—N-(2-((R)-2,6-dioxopiperidin-3-yl)-1-oxoisoindolin-5-yl)-2-(trifluoromethyl)indoline-1-carboxamide (7.57 mg, 15.3 umol, 7% yield, 96% purity) as a white solid. ¹H NMR (400 MHz, DMSO-d₆) δ=10.98 (br s, 1H), 9.32 (s, 1H), 7.87 (s, 1H), 7.83 (d, J=8.1 Hz, 1H), 7.71-7.66 (m, 1H), 7.65-7.57 (m, 1H), 7.29 (d, J=7.3 Hz, 1H), 7.21 (t, J=7.5 Hz, 1H), 7.02 (t, J=7.3 Hz, 1H), 5.80-5.69 (m, 1H), 5.09 (dd, J=5.1, 13.3 Hz, 1H), 4.50-4.39 (m, 1H), 4.38-4.27 (m, 1H), 3.67 (br dd, J=9.9, 16.9 Hz, 1H), 3.17 (br d, J=17.0 Hz, 1H), 2.98-2.85 (m, 1H), 2.63-2.57 (m, 1H), 2.39 (br dd, J=4.3, 12.9 Hz, 1H), 2.05-1.94 (m, 1H). MS (ESI) m/z 473.0 [M+H]⁺

Peak 3 (Compound 361) was purified by Prep-HPLC (column: Phenomenex C18 150*25 mm*10 um; mobile phase: [Water-ACN]; B %: 26%-56%, 9 min). The desired fraction was collected and lyophilized to afford (S)—N-(2-((S)-2,6-dioxopiperidin-3-yl)-1-oxoisoindolin-5-yl)-2-(trifluoromethyl)indoline-1-carboxamide (3.55 mg, 7.44 umol, 3% yield, 99% purity) as a white solid. ¹H NMR (400 MHz, DMSO-d₆) δ=10.98 (br s, 1H), 9.32 (s, 1H), 7.87 (s, 1H), 7.83 (d, J=8.1 Hz, 1H), 7.71-7.66 (m, 1H), 7.65-7.58 (m, 1H), 7.29 (d, J=7.4 Hz, 1H), 7.21 (t, J=8.1 Hz, 1H), 7.06-6.98 (m, 1H), 5.79-5.69 (m, 1H), 5.09 (dd, J=5.0, 13.1 Hz, 1H), 4.49-4.41 (m, 1H), 4.35-4.27 (m, 1H), 3.67 (dd, J=10.3, 16.9 Hz, 1H), 3.17 (br d, J=16.9 Hz, 1H), 2.98-2.83 (m, 1H), 2.60 (br dd, J=2.2, 15.9 Hz, 1H), 2.39 (br dd, J=4.3, 13.3 Hz, 1H), 2.07-1.94 (m, 1H). MS (ESI) m/z 473.0 [M+H]⁺

Peak 4 (Compound 362) was purified by reversed-phase HPLC (column: spherical C18, 20-45 um, 100 Å, SW 40, mobile phase: [water-ACN]; B %: 5%-60%, 15 min). The desired fraction was collected and lyophilized to afford (R)—N-(2-((S)-2,6-dioxopiperidin-3-yl)-1-oxoisoindolin-5-yl)-2-(trifluoromethyl)indoline-1-carboxamide (14.7 mg, 30.8 umol, 14% yield, 99% purity) as a white solid. ¹H NMR (400 MHz, DMSO-d₆) δ=10.98 (s, 1H), 9.32 (s, 1H), 7.86 (s, 1H), 7.83 (d, J=8.0 Hz, 1H), 7.70-7.66 (m, 1H), 7.64-7.60 (m, 1H), 7.29 (d, J=7.3 Hz, 1H), 7.21 (t, J=7.8 Hz, 1H), 7.06-6.99 (m, 1H), 5.80-5.69 (m, 1H), 5.09 (dd, J=5.1, 13.3 Hz, 1H), 4.49-4.41 (m, 1H), 4.34-4.27 (m, 1H), 3.67 (br dd, J=10.1, 16.6 Hz, 1H), 3.17 (br d, J=17.0 Hz, 1H), 2.98-2.86 (m, 1H), 2.63-2.58 (m, 1H), 2.39 (dd, J=4.4, 13.2 Hz, 1H), 2.04-1.96 (m, 1H). MS (ESI) m/z 473.0 [M+H]⁺

### Example 117. Synthesis of Compounds 113, 114, 116, 118-122, 125-134, 136, 138-141, 143-146, 150, 151, 153-161, 165-170, 172-177, 179-184, 188, 189, 193-196, 199, 200, 202, 204, 206-209, 211-13, 215, 217-282, and 290

Compounds 113, 114, 116, 118-122, 125-134, 136, 138-141, 143-146, 150, 151, 153-161, 165-170, 172-177, 179-184, 188, 189, 193-196, 199, 200, 202, 204, 206-209, 211-13, 215, 217-282, and 290 were prepared using methods analogous to the syntheses of other compounds disclosed herein.

### Example 118. CK1α NanoBit Assay

HEK293 clonal lines with CRISPR KI HiBiT tag on CSNK1A1 and stably expressing LgBiT protein were obtained from Promega (Madison, WI). Cells were plated at 5000 cells per well using Multiflo (BioTek) in 384-well white solid bottom plates (Corning, 3570BC) in 25 μl volume in DMEM media (DMEM, high glucose, HEPES, no phenol red (ThermoFisher Scientific, 21063029)) containing 10% FBS (Corning, 35-075-CV), 1% Penicillin/Streptomycin ((ThermoFisher Scientific, 15140-122), and 0.2% Endurazine (Nano-Glo Endurazine Live Cell Substrate (Promega, N2571)). Cells were incubated for 16 hours at 37° C., 5% CO₂. Depending on experiment 25 or 75 nL of a compound at 10 mM were added into the plate using an Echo®650 liquid handler (Labcyte) to achieve final concentration of 10 or 30 μM in wells. Cells were incubated at 37° C., 5% CO₂ for 24 hours and then signal was read on a Pherastar FSX using “LUM plus” optic module.

Data analysis was performed in Scinamic (Scinamic, Cambridge, MA). Luminescence response (R) was calculated by the formula: response=100*(S−N)/(P−N) where S is the signal of the well, N and P the mean negative and positive control values respectively of the same plate. The luminescence response was then fitted in Scinamic using a 3-parameter agonist logistic fit (hillslope=1, EC50>0, top/bottom unconstrained).

DC₅₀ data are reported in Table 2 for compounds in Table 1. In Table 2 below, According to the code, A represents a DC₅₀ value of ≤0.1 μM, B represents a DC₅₀ value>0.1 μM and ≤1 μM, C represents an DC₅₀ value>1 μM.

TABLE 2
CK1α data

| | CK1α DC₅₀ |
|---|---|
| Cpmd No. | (μM) 24 h |
| 101 | A |
| 102 | A |
| 103 | A |
| 104 | A |
| 105 | A |
| 106 | A |
| 107 | B |
| 108 | B |
| 109 | B |
| 110 | B |
| 111 | B |
| 112 | B |
| 113 | C |
| 114 | A |
| 115 | A |
| 116 | A |
| 117 | B |
| 118 | A |
| 119 | A |
| 120 | B |
| 121 | B |
| 122 | B |
| 123 | A |
| 124 | A |
| 125 | A |
| 126 | C |
| 127 | A |
| 128 | B |
| 129 | A |
| 130 | A |
| 131 | B |
| 132 | A |
| 133 | A |
| 134 | A |
| 135 | A |
| 136 | A |
| 137 | A |
| 138 | A |
| 139 | B |
| 140 | A |
| 141 | A |
| 142 | A |
| 143 | A |
| 144 | B |
| 145 | C |
| 146 | B |
| 147 | C |
| 148 | A |
| 149 | B |
| 150 | B |
| 151 | C |
| 152 | A |
| 153 | A |
| 154 | A |
| 155 | B |
| 156 | C |
| 157 | B |
| 158 | B |
| 159 | B |
| 160 | B |
| 161 | B |
| 162 | A |
| 163 | A |
| 164 | A |
| 165 | A |
| 166 | A |
| 167 | A |
| 168 | A |
| 169 | C |
| 170 | A |
| 171 | A |
| 172 | A |
| 173 | A |
| 174 | C |
| 175 | A |
| 176 | B |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | B |
| 196 | A |
| 197 | A |
| 198 | A |
| 199 | A |
| 200 | A |
| 201 | A |
| 202 | A |
| 203 | A |
| 204 | A |
| 205 | A |
| 206 | B |
| 207 | C |
| 208 | A |
| 209 | A |
| 210 | A |
| 211 | A |
| 212 | A |
| 213 | B |
| 214 | A |
| 215 | A |
| 216 | A |
| 217 | A |
| 218 | A |
| 219 | A |
| 220 | A |
| 221 | B |
| 222 | C |
| 223 | C |
| 224 | B |
| 225 | C |
| 226 | A |
| 227 | A |
| 228 | A |
| 229 | C |
| 230 | B |
| 231 | A |
| 232 | B |
| 233 | B |
| 234 | C |
| 235 | A |
| 236 | B |
| 237 | B |
| 238 | B |
| 239 | B |
| 240 | B |
| 241 | B |
| 242 | C |
| 243 | C |
| 244 | B |
| 245 | B |
| 246 | C |
| 247 | B |
| 248 | A |
| 249 | C |
| 250 | C |
| 251 | C |
| 252 | B |
| 253 | A |
| 254 | C |
| 255 | A |
| 256 | A |
| 257 | B |
| 258 | B |
| 259 | B |
| 260 | A |
| 261 | A |
| 262 | C |
| 263 | B |
| 264 | A |
| 265 | B |
| 266 | C |
| 267 | B |
| 268 | A |
| 269 | A |
| 270 | B |
| 271 | B |
| 272 | B |
| 273 | A |
| 274 | B |
| 275 | C |
| 276 | B |
| 277 | A |
| 278 | A |
| 279 | B |
| 280 | B |
| 281 | B |
| 282 | B |
| 283 | A |
| 284 | A |
| 285 | B |
| 286 | B |
| 287 | A |
| 288 | A |
| 289 | A |
| 290 | A |
| 291 | A |
| 292 | A |
| 293 | A |
| 294 | A |
| 295 | A |
| 296 | A |
| 297 | C |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | B |
| 311 | A |
| 312 | B |
| 313 | A |
| 314 | A |
| 315 | B |
| 316 | A |
| 317 | A |
| 318 | A |
| 319 | A |
| 320 | A |
| 321 | A |
| 322 | A |
| 323 | B |
| 324 | A |
| 325 | A |
| 326 | B |
| 327 | B |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | B |
| 348 | A |
| 349 | A |
| 350 | A |
| 351 | B |
| 352 | A |
| 353 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | B |
| 365 | B |

### Example 119. Western Blot Protocol

HEK293 cell line was purchased from ATCC (CRL-1573). HEK293 CRBN knock out (k/o) cell line (B2) was generated internally using CRISPR/Cas9 method and clonally expanded. Cells were plated at 2×10⁵ cells per well in 6-well tissue culture plates (VWR) in 2 ml of DMEM media (Gibco) containing 10% FBS (Gibco), and incubated for 16 hours at 37° C., 5% CO₂. Compounds were added to final concentration of 0.1 μM, 1 μM, 10 μM (DMSO concentration was kept constant at 0.1%), following incubation at 37° C., 5% CO₂ for additional 24 hours. Cell lysis was performed using RIPA buffer (Pierce) containing Halt™ Protease Inhibitor Cocktail (ThermoFisher Scientific). Lysates were boiled at 95° C. for 10 minutes and 12 μg of protein lysate per sample was resolved by SDS-PAGE using 12% gels (BioRad) and transferred to nitrocellulose membrane (BioRad). Membranes were blocked using LI-COR blocking buffer (LI-COR) at room temperature for 1 hour, followed by overnight incubation with rabbit anti-CK1α (Abcam ab206652), rabbit anti-CRBN (Sigma HPA045910) and mouse anti-α-tubulin (Sigma T9026) antibodies at 4° C. Secondary antibodies (LI-COR) were added for 1 hour at room temperature, followed by imaging with LI-COR imaging system Odyssey® CLx.

### Example 120. HTRF CRBN & Ternary Complex Assay

### HTRF Binding Assay

Binding of test compounds to CRBN/DDB1 was monitored in an HTRF assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. In this assay, compounds displace the fluorescently labeled thalidomide-based probe bound to CRBN and fluorescence is monitored with increasing compound concentration. Assays were conducted in Greiner white 384 well HiBase plates (Cat. No 784075-25) in 10 μL total volume. A one pot detection solution of CRBN/DDB1 (2.5 nM), anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Test compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM lenalidomide (Selleckchem Cat no. S1029) was transferred into the active-control wells for the assay and 10 nL of DMSO was transferred into the negative control wells. The assay was then allowed to incubate for 30 min at ambient temperature after transferring the test compounds. Plate measurements were taken on a Pherastar FSX (BMG Labtech, Germany) using the HTRF Red filter (Ex. 337 nm, em1: 620 nm, em2: 665 nm) (Flashes: 50, Integration time: 60-400 μs, Z-height: 10 mm, Ratio-multiplier: 10,000). Analysis, HTRF ratio and IC₅₀ values were derived using KNIME analytics (KNIME Zurich) transformation and fitting within Collaborative Drug Discovery (Collaborative Drug Discovery USA) using a 4-parameter logistic fit. HTRF ratio calculation was performed using the following formula:

$$HTRF Ratio = \frac{e {m}_{665 n m}}{e {m}_{620 n m}} * 10 , 000$$

where em₆₆₅ₙₘ represents the measured emission at 665 nm upon excitation at 337 nm and em₆₂₀ₙₘ the measured emission at 620 nm upon excitation at 337 nm. The 4 parameter logistic fitting model was performed using the following formula:

$$= {Y}_{Min} + \frac{( {Y}_{Max} - {Y}_{Min} )}{( 1 + {( \frac{{IC}_{50}}{X} )}^{H i l l S l o p e} )}$$

where Y represents the HTRF ratio response (as defined previously), X the compound concentration in μM, Yₘᵢₙ the minimum response plateau, Yₘₐₓ the maximum response plateau, IC₅₀ the concentration of agonist that gives a response half way between Yₘᵢₙ and Yₘₐₓ and HillSlope the steepness of the family of curves. No fitting model constraints was applied on Yₘᵢₙ, or Yₘₐₓ whilst IC₅₀>0 and −0.5>HillSlope>−3

### Cheng-Prusoff Transformation

Ki values were derived from the geometric mean of the IC₅₀ values using the Cheng-Prusoff transformation:

$${K}_{i} = \frac{{IC}_{50}}{1 + ( \frac{[ L ]}{{K}_{d}} )} = \frac{{IC}_{50}}{1 + ( \frac{0.1 uM}{0.1 \; 216 uM} )}$$

where [L] represents the concentration of fluorescent probe in μM, K_{d} the affinity (binding constant) of the fluorescent probe in units of μM and IC₅₀ the concentration of agonist that gives a response halfway between Yₘᵢₙ and Yₘₐₓ (as described in 1.2.1)

### Statistical Methods or Analyses

Analysis and IC₅₀ values were derived using KNIME analytics (KNIME, Zurich) transformation and fitting within Collaborative Drug Discovery (Collaborative Drug Discovery, USA) as described in 1.2.1. Ki values were derived from the geometric mean of the IC₅₀ values using the Cheng-Prusoff transformation as described in 2.2.2. Data was visualized in GraphPad Prism 8.1.2 (GraphPad, USA) and reported as mean and standard deviation. Microsoft Office Excel 2012 (Redmond, WA) was used for calculation of mean and standard deviation.

IC₅₀ data are reported in Table 3 for compounds in Table 1. In Table 3 below, According to the code, A represents a IC₅₀ value of ≤0.1 μM, B represents a IC₅₀ value>0.1 μM and ≤1 μM, C represents an IC₅₀ value>1 μM.

TABLE 3
HTRF CRBN & Ternary Complex data

| Cpmd No. | HTRF_Thali_Disp_DR_geo_IC₅₀ (μM) |
|---|---|
| 101 | B |
| 102 | B |
| 103 | B |
| 105 | B |
| 106 | B |
| 109 | B |
| 111 | B |
| 112 | C |
| 115 | B |
| 117 | B |
| 123 | B |
| 124 | B |
| 132 | B |
| 135 | B |
| 137 | B |
| 142 | B |
| 147 | B |
| 148 | A |
| 152 | B |
| 162 | B |
| 163 | B |
| 164 | B |
| 171 | B |
| 178 | B |
| 185 | B |
| 186 | B |
| 187 | B |
| 188 | B |
| 189 | B |
| 190 | B |
| 191 | B |
| 192 | B |
| 197 | C |
| 198 | B |
| 201 | C |
| 203 | B |
| 205 | B |
| 209 | B |
| 210 | B |
| 214 | B |
| 216 | B |
| 256 | B |
| 283 | B |
| 284 | B |
| 285 | B |
| 286 | B |
| 287 | B |
| 287 | B |
| 288 | B |
| 289 | B |
| 290 | B |
| 291 | B |
| 292 | B |
| 293 | B |
| 294 | B |
| 295 | B |
| 297 | B |
| 298 | B |
| 299 | B |
| 300 | B |
| 301 | B |
| 302 | B |
| 303 | B |
| 304 | B |
| 305 | B |
| 306 | B |
| 307 | C |
| 308 | B |
| 309 | B |
| 310 | B |
| 311 | B |
| 312 | B |
| 313 | B |
| 314 | B |
| 315 | B |
| 316 | B |
| 317 | B |
| 318 | B |
| 319 | B |
| 320 | B |
| 321 | B |
| 322 | B |
| 323 | B |
| 324 | B |
| 325 | B |
| 326 | B |
| 327 | B |
| 328 | B |
| 329 | B |
| 330 | B |
| 331 | B |
| 332 | B |
| 333 | B |
| 334 | C |
| 335 | B |
| 336 | B |
| 337 | B |
| 338 | B |
| 339 | B |
| 340 | B |
| 341 | B |
| 342 | B |
| 343 | B |
| 344 | B |
| 345 | A |
| 346 | A |
| 347 | B |
| 348 | B |
| 349 | B |
| 350 | B |
| 351 | B |
| 352 | B |
| 353 | B |
| 354 | B |
| 355 | A |
| 356 | B |
| 357 | C |
| 358 | B |
| 359 | C |
| 362 | B |
| 364 | B |
| 365 | B |

### Example 121. GI₅₀ Data

SKCO1, LS180, and LS174T cell lines were purchased from ATCC. CW2 cell line was purchased from Riken. GP2D cell line was purchased from Sigma/ECACC. All cell lines were cultured in manufacturer's recommended media at 37° C., 5% CO₂. Briefly, cells were suspended in 200 μL of media and seeded at 300 to 1,800 cells per well in tissue culture treated 96-well plates with black walls and clear bottom. Plates were incubated overnight and initial (To) read was performed the following day using CyQUANT Direct Cell Proliferation Assay Kit (Thermo Fisher Scientific) according to manufacturer's protocol. All compounds were solubilized in DMSO and prepared at 3-fold serial dilution, using 9 points starting at 30 μM as highest concentration, administered to cells and incubated for 120 to 168 hours, at which point CyQUANT assay measurement was performed using Acumen Cellista instrument (Ex/Em 480/520 nm). Media-only wells were used as blank, and 0.3% DMSO was used as DMSO-treated cell control. Growth inhibition (percent response) was calculated using the following formula in Excel: “=if ([Compound treated Day N]>[Untreated Day 0, AVG], ([Compound treated Day N]−[Untreated Day 0, AVG])/([Untreated Day N]−[Untreated Day 0, AVG])*100, ([Compound Treated Day N]−[Untreated Day 0, AVG])/([Untreated Day 0, AVG])*100)”. GI₅₀ is the response corresponding to the 50% of untreated control.

GI₅₀ data are reported in Tables 4a, 4b, 4c, 4d, and 4e for compounds in Table 1. In Tables 4a, 4b, 4c, 4d, and 4e below, According to the code, A represents a GI₅₀ value of ≤0.1 μM, B represents a GI₅₀ value>0.1 μM and ≤1 μM, C represents an GI₅₀ value>1 μM.

TABLE 4a
SKCO1 GI₅₀ data

| | SKCO1 |
|---|---|
| Cpmd No. | GI₅₀ (μM) |
| 101 | A |
| 102 | A |
| 103 | A |
| 106 | B |
| 124 | A |
| 135 | A |
| 137 | B |
| 142 | B |
| 148 | B |
| 164 | A |
| 178 | B |
| 185 | B |
| 186 | A |
| 189 | A |
| 190 | B |
| 198 | A |
| 201 | A |
| 205 | A |
| 210 | A |
| 214 | A |
| 256 | A |
| 283 | A |
| 284 | A |
| 287 | A |
| 288 | A |
| 348 | A |
| 341 | A |
| 332 | B |
| 340 | A |
| 334 | B |
| 357 | A |
| 329 | A |
| 337 | A |
| 339 | B |
| 338 | A |
| 344 | A |

TABLE 4b
CW2 GI₅₀ data

| | CW2 |
|---|---|
| Cpmd No. | GI₅₀ (μM) |
| 101 | A |
| 102 | A |
| 103 | B |
| 106 | B |
| 124 | A |
| 135 | A |
| 137 | A |
| 142 | C |
| 148 | B |
| 164 | A |
| 178 | B |
| 185 | B |
| 186 | A |
| 189 | A |
| 190 | B |
| 198 | A |
| 201 | A |
| 205 | A |
| 210 | A |
| 214 | A |
| 256 | A |
| 283 | B |
| 284 | A |
| 287 | A |
| 288 | A |
| 348 | A |
| 341 | B |
| 332 | A |
| 340 | B |
| 334 | B |
| 357 | A |
| 329 | B |
| 337 | B |
| 339 | B |
| 338 | B |
| 344 | A |

TABLE 4c
GP2D GI₅₀ data

| | GP2D |
|---|---|
| Cpmd No. | GI₅₀ (μM) |
| 101 | A |
| 102 | B |
| 103 | A |
| 106 | B |
| 124 | A |
| 135 | A |
| 137 | B |
| 142 | B |
| 148 | B |
| 164 | A |
| 178 | B |
| 185 | B |
| 186 | A |
| 189 | A |
| 190 | B |
| 198 | A |
| 201 | A |
| 205 | A |
| 210 | A |
| 214 | A |
| 256 | B |
| 283 | A |
| 284 | B |
| 287 | A |
| 288 | B |
| 348 | A |
| 341 | A |
| 332 | B |
| 340 | A |
| 334 | B |
| 357 | A |
| 329 | B |
| 337 | B |
| 339 | B |
| 338 | A |
| 344 | A |

TABLE 4d
LS180 GI₅₀ data

| | LS180 |
|---|---|
| Cpmd No. | GI₅₀ (μM) |
| 101 | A |
| 102 | B |
| 103 | B |
| 106 | C |
| 124 | B |
| 135 | B |
| 137 | B |
| 142 | B |
| 148 | B |
| 164 | B |
| 178 | B |
| 185 | B |
| 186 | A |
| 189 | A |
| 190 | B |
| 198 | B |
| 201 | B |
| 205 | B |
| 210 | B |
| 214 | B |
| 256 | B |
| 283 | B |
| 284 | B |
| 287 | B |
| 288 | B |
| 348 | A |
| 341 | B |
| 332 | B |
| 340 | B |
| 334 | B |
| 357 | B |
| 329 | B |
| 337 | B |
| 339 | C |
| 338 | A |
| 344 | B |

TABLE 4e
LS174T GI₅₀ data

| | LS174T |
|---|---|
| Cpmd No. | GI₅₀ (μM) |
| 101 | A |
| 102 | B |
| 103 | B |
| 106 | C |
| 124 | A |
| 135 | B |
| 137 | B |
| 142 | B |
| 148 | B |
| 164 | A |
| 178 | B |
| 185 | B |
| 186 | A |
| 189 | A |
| 190 | B |
| 198 | B |
| 201 | A |
| 205 | B |
| 210 | A |
| 214 | B |
| 256 | A |
| 283 | B |
| 284 | A |
| 287 | A |
| 288 | B |
| 348 | A |
| 341 | B |
| 332 | B |
| 340 | A |
| 334 | B |
| 357 | A |
| 329 | B |
| 337 | B |
| 339 | B |
| 338 | A |
| 344 | B |

### Example 122. Pharmacokinetics of Compounds 102, 288, 335, and 303 in Male Balb/c Mice

### Methods

Male BALB/c mice were administered a discrete, oral (PO) dose of Compound 288, Compound 335, Compound 102, or Compound 303 at 10 mg/kg; all mice were fasted except for mice that were dosed with Compound 288. Male BALB/c mice were fasted overnight and administered a cassette dose, PO dose of Compound 303 and other compounds at 5 mg/kg per compound.

Blood samples were collected serially from three animals/time-point following PO administration of the test compounds to the mice and placed in tubes containing K₂EDTA. The blood samples were centrifuged, and the aliquots of the resulting plasma stored frozen at −60 to −90° C.

The plasma samples were analyzed by LC-MS/MS to determine concentrations of the test compounds. The in-life portion of the study and the bioanalysis of plasma samples was conducted at Wuxi (China).

### Data Analysis

The pharmacokinetic parameters for the tested compounds were calculated by non-compartmental analysis using Phoenix WinNonlin software.

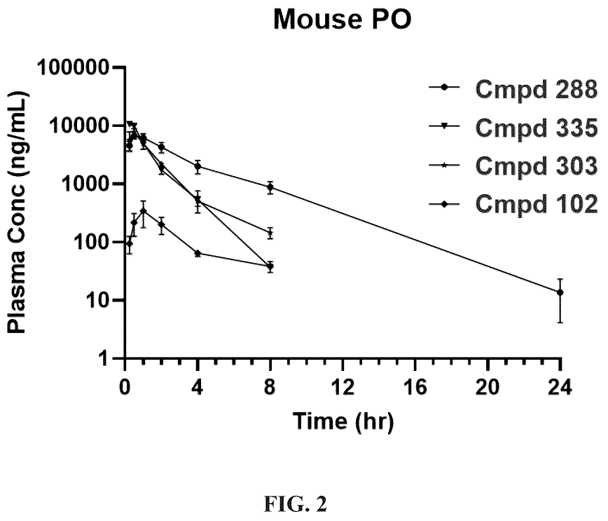

The pharmacokinetic parameters for mouse pharmacokinetic at 10 mg/kg for exemplary compounds are shown in Table 5. FIG. 2 shows the plasma concentration at different timepoints for Compound 288, Compound 335, Compound 102, and Compound 303.

TABLE 5
PK Parameters for mouse PK at 10 mg/kg for exemplary compounds

| | Mean ± SD | | | |
|---|---|---|---|---|
| PK | Compound 288 | Compound 335 | Compound 303 | Compound 102 |
| Parameters | 10 mg/kg | 10 mg/kg | 5 mg/kg | 10 mg/kg |
| T_{1/2} | 2.71 ± 0.49 | 1.08 ± 0.04 | 1.51 ± 0.22 | N/A |
| (hr) | | | | |
| Tmax | 0.83 ± 0.29 | 0.25 | 0.5 | 1.33 ± 0.58 |
| (hr) | | | | |
| Cₘₐₓ | 6867 ± 761 | 10647 ± 697 | 7790 ± 1056 | 361 ± 142 |
| (ng/mL) | | | | |
| AUCₗₐₛₜ | 29635 ± 2464 | 13349 ± 694 | 12337 ± 1711 | 889 ± 218 |
| (hr*ng/mL) | | | | |
| AUCₐₗₗ | 29692 ± 2454 | 13406 ± 696 | 12659 ± 1755 | 1039 ± 245 |
| (hr*ng/mL) | | | | |

### Example 123. Pharmacokinetics of Oral Absorption for Compound 288 in Rats

Male Sprague Dawley rats were fasted overnight and administered a discrete, PO dose of Compound 288 at 1, 10, or 30 mg/kg.

Blood samples were collected serially from three animals/time-point following PO administration of Compound 288 to the rats and placed in tubes containing K₂EDTA. The blood samples were centrifuged, and the aliquots of the resulting plasma stored frozen at −60 to −90° C.

The plasma samples were analyzed by LC-MS/MS to determine concentrations of Compound 288. The pharmacokinetic parameters for Compound 288 were calculated by non-compartmental analysis using Phoenix WinNonlin software.

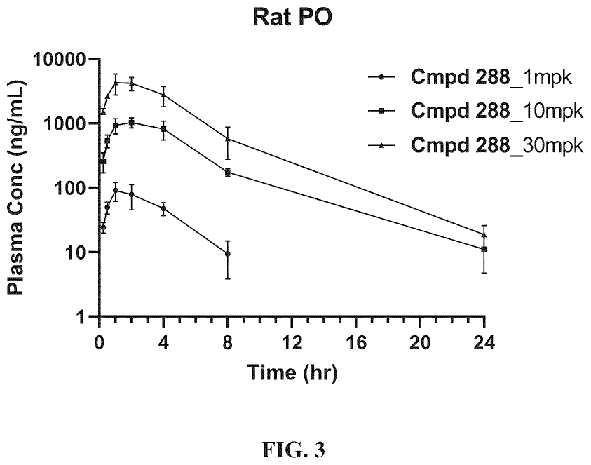

Time-dependent plasma concentrations for oral PK for Compound 288 at 1, mg/kg, 10 mg/kg, and 30 mg/kg are provided in FIG. 3.

### Example 124. Pharmacokinetics and Pharmacodynamics of Oral Absorption for Compound 288 in Cynomolgus Monkeys

Male and female cynomolgus monkey were fasted overnight and administered a discrete, PO dose of Compound 288 at 1 mg/kg.

Blood samples were collected serially from two animals/time-point following PO administration of Compound 288 to the cynomolgus monkeys and placed in tubes containing K₂EDTA. The blood samples were centrifuged, and the aliquots of the resulting plasma stored frozen at −60 to −90° C. The plasma samples were analyzed by LC-MS/MS to determine concentrations of Compound 288.

Blood samples were collected serially from two animals/time-point following PO administration of Compound 288 to the cynomolgus monkeys and placed in BD vacutainer containing K₂EDTA. The blood samples were processed to Peripheral blood mononuclear cells (PBMC). PBMC was analyzed via Western Blot for CK1α level.

The pharmacokinetic parameters for Compound 288 were calculated by non-compartmental analysis using Phoenix WinNonlin software. The pharmacodynamic parameters for Compound 288 were calculated in Excel.

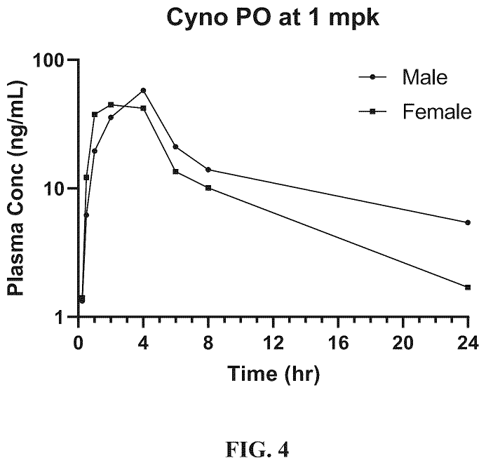

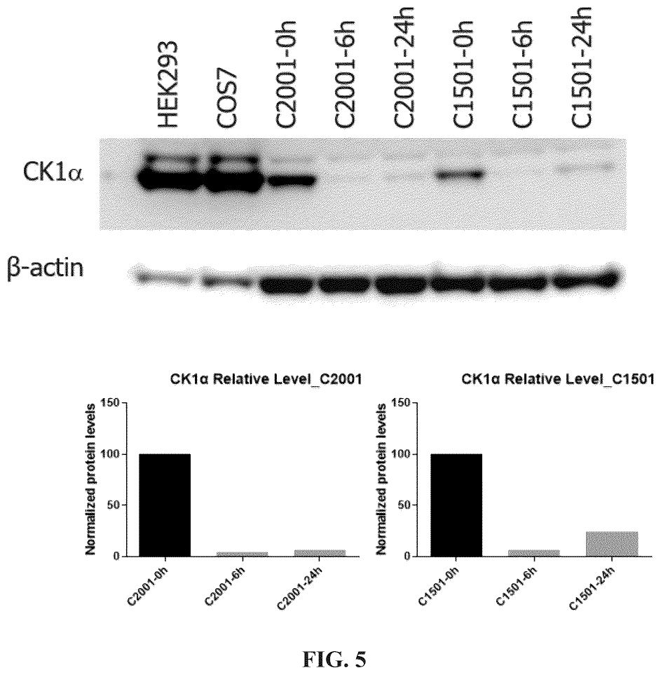

Time-dependent plasma concentrations for oral PK for Compound 288 at 1 mg/kg, for male and female cynomolgus monkeys are provided in FIG. 4. Pharmacodynamics evaluations on protein levels in CK1α and β-actin assays for cynomolgus monkeys at 1 mg/kg Compound 288 are provided in FIG. 5.

### Example 125. Multi-Dose Pharmacokinetics and Pharmacodynamics of Compounds 102 and 288

LS180 human colon cell line was purchased from ATCC and cultured in manufacturer's recommended media at 37° C. in an atmosphere of 5% CO₂ in air. 6-8 weeks old female BALB/c nude mice were used for the study. Each animal was inoculated subcutaneously on the right flank with tumor cells to allow for tumor development. Treatment was started in tumor-bearing mice when the mean tumor volume reached about 300-400 mm³. Vehicle used was 5% DMSO/95% (30% HP-β-CD in water). 15 animals were included in each of the treatment groups and were dosed orally by gavage at 1, 10, 25, or 50 mg/kg once per day (Q.D.), two consecutive days. All study animals were monitored for tumor growth, behavior, food and water consumption, body weight (BW), eye/hair matting and any other abnormal effects. Plasma and tumor samples were collected at Oh (pre-dose), 6 h, and 24 h post dose 1, and 6 h, 24 h, and 48 h post dose 2, analyzed for pharmacokinetic (PK, plasma and tumor samples) and pharmacodynamic (PD, tumor samples only) parameters. PK analysis of tumor and plasma samples was performed by LC-MS/MS to determine drug concentration in corresponding tissue samples. PD analysis was performed from lysed tumor tissue by Western blot using anti-CK1α (Abcam), anti-p53 (Abcam), anti-p21 (Cell Signaling Technologies), and loading control antibodies.

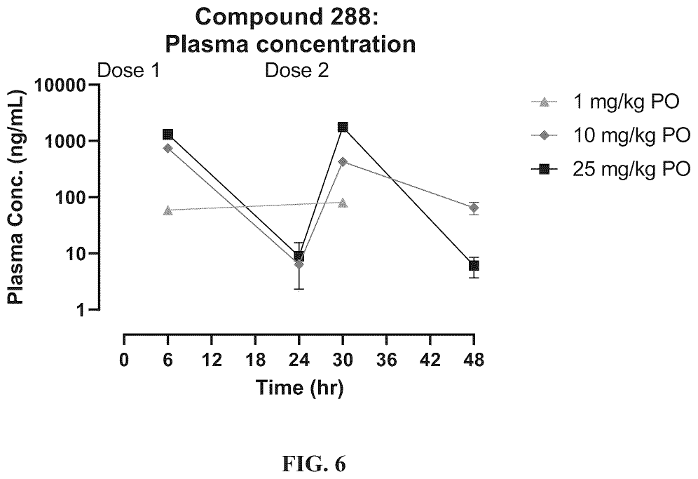

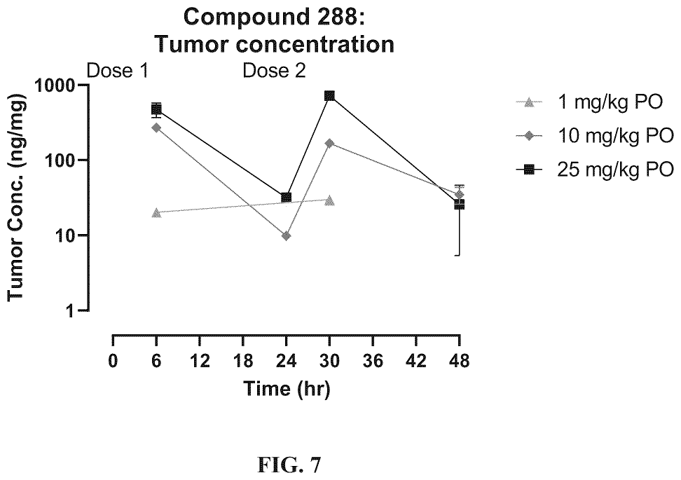

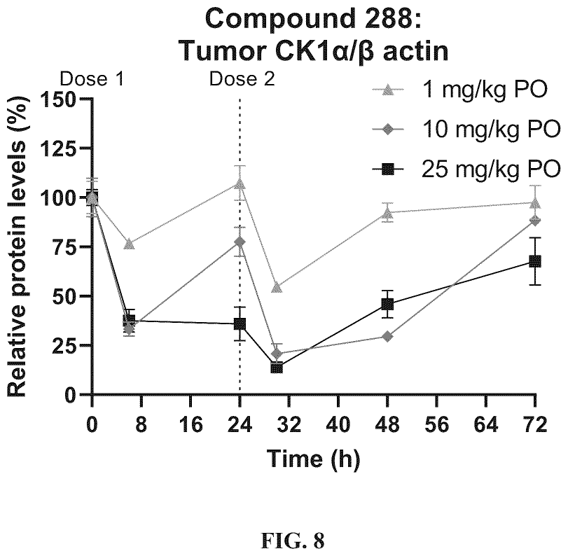

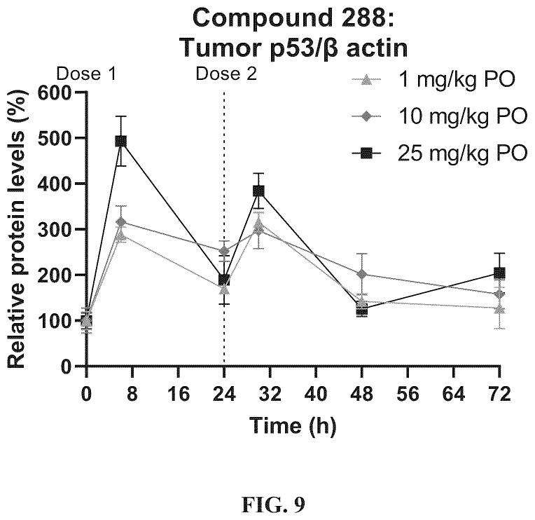

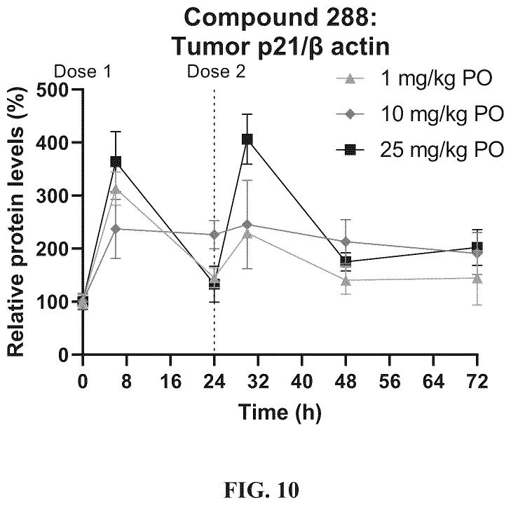

For Compound 288, multi-dose plasma concentrations with time are provided in FIG. 6 and multi-dose xenograft tumor concentrations with time are provided as FIG. 7. Multi-dose pharmacodynamic data for CK1α and β-actin levels in tumors is provided as FIG. 8. Multi-dose pharmacodynamic data for p53 and β-actin levels in tumors is provided as FIG. 9. Multi-dose pharmacodynamic data for p21 and β-actin levels in tumors is provided as FIG. 10.

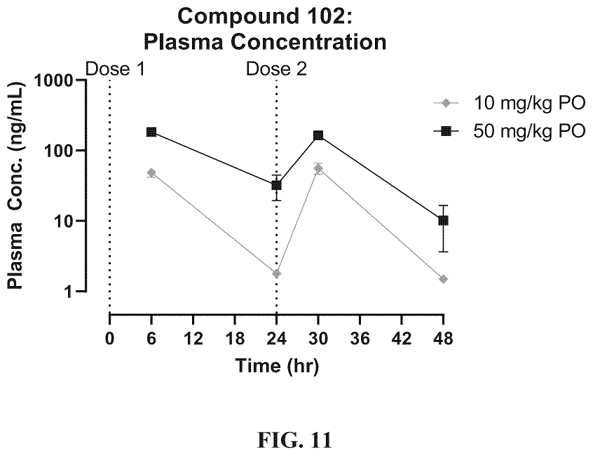

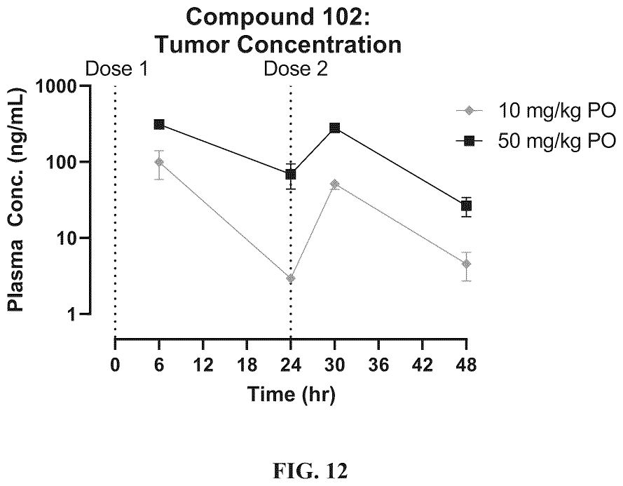

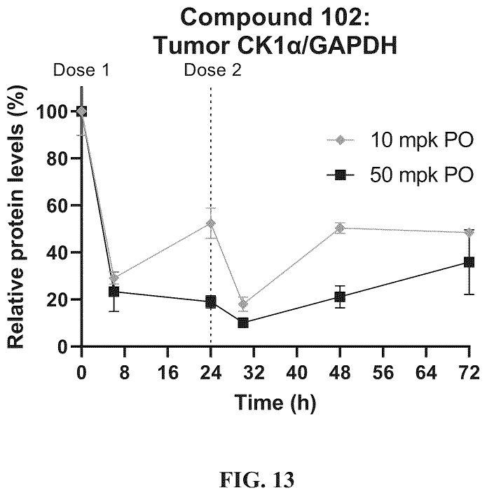

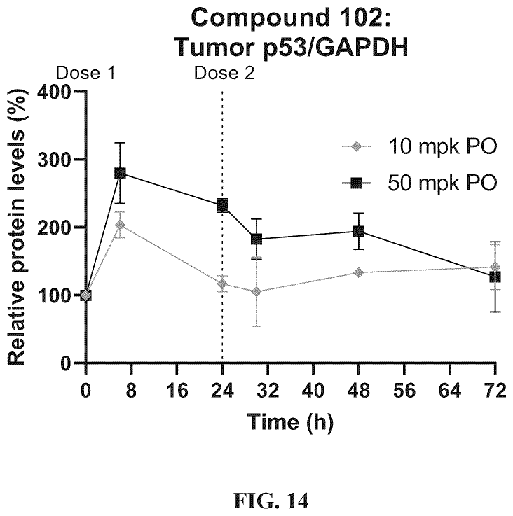

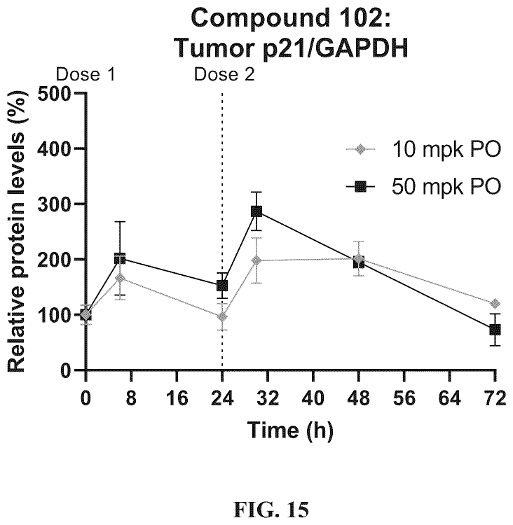

For Compound 102, multi-dose plasma concentrations with time are provided in FIG. 11 and multi-dose xenograft tumor concentrations with time are provided as FIG. 12. Multi-dose pharmacodynamic data for CK1α and GAPDH levels in tumors is provided as FIG. 13. Multi-dose pharmacodynamic data for p53 and GAPDH levels in tumors is provided as FIG. 14. Multi-dose pharmacodynamic data for p21 and GAPDH levels in tumors is provided as FIG. 15.

### Example 126. Additional Efficacy of Compound 288

CW2 human colon cell line was purchased from Riken. HT29 human colon cell line was purchased from ATCC. Cell lines were cultured in manufacturer's recommended media at 37° C. in an atmosphere of 5% CO₂ in air. 6-8 weeks old female BALB/c nude mice were used for the study. Each animal was inoculated subcutaneously on the right flank with tumor cells to allow for tumor development. Treatment was started in tumor-bearing mice when the mean tumor volume reached about 150 mm³. Vehicle used was 5% DMSO/95% (30% HP-β-CD in water). 5 animals were included in each of the treatment groups and were dosed orally by gavage at 10 mg/kg daily (Q.D.) until tumors in vehicle-treated group reached 2,500 mm³, at which point the animals were euthanized. All study animals were monitored for tumor growth, behavior, food and water consumption, body weight (BW), eye/hair matting and any other abnormal effects. Tumor size measurement was conducted twice weekly with a caliper and recorded. Tumor volume (mm³) was calculated using the formula: TV=a×b²/2, where “a” and “b” are long and short diameters of a tumor, respectively.

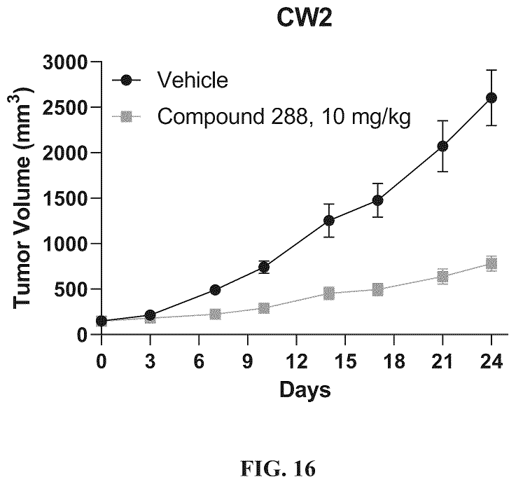

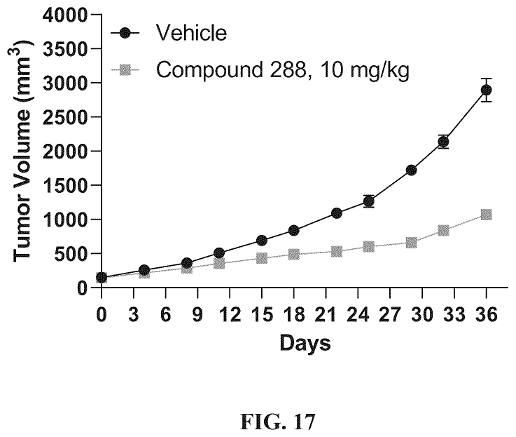

For Compound 288, tumor reduction efficacy at 10 mg/kg in the CW2 cell line is provided as FIG. 16 and tumor reduction efficacy at 10 mg/kg in the HT29 cell line is provided as FIG. 17.

### EQUIVALENTS AND SCOPE

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula I: 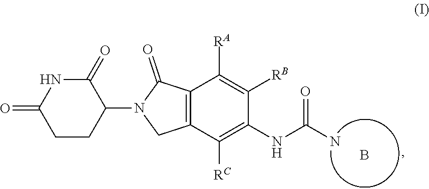
or a pharmaceutically acceptable salt thereof, wherein
each of R^{A}, R^{B}, R^{C} is independently selected from the group consisting of H and halogen; and
ring B is selected from the group consisting of 4-6 membered monocyclic heterocyclyl, 4-6 membered bridged monocyclic heterocyclyl, 8-10 membered bicyclic heterocyclyl, 6-11 membered bridged bicyclic heterocyclyl, and 7-14 membered spirocyclic heterocyclyl;
wherein 4-6 membered monocyclic heterocyclyl or 4-6 membered bridged monocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, bicyclo[1.1.1]pentanyl, phenyl, pyrazolyl, pyridinyl, pyrimidyl and thiazolyl, wherein the C₁₋₆alkyl, C₁₋₆alkoxy, bicyclo[1.1.1]pentanyl, phenyl, pyrazolyl, pyridinyl, and pyrimidyl may optionally be substituted by one, two or three substituents each independently selected from the group consisting of halogen, hydroxyl, cyano, C₁₋₆alkyl, and C₁₋₆haloalkyl;
each of 8-10 membered bicyclic heterocyclyl, 6-11 membered bridged bicyclic heterocyclyl, and 7-14 membered spirocyclic heterocyclyl is optionally substituted by one or more substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, piperazinyl, —C(O)N(R^{a}R^{b}), oxetanyl, tetrahydrofuranyl, and tetrahydropyranyl, wherein the C₁₋₆alkyl, C₁₋₆haloalkyl, C₃₋₆cycloalkyl, C₁₋₆alkoxy, phenyl, pyrazolyl, piperazinyl, oxetanyl, tetrahydrofuranyl, and tetrahydropyranyl, is optionally substituted by one, two or three substituents each independently selected from the group consisting of halogen, hydroxyl, cyano, C₁₋₆alkyl, C₁₋₆alkoxy, C₁₋₆haloalkyl, C₁₋₆haloalkoxy, N(R^{a}R^{b}), and 5-6 membered heterocyclyl; and
each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure;
provided that the compound is not:

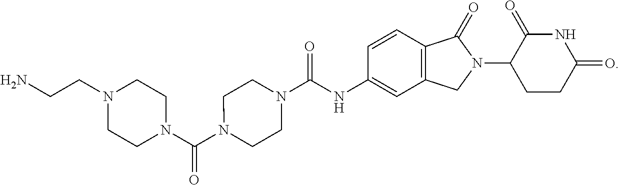 [SMILES: NCCN1CCN(C(=O)N2CCN(C(=O)Nc3ccc4c(c3)CN(C3CCC(=O)NC3=O)C4=O)CC2)CC1]

2. The compound of claim 1, wherein each of R^{A}, R^{B}, R^{C} is H.

3. The compound of claim 1, wherein each R^{A} and R^{B} is H, and R^{C} is F.

4. The compound of any one of claims 1-3, wherein ring B is 4-6 membered monocyclic heterocyclyl or 4-6 membered bridged monocyclic heterocyclyl.

5. The compound of claim 4, wherein ring B is 4-6 membered monocyclic heterocyclyl or 4-6 membered bridged monocyclic heterocyclyl optionally substituted by one, two or three substituents each independently selected from the group consisting of hydroxyl, C₁₋₆alkyl, C₁₋₆ alkyl-OH, C₁₋₆haloalkyl, C₃₋₆cycloalkyl, bicyclo[1.1.1]pentanyl, phenyl, pyrazolyl, pyridinyl, pyrimidyl and thiazolyl;
wherein each occurrence of phenyl, pyrazolyl, pyridinyl, pyrimidyl and thiazolyl is optionally substituted by one, two or three substituents each independently selected from the group consisting of halogen, hydroxyl, cyano, C₁₋₆alkyl, and C₁₋₆haloalkyl.

6. The compound of claim 4, wherein ring B is selected from the group consisting of azetidinyl, pyrrolidinyl, piperidinyl piperazinyl, and azabicyclo[2.2.1]heptane,
wherein ring B is optionally substituted by one, two or three substituents each independently selected from the group consisting of hydroxyl, C₁₋₆alkyl, C₁₋₆alkyl-OH, C₁₋₆haloalkyl, C₃₋₆cycloalkyl, phenyl, pyrazolyl, pyridinyl, pyrimidyl and thiazolyl;
wherein each occurrence of C₁₋₆alkyl, C₁₋₆alkyl-OH, C₁₋₆haloalkyl, C₃₋₆cycloalkyl, phenyl, pyrazolyl, pyridinyl, and pyrimidyl is optionally substituted by one, two or three substituents each independently selected from the group consisting of halogen, hydroxyl, cyano, C₁₋₆alkyl, and C₁₋₆haloalkyl.

7. The compound of claim 4, wherein ring B is selected from the group consisting of: 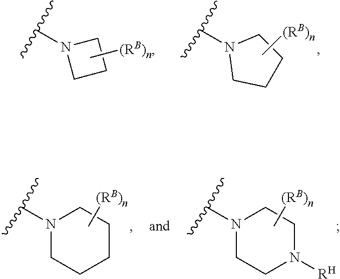
wherein: R^{B}, for each occurrence, is independently selected from the group consisting of hydroxyl, C₁₋₆alkyl, C₁₋₆alkyl-OH, C₁₋₆haloalkyl, C₃₋₆cycloalkyl, phenyl, pyrazolyl, pyridinyl, pyrimidyl and thiazolyl;
R^{H} is selected from the group consisting of C₃₋₆cycloalkyl, phenyl, pyrazolyl, pyridinyl, pyrimidyl and thiazolyl, wherein each occurrence of phenyl, pyrazolyl, pyridinyl, and pyrimidyl is optionally substituted by one, two or three substituents each independently selected from the group consisting of halogen, hydroxyl, cyano, C₁₋₆alkyl, and C₁₋₆haloalkyl; and
n is 1, 2 or 3.

8. The compound of claim 4, wherein ring B is selected from the group consisting of:

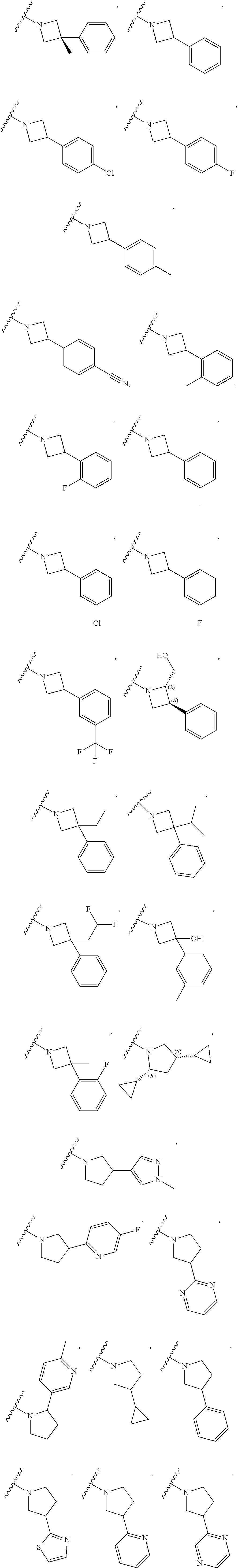

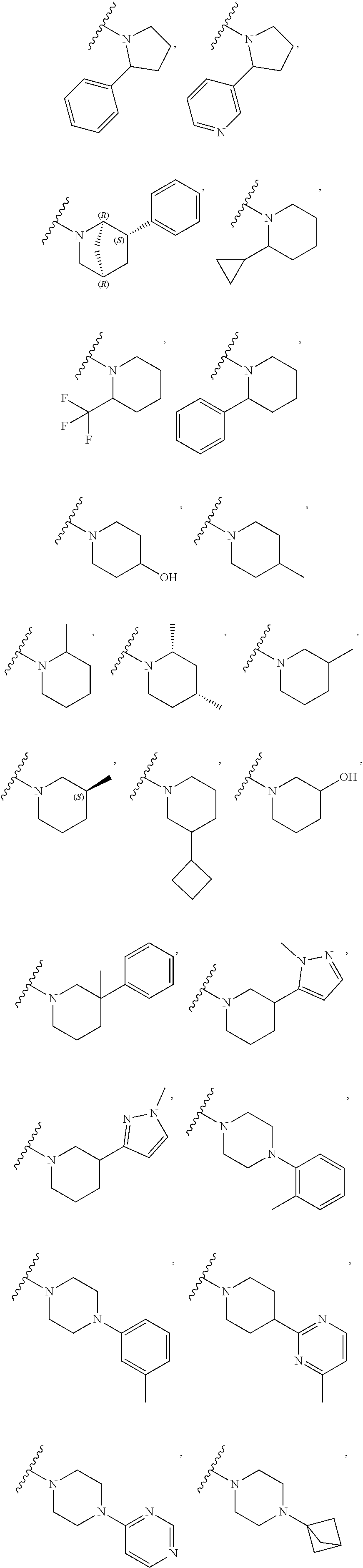

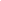

9. The compound of any one of claims 1-3, wherein ring B is 8-10 membered bicyclic heterocyclyl.

10. The compound of claim 9, wherein ring B is 8-10 membered bicyclic heterocyclyl optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl, pyrazolyl, piperazinyl, —C(O)N(R^{a}R^{b}), oxetanyl, tetrahydrofuranyl, and tetrahydropyranyl,
wherein each occurrence of C₁₋₆alkyl, C₁₋₆alkoxy, phenyl, pyrazolyl, pyrazolyl, piperazinyl, —C(O)N(R^{a}R^{b}), oxetanyl, tetrahydrofuranyl, and tetrahydropyranyl is optionally substituted by one, two or three substituents each independently selected from the group consisting of halogen, hydroxyl, cyano, C₁₋₆alkyl, C₁₋₆haloalkyl, C₁₋₆haloalkoxy, N(R^{a}R^{b}), and 5-6 membered heterocyclyl; and
each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure.

11. The compound of claim 9, wherein ring B is selected from the group consisting of indolinyl, pyrrolopyridinyl, tetrahydroquinolinyl, and tetrahydronaphthyridine,
wherein each occurrence of indolinyl, pyrrolopyridinyl, tetrahydroquinolinyl, and tetrahydronaphthyridine is optionally substituted by one, two or three substituents each independently selected from the group consisting of halogen, hydroxyl, cyano, C₁₋₆alkyl, C₁₋₆haloalkyl, C₃₋₆cycloalkyl, C₁₋₆alkoxy, phenyl pyrazolyl, piperazinyl, —C(O)N(R^{a}R^{b}), oxetanyl, tetrahydrofuranyl, and tetrahydropyranyl,
wherein each occurrence of C₁₋₆alkyl, C₁₋₆alkoxy, phenyl, pyrazolyl, pyrazolyl, piperazinyl, —C(O)N(R^{a}R^{b}), oxetanyl, tetrahydrofuranyl, and tetrahydropyranyl is optionally substituted by one, two or three substituents each independently selected from the group consisting of halogen, hydroxyl, cyano, C₁₋₆alkyl, C₁₋₆haloalkyl, C₁₋₆haloalkoxy, N(R^{a}R^{b}), and 5-6 membered heterocyclyl; and
each of R^{a} and R^{b} is independently selected from hydrogen and C₁₋₆alkyl, or R^{a} and R^{b}, taken together with the N atom to which they are attached complete a heterocycle having from 4 to 6 atoms in the ring structure.

12. The compound of claim 9, wherein ring B is selected from the group consisting of: 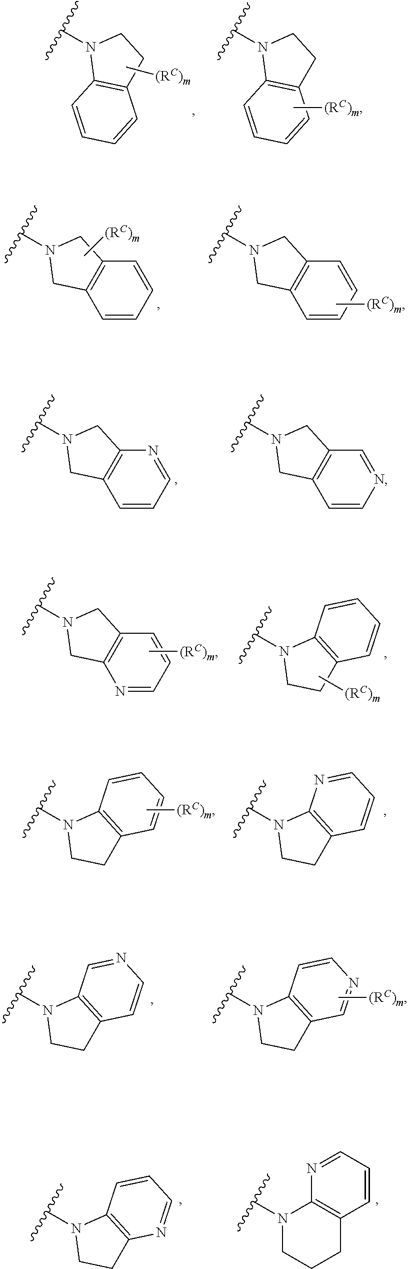 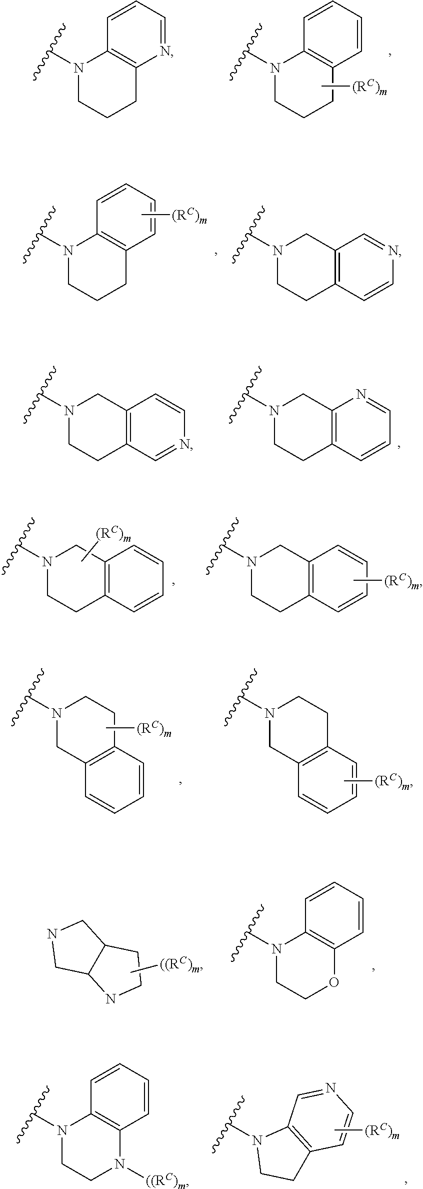 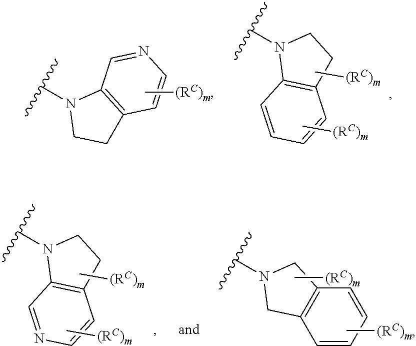
wherein R^{C}, for each occurrence, is independently selected from the group consisting of halogen, cyano, C₁₋₆alkyl, C₁₋₆alkyl-OH, C₁₋₆haloalkyl, C₁₋₆alkoxy, C₃₋₆cycloalkyl, phenyl, pyrazolyl, pyridinyl, pyrimidyl and thiazolyl;
wherein each occurrence of C₁₋₆alkoxy, phenyl, pyrazolyl, pyridinyl, and pyrimidyl is optionally substituted by one, two or three substituents each independently selected from the group consisting of halogen, hydroxyl, cyano, C₁₋₆alkyl, C₁₋₆alkoxy, and C₁₋₆haloalkyl; and
m is 0, 1,2 or 3.

13. The compound of claim 9, wherein ring B is selected from the group consisting of:

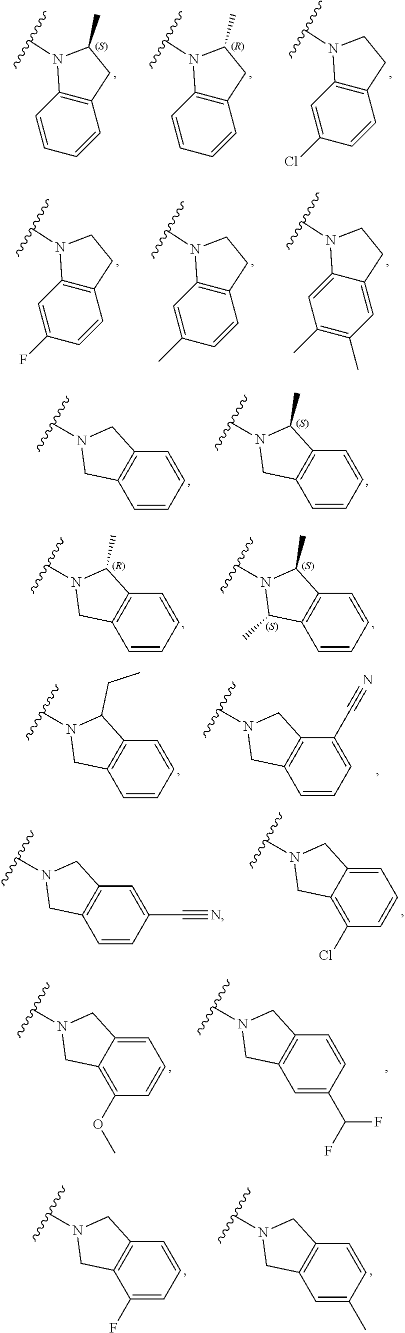

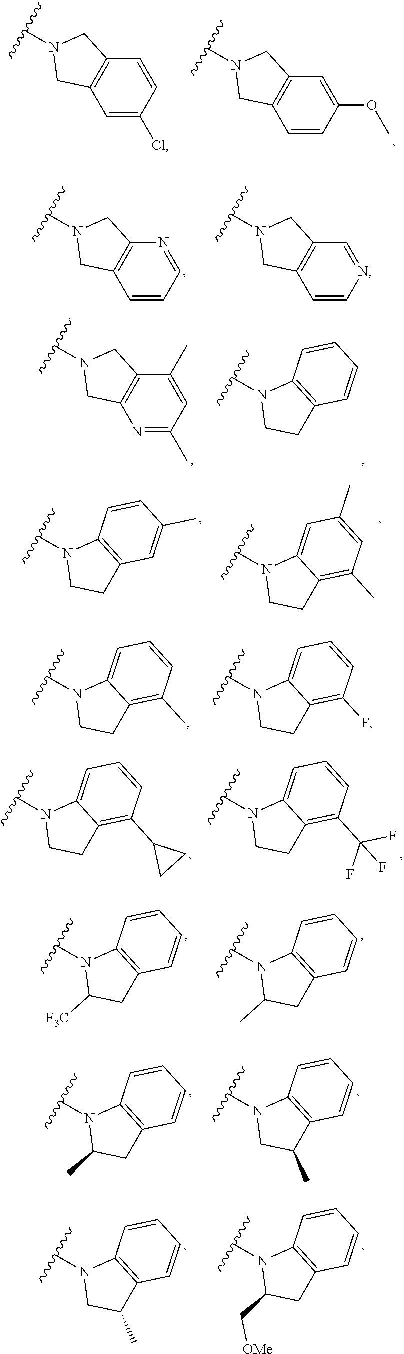

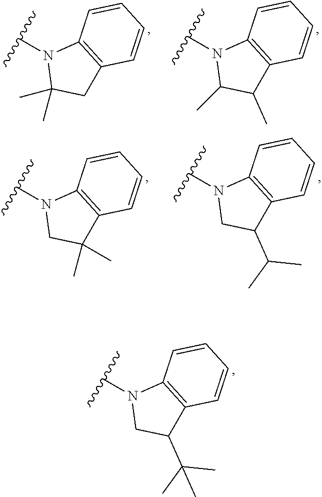

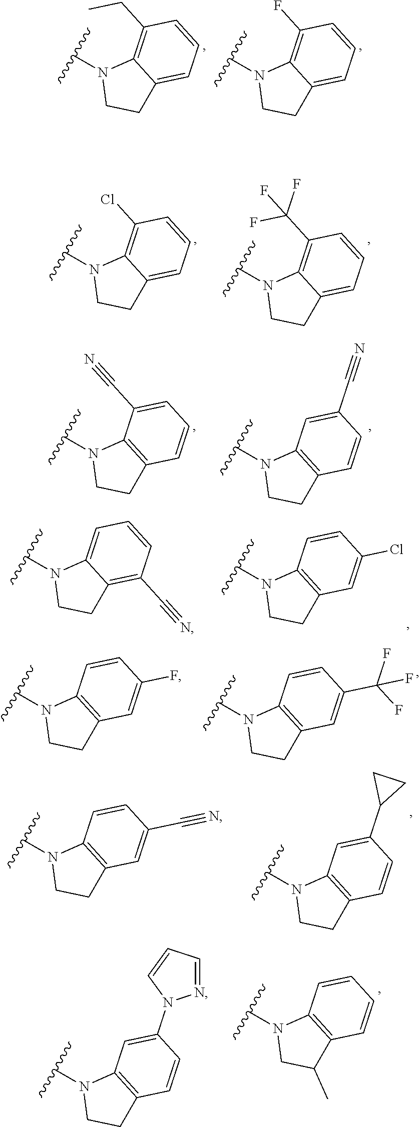

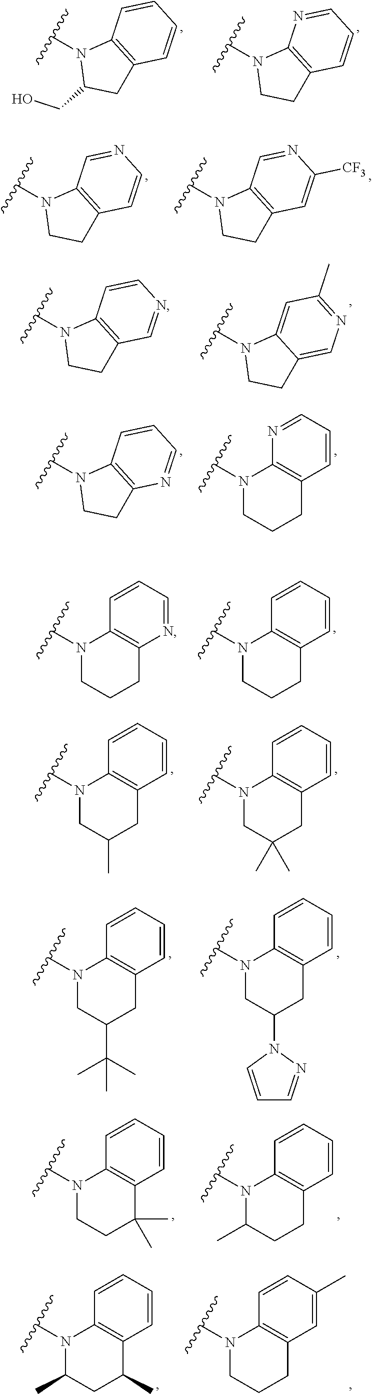

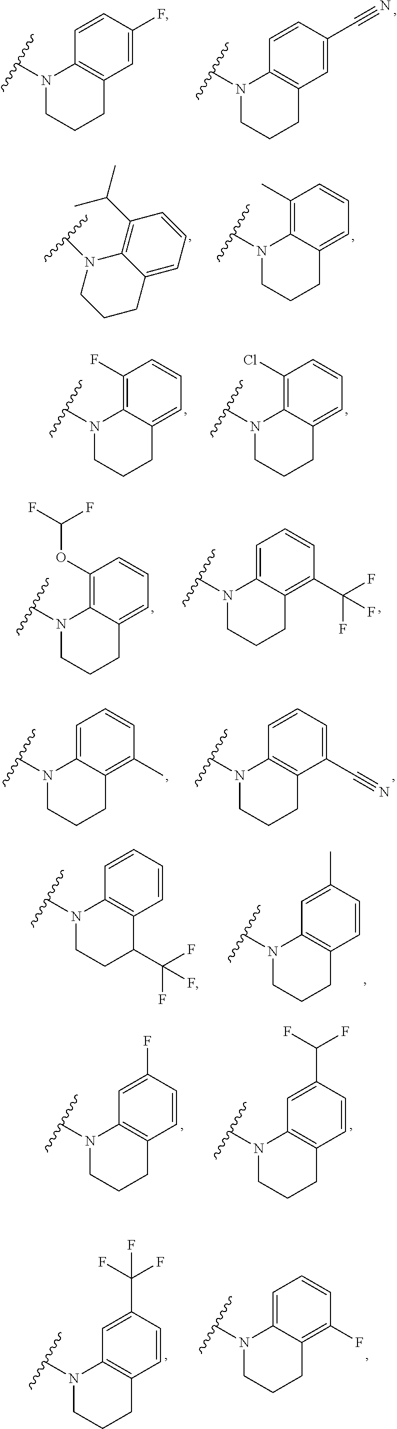

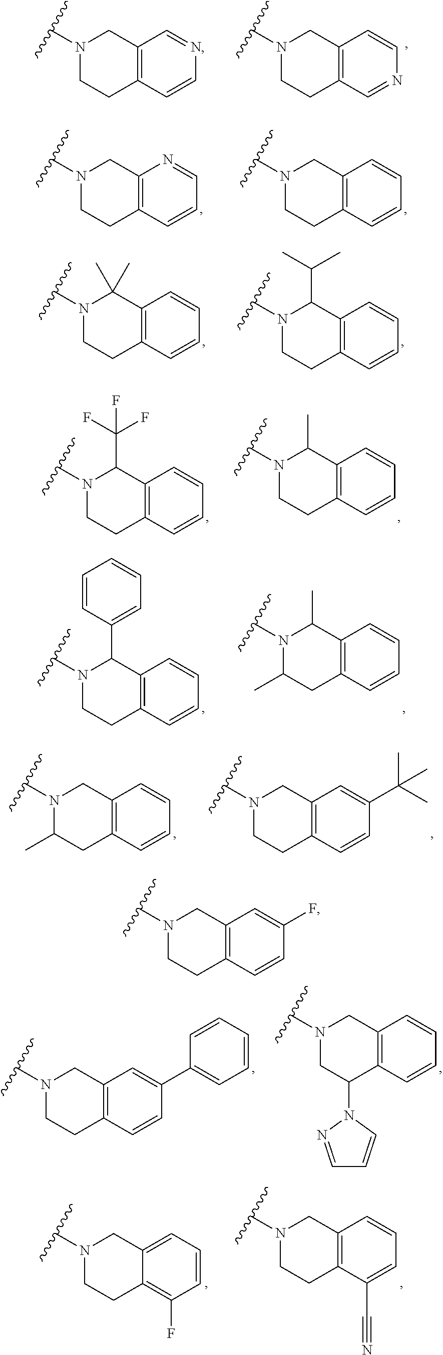

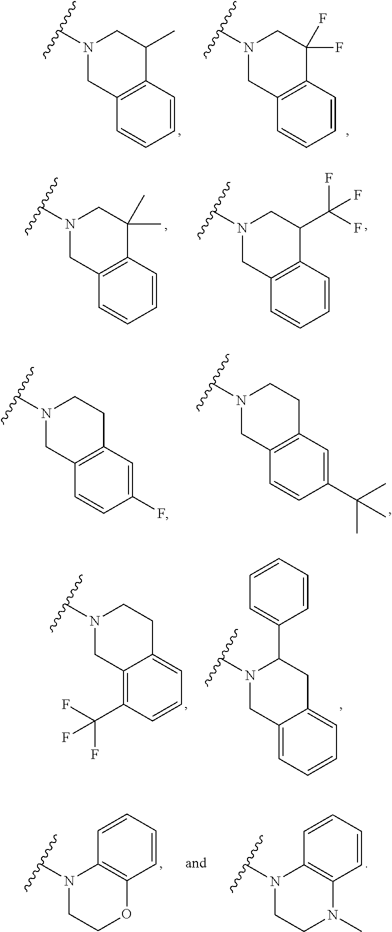

14. The compound of any one of claims 1-3, wherein ring B is 6-11 membered bridged bicyclic heterocyclyl.

15. The compound of claim 14, wherein ring B is optionally substituted by one, two or three substituents each independently selected from the group consisting of halogen, C₁₋₆alkyl, C₁₋₆haloalkyl, and phenyl.

16. The compound of claim 14, wherein ring B is selected from the group consisting of tetrahydro-epiminonaphthalene, dihydro-epiminonaphthalene, tetrahydro-cyclopropaquinolinyl, azabicycloheptanyl, octahydropyrrolopyrrolyl, azabicyclohexanyl, azabicyclohexanyl, and azabicycloheptanyl,
wherein each occurrence of tetrahydro-epiminonaphthalene, dihydro-epiminonaphthalene, tetrahydro-cyclopropaquinolinyl, azabicycloheptanyl, octahydropyrrolopyrrolyl, azabicyclohexanyl, azabicyclohexanyl, and azabicycloheptanyl is optionally substituted by one, two or three substituents each independently selected from the group consisting of halogen, C₁₋₆alkyl, C₁₋₆haloalkyl, and phenyl.

17. The compound of claim 14, wherein ring B is selected from the group consisting of: 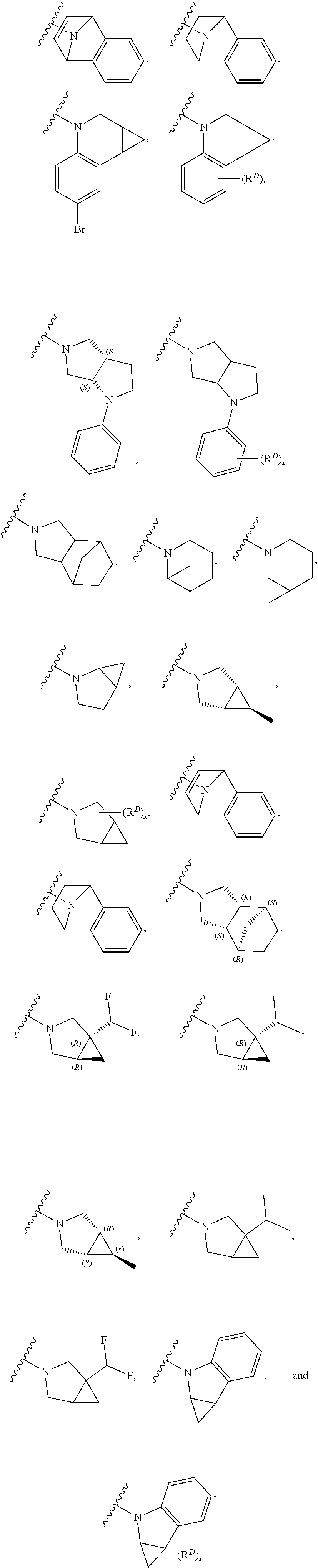
wherein R^{D}, for each occurrence, is independently selected from the group consisting of halogen, C₁₋₆alkyl, C₁₋₆haloalkyl, and phenyl; and
x is 0, 1, or 2.

18. The compound of any one of claims 1-3, wherein ring B is 7-14 membered spirocyclic heterocyclyl.

19. The compound of claim 18, wherein ring B is substituted by one, two or three occurrences of C₁₋₆alkyl, C₁₋₆haloalkyl, or C₃₋₆cycloalkyl.

20. The compound of claim 18, wherein ring B is selected from the group consisting of spiro[cyclopropane-1,3′-indolinyl], spiro[cyclobutane-1,3′-indolinyl], spiro[cyclopentane-1,3′-indolinyl], 2′,3′,5′,6′-tetrahydrospiro[indoline-3,4′-pyranyl], 2′,3′-dihydro-1′H-spiro[cyclopropane-1,4′-isoquinolinyl], 6-azaspiro[3.5]nonanyl, 5-azaspiro[2.4]heptanyl, 6-azaspiro[3.4]octanyl, and 2-azabicyclo[4.1.0]heptanyl.

21. The compound of claim 18, wherein ring B is selected from the group consisting of:

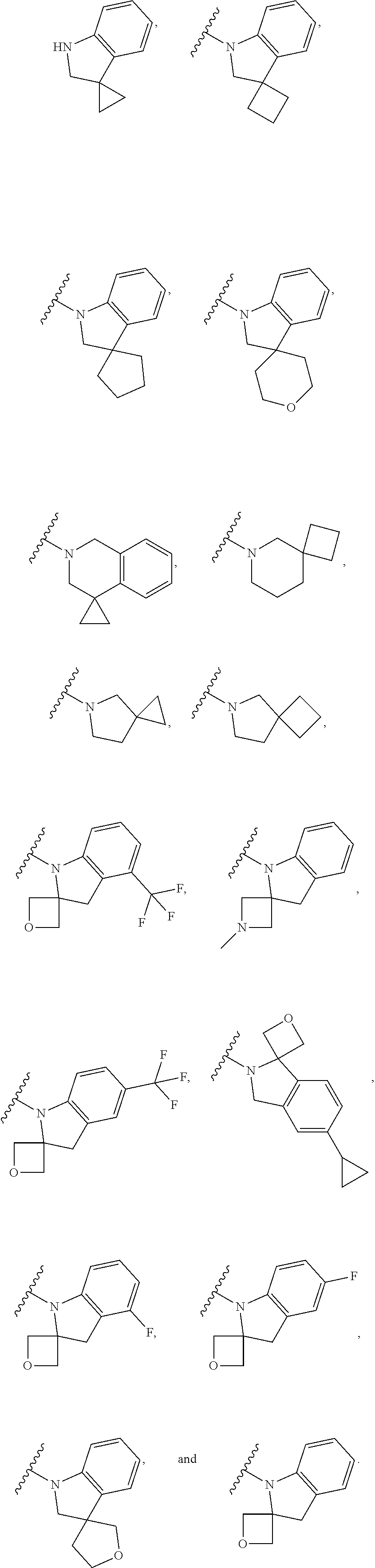

22. The compound of claim 1, wherein the compound is selected from the group consisting of: 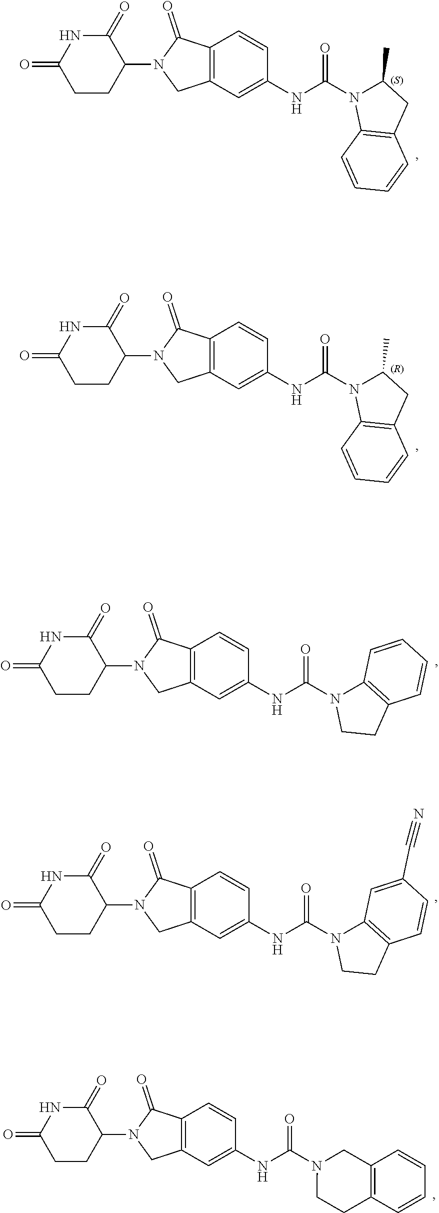 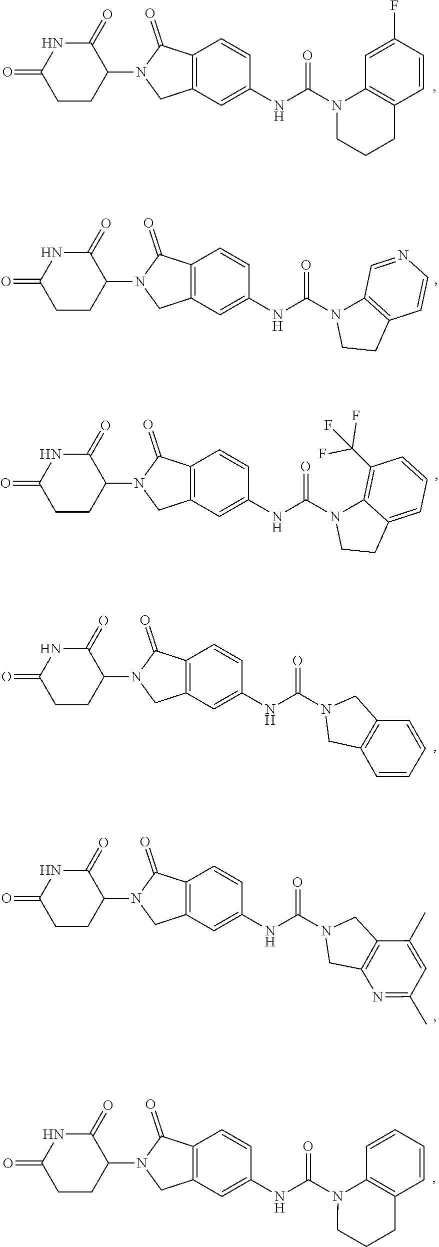 [SMILES: O=C1CCC(N2Cc3cc(NC(=O)N4CCCc5ccc(F)cc54)ccc3C2=O)C(=O)N1 ; O=C1CCC(N2Cc3cc(NC(=O)N4CCc5ccncc54)ccc3C2=O)C(=O)N1 ; O=C1CCC(N2Cc3cc(NC(=O)N4CCc5cccc(C(F)(F)F)c54)ccc3C2=O)C(=O)N1 ; O=C1CCC(N2Cc3cc(NC(=O)N4Cc5ccccc5C4)ccc3C2=O)C(=O)N1 ; Cc1cc(C)c2c(n1)CN(C(=O)Nc1ccc3c(c1)CN(C1CCC(=O)NC1=O)C3=O)C2 ; O=C1CCC(N2Cc3cc(NC(=O)N4CCCc5ccccc54)ccc3C2=O)C(=O)N1] 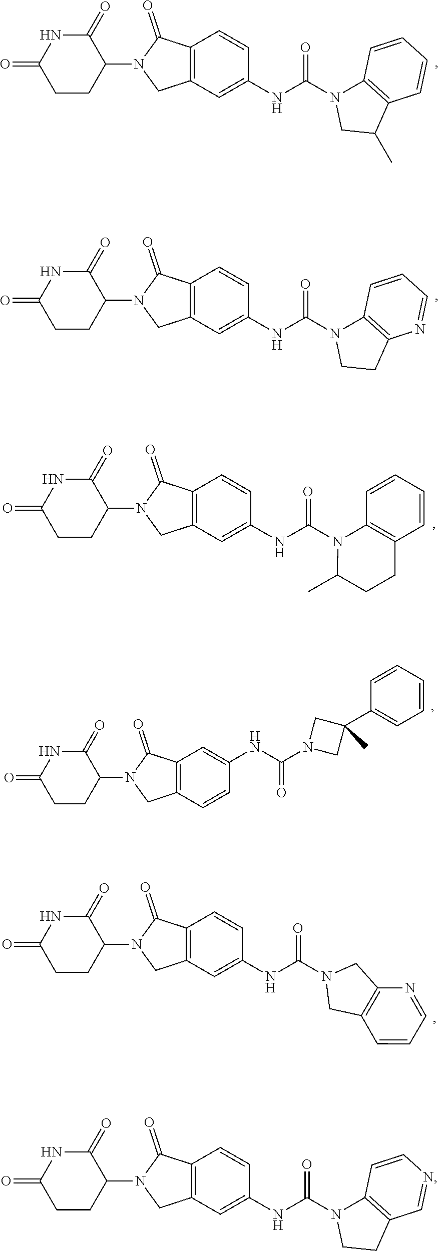 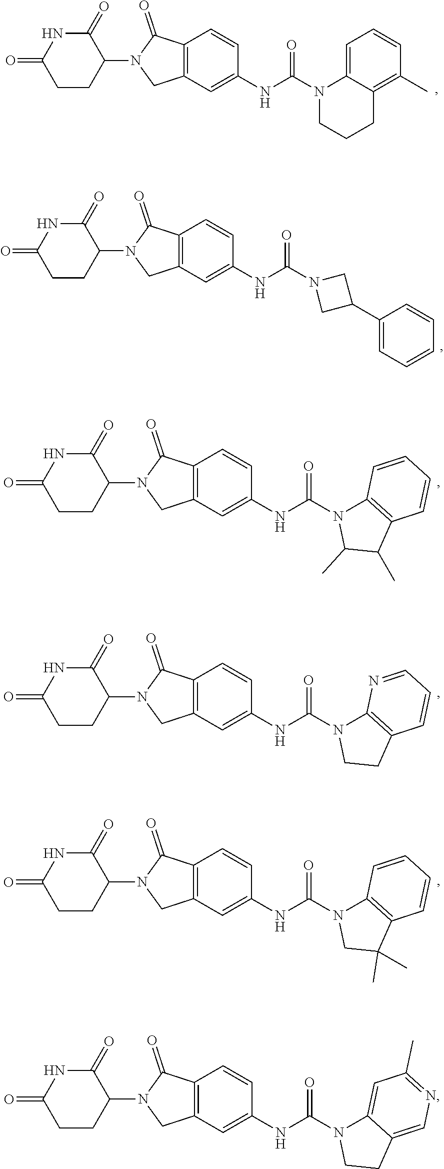 [SMILES: Cc1cccc2c1CCCN2C(=O)Nc1ccc2c(c1)CN(C1CCC(=O)NC1=O)C2=O ; O=C1CCC(N2Cc3cc(NC(=O)N4CC(c5ccccc5)C4)ccc3C2=O)C(=O)N1 ; CC1c2ccccc2N(C(=O)Nc2ccc3c(c2)CN(C2CCC(=O)NC2=O)C3=O)C1C ; O=C1CCC(N2Cc3cc(NC(=O)N4CCc5cccnc54)ccc3C2=O)C(=O)N1 ; CC1(C)CN(C(=O)Nc2ccc3c(c2)CN(C2CCC(=O)NC2=O)C3=O)c2ccccc21 ; Cc1cc2c(cn1)CCN2C(=O)Nc1ccc2c(c1)CN(C1CCC(=O)NC1=O)C2=O] 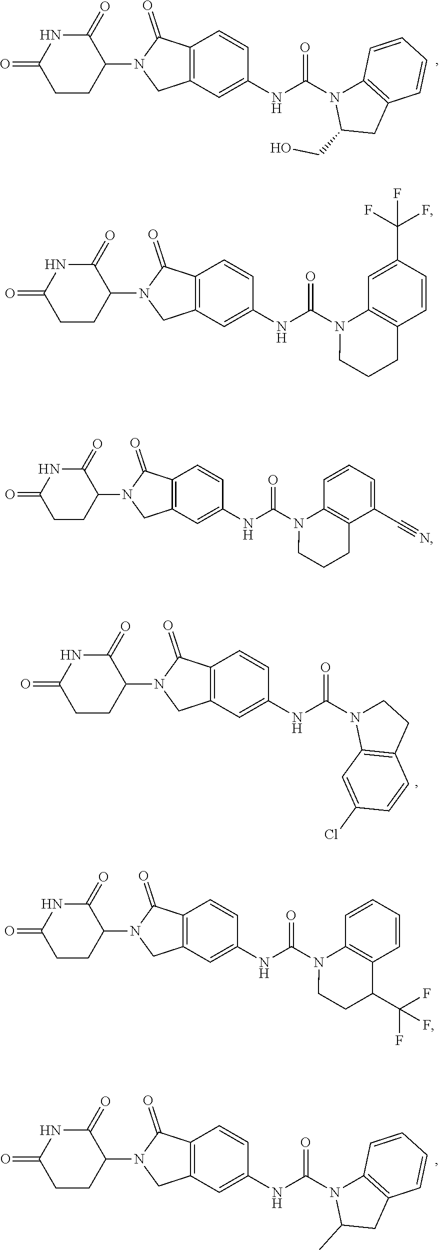 [SMILES: O=C1CCC(N2Cc3cc(NC(=O)N4c5ccccc5C[C@@H]4CO)ccc3C2=O)C(=O)N1 ; O=C1CCC(N2Cc3cc(NC(=O)N4CCCc5ccc(C(F)(F)F)cc54)ccc3C2=O)C(=O)N1 ; N#Cc1cccc2c1CCCN2C(=O)Nc1ccc2c(c1)CN(C1CCC(=O)NC1=O)C2=O ; O=C1CCC(N2Cc3cc(NC(=O)N4CCc5ccc(Cl)cc54)ccc3C2=O)C(=O)N1 ; O=C1CCC(N2Cc3cc(NC(=O)N4CCC(C(F)(F)F)c5ccccc54)ccc3C2=O)C(=O)N1 ; CC1Cc2ccccc2N1C(=O)Nc1ccc2c(c1)CN(C1CCC(=O)NC1=O)C2=O] 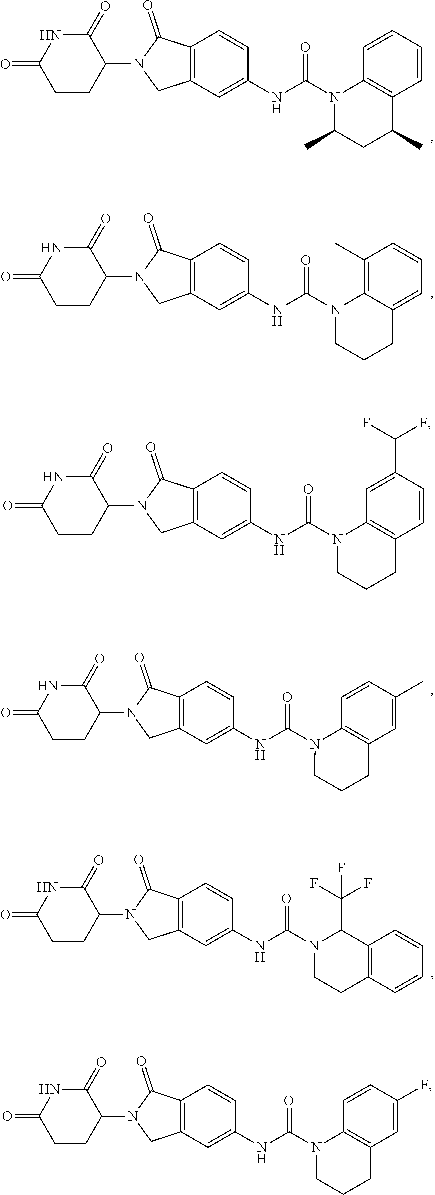 [SMILES: C[C@@H]1C[C@H](C)c2ccccc2N1C(=O)Nc1ccc2c(c1)CN(C1CCC(=O)NC1=O)C2=O ; Cc1cccc2c1N(C(=O)Nc1ccc3c(c1)CN(C1CCC(=O)NC1=O)C3=O)CCC2 ; O=C1CCC(N2Cc3cc(NC(=O)N4CCCc5ccc(C(F)F)cc54)ccc3C2=O)C(=O)N1 ; Cc1ccc2c(c1)CCCN2C(=O)Nc1ccc2c(c1)CN(C1CCC(=O)NC1=O)C2=O ; O=C1CCC(N2Cc3cc(NC(=O)N4CCc5ccccc5C4C(F)(F)F)ccc3C2=O)C(=O)N1 ; O=C1CCC(N2Cc3cc(NC(=O)N4CCCc5cc(F)ccc54)ccc3C2=O)C(=O)N1] 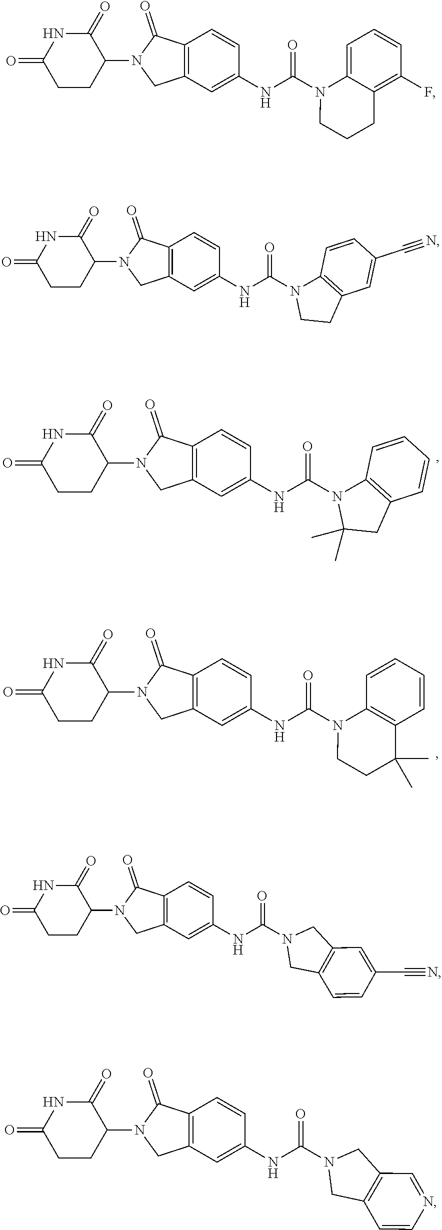 [SMILES: O=C1CCC(N2Cc3cc(NC(=O)N4CCCc5c(F)cccc54)ccc3C2=O)C(=O)N1 ; N#Cc1ccc2c(c1)CCN2C(=O)Nc1ccc2c(c1)CN(C1CCC(=O)NC1=O)C2=O ; CC1(C)Cc2ccccc2N1C(=O)Nc1ccc2c(c1)CN(C1CCC(=O)NC1=O)C2=O ; CC1(C)CCN(C(=O)Nc2ccc3c(c2)CN(C2CCC(=O)NC2=O)C3=O)c2ccccc21 ; N#Cc1ccc2c(c1)CN(C(=O)Nc1ccc3c(c1)CN(C1CCC(=O)NC1=O)C3=O)C2 ; O=C1CCC(N2Cc3cc(NC(=O)N4Cc5ccncc5C4)ccc3C2=O)C(=O)N1] 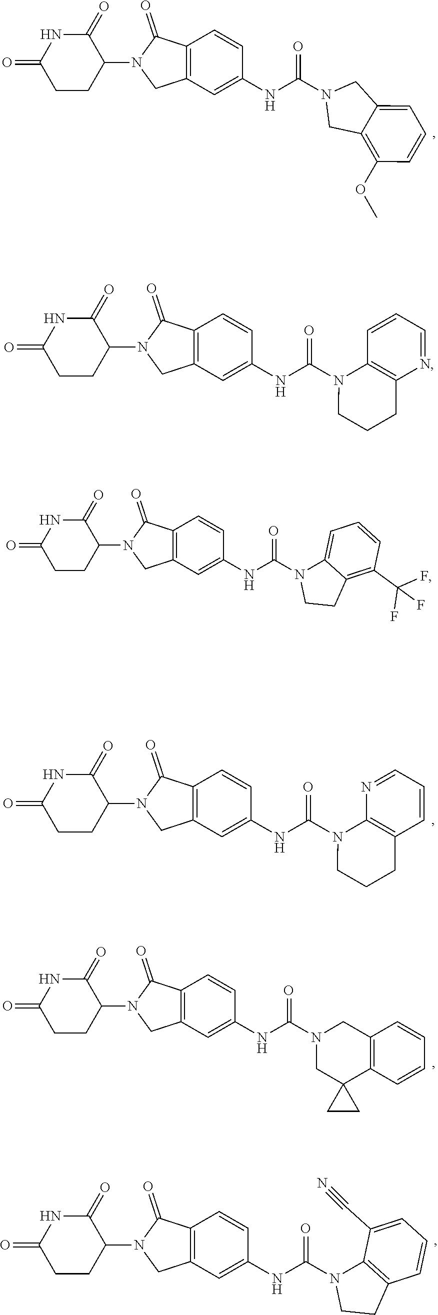 [SMILES: COc1cccc2c1CN(C(=O)Nc1ccc3c(c1)CN(C1CCC(=O)NC1=O)C3=O)C2 ; O=C1CCC(N2Cc3cc(NC(=O)N4CCCc5ncccc54)ccc3C2=O)C(=O)N1 ; O=C1CCC(N2Cc3cc(NC(=O)N4CCc5c4cccc5C(F)(F)F)ccc3C2=O)C(=O)N1 ; O=C1CCC(N2Cc3cc(NC(=O)N4CCCc5cccnc54)ccc3C2=O)C(=O)N1 ; O=C1CCC(N2Cc3cc(NC(=O)N4Cc5ccccc5C5(CC5)C4)ccc3C2=O)C(=O)N1 ; N#Cc1cccc2c1N(C(=O)Nc1ccc3c(c1)CN(C1CCC(=O)NC1=O)C3=O)CC2] 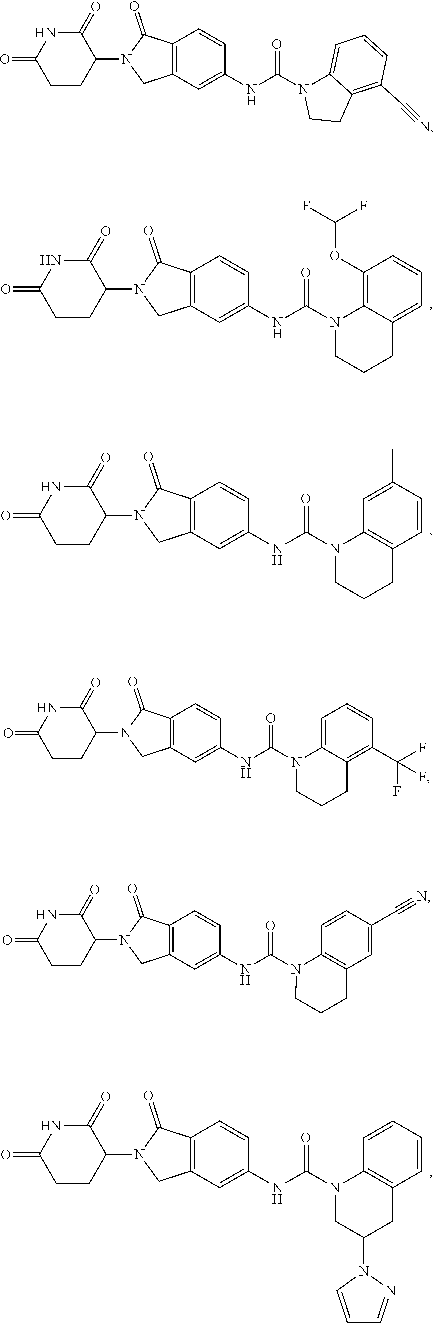 [SMILES: N#Cc1cccc2c1CCN2C(=O)Nc1ccc2c(c1)CN(C1CCC(=O)NC1=O)C2=O ; O=C1CCC(N2Cc3cc(NC(=O)N4CCCc5cccc(OC(F)F)c54)ccc3C2=O)C(=O)N1 ; Cc1ccc2c(c1)N(C(=O)Nc1ccc3c(c1)CN(C1CCC(=O)NC1=O)C3=O)CCC2 ; O=C1CCC(N2Cc3cc(NC(=O)N4CCCc5c4cccc5C(F)(F)F)ccc3C2=O)C(=O)N1 ; N#Cc1ccc2c(c1)CCCN2C(=O)Nc1ccc2c(c1)CN(C1CCC(=O)NC1=O)C2=O ; O=C1CCC(N2Cc3cc(NC(=O)N4CC(n5cccn5)Cc5ccccc54)ccc3C2=O)C(=O)N1] 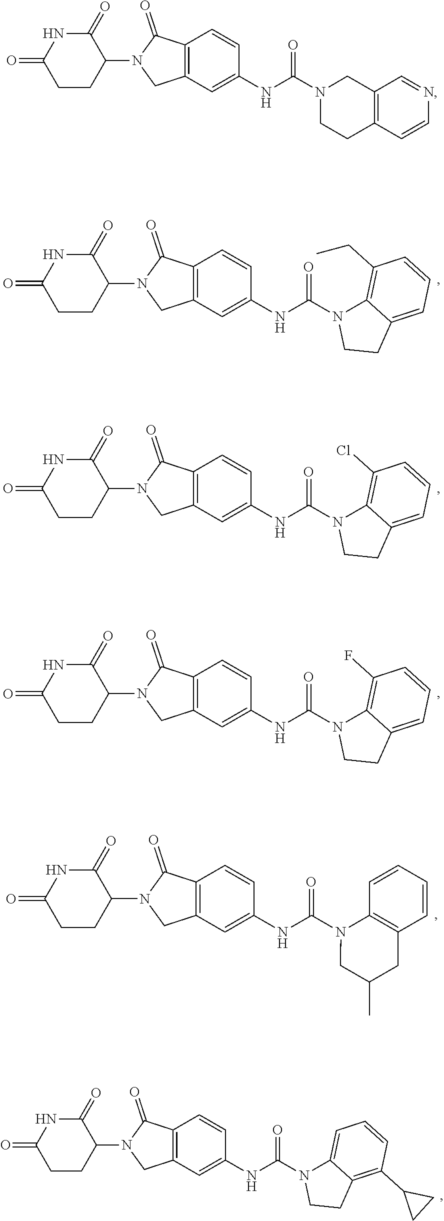 [SMILES: O=C1CCC(N2Cc3cc(NC(=O)N4CCc5ccncc5C4)ccc3C2=O)C(=O)N1 ; CCc1cccc2c1N(C(=O)Nc1ccc3c(c1)CN(C1CCC(=O)NC1=O)C3=O)CC2 ; O=C1CCC(N2Cc3cc(NC(=O)N4CCc5cccc(Cl)c54)ccc3C2=O)C(=O)N1 ; O=C1CCC(N2Cc3cc(NC(=O)N4CCc5cccc(F)c54)ccc3C2=O)C(=O)N1 ; CC1Cc2ccccc2N(C(=O)Nc2ccc3c(c2)CN(C2CCC(=O)NC2=O)C3=O)C1 ; O=C1CCC(N2Cc3cc(NC(=O)N4CCc5c(C6CC6)cccc54)ccc3C2=O)C(=O)N1] 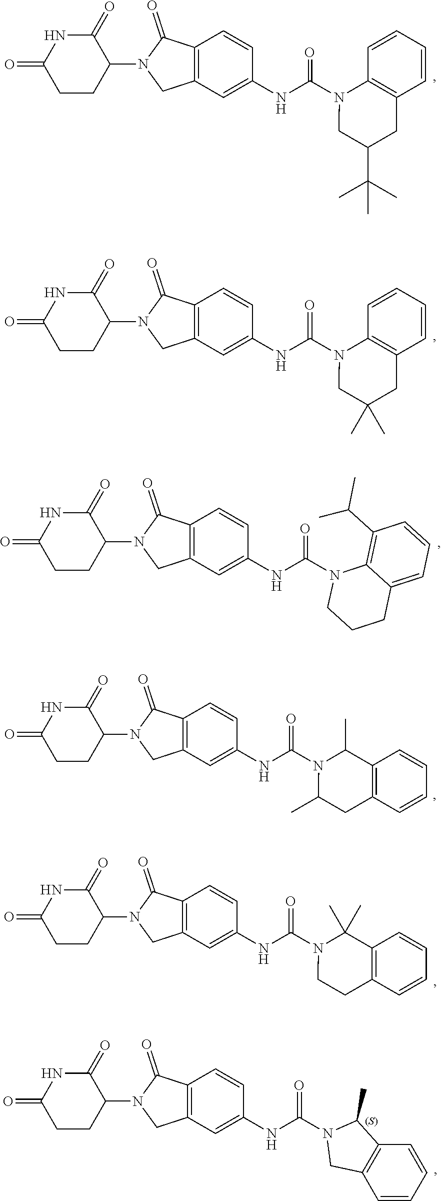 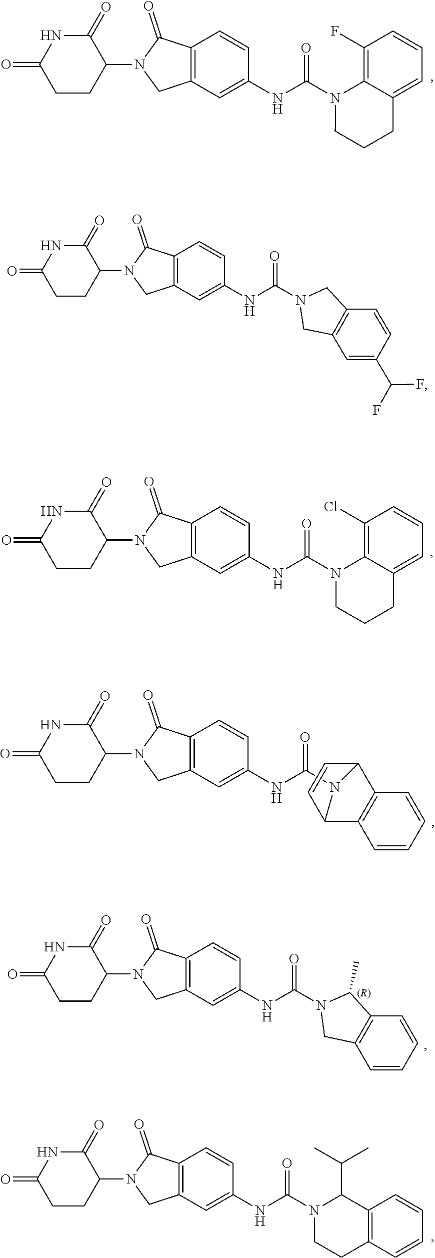 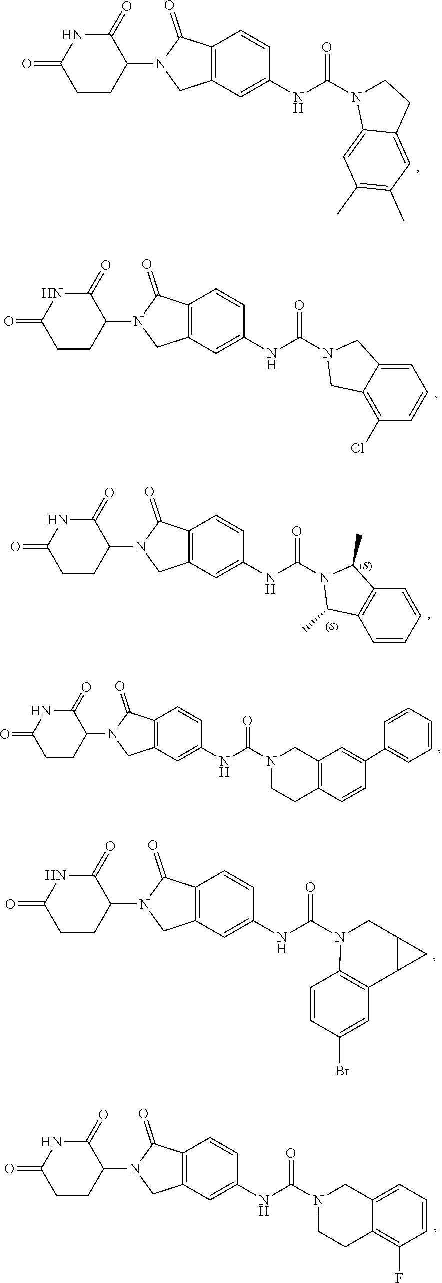 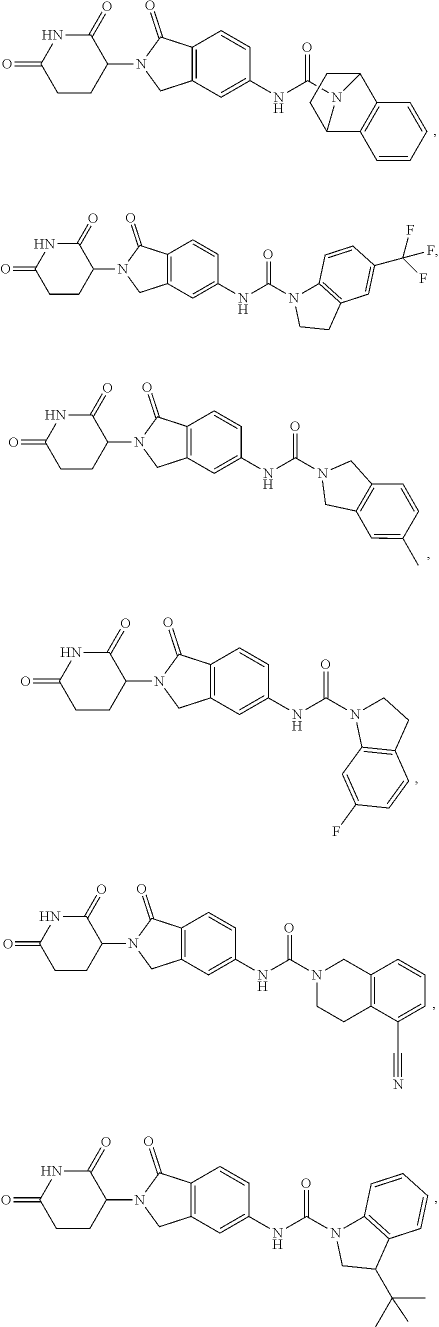 [SMILES: O=C1CCC(N2Cc3cc(NC(=O)N4C5CCC4c4ccccc45)ccc3C2=O)C(=O)N1 ; O=C1CCC(N2Cc3cc(NC(=O)N4CCc5cc(C(F)(F)F)ccc54)ccc3C2=O)C(=O)N1 ; Cc1ccc2c(c1)CN(C(=O)Nc1ccc3c(c1)CN(C1CCC(=O)NC1=O)C3=O)C2 ; O=C1CCC(N2Cc3cc(NC(=O)N4CCc5ccc(F)cc54)ccc3C2=O)C(=O)N1 ; N#Cc1cccc2c1CCN(C(=O)Nc1ccc3c(c1)CN(C1CCC(=O)NC1=O)C3=O)C2 ; CC(C)(C)C1CN(C(=O)Nc2ccc3c(c2)CN(C2CCC(=O)NC2=O)C3=O)c2ccccc21] 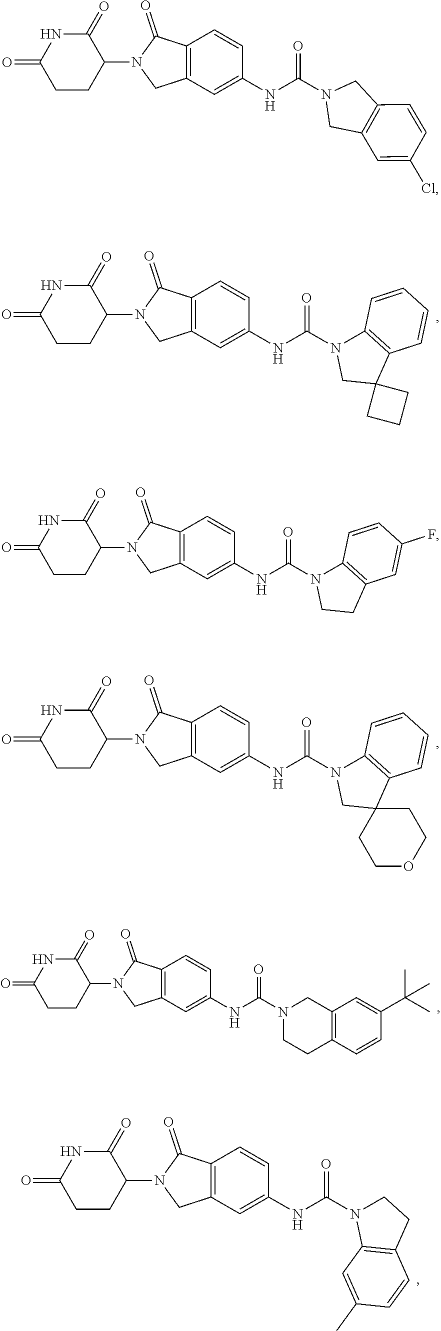 [SMILES: O=C1CCC(N2Cc3cc(NC(=O)N4Cc5ccc(Cl)cc5C4)ccc3C2=O)C(=O)N1 ; O=C1CCC(N2Cc3cc(NC(=O)N4CC5(CCC5)c5ccccc54)ccc3C2=O)C(=O)N1 ; O=C1CCC(N2Cc3cc(NC(=O)N4CCc5cc(F)ccc54)ccc3C2=O)C(=O)N1 ; O=C1CCC(N2Cc3cc(NC(=O)N4CC5(CCOCC5)c5ccccc54)ccc3C2=O)C(=O)N1 ; CC(C)(C)c1ccc2c(c1)CN(C(=O)Nc1ccc3c(c1)CN(C1CCC(=O)NC1=O)C3=O)CC2 ; Cc1ccc2c(c1)N(C(=O)Nc1ccc3c(c1)CN(C1CCC(=O)NC1=O)C3=O)CC2] 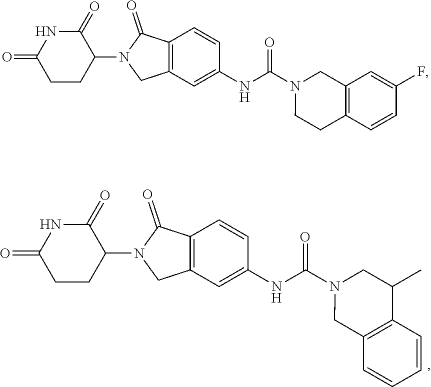 [SMILES: O=C1CCC(N2Cc3cc(NC(=O)N4CCc5ccc(F)cc5C4)ccc3C2=O)C(=O)N1 ; CC1CN(C(=O)Nc2ccc3c(c2)CN(C2CCC(=O)NC2=O)C3=O)Cc2ccccc21] 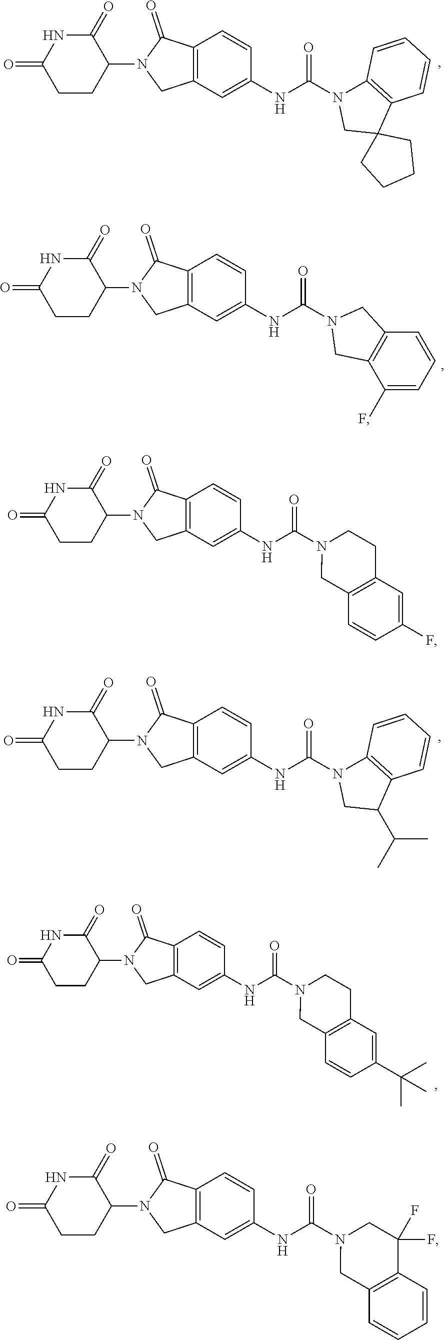 [SMILES: O=C1CCC(N2Cc3cc(NC(=O)N4CC5(CCCC5)c5ccccc54)ccc3C2=O)C(=O)N1 ; O=C1CCC(N2Cc3cc(NC(=O)N4Cc5cccc(F)c5C4)ccc3C2=O)C(=O)N1 ; O=C1CCC(N2Cc3cc(NC(=O)N4CCc5cc(F)ccc5C4)ccc3C2=O)C(=O)N1 ; CC(C)C1CN(C(=O)Nc2ccc3c(c2)CN(C2CCC(=O)NC2=O)C3=O)c2ccccc21 ; CC(C)(C)c1ccc2c(c1)CCN(C(=O)Nc1ccc3c(c1)CN(C1CCC(=O)NC1=O)C3=O)C2 ; O=C1CCC(N2Cc3cc(NC(=O)N4Cc5ccccc5C(F)(F)C4)ccc3C2=O)C(=O)N1] 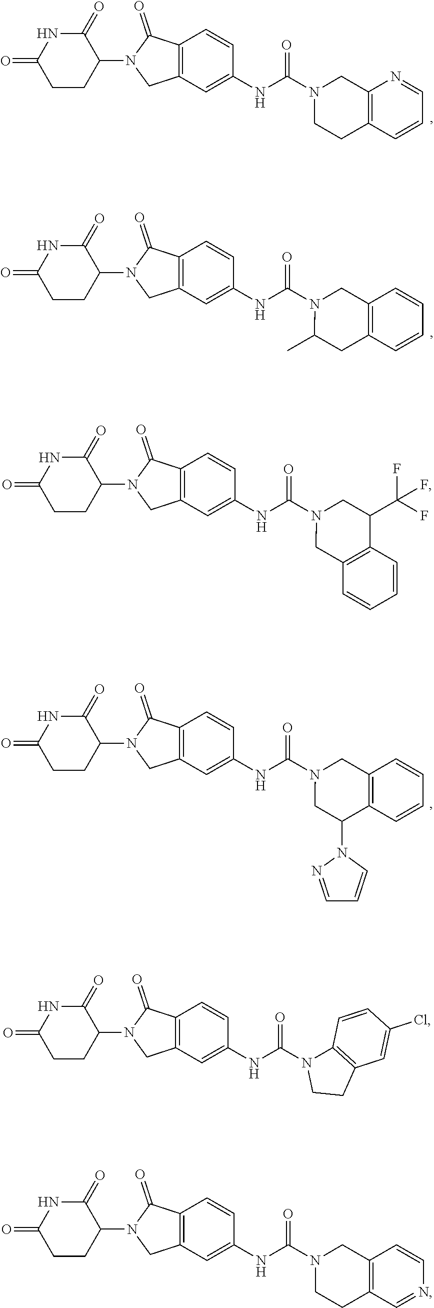 [SMILES: O=C1CCC(N2Cc3cc(NC(=O)N4CCc5cccnc5C4)ccc3C2=O)C(=O)N1 ; CC1Cc2ccccc2CN1C(=O)Nc1ccc2c(c1)CN(C1CCC(=O)NC1=O)C2=O ; O=C1CCC(N2Cc3cc(NC(=O)N4Cc5ccccc5C(C(F)(F)F)C4)ccc3C2=O)C(=O)N1 ; O=C1CCC(N2Cc3cc(NC(=O)N4Cc5ccccc5C(n5cccn5)C4)ccc3C2=O)C(=O)N1 ; O=C1CCC(N2Cc3cc(NC(=O)N4CCc5cc(Cl)ccc54)ccc3C2=O)C(=O)N1 ; O=C1CCC(N2Cc3cc(NC(=O)N4CCc5cnccc5C4)ccc3C2=O)C(=O)N1] 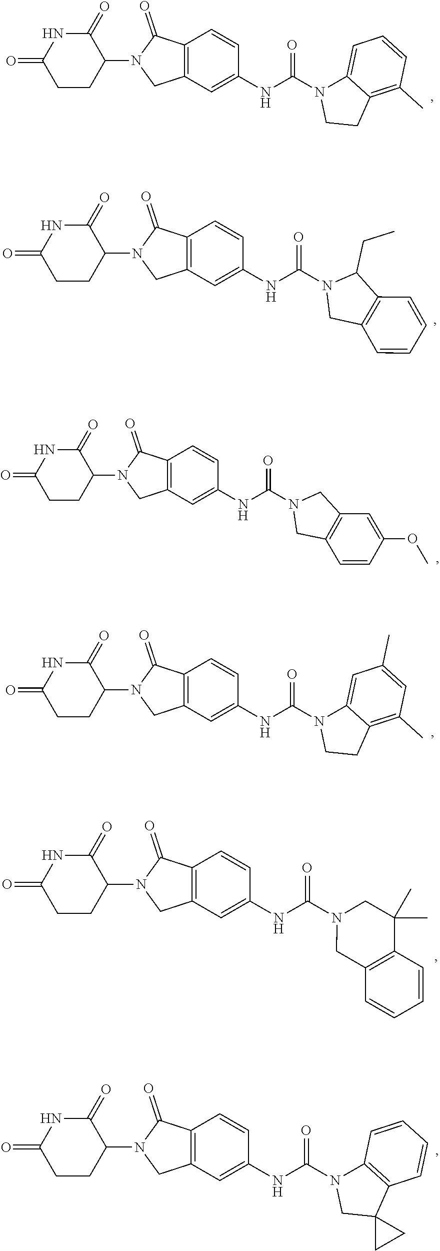 [SMILES: Cc1cccc2c1CCN2C(=O)Nc1ccc2c(c1)CN(C1CCC(=O)NC1=O)C2=O ; CCC1c2ccccc2CN1C(=O)Nc1ccc2c(c1)CN(C1CCC(=O)NC1=O)C2=O ; COc1ccc2c(c1)CN(C(=O)Nc1ccc3c(c1)CN(C1CCC(=O)NC1=O)C3=O)C2 ; Cc1cc(C)c2c(c1)N(C(=O)Nc1ccc3c(c1)CN(C1CCC(=O)NC1=O)C3=O)CC2 ; CC1(C)CN(C(=O)Nc2ccc3c(c2)CN(C2CCC(=O)NC2=O)C3=O)Cc2ccccc21 ; O=C1CCC(N2Cc3cc(NC(=O)N4CC5(CC5)c5ccccc54)ccc3C2=O)C(=O)N1] 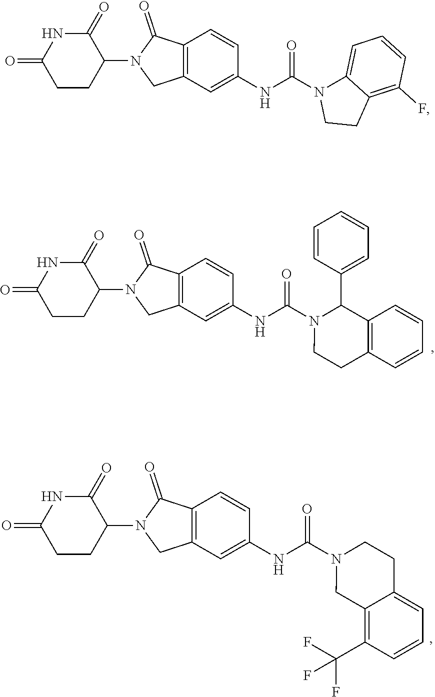 [SMILES: O=C1CCC(N2Cc3cc(NC(=O)N4CCc5c(F)cccc54)ccc3C2=O)C(=O)N1 ; O=C1CCC(N2Cc3cc(NC(=O)N4CCc5ccccc5C4c4ccccc4)ccc3C2=O)C(=O)N1 ; O=C1CCC(N2Cc3cc(NC(=O)N4CCc5cccc(C(F)(F)F)c5C4)ccc3C2=O)C(=O)N1] 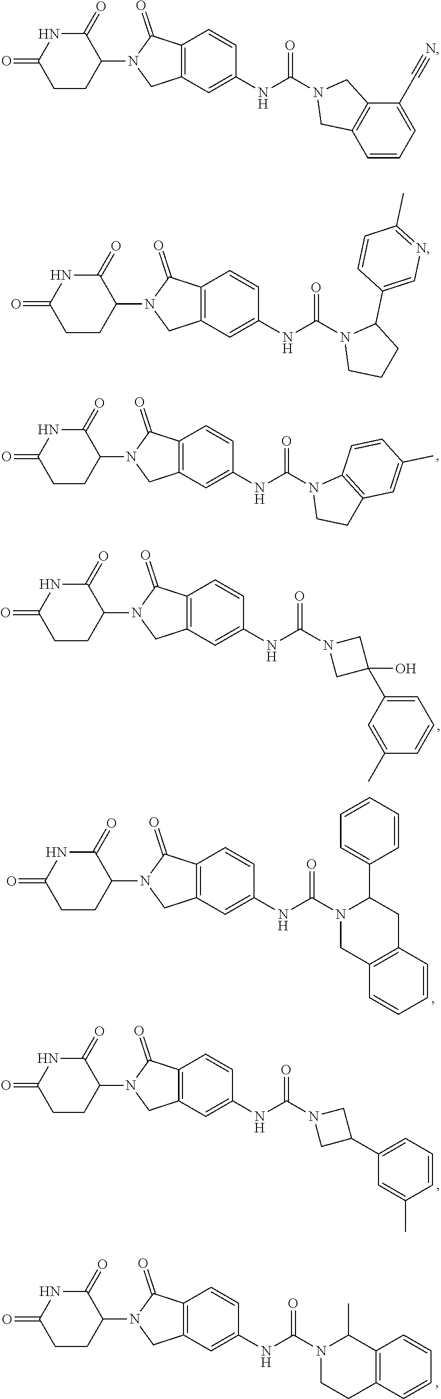 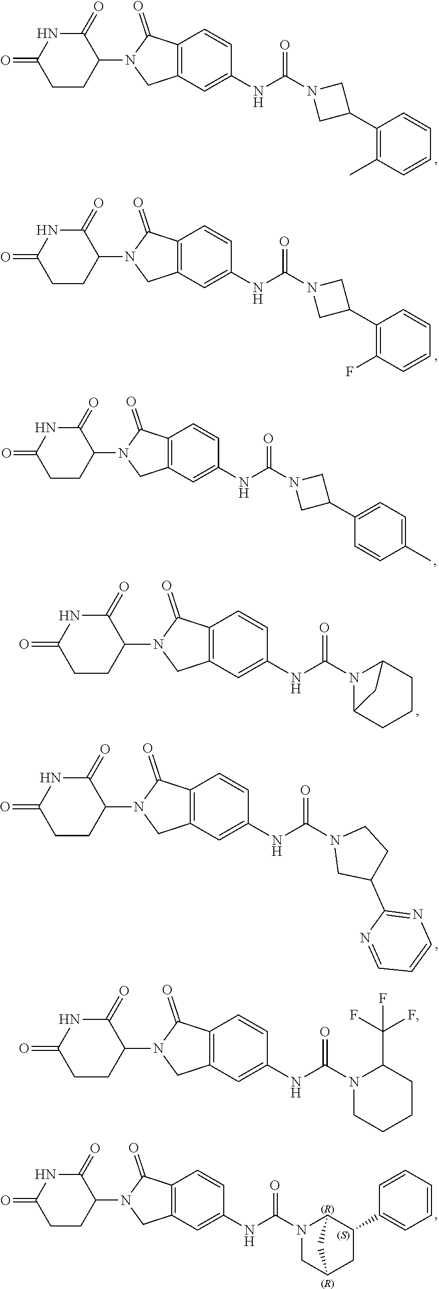 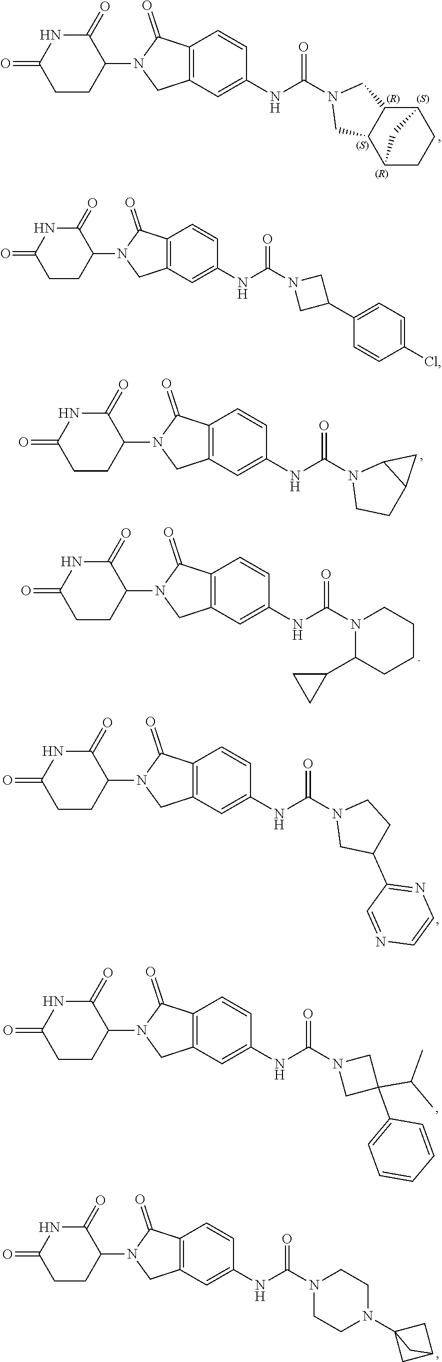 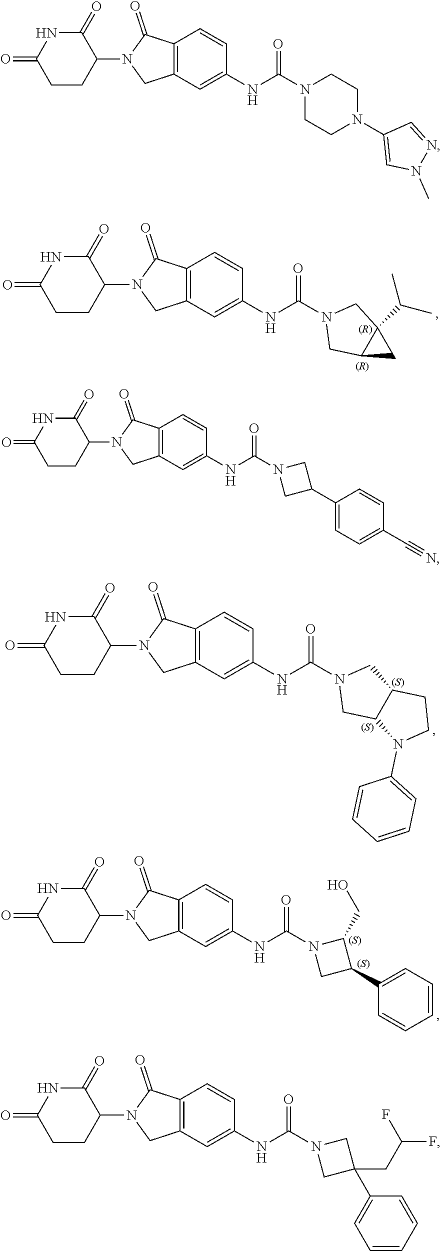 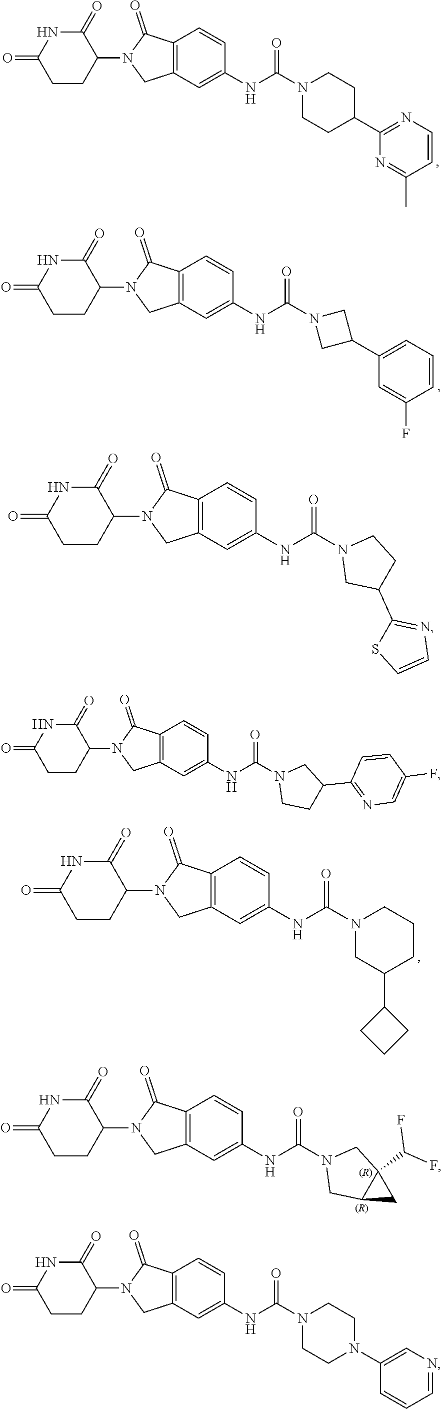 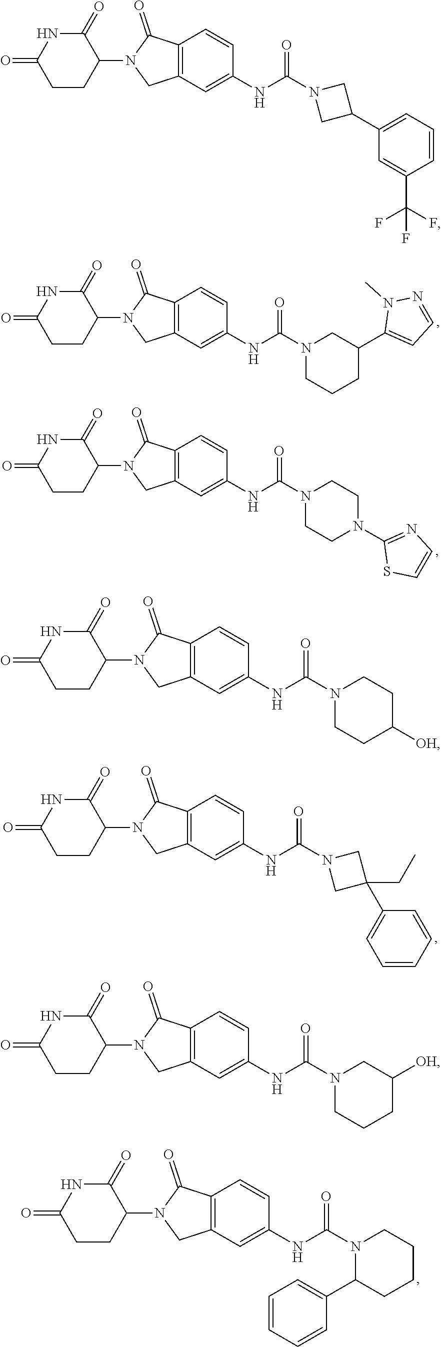 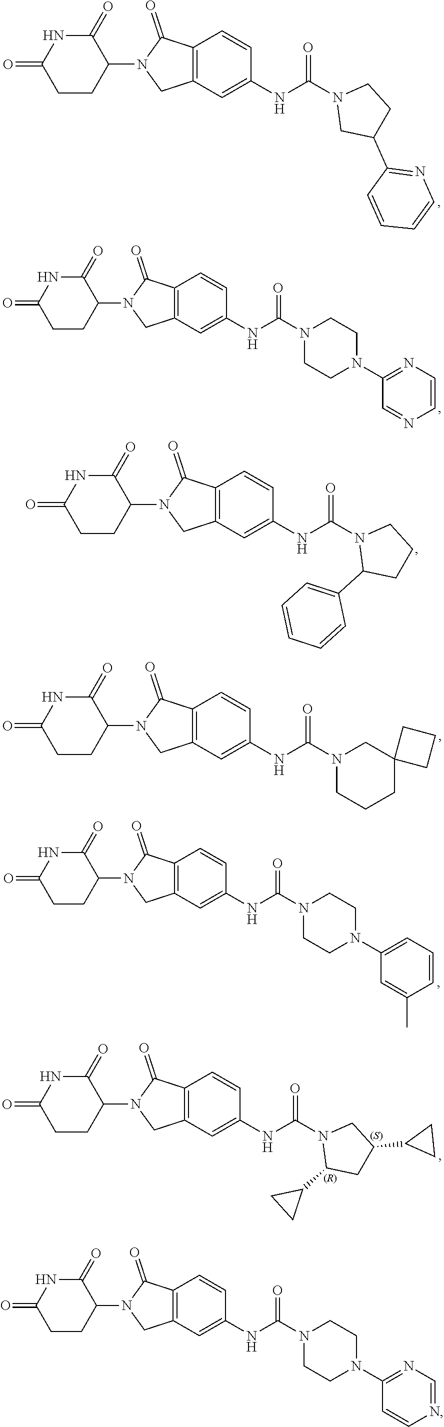 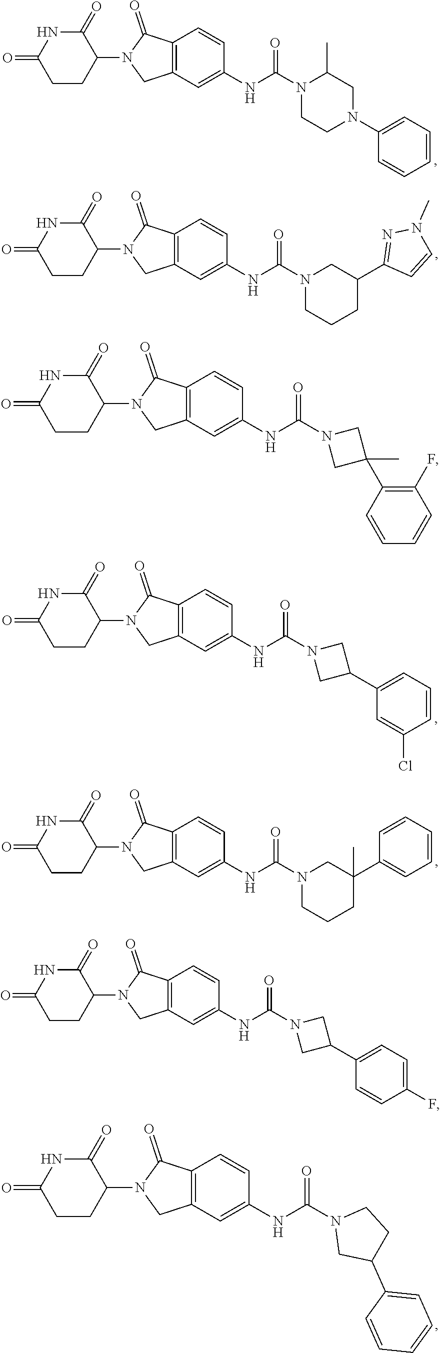 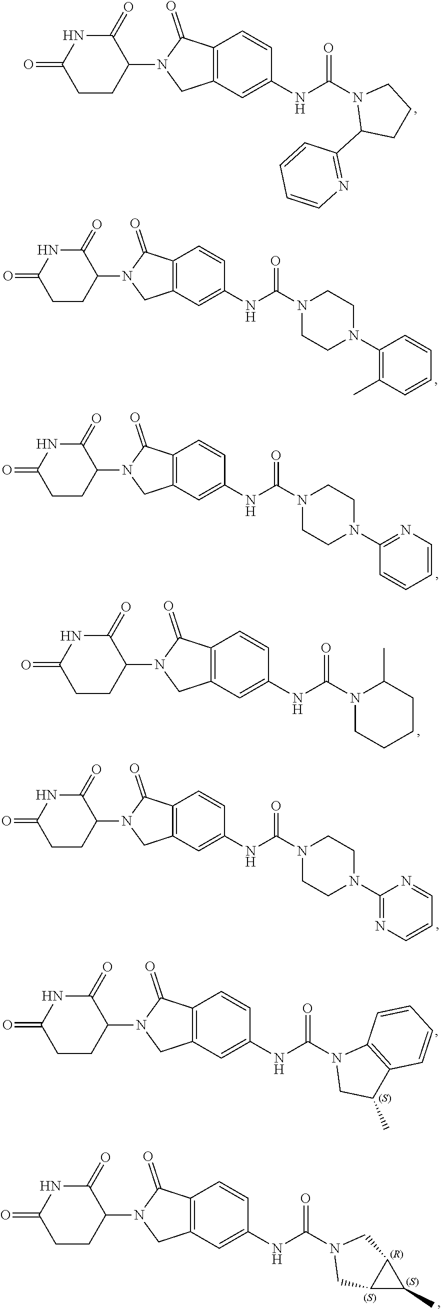 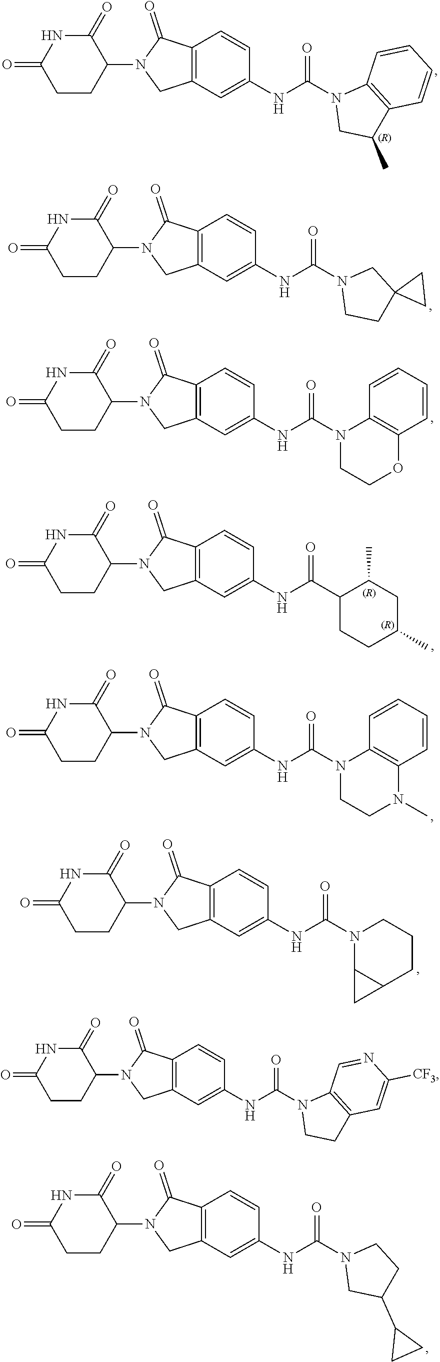 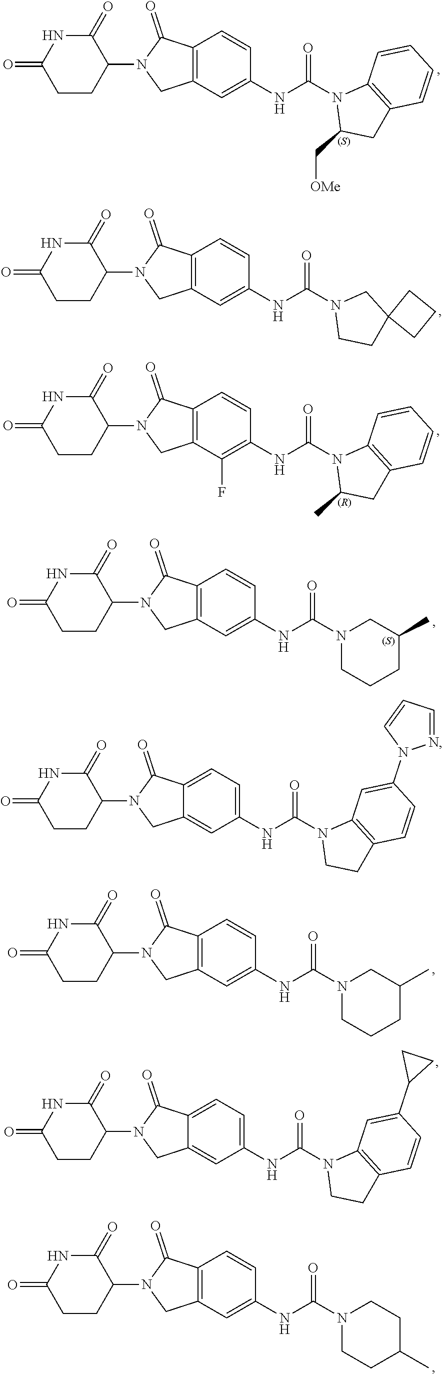 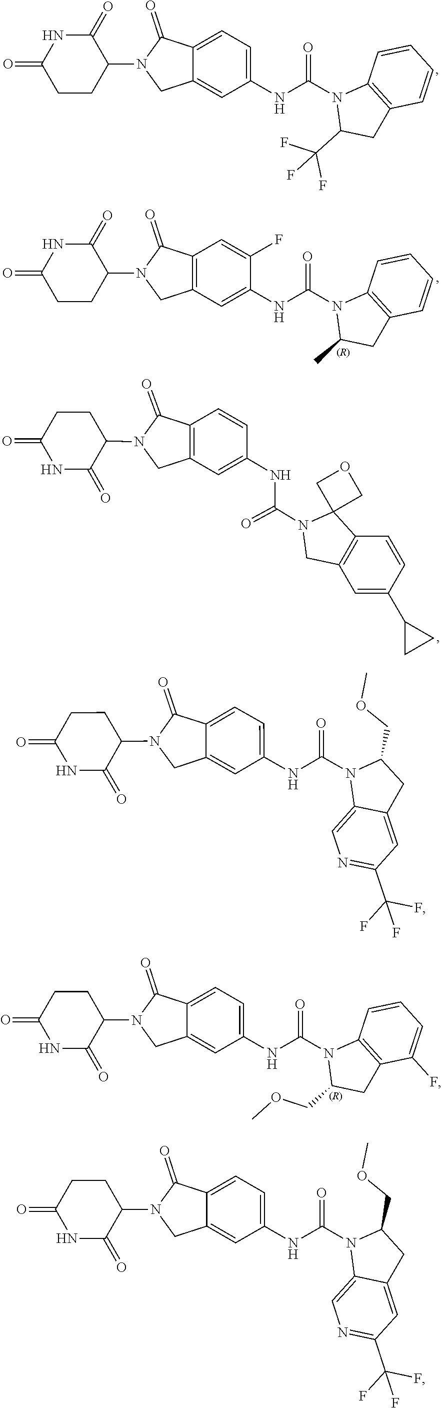 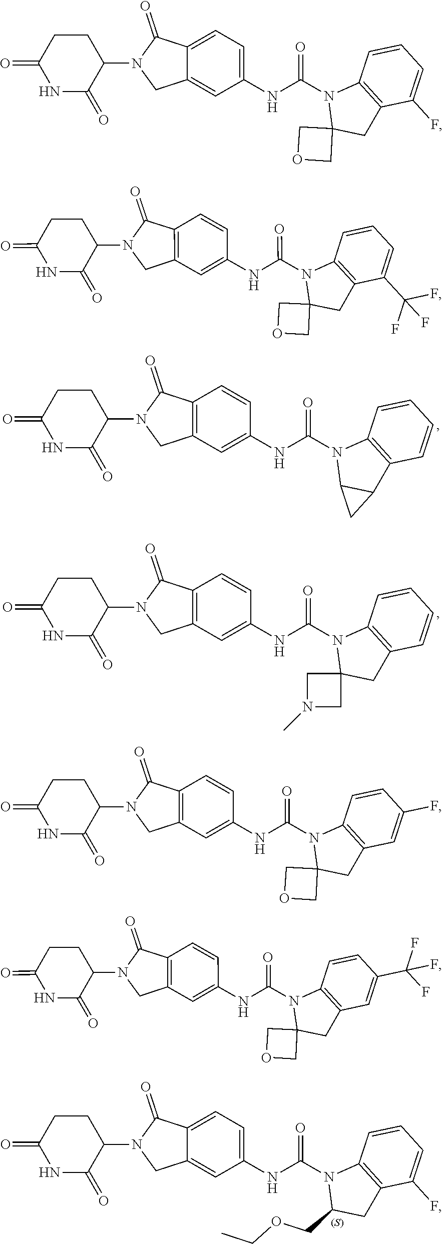 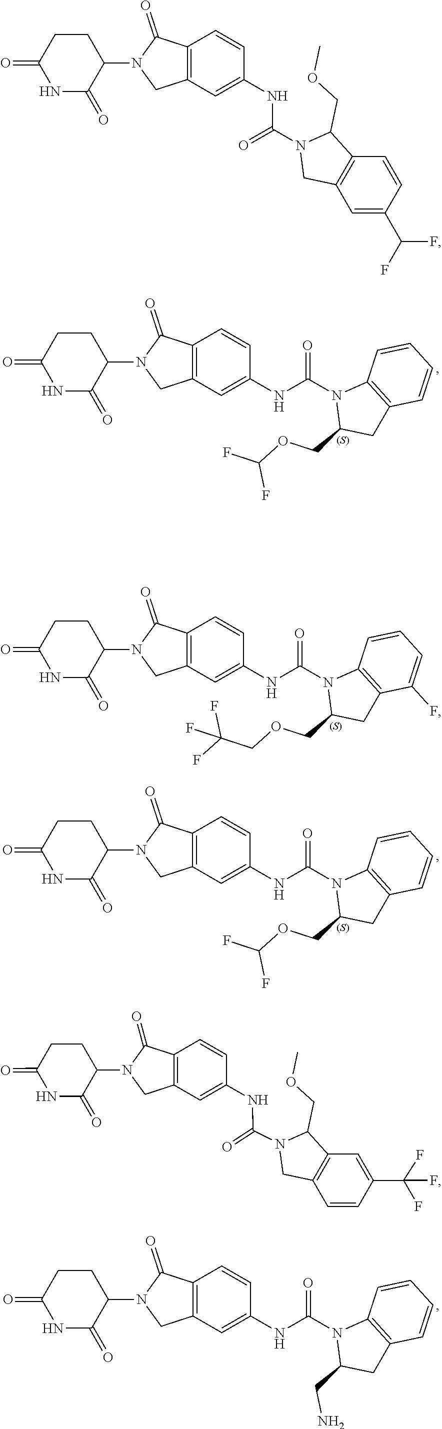 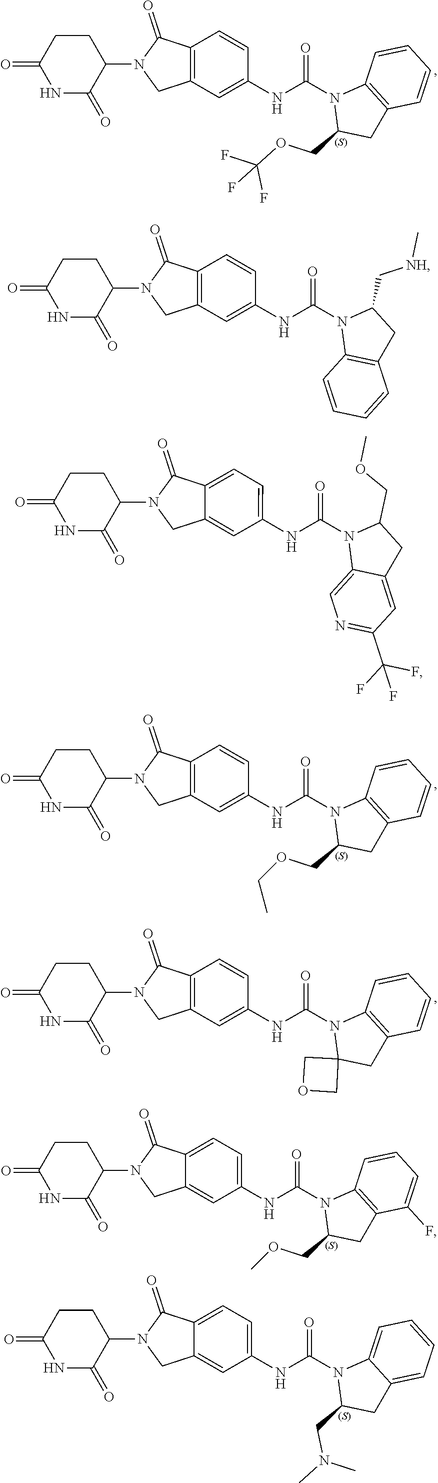 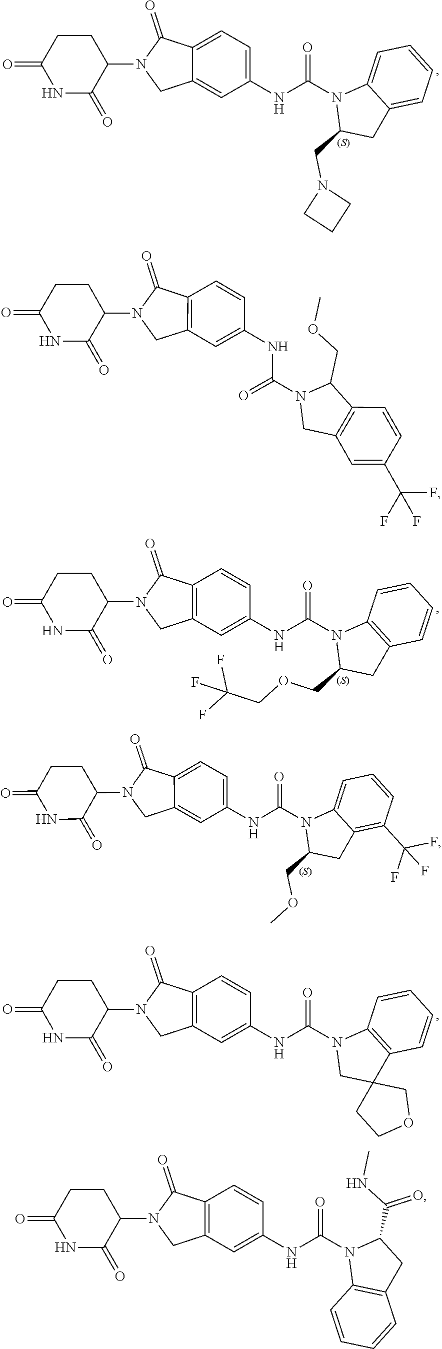 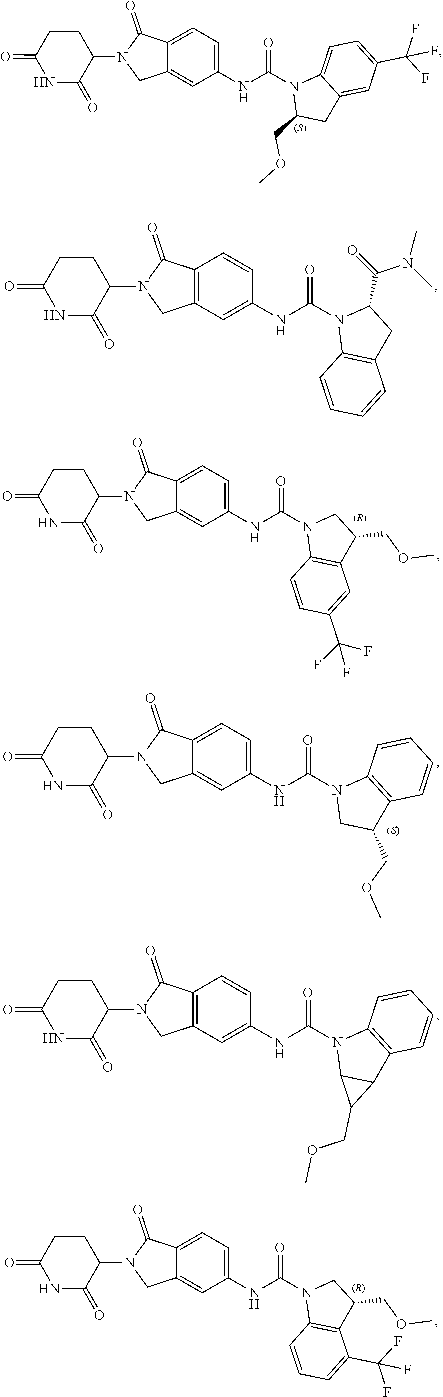 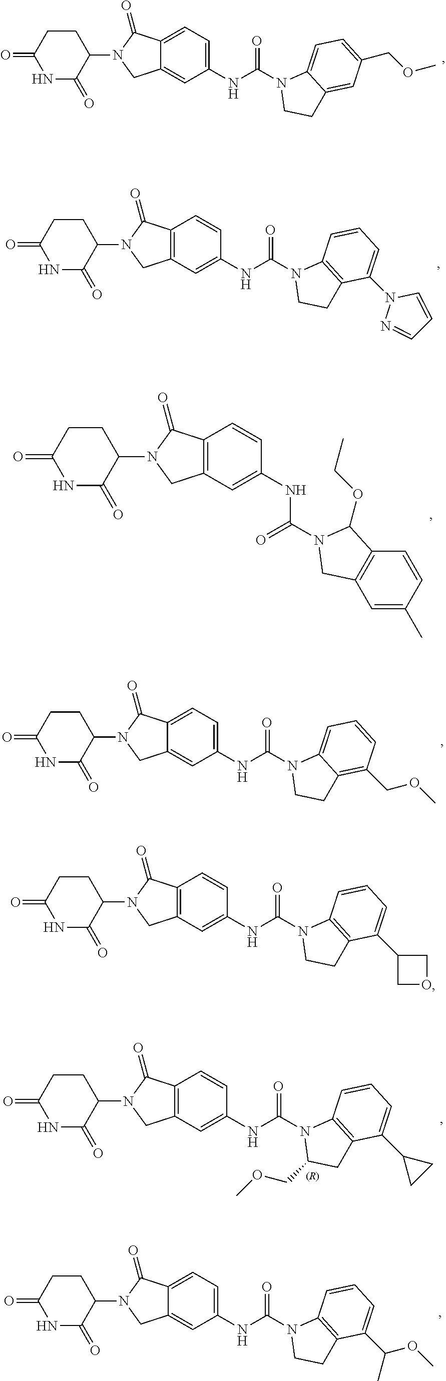 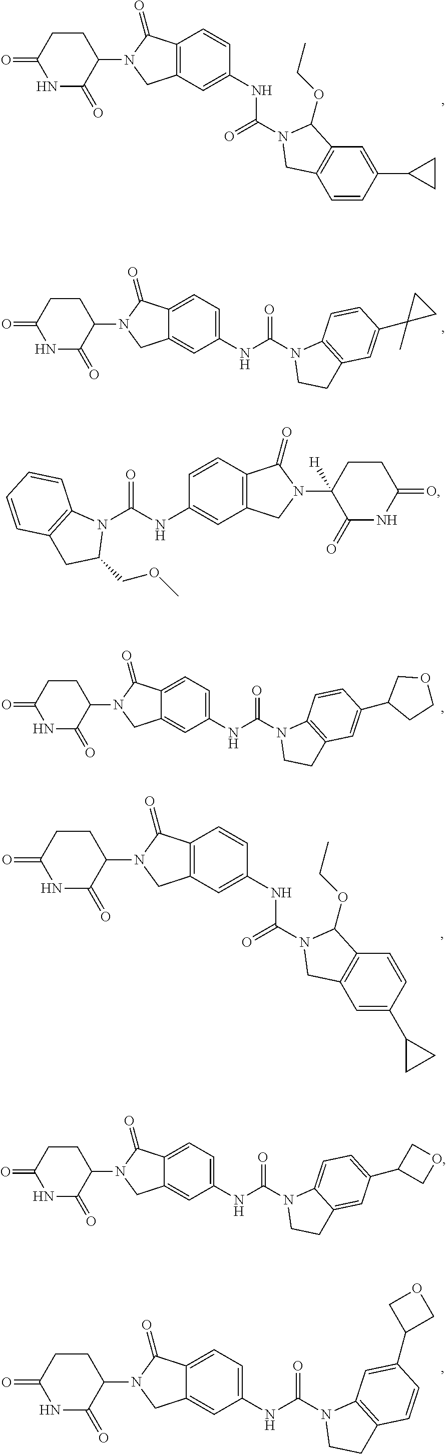 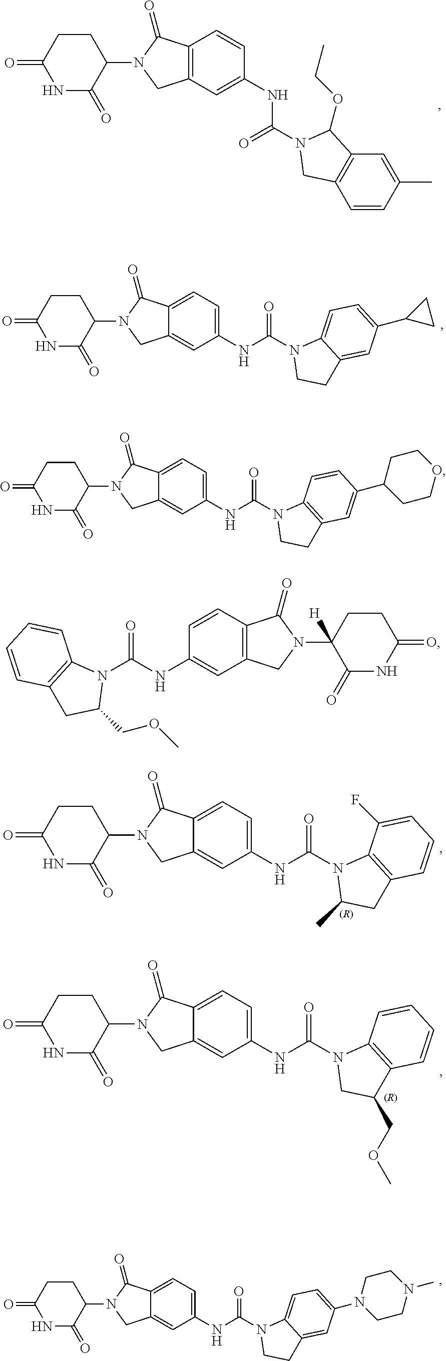 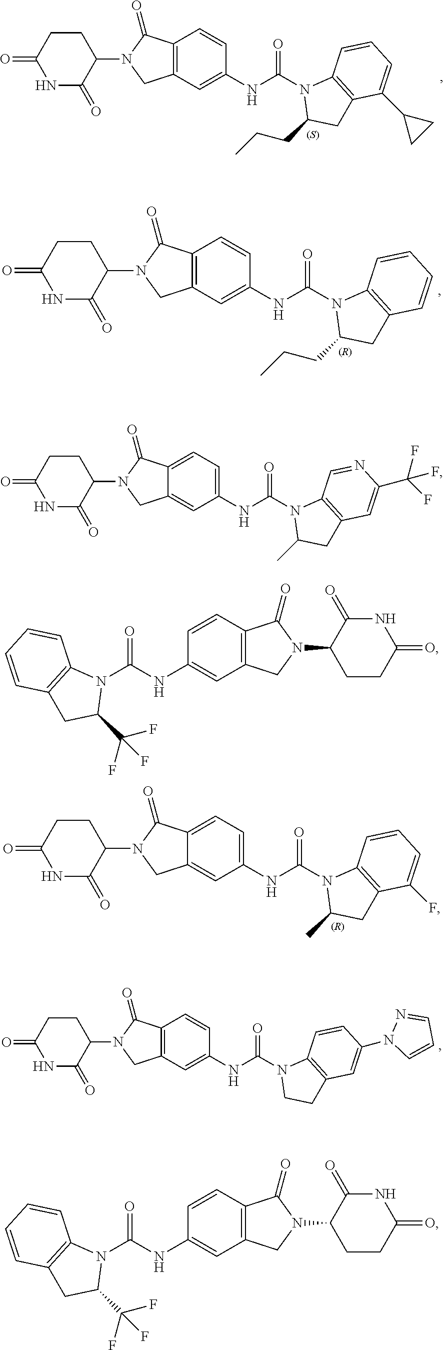 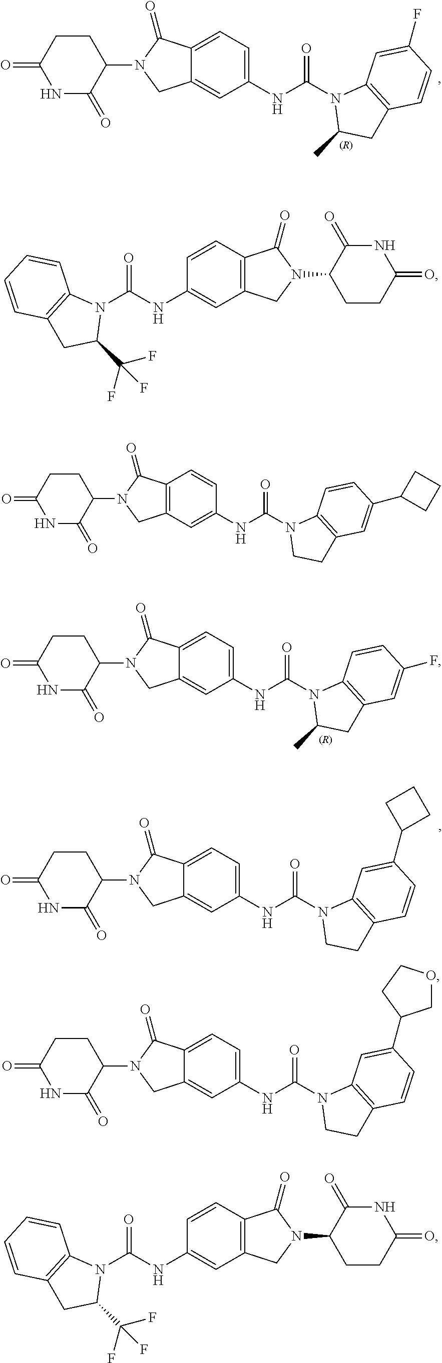 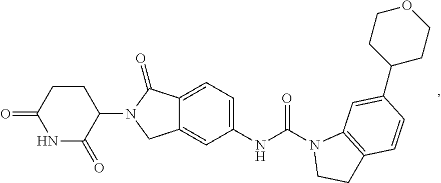 [SMILES: O=C1CCC(N2Cc3cc(NC(=O)N4CCc5ccc(C6CCOCC6)cc54)ccc3C2=O)C(=O)N1]
and pharmaceutically acceptable salts thereof.

23. A pharmaceutical composition comprising the compound of any one of claims 1-22, or pharmaceutically acceptable salt thereof, and a pharmaceutically acceptable excipient.

24. A method of degrading CK1α in a subject suffering from cancer, comprising administering to the subject an effective amount of the compound of any one of claims 1-22, or pharmaceutically acceptable salt thereof, or the pharmaceutical composition of claim 23.

25. A method of treating cancer in a subject in need thereof, comprising administering to the subject a therapeutically effective amount of the compound of any one of claims 1-22, or pharmaceutically acceptable salt thereof, or the pharmaceutical composition of claim 23.

26. A method of treating a solid tumor in a subject in need thereof, comprising administering to the subject a therapeutically effective amount of the compound of any one of claims 1-22, or pharmaceutically acceptable salt thereof, or the pharmaceutical composition of claim 23.

27. A method of treating a liquid tumor in a subject in need thereof, comprising administering to the subject a therapeutically effective amount of the compound of any one of claims 1-22, or pharmaceutically acceptable salt thereof, or the pharmaceutical composition of claim 23.

28. The method of any one of claims 24-27, further comprising administering to the subject an additional therapeutic agent.
